(19) United States
(12) Patent Application Publication
(10) Pub. No.: US 2024/0425479 A1
(43) Pub. Date: Dec. 26, 2024

(54) HALO-SUBSTITUTED AMINO AZA-HETEROARYL COMPOUNDS AS INHIBITORS OF THE HAEMATOPOIETIC PROGENITOR KINASE 1 (HPK1)

(71) Applicant: Ontario Institute for Cancer Research (OICR), Toronto, Ontario (CA)

(72) Inventors: Ahn Chau, Toronto (CA); Methvin Isaac, Brampton (CA); Babu Joseph, Oakville (CA); Radek Laufer, Oakville (CA); Gennady Poda, Toronto (CA); Michael Prakesch, Toronto (CA); David Uehling, Toronto (CA); Iain Watson, Toronto (CA); Brian Wilson, Mississauga (CA); Tao Xin, Woodbridge (CA)

(73) Assignee: Ontario Institute for Cancer Research (OICR), Toronto, ON (CA)

(21) Appl. No.: 18/288,222

(22) PCT Filed: May 2, 2022
(86) PCT No.: PCT/CA2022/050674

§ 371 Date: Oct. 25, 2023

Related U.S. Application Data
(60) Provisional application No. 63/182,249, filed on Apr. 30, 2021.

(51) Int. Cl. C07D 401/14; A61K 31/497; A61K 31/502; A61K 31/517; A61K 31/5377; A61K 31/5386; A61K 31/541; C07D 401/04; C07D 403/04; C07D 405/14; C07D 471/10; C07D 498/10

(52) U.S. Cl. CPC C07D 401/14; A61K 31/497; A61K 31/502; A61K 31/517; A61K 31/5377; A61K 31/5386; A61K 31/541; C07D 401/04; C07D 403/04; C07D 405/14; C07D 471/10; C07D 498/10

(57) ABSTRACT

The present application relates to halo-substituted aza-heteroaryl compounds of Formula (I): (I) or pharmaceutically acceptable salts, solvates and/or prodrugs thereof, to compositions comprising these compounds or pharmaceutically acceptable salts, solvates and/or prodrugs thereof, and various uses in the treatment of diseases, disorders or conditions that are treatable by inhibiting HPK1, such as cancer.

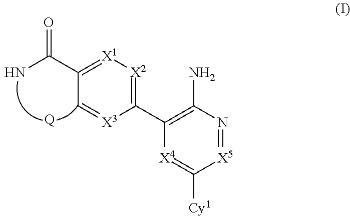

### RELATED APPLICATIONS

The present application claims the benefit of priority of co-pending U.S. provisional patent application No. 63/182,249 filed on Apr. 30, 2021, the contents of which are incorporated herein by reference in their entirety.

### FIELD

The present application relates to halo-substituted amino aza-heteroaryl compounds, to processes for their preparation, to compositions comprising them, and to their use in therapy. More particularly, it relates to halo substituted aza-heteroaryl compounds such as halo substituted amino-pyrazine and halo substituted amino-pyridazine derivatives useful in the treatment of diseases, disorders or conditions treatable by inhibiting HPK1.

### BACKGROUND

Tumors are genetically heterogeneous and have evolved mechanisms to hijack cellular growth and regulatory pathways, which makes it unlikely a single therapy will have a significant impact on patient survival. For this reason, immunotherapy has become an important paradigm in the treatment of some types of cancers. Immune effector cells such as T-cells and B-cells can suppress the proliferation of cancer cells by targeting abnormal, tumor-expressed antigens. For example, recent clinical testing of novel immunotherapy strategies (e.g. anti-PD1 and anti-PDL1) has demonstrated unprecedented and durable survival benefit even in advanced patients suffering from metastatic cancers. However, the overall excitement for this therapeutic approach is tempered by the observation that these responses to agents targeting the PD-1 axis are limited to a minority of cancer patients. Hence, in order to broaden the response rates in cancer patients, there is an urgent need to build on the tremendous promise of immunotherapy to more rapidly test rational combinations of small molecules with immuno-therapeutics. One such approach is the combination of a small molecule hematopoietic progenitor kinase 1 (HPK1) inhibitor with current anti-PD1/PDL1 immunotherapies. An HPK1 inhibitor should potentiate anti-tumor immune responses by stimulating T-cell proliferation and triggering tumor cell senescence and tumor clearance by T cells.

The hematopoietic progenitor kinase 1 (HPK1, MAP4K1), is a T-cell receptor (TCR)-proximal kinase involved in the regulation of proliferation and survival of primary T cells [Nat Immunol. 2007; 8(1):84-91.] HPK1 is exclusively expressed in hematopoietic tissues and activates the c-Jun N-terminal kinase (JNK) and the NF-kB pathways [7]. Transient knockdown of HPK1 in T cells blocks activation of NF-kB [Crit Rev Oncol Hematol. 2008; 66(1):52-64]. Most strikingly, mice that received adoptive transfer of HPKI (−/−) T cells became resistant to lung tumor growth [Immunol Res. 2012; 54(1-3):262-5]. HPK1 has an N-terminal kinase domain and a C-terminal citron homology domain. Antigen receptor cross-linking leads to activation of HPK1 in T and B cells resulting in HPK1 relocation to the plasma membrane, autophosphorylation and transphosphorylation by protein kinase D1 (PKD1). Subsequent transphosphorylation by PKD1 and auto-phosphorylation within the kinase domain result in full activation of HPK1, which then regulates different cellular responses including apoptosis, activation-induced cell death and autoimmunity. HPK1 mediates negative regulation of the immune response via phosphorylation of SLP-76 (S376). Mutation of lysine-46 to methionine (designated HPK1-M46) in the ATP-binding site of the kinase domain abolishes catalytic activation of HPK1 resulting in a kinase-dead version of the full length kinase [Genes Dev. 1996; 10 (18):2251-64]. It has been reported that HPK1 inhibition in HPKI kinase-dead knock-in mice, when treated with anti-PD-1 or anti-PDL1 antibodies demonstrate enhanced efficacy in colon cancer models relative to anti-PD-1 or anti-PDL1 treatment alone (Cell Reports 2018, 25, 80-94, and PCT Patent Application Publication Nos. WO2016/205942 and WO2016/090300). Combining or sequencing immunotherapies that target distinct immune pathways is therefore a rational strategy to increase the magnitude of the antitumor immune response over that generated with a single agent.

HPK1 plays significant roles in regulating lymphocyte receptor signaling and function. Moreover, the restricted expression of HPK1 in hematopoietic cells and the roles of HPK1 in immune cells suggest that HPK1 would be an ideal drug target for enhancing antitumor immunity. Furthermore, data from preclinical studies suggest that gene-targeted disruption of HPK1 can promote the proliferation, survival, and function of various immune cells (e.g., T cells, NK cells, and dendritic cells (DC)), and synergistically inhibit tumor growth with anti-PD-1/PDL-1 mAb. Support for this strong rationale was evident by some reports in the literature that HPK1 kinase-dead knock-in mouse bearing colorectal tumors (MC38) showed significant growth arrest treated with an anti-PD1 or anti-PDL1 antibody (PCT Patent Application Publication No. WO2016/090300). Thus, combining a small molecule that inhibits HPK1 with another immunotherapy would appear to be a rational and more effective approach toward treating cancers.

Inhibiting kinases such as HPK1 therefore represents promising targets for immunooncology due to their role in limiting T-cell activation. At the same time, in the pursuit of these targets it is desirable that there be selectivity against other kinases that are involved in a robust T cell activation. An example of such a kinase includes, but is not limited to Lck (Sawasdikosol, et. al. Structure 27, 2019, 1-3).

### SUMMARY

Current cancer immunotherapy strategies seek to reverse immune tolerance either by modulating T cell co-receptor signals or boosting the recognition of tumor-associated antigens by using native biomolecules or mAbs. Selective HPK1 inhibitors, combined with other immuno-modulating agents should amplify the anti-tumor activity of immune cells. The present application discloses novel compounds that have such activity.

Accordingly, the present application includes a compound of Formula (I) or a pharmaceutically acceptable salt, solvate and/or prodrug thereof,

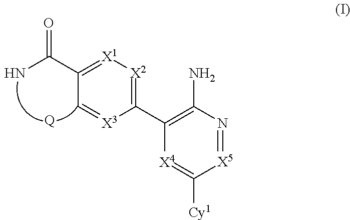

wherein:
X¹ is selected from N and CR¹;
X² and X³ are independently selected from N and CR²;
one of X⁴ and X⁵ is N and the other is CR³;
Q is C₁₋₄alkylene optionally interrupted by a heteroatom selected from O, S, S(O), SO₂, and NR⁴ and/or optionally substituted with one or more of R⁵ and/or optionally disubstituted on one carbon with R^{5a} and R^{5b}; provided that when Q comprises the heteromoiety the heteromoiety is not separated from the ring amide NH by methylene; or
Q is C₂₋₄alkenylene optionally substituted with one or more of R^{5c}; or
Q is optionally selected from C═N and N═C, and is optionally substituted with R^{5c};
R¹ is selected from H, halo, OR⁶, NR⁷R⁸, C₁₋₆alkyleneNR⁷R⁸ and C₁₋₆alkyl;
R² is selected from H, halo and C₁₋₆alkyl;
R³ is halo;
R⁴ is selected from H and C₁₋₆alkyl;
each R⁵ is independently selected from ═O, halo, C₁₋₆alkyl, C₃₋₆cycloalkyl, C₃₋₆heterocycloalkyl, C₁₋₆alkyleneC₃₋₆cycloalkyl, C₁₋₆alkyleneC₃₋₆heterocycloalkyl, OH, OC₁₋₆alkyl, NR⁹R¹⁰ and C₁₋₆alkyleneNR⁹R¹⁰;
R^{5a} and R^{5b} are joined to form, together with the carbon atom therebetween, a 3- to 6-membered, saturated or unsaturated ring optionally containing one heteromoiety selected from N, NH, NC₁₋₆alkyl, O, S, S(O), and SO₂ and optionally substituted with one or more of halo and C₁₋₆alkyl;
each R^{5c} is independently selected from halo, C₁₋₆alkyl, C₃₋₆cycloalkyl, C₃₋₆heterocycloalkyl, C₁₋₆alkyleneC₃₋₆cycloalkyl, C₁₋₆alkyleneC₃₋₆heterocycloalkyl, OH, OC₁₋₆alkyl, NR⁹R¹⁰ and C₁₋₆alkyleneNR⁹R¹⁰;
R⁶ is selected from H and C₁₋₆alkyl;
R⁷, R⁸, R⁹ and R¹⁰ are each independently selected from H and C₁₋₆alkyl; or
R⁷ and R⁸ or R⁹ and R¹⁰ are joined to form, together with the nitrogen atom therebetween, a 3- to 7-membered saturated or unsaturated ring, optionally containing one additional heteromoiety selected from N, NH, NC₁₋₆alkyl, O, S, S(O), and SO₂ and optionally substituted with one or more of halo and C₁₋₆alkyl;
Cy¹ is C₆₋₂₀aryl or C₅₋₂₀heteroaryl, and Cy¹ is unsubstituted or is substituted with one or more of R¹¹, or
Cy¹ is substituted with Z-Cy², or
Cy¹ is substituted Z-Cy² and with one or more of R¹¹;
each R¹¹ is independently selected from halo, ═O, CN, NO₂, C₁₋₆alkyl, C₂₋₆alkenyl, C₂₋₆alkynyl, OR¹², C(O)R¹², CO₂R¹², P(O)R¹²R¹³, P(O)(OR¹²)(OR¹³), SR¹², S(O)R¹², SO₂R¹², S(O)(═NR¹³)R¹², SO₂NR¹²R¹³, SiR¹⁴R^{14a}R^{14b}, C₁₋₆alkyleneOR¹², OC₁₋₆alkyleneOR¹², C₁₋₆alkyleneNR^{12a}R^{13a}, OC₁₋₆alkyleneNR¹²R¹³, NR^{13a}C₁₋₆alkyleneNR¹²R¹³, NR^{13a}C₁₋₆alkyleneOR¹², NR^{12a}R^{13a}, NR^{13a}COR¹², NR^{13a}CO₂R¹², NR^{13a}SO₂R¹², C₃₋₇cycloalkyl, C₃₋₇heterocycloalkyl, C₁₋₆alkyleneC₃₋₇cycloalkyl and C₁₋₆alkyleneC₃₋₇heterocycloalkyl, the latter four groups being optionally substituted with one or more of R¹⁵;
R¹² is selected from H, C₁₋₆alkyl, C₂₋₆alkenyl, C₂₋₆alkynyl, C₁₋₆alkyleneC₃₋₁₀cycloalkyl, C₁₋₆alkyleneC₃₋₁₀heterocycloalkyl, C₁₋₆alkyleneOR¹⁶, and C₁₋₆alkyleneNR^{16a}R^{16b}, and alkyl, alkenyl, alkynyl, alkylene, heterocycloalkyl and cycloalkyl groups of R¹² are optionally substituted with one or more of R¹⁷;
R¹³ is selected from H and C₁₋₆alkyl; or
R¹² and R¹³ are joined to form, together with the atom(s) therebetween, a 4- to 6-membered saturated or unsaturated ring, optionally containing one additional heteromoiety selected from N, NR¹⁸, O, S, SO, and SO₂, and optionally substituted with one or more of R¹⁷;
R^{12a} is selected from H, C₁₋₆alkyl, C₂₋₆alkenyl, C₂₋₆alkynyl, C₁₋₆alkyleneC₃₋₁₀cycloalkyl, C₁₋₆alkyleneC₃₋₁₀heterocycloalkyl, C₁₋₆alkyleneOR¹⁶, and C₁₋₆alkyleneNR^{16a}R^{16b}, and all alkyl, alkenyl, alkynyl, alkylene, heterocycloalkyl and cycloalkyl groups of R^{12a} are optionally substituted with one or more of R¹⁷;
R^{13a} is selected from H and C₁₋₆alkyl;
R¹⁴, R^{14a}, and R^{14b} are independently selected from OR¹⁹, C₁₋₆alkyl, C₃₋₁₁cycloalkyl, C₃₋₁₀heterocycloalkyl, C₁₋₆alkyleneC₃₋₁₀cycloalkyl, and C₁₋₆alkyleneC₃₋₁₀heterocycloalkyl;
each R¹⁵ is independently selected from halo, OH, C₁₋₆alkyl, OC₁₋₆alkyl, CN and NR^{15a}R^{15b};
R^{15a} and R^{15b} are each independently selected from H and C₁₋₆alkyl;
R¹⁶, R^{16a} and R^{16b} are independently selected from H and C₁₋₆alkyl;
each R¹⁷ is independently selected from halo, C₁₋₆alkyl, CN and NR^{17a}R^{17b};
R^{17a} and R^{17b} are each independently selected from H and C₁₋₆alkyl;
R¹⁸ is selected from H and C₁₋₆alkyl;
R¹⁹ is selected from H, C₁₋₆alkyl, C₃₋₁₁cycloalkyl, C₃₋₁₀heterocycloalkyl, C₁₋₆alkyleneC₃₋₁₀cycloalkyl, C₁₋₆alkyleneC₃₋₁₀heterocycloalkyl, C₁₋₆alkyleneOR²⁰ and C₁₋₆alkyleneNR²⁰R²¹;
R²⁰ and R²¹ are independently selected from H and C₁₋₆alkyl;
Z is absent, or is selected from C₁₋₆alkylene, O, C(O), CO₂, S, S(O), SO₂, S(O)(═NR^{13b}) and NR^{13b}, C₁₋₆alkyleneO, C₁₋₆alkyleneC(O), C₁₋₆alkyleneCO₂, C₁₋₆alkyleneS, C₁₋₆alkyleneS(O), C₁₋₆alkyleneSO₂, C₁₋₆alkyleneS(O)(═NR^{13b}), C₁₋₆alkyleneNR^{13b}, OC₁₋₆alkylene, C(O)C₁₋₆alkylene, CO₂C₁₋₆alkylene, SC₁₋₆alkylene, S(O)C₁₋₆alkylene, SO₂C₁₋₆alkylene, S(O)(═NR^{13b})C₁₋₆alkylene and NR^{13b}C₁₋₆alkylene;
R^{13b} is selected from H and C₁₋₆alkyl;
Cy² is selected from C₃₋₁₄cycloalkyl and C₃₋₁₄heterocycloalkyl, and Cy² is unsubstituted or substituted with one or more of R²²;
each R²² is independently selected from halo, ═O, CN, OH, C₁₋₆alkyl, C₂₋₆alkenyl, C₂₋₆alkynyl, C₃₋₁₀cycloalkyl, C₃₋₁₀heterocycloalkyl, C₆₋₁₁aryl, C₅₋₁₄heteroaryl, C₁₋₆alkyleneC₃₋₁₀cycloalkyl, C₁₋₆alkyleneC₃₋₁₀heterocycloalkyl, C₁₋₆alkyleneC₆₋₁₁aryl, C₁₋₆alkyleneC₅₋₁₄heteroaryl OC₁₋₆alkyl, OC₂₋₆alkenyl, OC₂₋₆alkynyl, C₁₋₆alkyleneOR²³, OC₁₋₆alkyleneOR²³, C₁₋₆alkyleneNR²⁴R²⁵, OC₁₋₆alkyleneNR²⁴R²⁵, SC₁₋₆alkyl, SC₂₋₆alkenyl, SC₂₋₆alkynyl, C(O)C₁₋₆alkyl, C(O)C₂₋₆alkenyl, C(O)C₂₋₆alkynyl, C(O)C₃₋₁₀cycloalkyl, C(O)C₃₋₁₀heterocycloalkyl, C(O)C₆₋₁₁aryl, C(O)C₅₋₁₄heteroaryl, C(O)C₁₋₆alkyleneC₃₋₁₀cycloalkyl, C(O)C₁₋₆alkyleneC₃₋₁₀heterocycloalkyl, C(O)C₁₋₆alkyleneC₆₋₁₁aryl, C(O)C₁₋₆alkyleneC₅₋₁₄heteroaryl, C(O)C₁₋₆alkylenylOR²³, C(O)C₁₋₆alkyleneNR²⁴R²⁵, C(O)C₁₋₆alkyleneOC₁₋₆alkyleneNR²⁴R²⁵, C(O)NR²⁴R²⁵, CO₂C₁₋₆alkyl, CO₂C₂₋₆alkenyl, CO₂C₂₋₆alkynyl, CO₂C₁₋₆alkyleneOR²³, CO₂C₁₋₆alkyleneOC₁₋₆alkyleneNR²⁴R²⁵, NR²⁴R²⁵, NR²⁶C₃₋₁₀cycloalkyl, NR²⁶C₃₋₁₀heterocycloalkyl, NR²⁶C₆₋₁₁aryl, NR²⁶C₅₋₁₄heteroaryl, NR²⁶C₁₋₆alkyleneOR²³, NR²⁶C₁₋₆alkylene NR²⁴R²⁵, NR²⁶C₁₋₆alkyleneC₃₋₁₀cycloalkyl, NR²⁶C₁₋₆alkyleneC₃₋₁₀heterocycloalkyl, NR²⁶C₁₋₆alkyleneC₆₋₁₁ aryl, NR²⁶C₁₋₆alkyleneC₅₋₁₄heteroaryl, NR²⁶SO₂C₁₋₆alkyl, SO₂C₁₋₆alkyl, SO₂C₂₋₆alkenyl, SO₂C₂₋₆alkynyl, and SO₂NR²⁴R²⁵, and alkyl, alkenyl, alkynyl, alkylene, aryl, heteroaryl, heterocycloalkyl and cycloalkyl groups of R²² are optionally substituted with one or more of R²⁷;
R²³ is selected from H, C₁₋₆alkyl, C₁₋₆alkyleneOC₁₋₆alkyl, C₃₋₁₁cycloalkyl, C₃₋₁₀heterocycloalkyl, C₆₋₁₁aryl, C₅₋₁₄heteroaryl, C₂₋₆alkenyl, C₂₋₆alkynyl, C₁₋₆alkyleneC₃₋₁₀cycloalkyl, C₁₋₆alkyleneC₃₋₁₀heterocycloalkyl, C₁₋₆alkyleneC₆₋₁₁aryl, and C₁₋₆alkyleneC₅₋₁₄heteroaryl;
R²⁴ is selected from H and C₁₋₆alkyl;
R²⁵ is selected from H, C₁₋₆alkyl, C₁₋₆alkyleneOC₁₋₆alkyl, C₃₋₁₀cycloalkyl, C₃₋₁₀heterocycloalkyl, C₆₋₁₁aryl, C₅₋₁₄heteroaryl, C₁₋₆alkyleneC₃₋₁₀cycloalkyl, C₁₋₆alkyleneC₃₋₁₀heterocycloalkyl, C₁₋₆alkyleneC₆₋₁₁aryl, and C₁₋₆alkyleneC₅₋₁₄heteroaryl, or
R²⁴ and R²⁵ are joined to form, together with the nitrogen atom therebetween, a 4- to 6-membered saturated or unsaturated ring, optionally containing one additional heteromoiety selected from N, NR²⁸, O, S, S(O), and SO₂, and optionally substituted with one or more of halo and C₁₋₆alkyl;
R²⁶ is selected from H and C₁₋₆alkyl;
each R²⁷ is independently selected from halo, C₁₋₆alkyl, CN and NR^{27a}R^{27b}; and
R^{27a}, R^{27b}, and R²⁸ are each independently selected from H and C₁₋₆alkyl;
wherein all available hydrogen atoms are optionally substituted with a fluorine atom.

The present application also includes a composition comprising one or more compounds of the application and a carrier. In an embodiment, the composition is a pharmaceutical composition comprising one or more compounds of the application and a pharmaceutically acceptable carrier.

In an embodiment, the compounds of the application are used as medicaments. Accordingly, the application also includes a compound of the application for use as a medicament.

The compounds of the application have been shown to inhibit HPK1. Therefore the compounds of the application are useful for treating diseases, disorders or conditions that are treatable by inhibiting HPK1 activity. Accordingly, the present application also includes a method of treating a disease, disorder or condition that is treatable by inhibiting HPK1, comprising administering a therapeutically effective amount of one or more compounds of the application to a subject in need thereof.

The present application also includes a use of one or more compounds of the application for treatment of a disease, disorder or condition that is treatable by inhibiting HPK1, as well as a use of one or more compounds of the application for the preparation of a medicament for treatment of a disease, disorder or condition that is treatable by inhibiting HPK1. The application further includes one or more compounds of the application for use in treating a disease, disorder or condition that is treatable by inhibiting HPK1.

In a further embodiment, the disease, disorder or condition that is treatable by inhibiting HPK1 is cancer and the one or more compounds of the application are administered in combination with one or more additional cancer treatments. In another embodiment, the additional cancer treatment is selected from radiotherapy, chemotherapy, targeted therapies such as antibody therapies and small molecule therapies such as tyrosine-kinase inhibitors, immunotherapy, hormonal therapy and anti-angiogenic therapies. In another embodiment, the additional cancer treatment is selected from an antibody that binds to PD-1 or PDL-1.

The application additionally provides a process for the preparation of compounds of the application. General and specific processes are discussed in more detail and set forth in the Examples below.

Other features and advantages of the present application will become apparent from the following detailed description. It should be understood, however, that the detailed description and the specific examples, while indicating embodiments of the application, are given by way of illustration only and the scope of the claims should not be limited by these embodiments, but should be given the broadest interpretation consistent with the description as a whole.

### DETAILED DESCRIPTION

### I. Definitions

Unless otherwise indicated, the definitions and embodiments described in this and other sections are intended to be applicable to all embodiments and aspects of the present application herein described for which they are suitable as would be understood by a person skilled in the art.

All features disclosed in the specification, including the claims, abstract, and drawings, and all the steps in any method or process disclosed, may be combined in any combination, except combinations where at least some of such features and/or steps are mutually exclusive. Each feature disclosed in the specification, including the claims, abstract, and drawings, can be replaced by alternative features serving the same, equivalent, or similar purpose, unless expressly stated otherwise.

As used in this application and claim(s), the words “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”), are inclusive or open-ended and do not exclude additional, unrecited elements or process steps.

The term “consisting” and its derivatives as used herein are intended to be closed terms that specify the presence of the stated features, elements, components, groups, integers, and/or steps, and also exclude the presence of other unstated features, elements, components, groups, integers and/or steps.

The term “consisting essentially of”, as used herein, is intended to specify the presence of the stated features, elements, components, groups, integers, and/or steps as well as those that do not materially affect the basic and novel characteristic(s) of these features, elements, components, groups, integers, and/or steps.

The terms “about”, “substantially” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies or unless the context suggests otherwise to a person skilled in the art.

As used in the present application, the singular forms “a”, “an” and “the” include plural references unless the content clearly dictates otherwise. For example, an embodiment including “a compound” should be understood to present certain aspects with one compound, or two or more additional compounds.

In embodiments comprising an “additional” or “second” component or effect, such as an additional or second compound, the second compound as used herein is different from the other compounds or first compound. A “third” compound is different from the other, first, and second compounds, and further enumerated or “additional” compounds are similarly different.

The term “and/or” as used herein means that the listed items are present, or used, individually or in combination. In effect, this term means that “at least one of” or “one or more” of the listed items is used or present. The term “and/or” with respect to enantiomers, prodrugs, salts and/or solvates thereof means that the compounds of the application exist as individual enantiomers, prodrugs, salts and hydrates, as well as a combination of, for example, a salt of a solvate of a compound of the application.

The term “compound of the application” or “compound of the present application” and the like as used herein refers to a compound of Formula (I) or Formula I-A I-B, I-C, I-D, I-E, I-F, I-G, I-H, I-J, I-K, I-L, I-M, I-N, I-O, I-P, I-Q, I-R, I-S, I-T and I-U, or salts, solvates and/or prodrugs thereof.

The term “composition of the application” or “composition of the present application” and the like as used herein refers to a composition comprising one or more compounds of the application.

The term “suitable” as used herein means that the selection of the particular compound or conditions would depend on the specific synthetic manipulation to be performed, the identity of the molecule(s) to be transformed and/or the specific use for the compound, but the selection would be well within the skill of a person trained in the art.

The present description refers to a number of chemical terms and abbreviations used by those skilled in the art. Nevertheless, definitions of selected terms are provided for clarity and consistency.

The term “protecting group” or “PG” and the like as used herein refers to a chemical moiety which protects or masks a reactive portion of a molecule to prevent side reactions in those reactive portions of the molecule, while manipulating or reacting a different portion of the molecule. After the manipulation or reaction is complete, the protecting group is removed under conditions that do not degrade or decompose the remaining portions of the molecule. The selection of a suitable protecting group can be made by a person skilled in the art. Many conventional protecting groups are known in the art, for example as described in “Protective Groups in Organic Chemistry” McOmie, J. F. W. Ed., Plenum Press, 1973, in Greene, T. W. and Wuts, P. G. M., “Protective Groups in Organic Synthesis”, John Wiley & Sons, 3^{rd} Edition, 1999 and in Kocienski, P. Protecting Groups, 3rd Edition, 2003, Georg Thieme Verlag (The Americas).

The term “inert organic solvent” as used herein refers to a solvent that is generally considered as non-reactive with the functional groups that are present in the compounds to be combined together in any given reaction so that it does not interfere with or inhibit the desired synthetic transformation. Organic solvents are typically non-polar and dissolve compounds that are non soluble in aqueous solutions.

The term “alkyl” as used herein, whether it is used alone or as part of another group, means straight or branched chain, saturated alkyl groups. The number of carbon atoms that are possible in the referenced alkyl group are indicated by the prefix “Cₙ₁₋ₙ₂”. For example, the term C₁₋₁₀alkyl means an alkyl group having 1, 2, 3, 4, 5, 6, 7, 8, 9 or 10 carbon atoms. All alkyl groups are optionally fluoro-substituted.

The term “alkylene”, whether it is used alone or as part of another group, means straight or branched chain, saturated alkylene group, that is, a saturated carbon chain that contains substituents on two of its ends. The number of carbon atoms that are possible in the referenced alkylene group are indicated by the prefix “Cₙ₁₋ₙ₂”. For example, the term C₂₋₆alkylene means an alkylene group having 2, 3, 4, 5 or 6 carbon atoms. All alkylene groups are optionally fluoro-substituted.

The term “alkenyl” as used herein, whether it is used alone or as part of another group, means straight or branched chain, unsaturated alkyl groups containing at least one double bond. The number of carbon atoms that are possible in the referenced alkylene group are indicated by the prefix “Cₙ₁₋ₙ₂”. For example, the term C₂₋₆alkenyl means an alkenyl group having 2, 3, 4, 5 or 6 carbon atoms and at least one double bond. All alkenyl groups are optionally fluoro-substituted.

The term “alkynyl” as used herein, whether it is used alone or as part of another group, means straight or branched chain, unsaturated alkynyl groups containing at least one triple bond. The number of carbon atoms that are possible in the referenced alkyl group are indicated by the prefix “Cₙ₁₋ₙ₂”. For example, the term C₂₋₆alkynyl means an alkynyl group having 2, 3, 4, 5 or 6 carbon atoms. All alkynyl groups are optionally fluoro-substituted.

The term “cycloalkyl,” as used herein, whether it is used alone or as part of another group, means a saturated carbocyclic group containing from 3 to 20 carbon atoms and one or more rings. The number of carbon atoms that are possible in the referenced cycloalkyl group are indicated by the numerical prefix “Cₙ₁₋ₙ₂”. For example, the term C₃₋₁₀cycloalkyl means a cycloalkyl group having 3, 4, 5, 6, 7, 8, 9 or 10 carbon atoms. All cycloalkyl groups are optionally fluoro-substituted.

The term “aryl” as used herein, whether it is used alone or as part of another group, refers to carbocyclic groups containing at least one aromatic ring and contains either 6 to 20 carbon atoms. All aryl groups are optionally fluoro-substituted.

The term “heterocycloalkyl” as used herein, whether it is used alone or as part of another group, refers to cyclic groups containing at least one non-aromatic ring containing from 3 to 20 atoms in which one or more of the atoms are a heteroatom selected from O, S and N and the remaining atoms are C. Heterocycloalkyl groups are either saturated or unsaturated (i.e. contain one or more double bonds). When a heterocycloalkyl group contains the prefix Cₙ₁₋ₙ₂ this prefix indicates the number of carbon atoms in the corresponding carbocyclic group, in which one or more, suitably 1 to 5, of the ring atoms is replaced with a heteroatom as selected from O, S and N and the remaining atoms are C. Heterocycloalkyl groups are optionally benzofused. The heteroatom in heterocycloalkyl groups is optionally substituted or oxidized where valency allows. All heterocycloalkyl groups are optionally fluoro-substituted.

The term “heteroaryl” as used herein, whether it is used alone or as part of another group, refers to cyclic groups containing at least one heteroaromatic ring containing 5-20 atoms in which one or more of the atoms are a heteroatom selected from O, S and N and the remaining atoms are C. When a heteroaryl group contains the prefix Cₙ₁₋ₙ₂ this prefix indicates the number of carbon atoms in the corresponding carbocyclic group, in which one or more, suitably 1 to 5, of the ring atoms is replaced with a heteroatom as defined above. Heteroaryl groups are optionally benzofused. The heteroatom in heteroaryl groups is optionally substituted or oxidized where valency allows. All heteroaryl groups are optionally fluoro-substituted.

The term “aza-heteroaryl” as used herein, whether it is used alone or as part of another group, refers to a heteroaryl group having two or more N atoms as the only heteroatom in the group. All aza-heteroaryl groups are optionally fluoro-substituted.

All cyclic groups, including aryl, heteroaryl, heterocycloalkyl and cycloalkyl groups, contain one or more than one ring (i.e. are polycyclic). When a cyclic group contains more than one ring, the rings may be fused, bridged, spirofused or linked by a bond.

The term “benzofused” as used herein refers to a polycyclic group in which a benzene ring is fused with another ring.

A first ring being “fused” with a second ring means the first ring and the second ring share two adjacent atoms there between.

A first ring being “bridged” with a second ring means the first ring and the second ring share two non-adjacent atoms there between.

A first ring being “spirofused” with a second ring means the first ring and the second ring share one atom there between.

The term “fluoro-substituted” refers to the substitution of one or more, including all, available hydrogens in a referenced group with fluoro.

The terms “halo” or “halogen” as used herein, whether it is used alone or as part of another group, refers to a halogen atom and includes fluoro, chloro, bromo and iodo.

The term “available”, as in “available hydrogen atoms” or “available atoms” refers to atoms that would be known to a person skilled in the art to be capable of replacement by a substituent.

When a group is said to be substituted with multiple substituents, said substituents are independently selected therefore can be the same or different.

The term “cross-coupling” as used herein refers to chemical reactions in which two different starting materials, each of which is usually endowed with an activating group, are reacted together with the aid of a metal catalyst. The result is the loss of the two activating groups and the formation of a new covalent bond between the remaining fragments.

The term “cell” as used herein refers to a single cell or a plurality of cells and includes a cell either in a cell culture or in a subject.

The term “subject” as used herein includes all members of the animal kingdom including mammals, and suitably refers to humans. Thus the methods and uses of the present application are applicable to both human therapy and veterinary applications.

The term “pharmaceutically acceptable” means compatible with the treatment of subjects, for example humans.

The term “pharmaceutically acceptable carrier” means a non-toxic solvent, dispersant, excipient, adjuvant or other material which is mixed with the active ingredient in order to permit the formation of a pharmaceutical composition, i.e., a dosage form capable of administration to a subject.

The term “pharmaceutically acceptable salt” means either an acid addition salt or a base addition salt which is suitable for, or compatible with the treatment of subjects.

The term “solvate” as used herein means a compound, or a salt and/or prodrug of a compound, wherein molecules of a suitable solvent are incorporated in the crystal lattice. A suitable solvent is physiologically tolerable at the dosage administered.

The term “prodrug” as used herein means a compound, or salt and/or solvate of a compound, that, after administration, is converted into an active drug.

The term “treating” or “treatment” as used herein and as is well understood in the art, means an approach for obtaining beneficial or desired results, including clinical results. Beneficial or desired clinical results can include, but are not limited to alleviation or amelioration of one or more symptoms or conditions, diminishment of extent of disease, stabilized (i.e. not worsening) state of disease, preventing spread of disease, delay or slowing of disease progression, amelioration or palliation of the disease state, diminishment of the reoccurrence of disease, and remission (whether partial or total), whether detectable or undetectable. “Treating” and “treatment” can also mean prolonging survival as compared to expected survival if not receiving treatment. “Treating” and “treatment” as used herein also include prophylactic treatment. For example, a subject with early cancer can be treated to prevent progression, or alternatively a subject in remission can be treated with a compound or composition of the application to prevent recurrence. Treatment methods comprise administering to a subject a therapeutically effective amount of one or more of the compounds of the application and optionally consist of a single administration, or alternatively comprise a series of administrations.

“Palliating” a disease or disorder means that the extent and/or undesirable clinical manifestations of a disorder or a disease state are lessened and/or time course of the progression is slowed or lengthened, as compared to not treating the disorder.

The term “prevention” or “prophylaxis”, or synonym thereto, as used herein refers to a reduction in the risk or probability of a patient becoming afflicted with a disease, disorder or condition treatable by inhibiting HPK1, or manifesting a symptom associated with a disease, disorder or condition treatable by inhibition of HPK1.

As used herein, the term “effective amount” or “therapeutically effective amount” means an amount of a compound, or one or more compounds, of the application that is effective, at dosages and for periods of time necessary to achieve the desired result.

The expression “inhibiting HPK1” as used herein refers to inhibiting, blocking and/or disrupting HPK1 enzymatic activity in a cell, in particular a T-cell or B-cell. The inhibiting, blocking and/or disrupting causes a therapeutic effect in the cell.

By “inhibiting, blocking and/or disrupting” it is meant any detectable inhibition, block and/or disruption in the presence of a compound compared to otherwise the same conditions, except for in the absence in the compound.

The term “disease, disorder or condition treatable by inhibiting HPK1” means that the disease, disorder or condition to be treated is affected by, modulated by and/or has some biological basis, either direct or indirect, that includes HPK1 activity, in particular, increased HPK1 activity. These diseases respond favourably when HPK1 activity associated with the disease, disorder or condition is inhibited by one or more of the compounds or compositions of the application.

The term “HPK1” as used herein refers to the hematopoetic progenitor kinase 1.

The term “administered” as used herein means administration of a therapeutically effective amount of a compound, or one or more compounds, or a composition of the application to a cell either in cell culture or in a subject.

The term “neoplastic disorder” as used herein refers to a disease, disorder or condition characterized by cells that have the capacity for autonomous growth or replication, e.g., an abnormal state or condition characterized by proliferative cell growth. The term “neoplasm” as used herein refers to a mass of tissue resulting from the abnormal growth and/or division of cells in a subject having a neoplastic disorder. Neoplasms can be benign (such as uterine fibroids and melanocytic nevi), potentially malignant (such as carcinoma in situ) or malignant (i.e. cancer).

The term “cancer” as used herein refers to cellular-proliferative disease states.

As used herein, the term “effective amount” means an amount effective, at dosages and for periods of time, necessary to achieve a desired result.

### II. Compounds and Compositions

The present application describes a novel class of halo-substituted amino aza-heteroaryl HPK1 inhibitors.

Accordingly, the application includes a compound of Formula (I) or a pharmaceutically acceptable salt, solvate and/or prodrug thereof,

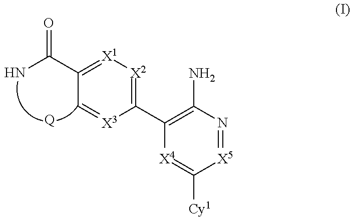

wherein:
X¹ is selected from N and CR¹;
X² and X³ are independently selected from N and CR²,
one of X⁴ and X⁵ is N and the other is CR³;
Q is C₁₋₄alkylene optionally interrupted by a heteroatom selected from O, S, S(O), SO₂, and NR⁴ and/or optionally substituted with one or more of R⁵ and/or optionally disubstituted on one carbon with R^{5a} and R^{5b}; provided that when Q comprises the heteromoiety the heteromoiety is not separated from the ring amide NH by methylene; or
Q is C₂₋₄alkenylene optionally substituted with one or more of R^{5c}; or
Q is optionally selected from C═N and N═C, and is optionally substituted with R^{5c};
R¹ is selected from H, halo, OR⁶, NR⁷R⁸, C₁₋₆alkyleneNR⁷R⁸ and C₁₋₆alkyl;
R² is selected from H, halo and C₁₋₆alkyl;
R³ is halo;
R⁴ is selected from H and C₁₋₆alkyl;
each R⁵ is independently selected from ═O, halo, C₁₋₆alkyl, C₃₋₆cycloalkyl, C₃₋₆heterocycloalkyl, C₁₋₆alkyleneC₃₋₆cycloalkyl, C₁₋₆alkyleneC₃₋₆heterocycloalkyl, OH, OC₁₋₆alkyl, NR⁹R¹⁰ and C₁₋₆alkyleneNR⁹R¹⁰;
R^{5a} and R^{5b} are joined to form, together with the carbon atom therebetween, a 3- to 6-membered, saturated or unsaturated ring optionally containing one heteromoiety selected from N, NH, NC₁₋₆alkyl, O, S, SO, and SO₂ and optionally substituted with one or more of halo and C₁₋₆alkyl;
each R^{5c} is independently selected from halo, C₁₋₆alkyl, C₃₋₆cycloalkyl, C₃₋₆heterocycloalkyl, C₁₋₆alkyleneC₃₋₆cycloalkyl, C₁₋₆alkyleneC₃₋₆heterocycloalkyl, OH, OC₁₋₆alkyl NR⁹R¹⁰, and C₁₋₆alkyleneNR⁹R¹⁰;
R⁶ is selected from H and C₁₋₆alkyl;
R⁷, R⁸, R⁹ and R¹⁰ are each independently selected from H and C₁₋₆alkyl; or
R⁷ and R⁸ or R⁹ and R¹⁰ are joined to form, together with the nitrogen atom therebetween, a 3- to 7-membered saturated or unsaturated ring, optionally containing one additional heteromoiety selected from N, NH, NC₁₋₆alkyl, O, S, S(O), and SO₂ and optionally substituted with one or more of halo and C₁₋₆alkyl;
Cy¹ is C₆₋₂₀aryl or C₅₋₂₀heteroaryl, and Cy¹ is unsubstituted or is substituted with one or more of R¹¹, or
Cy¹ is substituted with Z-Cy², or
Cy¹ is substituted with Z-Cy² and one or more of R¹¹;
each R¹¹ is independently selected from halo, ═O, CN, NO₂, C₁₋₆alkyl, C₂₋₆alkenyl, C₂₋₆alkynyl, OR¹², C(O)R¹², CO₂R¹², P(O)R¹²R¹³, P(O)(OR¹²)(OR¹³), SR¹², SOR¹², SO₂R¹², S(O)(═NR¹³)R¹², SO₂NR¹²R¹³, SiR¹⁴R^{14a}R^{14b}, C₁₋₆alkyleneOR¹², OC₁₋₆alkyleneOR¹², C₁₋₆alkyleneNR^{12a}R^{13a}, OC₁₋₆alkyleneNR¹²R¹³, NR^{13a}C₁₋₆alkyleneNR¹²R¹³, NR^{13a}C₁₋₆alkyleneOR¹², NR^{12a}R^{13a}, NR^{13a}COR¹², NR^{13a}CO₂R¹², NR^{13a}SO₂R¹², C₃₋₇cycloalkyl, C₃₋₇heterocycloalkyl, C₁₋₆alkyleneC₃₋₇cycloalkyl and C₁₋₆alkyleneC₃₋₇heterocycloalkyl, the latter four groups being optionally substituted with one or more of R¹⁵;
R¹² is selected from H, C₁₋₆alkyl, C₂₋₆alkenyl, C₂₋₆alkynyl, C₁₋₆alkyleneC₃₋₁₀cycloalkyl, C₁₋₆alkyleneC₃₋₁₀heterocycloalkyl, C₁₋₆alkyleneOR¹⁶, and C₁₋₆alkyleneNR^{16a}R^{16b}, and alkyl, alkenyl, alkynyl, alkylene, heterocycloalkyl and cycloalkyl groups of R¹² are optionally substituted with one or more of R¹⁷;
R¹³ is selected from H and C₁₋₆alkyl; or
R¹² and R¹³ are joined to form, together with the atom(s) therebetween, a 4- to 6-membered saturated or unsaturated ring, optionally containing one additional heteromoiety selected from N, NR¹⁸, O, S, SO, and SO₂, and optionally substituted with one or more of R¹⁷;
R^{12a} is selected from H, C₁₋₆alkyl, C₂₋₆alkenyl, C₂₋₆alkynyl, C₁₋₆alkyleneC₃₋₁₀cycloalkyl, C₁₋₆alkyleneC₃₋₁₀heterocycloalkyl, C₁₋₆alkyleneOR¹⁶, and C₁₋₆alkyleneNR^{16a}R^{16b}, and all alkyl, alkenyl, alkynyl, alkylene, heterocycloalkyl, and cycloalkyl groups of R^{12a} are optionally substituted with one or more of R¹⁷;
R^{13a} is selected from H and C₁₋₆alkyl;
R¹⁴, R^{14a}, and R^{14b} are independently selected from OR¹⁹, C₁₋₆alkyl, C₃₋₁₁cycloalkyl, C₃₋₁₀heterocycloalkyl, C₁₋₆alkyleneC₃₋₁₀cycloalkyl, and C₁₋₆alkyleneC₃₋₁₀heterocycloalkyl;
each R¹⁵ is independently selected from halo, OH, C₁₋₆alkyl, OC₁₋₆alkyl, CN and NR^{15a}R^{15b};
R^{15a} and R^{15b} are each independently selected from H and C₁₋₆alkyl;
R¹⁶, R^{16a} and R^{16b} are independently selected from H and C₁₋₆alkyl;
each R¹⁷ is independently selected from halo, C₁₋₆alkyl, CN and NR^{17a}R^{17b};
R^{17a} and R^{17b} are each independently selected from H and C₁₋₆alkyl;
R¹⁸ is selected from H and C₁₋₆alkyl;
R¹⁹ is selected from H, C₁₋₆alkyl, C₃₋₁₁cycloalkyl, C₃₋₁₀heterocycloalkyl, C₁₋₆alkyleneC₃₋₁₀cycloalkyl, C₁₋₆alkyleneC₃₋₁₀heterocycloalkyl, C₁₋₆alkyleneOR²⁰, and C₁₋₆alkyleneNR²⁰R²¹;
R²⁰ and R²¹ are independently selected from H and C₁₋₆alkyl;
Z is absent, or is selected from C₁₋₆alkylene, O, C(O), CO₂, S, S(O), SO₂, S(O)(═NR^{13b}) and NR^{13b}, C₁₋₆alkyleneO, C₁₋₆alkyleneC(O), C₁₋₆alkyleneCO₂, C₁₋₆alkyleneS, C₁₋₆alkyleneS(O), C₁₋₆alkyleneSO₂, C₁₋₆alkyleneS(O)(═NR^{13b}), C₁₋₆alkyleneNR^{13b}, OC₁₋₆alkylene, C(O)C₁₋₆alkylene, CO₂C₁₋₆alkylene, SC₁₋₆alkylene, S(O)C₁₋₆alkylene, SO₂C₁₋₆alkylene, S(O)(═NR^{13b})C₁₋₆alkylene and NR^{13b}C₁₋₆alkylene;
R^{13b} is selected from H and C₁₋₆alkyl;
Cy² is selected from C₃₋₁₄cycloalkyl and C₃₋₁₄heterocycloalkyl, and Cy² is unsubstituted or substituted with one or more of R²²;
each R²² is independently selected from halo, ═O, CN, OH, C₁₋₆alkyl, C₂₋₆alkenyl, C₂₋₆alkynyl, C₃₋₁₀cycloalkyl, C₃₋₁₀heterocycloalkyl, C₆₋₁₁aryl, C₅₋₁₄heteroaryl, C₁₋₆alkyleneC₃₋₁₀cycloalkyl, C₁₋₆alkyleneC₃₋₁₀heterocycloalkyl, C₁₋₆alkyleneC₆₋₁₁aryl, C₁₋₆alkyleneC₅₋₁₄heteroaryl, OC₁₋₆alkyl, OC₂₋₆alkenyl, OC₂₋₆alkynyl, C₁₋₆alkyleneOR²³, OC₁₋₆alkyleneOR²³, C₁₋₆alkyleneNR²⁴R²⁵, OC₁₋₆alkyleneNR²⁴R²⁵, SC₁₋₆alkyl, SC₂₋₆alkenyl, SC₂₋₆alkynyl, C(O)C₁₋₆alkyl, C(O)C₂₋₆alkenyl, C(O)C₂₋₆alkynyl, C(O)C₃₋₁₀cycloalkyl, C(O)C₃₋₁₀heterocycloalkyl, C(O)C₆₋₁₁aryl, C(O)C₅₋₁₄heteroaryl, C(O)C₁₋₆alkyleneC₃₋₁₀cycloalkyl, C(O)C₁₋₆alkyleneC₃₋₁₀heterocycloalkyl, C(O)C₁₋₆alkyleneC₆₋₁₁aryl, C(O)C₁₋₆alkyleneC₅₋₁₄heteroaryl, C(O)C₁₋₆alkylenylOR²³, C(O)C₁₋₆alkyleneNR²⁴R²⁵, C(O)C₁₋₆alkyleneOC₁₋₆alkyleneNR²⁴R²⁵, C(O)NR²⁴R²⁵, CO₂C₁₋₆alkyl, CO₂C₂₋₆alkenyl, CO₂C₂₋₆alkynyl, CO₂C₁₋₆alkyleneOR²³, CO₂C₁₋₆alkyleneOC₁₋₆alkyleneNR²⁴R²⁵, NR²⁴R²⁵, NR²⁶C₃₋₁₀cycloalkyl, NR²⁶C₃₋₁₀heterocycloalkyl, NR²⁶C₁₋₆alkyleneOR²³, NR²⁶C₁₋₆alkylene NR²⁴R²⁵, NR²⁶C₁₋₆alkyleneC₃₋₁₀cycloalkyl, NR²⁶C₁₋₆alkyleneC₃₋₁₀heterocycloalkyl, NR²⁶C₁₋₆alkyleneC₆₋₁₁aryl, NR²⁶C₁₋₆alkyleneC₅₋₁₄heteroaryl, NR²⁶SO₂C₁₋₆alkyl, SO₂C₁₋₆alkyl, SO₂C₂₋₆alkenyl, SO₂C₂₋₆alkynyl, and SO₂NR²⁴R²⁵, and alkyl, alkenyl, alkynyl, alkylene, aryl, heteroaryl, heterocycloalkyl, and cycloalkyl groups of R²² are optionally substituted with one or more of R²⁷;
R²³ is selected from H, C₁₋₆alkyl, C₁₋₆alkyleneOC₁₋₆alkyl, C₃₋₁₁cycloalkyl, C₃₋₁₀heterocycloalkyl, C₆₋₁₁aryl, C₅₋₁₄heteroaryl, C₂₋₆alkenyl, C₂₋₆alkynyl, C₁₋₆alkyleneC₃₋₁₀cycloalkyl, C₁₋₆alkyleneC₃₋₁₀heterocycloalkyl, C₁₋₆alkyleneC₆₋₁₁aryl, and C₁₋₆alkyleneC₅₋₁₄heteroaryl;
R²⁴ is selected from H and C₁₋₆alkyl;
R²⁵ is selected from H, C₁₋₆alkyl, C₁₋₆alkyleneOC₁₋₆alkyl, C₃₋₁₀cycloalkyl, C₃₋₁₀heterocycloalkyl, C₁₋₆-aryl, C₅₋₁₄heteroaryl, C₁₋₆alkyleneC₃₋₁₀cycloalkyl, C₁₋₆alkyleneC₃₋₁₀heterocycloalkyl, C₁₋₆alkyleneC₆₋₁₁aryl, and C₁₋₆alkyleneC₅₋₁₄heteroaryl, or
R²⁴ and R²⁵ are joined to form, together with the nitrogen atom therebetween, a 4- to 6-membered saturated or unsaturated ring, optionally containing one additional heteromoiety selected from N, NR²⁸, O, S, S(O), and SO₂, and optionally substituted with one or more of halo and C₁₋₆alkyl;
R²⁶ is selected from H and C₁₋₆alkyl;
each R²⁷ is independently selected from halo, C₁₋₆alkyl, CN and NR^{27a}R^{27b}; and
R^{27a}, R^{27b}, and R²⁸ are each independently selected from H and C₁₋₆alkyl;
wherein all available hydrogen atoms are optionally substituted with a fluorine atom.

The application also includes a compound of Formula (I) or a pharmaceutically acceptable salt, solvate and/or prodrug thereof,

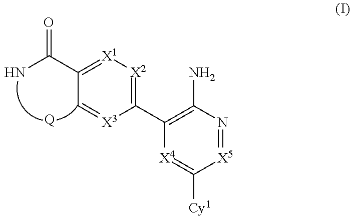

wherein:
X¹ is selected from N and CR¹;
X² and X³ are independently selected from N and CR²;
one of X⁴ and X⁵ is N and the other is CR³;
Q is C₁₋₄alkylene optionally interrupted by a heteroatom selected from O, S, S(O), SO₂, and NR⁴ and/or optionally substituted with one or more of R⁵ and/or optionally disubstituted on one carbon with R^{5a} and R^{5b}; provided that when Q comprises the heteromoiety the heteromoiety is not separated from the ring amide NH by methylene; or
Q is C₂₋₄alkenylene optionally substituted with one or more of R^{5c}; or
Q is optionally selected from C═N and N═C, and is optionally substituted with R^{5c};
R¹ is selected from H, halo, OR⁶, NR⁷R⁸, C₁₋₆alkyleneNR⁷R⁸ and C₁₋₆alkyl;
R² is selected from H, halo and C₁₋₆alkyl;
R³ is halo;
R⁴ is selected from H and C₁₋₆alkyl;
each R⁵ is independently selected from ═O, halo, C₁₋₆alkyl, C₃₋₆cycloalkyl, C₃₋₆heterocycloalkyl, C₁₋₆alkyleneC₃₋₆cycloalkyl, C₁₋₆alkyleneC₃₋₆heterocycloalkyl, OH, OC₁₋₆alkyl, NR⁹R¹⁰ and C₁₋₆alkyleneNR⁹R¹⁰;
R^{5a} and R^{5b} are joined to form, together with the atom therebetween, a 3- to 6-membered, saturated or unsaturated ring optionally containing one additional heteromoiety selected from N, NH, NC₁₋₆alkyl, O, S, SO, and SO₂ and optionally substituted with one or more of halo and C₁₋₆alkyl;
each R^{5c} is independently selected from halo, C₁₋₆alkyl, C₃₋₆cycloalkyl, C₃₋₆heterocycloalkyl, C₁₋₆alkyleneC₃₋₆cycloalkyl and C₁₋₆alkyleneC₃₋₆heterocycloalkyl, OH, OC₁₋₆alkyl NR⁹R¹⁰, and C₁₋₆alkyleneNR⁹R¹⁰;
R⁶ is selected from H and C₁₋₆alkyl;
R⁷ and R⁸ or R⁹ and R¹⁰ are each independently selected from H and C₁₋₆alkyl; or
R⁷ and R⁸ or R⁹ and R¹⁰ are joined to form, together with the atom therebetween, a 3- to 7-membered saturated or unsaturated ring, optionally containing one additional heteromoiety selected from N, NH, NC₁₋₆alkyl, O, S, S(O), and SO₂ and optionally substituted with one or more of halo and C₁₋₆alkyl;
Cy¹ is C₆₋₂₀aryl or C₅₋₂₀heteroaryl, and Cy¹ is unsubstituted or is substituted with one or more of R¹¹, or
Cy¹ is substituted with Z-Cy², or
Cy¹ is substituted with Z-Cy² and one or more of R¹¹;
each R¹¹ is independently selected from halo, ═O, CN, NO₂, C₁₋₆alkyl, C₂₋₆alkenyl, C₂₋₆alkynyl, OR¹², C(O)R¹², CO₂R¹², P(O)R¹²R¹³, P(O)(OR¹²)(OR¹³), SR¹², SOR¹², SO₂R¹², S(O)(═NR¹³)R¹², SO₂NR¹²R¹³, SiR¹⁴R^{14a}R^{14b}, C₁₋₆alkyleneOR¹², OC₁₋₆alkyleneOR¹², C₁₋₆alkyleneNR^{12a}R^{13a}, OC₁₋₆alkyleneNR¹²R¹³, NR^{13a}C₁₋₆alkyleneNR¹²R¹³, NR^{13a}C₁₋₆alkyleneOR¹², NR^{12a}R^{13a}, NR^{13a}COR¹², NR^{13a}CO₂R¹², NR^{13a}SO₂R¹², C₃₋₇cycloalkyl, C₃₋₇heterocycloalkyl, C₁₋₆alkyleneC₃₋₇cycloalkyl and C₁₋₆alkyleneC₃₋₇heterocycloalkyl, the latter four groups being optionally substituted with one or more of R¹⁵;
R¹² is selected from H, C₁₋₆alkyl, C₂₋₆alkenyl, C₂₋₆alkynyl, C₁₋₆alkyleneC₃₋₁₀cycloalkyl, C₁₋₆alkyleneC₃₋₁₀heterocycloalkyl, C₁₋₆alkyleneOR¹⁷, and C₁₋₆alkyleneNR^{16a}R^{16b}, and alkyl, alkenyl, alkynyl, alkylene and cycloalkyl groups of R¹² are optionally substituted with one or more of R¹⁷;
R¹³ is selected from H and C₁₋₆alkyl; or
R¹² and R¹³ are joined to form, together with the atom therebetween, a 4- to 6-membered saturated or unsaturated ring, optionally containing one additional heteromoiety selected from N, NR¹⁶, O, S, SO, and SO₂, and optionally substituted with one or more of R¹⁷;
R^{12a} is selected from H, C₁₋₆alkyl, C₂₋₆alkenyl, C₂₋₆alkynyl, C₁₋₆alkyleneC₃₋₁₀cycloalkyl, C₁₋₆alkyleneC₃₋₁₀heterocycloalkyl, C₁₋₆alkyleneOR¹⁴, and C₁₋₆alkyleneNR¹¹R¹⁴, and all alkyl, alkenyl, alkynyl, alkylene, and cycloalkyl groups of R^{10a} are optionally substituted with one or more of R¹⁷;
R^{13a} is selected from H and C₁₋₆alkyl;
R¹⁴, R^{14a}, and R^{14b} are independently selected from OR¹⁹, C₁₋₆alkyl, C₃₋₁₁cycloalkyl, C₃₋₁₀heterocycloalkyl, C₁₋₆alkyleneC₃₋₁₀cycloalkyl, and C₁₋₆alkyleneC₃₋₁₀heterocycloalkyl;
each R¹⁵ is independently selected from halo, C₁₋₆alkyl, CN and NR^{15a}R^{15b};
R^{15a} and R^{15b} are each independently selected from H and C₁₋₆alkyl;
R^{16a} and R^{16b} are independently selected from H and C₁₋₆alkyl;
each R¹⁷ is independently selected from halo, C₁₋₆alkyl, CN and NR^{17a}R^{17b};
R^{17a} and R^{17b} are each independently selected from H and C₁₋₆alkyl;
R¹⁸ is selected from H and C₁₋₆alkyl;
R¹⁹ is selected from H, C₁₋₆alkyl, C₃₋₁₁cycloalkyl, C₃₋₁₀heterocycloalkyl, C₁₋₆alkyleneC₃₋₁₀cycloalkyl, C₁₋₆alkyleneC₃₋₁₀heterocycloalkyl C₁₋₆alkyleneOR²⁰, and C₁₋₆alkyleneNR²⁰R²¹;
R²⁰ and R²¹ are independently selected from H and C₁₋₆alkyl;
Z is absent, or is selected from C₁₋₆alkylene, O, C(O), CO₂, S, S(O), SO₂, S(O)(═NR^{13b}) and NR^{13b};
R^{13b} is selected from H and C₁₋₆alkyl;
Cy² is selected from C₃₋₁₄cycloalkyl and C₃₋₁₄heterocycloalkyl, and Cy² is unsubstituted or substituted with one or more of R²²;
each R²² is independently selected from halo, ═O, CN, OH, C₁₋₆alkyl, C₂₋₆alkenyl, C₂₋₆alkynyl, C₃₋₁₀cycloalkyl, C₃₋₁₀heterocycloalkyl, C₁₋₆alkyleneC₃₋₁₀cycloalkyl, C₁₋₆alkyleneC₃₋₁₀heterocycloalkyl, OC₁₋₆alkyl, OC₂₋₆alkenyl, OC₂₋₆alkynyl, C₁₋₆alkyleneOR²³, OC₁₋₆alkyleneOR²³, C₁₋₆alkyleneNR²⁴R²⁵, OC₁₋₆alkyleneNR²⁴R²⁵, SC₁₋₆alkyl, SC₂₋₆alkenyl, SC₂₋₆alkynyl, C(O)C₁₋₆alkyl, C(O)C₂₋₆alkenyl, C(O)C₂₋₆alkynyl, C(O)C₃₋₁₀cycloalkyl, C(O)C₃₋₁₀heterocycloalkyl, C(O)C₁₋₆alkyleneC₃₋₁₀cycloalkyl, C(O)C₁₋₆alkyleneC₃₋₁₀heterocycloalkyl, C(O)C₁₋₆alkylenylOR²³, C(O)C₁₋₆alkyleneNR²⁴R²⁵, C(O)C₁₋₆alkyleneOC₁₋₆alkyleneNR²⁴R²⁵, C(O)NR²⁴R²⁵, CO₂C₁₋₆alkyl, CO₂C₂₋₆alkenyl, CO₂C₂₋₆alkynyl, CO₂C₁₋₆alkyleneOR²³, CO₂C₁₋₆alkyleneOC₁₋₆alkyleneNR²⁴R²⁵, NR²⁴R²⁵, NR²⁶C₃₋₁₀cycloalkyl, NR²⁶C₃₋₁₀heterocycloalkyl, NR²⁶C₁₋₆alkyleneOR²³, NR²⁶C₁₋₆alkyleneC₃₋₁₀cycloalkyl, NR²⁶C₁₋₆alkyleneC₃₋₁₀heterocycloalkyl, NR²⁶SO₂C₁₋₆alkyl, SO₂C₁₋₆alkyl, SO₂C₂₋₆alkenyl, SO₂C₂₋₆alkynyl, and SO₂NR²⁴R²⁵, and alkyl, alkenyl, alkynyl, alkylene and cycloalkyl groups of R²² are optionally substituted with one or more of R²⁷;
R²³ is selected from H, C₁₋₆alkyl, C₁₋₆alkyleneOC₁₋₆alkyl, C₃₋₁₁cycloalkyl, C₃₋₁₀heterocycloalkyl, C₂₋₆alkenyl, C₂₋₆alkynyl, C₁₋₆alkyleneC₃₋₁₀cycloalkyl, and C₁₋₆alkyleneC₃₋₁₀heterocycloalkyl;
R²⁴ is selected from H and C₁₋₆alkyl;
R²⁵ is selected from H, C₁₋₆alkyl, C₁₋₆alkyleneOC₁₋₆alkyl, C₃₋₁₀cycloalkyl, C₃₋₁₀heterocycloalkyl, C₁₋₆alkyleneC₃₋₁₀cycloalkyl, and C₁₋₆alkyleneC₃₋₁₀heterocycloalkyl, or
R²⁴ and R²⁵ are joined to form, together with the atom therebetween, a 4- to 6-membered saturated or unsaturated ring, optionally containing one additional heteromoiety selected from N, NR²⁸, O, S, S(O), and SO₂, and optionally substituted with one or more of halo and C₁₋₆alkyl;
R²⁶ is selected from H and C₁₋₆alkyl;
each R²⁷ is independently selected from halo, C₁₋₆alkyl, CN and NR^{27a}R^{27b}; and
R^{27a}, R^{27b}, and R²⁸ are each independently selected from H and C₁₋₆alkyl;
wherein all available hydrogen atoms are optionally substituted with a fluorine atom.

In all embodiments below it is to be understood that all available hydrogen atoms are optionally substituted with a fluorine atom. This has not been repeated throughout. Thus in each embodiment where a group is listed that comprises available hydrogen atoms, it is to be understood that all such atoms are optionally replaced with fluorine atoms, for example each recitation of C₁₋₆alkyl is also a recitation of C₁₋₆fluoroalkyl, unless stated otherwise.

In an embodiment, X¹ is N.

In an embodiment, X¹ is CR¹.

In an embodiment, R¹ is selected from H, F, Cl, OR⁶, NR⁷R⁸, C₁₋₄alkyleneNR⁷R⁸ and C₁₋₄alkyl. In an embodiment, R¹ is selected from H, F, Cl and C₁₋₄alkyl. In an embodiment, R¹ is selected from H, F, Cl, CH₃, CH₂CH₃, CF₂H, CF₃, CFH₂, CH₂CF₂H and CH₂CF₂H. In an embodiment, R¹ is selected from H, F, CF₃, CF₂H, CH₂CF₂H and CH₃. In an embodiment, R¹ is selected from H, F, CF₃, CF₂H and CH₂CF₂H. In an embodiment, R¹ is selected from H, F, CF₃ and CH₃. In an embodiment, R¹ is selected from H and F. In an embodiment, R¹ is F. In an embodiment, R¹ is H.

In an embodiment, R¹ is OR⁶. In an embodiment, R⁶ is selected from H, CH₃, CH₂CH₃, CF₃, CFH₂, CF₂H, CH₂CF₂H, and CH₂CF₂H. In an embodiment, R⁶ is selected from H and C₁₋₄alkyl. In an embodiment, R⁶ is selected from H, CH₃, CH₂CH₃, CF₃, CFH₂, CF₂H, CH₂CF₂H, and CH₂CH₂F. In an embodiment, R⁶ is CF₂H. Accordingly, in an embodiment, R¹ is selected from OH, OCH₃, OCH₂CH₃, OCF₃, OCFH₂, OCHF₂, OCH₂CF₂H, and OCH₂CF₂H.

In an embodiment, R¹ is selected from NR⁷R⁸ and C₁₋₄alkyleneNR⁷R⁸. In an embodiment, R⁷ and R⁸ are each independently selected from H and C₁₋₄alkyl. In an embodiment, R¹ is selected from NR⁷R⁸ and C₁₋₂alkyleneNR⁷R⁸.

In an embodiment, R⁷ and R⁸ are each independently selected from H and C₁₋₄alkyl. In an embodiment, R⁷ and R⁸ are each independently selected from H, CH₃ and CF₃ In an embodiment, one of R⁷ and R⁸ is H and the other is CH₃. In an embodiment, R⁷ and R⁸ are both CH₃. In an embodiment, R⁷ and R⁸ are both H.

In an embodiment, R⁷ and R⁸ are joined to form, together with the nitrogen atom therebetween, a 3- to 7-membered saturated or unsaturated ring optionally containing one additional heteromoiety selected from N, NH, NC₁₋₆alkyl, O, S, S(O), and SO₂, and optionally substituted with one or more of halo and C₁₋₆alkyl. In an embodiment, R⁷ and R⁸ are joined to form, together with the nitrogen atom therebetween to form a 3- to 7-membered heterocyclic ring selected from azetidinyl, diazetidinyl, pyrrolidinyl, imidazolidinyl, pyrazolidinyl, thiazolidinyl, isothiozolidinyl, piperidinyl, diazinanyl (e.g. piperazinyl), morpholinyl and azepanyl, and optionally substituted with one or more of halo and C₁₋₆alkyl. In an embodiment, R⁷ and R⁸ are joined to form, together with the nitrogen atom therebetween, a 4- to 6-membered saturated ring, and optionally substituted with one or more of halo and C₁₋₆alkyl. In an embodiment, R⁷ and R⁸ are joined to form, together with the nitrogen atom therebetween, aziridinyl, azetidinyl, pyrrolidinyl, or piperidinyl, and optionally substituted with one or more of halo and C₁₋₆alkyl.

In an embodiment, one of X² and X³ is N and the other is CR². In an embodiment, X² is N and X³ is CR². In an embodiment, both X² and X³ are, independently, CR². In an embodiment, each R² is independently selected from H, halo and C₁₋₄alkyl. In an embodiment, each R² is independently selected from H, F, C and C₁₋₄alkyl. In an embodiment, each R² is independently selected from H, F, C, CH₃, CF₃, CH₂F and CHF₂. In an embodiment, each R² is independently selected from H, F, C, CH₃ and CF₃. In an embodiment, each R² is selected from H and F. In an embodiment, one of X² and X³ is N and the other is CH. In an embodiment, X² is N and X³ is CH. In an embodiment, X² is selected from CH, CF, CCl, CCH₃ and CCF₃ and X³ is CH. In an embodiment, X² is CF or CCl and X³ is CH. In an embodiment, X² is CH and X³ is selected from CH, CF, CCl, CCH₃ and CCF₃. In an embodiment, one of X² and X³ is CF and the other is CH. In an embodiment, X² is CF and X³ is CH. In an embodiment, X² and X³ are both CF. In an embodiment, X² and X³ are both CH. In an embodiment, both X² and X³ are N.

In an embodiment, X¹ is CR¹ and X² and X³ are both, independently, CR⁴. In an embodiment, X¹ is N and an X¹ and X² are both, independently, CR⁴. In an embodiment, X¹ is N and an X¹ and X² are both CH. In an embodiment, X¹ is CR¹ and one of X¹ and X² is N and the other is CR⁴. In an embodiment, X¹ is CR¹ and one of X¹ and X² is N and the other is CH. In an embodiment, X¹ is CR¹ and both X² and X³ are N.

In an embodiment, X⁴ is N and X⁵ is CR³. In an embodiment, X⁵ is N and X⁴ is CR³.

In an embodiment, R³ is C, F or Br. In an embodiment, R³ is C or F. In an embodiment, R³ is F.

In an embodiment, X⁴ is N and X⁵ is CF.

In an embodiment, Q is C₁₋₃alkylene optionally interrupted by a heteromoiety selected from O, S, S(O), SO₂, and NR² and/or optionally substituted with one or more of R⁵.

In an embodiment, Q is C₁₋₃alkylene optionally interrupted by a heteromoiety selected from O, S, S(O), SO₂, and NR⁴. In an embodiment, Q is C₁₋₃alkylene optionally interrupted by a heteromoiety selected from O, SO₂, and NR⁴. In an embodiment, Q is C₁₋₃alkylene optionally interrupted by O or NR⁴. In an embodiment, Q is C₁₋₃alkylene optionally interrupted by O.

In an embodiment, R⁴ is selected from H and C₁₋₄alkyl. In an embodiment, R⁴ is selected from CH₃, CH₂CH₃, CH(CH₃)₂, C(CH₃)₄ and CH₂CH(CH₃)₂. In an embodiment, R⁴ is selected from CH₃, CH₂CH₃, CF₂H, CF₃, CFH₂, CH₂CF₂H, and CH₂CF₃. In an embodiment, R⁴ is selected from CF₂H, CH₃ and CF₃. In an embodiment, R⁴ is selected from CH₃ and CF₃.

In an embodiment, Q is C₁₋₃alkylene and optionally substituted with one to three of R⁵. In an embodiment, Q is CH₂ or CH₂CH₂ and optionally substituted with one or two of R⁵. In an embodiment, Q is C₁alkylene and optionally substituted with one or two of R⁵. In an embodiment, Q is CH₂. In an embodiment, Q is CH₂CH₂.

In an embodiment, each R⁵ is independently selected from ═O, F, C, C₁₋₄alkyl, C₃₋₆cycloalkyl, C₃₋₆heterocycloalkyl, C₁₋₆alkyleneC₃₋₆cycloalkyl, C₁₋₆alkyleneC₃₋₆heterocycloalkyl, OH, OC₁₋₆alkyl, NR⁹R¹⁰, and C₁₋₆alkyleneNR⁹R¹⁰. In an embodiment, each R⁵ is independently selected from ═O, F, Cl, C₁₋₄alkyl, C₃₋₆cycloalkyl, C₃₋₆heterocycloalkyl, C₁₋₄alkyleneC₃₋₆cycloalkyl, C₁₋₄alkyleneC₃₋₆heterocycloalkyl, OH, OC₁₋₄alkyl, NR⁹R¹⁰, and C₁₋₄alkyleneNR⁹R¹⁰. In an embodiment, Q is substituted with one R⁵ and R⁵ is ═O.

In an embodiment, each R⁵ is independently selected from F, Cl, OH, C₁₋₄alkyl OC₁₋₄alkyl and NR⁹R¹⁰. In an embodiment, each R⁵ is independently selected from F, Cl, OH, CH₃, CH₂CH₃, CF₂H, CF₃, CFH₂, CH₂CF₂H, OCH₃, OCH₂CH₃, OCF₃, OCF₂H, OCH(CH₃)₂ and NR⁹R¹⁰. In an embodiment, each R⁵ is independently selected from F, Cl, OH, CH₃, CF₂H, CF₃, CFH₂, OCH₃, OCF₃, OCF₂H and NR⁹R¹⁰. In an embodiment, one to three of R⁵ are independently selected from F, Cl, CH₃, CF₂H, CF₃, OCH₃, OCF₃, OCF₂H and NR⁹R¹⁰. In an embodiment, one to three of R⁵ are independently selected from F, Cl, CH₃, CF₂H, CF₃, OCH₃, OCF₃ and OCF₂H. In an embodiment, one to four of R⁵ are independently selected from F, CH₃, and OCH₃.

In an embodiment, each R⁵ is independently selected from F, C and C₁₋₄alkyl. In an embodiment, each R⁵ is independently selected from F, C, CH₃, CH₂CH₃, CF₂H, CF₃, CFH₂, CH₂CF₂H, and CH₂CH₂F. In an embodiment, each R⁵ is independently selected from F, C, CH₃, CF₂H, CF₃ and CH₂CF₂H. In an embodiment, each R⁵ is independently selected from F, C, CH₃, and CF₃. In an embodiment, each R⁵ is independently selected from F, CH₃, and CF₃ In an embodiment, each R⁵ is independently selected from F, CH₃, and CF₃. In an embodiment, each R⁵ is independently selected from F and CH₃. In an embodiment, at least one R⁵ is F. In an embodiment, one or two of R⁵ is F. In an embodiment, one or more, one to four, one to three, one or two, or one of R⁵ is CH₃.

In an embodiment, one or two of R⁵ are independently selected from C₃₋₆cycloalkyl, C₃₋₆heterocycloalkyl, C₁₋₄alkyleneC₃₋₆cycloalkyl and C₁₋₄alkyleneC₃₋₆heterocycloalkyl. In an embodiment, one R⁵ is selected from C₃₋₆cycloalkyl, C₃₋₆heterocycloalkyl, C₁₋₂alkyleneC₃₋₆cycloalkyl and C₁₋₂alkyleneC₃₋₆heterocycloalkyl. In an embodiment, the cycloalkyl in R⁵ is selected from cyclopropyl, cyclobutyl, cyclopentyl and cyclohexyl. In an embodiment, the cycloalkyl in R⁵ is selected from cyclopropyl and cyclobutyl.

In an embodiment, the heterocycloalkyl in R⁵ is selected from aziridinyl, oxiranyl, thiiranyl, oxaxiridinyl, dioxiranyl, azetidinyl, oxetanyl, thietanyl, diazetidinyl, dioxetanyl, dithietanyl, tetrahydrofuranyl, tetrahydrothiophenyl, pyrrolidinyl, imidazolidinyl, pyrazolidinyl, isoxthiolidinyl, thiazolidinyl, isothiazolidinyl, dioxolanyl, dithiolanyl, piperidinyl, triazolyl, furazanyl, oxadiazolyl, thiadiazolyl, dioxazolyl, dithiazolyl, tetrazolyl, oxatetrazolyl, tetrahydropyranyl, diazinanyl (e.g, piperazinyl), morpholinyl, thiomorpholinyl, dioxanyl, and dithianyl. In an embodiment, the heterocycloalkyl in R⁵ is selected from azetidinyl, oxetanyl, thietanyl, tetrahydrofuranyl, pyrrolidinyl, imidazolidiny and pyrazolidinyl.

In an embodiment, each R⁵ is independently selected from OH and OC₁₋₄alkyl. In an embodiment, each R⁵ is independently selected from OH and OC₁₋₄alkyl. In an embodiment, each R⁵ is independently selected from OH, OCH₃, OCF₃, OCF₂H, OCH₂CH₃ and OCH(CH₃)₂. In an embodiment, one or two of R⁵ are independently selected from OH, OCH₃, OCF₃, and OCF₂H.

In an embodiment, one R⁵ is selected from NR⁹R¹⁰ and C₁₋₄alkyleneNR⁹R¹⁰. In an embodiment, one of R⁵ is NR⁹R¹⁰ or C₁₋₂alkyleneNR⁹R¹⁰. In an embodiment, one of R⁵ is NR⁹R¹⁰. In an embodiment, one of R⁵ is C₁₋₄alkyleneNR⁹R¹⁰. In an embodiment, the R⁹ and R¹⁰ in NR⁹R¹⁰ or C₁₋₂alkyleneNR⁹R¹⁰ of R⁵ are both CH₃ or are both H. In an embodiment, one R⁵ is NR⁹R¹⁰ and R⁹ and R¹⁰ in R⁵ are both H.

In an embodiment, R⁹ and R¹⁰ in R⁵ are each independently selected from H and C₁₋₄alkyl. In an embodiment, R⁹ and R¹⁰ in R⁵ are each independently selected from H, CH₃ and CF₃ In an embodiment, one of R⁹ and R¹⁰ in R⁵ is H and the other is CH₃. In an embodiment, R⁹ and R¹⁰ in R⁵ are both CH₃. In an embodiment, one R⁵ is NR⁹R¹⁰ and R⁹ and R¹⁰ in R⁵ are both CH₃ or both H. In an embodiment, one R⁵ is NR⁹R¹⁰ and R⁹ and R¹⁰ in R⁶ are both H.

In an embodiment, R⁹ and R¹⁰ are joined to form, together with the nitrogen atom therebetween, a 3- to 7-membered saturated or unsaturated ring optionally containing one additional heteromoiety selected from N, NH, NC₁₋₆alkyl, O, S, S(O), and SO₂, and optionally substituted with one or more of halo and C₁₋₆alkyl. In an embodiment, R⁹ and R¹⁰ are taken together with the nitrogen atom therebetween to form a 3- to 7-membered heterocyclic ring selected from azetidinyl, diazetidinyl, pyrrolidinyl, imidazolidinyl, pyrazolidinyl, thiazolidinyl, isothiozolidinyl, piperidinyl, diazinanyl (e.g. piperazinyl), morpholinyl and azepanyl, optionally substituted with one to four of halo and C₁₋₆alkyl. In an embodiment, R⁹ and R¹⁰ are joined to form, together with the nitrogen atom therebetween, a 4 to 6 heterocycloalkyl ring, optionally substituted with one to four of halo and C₁₋₄alkyl. In an embodiment R⁹ and R¹⁰ are joined to form, together with the nitrogen atom therebetween, aziridinyl, azetidinyl, pyrrolidinyl or piperidinyl, optionally substituted with one to four of C, F and C₁₋₄alkyl.

In an embodiment, Q is unsubstituted. In an embodiment, Q is C₁₋₃alkylene and is substituted with one or two of R⁵. In an embodiment, Q is C₁₋₃alkylene and is substituted with one or two of R⁵, and R⁵ is C₁₋₄alkyl. In an embodiment, Q is C₁₋₃alkylene and is substituted with one or two of R⁵, and R⁵ is CH₃. In an embodiment, Q is C₁₋₃alkylene and is substituted with one to four of R⁵, and each R⁵ is independently selected from F, Cl, CH₃, CF₂H, CF₃, OCH₃, OCF₃ and OCF₂H. In an embodiment, Q is C₁₋₃alkylene and is substituted with one to four of R⁵, and each R⁵ is independently selected from F, CH₃ and OCH₃.

In an embodiment, Q is C₁₋₃alkylene and optionally disubstituted on one carbon atom with R^{5a} and R^{5b}. In an embodiment, Q is C₁alkylene or C₂alkylene, and optionally disubstituted on one carbon atom with R^{5a} and R^{5b}. In an embodiment, Q is CR^{5a}R^{5b}.

In an embodiment, R^{5a} and R^{5b} are joined to form, together with the carbon atom therebetween, a 3- to 6-membered saturated or unsaturated ring optionally containing one heteromoiety selected from N, NH, NC₁₋₆alkyl, O, S, S(O), and SO₂ and optionally substituted with one or more of halo and C₁₋₄alkyl.

In an embodiment, R^{5a} and R^{5b} are joined to form, together with the carbon atom therebetween, a 3- to 6-membered cycloalkyl ring and optionally substituted with one or more of halo and C₁₋₄alkyl. In an embodiment, R^{5a} and R^{5b} are joined to form, together with the carbon atom therebetween, to form a 3- to 6-membered cycloalkyl selected from cyclopropyl, cyclobutyl, cyclopentyl and cyclohexyl and optionally substituted with one or more of halo and C₁₋₄alkyl. In an embodiment, R^{5a} and R^{5b} are joined to form, together with the carbon atom therebetween, a 3- to 5-membered cycloalkyl ring and optionally substituted with one to four of halo and C₁₋₄alkyl. In an embodiment, R^{5a} and R^{5b} are joined to form, together with the carbon atom therebetween, a cyclopropyl, a cyclobutyl or a cyclopentyl ring and optionally substituted with one to four of halo and C₁₋₄alkyl. In an embodiment, R^{5a} and R^{5b} are joined to form, together with the carbon atom therebetween, a cyclopropyl or a cyclobutyl ring and optionally substituted with one to three of halo and C₁₋₄alkyl. In an embodiment, R^{5a} and R^{5b} are joined to form, together with the carbon atom therebetween, a cyclopropyl ring and optionally substituted with one to three of C, F and C₁₋₄alkyl. In an embodiment, R^{5a} and R^{5b} are joined to form, together with the carbon atom therebetween, a cyclopropyl or a cyclobutyl ring. In an embodiment, R^{5a} and R^{5b} are joined to form, together with the carbon atom therebetween, a cyclopropyl ring.

In an embodiment, R^{5a} and R^{5b} are joined to form, together with the carbon atom therebetween, a 3- to 6-membered heterocycloalkyl ring, and optionally substituted with one or more of halo and C₁₋₄alkyl. In an embodiment, R^{5a} and R^{5b} are joined to form, together with the carbon atom therebetween, a 3- to 6-membered heterocycloalkyl ring selected from aziridinyl, oxiranyl, thiiranyl, oxaxiridinyl, dioxiranyl, azetidinyl, oxetanyl, thietanyl, diazetidinyl, dioxetanyl, dithietanyl, tetrahydrofuranyl, tetrahydrothiophenyl, pyrrolidinyl, imidazolidinyl, pyrazolidinyl, isoxthiolidinyl, thiazolidinyl, isothiazolidinyl, dioxolanyl, dithiolanyl, piperidinyl, triazolyl, furazanyl, oxadiazolyl, thiadiazolyl, dioxazolyl, dithiazolyl, tetrazolyl, oxatetrazolyl, tetrahydropyranyl, diazinanyl (e.g, piperazinyl), morpholinyl, thiomorpholinyl, dioxanyl and dithianyl. In an embodiment, R^{5a} and R^{5b} are joined to form, together with the carbon atom therebetween, a 3- to 5-membered heterocycloalkyl ring. In an embodiment, R^{5a} and R^{5b} are joined to form, together with the carbon atom therebetween, a 3- to 5-membered heterocycloalkyl ring selected from oxiranyl, oxetanyl, azetidinyl, thietanyl, pyrrolidinyl, tetrahydrofuranyl, and tetrahydrothiophenyl ring. In an embodiment, R^{5a} and R^{5b} are joined to form, together with the carbon atom therebetween, an oxetanyl, or an azetidinyl ring. In an embodiment, R^{5a} and R^{5b} are joined to form, together with the atom therebetween, an oxetanyl ring. In an embodiment, R^{5a} and R^{5b} are joined to form, together with the carbon atom therebetween to form

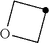

wherein “•” indicates a point of attachment to Q.

In an embodiment, Q is C₂₋₄alkenylene optionally substituted with one or two of R^{5c}. In an embodiment, Q is —C═C— optionally substituted with one or more (e.g. one or two) of R^{5c}.

In an embodiment, Q is selected from C═N and N═C, and is optionally substituted with R^{5c}. In an embodiment, Q is C═N or N═C.

In an embodiment, each R^{5c} is independently selected from F, Cl, C₁₋₄alkyl, C₃₋₆cycloalkyl, C₃₋₆heterocycloalkyl, C₁₋₆alkyleneC₃₋₆cycloalkyl, C₁₋₆alkyleneC₃₋₆heterocycloalkyl, OH, OC₁₋₆alkyl, NR⁹R¹⁰, and C₁₋₆alkyleneNR⁹R¹⁰. In an embodiment, each R^{5c} is independently selected from F, Cl, C₁₋₄alkyl, C₃₋₆cycloalkyl, C₃₋₆heterocycloalkyl, C₁₋₄alkyleneC₃₋₆cycloalkyl, C₁₋₄alkyleneC₃₋₆heterocycloalkyl, OH, OC₁₋₄alkyl, NR⁹R¹⁰, and C₁₋₄ alkyleneNR⁹R¹⁰.

In an embodiment, each R^{5c} is independently selected from F, Cl, OH, C₁₋₄alkyl, OC₁₋₄alkyl, C₁₋₂alkyleneNR⁹R¹⁰ and NR⁹R¹⁰. In an embodiment, each R^{5c} is independently selected from F, Cl, OH, CH₃, CH₂CH₃, CF₂H, CF₃, CFH₂, CH₂CF₂H, OCH₃, OCH₂CH₃, OCF₃, OCF₂H, OCH(CH₃)₂, C₁₋₂alkyleneNR⁹R¹⁰ and NR⁹R¹⁰. In an embodiment, each R^{5c} is independently selected from F, Cl, OH, CH₃, CF₂H, CF₃, CFH₂, OCH₃, OCF₃, OCF₂H, C₁₋₂alkyleneR⁹R¹⁰ and NR⁹R¹⁰. In an embodiment, each R^{5c} is independently selected from F, Cl, CH₃, CF₂H, CF₃, OCH₃, OCF₃, OCF₂H, C₁₋₂alkyleneNR⁹R¹⁰ and NR⁹R¹⁰. In an embodiment, each R^{5c} is independently selected from F, Cl, CH₃, CF₂H, CF₃, OCH₃, OCF₃, OCF₂H, C₁₋₂alkyleneR⁹R¹⁰ and NR⁹R¹⁰.

In an embodiment, each R^{5c} is independently selected from F and C₁₋₄alkyl. In an embodiment, each R^{5c} is independently selected from F, Cl, CH₃, CH₂CH₃, CF₂H, CF₃, CFH₂, CH₂CF₂H, and CH₂CH₂F. In an embodiment, each R^{5c} is independently selected from F, C, CH₃, CF₂H, CF₃ and CH₂CF₂H. In an embodiment, each R^{5c} is independently selected from F, C, CH₃, and CF₃. In an embodiment, R^{5c} is selected from F, CH₃, and CF₃. In an embodiment, each R^{5c} is selected from F and CH₃. In an embodiment, at least one R^{5c} is F. In an embodiment, one or more, one to four, one to three, one or two, or one of R^{5c} is CH₃. In an embodiment, each R^{5c} is CH₃. In an embodiment, one or two of R^{5c} are independently selected from C₃₋₆cycloalkyl, C₃₋₆heterocycloalkyl, C₁₋₄alkyleneC₃₋₆cycloalkyl and C₁₋₄alkyleneC₃₋₆heterocycloalkyl. In an embodiment, one R^{5c} is selected from C₃₋₆cycloalkyl, C₃₋₆heterocycloalkyl, C₁₋₂alkyleneC₃₋₆cycloalkyl and C₁₋₂alkyleneC₃₋₆heterocycloalkyl. In an embodiment, the cycloalkyl in R^{5c} is selected from cyclopropyl, cyclobutyl, cyclopentyl and cyclohexyl. In an embodiment, the cycloalkyl in R^{5c} is selected from cyclopropyl and cyclobutyl.

In an embodiment, the heterocycloalkyl R^{5c} is selected from aziridinyl, oxiranyl, thiiranyl, oxaxiridinyl, dioxiranyl, azetidinyl, oxetanyl, thietanyl, diazetidinyl, dioxetanyl, dithietanyl, tetrahydrofuranyl, tetrahydrothiophenyl, pyrrolidinyl, imidazolidinyl, pyrazolidinyl, isoxthiolidinyl, thiazolidinyl, isothiazolidinyl, dioxolanyl, dithiolanyl, piperidinyl, triazolyl, furazanyl, oxadiazolyl, thiadiazolyl, dioxazolyl, dithiazolyl, tetrazolyl, oxatetrazolyl, tetrahydropyranyl, diazinanyl (e.g, piperazinyl), morpholinyl, thiomorpholinyl, dioxanyl, and dithianyl. In an embodiment, the heterocycloalkyl in R^{5c} is selected from azetidinyl, oxetanyl, thietanyl, tetrahydrofuranyl, pyrrolidinyl, imidazolidiny and pyrazolidinyl.

In an embodiment, each R^{5c} is independently selected from OH and OC₁₋₄alkyl. In an embodiment, each R^{5c} is independently selected from OH and OC₁₋₄alkyl. In an embodiment, each R^{5c} is independently selected from OH, OCH₃, OCF₃, OCF₂H, OCH₂CH₃ and OCH(CH₃)₂. In an embodiment, one R^{5c} is selected from OH, OCH₃, OCF₃, and OCF₂H.

In an embodiment, one or two of R^{5c} is C₁₋₄alkyleneNR⁹R¹⁰. In an embodiment, one R^{5c} is C₁₋₂alkyleneNR⁹R¹⁰. In an embodiment, one of R^{5c} is NR⁹R¹⁰. In an embodiment, R^{5c} is NR⁹R¹⁰ and R⁹ and R¹⁰ in R^{5c} are both CH₃ or both H. In an embodiment, one R^{5c} is NR⁹R¹⁰ and R⁹ and R¹⁰ in R^{5c} are both H.

In an embodiment, R⁹ and R¹⁰ in R^{5c} are each independently selected from H and C₁₋₄alkyl. In an embodiment, R⁹ and R¹⁰ in R^{5c} are each independently selected from H, CH₃, and CF₃ In an embodiment, one of R⁹ and R¹⁰ in R^{5c} is H and the other is CH₃. In an embodiment, R⁹ and R¹⁰ in R^{5c} are both CH₃.

In an embodiment, R⁹ and R¹⁰ in R^{5c} are joined to form, together with the nitrogen atom therebetween, a 3- to 7-membered saturated or unsaturated ring optionally containing one additional heteromoiety selected from N, NH, NC₁₋₆alkyl, O, S, S(O), and SO₂, and optionally substituted with one or more of halo and C₁₋₆alkyl. In an embodiment, R⁹ and R¹⁰ in R^{5c} are taken together with the nitrogen atom therebetween to form a 3- to 7-membered heterocyclic ring selected from azetidinyl, diazetidinyl, pyrrolidinyl, imidazolidinyl, pyrazolidinyl, thiazolidinyl, isothiozolidinyl, piperidinyl, diazinanyl (e.g. piperazinyl), morpholinyl and azepanyl, optionally substituted with one to four of halo and C₁₋₆alkyl. In an embodiment, R⁹ and R¹⁰ in R^{5c} are joined to form, together with the nitrogen atom therebetween, a 4 to 6-membered heterocyclic ring, optionally substituted with one to four of halo and C₁₋₆alkyl. In an embodiment R⁹ and R¹⁰ in R^{5c} are joined to form, together with the nitrogen atom therebetween, aziridinyl, azetidinyl, pyrrolidinyl, or piperidinyl, optionally substituted with one to three of F, Cl and C₁₋₄alkyl.

In an embodiment, Q is C₁₋₃alkylene optionally interrupted by NR⁴. In an embodiment, R⁴ is selected from H and C₁₋₄alkyl. Therefore, in an embodiment, wherein Q is C₁₋₃alkylene optionally interrupted by NR⁴, and R⁴ is selected from H and C₁₋₄alkyl. In an embodiment, R⁴ is selected from H, CH₃, CH₂CH₃, CF₂H, CF₃, CFH₂, CH₂CF₂H and CH₂CF₃.

In an embodiment, Q is C₁₋₃alkylene optionally interrupted by a heteromoiety selected from O, S, S(O), SO₂, and NR⁴. In an embodiment, Q is C₁₋₃alkylene optionally interrupted by a heteromoiety selected from O, SO₂, and NR⁴. In an embodiment, Q is C₁₋₃alkylene optionally interrupted by a heteromoiety selected from O and NR⁴. In an embodiment, Q is C₁₋₃alkylene optionally interrupted by a heteromoiety selected from O and SO₂. In an embodiment, Q is C₁₋₃alkylene optionally interrupted by O. In an embodiment, Q is C₁₋₃alkylene optionally interrupted by O. In an embodiment, Q is C₁₋₂alkylene optionally interrupted by SO₂.

In an embodiment, R⁴ is selected from H and C₁₋₄alkyl. In an embodiment, R⁴ is selected from H, CH₃, CH₂CH₃, CF₂H, CF₃, CFH₂, CH₂CF₂H, and CH₂CF₃.

In an embodiment, Q is C₁₋₃alkylene and optionally disubstituted on one carbon atom with R^{5a} and R^{5b} and R^{5a} and R^{5b} are joined to form, together with the nitrogen atom therebetween, a 3- to 6-membered saturated or unsaturated ring optionally containing one additional heteromoiety selected from N, NH, NC₁₋₆alkyl, O, S, S(O), and SO₂ and optionally substituted with one or more of halo and C₁₋₄alkyl. In an embodiment, Q is C₁alkylene or C₂alkylene, and is disubstituted on one carbon atom with R^{5a} and R^{5b} and R^{5a} and R^{5b} are joined to form, together with the carbon atom therebetween, a 3- to 5-membered cycloalkyl ring, and optionally substituted with one or more of halo and C₁₋₄alkyl. In an embodiment, Q is C₁alkylene or C₂alkylene, and disubstituted on one carbon atom with R^{5a} and R^{5b}. In an embodiment, Q is CR^{5a}R^{5b}. In an embodiment, R^{5a} and R^{5b} are joined to form, together with the carbon atom therebetween, a cyclopropyl or a cyclobutyl ring, and optionally substituted with one to four of halo and C₁₋₄alkyl. In an embodiment, Q is CR^{5a}R^{5b}. In an embodiment, R^{5a} and R^{5b} are joined to form, together with the carbon atom therebetween, a cyclopropyl or a cyclobutyl ring, and optionally substituted with one to three, one to two or one of halo and C₁₋₄alkyl.

In an embodiment, Q is C₂₋₄alkenylene optionally substituted with one or two of R^{5c}, and each R^{5c} is independently selected from F, C₁₋₄alkyl, C₃₋₆cycloalkyl, C₃₋₆heterocycloalkyl, C₁₋₄alkyleneC₃₋₆cycloalkyl, C₁₋₄alkyleneC₃₋₆heterocycloalkyl, OH, OC₁₋₆alkyl, NR⁹R¹⁰, and C₁₋₄alkyleneNR⁹R¹⁰, wherein all available hydrogen atoms are optionally substituted with a fluorine atom. In an embodiment, Q is C₂₋₄alkenylene optionally substituted with one or two of R^{5c}, and each R^{5c} is independently selected from F, Cl, C₁₋₄alkyl and OC₁₋₄alkyl. In an embodiment, Q is C═C optionally substituted with one or two of R^{5c}, and each R^{5c} is independently selected from F, Cl, C₁₋₄alkyl and OC₁₋₄alkyl. In an embodiment, Q is C═C optionally substituted with one or two of R^{5c}, and each R^{5c} is independently selected from F, Cl, CH₃ and OCH₃ OCF₃ and OCF₂H. In an embodiment, Q is C═C optionally substituted with one or two of R^{5c}, and R^{5c} is selected from F and C₁₋₄alkyl. In an embodiment, R^{5c} is selected from F and CH₃. In an embodiment, Q is C═C substituted with F. In an embodiment, Q is C═C substituted with Cl. In an embodiment, Q is C═C substituted with OCH₃. In an embodiment, Q is C═C substituted with CH₃. In an embodiment, Q is C═C substituted with F and CH₃.

In an embodiment, Q is selected from C═N and N═C and is optionally substituted with R^{5c}, and each R^{5c} is independently selected from F, C₁₋₄alkyl, C₃₋₆cycloalkyl, C₃₋₆heterocycloalkyl, C₁₋₄alkyleneC₃₋₆cycloalkyl, C₁₋₄alkyleneC₃₋₆heterocycloalkyl, OH, OC₁₋₆alkyl, NR⁹R¹⁰ and C₁₋₄alkyleneNR⁹R¹⁰, wherein all available hydrogen atoms are optionally substituted with a fluorine atom. In an embodiment, Q is selected from C═N and N═C and is optionally substituted with one or two of R^{5c}, and each R^{5c} is independently selected from F, Cl, C₁₋₄alkyl, OC₁₋₄alkyl, NR⁹R¹⁰, cyclopropyl and cyclobutyl. In an embodiment, Q is selected from C═N and N═C and is optionally substituted with one or two of R^{5c}, and each R^{5c} is independently selected from F, Cl, CH₃, OCH₃, OCF₃ OCF₂H, NR⁹R¹⁰, cyclopropyl and cyclobutyl. In an embodiment, Q is selected from C═N and N═C and is optionally substituted with one or two R^{5c}, and each R^{5c} is independently selected from F, Cl, CH₃, OCH₃, OCF₃ and OCF₂H. In an embodiment, Q is selected from C═N and N═C and is optionally substituted with R^{5c}, and R^{5c} is selected from F and CH₃. In an embodiment, Q is selected from C═N or N═C and is optionally substituted with R^{5c}, and R^{5c} is selected from cyclopropyl and cyclobutyl. In an embodiment, Q is selected from C═N or N═C and is optionally substituted with R^{5c}, and R^{5c} is selected from F and C₁₋₄alkyl. In an embodiment, Q is selected from C═N or N═C and is optionally substituted with one or two R^{5c}, and one R^{5c} is NR⁹R¹⁰. In an embodiment, Q is selected from C═N or N═C and is optionally substituted with one or two R^{5c}, and one R^{5c} is C₁₋₂alkyleneNR⁹R¹⁰.

In an embodiment, Cy¹ is C₆₋₁₆aryl or C₅₋₁₇heteroaryl, and Cy¹ is unsubstituted. In an embodiment, Cy¹ is C₆₋₁₆aryl or C₅₋₁₇heteroaryl, and Cy¹ is substituted with one to three of R¹¹. In an embodiment, Cy¹ is C₆₋₁₆aryl or C₅₋₁₇heteroaryl, and Cy¹ is substituted with Z-Cy². In an embodiment, Cy¹ is C₆₋₁₆aryl or C₅₋₁₇heteroaryl, and Cy¹ is substituted with Z-Cy² and one to three of R¹¹.

In an embodiment, Cy¹ is C₆₋₁₀aryl. Therefore, in an embodiment, Cy¹ is C₆₋₁₀aryl, and Cy¹ is unsubstituted, or is substituted with one or more of R⁹, or is substituted with Z-Cy², or is substituted with Z-Cy² and one or more of R¹¹. In an embodiment, Cy¹ is phenyl, indanyl or naphthyl. In an embodiment, Cy¹ is phenyl.

In an embodiment, Cy¹ is a bicyclic C₉₋₁₁aryl wherein the aryl group is fused to a heterocycloalkyl group, and Cy¹ is unsubstituted or is substituted with one or more of R¹¹ or is substituted with Z-Cy², or is substituted with Z-Cy² and one or more of R¹¹. In an embodiment, Cy¹ is a benzo-fused bicyclic C₉₋₁₁heterocycloalkyl. In an embodiment, Cy¹ is benzo-fused to a C₄₋₈heterocycloalkyl. In an embodiment, Cy¹ is selected from indolinyl, dihydrobenzofuranyl, dihydrobenzothiophenyl, benzofuranonyl, 1,1-dioxido-dihydrobenzothiophenyl, benzodioxolyl, benzodioxanyl, tetrahydroisoquinolinyl, tetrahydroquinolinyl, 1-oxo-tetrahydroisoquinolinyl, 1-oxo-tetrahydroquinolinyl, dihydrobenzodioxepinyl, benzoxazinyl, isochromanyl, chromanyl, chromanonyl, isochromenyl, chromenyl, and chromenonyl. In an embodiment, Cy¹ is selected from

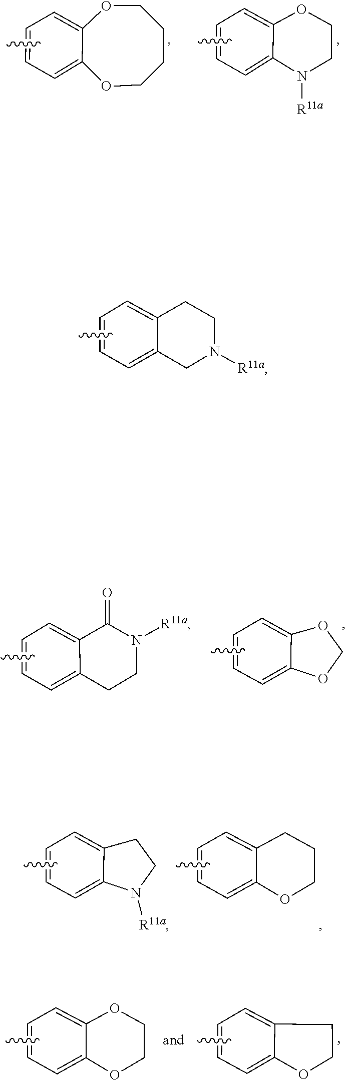

wherein [symbol] represents the point of attachment of Cy¹ to the remainder of molecule and the Cy¹ is unsubstituted or is substituted with one or more of R¹¹ or is substituted with Z-Cy², and wherein R^{11a} is selected from H, R¹¹ and Z-Cy².

In an embodiment, Cy¹ is a tricyclic C₁₁₋₁₇aryl wherein the aryl group is fused and/or spiro fused to one or two heterocycloalkyl groups, and Cy¹ is unsubstituted or is substituted with one or more of R¹¹ or is substituted with Z-Cy², or is substituted with Z-Cy² and one or more of R¹¹. In an embodiment, Cy¹ is a benzofused spirofused tricyclic C₁₁₋₁₇aryl. In an embodiment, Cy¹ is selected from spirobenzodioxinepiperidinyl, oxospirochromanepiperidinyl and spirochromanepiperidinyl. In an embodiment, Cy¹ is selected from

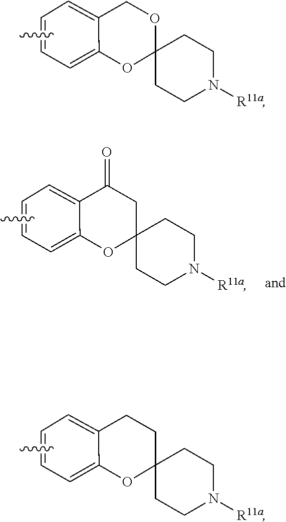

wherein [symbol] represents the point of attachment of Cy¹ to the remainder of molecule and the Cy¹ is unsubstituted or is substituted with one to three of R¹¹ or is substituted with Z-Cy², and wherein R^{11a} is selected from H, R¹¹ and Z-Cy².

In an embodiment, Cy¹ is C₅₋₆heteroaryl, and Cy¹ is unsubstituted, or is substituted with one or more of R¹¹, or is substituted with Z-Cy², or is substituted with one or more of Z-Cy² and R¹¹.

In an embodiment, Cy¹ is selected from pyrrolyl, imidazolyl, oxazolyl, pyrazolyl, thiazolyl, pyridinyl, pyrazinyl, pyridazinyl and pyrimidinyl. In an embodiment, Cy¹ is selected from pyrrolyl, pyrazolyl, thiazolyl, pyridinyl, pyrazinyl and pyrimidinyl. In an embodiment, Cy¹ is selected from pyrazolyl, pyridinyl, pyrazinyl and pyrimidinyl. In an embodiment, Cy¹ is pyridinyl. In an embodiment, Cy¹ is pyrazolyl.

In an embodiment, Cy¹ is bicyclic C₉₋₁₁heteroaryl, and Cy¹ is unsubstituted, or is substituted with one or more of R¹¹ or is substituted with Z-Cy², or is substituted with Z-Cy² and one or more of R¹¹. In an embodiment, Cy¹ is selected from benzofuranyl, benzothiophenyl, benzodioxolyl, quinolinyl, and isoquinolyl.

In an embodiment, Cy¹ is a pyrazolodiazepinonyl. In an embodiment, Cy¹ is

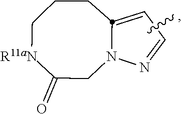

wherein [symbol] represents the point of attachment of Cy¹ to the remainder of molecule and the Cy¹ is unsubstituted or is substituted with one or more of R¹¹ or is substituted with Z-Cy², and wherein R^{11a} is selected from H, R¹¹ and Z-Cy².

In an embodiment, Cy¹ is unsubstituted or substituted with one to four of R¹¹. In an embodiment, Cy¹ is unsubstituted or substituted with one to three of R¹¹. In an embodiment, Cy¹ is unsubstituted or substituted with one to two of R¹¹. In an embodiment, Cy¹ is unsubstituted or substituted with one R¹¹. In an embodiment, Cy¹ is unsubstituted or substituted with Z-Cy² and one to four of R¹¹. In an embodiment, Cy¹ is unsubstituted or substituted with Z-Cy² and one to three of R¹¹. In an embodiment, Cy¹ is unsubstituted or substituted with Z-Cy² and one to two of R¹¹. In an embodiment, Cy¹ is unsubstituted or substituted with Z-Cy² and one of R¹¹.

In an embodiment, each R¹¹ is independently selected from halo, ═O, CN, NO₂, C₁₋₆alkyl, C₂₋₆alkenyl, C₂₋₆alkynyl, OR¹², C(O)R¹², CO₂R¹², P(O)R¹²R¹³, P(O)(OR¹²)(OR¹³), SR¹², S(O)R¹², SO₂R¹², S(O)(═NR¹³)R¹², SO₂NR¹²R¹³, SiR¹⁴R^{14a}R^{14a}, C₁₋₆alkyleneOR¹², OC₁₋₆alkyleneOR¹², C₁₋₆alkyleneNR^{12a}R^{13a}, OC₁₋₆alkyleneNR¹²R¹³, NR^{13a}C₁₋₆alkyleneNR¹²R¹³, NR^{13a}C₁₋₆alkyleneOR¹², NR^{12a}R^{13a}, NR^{13a}COR¹², NR^{13a}CO₂R¹², NR^{13a}SO₂R¹², C₃₋₇cycloalkyl, C₃₋₇heterocycloalkyl, C₁₋₄alkyleneC₃₋₇cycloalkyl and C₁₋₄alkyleneC₃₋₇heterocycloalkyl, the latter four groups being optionally substituted with one to three of R¹⁵.

In an embodiment, each R¹¹ is independently selected from F, Cl, ═O, CN, NO₂, C₁₋₄alkyl, OR¹², C(O)R¹², CO₂R¹², P(O)R¹²R¹³, P(O)(OR¹²)(OR¹³), SR¹², S(O)R¹², SO₂R¹², S(O)(═NR¹³)R¹², SO₂NR¹²R¹³, SiR¹⁴R^{14a}R^{14b}, C₁₋₄alkyleneOR¹², OC₁₋₆alkyleneOR¹², C₁₋₆alkyleneNR^{12a}R^{13a}, OC₁₋₄alkyleneNR¹²R¹³, NR^{13a}C₁₋₄alkyleneNR¹²R¹³, NR^{13a}C₁₋₄alkyleneOR¹², NR^{12a}R^{13a}, NR^{13a}COR¹², NR^{13a}CO₂R¹², and NR^{13a}SO₂R¹², and NR^{13a}SO₂R¹², C₃₋₇cycloalkyl, C₃₋₇heterocycloalkyl, C₁₋₄alkyleneC₃₋₇cycloalkyl and C₁₋₄alkyleneC₃₋₇heterocycloalkyl, the latter four groups being optionally substituted with one to three of R¹⁵.

In an embodiment, each R¹¹ is independently selected from F, Cl, CN, NO₂, CH₃, CH₂CH₃, CH(CH₃)₂, OR¹², C(O)R¹², CO₂R¹², P(O)R¹²R¹³, P(O)(OR¹²)(OR¹³), SR¹², SOR¹², SO₂R¹², S(O)(═NR¹³)R¹², SO₂NR¹²R¹³, SiR¹⁴R^{14a}R^{14b}, C₁₋₆alkyleneOR¹², OC₁₋₆alkyleneOR¹³, C₁₋₆alkyleneNR^{12a}R^{13a}, OC₁₋₆alkyleneNR¹²R¹³, NR^{13a}C₁₋₆alkyleneNR¹²R¹³, NR^{13a}C₁₋₆alkyleneOR¹², NR^{12a}R^{13a}, NR^{13a}COR¹², NR^{13a}CO₂R¹², NR^{13a}SO₂R¹², C₃₋₇cycloalkyl, C₃₋₇heterocycloalkyl, C₁₋₄alkyleneC₃₋₇cycloalkyl and C₁₋₄alkyleneC₃₋₇heterocycloalkyl, the latter four groups being optionally substituted with one to three of R¹⁵.

In an embodiment, each R¹¹ is independently selected from F, CH₃, CH₂CH₃, CH(CH₃)₂, P(O)R¹²R¹³, SO₂NR¹²R¹³, SiR¹⁴R^{14a}R^{14b}, OC₁₋₄alkyleneOR¹², C₁₋₄alkyleneNR^{12a}R^{13a}, NR^{12a}R^{13a}, NR^{13a}COR¹², NR^{13a}SO₂R¹², C₃₋₇cycloalkyl, C₃₋₇heterocycloalkyl, C₁₋₄alkyleneC₃₋₇cycloalkyl and C₁₋₄alkyleneC₃₋₇heterocycloalkyl, the latter four groups being optionally substituted with one to three of R¹⁵. In an embodiment, each R¹¹ is independently selected from F, CH₃, CH₂CH₃, CH(CH₃)₂, SO₂NR¹²R¹³, OC₁₋₆alkyleneOR¹², NR^{13a}C₁₋₆alkyleneNR¹²R¹³, C₃₋₇cycloalkyl, C₃₋₇heterocycloalkyl, C₁₋₄alkyleneC₃₋₇cycloalkyl and C₁₋₄alkyleneC₃₋₇heterocycloalkyl, the latter four groups being optionally substituted with one to three of R¹⁵. In an embodiment, one R¹¹ is SO₂NR¹²R¹³.

In an embodiment, each R¹¹ is independently selected from C₁₋₄alkyleneNR^{12a}R^{13a}, NR^{12a}R^{13a}, NR^{13a}COR¹², SO₂NR¹²R¹³, NR^{13a}C₁₋₄alkyleneNR¹²R¹³, C₃₋₇cycloalkyl, C₃₋₇heterocycloalkyl, C₁₋₄alkyleneC₃₋₇cycloalkyl and C₁₋₄alkyleneC₃₋₇heterocycloalkyl, the latter four groups being optionally substituted with one to three of R¹⁵. In an embodiment, each R¹¹ is independently selected from C₁₋₄alkyleneNR^{12a}R^{13a}, NR^{12a}R^{13a}NR^{13a}COR¹², SO₂NR¹²R¹³, NR^{13a}C₁₋₄alkyleneNR¹²R¹³, C₃₋₇heterocycloalkyl, and C₁₋₄alkyleneC₃₋₇heterocycloalkyl, the latter four groups being optionally substituted with one to four of R¹⁵. In an embodiment, one R¹¹ is SO₂NR¹²R¹³. In an embodiment, one or two R¹¹ are selected from C₁₋₄alkyleneNR^{12a}R^{13a}, NR^{12a}R^{13a}, NR^{13a}COR¹², NR^{13a}C₁₋₄alkyleneNR¹²R¹³, C₃₋₇heterocycloalkyl, and C₁₋₄alkyleneC₃₋₇heterocycloalkyl, the latter two groups being optionally substituted with one to four of R¹⁵. In an embodiment, one or two R¹¹ are selected from C₁₋₄alkyleneNR^{12a}R^{13a}, NR^{12a}R^{13a}, C₃₋₇heterocycloalkyl, and C₁₋₄alkyleneC₃₋₇heterocycloalkyl, the latter two groups being optionally substituted with one to four of R¹⁵. In an embodiment, one R¹¹ is selected from C₁₋₄alkyleneNR^{12a}R^{13a}, NR^{12a}R^{13a}, and NR^{13a}C₁₋₄alkyleneNR¹²R¹³. In an embodiment, one R¹¹ is NR¹³C₁₋₆alkyleneNR¹²R¹³. In an embodiment, one R¹¹ is C₁₋₄alkyleneNR^{12a}R^{13a}.

In an embodiment, one of R¹¹ is NR^{12a}R^{13a}, R^{12a} is selected from H and C₁₋₄alkyl wherein C₁₋₄alkyl is optionally substituted with one to three of R¹⁷ and R^{13a} is selected from H and C₁₋₄alkyl. In an embodiment, one of R¹¹ is NR^{12a}R^{13a}, R^{12a} is selected from H, CH₃, CH₂CH₃, CF₂H, CF₃, CFH₂, CH₂CF₂H, CH₂CF₃ wherein CH₃, CH₂CH₃, CF₂H, CF₃, CFH₂, CH₂CF₂H, CH₂CF₃ are optionally substituted with one to three of R¹⁷ and R^{13a} is selected from H, CH₃, CH₂CH₃, CF₂H, CF₃, CFH₂, CH₂CF₂H and CH₂CF. In an embodiment, one of R¹¹ is NR^{12a}R^{13a} and R^{12a} and R^{13a} are independently selected from H, CH₃, CH₂CH₃, CF₂H, CF₃, CFH₂, CH₂CF₂H and CH₂CF₃. In an embodiment, one of R¹¹ is NR^{12a}R^{13a} and R^{12a} and R^{13a} are independently selected from H, CF₃, CH₃ and CH₂CH₃. Accordingly, in an embodiment, R¹¹ is selected from NH₂, N(CH₃)₂, NH(CH₃), N(CH₃)(CH₂CH₂), NH(CH₂CH₂) and N(CH₂CH₂). In an embodiment, one of R¹¹ is selected from NH₂, N(CH₃)₂ and NH(CH₃).

In an embodiment, one of R¹¹ is C₁₋₄alkyleneNR^{12a}R^{13a} and R^{12a} is selected from H, C₁₋₄alkyl, C₂₋₄alkenyl, C₂₋₄alkynyl, C₁₋₄alkyleneC₃₋₁₀cycloalkyl, C₁₋₆alkyleneC₃₋₁₀heterocycloalkyl, C₁₋₆alkyleneOR¹⁶, and C₁₋₆alkyleneNR^{16a}R^{16b}, and all alkyl, alkenyl, alkynyl, alkylene and cycloalkyl groups in R^{12a} are optionally substituted with one to three of R¹⁷, and R^{13a} is selected from H and C₁₋₄alkyl. In an embodiment, one of R¹¹ is C₁₋₄alkyleneNR^{12a}R^{13a} and R^{12a} is selected from H, C₁₋₄alkyl, C₁₋₄alkyleneC₃₋₁₀cycloalkyl, C₁₋₄alkyleneC₃₋₁₀heterocycloalkyl, C₁₋₄alkyleneOR¹⁶, and C₁₋₄alkyleneNR^{16a}R^{16b} and each alkyl, alkylene and cycloalkyl groups of R^{12a} are optionally substituted with one to three of R¹⁷, and R^{13a} is selected from H and C₁₋₄alkyl

In an embodiment, one of R¹¹ is C₁₋₄alkyleneNR^{12a}R^{13a} and R^{12a} is selected from H and C₁₋₄alkyl wherein C₁₋₄alkyl is optionally substituted with one to three of R¹⁷ and R^{13a} is selected from H and C₁₋₄alkyl. In an embodiment, one of R¹¹ is C₁₋₄alkyleneNR^{12a}R^{13a}R^{12a} is selected from H, CH₃, CH₂CH₃, CF₂H, CF₃, CFH₂, CH₂CF₂H, CH₂CF₃ wherein CH₃, CH₂CH₃, CF₂H, CF₃, CFH₂, CH₂CF₂H, CH₂CF₃ are optionally substituted with one to three of R¹⁷ and R^{13a} is selected from H, CH₃, CH₂CH₃, CF₂H, CF₃, CFH₂, CH₂CF₂H and CH₂CF. In an embodiment, one of R¹¹ is C₁₋₄alkyleneNR^{12a}R^{13a} and R^{12a} and R^{13a} are independently selected from H, CH₃, CH₂CH₃, CF₂H, CF₃, CFH₂, CH₂CF₂H and CH₂CF₃. In an embodiment, one of R¹¹ is C₁₋₃alkylenNR^{12a}R^{13a} and R^{12a} and R^{13a} are independently selected from H, CF₃, CH₃ and CH₂CH₃. Accordingly, in an embodiment, R¹¹ is CH₂N(CH₂CH₃)₂, C(CH₃)₂NH₂, CH₂N(CH₃)₂, CH₂CH₂N(CH₃)₂ and CH₂N(CH₃)₂ and CH₂N(CH₃)₂. In an embodiment, R¹¹ is CH₂N(CH₃)₂. In an embodiment, one of R¹¹ is C₁₋₃alkyleneNR^{12a}R^{13a} and R^{12a} and R^{13a} are independently selected from H and CH₃. In an embodiment, one of R¹¹ is C₁₋₃alkyleneNR^{12a}R^{13a} and R^{12a} and R^{13a} are both H or are both CH₃.

In an embodiment, one of R¹¹ is C₁₋₄alkyleneNR^{12a}R^{13a} and R^{12a} is selected from C₁₋₆alkyleneOR¹⁶ and C₁₋₆alkyleneNR^{16a}R^{16b} and all alkylene groups in R^{12a} are optionally substituted with one to three of R¹⁷. In an embodiment, one of R¹¹ is C₁₋₄alkyleneNR^{12a}R^{13a} and R^{12a} is selected from C₁₋₄alkyleneOR¹⁶ and C₁₋₄alkyleneNR^{16a}R^{16b} and all alkylene groups in R^{12a} are optionally substituted with one to three of R¹⁷. In an embodiment, one of R¹¹ is C₁₋₄alkyleneNR^{12a}R^{13a}, R^{12a} is selected from C₁₋₄alkyleneOR¹⁶ and C₁₋₄alkyleneNR^{16a}R^{16b} and R^{13a} is selected from H and C₁₋₄alkyl and all alkylene groups in R^{12a} are optionally substituted with one to three of R¹⁷. Accordingly, in an embodiment, one of R¹¹ is C₁₋₄alkyleneN(R^{13a})(C₁₋₆alkyleneOR¹⁶) and R^{13a} is selected from H and C₁₋₄alkyl and all alkylene groups in R^{12a} are optionally substituted with one to three of R¹⁵.

In an embodiment, one of R¹¹ is C₁₋₄alkyleneNR^{12a}R^{13a}, R^{12a} is selected from C₁₋₄alkyleneOR¹⁶ and C₁₋₄alkyleneNR^{16a}R^{16b} and R^{13a} is selected from H and C₁₋₄alkyl. In an embodiment, one of R¹¹ is selected from C₁₋₄alkyleneNR^{12a}R^{13a}, R^{12a} is C₁₋₄alkyleneOR¹⁶ and C₁₋₄alkyleneNR^{16a}R^{16b} and R^{13a} is selected from H, CH₃, CH₂CH₃, CF₂H, CF₃, CFH₂, CH₂CF₂H and CH₂CF₃. In an embodiment, one of R¹¹ is C₁₋₄alkyleneNR^{12a}R^{13a}, R^{12a} is selected from C₁₋₄alkyleneOR¹⁶ and C₁₋₄alkyleneNR^{16a}R^{16b} and R^{13a} is selected from H, CH₃ and CH₂CH₃. In an embodiment, one of R¹¹ is C₁₋₄alkyleneNR^{12a}R^{13a}, R^{12a} is selected from C₁₋₄alkyleneOR¹⁶ and R^{13a} is selected from H and CH₃.

In an embodiment, one of R¹¹ is C₁₋₄alkyleneNR^{12a}R^{13a} and R^{12a} is selected from C₁₋₆alkyleneOR¹⁶ and R¹⁶ in R^{12a} is selected from H and C₁₋₄alkyl. In an embodiment, one of R¹¹ is C₁₋₄alkyleneNR^{12a}R^{13a} and R^{12a} is selected from C₁₋₆alkyleneOR¹⁶ and R¹⁶ in R^{12a} is selected from H, CH₃, CH₂CH₃, CF₂H, CF₃, CFH₂, CH₂CF₂H and CH₂CF₃. In an embodiment, one of R¹¹ is C₁₋₄alkyleneNR^{12a}R^{13a} and R^{12a} is selected from C₁₋₆alkyleneOR¹⁶ and R¹⁶ in R^{12a} is selected from H, CF₃ and CH₃. In an embodiment, one of R¹¹ is C₁₋₄alkyleneNR^{12a}R^{13a} and R^{12a} is selected from C₁₋₆alkyleneOR¹⁶ and R¹⁶ in R^{12a} is selected from H and CH₃.

Accordingly, in an embodiment, one of R¹¹ is selected from C₁₋₄alkyleneNH(C₁₋₆alkyleneOH), C₁₋₄alkyleneNCH₃(C₁₋₆alkyleneOH), C₁₋₄alkyleneNH(C₁₋₆alkyleneOCH₃), C₁₋₄alkyleneNCH₃(C₁₋₆alkyleneOCH₃). In an embodiment, one of R¹¹ is selected from C₁₋₄alkyleneNH(C₁₋₄alkyleneOH), C₁₋₄alkyleneNCH₃(C₁₋₆alkyleneOH), C₁₋₄alkyleneNH(C₁₋₄alkyleneOCH₃) and C₁₋₄alkyleneNCH₃(C₁₋₆alkyleneOCH₃). In an embodiment, one of R¹¹ is C₁₋₄alkyleneNR^{12a}R^{13a} and R^{12a} is C₁₋₆alkyleneC₃₋₇heterocycloalkyl and R^{13a} is selected from H and C₁₋₄alkyl. In an embodiment, one of R¹¹ is selected from C₁₋₄alkyleneC₃₋₇heterocycloalkyl and R^{12a} is C₁₋₄alkyleneC₃₋₇heterocycloalkyl and R^{13a} is selected from H and C₁₋₄alkyl. In an embodiment, one of R¹¹ is C₁₋₄alkyleneC₃₋₇heterocycloalkyl and R^{12a} is C₁₋₂alkyleneC₃₋₇heterocycloalkyl and R^{13a} is selected from H and C₁₋₄alkyl. In an embodiment, the C₃₋₇heterocycloalkyl in the C₁₋₄alkyleneC₃₋₇heterocycloalkyl of R^{12a} is selected from azetidinyl, oxetanyl, tetrahydrofuranyl, tetrahydrothiophenyl, pyrrolidinyl, pyrrolidin-2-onyl, imidazolidinyl, pyrazolidinyl, thiazolidinyl, isothiazolidinyl, dioxolanyl, dithiolanyl, 5,6-dihydro-1,2,4-triazinyl, 3,4,5,6-tetrahydro-1,2,4-triazinyl, thianyl, piperidinyl, piperazinyl, tetrahydropyranyl, thiomorpholinyl, morpholinyl, dioxanyl, azepanyl, diazepanyl, oxepanyl, thiepanyl, azabicyclohexanyl, azabicycloheptanyl, oxabicyclohexanyl, and oxabicycloheptanyl. In an embodiment, the C₃₋₇heterocycloalkyl in the C₁₋₄alkyleneC₃₋₇heterocycloalkyl of R^{12a} is selected from azetidinyl, tetrahydrofuranyl, pyrrolidinyl, pyrrolidin-2-onyl, piperidinyl, piperazinyl and morpholinyl.

In an embodiment, one or two of R¹¹ are independently selected from C₃₋₇cycloalkyl, C₃₋₇heterocycloalkyl, C₁₋₄alkyleneC₃₋₇cycloalkyl and C₁₋₄alkyleneC₃₋₇heterocycloalkyl, optionally substituted with one to three of R¹⁵. In an embodiment, one R¹ is selected from C₁₋₃alkyleneC₃₋₇cycloalkyl and C₃₋₇cycloalkyl, optionally substituted with one or two of R¹⁵. In an embodiment, the C₃₋₇cycloalkyl in the C₁₋₃alkyleneC₃₋₇cycloalkyl and C₃₋₇cycloalkyl of R¹¹ is selected from C₃₋₇cycloalkyl selected from cyclopropyl, cyclobutyl, cyclopentyl, cyclohexyl, cycloheptyl, bicyclo[2.2.1]hexanyl and bicyclo[2.2.1]heptanyl, optionally substituted with one or two of R¹⁵. In an embodiment, one R¹¹ is selected from C₃₋₇cycloalkyl, C₃₋₇heterocycloalkyl, C₁₋₄alkyleneC₃₋₇cycloalkyl and C₁₋₄alkyleneC₃₋₇heterocycloalkyl, optionally substituted with one to three of R¹⁵. In an embodiment, one R¹ is selected from C₁₋₃alkyleneC₃₋₇cycloalkyl and C₃₋₇cycloalkyl, optionally substituted with one or two of R¹⁵. In an embodiment, the C₃₋₇cycloalkyl in the C₁₋₃alkyleneC₃₋₇cycloalkyl and C₃₋₇cycloalkyl of R¹¹ is selected from C₃₋₇cycloalkyl selected from cyclopropyl, cyclobutyl, cyclopentyl, cyclohexyl, cycloheptyl, bicyclo[2.2.1]hexanyl and bicyclo[2.2.1]heptanyl, optionally substituted with one or two of R¹⁵.

In an embodiment, one R¹¹ is selected from C₃₋₇heterocycloalkyl and C₁₋₄alkyleneC₃₋₇heterocycloalkyl, optionally substituted with one to three of R¹⁵. In an embodiment, the C₃₋₇heterocycloalkyl in the C₃₋₇heterocycloalkyl and C₁₋₄alkyleneC₃₋₇heterocycloalkyl of R¹¹ is selected from azetidinyl, oxetanyl, tetrahydrofuranyl, tetrahydrothiophenyl, pyrrolidinyl, imidazolidinyl, pyrazolidinyl, thiazolidinyl, isothiazolidinyl, dioxolanyl, dithiolanyl, 5,6-dihydro-1,2,4-triazinyl, 3,4,5,6-tetrahydro-1,2,4-triazinyl, thianyl, piperidinyl, piperazinyl, tetrahydropyranyl, thiomorpholinyl, morpholinyl, dioxanyl, azepanyl, diazepanyl, oxepanyl, thiepanyl, azabicyclohexanyl, azabicycloheptanyl, oxabicyclohexanyl, and oxabicycloheptanyl optionally substituted with one to three of R¹⁵. In an embodiment, one R¹¹ is selected from C₄₋₆heterocycloalkyl and C₁₋₄alkyleneC₄₋₆heterocycloalkyl, and the C₄₋₆heterocycloalkyl in the C₄₋₆heterocycloalkyl and C₁₋₄alkyleneC₄₋₆heterocycloalkyl of R¹¹ is selected from azetidinyl, oxetanyl, tetrahydrofuranyl, tetrahydrothiophenyl, pyrrolidinyl, imidazolidinyl, pyrazolidinyl, thiazolidinyl, isothiazolidinyl, dioxolanyl, dithiolanyl, 5,6-dihydro-1,2,4-triazinyl, 3,4,5,6-tetrahydro-1,2,4-triazinyl, thianyl, piperidinyl, piperazinyl, tetrahydropyranyl, thiomorpholinyl, morpholinyl, and dioxanyl, optionally substituted with one to three of R¹⁵. In an embodiment, the C₄₋₆heterocycloalkyl in the C₄₋₆heterocycloalkyl and C₁₋₄alkyleneC₄₋₆heterocycloalkyl of R¹¹ is selected from tetrahydrofuranyl, tetrahydrothiophenyl, pyrrolidinyl, imidazolidinyl, pyrazolidinyl, thiazolidinyl and isothiazolidinyl, optionally substituted with one to three of R¹⁵. In an embodiment, the C₄₋₆heterocycloalkyl in the C₄₋₆heterocycloalkyl and C₁₋₄alkyleneC₄₋₆heterocycloalkyl of R¹¹ is selected from pyrrolidinyl, imidazolidinyl, pyrazolidinyl and thiazolidinyl, isothiazolidinyl, optionally substituted with one to three of R¹⁵. In some embodiments, the C₄₋₆heterocycloalkyl in the C₄₋₆heterocycloalkyl and C₁₋₄alkyleneC₄₋₆heterocycloalkyl of R¹¹ is selected from pyrrolidin-2-onyl, azabicyclohexanyl and azabicycloheptanyl. In some embodiments, the C₄₋₆heterocycloalkyl in the C₄₋₆heterocycloalkyl and C₁₋₄alkyleneC₄₋₆heterocycloalkyl of R¹¹ is pyrrolidin-2-onyl.

In an embodiment, the C₃₋₇heterocycloalkyl in the C₃₋₇heterocycloalkyl and C₁₋₄alkyleneC₃₋₇heterocycloalkyl of R¹¹ comprises at least one N atom. In an embodiment, the C₃₋₇heterocycloalkyl in the C₃₋₇heterocycloalkyl and C₁₋₄alkyleneC₃₋₇heterocycloalkyl of R¹¹ is selected from azetidinyl, pyrrolidinyl, pyrrolidin-2-onyl, azabicyclohexanyl, azabicycloheptanyl, piperidinyl, piperazinyl and morpholinyl each of which optionally substituted with one or two of R¹⁵. In an embodiment, the C₄₋₆heterocycloalkyl in the C₄₋₆heterocycloalkyl and C₁₋₄alkyleneC₄₋₆heterocycloalkyl of R¹¹ is pyrrolidinyl, optionally substituted with one or two of R¹⁵.

In an embodiment, one of R¹¹ is C₃₋₇heterocycloalkyl, optionally substituted with one to four of R¹⁵ and the C₃₋₇heterocycloalkyl is selected from azabicyclohexanyl, azabicycloheptanyl, pyrrolidinyl and pyrrolidin-2-onyl optionally substituted with one to four of R¹⁵. In an embodiment, one of R¹¹ is C₄₋₆heterocycloalkyl, optionally substituted with one to four of R¹⁵ and the C₄₋₆heterocycloalkyl is pyrrolidin-2-onyl. In an embodiment, one of R¹¹ is pyrrolidin-2-onyl selected from

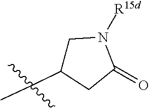

optionally substituted with one to three of R¹⁵, wherein R^{15d} is selected from H and R¹⁵, and

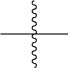

indicates a point of covalent attachment to Cy¹. In an embodiment, one R¹¹ is C₄₋₆heterocycloalkyl, optionally substituted with one or two of R¹⁵ and the C₄₋₆heterocycloalkyl is pyrrolidinyl. In an embodiment, one R¹¹ is pyrrolidine selected from

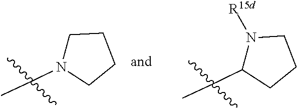

optionally substituted with one or two of R¹⁵, wherein R^{15d} is selected from H and R¹⁵, and

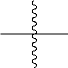

indicates a point of covalent attachment to Cy¹. In an embodiment, one R¹¹ C₄₋₆heterocycloalkyl, and the C₄₋₆heterocycloalkyl is pyrrolidinyl selected from

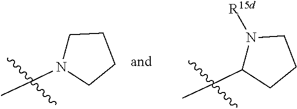

optionally substituted with one or two of R¹⁵, wherein R^{15d} is selected from H and R¹⁵, and

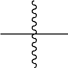

indicates a point of covalent attachment to Cy¹. In an embodiment, R^{15d} is H. In an embodiment, R^{15d} is R¹⁵.

In an embodiment, one of R¹¹ is C₁₋₆alkyleneC₃₋₇heterocycloalkyl, optionally substituted with one to four of R¹⁵, and the C₃₋₇heterocycloalkyl in the C₁₋₆alkyleneC₃₋₇heterocycloalkyl is selected from azetidinyl, pyrrolidinyl, pyrrolidin-2-onyl, piperidinyl, piperazinyl and morpholinyl. In an embodiment, one of R¹¹ is C₁₋₃alkyleneC₄₋₇ heterocycloalkyl, optionally substituted with one to four of R¹⁵, and the C₄₋₇heterocycloalkyl in the C₁₋₆alkyleneC₃₋₇heterocycloalkyl is selected from azetidinyl, pyrrolidinyl, piperidinyl, piperazinyl and morpholinyl. Accordingly, in an embodiment, one of R¹¹ is selected from C₁₋₃alkyleneazetidinyl, C₁₋₃alkylenepyrrolidinyl, C₁₋₃alkylenepiperidinyl, C₁₋₃alkylenepiperazinyl and C₁₋₃alkylenemorpholinyl optionally substituted with one to four of R¹⁵. In an embodiment, one of R¹¹ is selected from CH₂azetidinyl, CH₂pyrrolidinyl, CH₂piperidinyl, CH₂piperazinyl and CH₂morpholinyl optionally substituted with one to four of R¹⁵. In an embodiment, one of R¹¹ is selected from

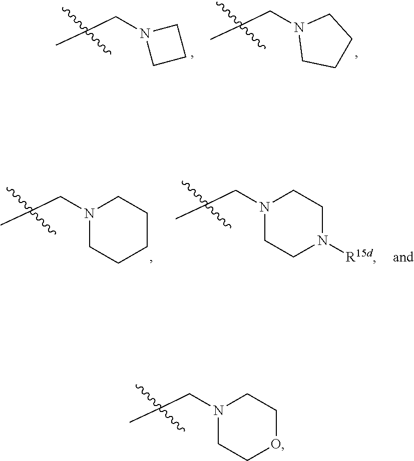

optionally substituted with one or two of R¹⁵, wherein R^{15d} is selected from H and R¹⁵, and

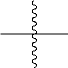

indicates a point of covalent attachment to Cy¹.

In an embodiment, one R¹¹ is C₁₋₆alkyleneC₃₋₇heterocycloalkyl, optionally substituted with one or two of R¹⁵, and the C₃₋₇heterocycloalkyl in the C₁₋₆alkyleneC₃₋₇heterocycloalkyl is pyrrolidinyl. In an embodiment, one R¹¹ is C₁₋₄alkylenepyrrolidinyl optionally substituted with one to three of R¹⁵. In an embodiment, one R¹¹ is C₁₋₄alkylenepyrrolidinyl optionally substituted with one to three of R¹⁵, wherein at least one R¹⁵ is F. In an embodiment, one of R¹¹ is selected from CH₂pyrrolidinyl optionally substituted with one to four of R¹⁵ In an embodiment, one of R¹¹ is

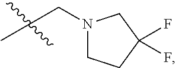 [SMILES: *CN1CCC(F)(F)C1]

wherein

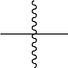

indicates a point of covalent attachment to Cy¹.

In an embodiment, each R¹⁵ is independently selected from F, Cl, CN, OH, C₁₋₄alkyl, OC₁₋₄alkyl and NR^{15a}R^{15b}. In an embodiment, each R¹⁵ is independently selected from F, Cl, OC₁₋₄alkyl, C₁₋₄alkyl and NR^{15a}R^{15b}.

In an embodiment, one R¹⁵ is selected from OH and OC₁₋₄alkyl and NR^{15a}R^{15b}. In an embodiment, one of R¹⁵ is selected from OH and OC₁₋₄alkyl. In an embodiment, one R¹⁵ is OC₁₋₄alkyl. In an embodiment, one of R¹⁵ is OCH₃.

In an embodiment, one of R¹¹ is C₁₋₆alkyleneC₃₋₇heterocycloalkyl as described above, substituted with one or two R¹⁵ and one R¹⁵ is selected from OH and OC₁₋₄alkyl. In an embodiment, one of R¹¹ is C₁₋₆alkyleneC₃₋₇heterocycloalkyl as described above, substituted with one R¹⁵ and R¹⁵ is OCH₃.

In an embodiment, each R¹⁵ is independently selected from F, C, CN, C₁₋₄alkyl and NR^{15a}R^{15b}. In an embodiment, each R¹⁵ is independently selected from F, C₁₋₄alkyl and NR^{15a}R^{15b}. In an embodiment, at least one R¹⁵ is F. In an embodiment, one R¹⁵ is NR^{15a}R^{15b}.

In an embodiment, R^{15a} and R^{15b} are independently selected from H and C₁₋₄alkyl. In an embodiment, R^{15a} and R^{15b} are independently selected from H, CH₃, CH₂CH₃, CF₂H, CF₃, CFH₂, CH₂CF₂H and CH₂CF₃. In an embodiment, R^{15a} and R^{15b} are independently selected from H, CF₃ and CH₃. In an embodiment, R^{15a} and R^{15b} are independently selected from H and CH₃.

In an embodiment, Cy¹ is substituted with one R¹¹ and R¹¹ is selected from C₃₋₇cycloalkyl, C₃₋₇heterocycloalkyl, C₁₋₄alkyleneC₃₋₇cycloalkyl and C₁₋₄alkyleneC₃₋₇heterocycloalkyl which are unsubstituted. In an embodiment, Cy¹ is substituted with one R¹¹ and R¹¹ is C₃₋₇heterocycloalkyl as defined above which is unsubstituted. In an embodiment, Cy¹ is substituted with one R¹¹ and R¹¹ is selected from pyrrolidinyl, imidazolidinyl, pyrazolidinyl, thiazolidinyl, and isothiazolidinyl, which is unsubstituted. In an embodiment, Cy¹ is substituted with one R¹¹ and R¹¹ is pyrrolidinyl which is unsubstituted. In an embodiment, Cy¹ is substituted with one R¹¹ and R¹¹ is pyrrolidin-2-only which is unsubstituted.

In an embodiment, Cy¹ is substituted with one R¹¹ and R¹¹ is selected from

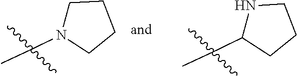 [SMILES: *N1CCCC1 ; *C1CCCN1]

wherein

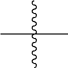

indicates a point of covalent attachment to Cy¹. In an embodiment, Cy¹ is substituted with one R¹¹ and R¹¹ is selected from

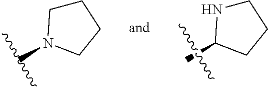

wherein

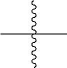

indicates a point of covalent attachment to Cy¹.

In an embodiment, one to three R¹¹ are independently selected from F, Cl, CN, NO₂ and C₁₋₄alkyl. In an embodiment, one to three R¹¹ are independently selected from F, Cl, CN, NO₂, CH₃, CH₂CH₃, CH₂CH₂CH₃, CH₂CH₂CH₂CH₃, CH(CH₃)₂, CH₂CH(CH₃)₂, C(CH₃) and CH(CH₃)₂. In an embodiment, one to three R¹¹ are independently selected from F, Cl, CN, CH₃, CH₂CH₃, CH(CH₃)₂, CH₂CH₂CH₃, CH₂CH₂CH₂CH₃, CF₂H, CF₃, CFH₂, CH₂CH₂F, CH₂CF₂H, CH₂CH₂F₂H, CH₂CH₂CH₂F₂H and CH(CH₃)₂. In an embodiment, one to three R¹¹ are independently selected from F, Cl, CN, CH₃, CH₂CH₃, CF₃, CF₂H, CH₂CF₂H, CH₂CF₃, CH₂CH₂F₂H, CH₂CH₂CH₂F₂H and CH(CH₃)₂ In an embodiment, one to three R¹¹ are independently selected from CH₃, CH₂CH₃, CF₃, CF₂H, CH₂CF₂H, CH₂CF₃, CH₂CH₂F₂H, CH₂CH₂CH₂F₂H and CH(CH₃)₂. In an embodiment, one or two R¹¹ are selected from CF₂H, CH₂CF₂H, CH₂CH₂F₂H, and CH₂CH₂CH₂F₂H. In an embodiment, one or two R¹¹ are selected from CH₂CH₂F₂H and CH₂CH₂CH₂F₂H. In an embodiment, one to three R¹¹ are independently selected from F, C, CN, CH₃, CH₂CH₃, CF₃, CH₂CF₂H, CH₂CF₃, and CH(CH₃)₂. In an embodiment, one to three R¹¹ are independently selected from F, CN, CH₃, CH₂CH₃, CF₃, CH₂CF₂H, CH₂CF₃, and CH(CH₃)₂. In an embodiment, one R¹¹ is CN. In an embodiment, one to three R¹¹ are independently selected from CF₃, CH₂CF₃, and F. In an embodiment, one to three R¹¹ are independently selected from CF₂H, CH₂CF₂H, CH₂CF₂H, CH₂CH₂F₂H and CH₂CH₂CH₂F₂H. In an embodiment, one to three of R¹¹ is F.

In an embodiment, each R¹¹ is independently selected from F, CH₃, CH₂CH₃, CH(CH₃)₂, OR¹², C(O)R¹², CO₂R¹², SR¹², SO₂R¹², S(O)(═NR¹³)R¹², C₁₋₄alkyleneOR¹², OC₁₋₄alkyleneOR¹², C₁₋₄alkyleneNR¹²R¹³, OC₁₋₄alkyleneOR¹², NR^{13a}C₁₋₄alkyleneOR¹², NR^{13a}COR¹², and NR^{13a}SO₂R¹², and R¹² is selected from H, CH₃, CH₂CH₃, CH(CH₃)₂, C(CH₃)₃, C₁₋₂alkyleneC₃₋₁₀cycloalkyl, C₁₋₂alkyleneC₃₋₁₀heterocycloalkyl, C₁₋₂alkylalkyleneOR¹⁶, and C₁₋₂alkylalkyleneNR^{16a}R^{16b}, and all alkyl, alkenyl, alkynyl, alkylene, heterocycloalkyl and cycloalkyl groups of R¹¹ are optionally substituted with one to three of R¹⁷. In an embodiment, one to three R¹¹ are independently selected from OR¹², C(O)R¹², CO₂R¹², SR¹², SO₂R¹², S(O)(═NR¹³)R¹², C₁₋₄alkyleneOR¹², OC₁₋₄alkyleneOR¹², NR^{13a}C₁₋₄alkyleneOR¹², NR^{13a}COR¹², and NR^{13a}SO₂R¹².

In an embodiment, R¹² and R^{12a} are independently selected from H, C₁₋₄alkyl, C₂₋₄alkenyl, C₂₋₄alkynyl, C₁₋₄alkyleneC₃₋₁₀cycloalkyl, C₁₋₆alkyleneC₃₋₁₀heterocycloalkyl, C₁₋₆alkyleneOR¹⁶ and C₁₋₆alkyleneNR^{16a}R^{16b}, all alkyl, alkenyl, alkynyl, alkylene, heterocycloalkyl and cycloalkyl groups of R¹² or R^{12a} are optionally substituted with one to three of R¹⁷. In an embodiment, R¹² and R^{12a} are independently selected from H, C₁₋₄alkyl, C₁₋₄alkyleneC₃₋₁₀cycloalkyl, C₁₋₄alkyleneC₃₋₁₀heterocycloalkyl, C₁₋₄alkyleneOR¹⁶, and C₁₋₄alkyleneNR^{16a}R^{16b} all alkyl, alkenyl, alkynyl, alkylene and cycloalkyl groups of R¹² or R^{12a} are optionally substituted with one to three of R¹⁷. In an embodiment, R¹² and R^{12a} are independently selected from H, CH₃, CH₂CH₃, CH(CH₃)₂, C(CH₃)₃, C₁₋₂alkyleneC₃₋₁₀cycloalkyl, C₁₋₂alkyleneC₃₋₁₀heterocycloalkyl, C₁₋₂alkyleneOR¹⁶, and C₁₋₂alkyleneNR^{16a}R^{16b} all alkyl, alkenyl, alkynyl, alkylene, heterocycloalkyl and cycloalkyl groups of R¹² or R^{12a} are optionally substituted with one to three of R¹⁷. In an embodiment, each R¹² is selected from H, CH₃, CH₂CH₃, CF₂H, CF₃, CFH₂, CH₂CF₂H, CH₂CF₃, CH₂CH₂F₂H, CH₂CH₂CH₂F₂H, CH(CH₃)₂, CH₂CH(CH₃)₂, C(CH₃), C₁₋₂alkyleneC₃₋₁₀cycloalkyl, C₁₋₂alkyleneC₃₋₁₀heterocycloalkyl, C₁₋₂alkyleneOR¹⁶, and C₁₋₂alkyleneNR^{16a}R^{16b} and CH₃, CH₂CH₃, CF₂H, CF₃, CFH₂, CH₂CF₂H, CH₂CF₃, CH₂CH₂F₂H, CH₂CH₂CH₂F₂H, CH(CH₃)₂, CH₂CH(CH₃)₂, C(CH₃), C₁₋₂alkyleneC₃₋₁₀cycloalkyl, C₁₋₂alkyleneC₃₋₁₀heterocycloalkyl, C₁₋₂alkyleneOR¹⁶, and C₁₋₂alkyleneNR^{16a}R^{16b} are optionally substituted with one to three of R¹⁷.

In an embodiment, R¹³ and R^{13a} are independently selected from H and C₁₋₄alkyl. In an embodiment, R¹³ and R^{13a} are independently selected from H, CH₃, CH₂CH₃, CF₂H, CF₃, CFH₂, CH₂CF₂H and CH₂CF₃. In an embodiment, R¹³ and R^{13a} are independently selected from H, CF₃ and CH₃. In an embodiment, R¹³ and R^{13a} is selected from H and CH₃. In an embodiment, R¹³ is independently selected from H and C₁₋₄alkyl. In an embodiment, each R¹³ is independently selected from H, CH₃, CH₂CH₃, CH(CH₃)₂, CH₂CH(CH₃)₂, C(CH₃)₃, CF₂H, CF₃, CFH₂, CH₂CF₂H and CH₂CF₃. In an embodiment, each R¹³ is independently selected from H, CF₃ and CH₃. In an embodiment, each R¹³ is independently selected from H and CH₃.

In an embodiment, R¹² and R¹³ are joined to form, together with the nitrogen atom therebetween, a 4- to 6-membered saturated or unsaturated ring, optionally containing one additional heteromoiety selected from N, NR¹⁸, O, S, S(O), and SO₂ and optionally substituted with one to three of R¹⁷. In an embodiment, R¹² and R¹³ are joined to form, together with the nitrogen atom therebetween, to form a 4- to 6-membered heterocycloalkyl ring selected from diazetidinyl, pyrrolidinyl, imidazolidinyl, pyrazolidinyl, thiazolidinyl, isothiozolidinyl, piperidinyl, diazinanyl (e.g. piperazinyl), and morpholinyl, and optionally substituted with one to three of R¹⁷. In an embodiment, R¹² and R¹³ are joined to form, together with the nitrogen atom therebetween, to form a 5- to 6-membered heterocycloalkyl ring selected from, pyrrolidinyl, imidazolidinyl, pyrazolidinyl, thiazolidinyl, isothiozolidinyl, piperidinyl, diazinanyl (e.g. piperazinyl) and morpholinyl, and optionally substituted with one to three of R¹⁷.

In an embodiment, one or two R¹¹ are independently selected from OR¹², C(O)R¹², CO₂R¹², SR¹², SO₂R¹², S(O)(═NR¹³)R¹², C₁₋₄alkyleneOR¹², OC₁₋₄alkyleneOR¹², NR^{13a}C₁₋₄alkyleneOR¹², NR^{13a}COR¹², and NR^{13a}SO₂R¹² and each R¹² is independently selected from H, C₁₋₄alkyl, C₁₋₄alkyleneC₃₋₁₀cycloalkyl, C₁₋₄alkyleneC₃₋₁₀heterocycloalkyl, C₁₋₄alkyleneOR¹⁶, and C₁₋₄alkyleneNR^{16a}R^{16b} and alkylene, heterocycloalkyl and cycloalkyl groups of R¹² are optionally substituted with one to three of R¹⁷, and R^{13a} is selected from H and C₁₋₄alkyl. In an embodiment, one or two R¹¹ are independently selected from OR¹², C(O)R¹², CO₂R¹², SR¹², SO₂R¹², S(O)(═NR¹³)R¹², C₁₋₄alkyleneOR¹², OC₁₋₄alkyleneOR¹², NR^{13a}C₁₋₄alkyleneOR¹², NR^{13a}COR¹², and NR^{13a}SO₂R¹² and R¹² is selected from H, CH₃, CH₂CH₃, CF₂H, CF₃, CFH₂, CH₂CF₂H, CH₂CF₃, CH₂CH₂F₂H, CH₂CH₂CH₂F₂H, CH(CH₃)₂, CH₂CH(CH₃)₂, C(CH₃)₃, C₁₋₂alkyleneC₃₋₁₀cycloalkyl, C₁₋₂alkyleneC₃₋₁₀heterocycloalkyl, C₁₋₂alkyleneOR¹⁶, and C₁₋₂alkyleneNR^{16a}R^{16b} and CH₃, CH₂CH₃, CF₂H, CF₃, CFH₂, CH₂CF₂H, CH₂CF₃, CH₂CH₂F₂H, CH₂CH₂CH₂F₂H, CH(CH₃)₂, CH₂CH(CH₃)₂, C(CH₃)₃, C₁₋₂alkyleneC₃₋₁₀cycloalkyl, C₁₋₂alkyleneC₃₋₁₀heterocycloalkyl, C₁₋₂alkyleneOR¹⁶, and C₁₋₂alkyleneNR^{16a}R^{16b} groups of R¹² are optionally substituted with one to three of R¹⁷, and R^{13a} is selected from H and C₁₋₄alkyl.

In an embodiment, one or two R¹¹ are independently selected from SR¹², SO₂R¹², S(O)(═NR¹³)R¹², NR^{13a}COR¹², and NR^{13a}SO₂R¹² and R¹² is selected from H, CH₃, CH₂CH₃, CF₂H, CF₃, CFH₂, CH₂CF₂H, CH₂CF₃, CH(CH₃)₂, CH₂CH(CH₃)₂, C(CH₃), C₁₋₂alkyleneC₃₋₁₀cycloalkyl, C₁₋₂alkyleneC₃₋₁₀heterocycloalkyl, C₁₋₂alkyleneOR¹⁶, and C₁₋₂alkyleneNR^{16a}R^{16b} and CH₃, CH₂CH₃, CF₂H, CF₃, CFH₂, CH₂CF₂H, CH₂CF₃, CH(CH₃)₂, CH₂CH(CH₃)₂, C(CH₃), C₁₋₂alkyleneC₃₋₁₀cycloalkyl, C₁₋₂alkyleneC₃₋₁₀heterocycloalkyl, C₁₋₂alkyleneOR¹⁶, and C₁₋₂alkyleneNR^{16a}R^{16b} groups of R¹² are optionally substituted with one to three of R¹⁷ and R^{13a} is selected from H and C₁₋₄alkyl.

In an embodiment, one or two R¹¹ are independently selected from SR¹² and SO₂R¹², R¹² is selected from H, CH₃, CH₂CH₃, CF₂H, CF₃, CFH₂, CH₂CF₂H, CH₂CF₃, CH₂CH₂F₂H, CH₂CH₂CH₂F₂H, CH(CH₃)₂, CH₂CH(CH₃)₂, C(CH₃), C₁₋₂alkyleneC₃₋₁₀cycloalkyl, C₁₋₂alkyleneC₃₋₁₀heterocycloalkyl, C₁₋₂alkyleneOR¹⁶, and C₁₋₂alkyleneNR^{16a}R^{16b} and all CH₃, CH₂CH₃, CF₂H, CF₃, CFH₂, CH₂CF₂H, CH₂CF₃, CH₂CH₂F₂H, CH₂CH₂CH₂F₂H, CH(CH₃)₂, CH₂CH(CH₃)₂, C(CH₃)₃, C₁₋₂alkyleneC₃₋₁₀cycloalkyl, C₁₋₂alkyleneC₃₋₁₀heterocycloalkyl, C₁₋₂alkyleneOR¹⁶ and C₁₋₂alkyleneNR^{16a}R^{16b} groups of R¹² are optionally substituted with one to three of R¹⁷. In an embodiment, one or two R¹¹ are independently selected from SR¹² and SO₂R¹², R¹² is selected from H, CH₃, CH₂CH₃, CF₂H, CF₃, CFH₂, CH₂CF₂H, CH₂CF₃, CH₂CH₂F₂H, CH₂CH₂CH₂F₂H, CH(CH₃)₂, CH₂CH(CH₃)₂ and C(CH₃)₃ wherein CH₃, CH₂CH₃, CF₂H, CF₃, CFH₂, CH₂CF₂H, CH₂CF₃, CH₂CH₂F₂H, CH₂CH₂CH₂F₂H, CH(CH₃)₂, CH₂CH(CH₃)₂, C(CH₃)₃ are optionally substituted with one to three of R¹⁷.

In an embodiment, one or two R¹¹ are independently selected from OR¹², C(O)R¹², CO₂R¹², C₁₋₄alkyleneOR¹², OC₁₋₄alkyleneOR¹², NR^{13a}C₁₋₄alkyleneOR¹² and R¹² is selected from H, CH₃, CH₂CH₃, CF₂H, CF₃, CFH₂, CH₂CF₂H, CH₂CF₃, CH₂CH₂F₂H, CH₂CH₂CH₂F₂H, CH(CH₃)₂, CH₂CH(CH₃)₂, C(CH₃)₃, C₁₋₂alkyleneC₃₋₁₀cycloalkyl, C₁₋₂alkyleneC₃₋₁₀heterocycloalkyl, C₁₋₂alkyleneOR¹⁶, and C₁₋₂alkyleneNR^{16a}R^{16b} and all CH₃, CH₂CH₃, CF₂H, CF₃, CFH₂, CH₂CF₂H, CH₂CF₃, CH₂CH₂F₂H, CH₂CH₂CH₂F₂H, CH(CH₃)₂, CH₂CH(CH₃)₂, C(CH₃)₃, C₁₋₂alkyleneC₃₋₁₀cycloalkyl, C₁₋₂alkyleneC₃₋₁₀heterocycloalkyl, C₁₋₂alkyleneOR¹⁶, and C₁₋₂alkyleneNR^{16a}R^{16b} groups of R¹² are optionally substituted with one to three of R¹⁷ and R^{13a} is selected from H and C₁₋₄alkyl.

In an embodiment, one or two R¹¹ are independently selected from OR¹², C(O)R¹², CO₂R¹², C₁₋₄alkyleneOR¹², OC₁₋₄alkyleneOR¹², OC₁₋₄alkyleneOR¹², NR^{13a}C₁₋₄alkyleneOR¹² and R¹² is selected from H, CH₃, CH₂CH₃, CF₂H, CF₃, CFH₂, CH₂CF₂H, CH₂CF₃, CH₂CH₂F₂H, CH₂CH₂CH₂F₂H, CH(CH₃)₂, CH₂CH(CH₃)₂, and C(CH₃)₃, wherein CH₃, CH₂CH₃, CF₂H, CF₃, CFH₂, CH₂CF₂H, CH₂CF₃, CH₂CH₂F₂H, CH₂CH₂CH₂F₂H, CH(CH₃)₂, CH₂CH(CH₃)₂, and C(CH₃)₃ in R¹² are optionally substituted with one to three of R¹⁷ and R^{13a} is selected from H and C₁₋₄alkyl. In an embodiment, one or two R¹¹ are independently selected from OR¹², C(O)R¹², CO₂R¹², C₁₋₄alkyleneOR¹² and OC₁₋₄alkyleneOR¹², and R¹² is selected from H, CH₃, CH₂CH₃, CF₂H, CF₃, CFH₂, CH₂CF₂H, CH₂CF₃, CH₂CH₂F₂H, CH₂CH₂CH₂F₂H, CH(CH₃)₂, CH₂CH(CH₃)₂, and C(CH₃)₃ wherein the CH₃, CH₂CH₃, CF₂H, CF₃, CFH₂, CH₂CF₂H, CH₂CF₃, CH₂CH₂F₂H, CH₂CH₂CH₂F₂H, CH(CH₃)₂, CH₂CH(CH₃)₂, and C(CH₃)₃ in R¹² are optionally substituted with one to three of R¹⁷. In an embodiment, one R¹¹ is selected from OR¹², C₁₋₄alkyleneOR¹², OC₁₋₄alkyleneOR¹², and R¹² is selected from H, CH₃, CH₂CH₃, CF₂H, CF₃, CFH₂, CH₂CF₂H, CH₂CF₃, CH₂CH₂F₂H, CH₂CH₂CH₂F₂H, CH(CH₃)₂, CH₂CH(CH₃)₂, and C(CH₃)₃ wherein the CH₃, CH₂CH₃, CF₂H, CF₃, CFH₂, CH₂CF₂H, CH₂CF₃, CH₂CH₂F₂H, CH₂CH₂CH₂F₂H, CH(CH₃)₂, CH₂CH(CH₃)₂, and C(CH₃)₃ in R¹² are optionally substituted with one to three of R¹⁷. In an embodiment, one R¹¹ is selected from OH, OCH₃, OCFH₂, OCH₂CF₂H, OCH₂CH₂F₂H, OCH₂CH₂CH₂F₂H, C₁₋₄alkyleneOH, C₁₋₄alkyleneOCH₃, OC₁₋₄alkyleneOH and OC₁₋₄alkyleneOCH₃. In an embodiment, one R¹¹ is selected from OH, OCH₃, OCFH₂, OCH₂CF₂H, OCH₂CH₂F₂H, OCH₂CH₂CH₂F₂H, CH₂OH, CH₂CH₂OH, CH₂CH₂OCH₃, CH₂OCH₃, OCH₂CH₂OH and OCH₂CH₂OCH₃.

In an embodiment, one R¹¹ is selected from OR¹², C₁₋₄alkyleneOR¹², OC₁₋₄alkyleneOR¹², and R¹² is selected from C₁₋₂alkyleneC₃₋₁₀cycloalkyl, C₁₋₂alkyleneC₃₋₁₀heterocycloalkyl, C₁₋₂alkyleneOR¹⁶, and C₁₋₂alkyleneNR^{16a}R^{16b} and alkylene, heterocycloalkyl and cycloalkyl groups in R¹² are optionally substituted with one to three of R¹⁷. In an embodiment, one R¹¹ is selected from OR¹², C₁₋₄alkyleneOR¹² and OC₁₋₄alkyleneOR¹², and R¹² is selected from C₁₋₂alkyleneC₃₋₆cycloalkyl, C₁₋₂alkyleneC₄₋₇heterocycloalkyl, and C₁₋₂alkyleneNR^{16a}R^{16b} and alkylene, heterocycloalkyl and cycloalkyl groups in R¹² are optionally substituted with one to three of R¹⁷. In an embodiment, the C₄₋₇cycloalkyl in R¹² is selected from cyclopropyl and cyclobutyl. Therefore, in an embodiment, one R¹¹ is selected from OR¹², C₁₋₄alkyleneOR¹² and OC₁₋₄alkyleneOR¹², and R¹² is selected from C₁₋₂alkylenecyclopropyl, C₁₋₂alkylenecyclobutyl, and C₁₋₂alkyleneC₄₋₇heterocycloalkyl. In an embodiment, one R¹¹ is selected from OR¹² and C₁₋₄alkyleneOR¹², and R¹² is selected from C₁₋₂alkylenecyclopropyl, C₁₋₂alkylenecyclobutyl, and C₁₋₂alkyleneC₄₋₇heterocycloalkyl. Accordingly, in an embodiment, one R¹¹ is selected from OC₁₋₂alkylenecyclopropyl, OC₁₋₂alkylenecyclobutyl, C₁₋₄alkyleneOC₁₋₂alkylenecyclobutyl, C₁₋₂alkyleneC₄₋₇heterocycloalkyl and OC₁₋₂alkyleneC₄₋₇heterocycloalkyl.

In an embodiment, one or two R¹¹ are independently selected from C(O)R¹² and CO₂R¹² and each R¹² is selected from H, CH₃, CH₂CH₃, CF₂H, CF₃, CFH₂, CH₂CF₂H, CH₂CF₃, CH(CH₃)₂, CH₂CH(CH₃)₂, and C(CH₃)₃ each of which is optionally substituted with one to three of R¹⁷. In an embodiment, one or two R¹¹ are independently selected from C(O)CH₃, C(O)C(CH₃)₃, CO₂CH₃ and CO₂C(CH₃)₃.

In an embodiment, each R¹⁷ is independently selected from F, Cl, CN, C₁₋₄alkyl and NR^{17a}R^{17b}. In an embodiment, each R¹⁷ is independently selected from F, C₁₋₄alkyl and NR^{17a}R^{17b}. In an embodiment, at least one R¹⁷ is F. In an embodiment, one R¹⁷ is NR^{17a}R^{17b}.

In an embodiment, R^{17a} and R^{17b} are independently selected from H and C₁₋₄alkyl. In an embodiment, R^{17a} and R^{17b} are independently selected from H, CH₃, CH₂CH₃, CF₂H, CF₃, CFH₂, CH₂CF₂H and CH₂CF₃. In an embodiment, R^{17a} and R^{17b} are independently selected from H, CF₃ and CH₃. In an embodiment, R^{17a} and R^{17b} are independently selected from H and CH₃.

In an embodiment, R¹⁴, R^{14a} and R^{14b} are independently selected from OR¹⁹, C₁₋₄alkyl, C₃₋₁₁cycloalkyl, C₃₋₁₀heterocycloalkyl, C₁₋₄alkyleneC₃₋₁₀cycloalkyl, and C₁₋₄alkyleneC₃₋₁₀heterocycloalkyl. In an embodiment, R¹⁴, R^{14a} and R^{14b} are independently selected from OR¹⁹, C₁₋₄alkyl, C₃₋₁₁cycloalkyl, C₃₋₁₀heterocycloalkyl, C₁₋₄alkyleneC₃₋₁₀cycloalkyl, and C₁₋₄alkyleneC₃₋₁₀heterocycloalkyl. In an embodiment, one of R¹⁴, R^{14a} and R^{14b} is OR¹⁹ and the other two of R¹⁴, R^{14a} and R^{14b} are selected from OR¹⁹, C₁₋₄alkyl, C₃₋₁₁cycloalkyl, C₃₋₁₀heterocycloalkyl, C₁₋₄alkyleneC₃₋₁₀cycloalkyl, and C₁₋₄alkyleneC₃₋₁₁heterocycloalkyl. In an embodiment, one of R¹⁴, R^{14a} and R^{14b} is OR¹⁹ and the other two of R¹⁴, R^{14a} and R^{14b} are independently selected from C₁₋₄alkyl, C₃₋₁₁cycloalkyl, C₃₋₁₀heterocycloalkyl, C₁₋₄alkyleneC₃₋₁₀cycloalkyl, and C₁₋₄alkyleneC₃₋₁₀heterocycloalkyl. In an embodiment, one of R¹⁴, R^{14a} and R^{14b} is OR¹⁹ and the other to of R¹⁴, R^{14a} and R^{14b} are selected from OR¹⁹, C₁₋₄alkyl, C₃₋₁₁cycloalkyl, C₃₋₁₀heterocycloalkyl, C₁₋₄alkyleneC₃₋₁₀cycloalkyl, and C₁₋₄alkyleneC₃₋₁₀heterocycloalkyl.

In an embodiment, R¹⁶, R^{16a}, R^{16b}, and R¹⁸ are independently selected from H and C₁₋₄alkyl, and alkyl groups are optionally fluoro-substituted. In an embodiment, R^{16a}, R^{16b}, and R¹⁸ are independently selected from H, CH₃, CH₂CH₃, CF₂H, CF₃, CFH₂, CH₂CF₂H and CH₂CF₃. In an embodiment, R¹⁶, R^{16a}, R^{16b}, and R¹⁸ are independently selected from H, CF₃ and CH₃. In an embodiment, R¹⁶, R^{16a}, R^{16b}, and R¹⁸ are independently selected from H and CH₃.

In an embodiment, R¹⁹ is selected from H, C₁₋₄alkyl, C₃₋₁₁cycloalkyl, C₃₋₁₀heterocycloalkyl, C₁₋₄alkyleneC₃₋₁₀cycloalkyl, C₁₋₄alkyleneC₃₋₁₀heterocycloalkyl C₁₋₄alkyleneOR²⁰, and C₁₋₄alkyleneNR²⁰R²¹. In an embodiment, R¹⁹ is selected from H and C₁₋₄alkyl. In an embodiment, R¹⁹ is H. In an embodiment R¹⁹ is CH₃.

In an embodiment, R²⁰ and R²¹ are independently selected from H and C₁₋₄alkyl.

In an embodiment, Cy¹ is phenyl, pyrrole or pyridinyl which is substituted with Z-Cy² and one or two of R¹¹. In an embodiment, Cy¹ is phenyl, pyrrole or pyridinyl which is substituted with Z-Cy² and one or two of R¹¹, and at least one R¹¹ is F. In an embodiment, Cy¹ is phenyl which is substituted with Z-Cy² and one or two of R¹¹, and at least one R¹¹ is F.

In an embodiment, Cy¹ is phenyl, pyrrole, or pyridinyl which is substituted with one or two of R¹¹, or with Z-Cy² and one or two of R¹¹. In an embodiment, Cy¹ is phenyl, pyrrole, or pyridinyl which is substituted with one or two of R¹¹, Z-Cy² and one or two of R¹¹, and at least one R¹¹ is F. In an embodiment, Cy¹ is phenyl which is substituted with one or two of R¹¹, or with Z-Cy² and one or two of R¹¹, and at least one R¹¹ is F.

In an embodiment, Cy¹ is phenyl, pyrrole or pyridinyl which is substituted with one or two of R¹¹, or is substituted with Z-Cy² and one to three R¹¹ and at least one R¹¹ is selected from OR¹², C(O)R¹², CO₂R¹², SR¹², SO₂R¹², S(O)(═NR¹³)R¹², NR^{13a}COR¹², and NR^{13a}SO₂R¹². In an embodiment, Cy¹ is phenyl which is substituted with one or two of R¹¹; or is substituted with Z-Cy² and one to three R¹¹ and at least one R¹¹ is selected from OR¹², C(O)R¹², CO₂R¹², SR¹², SO₂R¹², S(O)(═NR¹³)R¹², NR^{13a}COR¹², and NR^{13a}SO₂R¹². In an embodiment, R¹² is selected from H, CH₃, CH₂CH₃, CH(CH₃)₂, C(CH₃)₃, C₁₋₂alkyleneC₃₋₁₀cycloalkyl, C₁₋₂alkyleneC₃₋₁₀heterocycloalkyl, C₁₋₂alkyleneOR¹⁶, and C₁₋₂alkyleneNR¹³R¹⁶. In an embodiment, R¹² is selected from C₁₋₂alkyleneC₃₋₁₀cycloalkyl, and C₁₋₂alkyleneC₃₋₁₀heterocycloalkyl.

In an embodiment, Cy¹ is phenyl, pyrrole or pyridinyl which is substituted with one to three of R¹¹, or is substituted with Z-Cy² and one to three R¹¹ and at least one R¹¹ is selected from SO₂NR¹²R¹³ and NR^{13a}C₁₋₆alkyleneNR¹²R¹³. In an embodiment, Cy¹ is phenyl which is substituted with one to three of R¹¹, or is substituted with Z-Cy² and one to three R¹¹ and one or two of R¹¹ is selected from SO₂NR¹²R¹³ and NR^{13a}C₁₋₆alkyleneNR¹²R¹³. In an embodiment, Cy¹ is phenyl which is substituted with one to three of R¹¹, or is substituted with Z-Cy² and one to three R¹¹ and one of R¹¹ is SO₂NR¹²R¹³. In an embodiment, Cy¹ is phenyl which is substituted with one to three of R¹¹, or is substituted with Z-Cy² and one to three R¹¹ and one R¹¹ is NR^{13a}C₁₋₆alkyleneNR¹²R¹³. In an embodiment each R¹², R¹³ and R^{13a} are independently selected from H and C₁₋₄alkyl.

In an embodiment, Cy¹ is phenyl, pyrrole or pyridinyl which is substituted with one to three of R¹¹, or is substituted with Z-Cy² and one to three R¹¹ and at least one R¹¹ is SiR¹⁴R^{14a}R^{14b}. In an embodiment, Cy¹ is phenyl which is substituted with one to three of R¹¹, or is substituted with Z-Cy² and one to three R¹¹ and one or two of R¹¹ is SiR¹⁴R^{14a}R^{14b}. In an embodiment, one of R¹⁴, R^{14a} and R^{14b} is OR¹⁹ and the others are selected from H, OR¹⁹, C₁₋₄alkyl, C₃₋₁₁cycloalkyl, C₃₋₁₀heterocycloalkyl, C₁₋₄alkyleneC₃₋₁₀cycloalkyl, and C₁₋₄alkyleneC₃₋₁₀heterocycloalkyl.

In an embodiment, Cy¹ is phenyl or pyridinyl which is substituted with one to three of R¹¹, or is substituted with Z-Cy² and one to three R¹¹ and one or two R¹¹ are selected from C₁₋₄alkyleneNR^{12a}R^{13a}, NR^{12a}R^{13a}, SO₂NR¹²R¹³, NR^{13a}COR¹², NR^{13a}C₁₋₄alkyleneNR¹²R¹³, OR¹², OC₁₋₄alkyleneOR¹², C₃₋₇heterocycloalkyl, and C₁₋₄alkyleneC₃₋₇heterocycloalkyl, the latter two groups being optionally substituted with one to four of R¹⁵. In an embodiment, Cy¹ is phenyl or pyridinyl which is substituted with one to three of R¹¹, or is substituted with Z-Cy² and one to three R¹¹ and one or two R¹¹ are selected from C₁₋₄alkyleneNR^{12a}R^{13a}, NR^{12a}R^{13aa}, NR^{13a}C₁₋₄alkyleneNR¹²R¹³, C₃₋₇heterocycloalkyl, and C₁₋₄alkyleneC₃₋₇heterocycloalkyl, the latter two groups being optionally substituted with one to four of R¹⁵. In an embodiment, Cy¹ is phenyl or pyridinyl which is substituted with one to three of R⁹, or is substituted with Z-Cy² and one to three R¹¹ and one or two R¹¹ are selected from C₁₋₄alkyleneNR^{12a}R^{13a}. In an embodiment, Cy¹ is phenyl or pyridinyl which is substituted with one to three of R¹¹, or is substituted with Z-Cy² and one to three R¹¹ and one or two R¹¹ are selected from C₁₋₄alkylene NR^{12a}R^{13a}, NR^{12a}R^{13a}, OR¹², C₁₋₄alkyleneOR¹², C₃₋₇heterocycloalkyl, and C₁₋₄alkyleneC₃₋₇heterocycloalkyl, the latter two groups being optionally substituted with one to four of R¹⁵. In an embodiment, Cy¹ is phenyl or pyridinyl which is substituted with one to three of R¹¹, or is substituted with Z-Cy² and one to three R¹¹ and one or two R¹¹ are selected from C₁₋₄alkylene NR^{12a}R^{13a} and OR¹². In an embodiment, Cy¹ is phenyl or pyridinyl which is substituted with one to three of R¹¹, or is substituted with Z-Cy² and one to three R¹¹ and one or two R¹¹ are selected from CH₂N(CH₂CH₃)₂, C(CH₃)₂NH₂, CH₂N(CH₃)₂, CH₂CH₂N(CH₃)₂ and CH₂N(CH₃)₂ and CH₂N(CH₃)₂. In an embodiment, Cy¹ is phenyl or pyridinyl which is substituted with one to three of R¹¹, or is substituted with Z-Cy² and one to three R¹¹ and one or two R¹¹ are selected from C₁₋₄alkyleneC₃₋₇heterocycloalkyl optionally substituted with one to four of R¹⁵.

In an embodiment, Cy¹ is phenyl or pyridinyl which is substituted with one to three of R¹¹, or is substituted with Z-Cy² and one of R¹¹ is C₁₋₄alkyleneNR^{12a}R^{13a}, R^{12a} is selected from C₁₋₄alkyleneOR¹⁶ and C₁₋₄alkyleneNR^{16a}R^{16b} and R^{13a} is selected from H and C₁₋₄alkyl and all alkylene groups in R^{12a} are optionally substituted with one to three of R¹⁷. In an embodiment, Cy¹ is phenyl or pyridinyl which is substituted with one to three R¹¹ or is substituted with Z-Cy² and one to three R¹¹, and one R¹¹ is C₁₋₄alkyleneN(C₁₋₆alkyleneOR¹)(R^{13a}). In an embodiment, Cy¹ is phenyl or pyridinyl which is substituted with one to three of R¹¹, or is substituted with Z-Cy² and one to three R¹¹ and one or two R¹¹ are selected from C₁₋₄alkyleneNR^{12a}R^{13a} and R^{12a} is selected from C₃₋₇heterocycloalkyl and C₁₋₄alkyleneC₃₋₇heterocycloalkyl and R^{13a} is selected from H and C₁₋₄alkyl. In an embodiment, Cy¹ is phenyl or pyridinyl which is substituted with one to three of R¹¹, or is substituted with Z-Cy² and one to three R¹¹ and one or two R¹¹ are selected from C₁₋₄alkyleneNR^{12a}R^{13a} and R^{12a} is selected from C₃₋₇heterocycloalkyl and C₁₋₄alkyleneC₃₋₇heterocycloalkyl and R^{12a} is selected from H and C₁₋₄alkyl wherein the C₃₋₇heterocycloalkyl in the C₁₋₄alkyleneC₃₋₇heterocycloalkyl is selected from azetidinyl, pyrrolidinyl, pyrrolidin-2-onyl, piperidinyl, piperazinyl and morpholinyl.

In an embodiment, Cy¹ is phenyl, pyrrole, or pyridinyl which is substituted with one to three of R¹¹, and one R¹¹ is pyrrolidinyl optionally substituted with one or more of R¹⁵. In an embodiment, Cy¹ is phenyl which is substituted with one to three of R¹¹ and one R¹¹ is pyrrolidinyl, optionally substituted with one to four of R¹⁵.

In an embodiment, Cy¹ is phenyl, pyrrole, or pyridinyl which is substituted with one to three of R¹¹, or is substituted with Z-Cy² and one to three R¹¹ and one R¹¹ is C₁₋₄alkylenepyrrolidinyl optionally substituted with one to four of R¹⁵. In an embodiment, Cy¹ is phenyl which is substituted with one to three of R¹¹, or is substituted with Z-Cy² and one to three R¹¹ and one R¹¹ is C₁₋₄alkylenepyrrolidinyl optionally substituted with one or two of R¹⁵.

In an embodiment, Cy¹ is a bicyclic C₉₋₁₁aryl or tricyclic C₁₁₋₁₆aryl wherein the aryl group is fused and/or spiro fused, to one or two heterocycloalkyl groups, and Cy¹ is unsubstituted. In an embodiment, Cy¹ is a bicyclic C₉₋₁₁aryl or tricyclic C₁₁₋₁₆aryl wherein the aryl group is fused and/or spiro fused, to one or two heterocycloalkyl groups, and Cy¹ is substituted with one to three of R¹¹, or is substituted with Z-Cy² and one to three R¹¹ and each R¹¹ is independently selected from F, Cl, CN, NO₂, C₁₋₄alkyl, OR¹², C(O)R¹², CO₂R¹², SR¹², SO₂R¹², C₁₋₆alkyleneOR¹². In an embodiment, Cy¹ is a bicyclic C₉₋₁₁aryl wherein the aryl is fused to a heterocycloalkyl, and Cy¹ is unsubstituted, or is substituted with one or more of R, or is substituted with Z-Cy², or is substituted with Z-Cy² and one or more of R¹¹. In an embodiment, Cy¹ is a bicyclic C₉₋₁₁aryl wherein the aryl group is fused to a heterocycloalkyl group, and Cy¹ is substituted with one to three of R¹¹, or is substituted with Z-Cy² and one to three R¹¹ and each R¹¹ is independently selected from F, Cl, CN, NO₂, C₁₋₄alkyl, OR¹², and C₁₋₆alkyleneOR¹². In an embodiment, Cy¹ is a tricyclic C₁₁₋₁₆aryl, wherein the aryl group is fused and/or spirofused to two heterocycloalkyl groups, and Cy¹ is substituted with one to three of R¹¹; or is substituted with Z-Cy² and one to three R¹¹ and each R¹¹ is independently selected from F, C₁₋₄alkyl, OR¹², C(O)R¹², CO₂R¹², and C₁₋₆alkyleneOR¹². In an embodiment, Cy¹ is a tricyclic C₁₁₋₁₆aryl, wherein the aryl group is fused and/or spirofused to two heterocycloalkyl groups, and Cy¹ is substituted with one to two of R¹¹, or is substituted with Z-Cy² and one to three R¹¹ and at least one R¹¹ is selected from OR¹², C(O)R¹², and CO₂R¹². In an embodiment, R¹² is selected from H, CH₃, CH₂CH₃, CF₂H, CF₃, CFH₂, CH₂CF₂H, CH₂CF₃, and C(CH₃)₃. In some embodiments, each R¹¹ is independently selected from C₁₋₄alkyleneNR^{12a}R^{13a} and NR^{12a}R^{13a}. In some embodiments, the aryl is phenyl, therefore in an embodiment, Cy¹ is a benzofused bicyclic or tricyclic aryl group where phenyl is fused and/or spirofused to one or two heterocycloalkyl groups.

In an embodiment, Cy¹ is a benzo-fused bicyclic C₉₋₁₁aryl wherein the aryl group is a phenyl and is fused to a heterocycloalkyl group, or tricyclic C₁₁₋₁₆aryl wherein the aryl group is phenyl and is fused and/or spiro fused to one or two heterocycloalkyl groups, all of which are substituted with one to three of R¹¹, or is substituted with Z-Cy² and one to three R¹¹ and R¹¹ is selected from CH₃, CF₂H, CFH₂, CH₂CF₂H, CH₂CH₂F₂H, CH₂CH₂CH₂F₂H, OCH₃, OCF₂H, OCFH₂, OCH₂CF₂H, OCH₂CH₂F₂H, OCH₂CH₂CH₂F₂H, C₁₋₄alkyleneNR^{12a}R^{13a} and NR^{12a}R^{13a}.

In an embodiment, Z is absent. It is appreciated by a person skilled in the art that when Z is absent, Cy² is covalently attached to Cy¹ by a direct bond.

In an embodiment, Z is selected from C₁₋₄alkylene, O, C(O), CO₂, SO₂, S(O)(═NHR^{13b}) and NR^{13b}. In an embodiment, Z is selected from C₁₋₄alkylene, O, C(O), and SO₂. In an embodiment, Z is O. In an embodiment, Z is SO₂.

In an embodiment, Z is selected from C₁₋₆alkyleneO, C₁₋₆alkyleneC(O), C₁₋₆alkyleneCO₂, C₁₋₆alkyleneS, C₁₋₆alkyleneS(O), C₁₋₆alkyleneSO₂, C₁₋₆alkyleneS(O)(═NR^{13b}), C₁₋₆alkyleneNR^{13b}, OC₁₋₆alkylene, C(O)C₁₋₆alkylene, CO₂C₁₋₆alkylene, SC₁₋₆alkylene, S(O)C₁₋₆alkylene, SO₂C₁₋₆alkylene, S(O)(═NR^{13b})C₁₋₆alkylene and NR^{13b}C₁₋₆alkylene. In an embodiment, Z is selected from C₁₋₄alkyleneO, C₁₋₄alkyleneC(O), C₁₋₆alkyleneCO₂, C₁₋₄alkyleneS, C₁₋₄alkyleneS(O), C₁₋₄alkyleneSO₂, C₁₋₄alkyleneS(O)(═NR^{13b}), C₁₋₄alkyleneNR^{13b}, OC₁₋₄alkylene, C(O)C₁₋₄alkylene, CO₂C₁₋₄alkylene, SC₁₋₄alkylene, S(O)C₁₋₄alkylene, SO₂C₁₋₄alkylene, S(O)(═NR^{13b})C₁₋₄alkylene and NR^{13b}C₁₋₆alkylene. In an embodiment, Z is selected from C₁₋₄alkyleneO, C₁₋₄alkyleneC(O), C₁₋₆alkyleneCO₂, C₁₋₄alkyleneS, C₁₋₄alkyleneS(O), C₁₋₄alkyleneSO₂, C₁₋₄alkyleneNR^{13b}, OC₁₋₄alkylene, C(O)C₁₋₄alkylene, CO₂C₁₋₄alkylene, SC₁₋₄alkylene, S(O)C₁₋₄alkylene, SO₂C₁₋₄alkylene, and NR^{13b}C₁₋₆alkylene. In an embodiment, Z is selected from C₁₋₄alkyleneO, C₁₋₄alkyleneC(O), OC₁₋₄alkylene and C(O)C₁₋₄alkylene. In an embodiment, Z is selected from C₁₋₄alkyleneO, C₁₋₄alkyleneC(O), C₁₋₆alkyleneCO₂, C₁₋₄alkyleneS, C₁₋₄alkyleneS(O), C₁₋₄alkyleneSO₂, C₁₋₄alkyleneNR^{13b}, OC₁₋₄alkylene, C(O)C₁₋₄alkylene, CO₂C₁₋₄alkylene, SC₁₋₄alkylene, S(O)C₁₋₄alkylene, SO₂C₁₋₄alkylene, and NR^{11b}C₁₋₆alkylene. In an embodiment, Z is selected from OC₁₋₄alkylene and C(O)C₁₋₄alkylene. In an embodiment, Z is OC₁₋₄alkylene.

In an embodiment, R^{13b} is selected from H and C₁₋₄alkyl. In an embodiment, R^{13b} is selected from H, CH₃, CH₂CH₃, CF₂H, CF₃, CFH₂, CH₂CF₂H and CH₂CF₃. In an embodiment, R^{13b} is selected from CH₂CH₂CF₂H and CH₂CH₂CH₂CF₂H. In an embodiment, R^{13b} is selected from H, CF₃ and CH₃. In an embodiment, R^{13b} is selected from H and CH₃.

In an embodiment, Cy² is C₃₋₁₁cycloalkyl and Cy² is unsubstituted or substituted with one or more of R²². In an embodiment, Cy² is selected from cyclopropyl, cyclobutyl, cyclopentyl, cyclohexyl, and cycloheptyl. In an embodiment, Cy² is cyclopropyl.

In an embodiment, Cy² is a monocyclic C₃₋₇heterocycloalkyl and Cy² is unsubstituted or substituted with one or more of R²². In an embodiment, Cy² is selected from azetidinyl, oxetanyl, tetrahydrofuranyl, tetrahydrothiophenyl, pyrrolidinyl, dihydropyrrolyl, imidazolidinyl, pyrazolidinyl, thiazolidinyl, isothiazolidinyl, dioxolanyl, dithiolanyl, 5,6-dihydro-1,2,4-triazinyl, 3,4,5,6-tetrahydro-1,2,4-triazinyl, dioxidothiomorpholino, tetrahydropyridinyl, dihydropyridinyl, dihydropyranyl, thianyl, piperidinyl, piperazinyl, tetrahydrofuranyl, tetrahydropyranyl, thiomorpholinyl, morpholinyl, dioxanyl, azepanyl, diazepanyl, oxepanyl and thiepanyl and Cy² is unsubstituted or substituted with one or more of R²². In an embodiment, Cy² is selected from azetidinyl, 5,6-dihydro-1,2,4-triazinyl, dioxidothiomorpholino, thiomorpholinyl, tetrahydrofuranyl, tetrahydropyranyl, diazepanyl, pyrrolidinyl, piperidinyl, piperazinyl and morpholinyl and Cy² is unsubstituted or substituted with one to three of R²². In an embodiment, Cy² is selected from tetrahydrofuranyl, tetrahydropyranyl, pyrrolidinyl, piperidinyl, piperazinyl and morpholinyl, and Cy² is unsubstituted or substituted with one to three of R²². In an embodiment, Cy² is selected from pyrrolidinyl, piperidinyl, piperazinyl and morpholinyl and Cy² is unsubstituted or substituted with one to three of R²². In an embodiment, Cy² is dihydropyranyl. In an embodiment, Cy² is selected from piperidinyl, piperazinyl, and morpholinyl, and Cy² is unsubstituted or substituted with one or two of R²². In an embodiment, Cy² is piperazinyl and Cy² is unsubstituted or substituted with one or two of R²². In an embodiment, Cy² is selected from tetrahydrofuranyl, dihydropyranyl, morpholinyl, and tetrahydropyranyl, which is substituted with one to three of R²². In an embodiment, Cy² is tetrahydropyranyl, which is substituted with one to three of R²².

In an embodiment, Cy² is selected from

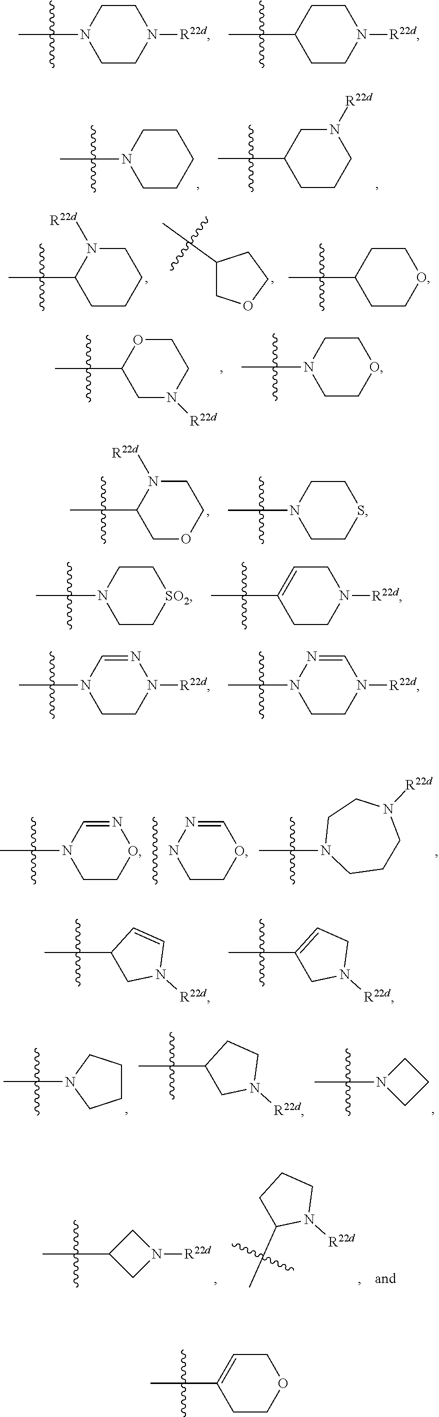

which is unsubstituted or substituted with one to four of R²², and
wherein
R^{22d} is selected from H and R²²; and

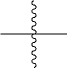

indicates a point of covalent attachment to Z.

In an embodiment, Cy² is selected from

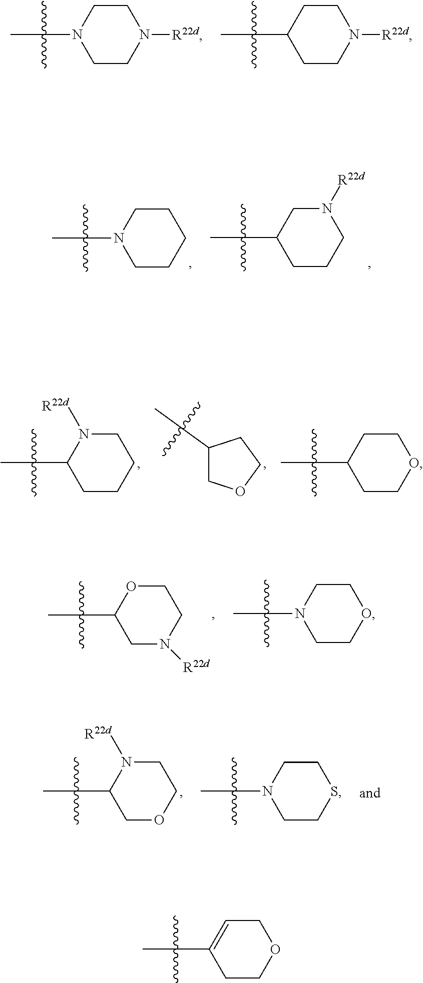

which is unsubstituted or substituted with one to four of R²², and
wherein
R^{22d} is selected from H and R²²; and

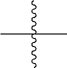

indicates a point of covalent attachment to Z.

In an embodiment, Cy² is selected from

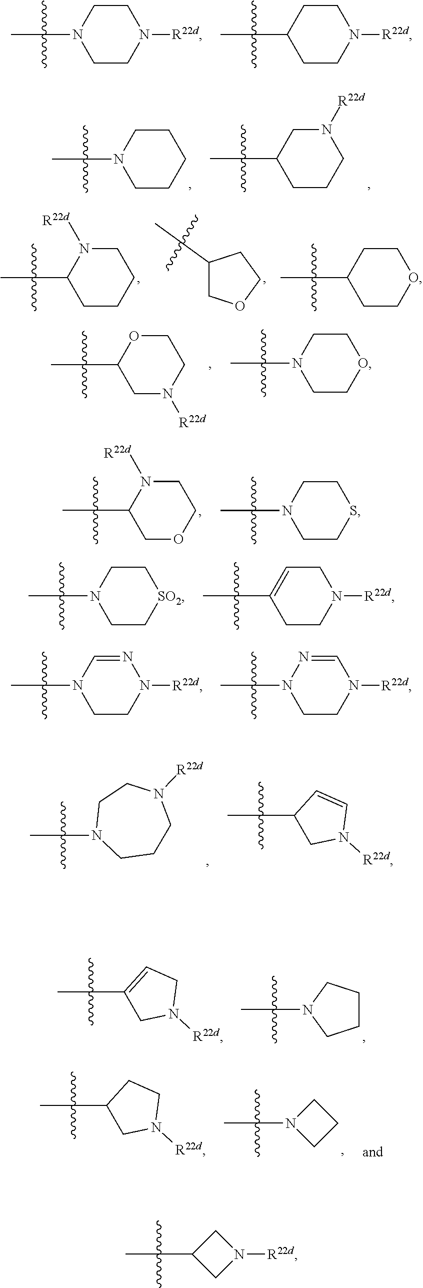

which is unsubstituted or substituted with one or more of R²², and
wherein
R^{22d} is selected from H or R²²; and

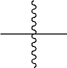

indicates a point of covalent attachment to Z.

In an embodiment, Cy² is selected from

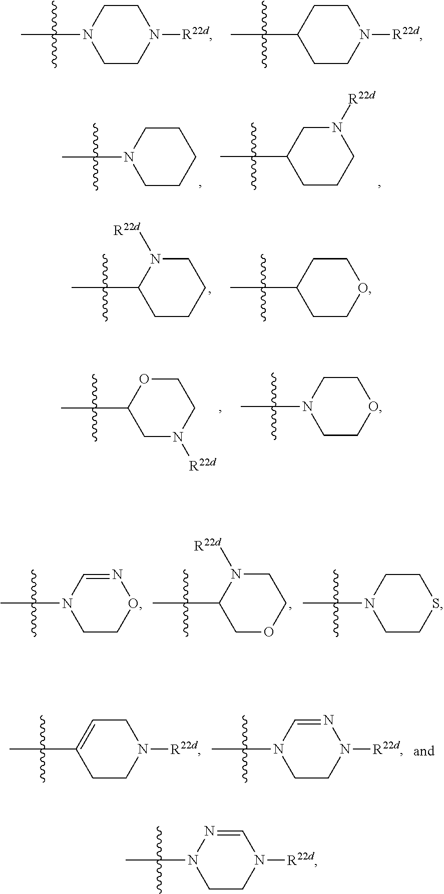

which is unsubstituted or substituted with one or more of R²², and
wherein R^{22d} is selected from H or R²²; and

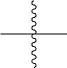

indicates a point of covalent attachment to Z.

In an embodiment, Cy² is a bicyclic heterocycle and Cy² is unsubstituted or substituted with one or more of R²². In an embodiment, Cy² is a bridged bicyclic heterocycle and Cy² is unsubstituted or substituted with one or more of R²². In an embodiments, Cy² is a fused bicyclic heterocycle or a spirofused bicyclic heterocycle and Cy² is unsubstituted or substituted with one or more of R²². In an embodiment, Cy² is a fused bicyclic heterocycle which is unsubstituted or substituted with one of more of R²². In an embodiment, Cy² is a C₆-C₁₀ saturated bicyclic ring in which one or two of the ring carbon atoms is replaced with N, NH or NR²², depending on the valency requirements of the N, which is unsubstituted or substituted with one of more of R²⁰. In an embodiment, Cy² is azabicyclohexanyl, diazabicycloheptanyl or diazabicyclooctanyl and Cy² is unsubstituted or substituted with one or more of R²². In an embodiment, Cy² is bridged azabicyclohexanyl, bridged diazabicycloheptanyl or bridged diazabicyclooctanyl. In an embodiment, Cy² is selected from the following structures

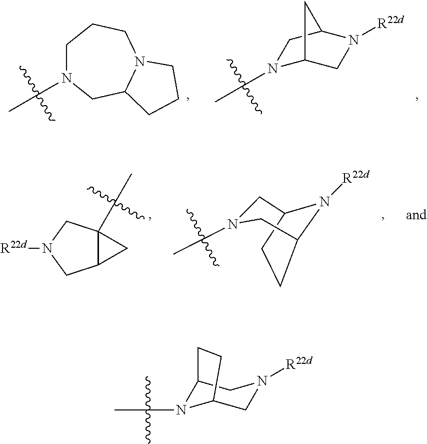

which is unsubstituted or is substituted with one or more of R²², and
wherein R^{22d} is selected from H or R²², and

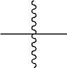

indicates a point of covalent attachment to Cy¹

In an embodiment, Cy² is selected from the following structures

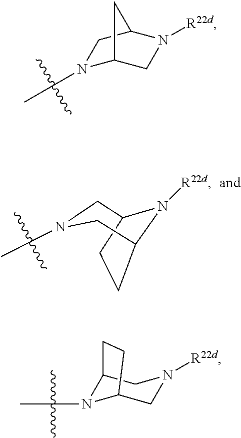

which is unsubstituted or is substituted with one or more of R²², and
wherein R^{22d} is selected from H or R²², and

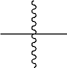

indicates a point of covalent attachment to Z. In an embodiment, Cy² is

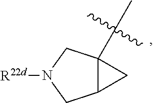

which is unsubstituted with one or more of R²² wherein R^{22d} is selected from H or R²², and

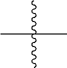

indicates a point of covalent attachment to Z.

In an embodiment, Cy² is selected from tetrahydrofuropyrrolyl, hexapyrazinooxazinyl, hexahydropyrrolopyrazinyl and hexahydropyrrolodiazepiny and Cy² is unsubstituted or substituted with one or more of R²². In an embodiment, Cy² is selected from

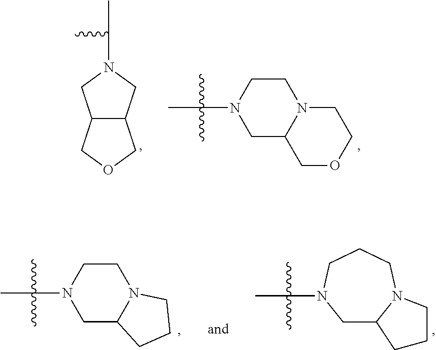

which unsubstituted or substituted with one or more of R²²; and
wherein

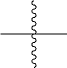

indicates a point of covalent attachment to Z.

In an embodiment, Cy² is spirofused bicyclic heterocycle and Cy² is unsubstituted or substituted with one to four of R²². In an embodiment, Cy² is selected from

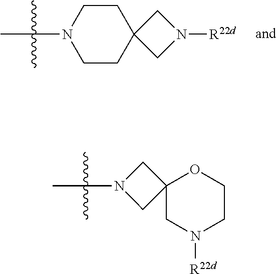

which is unsubstituted or substituted with one to four of R²²; and
wherein R^{22d} is selected from H and R²², and

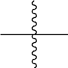

indicates a point of covalent attachment to Z.

It is appreciated by a person skilled in the art that when Z is absent, then

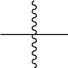

in the structures of Cy² indicates a point of covalent attachment to Cy¹.

In an embodiment, Cy² is unsubstituted. In an embodiment, Cy² is substituted with one to three of R²². In an embodiment, Cy² is substituted with one or two of R²². In an embodiment, Cy² is substituted with one of R²².

In an embodiment, each R²² is independently selected from F, Cl, ═O, CN, OH, C₁₋₄alkyl, C₂₋₄alkenyl, C₂₋₄alkynyl, C₃₋₁₀cycloalkyl, C₃₋₁₀heterocycloalkyl, C₆₋₁₁aryl, C₅₋₁₄ heteroaryl, C₁₋₄alkyleneC₃₋₁₀cycloalkyl, C₁₋₄alkyleneC₆₋₁₁aryl, C₁₋₄alkyleneC₅₋₁₄heteroaryl, C₁₋₄alkyleneC₃₋₁₀heterocycloalkyl, OC₁₋₄alkyl, OC₂₋₄alkenyl, OC₂₋₄alkynyl, C₁₋₄alkyleneOR²¹, OC₁₋₄alkyleneOR²¹, C₁₋₄alkyleneNR²⁴R²⁵, OC₁₋₄alkyleneNR²⁴R²⁵, SC₁₋₄alkyl, SC₂₋₄alkenyl, SC₂₋₄alkynyl, C(O)C₁₋₄alkyl, C(O)C₂₋₄alkenyl, C(O)C₂₋₄alkynyl, C(O)C₃₋₁₀cycloalkyl, C(O)C₃₋₁₀heterocycloalkyl, C(O)C₆₋₁₁aryl, C(O)C₅₋₁₄heteroaryl, C(O)C₁₋₄alkyleneC₃₋₁₀cycloalkyl, C(O)C₁₋₄alkyleneC₃₋₁₀heterocycloalkyl, C(O)C₁₋₄alkyleneC₆₋₁₁aryl, C(O)C₁₋₄alkyleneC₅₋₁₄ heteroaryl, C(O)C₁₋₄alkylenylOR²³, C(O)C₁₋₄alkyleneNR²⁴R²⁵, C(O)C₁₋₄alkyleneOC₁₋₄ alkyleneNR²⁴R²⁵, C(O)NR²⁴R²⁵, CO₂C₁₋₆alkyl, CO₂C₂₋₄alkenyl, CO₂C₂₋₄alkynyl, CO₂C₁₋₄alkyleneOR²³, CO₂C₁₋₄alkyleneOC₁₋₄alkyleneNR²⁴R²⁵, NR²⁴R²⁵, NR²⁶C₃₋₁₀cycloalkyl, NR²⁶C₃₋₁₀heterocycloalkyl, NR²⁶C₆₋₁₁aryl, NR²⁶C₅₋₁₄heteroaryl, NR²⁶C₁₋₄alkyleneC₃₋₁₀cycloalkyl, NR²⁶C₁₋₄alkyleneC₃₋₁₀heterocycloalkyl, NR²⁶C₁₋₄alkyleneC₆₋₁₁aryl, NR²⁶C₁₋₄alkyleneC₅₋₁₄ heteroaryl, NR²⁶C₁₋₄alkyleneOR²³, NR²⁶SO₂C₁₋₄alkyl, SO₂C₁₋₆alkyl, SO₂C₂₋₄alkenyl, SO₂C₂₋₄alkynyl, and SO₂NR²⁴R²⁵, and alkyl, alkenyl, alkynyl, alkylene, aryl, heteroaryl, heterocycloalkyl and cycloalkyl groups of R²² are optionally substituted with one to three of R²⁷. In an embodiment, each R²² is independently selected from F, Cl, ═O, CN, OH, C₁₋₄alkyl, C₂₋₄alkenyl, C₂₋₄alkynyl, C₃₋₁₀cycloalkyl, C₃₋₁₀heterocycloalkyl, C₁₋₄alkyleneC₃₋₁₀cycloalkyl, C₁₋₄alkyleneC₃₋₁₀heterocycloalkyl, OC₁₋₄alkyl, OC₂₋₄alkenyl, OC₂₋₄alkynyl, C₁₋₄alkyleneOR²¹, OC₁₋₄alkyleneOR²¹, C₁₋₄alkyleneNR²⁴R²⁵, OC₁₋₄alkyleneNR²⁴R²⁵, SC₁₋₄alkyl, SC₂₋₄alkenyl, SC₂₋₄alkynyl, C(O)C₁₋₄alkyl, C(O)C₂₋₄alkenyl, C(O)C₂₋₄alkynyl, C(O)C₃₋₁₀cycloalkyl, C(O)C₃₋₁₀heterocycloalkyl, C(O)C₁₋₄alkyleneC₃₋₁₀cycloalkyl, C(O)C₁₋₄alkyleneC₃₋₁₀heterocycloalkyl, C(O)C₁₋₄alkylenylOR²³, C(O)C₁₋₄alkyleneNR²⁴R²⁵, C(O)C₁₋₄alkyleneOC₁₋₄alkyleneNR²⁴R²⁵, C(O)NR²⁴R²⁵, CO₂C₁₋₆alkyl, CO₂C₂₋₄alkenyl, CO₂C₂₋₄alkynyl, CO₂C₁₋₄alkyleneOR²³, CO₂C₁₋₄alkyleneOC₁₋₄alkyleneNR²⁴R²⁵, NR²⁴R²⁵, NR²⁶C₃₋₁₀cycloalkyl, NR²⁶C₃₋₁₀heterocycloalkyl, NR²⁶C₁₋₄alkyleneC₃₋₁₀cycloalkyl, NR²⁶C₁₋₄alkyleneC₃₋₁₀heterocycloalkyl, NR²⁶C₁₋₄alkyleneOR²³, NR²⁶SO₂C₁₋₄alkyl, SO₂C₁₋₆alkyl, SO₂C₂₋₄alkenyl, SO₂C₂₋₄alkynyl, and SO₂NR²⁴R²⁵, and alkyl, alkenyl, alkynyl, alkylene and cycloalkyl groups of R²² are optionally substituted with one to three of R²⁷.

In an embodiment, each R²² is independently selected from F, Cl, ═O, CN, OH, NO₂, C₁₋₄alkyl, C₃₋₁₀cycloalkyl, C₃₋₁₀heterocycloalkyl, C₆₋₁₁aryl, C₅₋₁₀heteroaryl, C₁₋₄alkyleneC₃₋₁₀cycloalkyl, C₁₋₄alkyleneC₃₋₁₀heterocycloalkyl, C₁₋₄alkyleneC₆₋₁₁aryl, C₁₋₄alkyleneC₅₋₁₄heteroaryl, OC₁₋₄alkyl, C₁₋₄alkyleneOR²³, OC₁₋₄alkyleneOR²³, C₁₋₄alkyleneNR²⁴R²⁵, OC₁₋₄alkyleneNR²⁴R²⁵, SC₁₋₄alkyl, C(O)C₁₋₄alkyl, C(O)C₃₋₁₀cycloalkyl, C(O)C₃₋₁₀heterocycloalkyl, C(O)C₆₋₁₁aryl, C(O)C₅₋₁₀heteroaryl, C(O)C₁₋₄alkyleneC₃₋₁₀cycloalkyl, C(O)C₁₋₄alkyleneC₃₋₁₀heterocycloalkyl, C(O)C₁₋₄alkyleneC₆₋₁₁aryl, C(O)C₁₋₄alkyleneC₅₋₁₀heteroaryl, C(O)C₁₋₄alkyleneOR²³, C(O)C₁₋₄alkyleneNR²⁴R²⁵, C(O)C₁₋₄alkyleneOC₁₋₄alkyleneNR²⁴R²⁵, C(O)NR²⁴R²⁵, CO₂C₁₋₆alkyl, CO₂C₁₋₄alkyleneOR²³, CO₂C₁₋₄alkyleneOC₁₋₄alkyleneNR²⁴R²⁵, NR²⁴R²⁵, NR²⁶C₃₋₁₀cycloalkyl, NR²⁶C₃₋₁₀heterocycloalkyl, NR²⁶C₆₋₁₁aryl, NR²⁶C₅₋₁₀heteroaryl, NR²⁶C₁₋₄alkyleneC₃₋₁₀cycloalkyl, NR²⁶C₁₋₄alkyleneC₃₋₁₀heterocycloalkyl, NR²⁶C₁₋₄alkyleneC₆₋₁₁aryl, NR²⁶C₁₋₄alkyleneC₅₋₁₀heteroaryl, NR²⁶C₁₋₄alkyleneOR²³, NR²⁶SO₂C₁₋₄alkyl, SO₂C₁₋₆alkyl, and SO₂NR²⁴R²⁵, and alkyl, alkenyl, alkynyl, alkylene, aryl, heteroaryl, heterocycloalkyl and cycloalkyl groups of R²² are optionally substituted with one to three of R²⁷. In an embodiment, each R²² is independently selected from F, Cl, CN, OH, NO₂, C₁₋₄alkyl, C₃₋₁₀cycloalkyl, C₃₋₁₀heterocycloalkyl, C₁₋₄alkyleneC₃₋₁₀cycloalkyl, C₁₋₄alkyleneC₃₋₁₀heterocycloalkyl, OC₁₋₄alkyl, C₁₋₄alkyleneOR²³, OC₁₋₄alkyleneOR²³, C₁₋₄alkyleneNR²⁴R²⁵, OC₁₋₄alkyleneNR²⁴R²⁵, SC₁₋₄alkyl, C(O)C₁₋₄alkyl, C(O)C₃₋₁₀cycloalkyl, C(O)C₃₋₁₀heterocycloalkyl, C(O)C₁₋₄alkyleneC₃₋₁₀cycloalkyl, C(O)C₁₋₄alkyleneC₃₋₁₀heterocycloalkyl, C(O)C₁₋₄alkyleneOR²³, C(O)C₁₋₄alkyleneNR²⁴R²⁵, C(O)C₁₋₄alkyleneOC₁₋₄alkyleneNR²⁴R²⁵, C(O)NR²⁴R²⁵, CO₂C₁₋₆alkyl, CO₂C₁₋₄alkyleneOR²³, CO₂C₁₋₄alkyleneOC₁₋₄alkyleneNR²⁴R²⁵, NR²⁴R²⁵, NR²⁶C₃₋₁₀cycloalkyl, NR²⁶C₃₋₁₀heterocycloalkyl, NR²⁶C₁₋₄alkyleneC₃₋₁₀cycloalkyl, NR²⁶C₁₋₄alkyleneC₃₋₁₀heterocycloalkyl, NR²⁶C₁₋₄alkyleneOR²³, NR²⁶SO₂C₁₋₄alkyl, SO₂C₁₋₆alkyl, and SO₂NR²⁴R²⁵, and alkyl, alkenyl, alkynyl, alkylene and cycloalkyl groups of R²² are optionally substituted with one to three of R²⁷. In an embodiment, one R²² is ═O.

In an embodiment, each R²² is independently selected from F, Cl, ═O, CN, OH, NO₂, C₁₋₄alkyl, C₃₋₁₀cycloalkyl, C₃₋₁₀heterocycloalkyl, C₁₋₆-aryl, C₅₋₁₀heteroaryl, C₁₋₂alkyleneC₃₋₁₀cycloalkyl, C₁₋₂alkyleneC₃₋₁₀heterocycloalkyl, C₁₋₄alkyleneC₆₋₁₁aryl, C₁₋₄alkyleneC₅₋₁₄heteroaryl, OC₁₋₄alkyl, C₁₋₄alkyleneOR²¹, C(O)C₁₋₄alkyl, C(O)C₃₋₁₀cycloalkyl, C(O)C₃₋₁₀heterocycloalkyl, (O)C₆₋₁₁aryl, C(O)C₅₋₁₀heteroaryl, C(O)C₁₋₄alkyleneC₃₋₁₀cycloalkyl, C(O)C₁₋₄alkyleneC₃₋₁₀heterocycloalkyl, C(O)C₁₋₄alkyleneC₆₋₁₁aryl, C(O)C₁₋₄alkyleneC₅₋₁₀heteroaryl, C(O)C₁₋₄alkyleneOR²³, C(O)C₁₋₄alkyleneNR²⁴R²⁵, C(O)C₁₋₄alkyleneOC₁₋₄ alkyleneNR²⁴R²⁵, CO₂C₁₋₆alkyl, CO₂C₁₋₄alkyleneOR²³, NR²⁴R²⁵, NR²⁶C₃₋₁₀cycloalkyl, NR²⁶C₃₋₁₀heterocycloalkyl, NR²⁶C₆₋₁₁aryl, NR²⁶C₅₋₁₀heteroaryl, NR²⁶C₁₋₂alkyleneC₃₋₁₀cycloalkyl, NR²⁶C₁₋₂alkyleneC₃₋₁₀heterocycloalkyl, NR²⁶C₁₋₄alkyleneC₆₋₁₁aryl, NR²⁶C₁₋₄alkyleneC₅₋₁₀heteroaryl, NR²⁶C₁₋₄alkyleneOR²³, NR²⁶SO₂C₁₋₄alkyl, and SO₂C₁₋₆alkyl, and alkyl, alkenyl, alkynyl, alkylene, aryl, heteroaryl, heterocycloalkyl and cycloalkyl groups of R²² are optionally substituted with one to three of R²⁷. In an embodiment, each R²² is independently selected from F, Cl, CN, OH, NO₂, C₁₋₄alkyl, C₃₋₁₀cycloalkyl, C₃₋₁₀heterocycloalkyl, C₁₋₂alkyleneC₃₋₁₀cycloalkyl, C₁₋₂alkyleneC₃₋₁₀heterocycloalkyl, OC₁₋₄alkyl, C₁₋₄alkyleneOR²¹, C(O)C₁₋₄alkyl, C(O)C₃₋₁₀cycloalkyl, C(O)C₃₋₁₀heterocycloalkyl, C(O)C₁₋₄alkyleneC₃₋₁₀cycloalkyl, C(O)C₁₋₄alkyleneC₃₋₁₀heterocycloalkyl, C(O)C₁₋₄alkyleneOR²³, C(O)C₁₋₄alkyleneNR²⁴R²⁵, C(O)C₁₋₄alkyleneOC₁₋₄alkyleneNR²⁴R²⁵, CO₂C₁₋₆alkyl, CO₂C₁₋₄alkyleneOR²³, NR²⁴R²⁵, NR²⁶C₃₋₁₀cycloalkyl, NR²⁶C₃₋₁₀heterocycloalkyl, NR²⁶C₁₋₂alkyleneC₃₋₁₀cycloalkyl, NR²⁶C₁₋₂alkyleneC₃₋₁₀heterocycloalkyl, NR²⁶C₁₋₄alkyleneOR²³, NR²⁶SO₂C₁₋₄alkyl, and SO₂C₁₋₆alkyl, and alkyl, alkenyl, alkynyl, alkylene and cycloalkyl groups of R²² are optionally substituted with one to three of R²⁷.

In an embodiment, each R²⁷ is independently selected from F, Cl, CN, C₁₋₄alkyl and NR^{27a}R^{27b}. In an embodiment, each R²⁷ is independently selected from F, C₁₋₄alkyl and NR^{27a}R^{27b}. In an embodiment, at least one R²⁷ is F. In an embodiment, one of R²⁷ is NR^{27a}R^{27b}.

In an embodiment, R^{27a} and R^{27b} are independently selected from H and C₁₋₄alkyl. In an embodiment, R^{27a} and R^{27b} are independently selected from H, CH₃, CH₂CH₃, CF₂H, CF₃, CFH₂, CH₂CF₂H and CH₂CF₃. In an embodiment, R^{27a} and R^{27b} are independently selected from H, CF₃ and CH₃. In an embodiment, R^{27a} and R^{27b} are independently selected from H and CH₃.

In an embodiment, R²³ is selected from H, C₁₋₄alkyl, C₁₋₄alkyleneOC₁₋₆alkyl, C₃₋₁₁cycloalkyl, C₃₋₁₀heterocycloalkyl, C₂₋₄alkenyl, C₂₋₄alkynyl, C₁₋₄alkyleneC₃₋₁₀cycloalkyl, and C₁₋₄alkyleneC₃₋₁₀heterocycloalkyl. In an embodiment, R²³ is selected from H and C₁₋₄alkyl. In an embodiment, R²³ is selected from C₁₋₄alkyleneOC₁₋₆alkyl, C₁₋₄alkyleneC₃₋₁₀cycloalkyl, and C₁₋₄alkyleneC₃₋₁₀heterocycloalkyl.

In an embodiment, R²⁴ is selected from H and C₁₋₄alkyl.

In an embodiment, R²⁵ is selected from H, C₁₋₄alkyl, C₁₋₄alkyleneOC₁₋₄alkyl, C₃₋₁₀cycloalkyl, C₃₋₁₀heterocycloalkyl, C₁₋₄alkyleneC₃₋₁₀cycloalkyl, and C₁₋₄alkyleneC₃₋₁₀heterocycloalkyl.

In an embodiment, R²⁴ and R²⁵ are each independently selected from H and C₁₋₄alkyl. In an embodiment, R²⁴ and R²⁵ are joined to form, together with the atom therebetween, a 4- to 6-membered saturated or unsaturated ring, optionally containing one additional heteromoiety selected from N, NR²⁸, O, S, S(O), and SO₂, and optionally substituted with one to three of halo and C₁₋₆alkyl.

In an embodiment, R²³, R²⁶ and R²⁸ are independently selected from H and C₁₋₄alkyl.

In an embodiment, each R²² is independently selected from F, Cl, ═O, CN, OH, CH₃, CH₂CH₃, CH(CH₃)₂, C(CH₃)₃, CF₂H, CF₃, CFH₂, CH₂CFH₂, CH₂CF₂H, CH₂CH₂CF₂H, CH₂CH₂CH₂CF₂H, CH₂CF₃, CH₂CH₂CF₃, CH₂CH₂CH₂CF₃, C₃₋₆cycloalkyl, C₃₋₆heterocycloalkyl, phenyl, C₅₋₁₄heteroaryl, C₁₋₄alkyleneC₃₋₆cycloalkyl, C₁₋₄alkyleneC₃₋₆heterocycloalkyl, C₁₋₄alkylenephenyl, C₁₋₄alkyleneC₅₋₁₄heteroaryl, OC₁₋₄alkyl, C₁₋₄alkyleneOH, C₁₋₄alkyleneOCH₃, C(O)C₁₋₄alkyl, C(O)C₃₋₆cycloalkyl, C(O)C₃₋₆heterocycloalkyl, C(O)phenyl, C(O)C₅₋₁₄heteroaryl, C(O)C₁₋₂alkyleneC₃₋₆cycloalkyl, C(O)C₁₋₂alkyleneC₃₋₁₀heterocycloalkyl, C(O)C₁₋₂alkylenephenyl, C(O)C₁₋₂alkyleneC₅₋₁₄heteroaryl, C(O)C₁₋₄alkyleneOCH₃, C(O)C₁₋₄alkyleneOH, C(O)C₁₋₄alkyleneNH₂, C(O)C₁₋₄alkyleneN(CH₃)₂, C(O)C₁₋₄alkyleneOC₁₋₄alkyleneN(CH₃)₂, CO₂C₁₋₆alkyl, CO₂C₁₋₄alkyleneOC₁₋₆alkyl, NHC₁₋₄alkyl, NC₁₋₄alkylC₁₋₄alkyl, NHC₁₋₂alkyleneC₃₋₁₀cycloalkyl, NHC₁₋₂alkylenephenyl, NHC₁₋₂alkyleneC₅₋₁₄heteroaryl, N(CH₃)C₁₋₂alkyleneC₃₋₁₀cycloalkyl, N(CH₃)C₁₋₂alkyleneC₃₋₁₀heterocycloalkyl, N(CH₃)C₁₋₂alkylenephenyl, N(CH₃)C₁₋₂alkyleneC₅₋₁₄heteroaryl, NHC₁₋₂alkyleneC₃₋₁₀heterocycloalkyl, NR²⁶C₁₋₄alkyleneOH, NR²⁶C₁₋₄alkyleneOCH₃, NHSO₂C₁₋₄alkyl, NCH₃SO₂C₁₋₄alkyl, and SO₂C₁₋₆alkyl, and alkyl, alkylene, phenyl, heteroaryl, heterocycloalkyl and cycloalkyl groups of R²² are optionally substituted with one to three of R²⁷, and each R²⁷ is independently selected from F, C and C₁₋₄alkyl. In an embodiment, each R²² is independently selected from F, C, ═O, CN, OH, CH₃, CH₂CH₃, CH(CH₃)₂, C(CH₃)₃, CF₂H, CF₃, CFH₂, CH₂CFH₂, CH₂CF₂H, CH₂CF₃, CH₂CH₂CF₃, CH₂CH₂CH₂CF₃, C₃₋₆cycloalkyl, C₃₋₆heterocycloalkyl, C₁₋₄alkyleneC₃₋₆cycloalkyl, C₁₋₄alkyleneC₃₋₆heterocycloalkyl, OC₁₋₄alkyl, C₁₋₄alkyleneOH, C₁₋₄alkyleneOCH₃, C(O)C₁₋₄alkyl, C(O)C₃₋₆cycloalkyl, C(O)C₃₋₆heterocycloalkyl, C(O)C₁₋₂alkyleneC₃₋₆cycloalkyl, C(O)C₁₋₂alkyleneC₃₋₁₀heterocycloalkyl, C(O)C₁₋₄alkyleneOCH₃, C(O)C₁₋₄alkyleneOH, C(O)C₁₋₄alkyleneNH₂, C(O)C₁₋₄alkyleneN(CH₃)₂, C(O)C₁₋₄alkyleneOC₁₋₄alkyleneN(CH₃)₂, CO₂C₁₋₆alkyl, CO₂C₁₋₄alkyleneOC₁₋₆alkyl, NHC₁₋₄alkyl, NC₁₋₄alkylC₁₋₄alkyl, NHC₁₋₂alkyleneC₃₋₁₀cycloalkyl, NCH₃C₁₋₂alkyleneC₃₋₁₀cycloalkyl, NCH₃Cl₁₋₂alkyleneC₃₋₁₀heterocycloalkyl, NHC₁₋₂alkyleneC₃-10heterocycloalkyl, NR²⁶C₁₋₄alkyleneOH, NR²⁶C₁₋₄alkyleneOCH₃, NHSO₂C₁₋₄alkyl, NCH₃SO₂C₁₋₄alkyl, and SO₂C₁₋₆alkyl, and alkyl, alkylene and cycloalkyl groups of R²² are optionally substituted with one to three of R²⁷, and each R²⁷ is independently selected from F, Cl and C₁₋₄alkyl. In an embodiment, each R²² is independently selected from CH₂CH₂CF₂H and CH₂CH₂CH₂CF₂H.

In an embodiment, each R²² is independently selected from C₃₋₆cycloalkyl, C₃₋₆heteroycloalkyl, C₁₋₄alkyleneC₃₋₆cycloalkyl and C₁₋₄alkyleneC₃₋₆heterocycloalkyl. In an embodiment, each R²² is independently selected from C₃₋₅cycloalkyl, C₃₋₆heteroycloalkyl, C₁₋₄alkyleneC₃₋₅cycloalkyl, and C₁₋₄alkyleneC₃₋₅heterocycloalkyl. In an embodiments, all cycloalkyl groups of R²² are optionally substituted with one to three of R²⁷.

In an embodiment, each R²² is independently selected from cyclopropyl, cyclobutyl and cyclopentyl. In an embodiment, each R²² is independently selected from cyclopropyl and cyclobutyl. In an embodiments, each of the cyclopropyl, cyclobutyl and cyclopentyl are optionally substituted with one to three of R²⁷.

In an embodiment, each R²² is independently selected from C₁₋₄alkylenecyclopropyl, C₁₋₄alkylenecyclobutyl and C₁₋₄alkylenecyclopentyl. In an embodiment all cycloalkyl groups of R²² are optionally substituted with one to three of R²⁷. In an embodiment, each R²² is independently selected from C₁₋₄alkylenecyclopropyl and C₁₋₄alkyleneC₃cyclobutyl. In an embodiment, each R²² is independently selected from C₁₋₄alkylenecyclopropyl, C₁₋₄alkyleneC₃cyclobutyl and C₁₋₄alkylenecyclopentyl and all cycloalkyl groups of R²² are optionally substituted with one to three of R²⁷. In an embodiment, one R²² is C₁₋₃alkylenecyclopropyl. In an embodiment R²² is C₁₋₃alkylenecyclopropyl optionally substituted with one to three of R²⁷. In an embodiment, one R²² is C₁₋₃alkylenecyclopropyl selected from

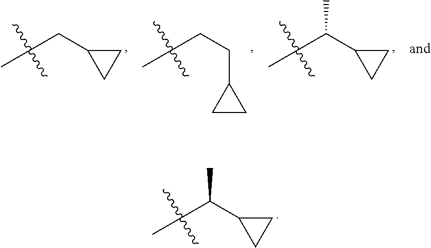

In an embodiment, one R²⁰ is selected from CH₃, CH₂CH₃, CF₂H, CF₃, CFH₂, CH₂CF₂H, CH₂CH₂CF₂H, CH₂CH₂CH₂CF₂H and CH₂CF₃. In an embodiment, each R²² is independently selected from CH₃, CH₂CH₃, CF₂H, CF₃, CFH₂, CH₂CF₂H, and CH₂CF₃. In an embodiment, each R²² is independently selected from CH₃, CH₂CH₃, CH(CH₃)₂. In an embodiment, each R²² is independently selected from CH₂CH₃ and CH(CH₃)₂. In an embodiment, each R²² is independently selected from CH₂CH₃ and CH(CH₃)₂ which are optionally fluoro-substituted. In an embodiment, one R²² is selected CF₂H, CH₂CF₂H, CH₂CH₂CF₂H, and CH₂CH₂CH₂CF₂H.

In an embodiment, one of R²² is selected from phenyl, C₆₋₁₁heteroaryl, C₁₋₄alkyleneC₆₋₁₁aryl and C₁₋₄alkyleneC₅₋₁₄heteroaryl, and aryl and heteroaryl groups of R²² are optionally substituted with one to three of R²⁷. In an embodiment, one of R²² is selected from phenyl, C₅₋₇heteroaryl, C₁₋₄alkylenephenyl and C₁₋₄alkyleneC₅₋₇heteroaryl, and all aryl and heteroaryl and cycloalkyl groups of R²² are optionally substituted with one to three of R²⁷. In an embodiment, one of R²² is selected from phenyl and C₁₋₂alkylenephenyl optionally substituted with one to three of R²⁷. In an embodiment, one of R²⁰ is selected from C₅₋₇ heteroaryl and C₁₋₆alkyleneC₅₋₇heteroaryl, and all heteroaryl groups of R²² are optionally substituted with one to three of R²⁷. In an embodiment, the heteroaryl in C₅₋₇heteroaryl and C₁₋₆alkyleneC₅₋₇heteroaryl, of R²⁷ are selected from pyrrolyl, furanyl, thiophenyl, imidazolyl, pyrazolyl, oxathiolyl, isoxathiolyl, oxaxolyl, isoxazolyl, thiazoyl, isothiazolyl, triazolyl and tetrazolyl. In an embodiment, the heteroaryl in C₅₋₇heteroaryl and C₁₋₆alkyleneC₅₋₇heteroaryl, of R²² is triazolyl. In an embodiment, the triazole is 1,2,3 trizole or 1, 2, 4-triazole.

In an embodiment, one of R²² is selected from C₃₋₆heteroycloalkyl and C₁₋₄ alkyleneC₃₋₆heterocycloalkyl. In an embodiment, the C₃₋₆heteroycloalkyl in the C₃₋₆heteroycloalkyl and C₁₋₄alkyleneC₃₋₆heterocycloalkyl of R²² is independently selected from oxetanyl, tetrahydrofuranyl and tetrahydropyranyl.

In an embodiment, one R²² is CO₂C₁₋₆alkyl. In an embodiment, one R²² is selected from CO₂CH₃, CO₂CH₂CH₃, CO₂CF₂H, CO₂CF₃, CO₂CFH₂, CO₂CH₂CF₂H, CO₂CH₂CF₃, CO₂CH₂CH₂F₂H, CO₂CH₂CH₂CH₂F₂H, CO₂CH(CH₃)₂, and CO₂CH₂CH(CH₃)₂. In an embodiment, one R²² is CO₂CH₃.

In an embodiment, one R²² is COC₁₋₆alkyl. In an embodiment, one R²² is selected from COCCH₃, COCH₂CH₃, COCF₂H, COCF₃, COCFH₂, COCH₂CF₂H, COCH₂CF₃, COCH₂CH₂F₂H, COCH₂CH₂CH₂F₂H, COCH(CH₃)₂, and COCH₂CH(CH₃)₂. an embodiment, one R²² is COCH₃.

In an embodiment, each R²² is independently selected from OH, F, Cl, CF₃, CF₂H, CH₃, CH₂CH₃, CH₂CF₂H, CH₂CH₂CF₂H, CH₂CH₂CH₂F₂H

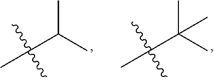

OC₁₋₄alkyl, SO₂C₁₋₄alkyl,

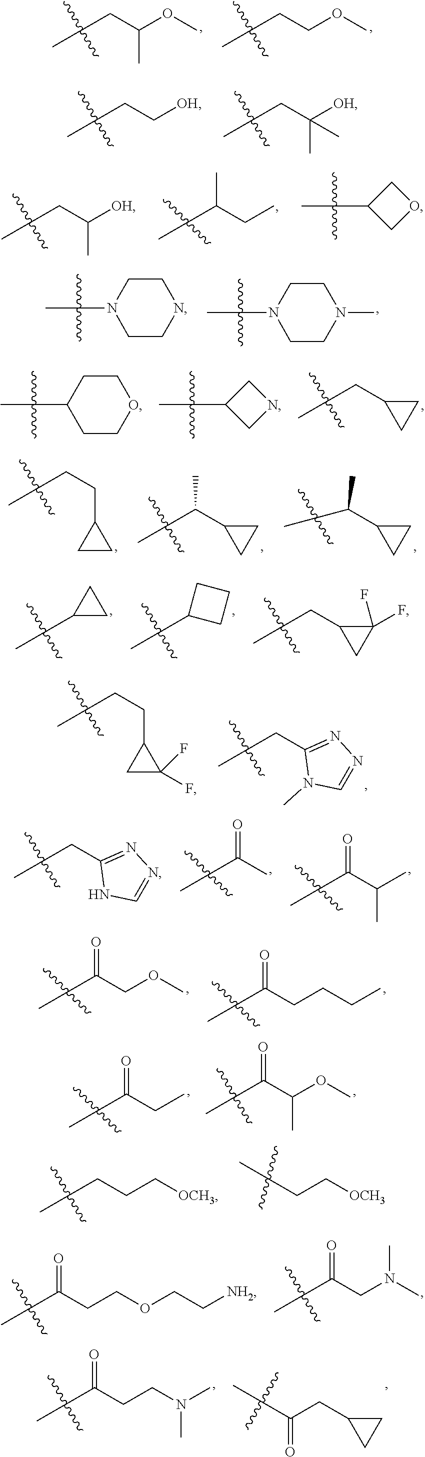

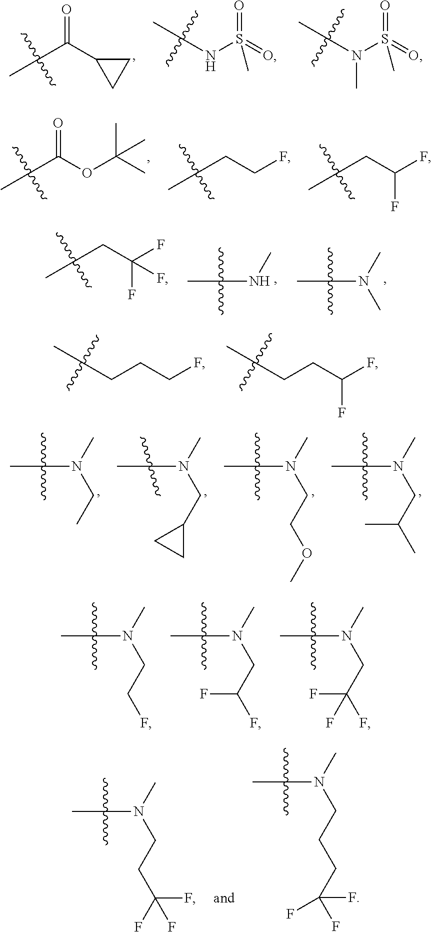

In an embodiment, each R²² is independently selected from OH, F, Cl, CF₃, CH₃, CH₂CH₃,

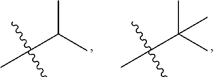

OC₁₋₄alkyl, SO₂C₁₋₄alkyl,

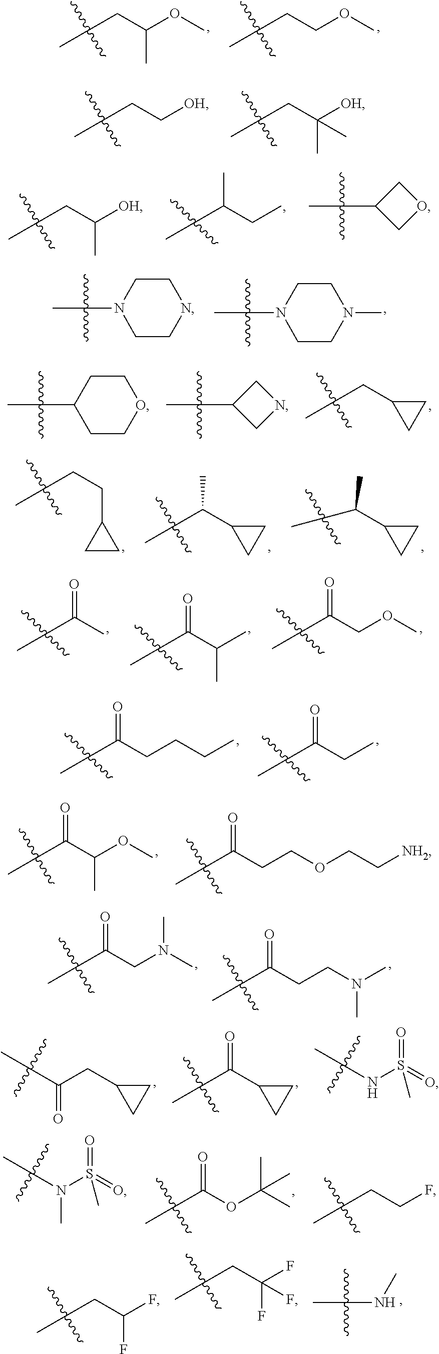

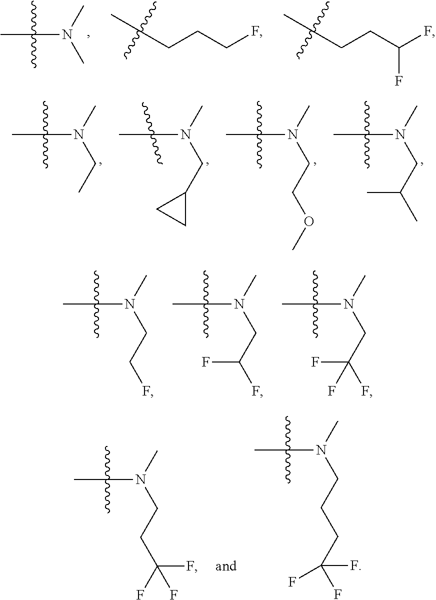

In an embodiment, Cy² is pyrrolidinyl, piperidinyl, piperazinyl or morpholinyl, and each R²² is independently CH₃, CH₂CH₃, CF₂H, CF₃, CFH₂, CH₂CF₂H, and CH₂CF₃. In an embodiment, Cy² is pyrrolidinyl, piperidinyl, piperazinyl or morpholinyl, and each R²² is independently CH₂CH₃ or CH(CH₃)₂.

In an embodiment, Cy² is pyrrolidinyl, piperidinyl, piperazinyl or morpholinyl, and one R²² is C₁₋₃alkylenecyclopropyl.

In an embodiment, the compound of Formula (I) is a compound of Formula (I-A) or (I-B) or a pharmaceutically acceptable salt, solvate and/or prodrug thereof

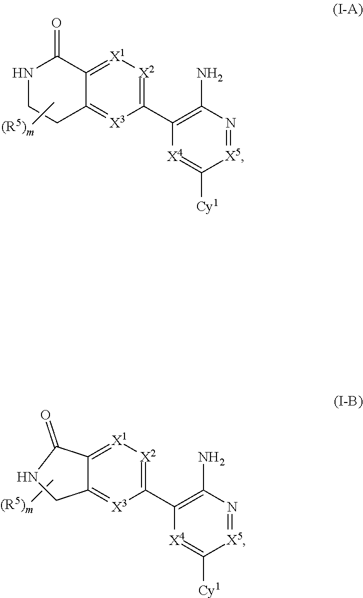

wherein R⁵, X¹, X², X³, X⁴, X⁵ and Cy¹ are as defined in Formula (I) above; and
m is an integer selected from 0 to 4,
wherein all available hydrogen atoms are optionally substituted with a fluorine atom.

In an embodiment, the compound of Formula (I) is a compound of Formula (I-C) or a pharmaceutically acceptable salt, solvate and/or prodrug thereof

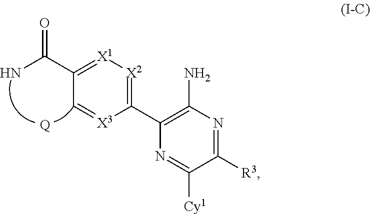

wherein Q, X¹, X², X³, and R³ and Cy¹ are as defined in Formula (I) above,
wherein all available hydrogen atoms are optionally substituted with a fluorine atom.

In an embodiment, the compound of Formula (I) is a compound of Formula (I-D), and (I-E) or a pharmaceutically acceptable salt, solvate and/or prodrug thereof

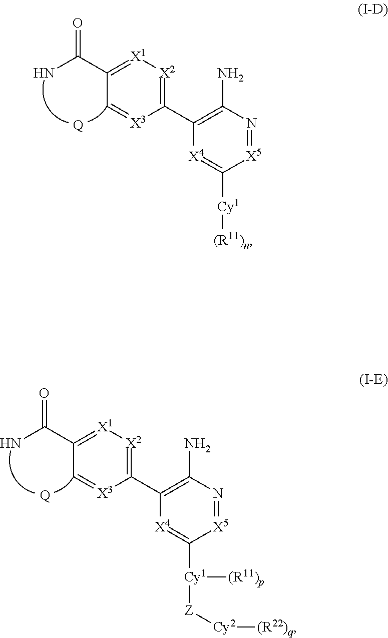

wherein Q, R¹¹, R²², X¹, X², X³, X⁴, X⁵, Z, Cy¹ and Cy² are as defined in Formula (I) above;
n is an integer selected from 0 to 5; and
p and q are integers independently selected from 0 to 4,
wherein all available hydrogen atoms are optionally substituted with a fluorine atom.

In an embodiment, the compound of Formula (I) is a compound of Formula (I-D), and (I-E) or a pharmaceutically acceptable salt, solvate and/or prodrug thereof

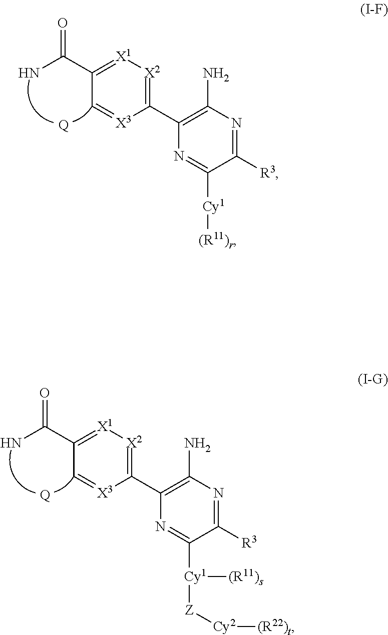

wherein Q, R³, R¹¹, R²², X¹, X², X³, Z, Cy¹ and Cy² are as defined in Formula (I) above;
r is an integer selected from 0 to 5; and
s and t are integers independently selected from 0 to 4,
wherein all available hydrogen atoms are optionally substituted with a fluorine atom.

In an embodiment, R³ is F

In an embodiment, the compound of Formula (I) is a compound of Formula (I-H) or a pharmaceutically acceptable salt, solvate and/or prodrug thereof

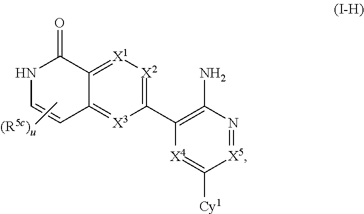

wherein R^{5c}, X¹, X², X³, X⁴, X⁵ and Cy¹ are as defined in Formula (I) above; and
u is an integer selected from 0 to 2,
wherein all available hydrogen atoms are optionally substituted with a fluorine atom.

In an embodiment, the compound of Formula (I) is a compound of Formula (I-J) or (I-K) or a pharmaceutically acceptable salt, solvate and/or prodrug thereof

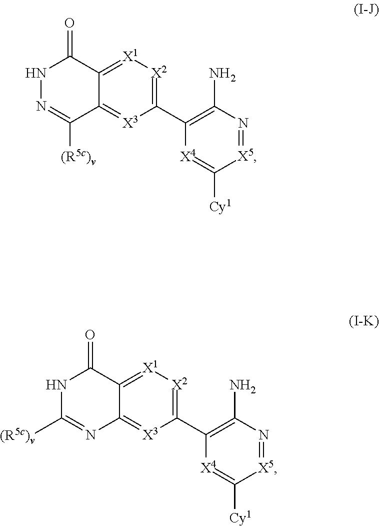

wherein R^{5c}, X¹, X², X³, X⁴, X⁵ and Cy¹ are as defined in Formula (I) above; and
v is an integer selected from 0 and 1,
wherein all available hydrogen atoms are optionally substituted with a fluorine atom.

In an embodiment, the compound of Formula (I) is a compound of Formula (I-L) or (I-M) or a pharmaceutically acceptable salt, solvate, and/or prodrug thereof:

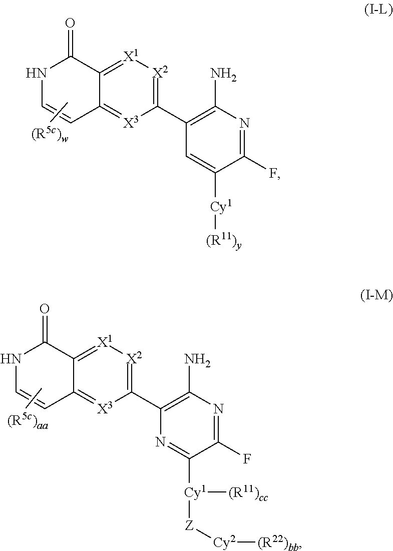

wherein R^{5c}, R¹¹, X¹, X², X³, Z, Cy¹ and Cy² are as defined in Formula (I) above;
w and aa are integers independently selected from 0 to 2,
y and bb are integers independently selected from 0 to 4, and
cc is an integer selected from 0 to 5.

In an embodiment, R^{5c} in the formula of (I-L) or (I-M) is selected from F, Cl, OH, C₁₋₄alkyl, OC₁₋₄alkyl and NR⁹R¹⁰. In an embodiment, one or two R^{5c} are selected from F, Cl, CH₃, CF₂H, CF₃, OCH₃, OCF₃, OCF₂H and NR⁹R¹⁰.

In an embodiment, the compound of Formula (I) is a compound of Formula (I-N) or (I-O) or a pharmaceutically acceptable salt, solvate, and/or prodrug thereof

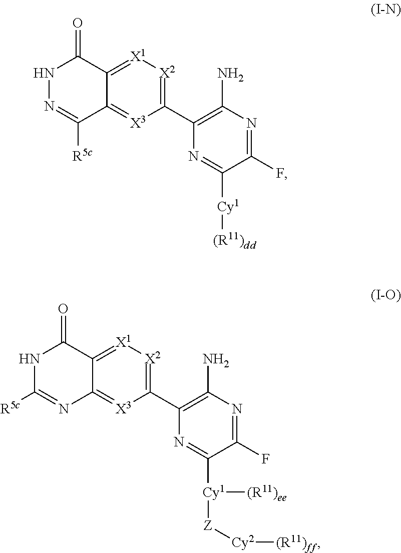

wherein R^{5c}, R¹¹, X¹, X², X³, Z, Cy¹ and Cy² are as defined in Formula (I) above;
dd and ee are integers independently selected from 0 to 4, and
ff is an integer selected from 0 to 5.

In an embodiment, the compound of Formula (I) is a compound of Formula (I-P) or (I-Q), or a pharmaceutically acceptable salt, solvate, and/or prodrug thereof

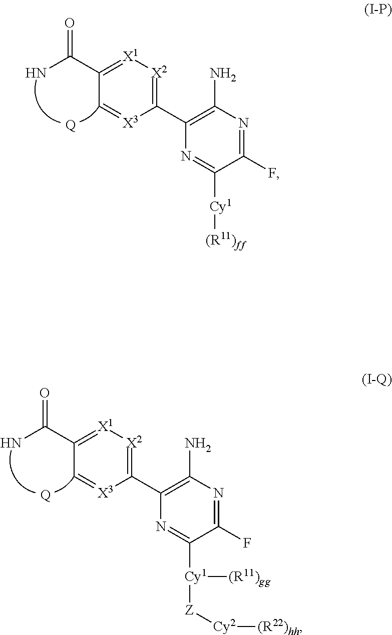

wherein Q, R²², X¹, X², X³, Z, Cy¹, and Cy² are as defined in Formula (I) above;
R¹¹ is selected from C₁₋₄alkyleneNR^{12a}R^{13a}, NR^{12a}R^{13a}, NR^{13a}COR¹², SO₂NR¹²R¹³, NR^{13a}C₁₋₄alkyleneNR¹²R¹³, C₃₋₇heterocycloalkyl, and C₁₋₄alkyleneC₃₋₇heterocycloalkyl, the latter four groups being optionally substituted with one to four of R¹⁵,
R¹², R¹², R¹³, R^{13a} and R¹⁵ is as defined in Formula (I) above;
ff is an integer selected from 1 to 5; and
gg is an integer selected from 1 to 4, and
hh is an integer selected from 0 to 5.

In an embodiment, ff is 1 or 2, suitably 1. In an embodiment, gg is 1 or 2, suitably 1. In an embodiment, one R¹¹ in the of Formula (I-P) or (I-Q) is selected from C₁₋₄alkyleneNR^{12a}R^{13a}, NR^{12a}R^{13a}, NR^{13a}COR¹², NR^{13a}C₁₋₄alkyleneNR¹²R¹³, C₃₋₇heterocycloalkyl, and C₁₋₄alkyleneC₃₋₇heterocycloalkyl, the latter two groups being optionally substituted with one to four of R¹⁵ as defined above for Formula (I). In an embodiment, one or two R¹¹ in the of Formula (I-P) or (I-Q) are selected from C₁₋₄alkyleneNR^{12a}R^{13a}, NR^{12a}R^{13a}, C₃₋₇heterocycloalkyl, and C₁₋₄alkyleneC₃₋₇heterocycloalkyl, the latter two groups being optionally substituted with one to four of R¹⁵ as defined above for Formula (I). In an embodiment, one R¹¹ in the of Formula (I-P) or (I-Q) is C₁₋₂alkyleneNR^{12a}R^{13a} as defined above for Formula (I). In an embodiment ff and gg are 1 or 2, suitably 1.

In an embodiment, the compound of Formula (I) is a compound of Formula (I-R) or (I-S) or a pharmaceutically acceptable salt, solvate, and/or prodrug thereof:

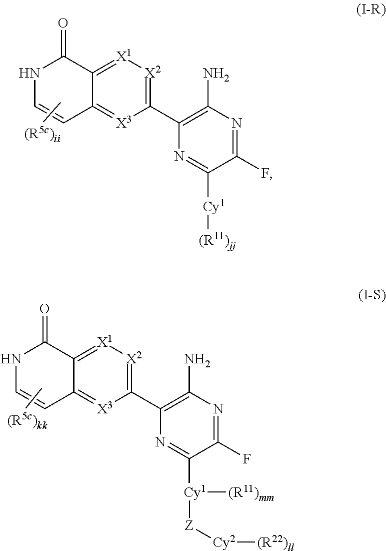

wherein R^{5c}, X¹, X², X³, Z, Cy¹ and Cy² are as defined in Formula (I) above;
R¹¹ is selected from C₁₋₄alkyleneNR^{12a}R^{13a}, NR^{12a}R^{13a}, NR^{13a}COR¹², SO₂NR¹²R¹³, NR^{13a}C₁₋₄alkyleneNR¹²R¹³, C₃₋₇heterocycloalkyl, and C₁₋₄alkyleneC₃₋₇heterocycloalkyl, the latter four groups being optionally substituted with one to four of R¹⁵
R¹², R¹², R¹³, R^{13a} and R¹⁵ are as defined in Formula (I) above;
ii and kk are integers independently selected from 0 to 2,
jj and mm are integers independently selected from 1 to 4, and
ll is an integer selected from 0 to 5.

In an embodiment, ii and kk are both 1, and R^{5c} in the formula of (I-R) or (I-S) is selected from F and Cl. In an embodiment, ii and kk are both 1, and R^{5c} in the formula of (I-R) or (I-S) is F.

In an embodiment, jj is 1 or 2, suitably 1. In an embodiment, II is 1 or 2, suitably 1. In an embodiment, one R¹¹ in the of Formula (I-R) or (I-S) is selected from C₁₋₄alkyleneNR^{12a}R^{13a}, NR^{12a}R^{13a}, NR^{13a}COR¹², NR^{13a}C₁₋₄alkyleneNR¹²R¹³, C₃₋₇heterocycloalkyl, and C₁₋₄alkyleneC₃₋₇heterocycloalkyl, the latter two groups being optionally substituted with one to four of R¹⁵ as defined above for Formula (I). In an embodiment, one or two R¹¹ in the of Formula (I-R) or (I-S) are selected from C₁₋₄alkyleneNR^{12a}R^{13a}, NR^{12a}R^{13a}, C₃₋₇heterocycloalkyl, and C₁₋₄alkyleneC₃₋₇heterocycloalkyl, the latter two groups being optionally substituted with one to four of R¹³ as defined above for Formula (I). In an embodiment, one R¹¹ in the of Formula (I-R) or (I-S) is C₁₋₂alkyleneNR^{12a}R^{13a} as defined above for Formula (I).

In an embodiment, the compound of Formula (I) is a compound of Formula (I-T) or (I-U) or a pharmaceutically acceptable salt, solvate, and/or prodrug thereof:

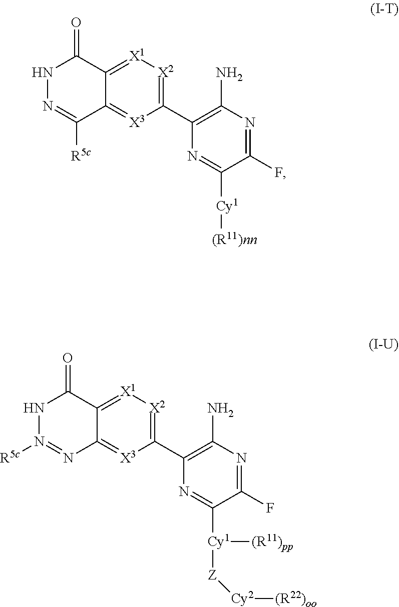

wherein R^{5c}, X¹, X², X³, Z, Cy¹ and Cy² are as defined in Formula (I) above;
R¹¹ is selected from C₁₋₄alkyleneNR^{12a}R^{13a}, NR^{12a}R^{13a}, NR^{13a}COR¹², SO₂NR¹²R¹³, NR^{13a}C₁₋₄alkyleneNR¹²R¹³, C₃₋₇heterocycloalkyl, and C₁₋₄alkyleneC₃₋₇heterocycloalkyl, the latter four groups being optionally substituted with one to four of R¹⁵
R¹², R¹², R¹³, R^{13a} and R¹⁵ are as defined in Formula (I) above;
nn and pp are integers independently selected from 1 to 4, and
oo is an integer selected from 0 to 5.

In an embodiment, nn is 1 or 2, suitably 1. In an embodiment, oo is 1 or 2, suitably 1. In an embodiment, one R¹¹ in the of Formula (I-T) or (I-U) is selected from C₁₋₄alkyleneNR^{12a}R^{13a}, NR^{12a}R^{13a}, NR^{13a}COR¹², NR^{13a}C₁₋₄alkyleneNR¹²R¹³, C₃₋₇heterocycloalkyl, and C₁₋₄alkyleneC₃₋₇heterocycloalkyl, the latter two groups being optionally substituted with one to four of R¹³ as defined above for Formula (I). In an embodiment, one or two R¹¹ in the of Formula (I-T) or (I-U) are selected from C₁₋₄alkyleneNR^{12a}R^{13a}, NR^{12a}R^{13a}, C₃₋₇heterocycloalkyl, and C₁₋₄alkyleneC₃₋₇heterocycloalkyl, the latter two groups being optionally substituted with one to four of R¹⁵ as defined above for Formula (I). In an embodiment, one R¹¹ in the of Formula (I-T) or (I-U) is C₁₋₄alkyleneNR^{12a}R^{13a} as defined above for Formula (I).

In an embodiment, the compound of Formula (I) is selected from the compounds listed in Table 1.

TABLE 1

| Compound | | |
|---|---|---|
| ID # | IUPAC NAME | Structures |
| I-1 | 6-(3-amino-5-fluoro-6-(4-(4- isopropylpiperazin-1- yl)phenyl)pyrazin-2-yl)-3,4- dihydroisoquinolin-1(2H)-one | 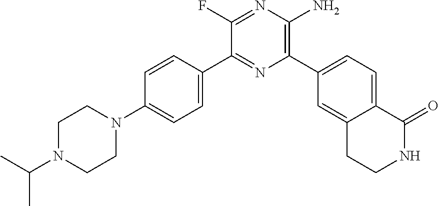 [SMILES: CC(C)N1CCN(c2ccc(-c3nc(-c4ccc5c(c4)CCNC5=O)c(N)nc3F)cc2)CC1] |
| I-2 | 6-(3-amino-5-fluoro-6-(4-(4- isopropylpiperazin-1- yl)phenyl)pyrazin-2-yl)-7-fluoro- 3,4-dihydroisoquinolin-1(2H)- one | 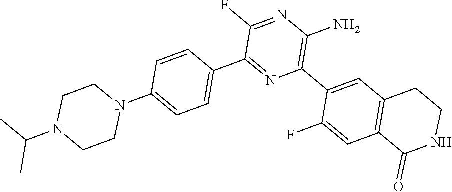 [SMILES: CC(C)N1CCN(c2ccc(-c3nc(-c4cc5c(cc4F)C(=O)NCC5)c(N)nc3F)cc2)CC1] |
| I-3 | 6-(3-amino-5-fluoro-6-(4- ((1R,5S)-3-methyl-3- azabicyclo[3.1.0]hexan-1- yl)phenyl)pyrazin-2-yl)-7-fluoro 3,4-dihydroisoquinolin-1(2H)- one | 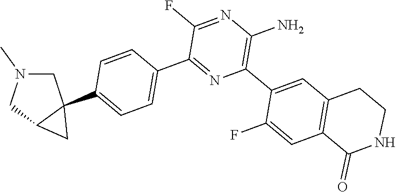 [SMILES: CN1C[C@H]2C[C@@]2(c2ccc(-c3nc(-c4cc5c(cc4F)C(=O)NCC5)c(N)nc3F)cc2)C1] |
| I-4 | 6-(3-amino-5-fluoro-6-(4- ((1R,5S)-3-isopropyl-3- azabicyclo[3.1.0]hexan-1- yl)phenyl)pyrazin-2-yl)-7-fluoro- 3,4-dihydroisoquinolin-1(2H)- one | 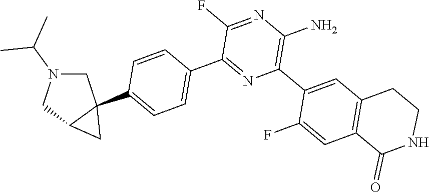 [SMILES: CC(C)N1C[C@H]2C[C@@]2(c2ccc(-c3nc(-c4cc5c(cc4F)C(=O)NCC5)c(N)nc3F)cc2)C1] |
| I-5 | 6-(3-amino-5-fluoro-6-(4- ((1S,5R)-3-methyl-3- azabicyclo[3.1.0]hexan-1- N. 2 yl)phenyl)pyrazin-2-yl)-7-fluoro- 3,4-dihydroisoquinolin-1(2H)- one | 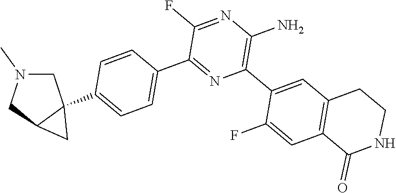 [SMILES: CN1C[C@@H]2C[C@]2(c2ccc(-c3nc(-c4cc5c(cc4F)C(=O)NCC5)c(N)nc3F)cc2)C1] |
| I-6 | 6-(3-amino-5-fluoro-6-(4- ((1S,5R)-3-isopropyl-3- azabicyclo[3.1.0]hexan-1- yl)phenyl)pyrazin-2-yl)-7-fluoro- 3,4-dihydroisoquinolin-1(2H)- one | 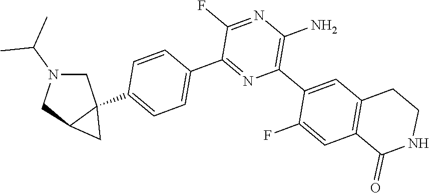 [SMILES: CC(C)N1C[C@@H]2C[C@]2(c2ccc(-c3nc(-c4cc5c(cc4F)C(=O)NCC5)c(N)nc3F)cc2)C1] |
| I-7 | 6-(3-amino-5-chloro-6-(4-(4- isopropylpiperazin-1- yl)phenyl)pyrazin-2-yl)-3,4- dihydroisoquinolin-1(2H)-one | 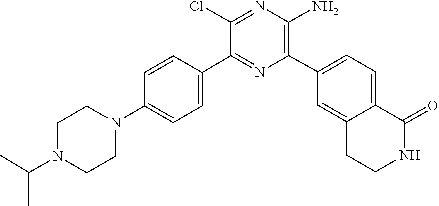 [SMILES: CC(C)N1CCN(c2ccc(-c3nc(-c4ccc5c(c4)CCNC5=O)c(N)nc3Cl)cc2)CC1] |
| I-8 | 6-(3-amino-5-chloro-6-(4-(4- isopropylpiperazin-1- yl)phenyl)pyrazin-2-yl)-7-fluoro- 3,4-dihydroisoquinolin-1(2H)- one | 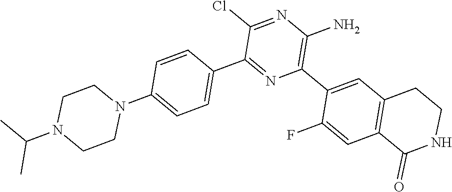 [SMILES: CC(C)N1CCN(c2ccc(-c3nc(-c4cc5c(cc4F)C(=O)NCC5)c(N)nc3Cl)cc2)CC1] |
| I-9 | 6-(3-amino-5-fluoro-6-(4-(1- isopropyl-1,2,3,6- tetrahydropyridin-4- yl)phenyl)pyrazin-2-yl)-7-fluoro- 3,4-dihydroisoquinolin-1(2H)- one | 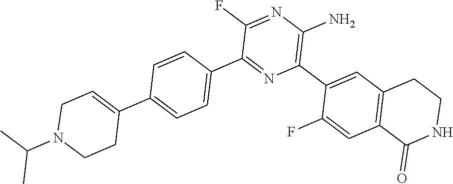 [SMILES: CC(C)N1CC=C(c2ccc(-c3nc(-c4cc5c(cc4F)C(=O)NCC5)c(N)nc3F)cc2)CC1] |
| I-10 | 6-(3-amino-5-fluoro-6-(4-(1- isopropylpiperidin-4- yl)phenyl)pyrazin-2-yl)-8-fluoro- 3,4-dihydroisoquinolin-1(2H)- one | 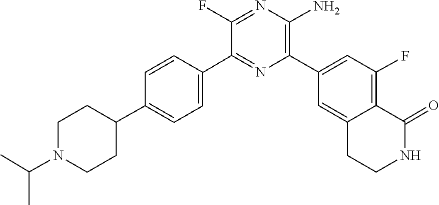 [SMILES: CC(C)N1CCC(c2ccc(-c3nc(-c4cc(F)c5c(c4)CCNC5=O)c(N)nc3F)cc2)CC1] |
| I-11 | 6-(3-amino-5-fluoro-6-(4-(4- isopropylpiperazin-1- yl)phenyl)pyrazin-2-yl)-3- N ((methylamino)methyl)-3,4- dihydroisoquinolin-1(2H)-one | 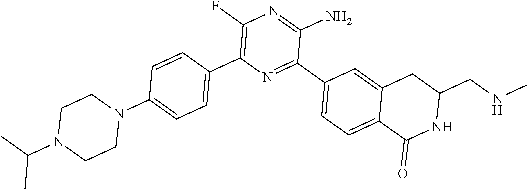 [SMILES: CNCC1Cc2cc(-c3nc(-c4ccc(N5CCN(C(C)C)CC5)cc4)c(F)nc3N)ccc2C(=O)N1] |
| I-12 | 6-(3-amino-5-fluoro-6-(4- morpholinophenyl)pyrazin-2- yl)-3-((methylamino)methyl)- 3,4-dihydroisoquinolin-1(2H)- one | 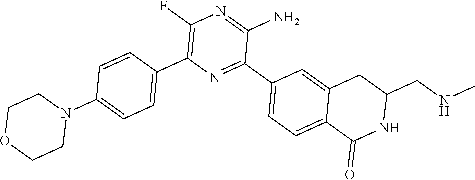 [SMILES: CNCC1Cc2cc(-c3nc(-c4ccc(N5CCOCC5)cc4)c(F)nc3N)ccc2C(=O)N1] |
| I-113 | 6-(3-amino-6-(4-(1- (cyclopropylmethyl)piperidin-4- yl)phenyl)-5-fluoropyrazin-2-yl)- 8-fluoro-3,4-dihydroisoquinolin- 1(2H)-one formate | 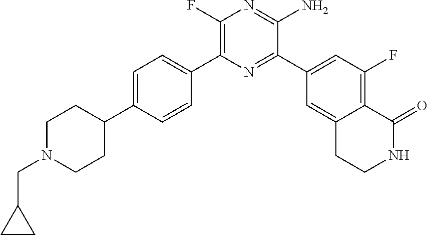 [SMILES: Nc1nc(F)c(-c2ccc(C3CCN(CC4CC4)CC3)cc2)nc1-c1cc(F)c2c(c1)CCNC2=O] |
| I-14 | 6-(3-amino-5-fluoro-6-(4-(4- isopropylpiperazin-1- yl)phenyl)pyrazin-2-yl)-8-fluoro- 3,4-dihydroisoquinolin-1(2H)- one | 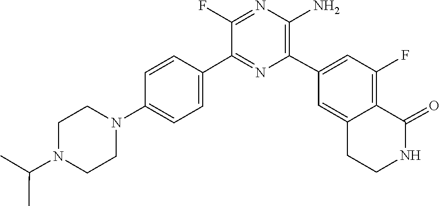 [SMILES: CC(C)N1CCN(c2ccc(-c3nc(-c4cc(F)c5c(c4)CCNC5=O)c(N)nc3F)cc2)CC1] |
| I-15 | 6-(3-amino-5-fluoro-6-(4- ((1R,5S)-3-isopropyl-3- azabicyclo[3.1.0]hexan-1- yl)phenyl)pyrazin-2-yl)-8-fluoro- 3,4-dihydroisoquinolin-1(2H)- one formate | 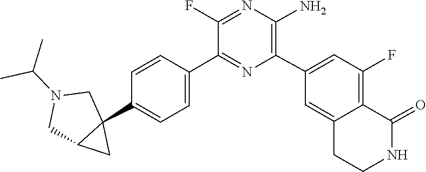 [SMILES: CC(C)N1C[C@H]2C[C@@]2(c2ccc(-c3nc(-c4cc(F)c5c(c4)CCNC5=O)c(N)nc3F)cc2)C1] |
| I-16 | 6-(3-amino-6-(4-(4- (cyclopropylmethyl)piperazin-1- yl)phenyl)-5-fluoropyrazin-2-yl)- 8-fluoro-3,4-dihydroisoquinolin- 1(2H)-one | 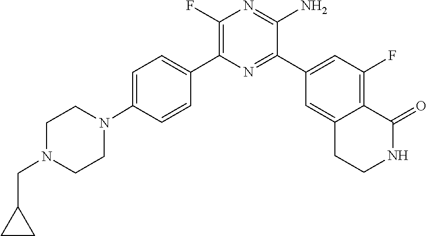 [SMILES: Nc1nc(F)c(-c2ccc(N3CCN(CC4CC4)CC3)cc2)nc1-c1cc(F)c2c(c1)CCNC2=O] |
| I-17 | 6-(3-amino-5-fluoro-6-(4- morpholinophenyl)pyrazin-2- yl)-3,4-dihydroisoquinolin- 1(2H)-one (I-17) | 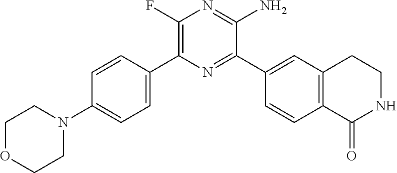 [SMILES: Nc1nc(F)c(-c2ccc(N3CCOCC3)cc2)nc1-c1ccc2c(c1)CCNC2=O] |
| I-18 | (S)-6-(3-amino-5-fluoro-6-(3- (pyrrolidin-2-yl)-4-(tetrahydro- 2H-pyran-4-yl)phenyl)pyrazin- 2-yl)-3,4-dihydroisoquinolin- 1(2H)-one | 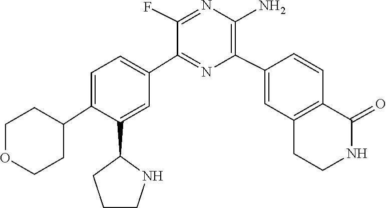 [SMILES: Nc1nc(F)c(-c2ccc(C3CCOCC3)c([C@@H]3CCCN3)c2)nc1-c1ccc2c(c1)CCNC2=O] |
| I-19 | (S)-6-(3-amino-5-fluoro-6-(3- (pyrrolidin-2-yl)-4-(tetrahydro- 2H-pyran-4-yl)phenyl)pyrazin- 2-yl)-7-fluoro-3,4- dihydroisoquinolin-1(2H)-one | 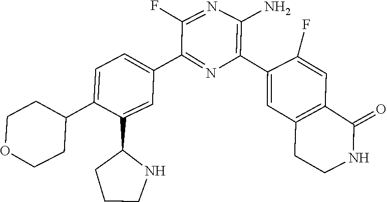 [SMILES: Nc1nc(F)c(-c2ccc(C3CCOCC3)c([C@@H]3CCCN3)c2)nc1-c1cc2c(cc1F)C(=O)NCC2] |
| I-20 | 6-(3-amino-5-fluoro-6-(4-(4- isopropylpiperazin-1- yl)phenyl)pyrazin-2- yl)isoquinolin-1(2H)-one | 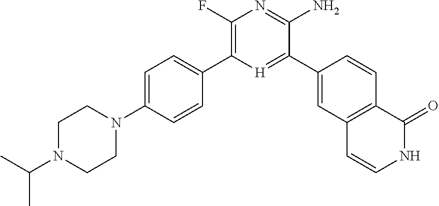 |
| I-21 | 6-(3-amino-5-fluoro-6-(4-(4- isopropylpiperazin-1- yl)phenyl)pyrazin-2-yl)-8- fluoroisoquinolin-1(2H)-one | 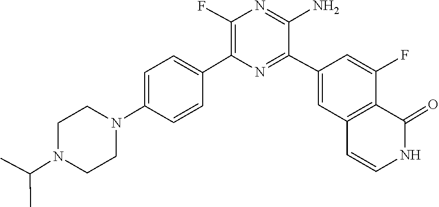 [SMILES: CC(C)N1CCN(c2ccc(-c3nc(-c4cc(F)c5c(=O)[nH]ccc5c4)c(N)nc3F)cc2)CC1] |
| I-22 | 6-(3-amino-5-fluoro-6-(4-(4- isopropylpiperazin-1- yl)phenyl)pyrazin-2-yl)-7- fluoroisoquinolin-1(2H)-one | 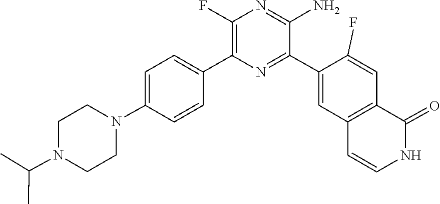 [SMILES: CC(C)N1CCN(c2ccc(-c3nc(-c4cc5cc[nH]c(=O)c5cc4F)c(N)nc3F)cc2)CC1] |
| I-23 | 6-(3-amino-6-(4-(4- (cyclopropylmethyl)piperazin-1- yl)phenyl)-5-fluoropyrazin-2- yl)isoquinolin-1(2H)-one | 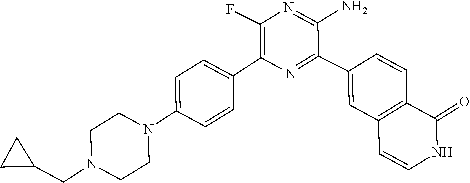 [SMILES: Nc1nc(F)c(-c2ccc(N3CCN(CC4CC4)CC3)cc2)nc1-c1ccc2c(=O)[nH]ccc2c1] |
| I-24 | 6-(3-amino-6-(4-(4- (cyclopropylmethyl)piperazin-1- yl)phenyl)-5-fluoropyrazin-2-yl)- 8-fluoroisoquinolin-1(2H)-one | 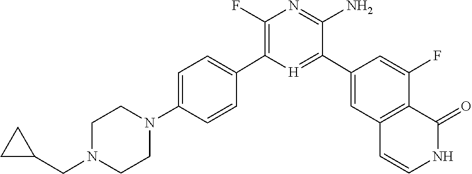 |
| I-25 | 6-(3-amino-6-(4-(4- (cyclopropylmethyl)piperazin-1- yl)phenyl)-5-fluoropyrazin-2-yl)- 7-fluoroisoquinolin-1(2H)-one | 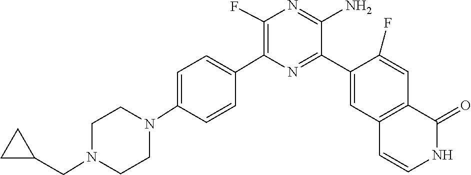 [SMILES: Nc1nc(F)c(-c2ccc(N3CCN(CC4CC4)CC3)cc2)nc1-c1cc2cc[nH]c(=O)c2cc1F] |
| I-26 | 6-(3-amino-6-(4-(4-(2- cyclopropylethyl)piperazin-1- yl)phenyl)-5-fluoropyrazin-2-yl)- 8-fluoro-3,4-dihydroisoquinolin- 1(2H)-one | 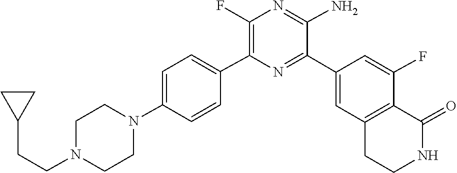 [SMILES: Nc1nc(F)c(-c2ccc(N3CCN(CCC4CC4)CC3)cc2)nc1-c1cc(F)c2c(c1)CCNC2=O] |
| I-27 | 6-(3-amino-6-(4-(4-(2- cyclopropylethyl)piperazin-1- yl)phenyl)-5-fluoropyrazin-2-yl)- 8-fluoroisoquinolin-1(2H)-one | 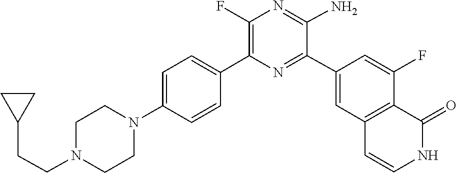 [SMILES: Nc1nc(F)c(-c2ccc(N3CCN(CCC4CC4)CC3)cc2)nc1-c1cc(F)c2c(=O)[nH]ccc2c1] |
| I-28 | 6-(3-amino-6-(4-(4-(2- cyclopropylethyl)piperazin-1- yl)phenyl)-5-fluoropyrazin-2-yl)- 7-fluoroisoquinolin-1(2H)-one | 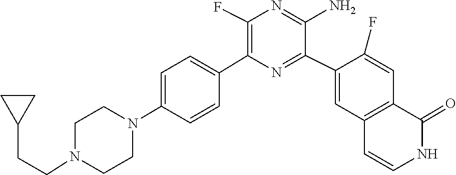 [SMILES: Nc1nc(F)c(-c2ccc(N3CCN(CCC4CC4)CC3)cc2)nc1-c1cc2cc[nH]c(=O)c2cc1F] |
| I-29 | (R)-6-(3-amino-6-(4-(4-(1- cyclopropylethyl)piperazin-1- yl)phenyl)-5-fluoropyrazin-2-yl)- 8-fluoro-3,4-dihydroisoquinolin- 1(2H)-one, enantiomer 1 | 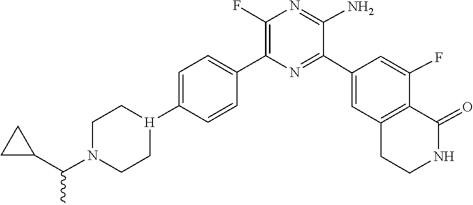 |
| I-30 | (S)-6-(3-amino-6-(4-(4-(1- cyclopropylethyl)piperazin-1- yl)phenyl)-5-fluoropyrazin-2-yl)- 8-fluoro-3,4-dihydroisoquinolin- 1(2H)-one, enantiomer 2 | 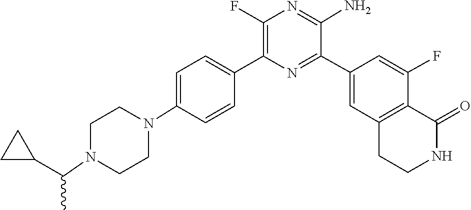 [SMILES: CC(C1CC1)N1CCN(c2ccc(-c3nc(-c4cc(F)c5c(c4)CCNC5=O)c(N)nc3F)cc2)CC1] |
| I-31 | 6-(3-amino-6-(4-(4- ethylpiperazin-1-yl)phenyl)-5- fluoropyrazin-2-yl)-8- fluoroisoquinolin-1(2H)-one | 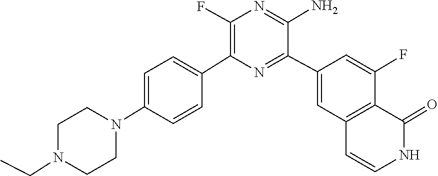 [SMILES: CCN1CCN(c2ccc(-c3nc(-c4cc(F)c5c(=O)[nH]ccc5c4)c(N)nc3F)cc2)CC1] |
| I-32 | 6-(3-amino-6-(4-(4- ethylpiperazin-1-yl)phenyl)-5- fluoropyrazin-2-yl)isoquinolin- 1(2H)-one | 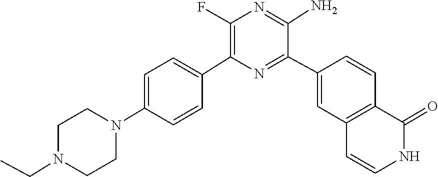 [SMILES: CCN1CCN(c2ccc(-c3nc(-c4ccc5c(=O)[nH]ccc5c4)c(N)nc3F)cc2)CC1] |
| I-33 | 6-(3-amino-5-fluoro-6-(4- morpholinophenyl)pyrazin-2- yl)-3,4-dihydroisoquinolin- 1(2H)-one | 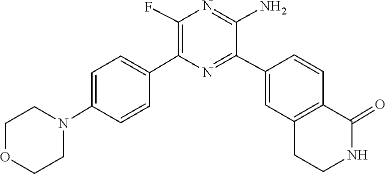 [SMILES: Nc1nc(F)c(-c2ccc(N3CCOCC3)cc2)nc1-c1ccc2c(c1)CCNC2=O] |
| I-34 | 6-(3-amino-5-fluoro-6-(4- morpholinophenyl)pyrazin-2- yl)-7-fluoro-3,4- dihydroisoquinolin-1(2H)-one | 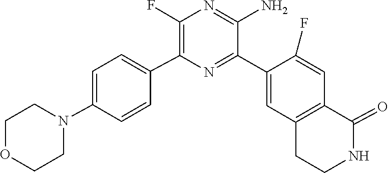 [SMILES: Nc1nc(F)c(-c2ccc(N3CCOCC3)cc2)nc1-c1cc2c(cc1F)C(=O)NCC2] |
| I-35 | 6-(3-amino-5-fluoro-6-(4- morpholinophenyl)pyrazin-2- yl)-8-fluoro-3,4- dihydroisoquinolin-1(2H)-one | 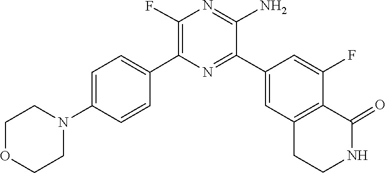 [SMILES: Nc1nc(F)c(-c2ccc(N3CCOCC3)cc2)nc1-c1cc(F)c2c(c1)CCNC2=O] |
| I-36 | 6-(3-amino-5-fluoro-6-(4- morpholinophenyl)pyrazin-2- yl)-8-fluoroisoquinolin-1(2H)- one | 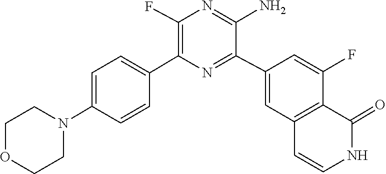 [SMILES: Nc1nc(F)c(-c2ccc(N3CCOCC3)cc2)nc1-c1cc(F)c2c(=O)[nH]ccc2c1] |
| I-37 | 7-(3-amino-5-fluoro-6-(4-(4- isopropylpiperazin-1- yl)phenyl)pyrazin-2- yl)quinazolin-4(3H)-one | 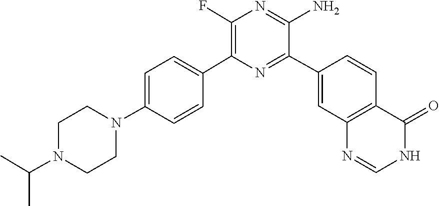 [SMILES: CC(C)N1CCN(c2ccc(-c3nc(-c4ccc5c(=O)[nH]cnc5c4)c(N)nc3F)cc2)CC1] |
| I-38 | 6-(3-amino-5-fluoro-6-(4-(4- isopropylpiperazin-1- yl)phenyl)pyrazin-2- yl)phthalazin-1(2H)-one | 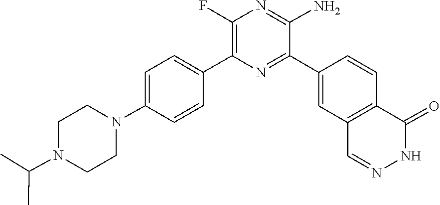 [SMILES: CC(C)N1CCN(c2ccc(-c3nc(-c4ccc5c(=O)[nH]ncc5c4)c(N)nc3F)cc2)CC1] |
| I-39 | 7-(3-amino-5-fluoro-6-(4-(4- isopropylpiperazin-1- yl)phenyl)pyrazin-2-yl)-2- methylquinazolin-4(3H)-one | 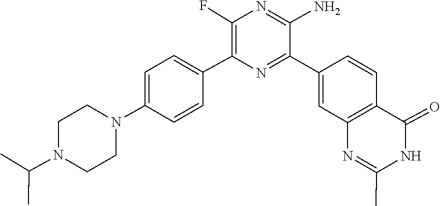 [SMILES: Cc1nc2cc(-c3nc(-c4ccc(N5CCN(C(C)C)CC5)cc4)c(F)nc3N)ccc2c(=O)[nH]1] |
| I-40 | 7-(3-amino-5-fluoro-6-(4-(4- isopropylpiperazin-1- yl)phenyl)pyrazin-2-yl)-5-fluoro- 2-methylquinazolin-4(3H)-one | 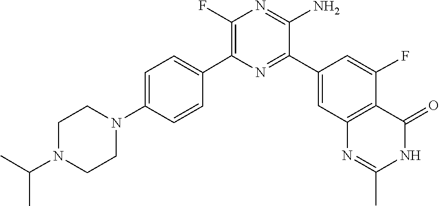 [SMILES: Cc1nc2cc(-c3nc(-c4ccc(N5CCN(C(C)C)CC5)cc4)c(F)nc3N)cc(F)c2c(=O)[nH]1] |
| I-41 | 6-(3-amino-5-fluoro-6-(4-(4- methylpiperazin-1- yl)phenyl)pyrazin-2-yl)-3,4- dihydroisoquinolin-1(2H)-one | 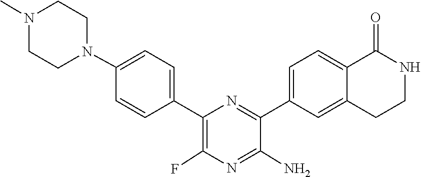 [SMILES: CN1CCN(c2ccc(-c3nc(-c4ccc5c(c4)CCNC5=O)c(N)nc3F)cc2)CC1] |
| I-42 | 6-(3-amino-6-(3- ((dimethylamino)methyl)-4- (tetrahydro-2H-pyran-4- yl)phenyl)-5-fluoropyrazin-2-yl)- 4-fluoroisoquinolin-1(2H)-one | 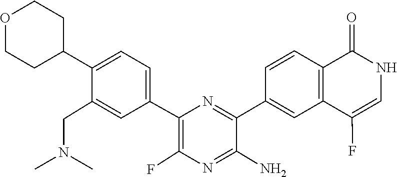 [SMILES: CN(C)Cc1cc(-c2nc(-c3ccc4c(=O)[nH]cc(F)c4c3)c(N)nc2F)ccc1C1CCOCC1] |
| I-43 | 6-(3-amino-5-fluoro-6-(4-(4- isopropylpiperazin-1- yl)phenyl)pyrazin-2-yl)-4- chloro-3-methylisoquinolin- 1(2H)-one | 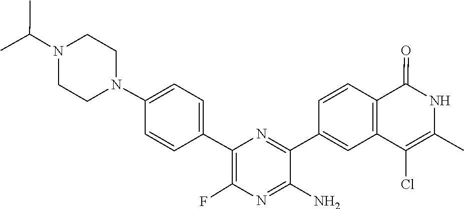 [SMILES: Cc1[nH]c(=O)c2ccc(-c3nc(-c4ccc(N5CCN(C(C)C)CC5)cc4)c(F)nc3N)cc2c1Cl] |
| I-44 | 6-(3-amino-5-fluoro-6-(4-(4- (oxetan-3-yl)piperazin-1- yl)phenyl)pyrazin-2-yl)-8-fluoro- 3,4-dihydroisoquinolin-1(2H)- one | 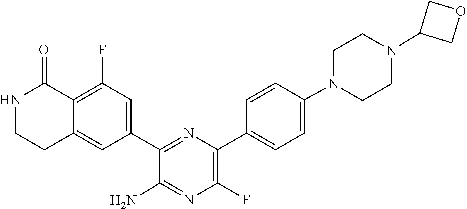 [SMILES: Nc1nc(F)c(-c2ccc(N3CCN(C4COC4)CC3)cc2)nc1-c1cc(F)c2c(c1)CCNC2=O] |
| I-45 | 6-(3-amino-5-fluoro-6-(4-(4- (oxetan-3-yl)piperazin-1- yl)phenyl)pyrazin-2-yl)-7-fluoro- 3,4-dihydroisoquinolin-1(2H)- one | 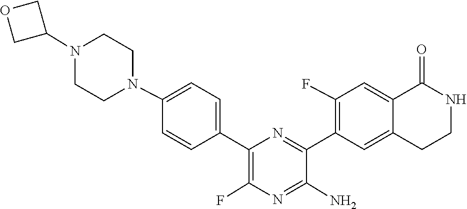 [SMILES: Nc1nc(F)c(-c2ccc(N3CCN(C4COC4)CC3)cc2)nc1-c1cc2c(cc1F)C(=O)NCC2] |
| I-46 | 6-(3-amino-5-fluoro-6-(4-(4- (oxetan-3-yl)piperazin-1- yl)phenyl)pyrazin-2-yl)-3,4- dihydroisoquinolin-1(2H)-one | 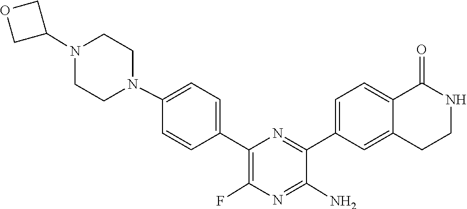 [SMILES: Nc1nc(F)c(-c2ccc(N3CCN(C4COC4)CC3)cc2)nc1-c1ccc2c(c1)CCNC2=O] |
| I-47 | 6-(3-amino-6-(4-(4- cyclobutylpiperazin-1- yl)phenyl)-5-fluoropyrazin-2-yl)- 8-fluoro-3,4-dihydroisoquinolin- 1(2H)-one | 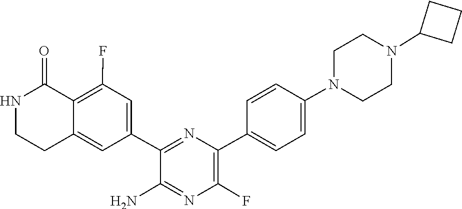 [SMILES: Nc1nc(F)c(-c2ccc(N3CCN(C4CCC4)CC3)cc2)nc1-c1cc(F)c2c(c1)CCNC2=O] |
| I-48 | 6-(3-amino-6-(4-(4- cyclobutylpiperazin-1- yl)phenyl)-5-fluoropyrazin-2-yl)- 7-fluoro-3,4-dihydroisoquinolin- 1(2H)-one | 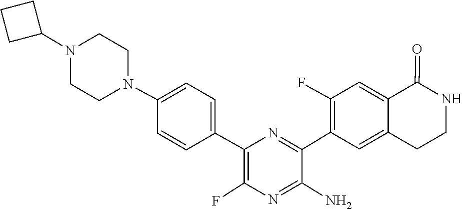 [SMILES: Nc1nc(F)c(-c2ccc(N3CCN(C4CCC4)CC3)cc2)nc1-c1cc2c(cc1F)C(=O)NCC2] |
| I-49 | 6-(3-amino-6-(4-(4- cyclobutylpiperazin-1- yl)phenyl)-5-fluoropyrazin-2-yl)- 3,4-dihydroisoquinolin-1(2H)- one | 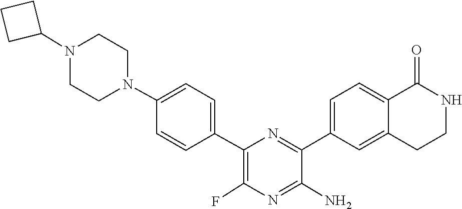 [SMILES: Nc1nc(F)c(-c2ccc(N3CCN(C4CCC4)CC3)cc2)nc1-c1ccc2c(c1)CCNC2=O] |
| I-50 | (R)-6-(3-amino-5-fluoro-6-(4- (4-isopropyl-2-methylpiperazin- 1-yl)phenyl)pyrazin-2-yl)-3,4- dihydroisoquinolin-1(2H)-one | 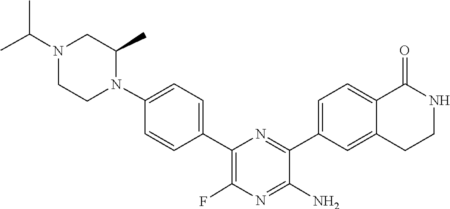 [SMILES: CC(C)N1CCN(c2ccc(-c3nc(-c4ccc5c(c4)CCNC5=O)c(N)nc3F)cc2)[C@H](C)C1] |
| I-51 | (R)-6-(3-amino-5-fluoro-6-(4- (4-isopropyl-2-methylpiperazin- 1-yl)phenyl)pyrazin-2-yl)-7- fluoro-3,4-dihydroisoquinolin- 1(2H)-one | 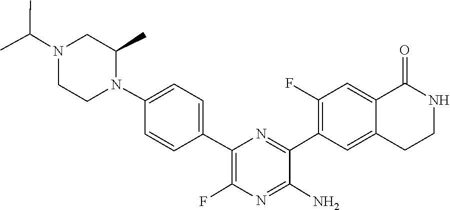 [SMILES: CC(C)N1CCN(c2ccc(-c3nc(-c4cc5c(cc4F)C(=O)NCC5)c(N)nc3F)cc2)[C@H](C)C1] |
| I-52 | (R)-6-(3-amino-6-(4-(2,4- dimethylpiperazin-1-yl)phenyl)- 5-fluoropyrazin-2-yl)-3,4- dihydroisoquinolin-1(2H)-one | 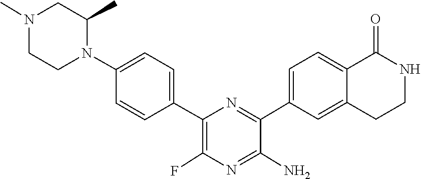 [SMILES: C[C@@H]1CN(C)CCN1c1ccc(-c2nc(-c3ccc4c(c3)CCNC4=O)c(N)nc2F)cc1] |
| I-53 | (R)-6-(3-amino-6-(4-(2,4- dimethylpiperazin-1-yl)phenyl)- 5-fluoropyrazin-2-yl)-7-fluoro- 3,4-dihydroisoquinolin-1(2H)- one | 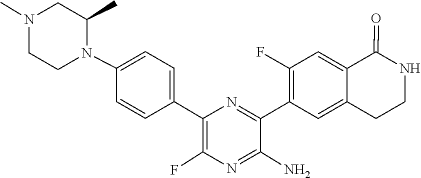 [SMILES: C[C@@H]1CN(C)CCN1c1ccc(-c2nc(-c3cc4c(cc3F)C(=O)NCC4)c(N)nc2F)cc1] |
| I-54 | 6-(3-amino-5-fluoro-6-(4-(4- methylpiperazin-1- yl)phenyl)pyrazin-2-yl)-7-fluoro- 3,4-dihydroisoquinolin-1(2H)- one | 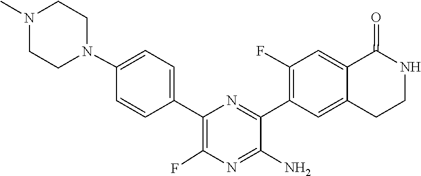 [SMILES: CN1CCN(c2ccc(-c3nc(-c4cc5c(cc4F)C(=O)NCC5)c(N)nc3F)cc2)CC1] |
| I-55 | 6-(3-amino-6-(4-(4- ethylpiperazin-1-yl)phenyl)-5- fluoropyrazin-2-yl)-3,4- dihydroisoquinolin-1(2H)-one | 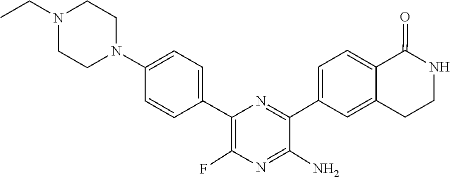 [SMILES: CCN1CCN(c2ccc(-c3nc(-c4ccc5c(c4)CCNC5=O)c(N)nc3F)cc2)CC1] |
| I-56 | 6-(3-amino-6-(4-(4- ethylpiperazin-1-yl)phenyl)-5- fluoropyrazin-2-yl)-7-fluoro-3,4- dihydroisoquinolin-1(2H)-one | 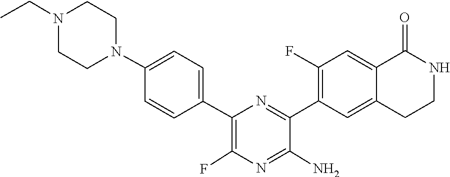 [SMILES: CCN1CCN(c2ccc(-c3nc(-c4cc5c(cc4F)C(=O)NCC5)c(N)nc3F)cc2)CC1] |
| I-57 | 6-(3-amino-5-fluoro-6-(4- ((3R,5S)-3,4,5- trimethylpiperazin-1- yl)phenyl)pyrazin-2-yl)-3,4- dihydroisoquinolin-1(2H)-one | 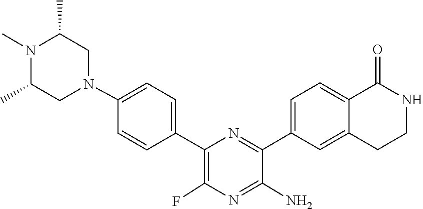 [SMILES: C[C@@H]1CN(c2ccc(-c3nc(-c4ccc5c(c4)CCNC5=O)c(N)nc3F)cc2)C[C@H](C)N1C] |
| I-58 | 6-(3-amino-5-fluoro-6-(4- ((3R,5S)-3,4,5- trimethylpiperazin-1- yl)phenyl)pyrazin-2-yl)-7-fluoro- 3,4-dihydroisoquinolin-1(2H)- one | 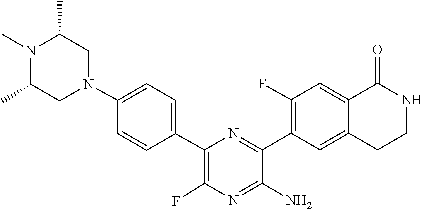 [SMILES: C[C@@H]1CN(c2ccc(-c3nc(-c4cc5c(cc4F)C(=O)NCC5)c(N)nc3F)cc2)C[C@H](C)N1C] |
| I-59 | 6-(3-amino-6-(3- ((dimethylamino)methyl)-4- (tetrahydro-2H-pyran-4- yl)phenyl)-5-fluoropyrazin-2-yl)- 3,4-dihydroisoquinolin-1(2H)- one | 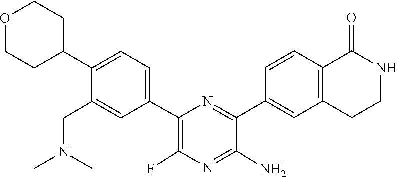 [SMILES: CN(C)Cc1cc(-c2nc(-c3ccc4c(c3)CCNC4=O)c(N)nc2F)ccc1C1CCOCC1] |
| I-60 | 6-(3-amino-6-(3- ((dimethylamino)methyl)-4- (tetrahydro-2H-pyran-4- yl)phenyl)-5-fluoropyrazin-2-yl)- 7-fluoro-3,4-dihydroisoquinolin- 1(2H)-one | 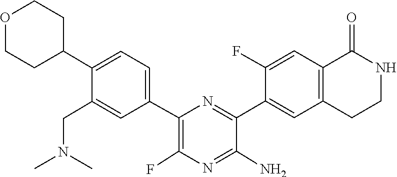 [SMILES: CN(C)Cc1cc(-c2nc(-c3cc4c(cc3F)C(=O)NCC4)c(N)nc2F)ccc1C1CCOCC1] |
| I-61 | 6-(3-amino-5-fluoro-6-(4-(2- methyl-2,7- diazaspiro[3.5]nonan-7- yl)phenyl)pyrazin-2-yl)-7-fluoro- 3,4-dihydroisoquinolin-1(2H)- one | 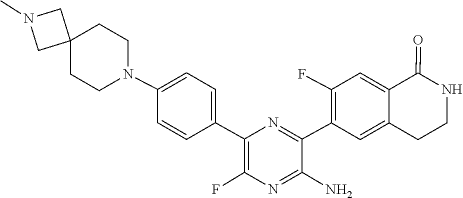 [SMILES: CN1CC2(CCN(c3ccc(-c4nc(-c5cc6c(cc5F)C(=O)NCC6)c(N)nc4F)cc3)CC2)C1] |
| I-62 | 6-(3-amino-5-fluoro-6-(4-(4- isopropylpiperazin-1- yl)phenyl)pyrazin-2-yl)-4- methylisoquinolin-1(2H)-one | 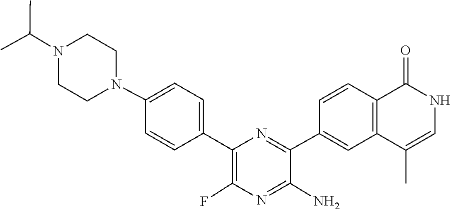 [SMILES: Cc1c[nH]c(=O)c2ccc(-c3nc(-c4ccc(N5CCN(C(C)C)CC5)cc4)c(F)nc3N)cc12] |
| I-63 | 7-(3-amino-5-fluoro-6-(4-(4- isopropylpiperazin-1- yl)phenyl)pyrazin-2-yl)-6-fluoro- 2-methylquinazolin-4(3H)-one | 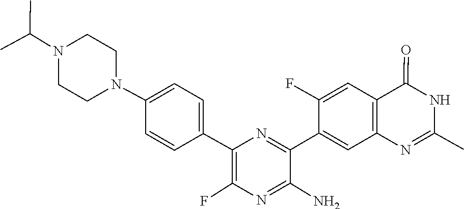 [SMILES: Cc1nc2cc(-c3nc(-c4ccc(N5CCN(C(C)C)CC5)cc4)c(F)nc3N)c(F)cc2c(=O)[nH]1] |
| I-64 | 6-(3-amino-6-(3- ((dimethylamino)methyl)-4- morpholinophenyl)-5- fluoropyrazin-2-yl)-3,4- dihydroisoquinolin-1(2H)-one | 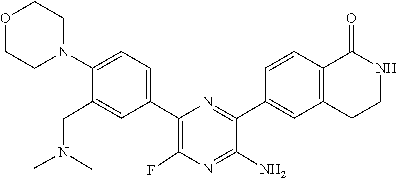 [SMILES: CN(C)Cc1cc(-c2nc(-c3ccc4c(c3)CCNC4=O)c(N)nc2F)ccc1N1CCOCC1] |
| I-65 | 6-(3-amino-6-(3- ((dimethylamino)methyl)-4- morpholinophenyl)-5- fluoropyrazin-2-yl)-7-fluoro-3,4- dihydroisoquinolin-1(2H)-one | 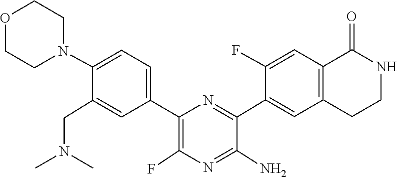 [SMILES: CN(C)Cc1cc(-c2nc(-c3cc4c(cc3F)C(=O)NCC4)c(N)nc2F)ccc1N1CCOCC1] |
| I-66 | 6-(3-amino-5-fluoro-6-(4-(4- isopropylpiperazin-1- yl)phenyl)pyrazin-2-yl)-4- fluoroisoquinolin-1(2H)-one | 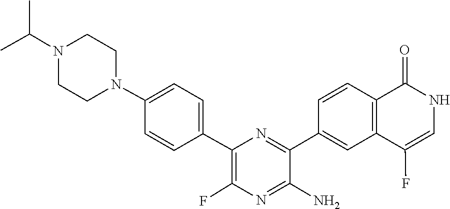 [SMILES: CC(C)N1CCN(c2ccc(-c3nc(-c4ccc5c(=O)[nH]cc(F)c5c4)c(N)nc3F)cc2)CC1] |
| I-67 | 6-(3-amino-5-fluoro-6-(4-(4- isopropylpiperazin-1- yl)phenyl)pyrazin-2-yl)-8-fluoro- 3-methylisoquinolin-1(2H)-one | 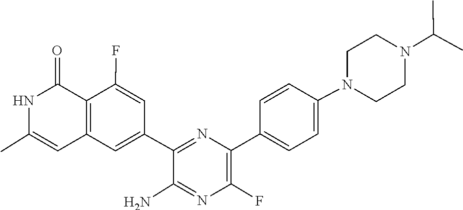 [SMILES: Cc1cc2cc(-c3nc(-c4ccc(N5CCN(C(C)C)CC5)cc4)c(F)nc3N)cc(F)c2c(=O)[nH]1] |
| I-68 | 6-(3-amino-6-(4-(4-(4,4- difluorobutyl)piperazin-1- yl)phenyl)-5-fluoropyrazin-2-yl)- 3,4-dihydroisoquinolin-1(2H)- one | 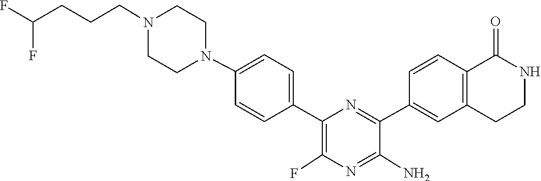 [SMILES: Nc1nc(F)c(-c2ccc(N3CCN(CCCC(F)F)CC3)cc2)nc1-c1ccc2c(c1)CCNC2=O] |
| I-69 | 6-(3-amino-6-(4-(4-(4,4- difluorobutyl)piperazin-1- yl)phenyl)-5-fluoropyrazin-2-yl)- 7-fluoro-3,4-dihydroisoquinolin- 1(2H)-one | 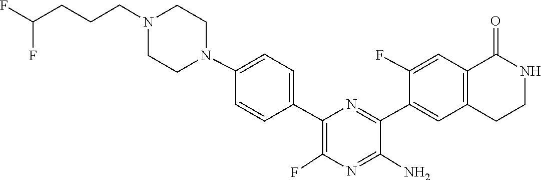 [SMILES: Nc1nc(F)c(-c2ccc(N3CCN(CCCC(F)F)CC3)cc2)nc1-c1cc2c(cc1F)C(=O)NCC2] |
| I-70 | 6-(3-amino-6-(4-(4-(4,4- difluorobutyl)piperazin-1- yl)phenyl)-5-fluoropyrazin-2- yl)isoquinolin-1(2H)-one | 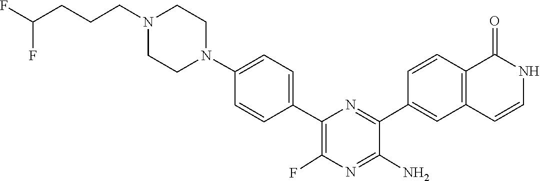 [SMILES: Nc1nc(F)c(-c2ccc(N3CCN(CCCC(F)F)CC3)cc2)nc1-c1ccc2c(=O)[nH]ccc2c1] |
| I-71 | 6-(3-amino-5-fluoro-6-(4-(4- isopropylpiperazin-1- yl)phenyl)pyrazin-2-yl)-4-fluoro- 3-methylisoquinolin-1(2H)-one | 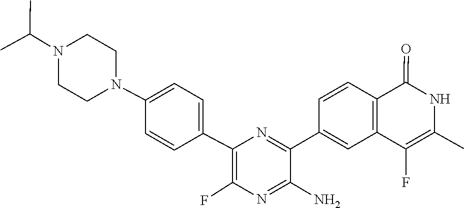 [SMILES: Cc1[nH]c(=O)c2ccc(-c3nc(-c4ccc(N5CCN(C(C)C)CC5)cc4)c(F)nc3N)cc2c1F] |
| I-72 | 6-(3-amino-6-(4-(4- (cyclopropylmethyl)piperazin-1- yl)phenyl)-5-fluoropyrazin-2-yl)- 4-methylisoquinolin-1(2H)-one | 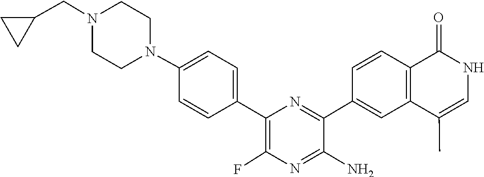 [SMILES: Cc1c[nH]c(=O)c2ccc(-c3nc(-c4ccc(N5CCN(CC6CC6)CC5)cc4)c(F)nc3N)cc12] |
| I-73 | 6-(3-amino-6-(4-(4-(4,4- difluorobutyl)piperazin-1- yl)phenyl)-5-fluoropyrazin-2-yl)- 4-methylisoquinolin-1(2H)-one | 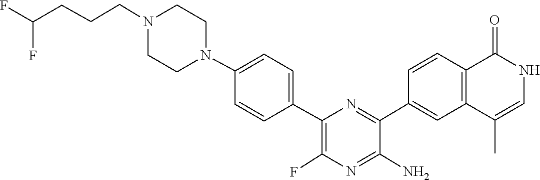 [SMILES: Cc1c[nH]c(=O)c2ccc(-c3nc(-c4ccc(N5CCN(CCCC(F)F)CC5)cc4)c(F)nc3N)cc12] |
| I-74 | 7-(3-amino-5-fluoro-6-(4- ((1S,5R)-3-methyl-3- azabicyclo[3.1.0]hexan-1- yl)phenyl)pyrazin-2-yl)-2- methylquinazolin-4(3H)-one | 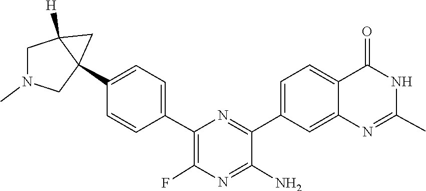 |
| I-75 | 6-(3-amino-5-fluoro-6-(4- ((1S,5R)-3-methyl-3- azabicyclo[3.1.0]hexan-1- yl)phenyl)pyrazin-2-yl)-4- methylisoquinolin-1(2H)-one | 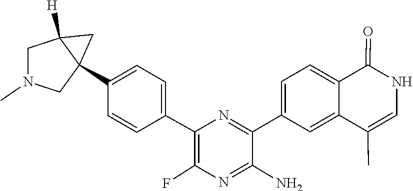 [SMILES: Cc1c[nH]c(=O)c2ccc(-c3nc(-c4ccc([C@]56C[C@H]5CN(C)C6)cc4)c(F)nc3N)cc12] |
| I-76 | 7-(3-amino-6-(4-(4- cyclobutylpiperazin-1- yl)phenyl)-5-fluoropyrazin-2-yl)- 2-methylquinazolin-4(3H)-one | 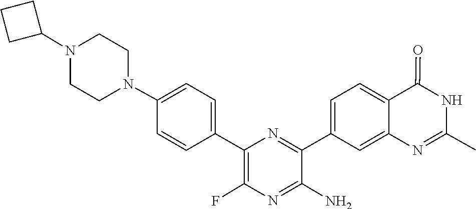 [SMILES: Cc1nc2cc(-c3nc(-c4ccc(N5CCN(C6CCC6)CC5)cc4)c(F)nc3N)ccc2c(=O)[nH]1] |
| I-77 | 7-(3-amino-6-(3- ((dimethylamino)methyl)-4- morpholinophenyl)-5- fluoropyrazin-2-yl)-2- methylquinazolin-4(3H)-one | 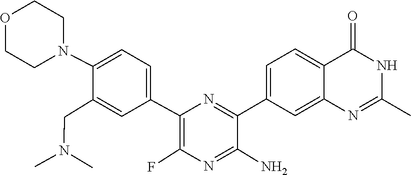 [SMILES: Cc1nc2cc(-c3nc(-c4ccc(N5CCOCC5)c(CN(C)C)c4)c(F)nc3N)ccc2c(=O)[nH]1] |
| I-78 | 6-(3-amino-5-fluoro-6-(4-(4- isopropylpiperazin-1- yl)phenyl)pyrazin-2-yl)-4- chloroisoquinolin-1(2H)-one | 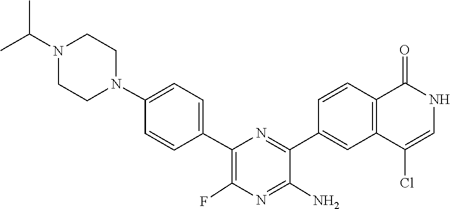 [SMILES: CC(C)N1CCN(c2ccc(-c3nc(-c4ccc5c(=O)[nH]cc(Cl)c5c4)c(N)nc3F)cc2)CC1] |
| I-79 | 7-(3-amino-6-(4-(4- (cyclopropylmethyl)piperazin-1- yl)phenyl)-5-fluoropyrazin-2-yl)- 2-methylquinazolin-4(3H)-one | 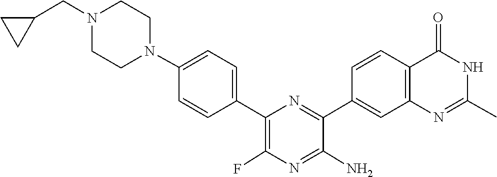 [SMILES: Cc1nc2cc(-c3nc(-c4ccc(N5CCN(CC6CC6)CC5)cc4)c(F)nc3N)ccc2c(=O)[nH]1] |
| I-80 | 6-(3-amino-5-fluoro-6-(4- ((1S,5R)-3-methyl-3- azabicyclo[3.1.0]hexan-1- yl)phenyl)pyrazin-2-yl)-4- fluoroisoquinolin-1(2H)-one | 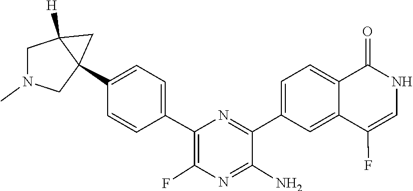 [SMILES: CN1C[C@@H]2C[C@]2(c2ccc(-c3nc(-c4ccc5c(=O)[nH]cc(F)c5c4)c(N)nc3F)cc2)C1] |
| I-81 | 7-(3-amino-6-(4-(4-(4,4- difluorobutyl)piperazin-1- yl)phenyl)-5-fluoropyrazin-2-yl)- 2-methylquinazolin-4(3H)-one | 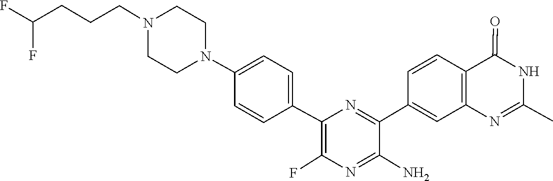 [SMILES: Cc1nc2cc(-c3nc(-c4ccc(N5CCN(CCCC(F)F)CC5)cc4)c(F)nc3N)ccc2c(=O)[nH]1] |
| I-82 | 6-(3-amino-6-(4-(4-(3,3- difluoropropyl)piperazin-1- yl)phenyl)-5-fluoropyrazin-2-yl)- 8-fluoro-3,4-dihydroisoquinolin- 1(2H)-one | 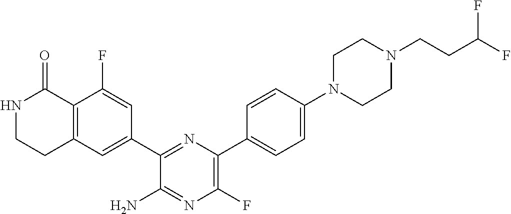 [SMILES: Nc1nc(F)c(-c2ccc(N3CCN(CCC(F)F)CC3)cc2)nc1-c1cc(F)c2c(c1)CCNC2=O] |
| I-83 | 6-(3-amino-6-(3- ((dimethylamino)methyl)-4- (tetrahydro-2H-pyran-4- yl)phenyl)-5-fluoropyrazin-2-yl)- 8-fluoro-3,4-dihydroisoquinolin- 1(2H)-one | 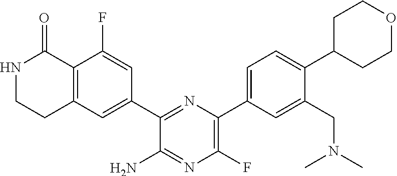 [SMILES: CN(C)Cc1cc(-c2nc(-c3cc(F)c4c(c3)CCNC4=O)c(N)nc2F)ccc1C1CCOCC1] |
| I-84 | 6-(3-amino-6-(4-(4- (cyclopropylmethyl)piperazin-1- yl)phenyl)-5-fluoropyrazin-2-yl)- 4-fluoroisoquinolin-1(2H)-one | 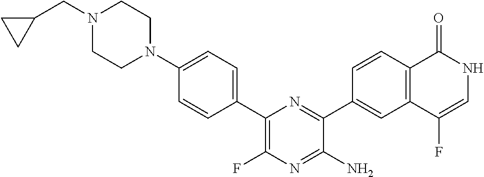 [SMILES: Nc1nc(F)c(-c2ccc(N3CCN(CC4CC4)CC3)cc2)nc1-c1ccc2c(=O)[nH]cc(F)c2c1] |
| I-85 | 6-(3-amino-5-fluoro-6-(4-(4- isopropylpiperazin-1- yl)phenyl)pyrazin-2-yl)-4,8- difluoroisoquinolin-1(2H)-one | 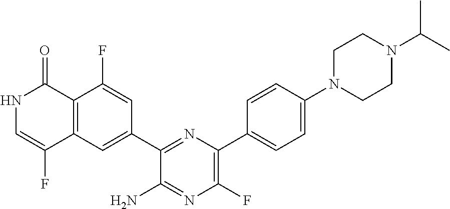 [SMILES: CC(C)N1CCN(c2ccc(-c3nc(-c4cc(F)c5c(=O)[nH]cc(F)c5c4)c(N)nc3F)cc2)CC1] |
| I-86 | 6-(3-amino-6-(4-(4-(3,3- difluoropropyl)piperazin-1- yl)phenyl)-5-fluoropyrazin-2- yl)isoquinolin-1(2H)-one | 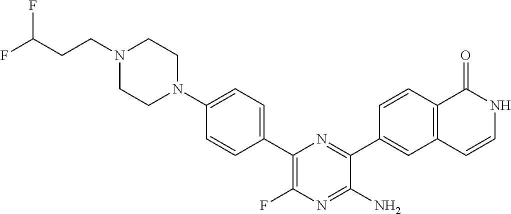 [SMILES: Nc1nc(F)c(-c2ccc(N3CCN(CCC(F)F)CC3)cc2)nc1-c1ccc2c(=O)[nH]ccc2c1] |
| I-87 | 6-(3-amino-6-(4-(4-(3,3- difluoropropyl)piperazin-1- yl)phenyl)-5-fluoropyrazin-2-yl)- 4-methylisoquinolin-1(2H)-one | 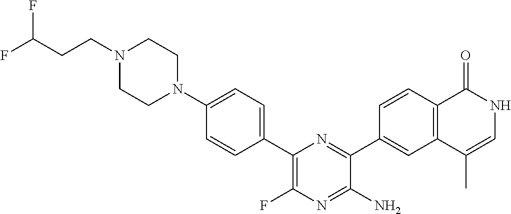 [SMILES: Cc1c[nH]c(=O)c2ccc(-c3nc(-c4ccc(N5CCN(CCC(F)F)CC5)cc4)c(F)nc3N)cc12] |
| I-88 | 6-(3-amino-6-(3- ((dimethylamino)methyl)-4- morpholinophenyl)-5- fluoropyrazin-2-yl)-8-fluoro-3,4- dihydroisoquinolin-1(2H)-one | 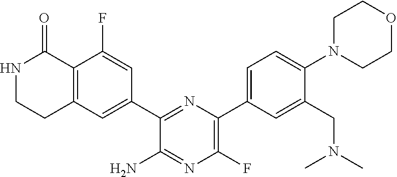 [SMILES: CN(C)Cc1cc(-c2nc(-c3cc(F)c4c(c3)CCNC4=O)c(N)nc2F)ccc1N1CCOCC1] |
| I-89 | 6-(3-amino-6-(3- ((dimethylamino)methyl)-4- morpholinophenyl)-5- fluoropyrazin-2-yl)-4- fluoroisoquinolin-1(2H)-one | 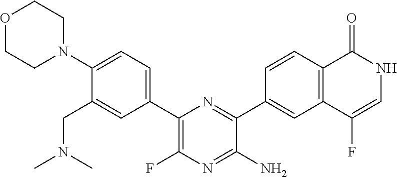 [SMILES: CN(C)Cc1cc(-c2nc(-c3ccc4c(=O)[nH]cc(F)c4c3)c(N)nc2F)ccc1N1CCOCC1] |
| I-90 | 6-(3-amino-5-fluoro-6-(4-(4- isopropylpiperazin-1- yl)phenyl)pyrazin-2-yl)-4- chloro-8-fluoroisoquinolin- 1(2H)-one | 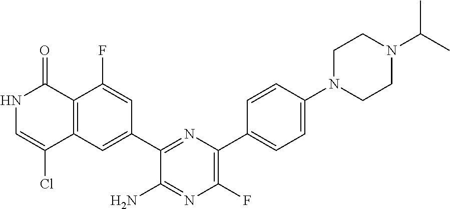 [SMILES: CC(C)N1CCN(c2ccc(-c3nc(-c4cc(F)c5c(=O)[nH]cc(Cl)c5c4)c(N)nc3F)cc2)CC1] |
| I-91 | 7-(3-amino-6-(3- ((dimethylamino)methyl)-4- (tetrahydro-2H-pyran-4- yl)phenyl)-5-fluoropyrazin-2-yl)- 5-fluoro-2-methylquinazolin- 4(3H)-one | 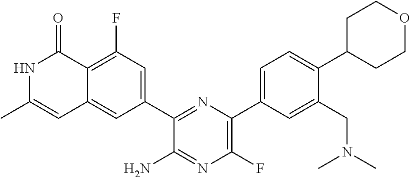 [SMILES: Cc1cc2cc(-c3nc(-c4ccc(C5CCOCC5)c(CN(C)C)c4)c(F)nc3N)cc(F)c2c(=O)[nH]1] |
| I-92 | 7-(3-amino-6-(3- ((dimethylamino)methyl)-4- (tetrahydro-2H-pyran-4- yl)phenyl)-5-fluoropyrazin-2-yl)- 2-methylquinazolin-4(3H)-one | 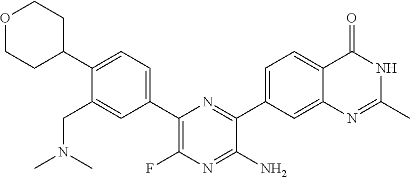 [SMILES: Cc1nc2cc(-c3nc(-c4ccc(C5CCOCC5)c(CN(C)C)c4)c(F)nc3N)ccc2c(=O)[nH]1] |
| I-93 | 6-(3-amino-5-fluoro-6-(4- ((3R,5S)-3,4,5- trimethylpiperazin-1- yl)phenyl)pyrazin-2-yl)-4- fluoroisoquinolin-1(2H)-one | 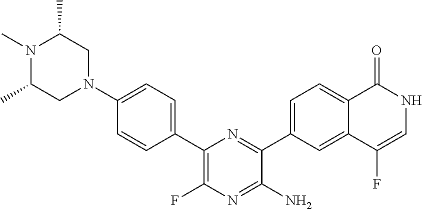 [SMILES: C[C@@H]1CN(c2ccc(-c3nc(-c4ccc5c(=O)[nH]cc(F)c5c4)c(N)nc3F)cc2)C[C@H](C)N1C] |
| I-94 | 6-(3-amino-5-fluoro-6-(4- ((3R,5S)-3,4,5- trimethylpiperazin-1- yl)phenyl)pyrazin-2-yl)-4- methylisoquinolin-1(2H)-one | 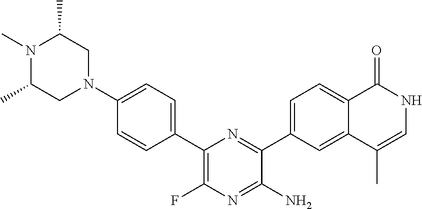 [SMILES: Cc1c[nH]c(=O)c2ccc(-c3nc(-c4ccc(N5C[C@@H](C)N(C)[C@@H](C)C5)cc4)c(F)nc3N)cc12] |
| I-95 | 7-(3-amino-5-fluoro-6-(4- ((3R,5S)-3,4,5- trimethylpiperazin-1- yl)phenyl)pyrazin-2-yl)-2- methylquinazolin-4(3H)-one | 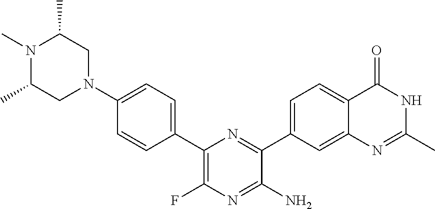 |
| I-96 | 6-(3-amino-6-(3- ((dimethylamino)methyl)-4- morpholinophenyl)-5- fluoropyrazin-2-yl)-4- methylisoquinolin-1(2H)-one | 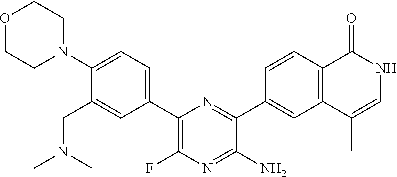 [SMILES: Cc1c[nH]c(=O)c2ccc(-c3nc(-c4ccc(N5CCOCC5)c(CN(C)C)c4)c(F)nc3N)cc12] |
| I-97 | 6-(3-amino-6-(3- ((dimethylamino)methyl)-4- (tetrahydro-2H-pyran-4- yl)phenyl)-5-fluoropyrazin-2-yl)- 4-fluoro-3-methylisoquinolin- 1(2H)-one | 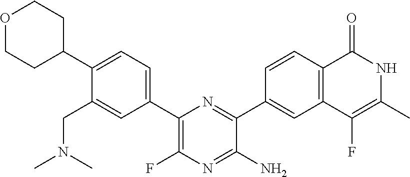 [SMILES: Cc1[nH]c(=O)c2ccc(-c3nc(-c4ccc(C5CCOCC5)c(CN(C)C)c4)c(F)nc3N)cc2c1F] |
| I-98 | 6-(3-amino-6-(3- ((dimethylamino)methyl)-4- (tetrahydro-2H-pyran-4- yl)phenyl)-5-fluoropyrazin-2-yl)- 4-methylisoquinolin-1(2H)-one | 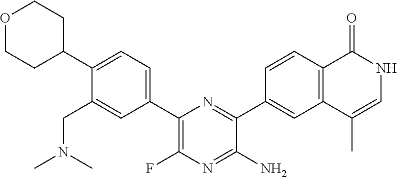 [SMILES: Cc1c[nH]c(=O)c2ccc(-c3nc(-c4ccc(C5CCOCC5)c(CN(C)C)c4)c(F)nc3N)cc12] |
| I-99 | 6-(3-amino-6-(4-((1S,5R)-3- cyclobutyl-3- azabicyclo[3.1.0]hexan-1- yl)phenyl)-5-fluoropyrazin-2-yl)- 4-fluoroisoquinolin-1(2H)-one | 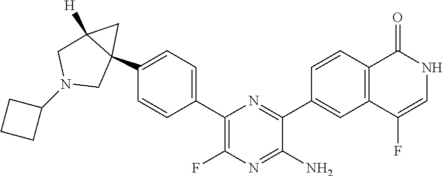 [SMILES: Nc1nc(F)c(-c2ccc([C@]34C[C@H]3CN(C3CCC3)C4)cc2)nc1-c1ccc2c(=O)[nH]cc(F)c2c1] |
| I-100 | 6-(3-amino-6-(3- ((dimethylamino)methyl)-4- (tetrahydro-2H-pyran-4- yl)phenyl)-5-fluoropyrazin-2-yl)- 8-fluoro-3-methylisoquinolin- 1(2H)-one | 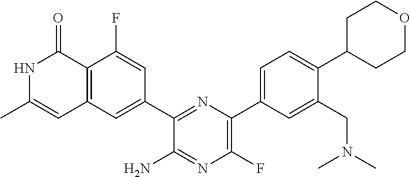 [SMILES: Cc1cc2cc(-c3nc(-c4ccc(C5CCOCC5)c(CN(C)C)c4)c(F)nc3N)cc(F)c2c(=O)[nH]1] |
| I-101 | 6-(3-amino-6-(3- ((dimethylamino)methyl)-4- morpholinophenyl)-5- fluoropyrazin-2-yl)-4-fluoro-3- methylisoquinolin-1(2H)-one | 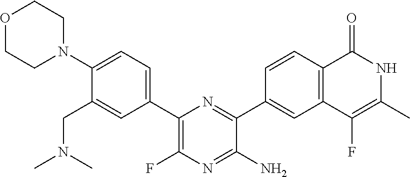 [SMILES: Cc1[nH]c(=O)c2ccc(-c3nc(-c4ccc(N5CCOCC5)c(CN(C)C)c4)c(F)nc3N)cc2c1F] |
| I-102 | 6-(3-amino-5-fluoro-6-(4-(4- isopropylpiperazin-1- yl)phenyl)pyrazin-2-yl)-4- chloro-8-fluoro-3- methylisoquinolin-1(2H)-one | 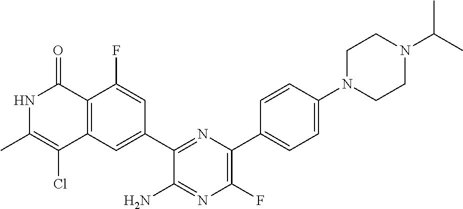 [SMILES: Cc1[nH]c(=O)c2c(F)cc(-c3nc(-c4ccc(N5CCN(C(C)C)CC5)cc4)c(F)nc3N)cc2c1Cl] |
| I-103 | 6′-(3-amino-5-fluoro-6-(4-(4- isopropylpiperazin-1- yl)phenyl)pyrazin-2-yl)-2′,3′- dihydro-1′H- spiro[cyclopropane-1,4′- isoquinolin]-1′-one | 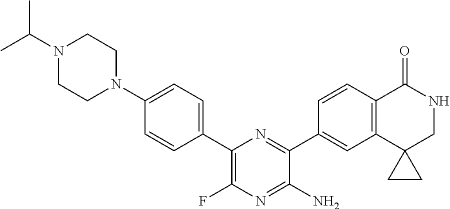 [SMILES: CC(C)N1CCN(c2ccc(-c3nc(-c4ccc5c(c4)C4(CC4)CNC5=O)c(N)nc3F)cc2)CC1] |
| I-104 | 6-(3-amino-6-(3- ((dimethylamino)methyl)-4- (tetrahydro-2H-pyran-4- yl)phenyl)-5-fluoropyrazin-2-yl)- 4-chloro-8-fluoro-3- methylisoquinolin-1(2H)-one | 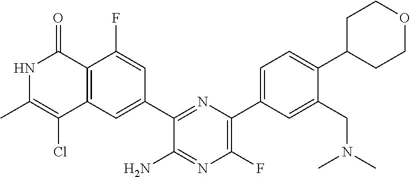 [SMILES: Cc1[nH]c(=O)c2c(F)cc(-c3nc(-c4ccc(C5CCOCC5)c(CN(C)C)c4)c(F)nc3N)cc2c1Cl] |
| I-105 | 6′-(3-amino-6-(3- ((dimethylamino)methyl)-4- morpholinophenyl)-5- fluoropyrazin-2-yl)-2′,3′- dihydro-1′H- spiro[cyclopropane-1,4′- isoquinolin]-1′-one | 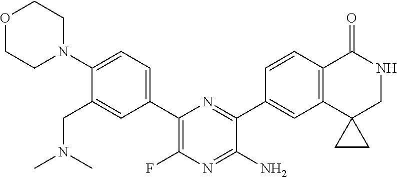 [SMILES: CN(C)Cc1cc(-c2nc(-c3ccc4c(c3)C3(CC3)CNC4=O)c(N)nc2F)ccc1N1CCOCC1] |
| I-106 | 6-(3-amino-6-(4-(4- (cyclopropylmethyl)piperazin-1- yl)phenyl)-5-fluoropyrazin-2-yl)- 4-fluoro-3-methylisoquinolin- 1(2H)-one | 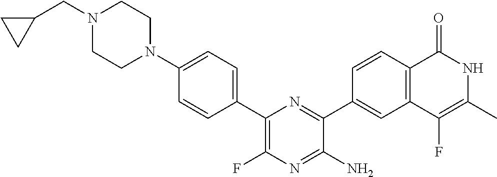 [SMILES: Cc1[nH]c(=O)c2ccc(-c3nc(-c4ccc(N5CCN(CC6CC6)CC5)cc4)c(F)nc3N)cc2c1F] |
| I-107 | 6-(3-amino-6-(4-(4- (cyclopropylmethyl)piperazin-1- yl)phenyl)-5-fluoropyrazin-2-yl)- 4-chloro-8-fluoro-3- methylisoquinolin-1(2H)-one | 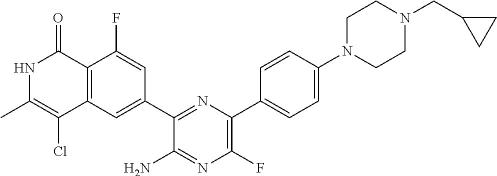 [SMILES: Cc1[nH]c(=O)c2c(F)cc(-c3nc(-c4ccc(N5CCN(CC6CC6)CC5)cc4)c(F)nc3N)cc2c1Cl] |
| I-108 | 6′-(3-amino-6-(4-(4- (cyclopropylmethyl)piperazin-1- yl)phenyl)-5-fluoropyrazin-2-yl)- 2′,3′-dihydro-1′H- spiro[cyclopropane-1,4′- isoquinolin]-1′-one | 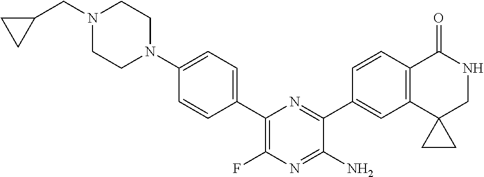 [SMILES: Nc1nc(F)c(-c2ccc(N3CCN(CC4CC4)CC3)cc2)nc1-c1ccc2c(c1)C1(CC1)CNC2=O] |
| I-109 | 6-(3-amino-6-(3- ((dimethylamino)methyl)-4- morpholinophenyl)-5- fluoropyrazin-2-yl)-4-chloro-8- fluoro-3-methylisoquinolin- 1(2H)-one | 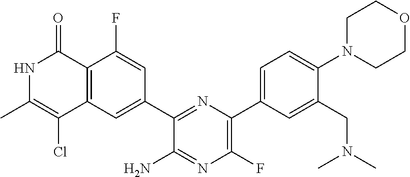 [SMILES: Cc1[nH]c(=O)c2c(F)cc(-c3nc(-c4ccc(N5CCOCC5)c(CN(C)C)c4)c(F)nc3N)cc2c1Cl] |
| I-110 | 6′-(3-amino-6-(3- ((dimethylamino)methyl)-4- (tetrahydro-2H-pyran-4- yl)phenyl)-5-fluoropyrazin-2-yl)- 2′, 3′-dihydro-1′H- spiro[cyclopropane-1,4′- isoquinolin]-1′-one | 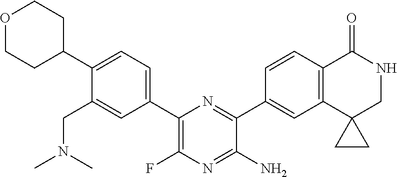 [SMILES: CN(C)Cc1cc(-c2nc(-c3ccc4c(c3)C3(CC3)CNC4=O)c(N)nc2F)ccc1C1CCOCC1] |
| I-111 | 6-(3-amino-5-fluoro-6-(4-(4- isopropylpiperazin-1- yl)phenyl)pyrazin-2-yl)-4,8- difluoro-3-methylisoquinolin- 1(2H)-one | 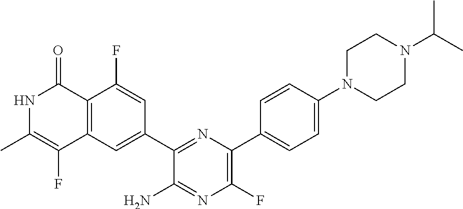 [SMILES: Cc1[nH]c(=O)c2c(F)cc(-c3nc(-c4ccc(N5CCN(C(C)C)CC5)cc4)c(F)nc3N)cc2c1F] |
| I-112 | 6-(3-amino-5-fluoro-6-(4- morpholino-3-(pyrrolidin-1- ylmethyl)phenyl)pyrazin-2-yl)- 8-fluoro-3,4-dihydroisoquinolin- 1(2H)-one | 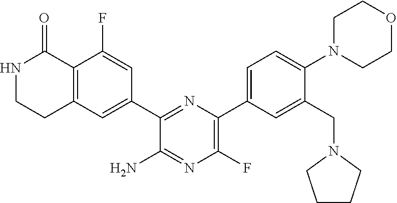 [SMILES: Nc1nc(F)c(-c2ccc(N3CCOCC3)c(CN3CCCC3)c2)nc1-c1cc(F)c2c(c1)CCNC2=O] |
| I-113 | 6-(3-amino-5-fluoro-6-(4- morpholino-3-(pyrrolidin-1- ylmethyl)phenyl)pyrazin-2-yl)- 3,4-dihydroisoquinolin-1(2H)- one | 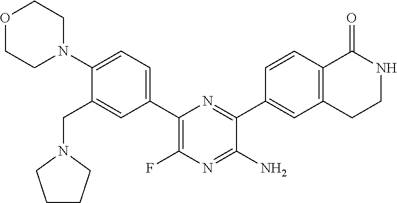 [SMILES: Nc1nc(F)c(-c2ccc(N3CCOCC3)c(CN3CCCC3)c2)nc1-c1ccc2c(c1)CCNC2=O] |
| I-114 | 6-(3-amino-5-fluoro-6-(4- morpholino-3-(pyrrolidin-1- ylmethyl)phenyl)pyrazin-2- yl)isoquinolin-1(2H)-one | 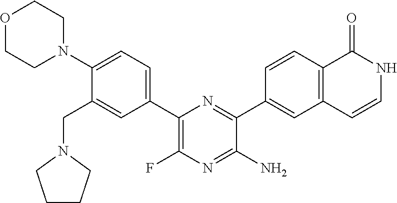 [SMILES: Nc1nc(F)c(-c2ccc(N3CCOCC3)c(CN3CCCC3)c2)nc1-c1ccc2c(=O)[nH]ccc2c1] |
| I-115 | 6-(3-amino-6-(4-(4- (cyclopropylmethyl)piperazin-1- yl)phenyl)-5-fluoropyrazin-2-yl)- 4,8-difluoro-3- methylisoquinolin-1(2H)-one | 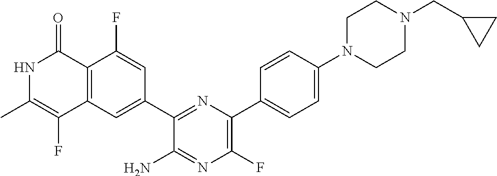 [SMILES: Cc1[nH]c(=O)c2c(F)cc(-c3nc(-c4ccc(N5CCN(CC6CC6)CC5)cc4)c(F)nc3N)cc2c1F] |
| I-116 | 6-(3-amino-6-(3- ((dimethylamino)methyl)-4- morpholinophenyl)-5- fluoropyrazin-2-yl)-4,8-difluoro- 3-methylisoquinolin-1(2H)-one | 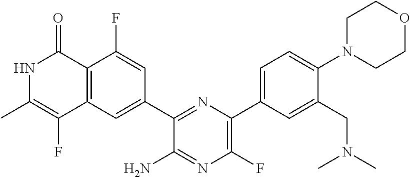 [SMILES: Cc1[nH]c(=O)c2c(F)cc(-c3nc(-c4ccc(N5CCOCC5)c(CN(C)C)c4)c(F)nc3N)cc2c1F] |
| I-117 | 6-(3-amino-6-(3- ((dimethylamino)methyl)-4- (tetrahydro-2H-pyran-4- yl)phenyl)-5-fluoropyrazin-2-yl)- 4,8-difluoro-3- methylisoquinolin-1(2H)-one | 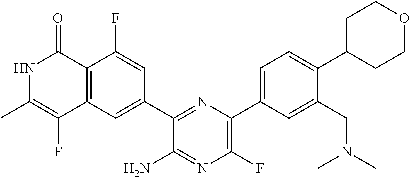 [SMILES: Cc1[nH]c(=O)c2c(F)cc(-c3nc(-c4ccc(C5CCOCC5)c(CN(C)C)c4)c(F)nc3N)cc2c1F] |
| I-118 | 6-(3-amino-5-fluoro-6-(3-((4- methoxypiperidin-1-yl)methyl)- 4-morpholinophenyl)pyrazin-2- yl)-7-fluoro-3,4- dihydroisoquinolin-1(2H)-one | 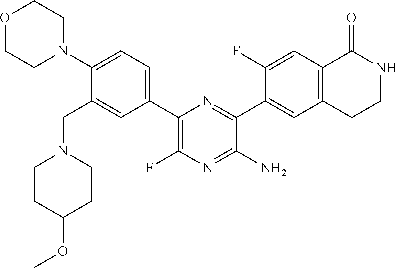 [SMILES: COC1CCN(Cc2cc(-c3nc(-c4cc5c(cc4F)C(=O)NCC5)c(N)nc3F)ccc2N2CCOCC2)CC1] |
| I-119 | 6-(3-amino-5-fluoro-6-(4-(4- isopropylpiperazin-1- yl)phenyl)pyrazin-2-yl)-4- methoxyisoquinolin-1(2H)-one | 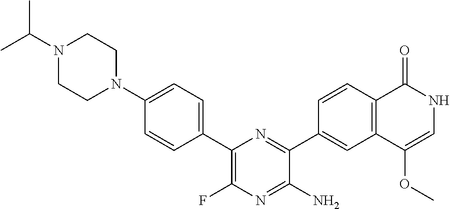 [SMILES: COc1c[nH]c(=O)c2ccc(-c3nc(-c4ccc(N5CCN(C(C)C)CC5)cc4)c(F)nc3N)cc12] |
| I-120 | 6-(3-amino-6-(3- ((dimethylamino)methyl)-4- morpholinophenyl)-5- fluoropyrazin-2-yl)-4,8- difluoroisoquinolin-1(2H)-one | 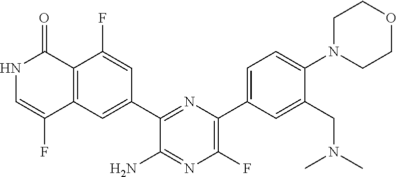 [SMILES: CN(C)Cc1cc(-c2nc(-c3cc(F)c4c(=O)[nH]cc(F)c4c3)c(N)nc2F)ccc1N1CCOCC1] |
| I-121 | 6-(3-amino-6-(3- ((dimethylamino)methyl)-4- (tetrahydro-2H-pyran-4- yl)phenyl)-5-fluoropyrazin-2-yl)- 4,8-difluoroisoquinolin-1(2H)- one | 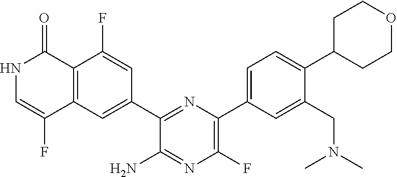 [SMILES: CN(C)Cc1cc(-c2nc(-c3cc(F)c4c(=O)[nH]cc(F)c4c3)c(N)nc2F)ccc1C1CCOCC1] |
| I-122 | 6-(3-amino-6-(4-(4- (cyclopropylmethyl)piperazin-1- yl)phenyl)-5-fluoropyrazin-2-yl)- 4-chloro-3-methylisoquinolin- 1(2H)-one | 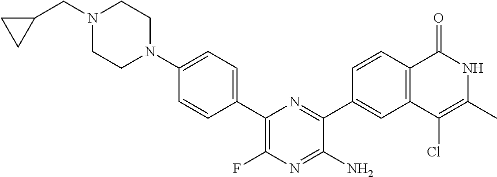 [SMILES: Cc1[nH]c(=O)c2ccc(-c3nc(-c4ccc(N5CCN(CC6CC6)CC5)cc4)c(F)nc3N)cc2c1Cl] |
| I-123 | 6-(3-amino-5-fluoro-6-(4-(8- methyl-5-oxa-2,8- diazaspiro[3.5]nonan-2- yl)phenyl)pyrazin-2-yl)-4-fluoro- 3-methylisoquinolin-1(2H)-one | 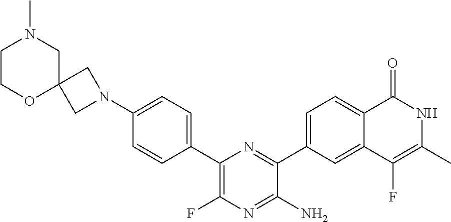 [SMILES: Cc1[nH]c(=O)c2ccc(-c3nc(-c4ccc(N5CC6(CN(C)CCO6)C5)cc4)c(F)nc3N)cc2c1F] |
| I-124 | 6-(3-amino-6-(3- ((dimethylamino)methyl)-4- morpholinophenyl)-5- fluoropyrazin-2-yl)-4-chloro-3- methylisoquinolin-1(2H)-one | 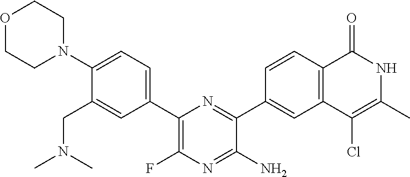 [SMILES: Cc1[nH]c(=O)c2ccc(-c3nc(-c4ccc(N5CCOCC5)c(CN(C)C)c4)c(F)nc3N)cc2c1Cl] |
| I-125 | 6-(3-amino-6-(3- ((dimethylamino)methyl)-4- (tetrahydro-2H-pyran-4- yl)phenyl)-5-fluoropyrazin-2-yl)- 4-chloro-3-methylisoquinolin- 1(2H)-one | 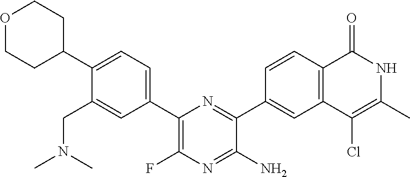 [SMILES: Cc1[nH]c(=O)c2ccc(-c3nc(-c4ccc(C5CCOCC5)c(CN(C)C)c4)c(F)nc3N)cc2c1Cl] |
| I-126 | 6-(3-amino-5-fluoro-6-(3-((4- methoxypiperidin-1-yl)methyl)- 4- thiomorpholinophenyl)pyrazin- 2-yl)-3,4-dihydroisoquinolin- 1(2H)-one | 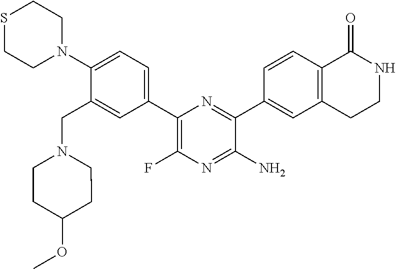 [SMILES: COC1CCN(Cc2cc(-c3nc(-c4ccc5c(c4)CCNC5=O)c(N)nc3F)ccc2N2CCSCC2)CC1] |
| I-127 | 6-(3-amino-5-fluoro-6-(3-((4- methoxypiperidin-1-yl)methyl)- 4- thiomorpholinophenyl)pyrazin- 2-yl)-7-fluoro-3,4- dihydroisoquinolin-1(2H)-one | 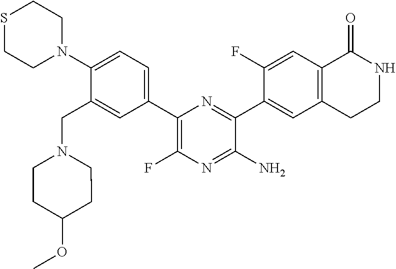 [SMILES: COC1CCN(Cc2cc(-c3nc(-c4cc5c(cc4F)C(=O)NCC5)c(N)nc3F)ccc2N2CCSCC2)CC1] |
| I-128 | 6-(3-amino-6-(3- ((dimethylamino)methyl)-4- thiomorpholinophenyl)-5- fluoropyrazin-2-yl)-3,4- dihydroisoquinolin-1(2H)-one | 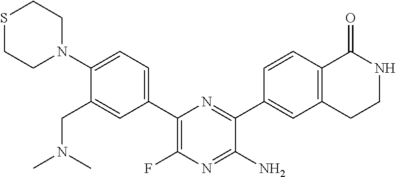 [SMILES: CN(C)Cc1cc(-c2nc(-c3ccc4c(c3)CCNC4=O)c(N)nc2F)ccc1N1CCSCC1] |
| I-129 | 6-(3-amino-6-(3- ((dimethylamino)methyl)-4- thiomorpholinophenyl)-5- fluoropyrazin-2-yl)-7-fluoro-3,4- dihydroisoquinolin-1(2H)-one | 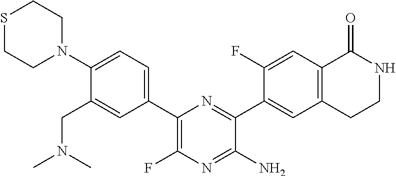 [SMILES: CN(C)Cc1cc(-c2nc(-c3cc4c(cc3F)C(=O)NCC4)c(N)nc2F)ccc1N1CCSCC1] |
| I-130 | 7-(3-amino-5-fluoro-6-(4-(8- methyl-5-oxa-2,8- diazaspiro[3.5]nonan-2- yl)phenyl)pyrazin-2-yl)-2- methylquinazolin-4(3H)-one | 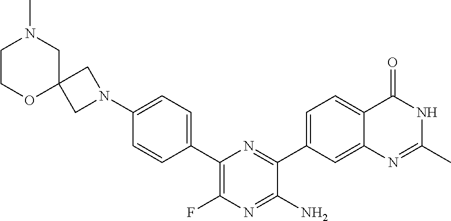 [SMILES: Cc1nc2cc(-c3nc(-c4ccc(N5CC6(CN(C)CCO6)C5)cc4)c(F)nc3N)ccc2c(=O)[nH]1] |
| I-131 | 6-(3-amino-5-fluoro-6-(3-((4- methoxypiperidin-1-yl)methyl)- 4-(tetrahydro-2H-pyran-4- yl)phenyl)pyrazin-2-yl)-3,4- dihydroisoquinolin-1(2H)-one | 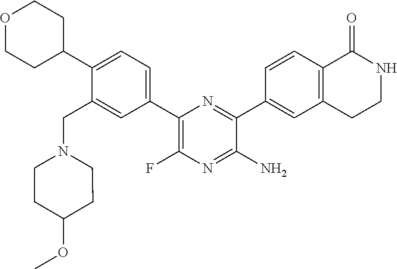 [SMILES: COC1CCN(Cc2cc(-c3nc(-c4ccc5c(c4)CCNC5=O)c(N)nc3F)ccc2C2CCOCC2)CC1] |
| I-132 | 6-(3-amino-5-fluoro-6-(3-((4- methoxypiperidin-1-yl)methyl)- 4-(tetrahydro-2H-pyran-4- yl)phenyl)pyrazin-2-yl)-7-fluoro- 3,4-dihydroisoquinolin-1(2H)- one | 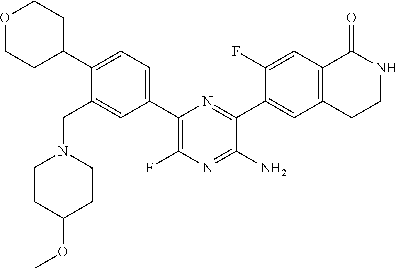 [SMILES: COC1CCN(Cc2cc(-c3nc(-c4cc5c(cc4F)C(=O)NCC5)c(N)nc3F)ccc2C2CCOCC2)CC1] |
| I-133 | 6-(3-amino-5-fluoro-6-(3-((3- methoxypyrrolidin-1-yl)methyl)- 4-morpholinophenyl)pyrazin-2- yl)-3,4-dihydroisoquinolin- 1(2H)-one | 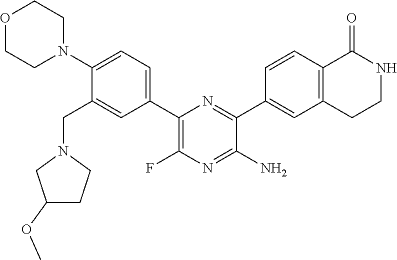 [SMILES: COC1CCN(Cc2cc(-c3nc(-c4ccc5c(c4)CCNC5=O)c(N)nc3F)ccc2N2CCOCC2)C1] |
| I-134 | 6-(3-amino-5-fluoro-6-(3-((3- methoxypyrrolidin-1-yl)methyl)- 4-morpholinophenyl)pyrazin-2- yl)-7-fluoro-3,4- dihydroisoquinolin-1(2H)-one | 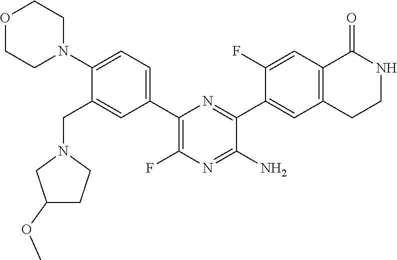 [SMILES: COC1CCN(Cc2cc(-c3nc(-c4cc5c(cc4F)C(=O)NCC5)c(N)nc3F)ccc2N2CCOCC2)C1] |
| I-135 | 6-(3-amino-5-fluoro-6-(4- ((1S,5R)-3-methyl-3- azabicyclo[3.1.0]hexan-1- yl)phenyl)pyrazin-2-yl)-4,4,8- trifluoro-3-methyl-3,4- dihydroisoquinolin-1(2H)-one | 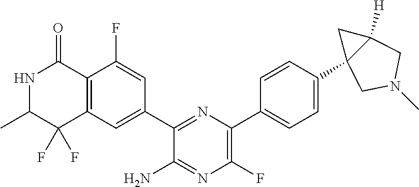 |
| I-136 | 6-(3-amino-6-(4-(4-(4,4- difluorobutyl)piperazin-1- yl)phenyl)-5-fluoropyrazin-2-yl)- 4-fluoroisoquinolin-1(2H)-one | 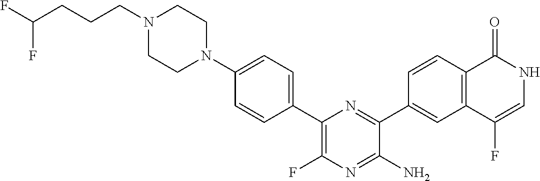 [SMILES: Nc1nc(F)c(-c2ccc(N3CCN(CCCC(F)F)CC3)cc2)nc1-c1ccc2c(=O)[nH]cc(F)c2c1] |
| I-137 | 6-(3-amino-6-(4-(4-(4,4- difluorobutyl)piperazin-1- yl)phenyl)-5-fluoropyrazin-2-yl)- 8-fluoro-3,4-dihydroisoquinolin- 1(2H)-one | 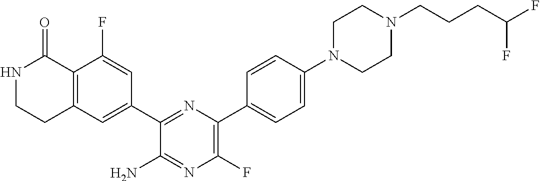 [SMILES: Nc1nc(F)c(-c2ccc(N3CCN(CCCC(F)F)CC3)cc2)nc1-c1cc(F)c2c(c1)CCNC2=O] |
| I-138 | 6-(3-amino-5-fluoro-6-(3-((4- methoxypiperidin-1-yl)methyl)- 4-morpholinophenyl)pyrazin-2- yl)-3,4-dihydroisoquinolin- 1(2H)-one | 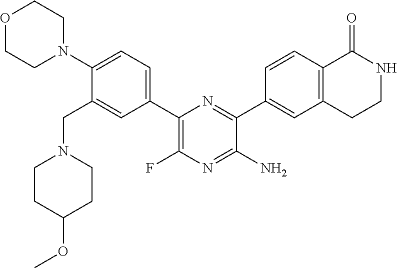 [SMILES: COC1CCN(Cc2cc(-c3nc(-c4ccc5c(c4)CCNC5=O)c(N)nc3F)ccc2N2CCOCC2)CC1] |
| I-139 | 6-(3-amino-6-(4-(4- cyclobutylpiperazin-1- yl)phenyl)-5-fluoropyrazin-2-yl)- 4-methylisoquinolin-1(2H)-one | 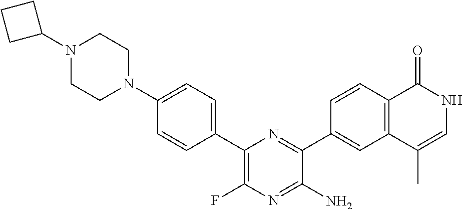 [SMILES: Cc1c[nH]c(=O)c2ccc(-c3nc(-c4ccc(N5CCN(C6CCC6)CC5)cc4)c(F)nc3N)cc12] |
| I-140 | 6-(3-amino-5-fluoro-6-(4-(4- isopropylpiperazin-1- yl)phenyl)pyrazin-2-yl)-4,7- difluoroisoquinolin-1(2H)-one | 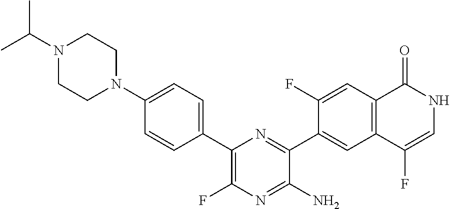 [SMILES: CC(C)N1CCN(c2ccc(-c3nc(-c4cc5c(F)c[nH]c(=O)c5cc4F)c(N)nc3F)cc2)CC1] |
| I-141 | 6-(3-amino-6-(4-(4- (cyclopropylmethyl)piperazin-1- yl)phenyl)-5-fluoropyrazin-2-yl)- 4,7-difluoroisoquinolin-1(2H)- one | 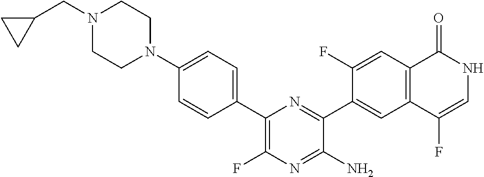 [SMILES: Nc1nc(F)c(-c2ccc(N3CCN(CC4CC4)CC3)cc2)nc1-c1cc2c(F)c[nH]c(=O)c2cc1F] |
| I-142 | 6-(3-amino-6-(3- ((dimethylamino)methyl)-4- morpholinophenyl)-5- fluoropyrazin-2-yl)-4,7- difluoroisoquinolin-1(2H)-one | 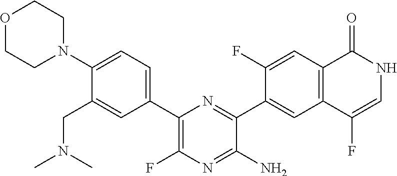 [SMILES: CN(C)Cc1cc(-c2nc(-c3cc4c(F)c[nH]c(=O)c4cc3F)c(N)nc2F)ccc1N1CCOCC1] |
| I-143 | 6-(3-amino-6-(3- ((dimethylamino)methyl)-4- (tetrahydro-2H-pyran-4- yl)phenyl)-5-fluoropyrazin-2-yl)- 4,7-difluoroisoquinolin-1(2H)- one | 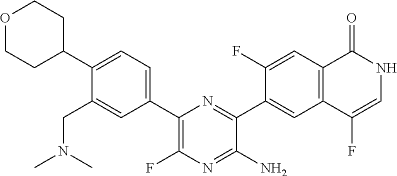 [SMILES: CN(C)Cc1cc(-c2nc(-c3cc4c(F)c[nH]c(=O)c4cc3F)c(N)nc2F)ccc1C1CCOCC1] |
| I-144 | 6-(3-amino-6-(3-(azetidin-1- ylmethyl)-4-morpholinophenyl)- 5-fluoropyrazin-2-yl)-3,4- dihydroisoquinolin-1(2H)-one | 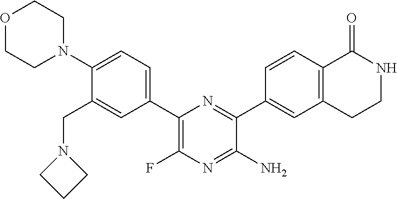 [SMILES: Nc1nc(F)c(-c2ccc(N3CCOCC3)c(CN3CCC3)c2)nc1-c1ccc2c(c1)CCNC2=O] |
| I-145 | 6-(3-amino-6-(3-(azetidin-1- ylmethyl)-4-morpholinophenyl)- 5-fluoropyrazin-2-yl)-7-fluoro- 3,4-dihydroisoquinolin-1(2H)- one | 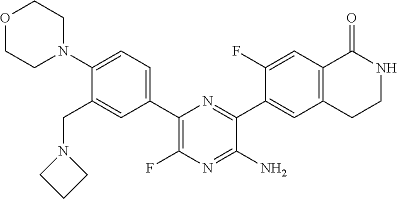 [SMILES: Nc1nc(F)c(-c2ccc(N3CCOCC3)c(CN3CCC3)c2)nc1-c1cc2c(cc1F)C(=O)NCC2] |
| I-146 | 6-(3-amino-6-(3-(azetidin-1- ylmethyl)-4-morpholinophenyl)- 5-fluoropyrazin-2-yl)-8-fluoro- 3,4-dihydroisoquinolin-1(2H)- one | 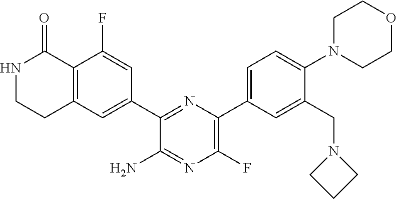 [SMILES: Nc1nc(F)c(-c2ccc(N3CCOCC3)c(CN3CCC3)c2)nc1-c1cc(F)c2c(c1)CCNC2=O] |
| I-147 | 6-(3-amino-5-fluoro-6-(3- (pyrrolidin-1-ylmethyl)-4- (tetrahydro-2H-pyran-4- yl)phenyl)pyrazin-2-yl)-3,4- dihydroisoquinolin-1(2H)-one | 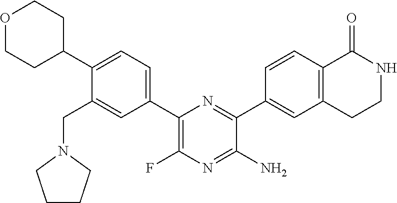 [SMILES: Nc1nc(F)c(-c2ccc(C3CCOCC3)c(CN3CCCC3)c2)nc1-c1ccc2c(c1)CCNC2=O] |
| I-148 | 6-(3-amino-5-fluoro-6-(3- (pyrrolidin-1-ylmethyl)-4- (tetrahydro-2H-pyran-4- yl)phenyl)pyrazin-2-yl)-7-fluoro- 3,4-dihydroisoquinolin-1(2H)- one | 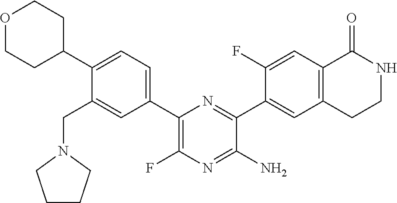 [SMILES: Nc1nc(F)c(-c2ccc(C3CCOCC3)c(CN3CCCC3)c2)nc1-c1cc2c(cc1F)C(=O)NCC2] |
| I-149 | 6-(3-amino-5-fluoro-6-(3- (pyrrolidin-1-ylmethyl)-4- (tetrahydro-2H-pyran-4- yl)phenyl)pyrazin-2-yl)-8-fluoro- 3,4-dihydroisoquinolin-1(2H)- one | 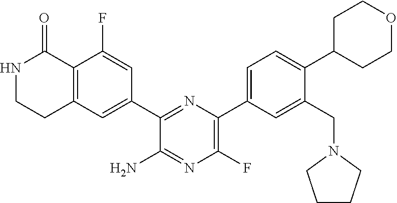 [SMILES: Nc1nc(F)c(-c2ccc(C3CCOCC3)c(CN3CCCC3)c2)nc1-c1cc(F)c2c(c1)CCNC2=O] |
| I-150 | 6-(3-amino-6-(4-(4- cyclobutylpiperazin-1- yl)phenyl)-5-fluoropyrazin-2-yl)- 4,4,8-trifluoro-3-methyl-3,4- dihydroisoquinolin-1(2H)-one | 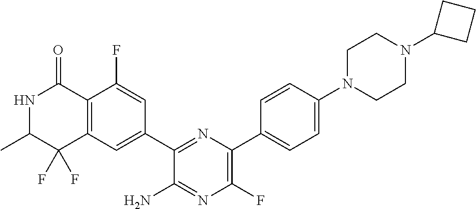 [SMILES: CC1NC(=O)c2c(F)cc(-c3nc(-c4ccc(N5CCN(C6CCC6)CC5)cc4)c(F)nc3N)cc2C1(F)F] |
| I-151 | (R)-6-(3-amino-5-fluoro-6-(3- ((3-methoxypyrrolidin-1- yl)methyl)-4- morpholinophenyl)pyrazin-2- yl)-3,4-dihydroisoquinolin- 1(2H)-one | 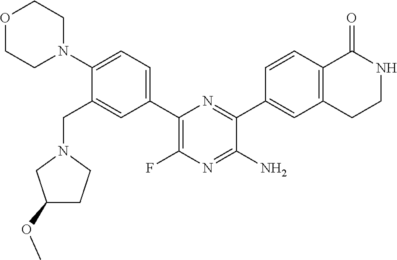 [SMILES: CO[C@@H]1CCN(Cc2cc(-c3nc(-c4ccc5c(c4)CCNC5=O)c(N)nc3F)ccc2N2CCOCC2)C1] |
| I-152 | (R)-6-(3-amino-5-fluoro-6-(3- ((3-methoxypyrrolidin-1- yl)methyl)-4- morpholinophenyl)pyrazin-2- yl)-7-fluoro-3,4- dihydroisoquinolin-1(2H)-one | 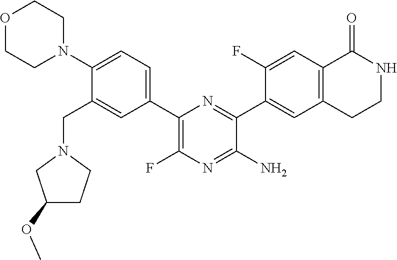 [SMILES: CO[C@@H]1CCN(Cc2cc(-c3nc(-c4cc5c(cc4F)C(=O)NCC5)c(N)nc3F)ccc2N2CCOCC2)C1] |
| I-153 | 6-(3-amino-6-(3- ((dimethylamino)methyl)-4- morpholinophenyl)-5- fluoropyrazin-2-yl)-4- chloroisoquinolin-1(2H)-one | 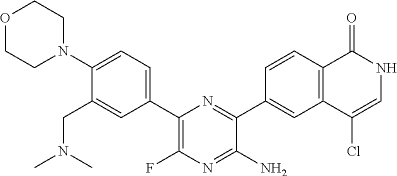 [SMILES: CN(C)Cc1cc(-c2nc(-c3ccc4c(=O)[nH]cc(Cl)c4c3)c(N)nc2F)ccc1N1CCOCC1] |
| I-154 | 6-(3-amino-6-(3- ((dimethylamino)methyl)-4- (tetrahydro-2H-pyran-4- yl)phenyl)-5-fluoropyrazin-2-yl)- 4-chloroisoquinolin-1(2H)-one | 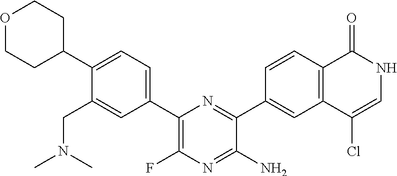 [SMILES: CN(C)Cc1cc(-c2nc(-c3ccc4c(=O)[nH]cc(Cl)c4c3)c(N)nc2F)ccc1C1CCOCC1] |
| I-155 | (R)-6-(3-amino-5-fluoro-6-(3- ((3-methoxypyrrolidin-1- yl)methyl)-4- morpholinophenyl)pyrazin-2- yl)-8-fluoro-3,4- dihydroisoquinolin-1(2H)-one | 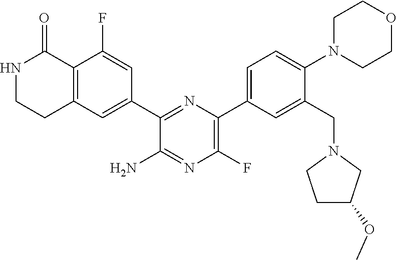 [SMILES: CO[C@@H]1CCN(Cc2cc(-c3nc(-c4cc(F)c5c(c4)CCNC5=O)c(N)nc3F)ccc2N2CCOCC2)C1] |
| I-156 | (S)-6-(3-amino-5-fluoro-6-(3- ((3-methoxypyrrolidin-1- yl)methyl)-4- morpholinophenyl)pyrazin-2- yl)-3,4-dihydroisoquinolin- 1(2H)-one | 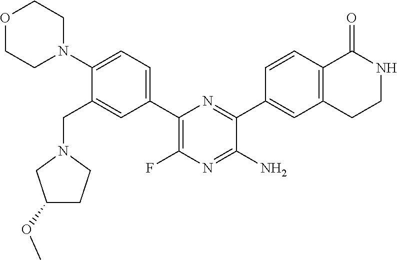 [SMILES: CO[C@H]1CCN(Cc2cc(-c3nc(-c4ccc5c(c4)CCNC5=O)c(N)nc3F)ccc2N2CCOCC2)C1] |
| I-157 | (S)-6-(3-amino-5-fluoro-6-(3- ((3-methoxypyrrolidin-1- yl)methyl)-4- morpholinophenyl)pyrazin-2- yl)-7-fluoro-3,4- dihydroisoquinolin-1(2H)-one | 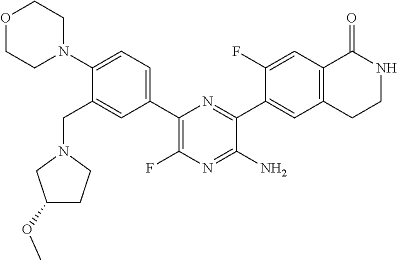 [SMILES: CO[C@H]1CCN(Cc2cc(-c3nc(-c4cc5c(cc4F)C(=O)NCC5)c(N)nc3F)ccc2N2CCOCC2)C1] |
| I-158 | (S)-6-(3-amino-5-fluoro-6-(3- ((3-methoxypyrrolidin-1- yl)methyl)-4- morpholinophenyl)pyrazin-2- yl)-8-fluoro-3,4- dihydroisoquinolin-1(2H)-one | 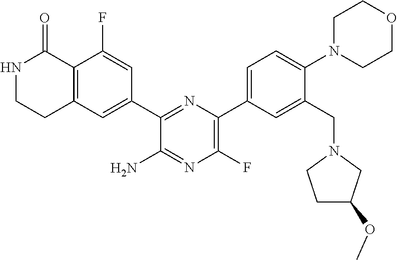 [SMILES: CO[C@H]1CCN(Cc2cc(-c3nc(-c4cc(F)c5c(c4)CCNC5=O)c(N)nc3F)ccc2N2CCOCC2)C1] |
| I-159 | 6-(3-amino-6-(4-(4-(4,4- difluorobutyl)piperazin-1- yl)phenyl)-5-fluoropyrazin-2-yl)- 8-fluoro-3-methylisoquinolin- 1(2H)-one | 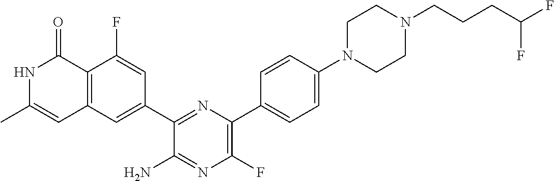 [SMILES: Cc1cc2cc(-c3nc(-c4ccc(N5CCN(CCCC(F)F)CC5)cc4)c(F)nc3N)cc(F)c2c(=O)[nH]1] |
| I-160 | 6-(3-amino-5-fluoro-6-(3-(((2- methoxyethyl)(methyl)amino) methyl)-4- morpholinophenyl)pyrazin-2- yl)-3,4-dihydroisoquinolin- 1(2H)-one | 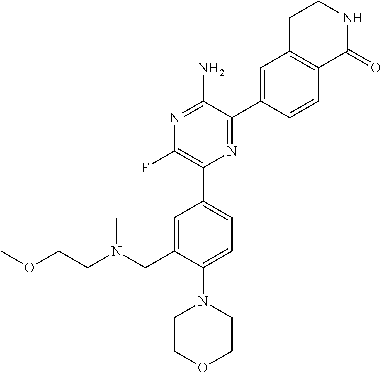 [SMILES: COCCN(C)Cc1cc(-c2nc(-c3ccc4c(c3)CCNC4=O)c(N)nc2F)ccc1N1CCOCC1] |
| I-161 | 6-(3-amino-5-fluoro-6-(3-(((2- methoxyethyl)(methyl)amino) methyl)-4- morpholinophenyl)pyrazin-2- yl)-7-fluoro-3,4- dihydroisoquinolin-1(2H)-one | 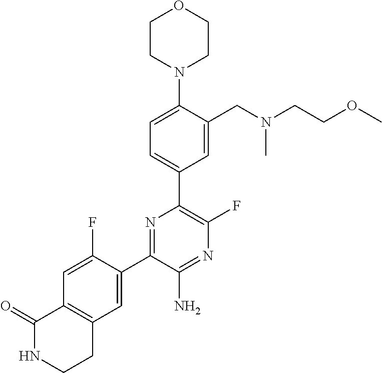 [SMILES: COCCN(C)Cc1cc(-c2nc(-c3cc4c(cc3F)C(=O)NCC4)c(N)nc2F)ccc1N1CCOCC1] |
| I-162 | 6-(3-amino-5-fluoro-6-(3-(((2- methoxyethyl)(methyl)amino) methyl)-4- morpholinophenyl)pyrazin-2- yl)-8-fluoro-3,4- dihydroisoquinolin-1(2H)-one | 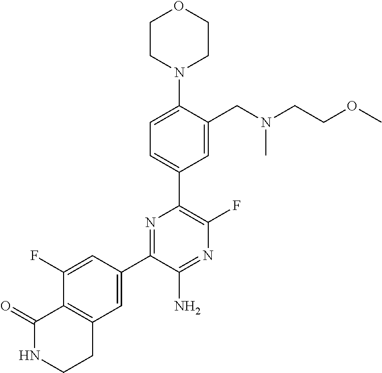 [SMILES: COCCN(C)Cc1cc(-c2nc(-c3cc(F)c4c(c3)CCNC4=O)c(N)nc2F)ccc1N1CCOCC1] |
| I-163 | 6-(3-amino-6-(3- ((ethyl(methyl)amino)methyl)- 4-(tetrahydro-2H-pyran-4- yl)phenyl)-5-fluoropyrazin-2-yl)- 3,4-dihydroisoquinolin-1(2H)- one | 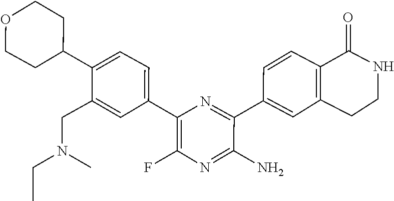 [SMILES: CCN(C)Cc1cc(-c2nc(-c3ccc4c(c3)CCNC4=O)c(N)nc2F)ccc1C1CCOCC1] |
| I-164 | 6-(3-amino-6-(3- ((ethyl(methyl)amino)methyl)- 4-(tetrahydro-2H-pyran-4- yl)phenyl)-5-fluoropyrazin-2-yl)- 7-fluoro-3,4-dihydroisoquinolin- 1(2H)-one | 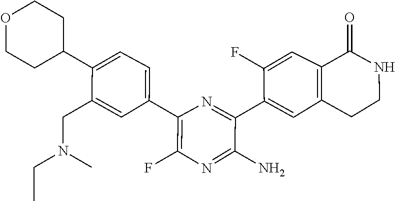 [SMILES: CCN(C)Cc1cc(-c2nc(-c3cc4c(cc3F)C(=O)NCC4)c(N)nc2F)ccc1C1CCOCC1] |
| I-165 | 6-(3-amino-6-(3- ((ethyl(methyl)amino)methyl)- 4-(tetrahydro-2H-pyran-4- yl)phenyl)-5-fluoropyrazin-2-yl)- 8-fluoro-3,4-dihydroisoquinolin- 1(2H)-one | 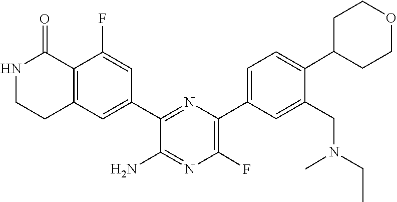 [SMILES: CCN(C)Cc1cc(-c2nc(-c3cc(F)c4c(c3)CCNC4=O)c(N)nc2F)ccc1C1CCOCC1] |
| I-166 | 6-(3-amino-5-fluoro-6-(4-(4- isopropylpiperazin-1- yl)phenyl)pyrazin-2-yl)-8-fluoro- 4-methylisoquinolin-1(2H)-one | 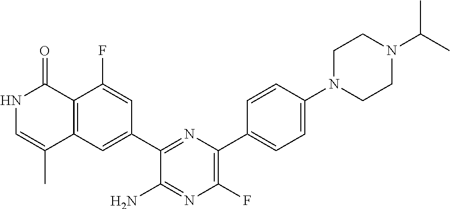 [SMILES: Cc1c[nH]c(=O)c2c(F)cc(-c3nc(-c4ccc(N5CCN(C(C)C)CC5)cc4)c(F)nc3N)cc12] |
| I-167 | 6-(3-amino-6-(3- ((dimethylamino)methyl)-4- (tetrahydro-2H-pyran-4- yl)phenyl)-5-fluoropyrazin-2-yl)- 8-fluoro-4-methylisoquinolin- 1(2H)-one | 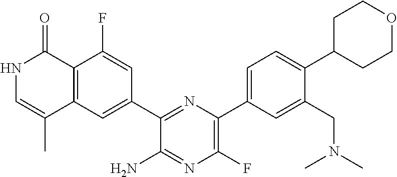 [SMILES: Cc1c[nH]c(=O)c2c(F)cc(-c3nc(-c4ccc(C5CCOCC5)c(CN(C)C)c4)c(F)nc3N)cc12] |
| I-168 | 6-(3-amino-5-fluoro-6-(4-(4- isopropylpiperazin-1- yl)phenyl)pyrazin-2-yl)-4,7- difluoro-3-methylisoquinolin- 1(2H)-one | 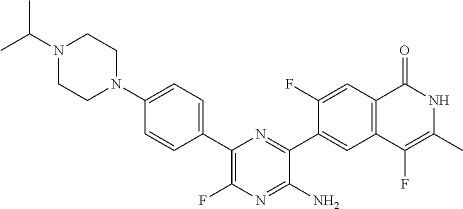 [SMILES: Cc1[nH]c(=O)c2cc(F)c(-c3nc(-c4ccc(N5CCN(C(C)C)CC5)cc4)c(F)nc3N)cc2c1F] |
| I-169 | 6-(3-amino-6-(3-(azetidin-1- ylmethyl)-4-(tetrahydro-2H- pyran-4-yl)phenyl)-5- fluoropyrazin-2-yl)-3,4- dihydroisoquinolin-1(2H)-one | 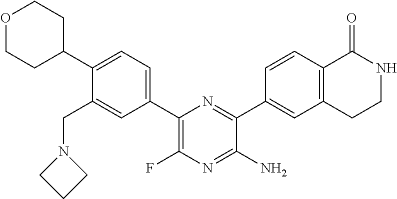 [SMILES: Nc1nc(F)c(-c2ccc(C3CCOCC3)c(CN3CCC3)c2)nc1-c1ccc2c(c1)CCNC2=O] |
| I-170 | 6-(3-amino-6-(3-(azetidin-1- ylmethyl)-4-(tetrahydro-2H- pyran-4-yl)phenyl)-5- fluoropyrazin-2-yl)-7-fluoro-3,4- dihydroisoquinolin-1(2H)-one | 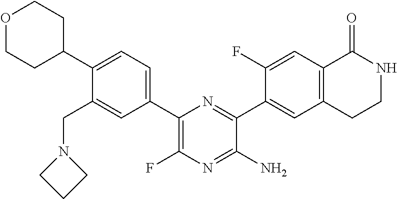 [SMILES: Nc1nc(F)c(-c2ccc(C3CCOCC3)c(CN3CCC3)c2)nc1-c1cc2c(cc1F)C(=O)NCC2] |
| I-171 | 6-(3-amino-6-(3-(azetidin-1- ylmethyl)-4-(tetrahydro-2H- pyran-4-yl)phenyl)-5- fluoropyrazin-2-yl)-8-fluoro-3,4- dihydroisoquinolin-1(2H)-one | 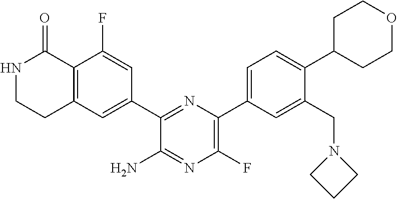 [SMILES: Nc1nc(F)c(-c2ccc(C3CCOCC3)c(CN3CCC3)c2)nc1-c1cc(F)c2c(c1)CCNC2=O] |
| I-172 | (R)-6-(3-amino-6-(3- ((dimethylamino)methyl)-4-(2- methylmorpholino)phenyl)-5- fluoropyrazin-2-yl)-3,4- dihydroisoquinolin-1(2H)-one | 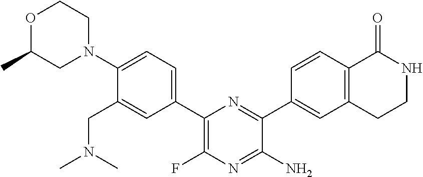 [SMILES: C[C@@H]1CN(c2ccc(-c3nc(-c4ccc5c(c4)CCNC5=O)c(N)nc3F)cc2CN(C)C)CCO1] |
| I-173 | (R)-6-(3-amino-6-(3- ((dimethylamino)methyl)-4-(2- methylmorpholino)phenyl)-5- fluoropyrazin-2-yl)-7-fluoro-3,4- dihydroisoquinolin-1(2H)-one | 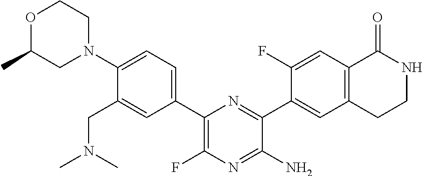 [SMILES: C[C@@H]1CN(c2ccc(-c3nc(-c4cc5c(cc4F)C(=O)NCC5)c(N)nc3F)cc2CN(C)C)CCO1] |
| I-174 | 6-(3-amino-5-fluoro-6-(3-(((2- methoxyethyl)(methyl)amino) methyl)-4-(tetrahydro-2H-pyran- 4-yl)phenyl)pyrazin-2-yl)-3,4- dihydroisoquinolin-1(2H)-one | 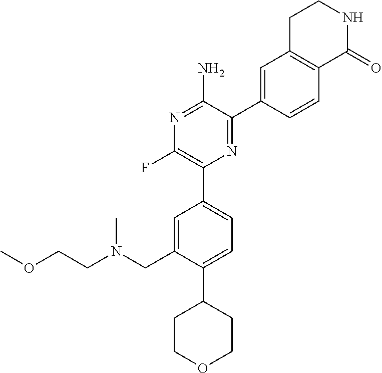 [SMILES: COCCN(C)Cc1cc(-c2nc(-c3ccc4c(c3)CCNC4=O)c(N)nc2F)ccc1C1CCOCC1] |
| I-175 | 6-(3-amino-5-fluoro-6-(3-(((2- methoxyethyl)(methyl)amino) methyl)-4-(tetrahydro-2H-pyran- 4-yl)phenyl)pyrazin-2-yl)-7- fluoro-3,4-dihydroisoquinolin- 1(2H)-one | 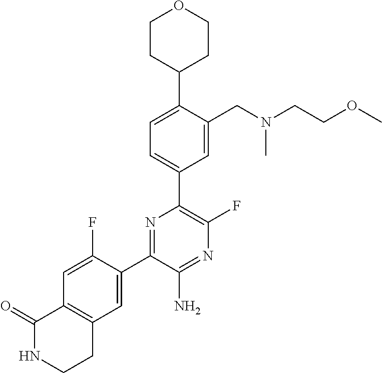 [SMILES: COCCN(C)Cc1cc(-c2nc(-c3cc4c(cc3F)C(=O)NCC4)c(N)nc2F)ccc1C1CCOCC1] |
| I-176 | 6-(3-amino-5-fluoro-6-(3-(((2- methoxyethyl)(methyl)amino) methyl)-4-(tetrahydro-2H-pyran- 4-yl)phenyl)pyrazin-2-yl)-8- fluoro-3,4-dihydroisoquinolin- 1(2H)-one | 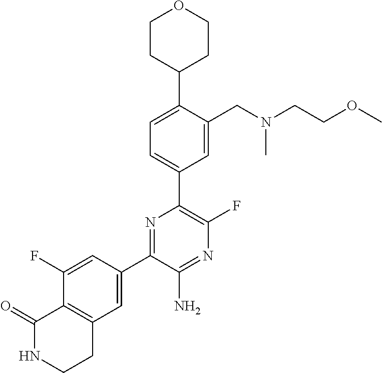 [SMILES: COCCN(C)Cc1cc(-c2nc(-c3cc(F)c4c(c3)CCNC4=O)c(N)nc2F)ccc1C1CCOCC1] |
| I-177 | (R)-6-(3-amino-6-(3- ((dimethylamino)methyl)-4-(2- methylmorpholino)phenyl)-5- fluoropyrazin-2-yl)-8-fluoro-3,4- dihydroisoquinolin-1(2H)-one | 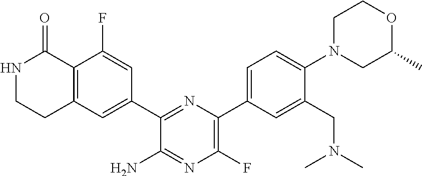 [SMILES: C[C@@H]1CN(c2ccc(-c3nc(-c4cc(F)c5c(c4)CCNC5=O)c(N)nc3F)cc2CN(C)C)CCO1] |
| I-178 | 6-(3-amino-6-(3- ((dimethylamino)methyl)-4- ((tetrahydro-2H-pyran-4- yl)methoxy)phenyl)-5- fluoropyrazin-2-yl)-3,4- dihydroisoquinolin-1(2H)-one | 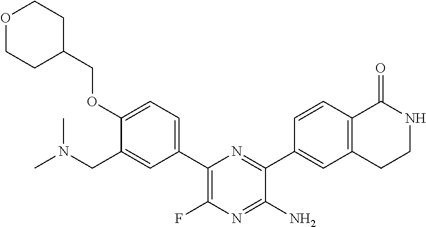 [SMILES: CN(C)Cc1cc(-c2nc(-c3ccc4c(c3)CCNC4=O)c(N)nc2F)ccc1OCC1CCOCC1] |
| I-179 | 6-(3-amino-6-(3- ((dimethylamino)methyl)-4- ((tetrahydro-2H-pyran-4- yl)methoxy)phenyl)-5- fluoropyrazin-2-yl)-7-fluoro-3,4- dihydroisoquinolin-1(2H)-one | 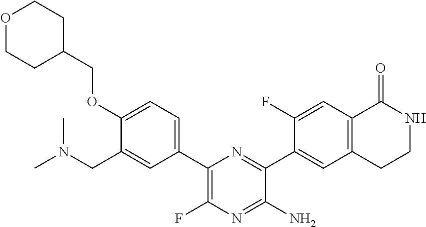 [SMILES: CN(C)Cc1cc(-c2nc(-c3cc4c(cc3F)C(=O)NCC4)c(N)nc2F)ccc1OCC1CCOCC1] |
| I-180 | 6-(3-amino-6-(3- ((dimethylamino)methyl)-4- ((tetrahydro-2H-pyran-4- yl)methoxy)phenyl)-5- fluoropyrazin-2-yl)-8-fluoro-3,4- dihydroisoquinolin-1(2H)-one | 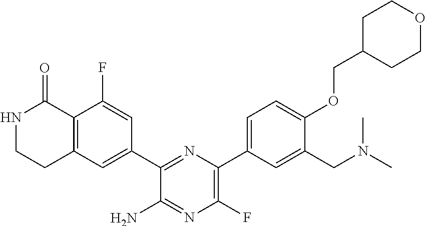 [SMILES: CN(C)Cc1cc(-c2nc(-c3cc(F)c4c(c3)CCNC4=O)c(N)nc2F)ccc1OCC1CCOCC1] |
| I-181 | 6-(3-amino-5-fluoro-6-(4- (tetrahydro-2H-pyran-4- yl)phenyl)pyrazin-2-yl)-4- methylisoquinolin-1(2H)-one | 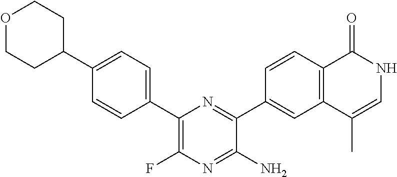 [SMILES: Cc1c[nH]c(=O)c2ccc(-c3nc(-c4ccc(C5CCOCC5)cc4)c(F)nc3N)cc12] |
| I-182 | 6-(3-amino-5-fluoro-6-(4-(4- isopropylpiperazin-1- yl)phenyl)pyrazin-2-yl)-7-fluoro- 3-methylisoquinolin-1(2H)-one | 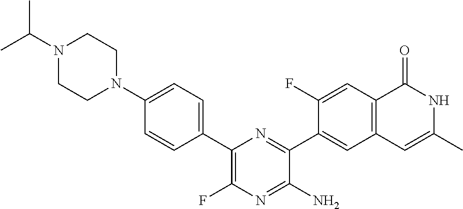 [SMILES: Cc1cc2cc(-c3nc(-c4ccc(N5CCN(C(C)C)CC5)cc4)c(F)nc3N)c(F)cc2c(=O)[nH]1] |
| I-183 | 6-(3-amino-6-(3- ((dimethylamino)methyl)-4- morpholinophenyl)-5- fluoropyrazin-2-yl)-7-fluoro-3- methylisoquinolin-1(2H)-one | 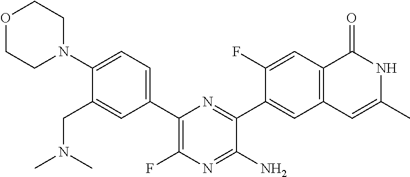 [SMILES: Cc1cc2cc(-c3nc(-c4ccc(N5CCOCC5)c(CN(C)C)c4)c(F)nc3N)c(F)cc2c(=O)[nH]1] |
| I-184 | 6-(3-amino-6-(3- ((dimethylamino)methyl)-4- (tetrahydro-2H-pyran-4- yl)phenyl)-5-fluoropyrazin-2-yl)- 7-fluoro-3-methylisoquinolin- 1(2H)-one | 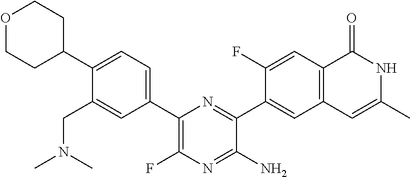 [SMILES: Cc1cc2cc(-c3nc(-c4ccc(C5CCOCC5)c(CN(C)C)c4)c(F)nc3N)c(F)cc2c(=O)[nH]1] |
| I-185 | 6-(3-amino-5-fluoro-6-(4- morpholino-3-(piperidin-1- ylmethyl)phenyl)pyrazin-2-yl)- 3,4-dihydroisoquinolin-1(2H)- one | 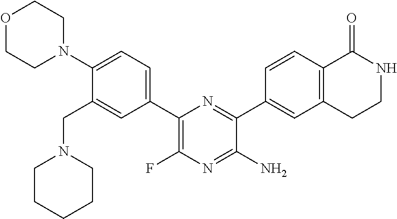 [SMILES: Nc1nc(F)c(-c2ccc(N3CCOCC3)c(CN3CCCCC3)c2)nc1-c1ccc2c(c1)CCNC2=O] |
| I-186 | 6-(3-amino-5-fluoro-6-(4- morpholino-3-(piperidin-1- ylmethyl)phenyl)pyrazin-2-yl)- 7-fluoro-3,4-dihydroisoquinolin- 1(2H)-one | 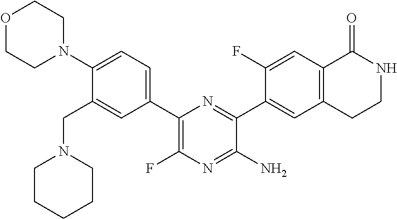 [SMILES: Nc1nc(F)c(-c2ccc(N3CCOCC3)c(CN3CCCCC3)c2)nc1-c1cc2c(cc1F)C(=O)NCC2] |
| I-187 | 6-(3-amino-5-fluoro-6-(4- morpholino-3-(piperidin-1- ylmethyl)phenyl)pyrazin-2-yl)- 8-fluoro-3,4-dihydroisoquinolin- 1(2H)-one | 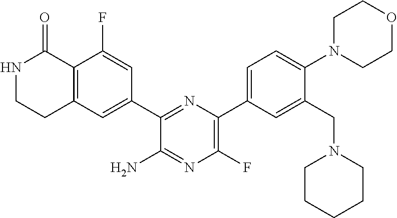 [SMILES: Nc1nc(F)c(-c2ccc(N3CCOCC3)c(CN3CCCCC3)c2)nc1-c1cc(F)c2c(c1)CCNC2=O] |
| I-188 | 6-(3-amino-5-fluoro-6-(3-((3- methoxyazetidin-1-yl)methyl)- 4-morpholinophenyl)pyrazin-2- yl)-3,4-dihydroisoquinolin- 1(2H)-one | 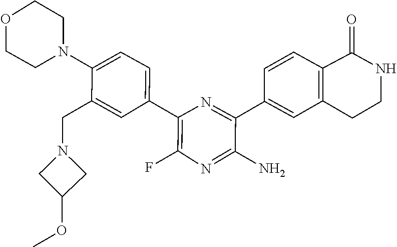 [SMILES: COC1CN(Cc2cc(-c3nc(-c4ccc5c(c4)CCNC5=O)c(N)nc3F)ccc2N2CCOCC2)C1] |
| I-189 | 6-(3-amino-5-fluoro-6-(3-((3- methoxyazetidin-1-yl)methyl)- 4-morpholinophenyl)pyrazin-2- yl)-7-fluoro-3,4- dihydroisoquinolin-1(2H)-one | 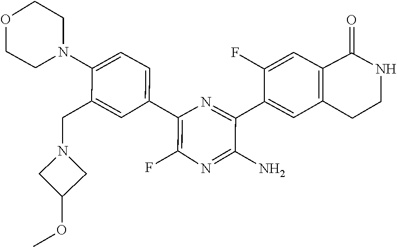 [SMILES: COC1CN(Cc2cc(-c3nc(-c4cc5c(cc4F)C(=O)NCC5)c(N)nc3F)ccc2N2CCOCC2)C1] |
| I-190 | 6-(3-amino-5-fluoro-6-(3-((3- methoxyazetidin-1-yl)methyl)- 4-(tetrahydro-2H-pyran-4- yl)phenyl)pyrazin-2-yl)-7-fluoro- 3,4-dihydroisoquinolin-1(2H)- one | 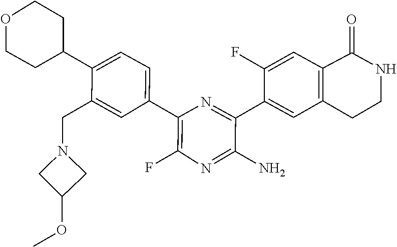 [SMILES: COC1CN(Cc2cc(-c3nc(-c4cc5c(cc4F)C(=O)NCC5)c(N)nc3F)ccc2C2CCOCC2)C1] |
| I-191 | 6-(3-amino-5-fluoro-6-(3-((3- methoxyazetidin-1-yl)methyl)- 4-(tetrahydro-2H-pyran-4- yl)phenyl)pyrazin-2-yl)-8-fluoro- 3,4-dihydroisoquinolin-1(2H)- one | 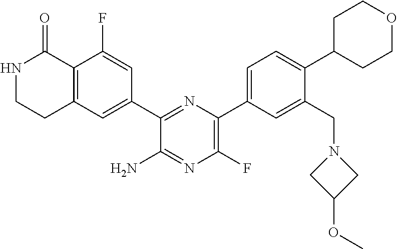 [SMILES: COC1CN(Cc2cc(-c3nc(-c4cc(F)c5c(c4)CCNC5=O)c(N)nc3F)ccc2C2CCOCC2)C1] |
| I-192 | 6-(3-amino-5-fluoro-6-(3-((3- methoxyazetidin-1-yl)methyl)- 4-morpholinophenyl)pyrazin-2- yl)-8-fluoro-3,4- dihydroisoquinolin-1(2H)-one | 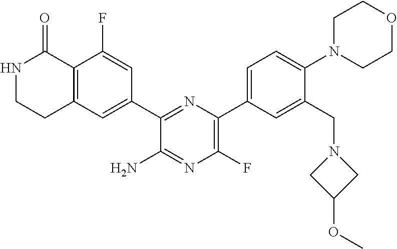 [SMILES: COC1CN(Cc2cc(-c3nc(-c4cc(F)c5c(c4)CCNC5=O)c(N)nc3F)ccc2N2CCOCC2)C1] |
| I-193 | 6-(3-amino-5-fluoro-6-(3-((3- methoxyazetidin-1-yl)methyl)- 4-(tetrahydro-2H-pyran-4- yl)phenyl)pyrazin-2-yl)-3,4- dihydroisoquinolin-1(2H)-one | 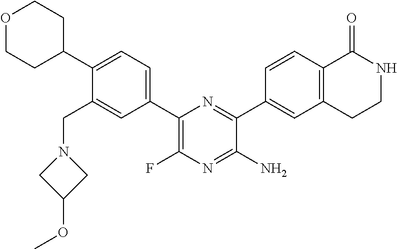 [SMILES: COC1CN(Cc2cc(-c3nc(-c4ccc5c(c4)CCNC5=O)c(N)nc3F)ccc2C2CCOCC2)C1] |
| I-194 | 7-(3-amino-6-(3- ((dimethylamino)methyl)-4- ((2R,6S)-2,6- dimethylmorpholino)phenyl)-5- fluoropyrazin-2-yl)-2- methylquinazolin-4(3H)-one | 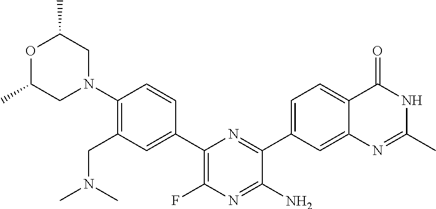 |
| I-195 | 6-(3-amino-6-(4-((1S,5R)-3- cyclobutyl-3- azabicyclo[3.1.0]hexan-1- yl)phenyl)-5-fluoropyrazin-2-yl)- 4,4,8-trifluoro-3-methyl-3,4- dihydroisoquinolin-1(2H)-one | 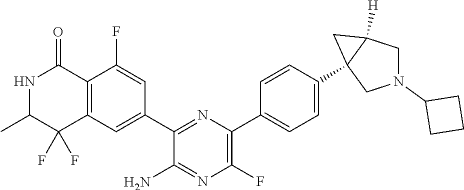 [SMILES: CC1NC(=O)c2c(F)cc(-c3nc(-c4ccc([C@]56C[C@H]5CN(C5CCC5)C6)cc4)c(F)nc3N)cc2C1(F)F] |
| I-196 | 7-(3-amino-6-(3- ((dimethylamino)methyl)-4- ((2R,6S)-2,6- dimethylmorpholino)phenyl)-5- fluoropyrazin-2-yl)-5-fluoro-2- methylquinazolin-4(3H)-one | 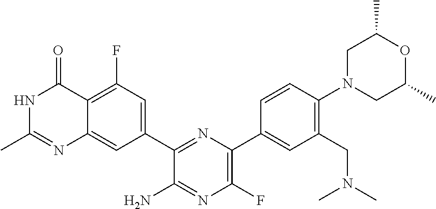 |
| I-197 | 6-(3-amino-6-(3- ((dimethylamino)methyl)-4- ((2R,6S)-2,6- dimethylmorpholino)phenyl)-5- fluoropyrazin-2-yl)-8-fluoro-3,4- dihydroisoquinolin-1(2H)-one | 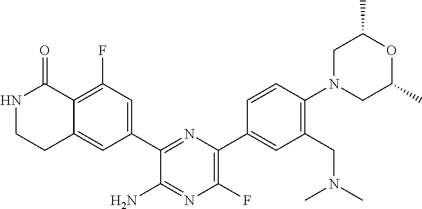 [SMILES: C[C@@H]1CN(c2ccc(-c3nc(-c4cc(F)c5c(c4)CCNC5=O)c(N)nc3F)cc2CN(C)C)C[C@H](C)O1] |
| I-198 | 6-(3-amino-6-(3- ((dimethylamino)methyl)-4- ((2R,6S)-2,6- dimethylmorpholino)phenyl)-5- fluoropyrazin-2-yl)-3,4- dihydroisoquinolin-1(2H)-one | 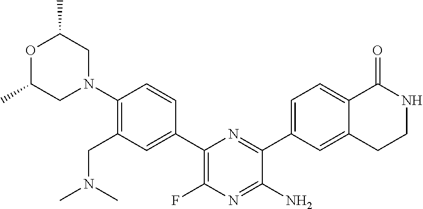 [SMILES: C[C@@H]1CN(c2ccc(-c3nc(-c4ccc5c(c4)CCNC5=O)c(N)nc3F)cc2CN(C)C)C[C@H](C)O1] |
| I-1996 | 6-(3-amino-6-(3-(azetidin-1- ylmethyl)-4-morpholinophenyl)- 5-fluoropyrazin-2-yl)-4- fluoroisoquinolin-1(2H)-one | 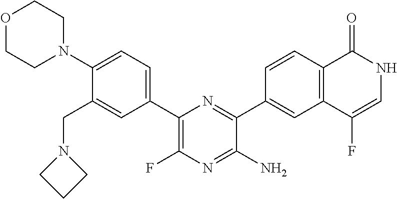 [SMILES: Nc1nc(F)c(-c2ccc(N3CCOCC3)c(CN3CCC3)c2)nc1-c1ccc2c(=O)[nH]cc(F)c2c1] |
| I-200 | 6-(3-amino-5-fluoro-6-(4- morpholino-3-(pyrrolidin-1- ylmethyl)phenyl)pyrazin-2-yl)- 4-fluoroisoquinolin-1(2H)-one | 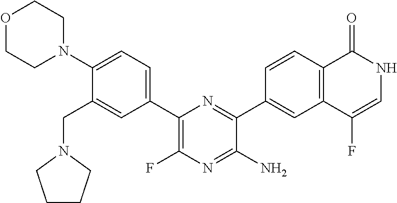 [SMILES: Nc1nc(F)c(-c2ccc(N3CCOCC3)c(CN3CCCC3)c2)nc1-c1ccc2c(=O)[nH]cc(F)c2c1] |
| I-201 | 6-(3-amino-5-fluoro-6-(5- morpholinopyridin-2-yl)pyrazin- 2-yl)-3,4-dihydroisoquinolin- 1(2H)-one | 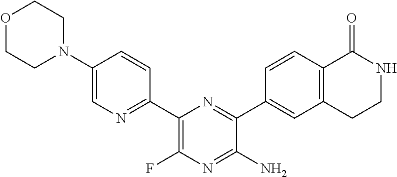 [SMILES: Nc1nc(F)c(-c2ccc(N3CCOCC3)cn2)nc1-c1ccc2c(c1)CCNC2=O] |
| I-202 | 6-(3-amino-6-(3-(azetidin-1- ylmethyl)-4-morpholinophenyl)- 5-fluoropyrazin-2-yl)-4- methylisoquinolin-1(2H)-one | 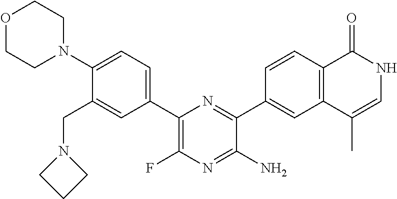 [SMILES: Cc1c[nH]c(=O)c2ccc(-c3nc(-c4ccc(N5CCOCC5)c(CN5CCC5)c4)c(F)nc3N)cc12] |
| I-203 | 6-(3-amino-5-fluoro-6-(4- morpholino-3-(pyrrolidin-1- ylmethyl)phenyl)pyrazin-2-yl)- 4-methylisoquinolin-1(2H)-one | 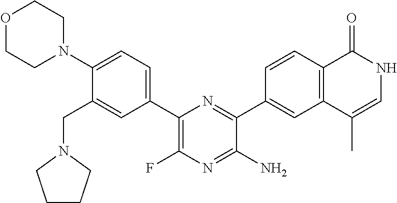 [SMILES: Cc1c[nH]c(=O)c2ccc(-c3nc(-c4ccc(N5CCOCC5)c(CN5CCCC5)c4)c(F)nc3N)cc12] |
| I-204 | 6-(3-amino-6-(3-(azetidin-1- ylmethyl)-4-(tetrahydro-2H- pyran-4-yl)phenyl)-5- fluoropyrazin-2-yl)-4- fluoroisoquinolin-1(2H)-one | 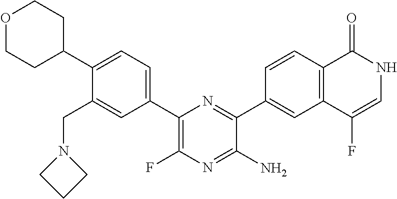 [SMILES: Nc1nc(F)c(-c2ccc(C3CCOCC3)c(CN3CCC3)c2)nc1-c1ccc2c(=O)[nH]cc(F)c2c1] |
| I-205 | 6-(3-amino-5-fluoro-6-(3- (pyrrolidin-1-ylmethyl)-4- (tetrahydro-2H-pyran-4- yl)phenyl)pyrazin-2-yl)-4- methylisoquinolin-1(2H)-one | 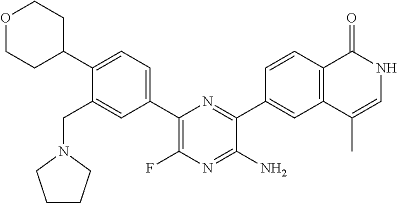 [SMILES: Cc1c[nH]c(=O)c2ccc(-c3nc(-c4ccc(C5CCOCC5)c(CN5CCCC5)c4)c(F)nc3N)cc12] |
| I-206 | 6-(3-amino-6-(3-(azetidin-1- ylmethyl)-4-(tetrahydro-2H- pyran-4-yl)phenyl)-5- fluoropyrazin-2-yl)-4- methylisoquinolin-1(2H)-one | 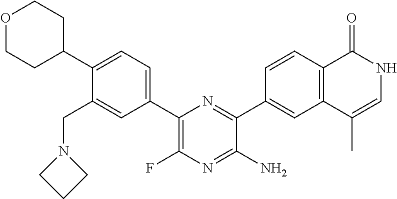 [SMILES: Cc1c[nH]c(=O)c2ccc(-c3nc(-c4ccc(C5CCOCC5)c(CN5CCC5)c4)c(F)nc3N)cc12] |
| I-207 | 6-(3-amino-5-fluoro-6-(3- (pyrrolidin-1-ylmethyl)-4- (tetrahydro-2H-pyran-4- yl)phenyl)pyrazin-2-yl)-4- fluoroisoquinolin-1(2H)-one | 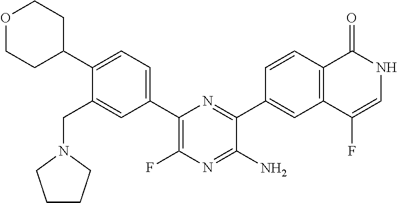 [SMILES: Nc1nc(F)c(-c2ccc(C3CCOCC3)c(CN3CCCC3)c2)nc1-c1ccc2c(=O)[nH]cc(F)c2c1] |
| I-208 | 6-(3-amino-6-(3-(azetidin-1- ylmethyl)-4-((tetrahydro-2H- pyran-4-yl)oxy)phenyl)-5- fluoropyrazin-2-yl)-3,4- dihydroisoquinolin-1(2H)-one | 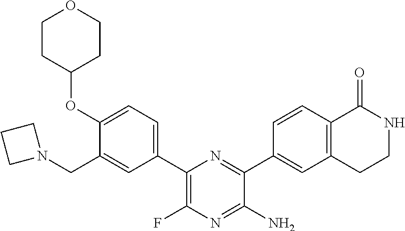 [SMILES: Nc1nc(F)c(-c2ccc(OC3CCOCC3)c(CN3CCC3)c2)nc1-c1ccc2c(c1)CCNC2=O] |
| I-209 | 6-(3-amino-6-(3-(azetidin-1- ylmethyl)-4-((tetrahydro-2H- pyran-4-yl)oxy)phenyl)-5- fluoropyrazin-2-yl)-7-fluoro-3,4- dihydroisoquinolin-1(2H)-one | 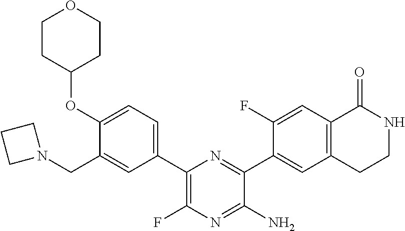 [SMILES: Nc1nc(F)c(-c2ccc(OC3CCOCC3)c(CN3CCC3)c2)nc1-c1cc2c(cc1F)C(=O)NCC2] |
| I-210 | 6-(3-amino-6-(3-(azetidin-1- ylmethyl)-4-((tetrahydro-2H- pyran-4-yl)oxy)phenyl)-5- fluoropyrazin-2-yl)-8-fluoro-3,4- dihydroisoquinolin-1(2H)-one | 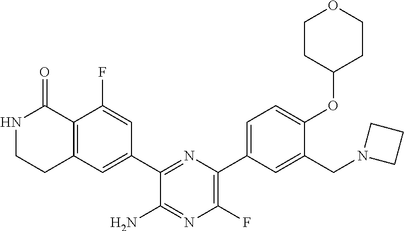 [SMILES: Nc1nc(F)c(-c2ccc(OC3CCOCC3)c(CN3CCC3)c2)nc1-c1cc(F)c2c(c1)CCNC2=O] |
| I-211 | (S)-6-(3-amino-6-(3- ((dimethylamino)methyl)-4-(2- methylmorpholino)phenyl)-5- fluoropyrazin-2-yl)-3,4- dihydroisoquinolin-1(2H)-one | 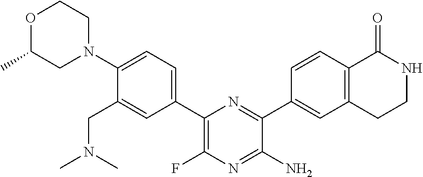 [SMILES: C[C@H]1CN(c2ccc(-c3nc(-c4ccc5c(c4)CCNC5=O)c(N)nc3F)cc2CN(C)C)CCO1] |
| I-212 | (S)-6-(3-amino-6-(3- ((dimethylamino)methyl)-4-(2- methylmorpholino)phenyl)-5- fluoropyrazin-2-yl)-7-fluoro-3,4- dihydroisoquinolin-1(2H)-one | 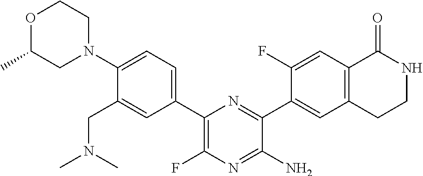 [SMILES: C[C@H]1CN(c2ccc(-c3nc(-c4cc5c(cc4F)C(=O)NCC5)c(N)nc3F)cc2CN(C)C)CCO1] |
| I-213 | (S)-6-(3-amino-6-(3- ((dimethylamino)methyl)-4-(2- methylmorpholino)phenyl)-5- fluoropyrazin-2-yl)-8-fluoro-3,4- dihydroisoquinolin-1(2H)-one | 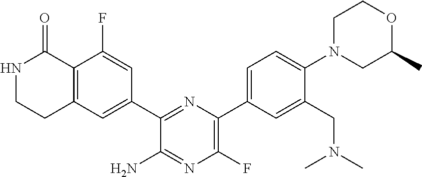 [SMILES: C[C@H]1CN(c2ccc(-c3nc(-c4cc(F)c5c(c4)CCNC5=O)c(N)nc3F)cc2CN(C)C)CCO1] |
| I-214 | (S)-6-(3-amino-6-(3-(azetidin- 1-ylmethyl)-4-(2- methylmorpholino)phenyl)-5- fluoropyrazin-2-yl)-3,4- dihydroisoquinolin-1(2H)-one | 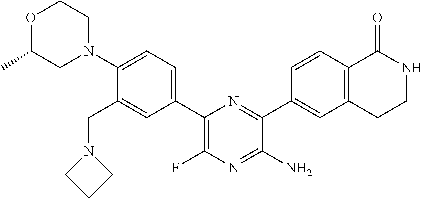 [SMILES: C[C@H]1CN(c2ccc(-c3nc(-c4ccc5c(c4)CCNC5=O)c(N)nc3F)cc2CN2CCC2)CCO1] |
| I-215 | (S)-6-(3-amino-6-(3-(azetidin- 1-ylmethyl)-4-(2- methylmorpholino)phenyl)-5- fluoropyrazin-2-yl)-7-fluoro-3,4- dihydroisoquinolin-1(2H)-one | 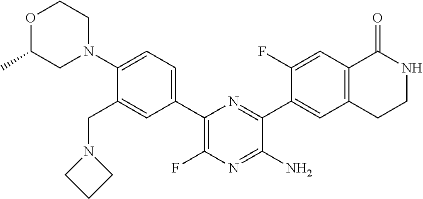 [SMILES: C[C@H]1CN(c2ccc(-c3nc(-c4cc5c(cc4F)C(=O)NCC5)c(N)nc3F)cc2CN2CCC2)CCO1] |
| I-216 | (S)-6-(3-amino-6-(3-(azetidin- 1-ylmethyl)-4-(2- methylmorpholino)phenyl)-5- fluoropyrazin-2-yl)-8-fluoro-3,4- dihydroisoquinolin-1(2H)-one | 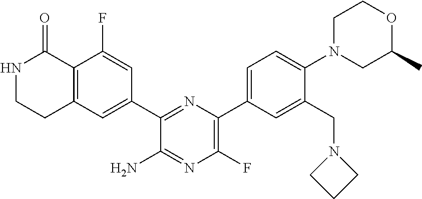 [SMILES: C[C@H]1CN(c2ccc(-c3nc(-c4cc(F)c5c(c4)CCNC5=O)c(N)nc3F)cc2CN2CCC2)CCO1] |
| I-217 | (R)-7-(3-amino-5-fluoro-6-(3- ((2-methylpyrrolidin-1- yl)methyl)-4-(tetrahydro-2H- pyran-4-yl)phenyl)pyrazin-2-yl)- 5-fluoro-2-methylquinazolin- 4(3H)-one | 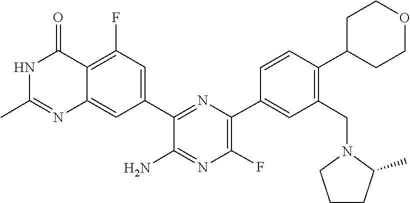 |
| I-218 | (R)-6-(3-amino-5-fluoro-6-(3- ((2-methylpyrrolidin-1- yl)methyl)-4-(tetrahydro-2H- pyran-4-yl)phenyl)pyrazin-2-yl)- 8-fluoro-3,4-dihydroisoquinolin- 1(2H)-one | 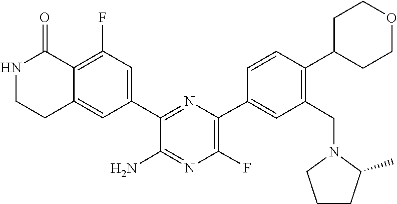 [SMILES: C[C@@H]1CCCN1Cc1cc(-c2nc(-c3cc(F)c4c(c3)CCNC4=O)c(N)nc2F)ccc1C1CCOCC1] |
| I-219 | (S)-7-(3-amino-5-fluoro-6-(3- ((2-methylpyrrolidin-1- yl)methyl)-4-(tetrahydro-2H- pyran-4-yl)phenyl)pyrazin-2-yl)- 5-fluoro-2-methylquinazolin- 4(3H)-one | 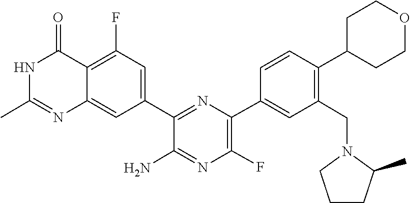 |
| I-220 | (S)-6-(3-amino-5-fluoro-6-(3- ((2-methylpyrrolidin-1- yl)methyl)-4-(tetrahydro-2H- pyran-4-yl)phenyl)pyrazin-2-yl)- 8-fluoro-3,4-dihydroisoquinolin- 1(2H)-one | 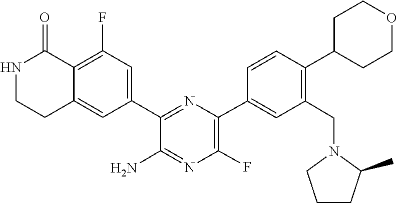 [SMILES: C[C@H]1CCCN1Cc1cc(-c2nc(-c3cc(F)c4c(c3)CCNC4=O)c(N)nc2F)ccc1C1CCOCC1] |
| I-221 | 6-(3-amino-5-fluoro-6-(3- methyl-4-(tetrahydro-2H-pyran- 4-yl)phenyl)pyrazin-2-yl)-8- fluoro-3,4-dihydroisoquinolin- 1(2H)-one | 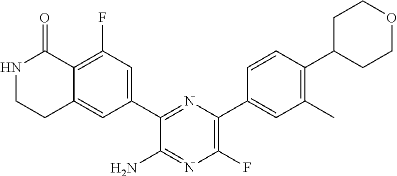 [SMILES: Cc1cc(-c2nc(-c3cc(F)c4c(c3)CCNC4=O)c(N)nc2F)ccc1C1CCOCC1] |
| I-222 | 6-(3-amino-5-fluoro-6-(3- methyl-4-(tetrahydro-2H-pyran- 4-yl)phenyl)pyrazin-2-yl)-7- fluoro-3,4-dihydroisoquinolin- 1(2H)-one | 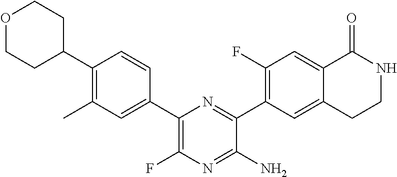 [SMILES: Cc1cc(-c2nc(-c3cc4c(cc3F)C(=O)NCC4)c(N)nc2F)ccc1C1CCOCC1] |
| I-223 | 6-(3-amino-6-(3- ((dimethylamino)methyl)-4- (tetrahydro-2H-pyran-4- yl)phenyl)-5-fluoropyrazin-2-yl)- 4,4-difluoro-3,4- dihydroisoquinolin-1(2H)-one | 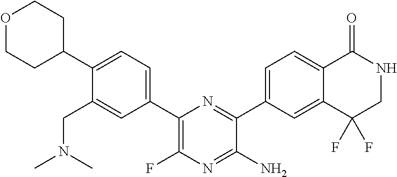 [SMILES: CN(C)Cc1cc(-c2nc(-c3ccc4c(c3)C(F)(F)CNC4=O)c(N)nc2F)ccc1C1CCOCC1] |
| I-224 | 6-(3-amino-6-(3- ((ethyl(methyl)amino)methyl)- 4-(tetrahydro-2H-pyran-4- yl)phenyl)-5-fluoropyrazin-2-yl)- 4,4-difluoro-3,4- dihydroisoquinolin-1(2H)-one | 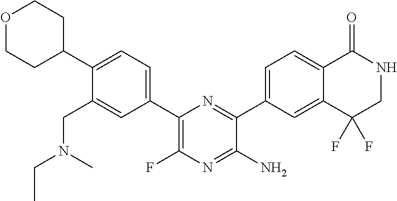 [SMILES: CCN(C)Cc1cc(-c2nc(-c3ccc4c(c3)C(F)(F)CNC4=O)c(N)nc2F)ccc1C1CCOCC1] |
| I-225 | 6-(3-amino-6-(4-(3,6-dihydro- 2H-pyran-4-yl)-3- ((ethyl(methyl)amino)methyl) phenyl)-5-fluoropyrazin-2-yl)-4,4- difluoro-3,4-dihydroisoquinolin- 1(2H)-one | 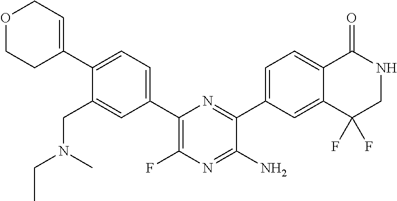 [SMILES: CCN(C)Cc1cc(-c2nc(-c3ccc4c(c3)C(F)(F)CNC4=O)c(N)nc2F)ccc1C1=CCOCC1] |
| I-226 | (R)-6-(3-amino-5-fluoro-6-(3- ((2-methylpyrrolidin-1- yl)methyl)-4-(tetrahydro-2H- pyran-4-yl)phenyl)pyrazin-2-yl)- 4-fluoroisoquinolin-1(2H)-one | 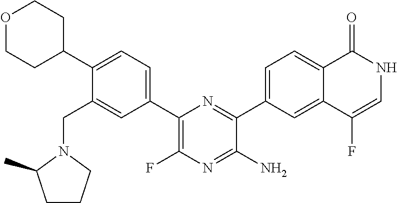 [SMILES: C[C@@H]1CCCN1Cc1cc(-c2nc(-c3ccc4c(=O)[nH]cc(F)c4c3)c(N)nc2F)ccc1C1CCOCC1] |
| I-227 | (R)-6-(3-amino-5-fluoro-6-(3- ((2-methylpyrrolidin-1- yl)methyl)-4-(tetrahydro-2H- pyran-4-yl)phenyl)pyrazin-2-yl)- 4-chloroisoquinolin-1(2H)-one | 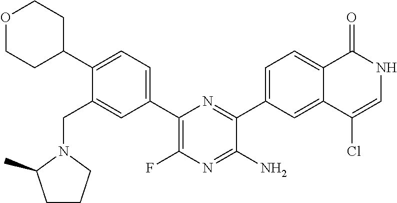 [SMILES: C[C@@H]1CCCN1Cc1cc(-c2nc(-c3ccc4c(=O)[nH]cc(Cl)c4c3)c(N)nc2F)ccc1C1CCOCC1] |
| I-228 | (S)-6-(3-amino-5-fluoro-6-(3- ((2-methylpyrrolidin-1- yl)methyl)-4-(tetrahydro-2H- pyran-4-yl)phenyl)pyrazin-2-yl)- 4-fluoroisoquinolin-1(2H)-one | 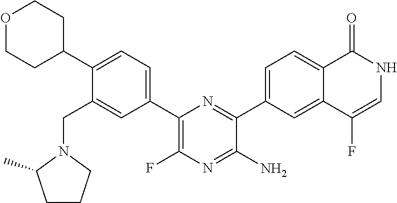 [SMILES: C[C@H]1CCCN1Cc1cc(-c2nc(-c3ccc4c(=O)[nH]cc(F)c4c3)c(N)nc2F)ccc1C1CCOCC1] |
| I-229 | (S)-6-(3-amino-5-fluoro-6-(3- ((2-methylpyrrolidin-1- yl)methyl)-4-(tetrahydro-2H- pyran-4-yl)phenyl)pyrazin-2-yl)- 4-chloroisoquinolin-1(2H)-one | 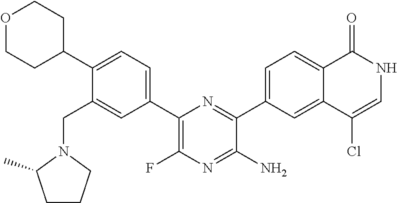 [SMILES: C[C@H]1CCCN1Cc1cc(-c2nc(-c3ccc4c(=O)[nH]cc(Cl)c4c3)c(N)nc2F)ccc1C1CCOCC1] |
| I-230 | 6-(3-amino-6-(3- ((dimethylamino)methyl)-4- ((2R,6S)-2,6- dimethylmorpholino)phenyl)-5- fluoropyrazin-2-yl)-4- fluoroisoquinolin-1(2H)-one | 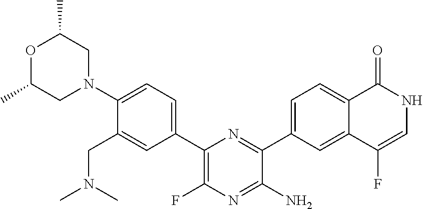 [SMILES: C[C@@H]1CN(c2ccc(-c3nc(-c4ccc5c(=O)[nH]cc(F)c5c4)c(N)nc3F)cc2CN(C)C)C[C@H](C)O1] |
| I-231 | 6-(3-amino-6-(3- ((dimethylamino)methyl)-4- ((2R,6S)-2,6- dimethylmorpholino)phenyl)-5- fluoropyrazin-2-yl)-4- chloroisoquinolin-1(2H)-one | 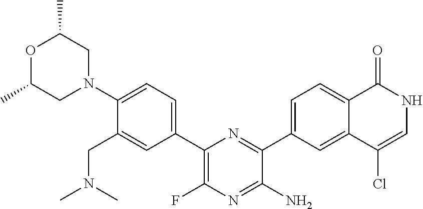 [SMILES: C[C@@H]1CN(c2ccc(-c3nc(-c4ccc5c(=O)[nH]cc(Cl)c5c4)c(N)nc3F)cc2CN(C)C)C[C@H](C)O1] |
| I-232 | (R)-6-(3-amino-5-fluoro-6-(3- ((2-methylpyrrolidin-1- yl)methyl)-4-(tetrahydro-2H- pyran-4-yl)phenyl)pyrazin-2-yl)- 4,4-difluoro-3,4- dihydroisoquinolin-1(2H)-one | 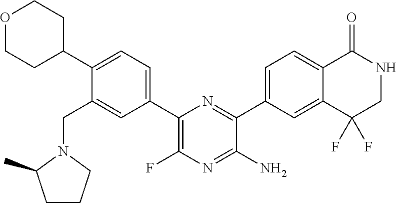 [SMILES: C[C@@H]1CCCN1Cc1cc(-c2nc(-c3ccc4c(c3)C(F)(F)CNC4=O)c(N)nc2F)ccc1C1CCOCC1] |
| I-233 | (S)-6-(3-amino-5-fluoro-6-(3- ((2-methylpyrrolidin-1- yl)methyl)-4-(tetrahydro-2H- pyran-4-yl)phenyl)pyrazin-2-yl)- 4,4-difluoro-3,4- dihydroisoquinolin-1(2H)-one | 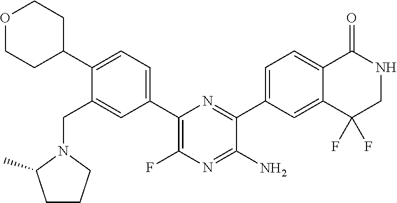 [SMILES: C[C@H]1CCCN1Cc1cc(-c2nc(-c3ccc4c(c3)C(F)(F)CNC4=O)c(N)nc2F)ccc1C1CCOCC1] |
| I-234 | 6-(3-amino-5-fluoro-6-(4-(4- isopropylpiperazin-1- yl)phenyl)pyrazin-2-yl)-7-fluoro- 4-methylisoquinolin-1(2H)-one | 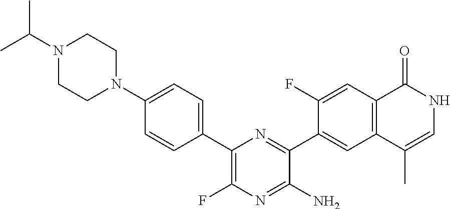 [SMILES: Cc1c[nH]c(=O)c2cc(F)c(-c3nc(-c4ccc(N5CCN(C(C)C)CC5)cc4)c(F)nc3N)cc12] |
| I-235 | 6-(3-amino-6-(3- ((dimethylamino)methyl)-4- (tetrahydro-2H-pyran-4- yl)phenyl)-5-fluoropyrazin-2-yl)- 7-fluoro-4-methylisoquinolin- 1(2H)-one | 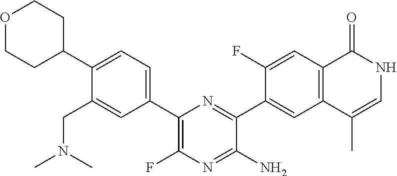 [SMILES: Cc1c[nH]c(=O)c2cc(F)c(-c3nc(-c4ccc(C5CCOCC5)c(CN(C)C)c4)c(F)nc3N)cc12] |
| I-236 | 6-(3-amino-6-(5- ((dimethylamino)methyl)-6- morpholinopyridin-3-yl)-5- fluoropyrazin-2-yl)-4- methylisoquinolin-1(2H)-one | 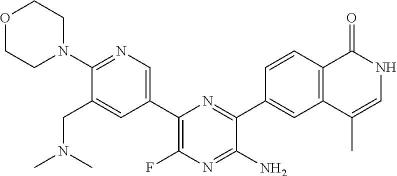 [SMILES: Cc1c[nH]c(=O)c2ccc(-c3nc(-c4cnc(N5CCOCC5)c(CN(C)C)c4)c(F)nc3N)cc12] |
| I-237 | 6-(3-amino-6-(5- ((dimethylamino)methyl)-6- morpholinopyridin-3-yl)-5- fluoropyrazin-2-yl)-3,4- dihydroisoquinolin-1(2H)-one | 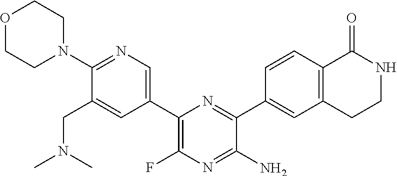 [SMILES: CN(C)Cc1cc(-c2nc(-c3ccc4c(c3)CCNC4=O)c(N)nc2F)cnc1N1CCOCC1] |
| I-238 | 6-(3-amino-6-(3- ((ethyl(methyl)amino)methyl)- 4-(tetrahydro-2H-pyran-4- yl)phenyl)-5-fluoropyrazin-2-yl)- 4-fluoroisoquinolin-1(2H)-one | 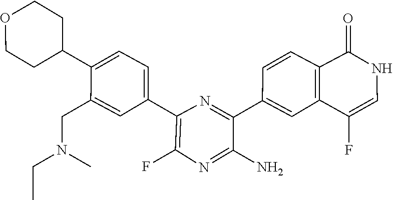 [SMILES: CCN(C)Cc1cc(-c2nc(-c3ccc4c(=O)[nH]cc(F)c4c3)c(N)nc2F)ccc1C1CCOCC1] |
| I-239 | 6-(3-amino-6-(3- ((ethyl(methyl)amino)methyl)- 4-(tetrahydro-2H-pyran-4- yl)phenyl)-5-fluoropyrazin-2-yl)- 4-methylisoquinolin-1(2H)-one | 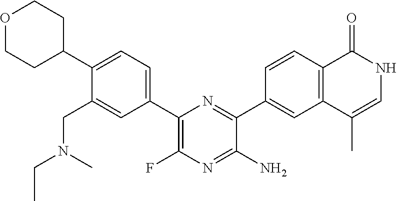 [SMILES: CCN(C)Cc1cc(-c2nc(-c3ccc4c(=O)[nH]cc(C)c4c3)c(N)nc2F)ccc1C1CCOCC1] |
| I-240 | 6-(3-amino-6-(3- ((dimethylamino)methyl)-2- fluoro-4-morpholinophenyl)-5- fluoropyrazin-2-yl)-3,4- dihydroisoquinolin-1(2H)-one | 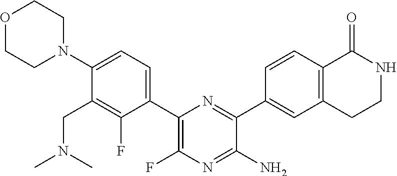 [SMILES: CN(C)Cc1c(N2CCOCC2)ccc(-c2nc(-c3ccc4c(c3)CCNC4=O)c(N)nc2F)c1F] |
| I-241 | 6-(3-amino-6-(3- ((dimethylamino)methyl)-2- fluoro-4-morpholinophenyl)-5- fluoropyrazin-2-yl)-7-fluoro-3,4- dihydroisoquinolin-1(2H)-one | 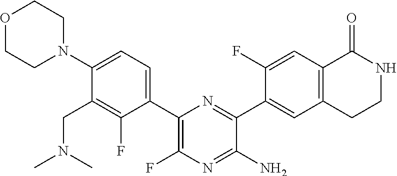 [SMILES: CN(C)Cc1c(N2CCOCC2)ccc(-c2nc(-c3cc4c(cc3F)C(=O)NCC4)c(N)nc2F)c1F] |
| I-242 | 6-(3-amino-6-(3- ((dimethylamino)methyl)-2- fluoro-4-morpholinophenyl)-5- fluoropyrazin-2-yl)-8-fluoro-3,4- dihydroisoquinolin-1(2H)-one | 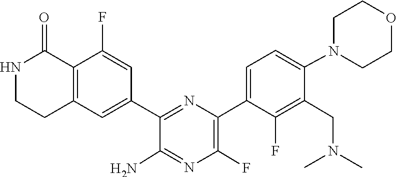 [SMILES: CN(C)Cc1c(N2CCOCC2)ccc(-c2nc(-c3cc(F)c4c(c3)CCNC4=O)c(N)nc2F)c1F] |
| I-243 | 6-(3-amino-6-(3-(azetidin-1- ylmethyl)-2-fluoro-4- morpholinophenyl)-5- fluoropyrazin-2-yl)-3,4- dihydroisoquinolin-1(2H)-one | 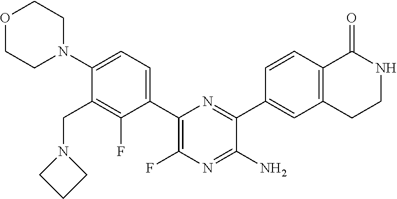 [SMILES: Nc1nc(F)c(-c2ccc(N3CCOCC3)c(CN3CCC3)c2F)nc1-c1ccc2c(c1)CCNC2=O] |
| I-244 | 6-(3-amino-6-(3-(azetidin-1- ylmethyl)-2-fluoro-4- morpholinophenyl)-5- fluoropyrazin-2-yl)-7-fluoro-3,4- dihydroisoquinolin-1(2H)-one | 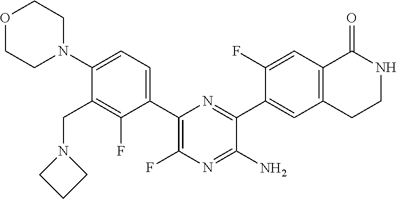 [SMILES: Nc1nc(F)c(-c2ccc(N3CCOCC3)c(CN3CCC3)c2F)nc1-c1cc2c(cc1F)C(=O)NCC2] |
| I-245 | 6-(3-amino-6-(3-(azetidin-1- ylmethyl)-2-fluoro-4- morpholinophenyl)-5- fluoropyrazin-2-yl)-8-fluoro-3,4- dihydroisoquinolin-1(2H)-one | 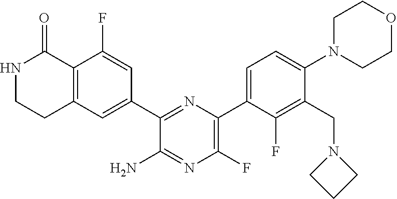 [SMILES: Nc1nc(F)c(-c2ccc(N3CCOCC3)c(CN3CCC3)c2F)nc1-c1cc(F)c2c(c1)CCNC2=O] |
| I-246 | 6-(3-amino-6-(3- ((ethyl(methyl)amino)methyl)- 2-fluoro-4-morpholinophenyl)- 5-fluoropyrazin-2-yl)-7-fluoro- 3,4-dihydroisoquinolin-1(2H)- one | 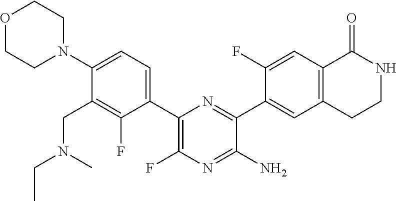 [SMILES: CCN(C)Cc1c(N2CCOCC2)ccc(-c2nc(-c3cc4c(cc3F)C(=O)NCC4)c(N)nc2F)c1F] |
| I-247 | 6-(3-amino-6-(3- ((ethyl(methyl)amino)methyl)- 2-fluoro-4-morpholinophenyl)- 5-fluoropyrazin-2-yl)-8-fluoro- 3,4-dihydroisoquinolin-1(2H)- one | 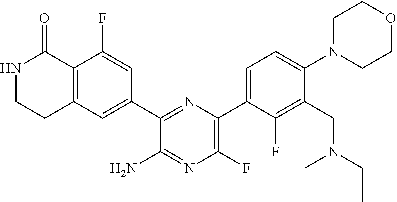 [SMILES: CCN(C)Cc1c(N2CCOCC2)ccc(-c2nc(-c3cc(F)c4c(c3)CCNC4=O)c(N)nc2F)c1F] |
| I-248 | 6-(3-amino-6-(3- ((ethyl(methyl)amino)methyl)- 2-fluoro-4-morpholinophenyl)- 5-fluoropyrazin-2-yl)-3,4- dihydroisoquinolin-1(2H)-one | 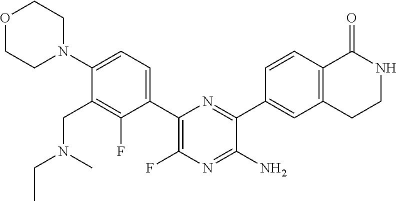 [SMILES: CCN(C)Cc1c(N2CCOCC2)ccc(-c2nc(-c3ccc4c(c3)CCNC4=O)c(N)nc2F)c1F] |
| I-249 | 6-(3-amino-6-(3- ((dimethylamino)methyl)-4- methoxyphenyl)-5- fluoropyrazin-2-yl)-7-fluoro-3,4- dihydroisoquinolin-1(2H)-one | 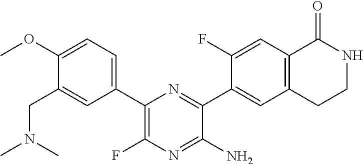 [SMILES: COc1ccc(-c2nc(-c3cc4c(cc3F)C(=O)NCC4)c(N)nc2F)cc1CN(C)C] |
| I-250 | 6-(3-amino-6-(3- ((dimethylamino)methyl)-4- methoxyphenyl)-5- fluoropyrazin-2-yl)-3,4- dihydroisoquinolin-1(2H)-one | 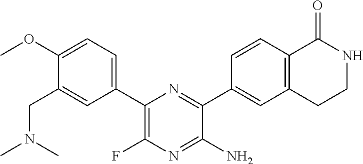 [SMILES: COc1ccc(-c2nc(-c3ccc4c(c3)CCNC4=O)c(N)nc2F)cc1CN(C)C] |
| I-251 | (S)-6-(3-amino-6-(3- (dimethylamino)-2,3- dihydrobenzofuran-5-yl)-5- fluoropyrazin-2-yl)-4- fluoroisoquinolin-1(2H)-one | 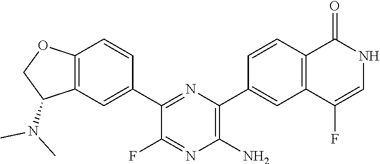 [SMILES: CN(C)[C@@H]1COc2ccc(-c3nc(-c4ccc5c(=O)[nH]cc(F)c5c4)c(N)nc3F)cc21] |
| I-252 | (S)-6-(3-amino-6-(3- (dimethylamino)-2,3- dihydrobenzofuran-5-yl)-5- fluoropyrazin-2-yl)-4- chloroisoquinolin-1(2H)-one | 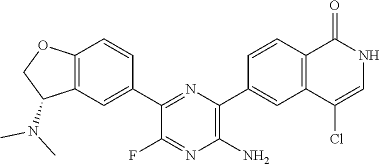 [SMILES: CN(C)[C@@H]1COc2ccc(-c3nc(-c4ccc5c(=O)[nH]cc(Cl)c5c4)c(N)nc3F)cc21] |
| I-253 | (S)-6-(3-amino-6-(3- (dimethylamino)-2,3- dihydrobenzofuran-5-yl)-5- fluoropyrazin-2-yl)-4,8- difluoroisoquinolin-1(2H)-one | 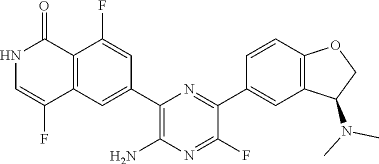 [SMILES: CN(C)[C@@H]1COc2ccc(-c3nc(-c4cc(F)c5c(=O)[nH]cc(F)c5c4)c(N)nc3F)cc21] |
| I-254 | (S)-6-(3-amino-6-(3- (dimethylamino)-2,3- dihydrobenzofuran-5-yl)-5- fluoropyrazin-2-yl)-8-fluoro-3,4- dihydroisoquinolin-1(2H)-one | 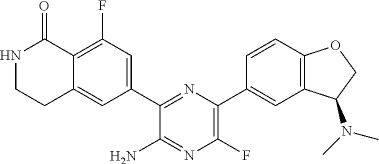 [SMILES: CN(C)[C@@H]1COc2ccc(-c3nc(-c4cc(F)c5c(c4)CCNC5=O)c(N)nc3F)cc21] |
| I-255 | (S)-6-(3-amino-6-(3- (dimethylamino)-2,3- dihydrobenzofuran-5-yl)-5- fluoropyrazin-2-yl)-7-fluoro-3,4- dihydroisoquinolin-1(2H)-one | 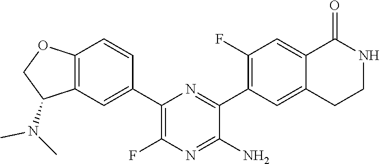 [SMILES: CN(C)[C@@H]1COc2ccc(-c3nc(-c4cc5c(cc4F)C(=O)NCC5)c(N)nc3F)cc21] |
| I-256 | (S)-6-(3-amino-6-(3- (dimethylamino)-2,3- dihydrobenzofuran-5-yl)-5- fluoropyrazin-2-yl)-4,4-difluoro- 3,4-dihydroisoquinolin-1(2H)- one | 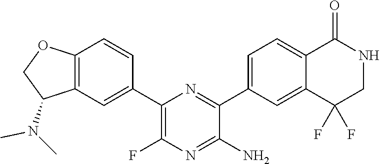 [SMILES: CN(C)[C@@H]1COc2ccc(-c3nc(-c4ccc5c(c4)C(F)(F)CNC5=O)c(N)nc3F)cc21] |
| I-257 | 6-(3-amino-6-(3- ((dimethylamino)methyl)-4- ((2R,6S)-2,6- dimethylmorpholino)phenyl)-5- fluoropyrazin-2-yl)-4,4-difluoro- 3,4-dihydroisoquinolin-1(2H)- one | 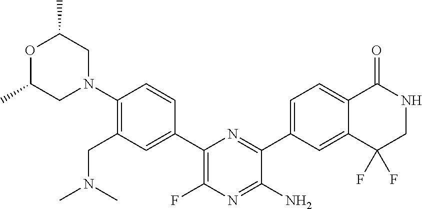 [SMILES: C[C@@H]1CN(c2ccc(-c3nc(-c4ccc5c(c4)C(F)(F)CNC5=O)c(N)nc3F)cc2CN(C)C)C[C@H](C)O1] |
| I-258 | 6-(3-amino-6-(5- ((dimethylamino)methyl)-2- fluoro-4-morpholinophenyl)-5- fluoropyrazin-2-yl)-3,4- dihydroisoquinolin-1(2H)-one | 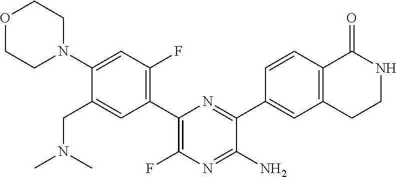 [SMILES: CN(C)Cc1cc(-c2nc(-c3ccc4c(c3)CCNC4=O)c(N)nc2F)c(F)cc1N1CCOCC1] |
| I-259 | 6-(3-amino-6-(5- ((dimethylamino)methyl)-2- fluoro-4-morpholinophenyl)-5- fluoropyrazin-2-yl)-4- fluoroisoquinolin-1(2H)-one | 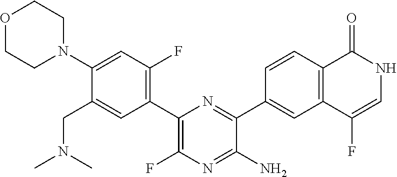 [SMILES: CN(C)Cc1cc(-c2nc(-c3ccc4c(=O)[nH]cc(F)c4c3)c(N)nc2F)c(F)cc1N1CCOCC1] |
| I-260 | 7′-(3-amino-5-fluoro-6-(4-(4- isopropylpiperazin-1- yl)phenyl)pyrazin-2-yl)-2′,3′- dihydro-4′H- spiro[cyclopropane-1,1′- isoquinolin]-4′-one | 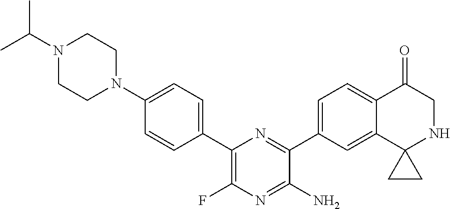 [SMILES: CC(C)N1CCN(c2ccc(-c3nc(-c4ccc5c(c4)C4(CC4)NCC5=O)c(N)nc3F)cc2)CC1] |
| I-261 | 6-(3-amino-6-(5- ((dimethylamino)methyl)-2- fluoro-4-morpholinophenyl)-5- fluoropyrazin-2-yl)-4- methylisoquinolin-1(2H)-one | 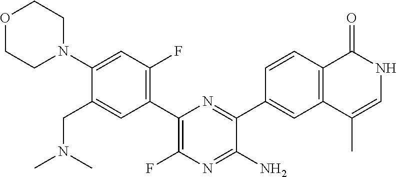 [SMILES: Cc1c[nH]c(=O)c2ccc(-c3nc(-c4cc(CN(C)C)c(N5CCOCC5)cc4F)c(F)nc3N)cc12] |
| I-262 | 6-(3-amino-6-(5- ((dimethylamino)methyl)-2,3- difluoro-4-morpholinophenyl)- 5-fluoropyrazin-2-yl)-3,4- dihydroisoquinolin-1(2H)-one | 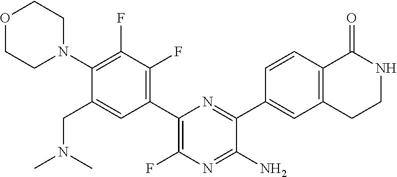 [SMILES: CN(C)Cc1cc(-c2nc(-c3ccc4c(c3)CCNC4=O)c(N)nc2F)c(F)c(F)c1N1CCOCC1] |
| I-263 | 6-(3-amino-6-(5- ((dimethylamino)methyl)-2,3- difluoro-4-morpholinophenyl)- 5-fluoropyrazin-2-yl)-4- fluoroisoquinolin-1(2H)-one | 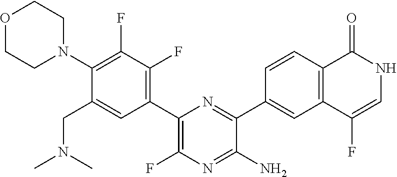 [SMILES: CN(C)Cc1cc(-c2nc(-c3ccc4c(=O)[nH]cc(F)c4c3)c(N)nc2F)c(F)c(F)c1N1CCOCC1] |
| I-264 | 6-(3-amino-6-(5- ((dimethylamino)methyl)-2,3- difluoro-4-morpholinophenyl)- 5-fluoropyrazin-2-yl)-4- methylisoquinolin-1(2H)-one | 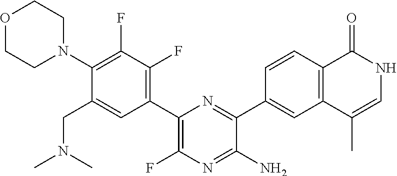 [SMILES: Cc1c[nH]c(=O)c2ccc(-c3nc(-c4cc(CN(C)C)c(N5CCOCC5)c(F)c4F)c(F)nc3N)cc12] |
| I-265 | 6-(3-amino-6-(3- ((dimethylamino)methyl)-5- fluoro-4-morpholinophenyl)-5- fluoropyrazin-2-yl)-4- fluoroisoquinolin-1(2H)-one | 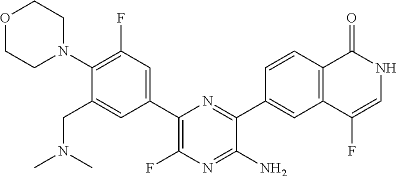 [SMILES: CN(C)Cc1cc(-c2nc(-c3ccc4c(=O)[nH]cc(F)c4c3)c(N)nc2F)cc(F)c1N1CCOCC1] |
| I-266 | 6-(3-amino-6-(3- ((dimethylamino)methyl)-5- fluoro-4-morpholinophenyl)-5- fluoropyrazin-2-yl)-4- methylisoquinolin-1(2H)-one | 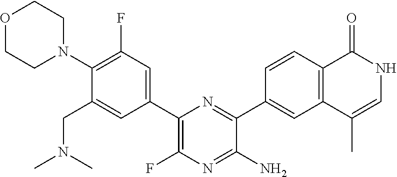 [SMILES: Cc1c[nH]c(=O)c2ccc(-c3nc(-c4cc(F)c(N5CCOCC5)c(CN(C)C)c4)c(F)nc3N)cc12] |
| I-267 | 6-(3-amino-6-(3- ((dimethylamino)methyl)-4- methoxyphenyl)-5- fluoropyrazin-2-yl)-4- fluoroisoquinolin-1(2H)-one | 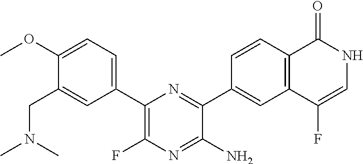 [SMILES: COc1ccc(-c2nc(-c3ccc4c(=O)[nH]cc(F)c4c3)c(N)nc2F)cc1CN(C)C] |
| I-268 | 6-(3-amino-6-(8- ((dimethylamino)methyl)chroman- 6-yl)-5-fluoropyrazin-2-yl)-4- fluoroisoquinolin-1(2H)-one | 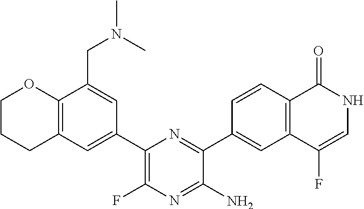 [SMILES: CN(C)Cc1cc(-c2nc(-c3ccc4c(=O)[nH]cc(F)c4c3)c(N)nc2F)cc2c1OCCC2] |
| I-269 | 6-(3-amino-6-(8- ((dimethylamino)methyl)-2,3- dihydrobenzo[b][1,4]dioxin-6- yl)-5-fluoropyrazin-2-yl)-4- fluoroisoquinolin-1(2H)-one | 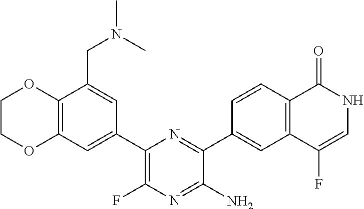 [SMILES: CN(C)Cc1cc(-c2nc(-c3ccc4c(=O)[nH]cc(F)c4c3)c(N)nc2F)cc2c1OCCO2] |
| I-270 | 6-(3-amino-6-(4- (difluoromethoxy)-3- ((dimethylamino)methyl)phenyl)- 5-fluoropyrazin-2-yl)-4- fluoroisoquinolin-1(2H)-one | 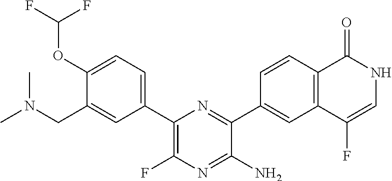 [SMILES: CN(C)Cc1cc(-c2nc(-c3ccc4c(=O)[nH]cc(F)c4c3)c(N)nc2F)ccc1OC(F)F] |
| I-271 | 6-(3-amino-6-(3-(2- aminopropan-2-yl)-4- fluorophenyl)-5-fluoropyrazin- 2-yl)-3,4-dihydroisoquinolin- 1(2H)-one | 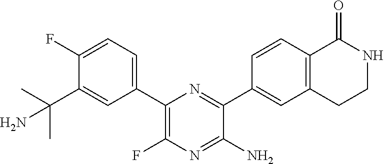 [SMILES: CC(C)(N)c1cc(-c2nc(-c3ccc4c(c3)CCNC4=O)c(N)nc2F)ccc1F] |
| I-272 | 6-(3-amino-6-(3- ((dimethylamino)methyl)-4- fluorophenyl)-5-fluoropyrazin- 2-yl)-3,4-dihydroisoquinolin- 1(2H)-one | 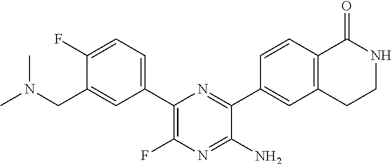 [SMILES: CN(C)Cc1cc(-c2nc(-c3ccc4c(c3)CCNC4=O)c(N)nc2F)ccc1F] |
| I-273 | 6-(3-amino-6-(3- ((dimethylamino)methyl)-4- fluorophenyl)-5-fluoropyrazin- 2-yl)-7-fluoro-3,4- dihydroisoquinolin-1(2H)-one | 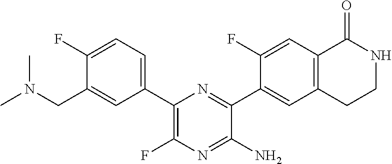 [SMILES: CN(C)Cc1cc(-c2nc(-c3cc4c(cc3F)C(=O)NCC4)c(N)nc2F)ccc1F] |
| I-274 | 6-(3-amino-6-(3- ((dimethylamino)methyl)-4- fluorophenyl)-5-fluoropyrazin- 2-yl)-8-fluoro-3,4- dihydroisoquinolin-1(2H)-one | 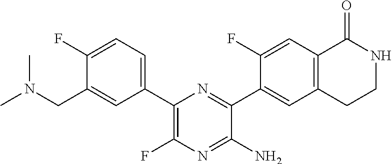 [SMILES: CN(C)Cc1cc(-c2nc(-c3cc4c(cc3F)C(=O)NCC4)c(N)nc2F)ccc1F] |
| I-275 | 6-(3-amino-6-(3- ((dimethylamino)methyl)-4- fluorophenyl)-5-fluoropyrazin- 2-yl)-4-methylisoquinolin- 1(2H)-one | 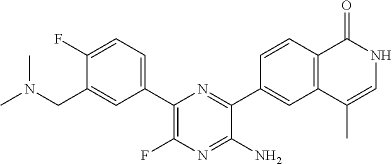 [SMILES: Cc1c[nH]c(=O)c2ccc(-c3nc(-c4ccc(F)c(CN(C)C)c4)c(F)nc3N)cc12] |
| I-276 | 6-(3-amino-6-(3- ((dimethylamino)methyl)-4- fluorophenyl)-5-fluoropyrazin- 2-yl)-4-fluoroisoquinolin-1(2H)- one | 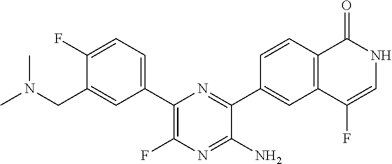 [SMILES: CN(C)Cc1cc(-c2nc(-c3ccc4c(=O)[nH]cc(F)c4c3)c(N)nc2F)ccc1F] |
| I-277 | 6-(3-amino-6-(3- ((dimethylamino)methyl)phenyl)- 5-fluoropyrazin-2-yl)-3,4- dihydroisoquinolin-1(2H)-one | 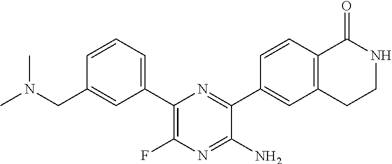 [SMILES: CN(C)Cc1cccc(-c2nc(-c3ccc4c(c3)CCNC4=O)c(N)nc2F)c1] |
| I-278 | 6-(3-amino-6-(3- ((dimethylamino)methyl)phenyl)- 5-fluoropyrazin-2-yl)-7-fluoro- 3,4-dihydroisoquinolin-1(2H)- one | 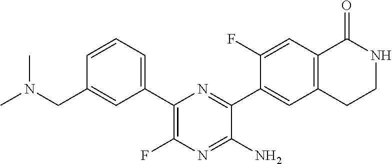 [SMILES: CN(C)Cc1cccc(-c2nc(-c3cc4c(cc3F)C(=O)NCC4)c(N)nc2F)c1] |
| I-279 | 6-(3-amino-6-(3- ((dimethylamino)methyl)phenyl)- 5-fluoropyrazin-2-yl)-8-fluoro- 3,4-dihydroisoquinolin-1(2H)- one | 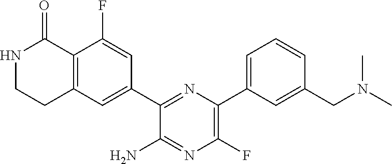 [SMILES: CN(C)Cc1cccc(-c2nc(-c3cc(F)c4c(c3)CCNC4=O)c(N)nc2F)c1] |
| I-280 | 6-(3-amino-6-(3- ((dimethylamino)methyl)phenyl)- 5-fluoropyrazin-2-yl)-4- methylisoquinolin-1(2H)-one | 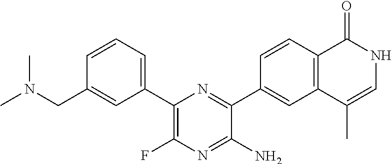 [SMILES: Cc1c[nH]c(=O)c2ccc(-c3nc(-c4cccc(CN(C)C)c4)c(F)nc3N)cc12] |
| I-281 | 6-(3-amino-6-(3- ((dimethylamino)methyl)phenyl)- 5-fluoropyrazin-2-yl)-4- fluoroisoquinolin-1(2H)-one | 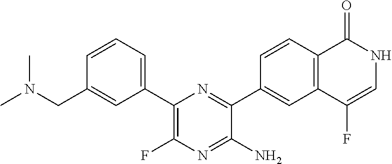 [SMILES: CN(C)Cc1cccc(-c2nc(-c3ccc4c(=O)[nH]cc(F)c4c3)c(N)nc2F)c1] |
| I-282 | 6-(3-amino-6-(4-(3,6-dihydro- 2H-pyran-4-yl)-3- ((ethyl(methyl)amino)methyl) phenyl)-5-fluoropyrazin-2-yl)-4- fluoroisoquinolin-1(2H)-one | 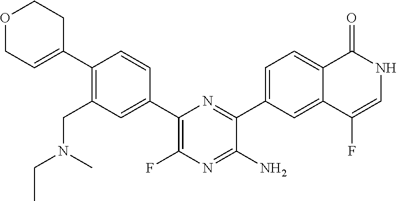 [SMILES: CCN(C)Cc1cc(-c2nc(-c3ccc4c(=O)[nH]cc(F)c4c3)c(N)nc2F)ccc1C1=CCOCC1] |
| I-283 | (S)-6-(3-amino-6-(3- (dimethylamino)-2,3- dihydrobenzofuran-5-yl)-5- fluoropyrazin-2-yl)isoquinolin- 1(2H)-one | 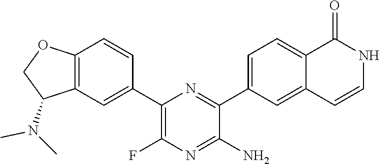 [SMILES: CN(C)[C@@H]1COc2ccc(-c3nc(-c4ccc5c(=O)[nH]ccc5c4)c(N)nc3F)cc21] |
| I-284 | (S)-6-(3-amino-6-(3- (dimethylamino)-2,3- dihydrobenzofuran-5-yl)-5- fluoropyrazin-2-yl)-4- methylisoquinolin-1(2H)-one | 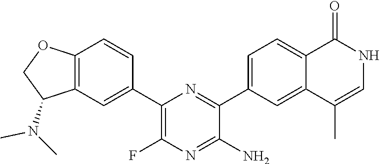 [SMILES: Cc1c[nH]c(=O)c2ccc(-c3nc(-c4ccc5c(c4)[C@H](N(C)C)CO5)c(F)nc3N)cc12] |
| I-285 | (R)-6-(3-amino-6-(4- (dimethylamino)chroman-6-yl)- 5-fluoropyrazin-2-yl)-4- methylisoquinolin-1(2H)-one | 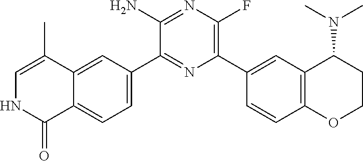 [SMILES: Cc1c[nH]c(=O)c2ccc(-c3nc(-c4ccc5c(c4)[C@H](N(C)C)CCO5)c(F)nc3N)cc12] |
| I-286 | (R)-6-(3-amino-6-(4- (dimethylamino)chroman-6-yl)- 5-fluoropyrazin-2- yl)isoquinolin-1(2H)-one | 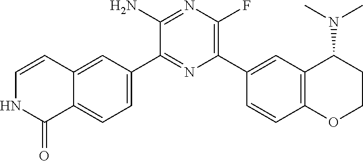 [SMILES: CN(C)[C@@H]1CCOc2ccc(-c3nc(-c4ccc5c(=O)[nH]ccc5c4)c(N)nc3F)cc21] |
| I-287 | (R)-6-(3-amino-6-(4- (dimethylamino)chroman-6-yl)- 5-fluoropyrazin-2-yl)-3,4- dihydroisoquinolin-1(2H)-one | 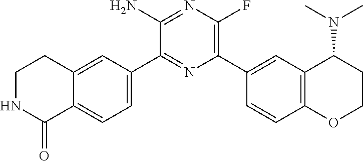 [SMILES: CN(C)[C@@H]1CCOc2ccc(-c3nc(-c4ccc5c(c4)CCNC5=O)c(N)nc3F)cc21] |
| I-288 | (R)-6-(3-amino-6-(4- (dimethylamino)chroman-6-yl)- 5-fluoropyrazin-2-yl)-7-fluoro- 3,4-dihydroisoquinolin-1(2H)- one | 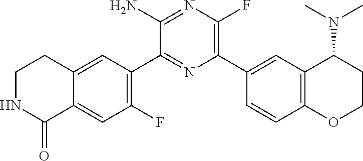 [SMILES: CN(C)[C@@H]1CCOc2ccc(-c3nc(-c4cc5c(cc4F)C(=O)NCC5)c(N)nc3F)cc21] |
| I-289 | (R)-6-(3-amino-6-(4- (dimethylamino)chroman-6-yl)- 5-fluoropyrazin-2-yl)-8-fluoro- 3,4-dihydroisoquinolin-1(2H)- one | 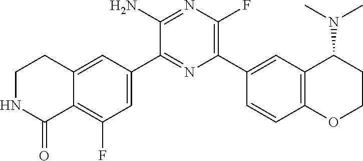 [SMILES: CN(C)[C@@H]1CCOc2ccc(-c3nc(-c4cc(F)c5c(c4)CCNC5=O)c(N)nc3F)cc21] |
| I-290 | 6-(3-amino-6-(3-(1- (dimethylamino)ethyl)-4- methoxyphenyl)-5- fluoropyrazin-2-yl)-3,4- dihydroisoquinolin-1(2H)-one | 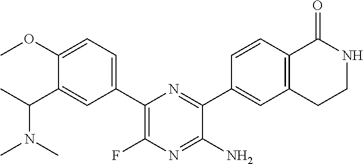 [SMILES: COc1ccc(-c2nc(-c3ccc4c(c3)CCNC4=O)c(N)nc2F)cc1C(C)N(C)C] |
| I-291 | 6-(3-amino-6-(3-(1- (dimethylamino)ethyl)-4- methoxyphenyl)-5- fluoropyrazin-2-yl)-4- fluoroisoquinolin-1(2H)-one | 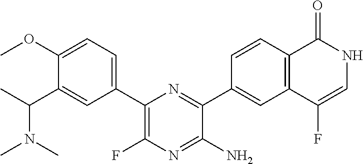 [SMILES: COc1ccc(-c2nc(-c3ccc4c(=O)[nH]cc(F)c4c3)c(N)nc2F)cc1C(C)N(C)C] |
| I-292 | 5-(5-amino-3-fluoro-6-(1-oxo- 1,2,3,4-tetrahydroisoquinolin-6- yl)pyrazin-2-yl)-2- methoxybenzonitrile | 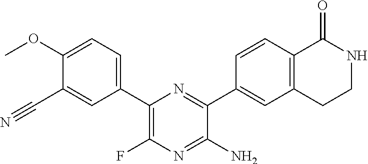 [SMILES: COc1ccc(-c2nc(-c3ccc4c(c3)CCNC4=O)c(N)nc2F)cc1C#N] |
| I-293 | 6-(3-amino-6-(3- ((dimethylamino)methyl)-4- (isopropylsulfonyl)phenyl)-5- fluoropyrazin-2-yl)-4- fluoroisoquinolin-1(2H)-one | 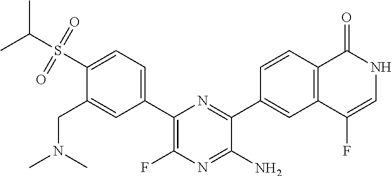 [SMILES: CC(C)S(=O)(=O)c1ccc(-c2nc(-c3ccc4c(=O)[nH]cc(F)c4c3)c(N)nc2F)cc1CN(C)C] |
| I-294 | 6-(3-amino-6-(2,5-dimethyl- 1,2,3,4-tetrahydroisoquinolin-7- yl)-5-fluoropyrazin-2-yl)-3,4- dihydroisoquinolin-1(2H)-one | 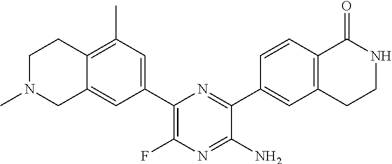 [SMILES: Cc1cc(-c2nc(-c3ccc4c(c3)CCNC4=O)c(N)nc2F)cc2c1CCN(C)C2] |
| I-295 | 6-(3-amino-6-(2,5-dimethyl- 1,2,3,4-tetrahydroisoquinolin-7- yl)-5-fluoropyrazin-2-yl)-4- methylisoquinolin-1(2H)-one | 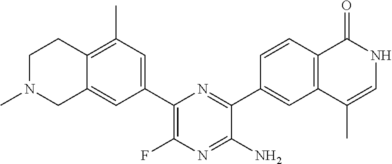 [SMILES: Cc1cc(-c2nc(-c3ccc4c(=O)[nH]cc(C)c4c3)c(N)nc2F)cc2c1CCN(C)C2] |
| I-296 | 6-(3-amino-6-(2,5-dimethyl- 1,2,3,4-tetrahydroisoquinolin-7- yl)-5-fluoropyrazin-2-yl)-4- fluoroisoquinolin-1(2H)-one | 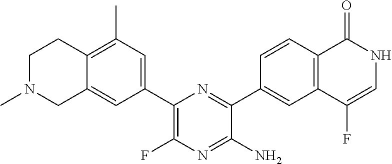 [SMILES: Cc1cc(-c2nc(-c3ccc4c(=O)[nH]cc(F)c4c3)c(N)nc2F)cc2c1CCN(C)C2] |
| I-297 | 6-(3-amino-6-(3- ((ethyl(methyl)amino)methyl)- 4-methoxy-5-methylphenyl)-5- fluoropyrazin-2-yl)-3,4- dihydroisoquinolin-1(2H)-one | 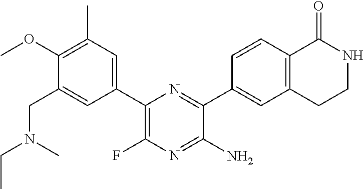 [SMILES: CCN(C)Cc1cc(-c2nc(-c3ccc4c(c3)CCNC4=O)c(N)nc2F)cc(C)c1OC] |
| I-298 | 6-(3-amino-6-(3- ((ethyl(methyl)amino)methyl)- 4-methoxy-5-methylphenyl)-5- fluoropyrazin-2-yl)-4- fluoroisoquinolin-1(2H)-one | 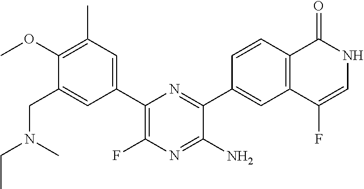 [SMILES: CCN(C)Cc1cc(-c2nc(-c3ccc4c(=O)[nH]cc(F)c4c3)c(N)nc2F)cc(C)c1OC] |
| I-299 | 6-(3-amino-6-(3- ((ethyl(methyl)amino)methyl)- 4-methoxy-5-methylphenyl)-5- fluoropyrazin-2-yl)-4- methylisoquinolin-1(2H)-one | 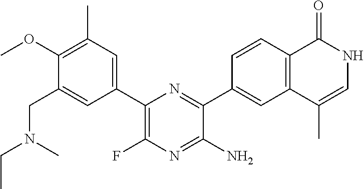 [SMILES: CCN(C)Cc1cc(-c2nc(-c3ccc4c(=O)[nH]cc(C)c4c3)c(N)nc2F)cc(C)c1OC] |
| I-300 | 6-(3-amino-6-(3- ((ethyl(methyl)amino)methyl)- 4-methoxyphenyl)-5- fluoropyrazin-2-yl)-4- fluoroisoquinolin-1(2H)-one | 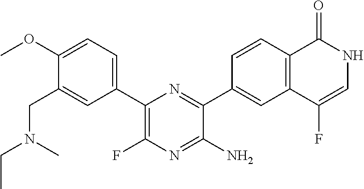 [SMILES: CCN(C)Cc1cc(-c2nc(-c3ccc4c(=O)[nH]cc(F)c4c3)c(N)nc2F)ccc1OC] |
| I-301 | 6-(3-amino-6-(3- ((ethyl(methyl)amino)methyl)- 4-methoxyphenyl)-5- fluoropyrazin-2-yl)-4- methylisoquinolin-1(2H)-one | 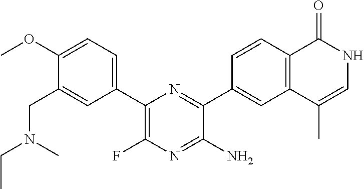 [SMILES: CCN(C)Cc1cc(-c2nc(-c3ccc4c(=O)[nH]cc(C)c4c3)c(N)nc2F)ccc1OC] |
| I-302 | 6-(3-amino-6-(3-(1- (dimethylamino)ethyl)-4- hydroxyphenyl)-5- fluoropyrazin-2-yl)-4- fluoroisoquinolin-1(2H)-one | 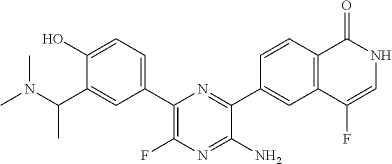 [SMILES: CC(c1cc(-c2nc(-c3ccc4c(=O)[nH]cc(F)c4c3)c(N)nc2F)ccc1O)N(C)C] |
| I-303 | 6-(3-amino-6-(3- ((ethyl(methyl)amino)methyl)- 4-methoxy-5-methylphenyl)-5- fluoropyrazin-2-yl)-7-fluoro-3,4- dihydroisoquinolin-1(2H)-one | 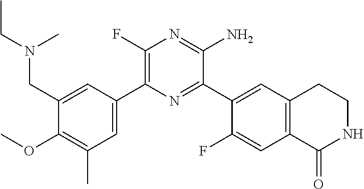 [SMILES: CCN(C)Cc1cc(-c2nc(-c3cc4c(cc3F)C(=O)NCC4)c(N)nc2F)cc(C)c1OC] |
| I-304 | 6-(3-amino-6-(3-(1- (dimethylamino)ethyl)-4- hydroxyphenyl)-5- fluoropyrazin-2-yl)-4- methylisoquinolin-1(2H)-one | 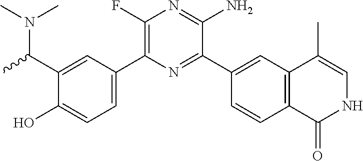 [SMILES: Cc1c[nH]c(=O)c2ccc(-c3nc(-c4ccc(O)c(C(C)N(C)C)c4)c(F)nc3N)cc12] |
| I-305 | 6-(3-amino-6-(3- ((dimethylamino)methyl)-4- hydroxyphenyl)-5- fluoropyrazin-2-yl)-4- fluoroisoquinolin-1(2H)-one | 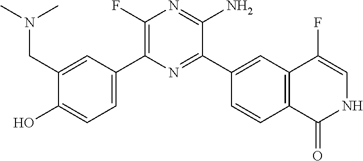 [SMILES: CN(C)Cc1cc(-c2nc(-c3ccc4c(=O)[nH]cc(F)c4c3)c(N)nc2F)ccc1O] |
| I-306 | 6-(3-amino-6-(3- ((dimethylamino)methyl)-4- hydroxyphenyl)-5- fluoropyrazin-2-yl)-4- methylisoquinolin-1(2H)-one | 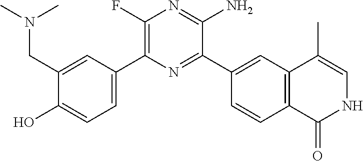 [SMILES: Cc1c[nH]c(=O)c2ccc(-c3nc(-c4ccc(O)c(CN(C)C)c4)c(F)nc3N)cc12] |

or a pharmaceutically acceptable salt, solvate and/or prodrug thereof.

In embodiments of the present application, the compounds described herein may have at least one asymmetric center. Where compounds possess more than one asymmetric center, they may exist as diastereomers. It is to be understood that all such isomers and mixtures thereof in any proportion are encompassed within the scope of the present application. It is to be further understood that while the stereochemistry of the compounds may be as shown in any given compound listed herein, such compounds may also contain certain amounts (for example, less than 20%, suitably less than 10%, more suitably less than 5%) of compounds of the present application having an alternate stereochemistry. It is intended that any optical isomers, as separated, pure or partially purified optical isomers or racemic mixtures thereof are included within the scope of the present application.

The compounds of the present application may also exist in different tautomeric forms and it is intended that any tautomeric forms which the compounds form, as well as mixtures thereof, are included within the scope of the present application.

The compounds of the present application may further exist in varying polymorphic forms and it is contemplated that any polymorphs, or mixtures thereof, which form are included within the scope of the present application.

In an embodiment the pharmaceutically acceptable salt is an acid addition salt or a base addition salt. The selection of a suitable salt may be made by a person skilled in the art (see, for example, S. M. Berge, et al., “Pharmaceutical Salts,” J. Pharm. Sci. 1977, 66, 1-19).

An acid addition salt suitable for, or compatible with, the treatment of subjects is any non-toxic organic or inorganic acid addition salt of any basic compound. Basic compounds that form an acid addition salt include, for example, compounds comprising an amine group. Illustrative inorganic acids which form suitable salts include hydrochloric, hydrobromic, sulfuric, nitric and phosphoric acids, as well as acidic metal salts such as sodium monohydrogen orthophosphate and potassium hydrogen sulfate. Illustrative organic acids which form suitable salts include mono-, di- and tricarboxylic acids. Illustrative of such organic acids are, for example, acetic, trifluoroacetic, propionic, glycolic, lactic, pyruvic, malonic, succinic, glutaric, fumaric, malic, tartaric, citric, ascorbic, maleic, hydroxymaleic, benzoic, hydroxybenzoic, phenylacetic, cinnamic, mandelic, salicylic, 2-phenoxybenzoic, p-toluenesulfonic acid and other sulfonic acids such as methanesulfonic acid, ethanesulfonic acid and 2-hydroxyethanesulfonic acid. In an embodiment, the mono- or di-acid salts are formed, and such salts exist in either a hydrated, solvated or substantially anhydrous form. In general, acid addition salts are more soluble in water and various hydrophilic organic solvents, and generally demonstrate higher melting points in comparison to their free base forms. The selection criteria for the appropriate salt will be known to one skilled in the art. Other non-pharmaceutically acceptable salts such as but not limited to oxalates may be used, for example in the isolation of compounds of the application for laboratory use, or for subsequent conversion to a pharmaceutically acceptable acid addition salt.

A base addition salt suitable for, or compatible with, the treatment of subjects is any non-toxic organic or inorganic base addition salt of any acidic compound. Acidic compounds that form a basic addition salt include, for example, compounds comprising a carboxylic acid group. Illustrative inorganic bases which form suitable salts include lithium, sodium, potassium, calcium, magnesium or barium hydroxide as well as ammonia. Illustrative organic bases which form suitable salts include aliphatic, alicyclic or aromatic organic amines such as isopropylamine, methylamine, trimethylamine, picoline, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins, and the like. Exemplary organic bases are isopropylamine, diethylamine, ethanolamine, trimethylamine, dicyclohexylamine, choline, and caffeine. The selection of the appropriate salt may be useful, for example, so that an ester functionality, if any, elsewhere in a compound is not hydrolyzed. The selection criteria for the appropriate salt will be known to one skilled in the art.

Solvates of compounds of the application include, for example, those made with solvents that are pharmaceutically acceptable. Examples of such solvents include water (resulting solvate is called a hydrate) and ethanol and the like.

Prodrugs of the compounds of the present application may be, for example, conventional esters formed with available hydroxy, thiol, amino or carboxyl groups. Some common esters which have been utilized as prodrugs are phenyl esters, aliphatic (C₁-C₂₄) esters, acyloxymethyl esters, carbamates and amino acid esters.

The compounds of the present application also include compounds having alternate isotopes, including radioactive and non-radioactive isotopes, for any of the atoms. For example, in an embodiment, the compounds of the application include compounds wherein one or more available hydrogen atoms have been substituted with deuterium. in an embodiment, the compounds of the application include compounds wherein one or more available carbon atoms have been substituted with ¹³C.

The compounds of the present application are suitably formulated in a conventional manner into compositions using one or more carriers. Accordingly, the present application also includes a composition comprising one or more compounds of the application and a carrier. The compounds of the application are suitably formulated into pharmaceutical compositions for administration to subjects in a biologically compatible form suitable for administration in vivo. Accordingly, the present application further includes a pharmaceutical composition comprising one or more compounds of the application and a pharmaceutically acceptable carrier.

A compound of the application including salts and/or solvates thereof is suitably used on their own but will generally be administered in the form of a composition in which the one or more compounds of the application (the active ingredient) is in association with an acceptable carrier. Depending on the mode of administration, the composition will comprise from about 0.05 wt % to about 99 wt % or about 0.10 wt % to about 70 wt %, of the active ingredient, and from about 1 wt % to about 99.95 wt % or about 30 wt % to about 99.90 wt % of an acceptable carrier, all percentages by weight being based on the total composition.

The compounds of the application may be administered to a subject in a variety of forms depending on the selected route of administration, as will be understood by those skilled in the art. A compound of the application may be administered, for example, by oral, parenteral, buccal, sublingual, nasal, rectal, patch, pump or transdermal administration and the pharmaceutical compositions formulated accordingly. Administration can be by means of a pump for periodic or continuous delivery. Conventional procedures and ingredients for the selection and preparation of suitable compositions are described, for example, in Remington's Pharmaceutical Sciences (2000-20th edition) and in The United States Pharmacopeia: The National Formulary (USP 24 NF19) published in 1999.

Parenteral administration includes intravenous, intra-arterial, intraperitoneal, subcutaneous, intramuscular, transepithelial, nasal, intrapulmonary (for example, by use of an aerosol), intrathecal, rectal and topical (including the use of a patch or other transdermal delivery device) modes of administration. Parenteral administration may be by continuous infusion over a selected period of time.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists.

A compound of the application may be orally administered, for example, with an inert diluent or with an assimilable edible carrier, or it may be enclosed in hard or soft shell gelatin capsules, or it may be compressed into tablets, or it may be incorporated directly with the food of the diet. For oral therapeutic administration, the compound may be incorporated with excipient and used in the form of ingestible tablets, buccal tablets, troches, capsules, caplets, pellets, granules, lozenges, chewing gum, powders, syrups, elixirs, wafers, aqueous solutions and suspensions, and the like. In the case of tablets, carriers that are used include lactose, corn starch, sodium citrate and salts of phosphoric acid. Pharmaceutically acceptable excipients include binding agents (e.g., pregelatinized maize starch, polyvinylpyrrolidone or hydroxypropyl methylcellulose); fillers (e.g., lactose, microcrystalline cellulose or calcium phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. In the case of tablets, capsules, caplets, pellets or granules for oral administration, pH sensitive enteric coatings, such as Eudragits™ designed to control the release of active ingredients are optionally used. Oral dosage forms also include modified release, for example immediate release and timed-release, formulations. Examples of modified-release formulations include, for example, sustained-release (SR), extended-release (ER, XR, or XL), time-release or timed-release, controlled-release (CR), or continuous-release (CR or Contin), employed, for example, in the form of a coated tablet, an osmotic delivery device, a coated capsule, a microencapsulated microsphere, an agglomerated particle, e.g., as of molecular sieving type particles, or, a fine hollow permeable fiber bundle, or chopped hollow permeable fibers, agglomerated or held in a fibrous packet. Timed-release compositions can be formulated, e.g. liposomes or those wherein the active compound is protected with differentially degradable coatings, such as by microencapsulation, multiple coatings, etc. Liposome delivery systems include, for example, small unilamellar vesicles, large unilamellar vesicles and multilamellar vesicles. Liposomes can be formed from a variety of phospholipids, such as cholesterol, stearylamine or phosphatidylcholines. For oral administration in a capsule form, useful carriers or diluents include lactose and dried corn starch.

Liquid preparations for oral administration may take the form of, for example, solutions, syrups or suspensions, or they are suitably presented as a dry product for constitution with water or other suitable vehicle before use. When aqueous suspensions and/or emulsions are administered orally, the compound of the application is suitably suspended or dissolved in an oily phase that is combined with emulsifying and/or suspending agents. If desired, certain sweetening and/or flavoring and/or coloring agents may be added. Such liquid preparations for oral administration may be prepared by conventional means with pharmaceutically acceptable additives such as suspending agents (e.g., sorbitol syrup, methyl cellulose or hydrogenated edible fats); emulsifying agents (e.g., lecithin or acacia); non-aqueous vehicles (e.g., almond oil, oily esters or ethyl alcohol); and preservatives (e.g., methyl or propyl p-hydroxybenzoates or sorbic acid). Useful diluents include lactose and high molecular weight polyethylene glycols.

It is also possible to freeze-dry the compounds of the application and use the lyophilizates obtained, for example, for the preparation of products for injection.

A compound of the application may also be administered parenterally. Solutions of a compound of the application can be prepared in water suitably mixed with a surfactant such as hydroxypropylcellulose. Dispersions can also be prepared in glycerol, liquid polyethylene glycols, DMSO and mixtures thereof with or without alcohol, and in oils. Under ordinary conditions of storage and use, these preparations contain a preservative to prevent the growth of microorganisms. A person skilled in the art would know how to prepare suitable formulations. For parenteral administration, sterile solutions of the compounds of the application are usually prepared, and the pH of the solutions are suitably adjusted and buffered. For intravenous use, the total concentration of solutes should be controlled to render the preparation isotonic. For ocular administration, ointments or droppable liquids may be delivered by ocular delivery systems known to the art such as applicators or eye droppers. Such compositions can include mucomimetics such as hyaluronic acid, chondroitin sulfate, hydroxypropyl methylcellulose or polyvinyl alcohol, preservatives such as sorbic acid, EDTA or benzyl chromium chloride, and the usual quantities of diluents or carriers. For pulmonary administration, diluents or carriers will be selected to be appropriate to allow the formation of an aerosol.

The compounds of the application may be formulated for parenteral administration by injection, including using conventional catheterization techniques or infusion. Formulations for injection may be presented in unit dosage form, e.g., in ampoules or in multi-dose containers, with an added preservative. The compositions may take such forms as sterile suspensions, solutions or emulsions in oily or aqueous vehicles, and may contain formulating agents such as suspending, stabilizing and/or dispersing agents. In all cases, the form must be sterile and must be fluid to the extent that easy syringability exists. Alternatively, the compounds of the application are suitably in a sterile powder form for reconstitution with a suitable vehicle, e.g., sterile pyrogen-free water, before use.

Compositions for nasal administration may conveniently be formulated as aerosols, drops, gels and powders.

For intranasal administration or administration by inhalation, the compounds of the application are conveniently delivered in the form of a solution, dry powder formulation or suspension from a pump spray container that is squeezed or pumped by the patient or as an aerosol spray presentation from a pressurized container or a nebulizer. Aerosol formulations typically comprise a solution or fine suspension of the active substance in a physiologically acceptable aqueous or non-aqueous solvent and are usually presented in single or multidose quantities in sterile form in a sealed container, which can take the form of a cartridge or refill for use with an atomising device. Alternatively, the sealed container may be a unitary dispensing device such as a single dose nasal inhaler or an aerosol dispenser fitted with a metering valve which is intended for disposal after use. Where the dosage form comprises an aerosol dispenser, it will contain a propellant which can be a compressed gas such as compressed air or an organic propellant such as fluorochlorohydrocarbon. Suitable propellants include but are not limited to dichlorodifluoromethane, trichlorofluoromethane, dichlorotetrafluoroethane, heptafluoroalkanes, carbon dioxide or another suitable gas. In the case of a pressurized aerosol, the dosage unit is suitably determined by providing a valve to deliver a metered amount. The pressurized container or nebulizer may contain a solution or suspension of the active compound. Capsules and cartridges (made, for example, from gelatin) for use in an inhaler or insufflator may be formulated containing a powder mix of a compound of the application and a suitable powder base such as lactose or starch. The aerosol dosage forms can also take the form of a pump-atomizer.

Compositions suitable for buccal or sublingual administration include tablets, lozenges, and pastilles, wherein the active ingredient is formulated with a carrier such as sugar, acacia, tragacanth, or gelatin and glycerine. Compositions for rectal administration are conveniently in the form of suppositories containing a conventional suppository base such as cocoa butter.

Suppository forms of the compounds of the application are useful for vaginal, urethral and rectal administrations. Such suppositories will generally be constructed of a mixture of substances that is solid at room temperature but melts at body temperature. The substances commonly used to create such vehicles include but are not limited to theobroma oil (also known as cocoa butter), glycerinated gelatin, other glycerides, hydrogenated vegetable oils, mixtures of polyethylene glycols of various molecular weights and fatty acid esters of polyethylene glycol. See, for example: Remington's Pharmaceutical Sciences, 16th Ed., Mack Publishing, Easton, PA, 1980, pp. 1530-1533 for further discussion of suppository dosage forms.

Compounds of the application may also be coupled with soluble polymers as targetable drug carriers. Such polymers can include polyvinylpyrrolidone, pyran copolymer, polyhydroxypropylmethacrylamide-phenol, polyhydroxy-ethylaspartamide-phenol, or polyethyleneoxide-polylysine substituted with palmitoyl residues. Furthermore, compounds of the application may be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacrylates and crosslinked or amphipathic block copolymers of hydrogels.

In an embodiment, compounds of the application may be coupled with viral, non-viral or other vectors. Viral vectors may include retrovirus, lentivirus, adenovirus, herpesvirus, poxvirus, alphavirus, vaccinia virus or adeno-associated viruses. Non-viral vectors may include nanoparticles, cationic lipids, cationic polymers, metallic nanoparticles, nanorods, liposomes, micelles, microbubbles, cell-penetrating peptides, or lipospheres. Nanoparticles may include silica, lipid, carbohydrate, or other pharmaceutically acceptable polymers.

In some embodiments, depending on the mode of administration, the pharmaceutical composition will comprise from about 0.05 wt % to about 99 wt % or about 0.10 wt % to about 70 wt %, of the active ingredient (one or more compounds of the application), and from about 1 wt % to about 99.95 wt % or about 30 wt % to about 99.90 wt % of one or more pharmaceutically acceptable carriers, all percentages by weight being based on the total composition.

In an embodiment, a compound of the present application is administered with another therapeutic agent simultaneously or sequentially in separate unit dosage forms or together in a single unit dosage form. Accordingly, the present application provides a single unit dosage form comprising one or more compounds of the application (e.g. a compound of Formula (I)), an additional therapeutic agent, and a pharmaceutically acceptable carrier.

To be clear, in the above, the term “a compound” also includes embodiments wherein one or more compounds are referenced.

### III. Methods and Uses of the Application

The compounds of the application have been shown to be capable of inhibiting HPK1 activity. In an embodiment, the HPK1 is human HPK1, see for example, Hu, M. C. et. al.; Genes Dev. 10 (1): 2251-2264, 1996.

Accordingly, the present application includes a method for inhibiting HPK1, in a cell, either in a biological sample or in a patient, comprising administering an effective amount of one or more compounds of the application to the cell. The application also includes a use of one or more compounds of the application for inhibiting HPK1 in a cell as well as a use of one or more compounds of the application for the preparation of a medicament for inhibiting HPK1 in a cell. The application further includes one or more compounds of the application for use in inhibiting HPK1.

As the compounds of the application have been shown to be capable of inhibiting HPK1, the compounds of the application are useful for treating diseases, disorders or conditions by inhibiting HPK1. Therefore the compounds of the present application are useful as medicaments. Accordingly, the present application includes a compound of the application for use as a medicament.

The present application also includes a method of treating a disease, disorder or condition that is treatable by inhibiting HPK1 comprising administering a therapeutically effective amount of one or more compounds of the application to a subject in need thereof.

The present application also includes a use of one or more compounds of the application for treatment of a disease, disorder or condition that is treatable by inhibiting HPK1 as well as a use of one or more compounds of the application for the preparation of a medicament for treatment of a disease, disorder or condition that is treatable by inhibiting HPK1. The application further includes one or more compounds of the application for use in treating a disease, disorder or condition that is treatable by inhibiting HPK1.

In an embodiment, the disease, disorder or condition that is treatable by inhibiting HPK1 is a neoplastic disorder. Accordingly, the present application also includes a method of treating a neoplastic disorder comprising administering a therapeutically effective amount of one or more compounds of the application to a subject in need thereof. The present application also includes a use of one or more compounds of the application for treatment of a neoplastic disorder as well as a use of one or more compounds of the application for the preparation of a medicament for treatment of a neoplastic disorder. The application further includes one or more compounds of the application for use in treating a neoplastic disorder. In an embodiment, the treatment is in an amount effective to ameliorate at least one symptom of the neoplastic disorder, for example, reduced cell proliferation or reduced tumor mass, among others, in a subject in need of such treatment.

Compounds of the application have been demonstrated to inhibit HPK1 and hence cytokine release in immune derived cell (e.g. Jurkat-T cells). Therefore in another embodiment of the present application, the disease, disorder or condition that is treatable by inhibiting HPK1 is cancer. Accordingly, the present application also includes a method of treating cancer comprising administering a therapeutically effective amount of one or more compounds of the application to a subject in need thereof. The present application also includes a use of one or more compounds of the application for treatment of cancer as well as a use of one or more compounds of the application for the preparation of a medicament for treatment of cancer. The application further includes one or more compounds of the application for use in treating cancer. In an embodiment, the compound is administered for the prevention of cancer in a subject such as a mammal having a predisposition for cancer.

In an embodiment, the cancer is selected from hematologic cancers, breast cancers, ovarian cancers, lung cancers, melanomas, colon cancers and glioblastomas.

In an embodiment, the disease, disorder or condition that is treatable by inhibiting HPK1 is a disease, disorder or condition associated with an uncontrolled and/or abnormal cellular activity affected directly or indirectly by inhibiting HPK1. In another embodiment, the uncontrolled and/or abnormal cellular activity that is affected directly or indirectly by inhibiting HPK1 is proliferative activity in a cell. Accordingly, the application also includes a method of inhibiting proliferative activity in a cell, comprising administering an effective amount of one or more compounds of the application to the cell. The present application also includes a use of one or more compounds of the application for inhibition of proliferative activity in a cell as well as a use of one or more compounds of the application for the preparation of a medicament for inhibition of proliferative activity in a cell. The application further includes one or more compounds of the application for use in inhibiting proliferative activity in a cell by boosting immune cell function through HPK1 inhibition.

The present application also includes a method of inhibiting uncontrolled and/or abnormal cellular activities affected directly or indirectly by inhibiting HPK1 in a cell, either in a biological sample or in a subject, comprising administering an effective amount of one or more compounds of the application to the cell. The application also includes a use of one or more compounds of the application for inhibition of uncontrolled and/or abnormal cellular activities affected directly or indirectly by inhibiting HPK1 in a cell as well as a use of one or more compounds of the application for the preparation of a medicament for inhibition of uncontrolled and/or abnormal cellular activities affected directly or indirectly by inhibiting HPK1 in a cell. The application further includes one or more compounds of the application for use in inhibiting uncontrolled and/or abnormal cellular activities affected directly or indirectly by inhibiting HPK1 in a cell.

The present application also includes a method of treating a disease, disorder or condition that is treatable by inhibiting HPK1 comprising administering a therapeutically effective amount of one or more compounds of the application in combination with another known agent useful for treatment of a disease, disorder or condition that is treatable by inhibiting HPK1 to a subject in need thereof. The present application also includes a use of one or more compounds of the application in combination with another known agent useful for treatment of a disease, disorder or condition that is treatable by inhibiting HPK1 for treatment of a disease, disorder or condition that is treatable by inhibiting HPK1, as well as a use of one or more compounds of the application in combination with another known agent useful for treatment of a disease, disorder or condition that is treatable by inhibiting HPK1 for the preparation of a medicament for treatment of a disease, disorder or condition that is treatable by inhibiting HPK1. The application further includes one or more compounds of the application in combination with another known agent useful for treatment of a disease, disorder or condition that is treatable by inhibiting HPK1 for use in treating a disease, disorder or condition that is treatable by inhibiting HPK1. In an embodiment, the disease, disorder or condition treatable by inhibiting HPK1 is cancer.

In a further embodiment, the disease, disorder or condition that is treatable by inhibiting HPK1 is cancer and the one or more compounds of the application are administered in combination with one or more additional cancer treatments. In another embodiment, the additional cancer treatment is selected from radiotherapy, chemotherapy, targeted therapies such as antibody therapies and small molecule tyrosine-kinase inhibitors, immunotherapy, hormonal therapy and anti-angiogenic therapies.

When used in combination with other agents or therapies useful in treating diseases, disorders or conditions that are treatable by inhibiting HPK1, it is an embodiment that the compounds of the application are administered contemporaneously with those agents or therapies. As used herein, “contemporaneous administration” of two substances or therapies to a subject means providing each of the two substances or therapies so that they are both biologically active in the individual at the same time. The exact details of the administration will depend on the pharmacokinetics of the two substances or therapies in the presence of each other, and can include administering the two substances or therapies within a few hours of each other, or even administering one substance or therapy within 24 hours of administration of the other, if the pharmacokinetics are suitable. Design of suitable dosing regimens is routine for one skilled in the art. In particular embodiments, the substances or therapies will be administered substantially simultaneously, i.e., within minutes of each other, or in a single composition in the case of administration of two substances. It is a further embodiment of the present application that a combination of agents or therapies is administered to a subject in a non-contemporaneous fashion.

In an embodiment, the subject is a mammal. In an embodiment, the subject is human.

In the context of treating a disease, disorder or condition treatable by inhibition of HPK1, an effective amount is an amount that, for example, inhibits HPK1, compared to the inhibition without administration of the one or more compounds. Effective amounts may vary according to factors such as the disease state, age, sex and/or weight of the subject. The amount of a given compound that will correspond to such an amount will vary depending upon various factors, such as the given drug or compound, the pharmaceutical formulation, the route of administration, the type of condition, disease or disorder, the identity of the subject being treated, and the like, but can nevertheless be routinely determined by one skilled in the art. The effective amount is one that following treatment therewith manifests as an improvement in or reduction of any disease symptom. When the disease is cancer, amounts that are effective can cause a reduction in the number, growth rate, size and/or distribution of tumours.

The dosage of compounds of the application can vary depending on many factors such as the pharmacodynamic properties of the compound, the mode of administration, the age, health and weight of the recipient, the nature and extent of the symptoms, the frequency of the treatment and the type of concurrent treatment, if any, and the clearance rate of the compound in the subject to be treated. One of skill in the art can determine the appropriate dosage based on the above factors. Compounds of the application may be administered initially in a suitable dosage that may be adjusted as required, depending on the clinical response. Dosages will generally be selected to maintain a serum level of compounds of the application from about 0.01 μg/cc to about 1000 μg/cc, or about 0.1 μg/cc to about 100 μg/cc. As a representative example, oral dosages of one or more compounds of the application will range between about 1 mg per day to about 1000 mg per day for an adult, suitably about 1 mg per day to about 500 mg per day, more suitably about 1 mg per day to about 200 mg per day. For parenteral administration, a representative amount is from about 0.001 mg/kg to about 10 mg/kg, about 0.01 mg/kg to about 10 mg/kg, about 0.01 mg/kg to about 1 mg/kg or about 0.1 mg/kg to about 1 mg/kg will be administered. For oral administration, a representative amount is from about 0.001 mg/kg to about 10 mg/kg, about 0.1 mg/kg to about 10 mg/kg, about 0.01 mg/kg to about 1 mg/kg or about 0.1 mg/kg to about 1 mg/kg. For administration in suppository form, a representative amount is from about 0.1 mg/kg to about 10 mg/kg or about 0.1 mg/kg to about 1 mg/kg. In an embodiment of the application, compositions are formulated for oral administration and the compounds are suitably in the form of tablets containing 0.25, 0.5, 0.75, 1.0, 5.0, 10.0, 20.0, 25.0, 30.0, 40.0, 50.0, 60.0, 70.0, 75.0, 80.0, 90.0, 100.0, 150, 200, 250, 300, 350, 400, 450, 500, 550, 600, 650, 700, 750, 800, 850, 900, 950 or 1000 mg of active ingredient per tablet. Compounds of the application may be administered in a single daily, weekly or monthly dose or the total daily dose may be divided into two, three or four daily doses.

In an embodiment, the compounds of the application are administered at least once a week. However, in another embodiment, the compounds are administered to the subject from about one time per two weeks, three weeks or one month. In another embodiment, the compounds are administered about one time per week to about once daily. In another embodiment, the compounds are administered 2, 3, 4, 5 or 6 times daily. The length of the treatment period depends on a variety of factors, such as the severity of the disease, disorder or condition, the age of the subject, the concentration and/or the activity of the compounds of the application, and/or a combination thereof. It will also be appreciated that the effective dosage of the compound used for the treatment may increase or decrease over the course of a particular treatment regime. Changes in dosage may result and become apparent by standard diagnostic assays known in the art. In some instances, chronic administration is required. For example, the compounds are administered to the subject in an amount and for duration sufficient to treat the subject.

### IV. Methods of Preparing the Compounds of the Application

Compounds of the present application can be prepared by various synthetic processes. The choice of particular structural features and/or substituents may influence the selection of one process over another. The selection of a particular process to prepare a given compound of Formula (I) is within the purview of the person of skill in the art. Some starting materials for preparing compounds of the present application are available from commercial chemical sources. Other starting materials, for example as described below, are readily prepared from available precursors using straightforward transformations that are well known in the art. In the Schemes below showing the preparation of compounds of the application, all variables are as defined in Formula I, unless otherwise stated.

The compounds of Formula (I) generally can be prepared according to the processes illustrated in the Schemes below. In the structural formulae shown below the variables are as defined in Formula (I) unless otherwise stated. A person skilled in the art would appreciate that many of the reactions depicted in the Schemes below would be sensitive to oxygen and water and would know to perform the reaction under an anhydrous, inert atmosphere if needed. Reaction temperatures and times are presented for illustrative purposes only and may be varied to optimize yield as would be understood by a person skilled in the art.

Throughout the processes described herein it is to be understood that, where appropriate, suitable protecting groups will be added to, and subsequently removed from, the various reactants and intermediates in a manner that will be readily understood by one skilled in the art. Conventional procedures for using such protecting groups as well as examples of suitable protecting groups are described, for example, in “Protective Groups in Organic Synthesis”, T. W. Green, P. G. M. Wuts, Wiley-Interscience, New York, (1999).

It is also to be understood that a transformation of a group or substituent into another group or substituent by chemical manipulation can be conducted on any intermediate or final product on the synthetic path toward the final product, in which the possible type of transformation is limited only by inherent incompatibility of other functionalities carried by the molecule at that stage to the conditions or reagents employed in the transformation. Such inherent incompatibilities, and ways to circumvent them by carrying out appropriate transformations and synthetic steps in a suitable order, will be readily understood to one skilled in the art. Examples of transformations are given herein, and it is to be understood that the described transformations are not limited only to the generic groups or substituents for which the transformations are exemplified. References and descriptions of other suitable transformations are given in “Comprehensive Organic Transformations—A Guide to Functional Group Preparations” R. C. Larock, VHC Publishers, Inc. (1989). References and descriptions of other suitable reactions are described in textbooks of organic chemistry, for example, “Advanced Organic Chemistry”, March, 4th ed. McGraw Hill (1992) or, “Organic Synthesis”, Smith, McGraw Hill, (1994).

Techniques for purification of intermediates and final products include, for example, straight and reversed phase chromatography on column or rotating plate, recrystallisation, distillation and liquid-liquid or solid-liquid extraction, which will be readily understood by one skilled in the art.

The products of the processes of the application may be isolated according to known methods, for example, the compounds may be isolated by evaporation of the solvent, by filtration, centrifugation, chromatography or other suitable method.

Generally, the reactions described above are performed in a suitable inert organic solvent and at temperatures and for times that will optimize the yield of the desired compounds. Examples of suitable inert organic solvents include, but are not limited to, 2-propanol, dimethylformamide (DMF), 1,4-dioxane, methylene chloride, chloroform, tetrahydrofuran (THF), toluene, and the like.

The formation of a desired compound salt is achieved using standard techniques. For example, the neutral compound is treated with an acid or base in a suitable solvent and the formed salt is isolated by filtration, extraction or any other suitable method.

The formation of solvates of the compounds of the application will vary depending on the compound and the solvate. In general, solvates are formed by dissolving the compound in the appropriate solvent and isolating the solvate by cooling or using an antisolvent. The solvate is typically dried or azeotroped under ambient conditions. The selection of suitable conditions to form a particular solvate can be made by a person skilled in the art.

Prodrugs of the compounds of the present application may be, for example, conventional esters formed with available hydroxy, thiol, amino or carboxyl groups. For example, available hydroxy or amino groups may be acylated using an activated acid in the presence of a base, and optionally, in inert solvent (e.g. an acid chloride in pyridine).

One skilled in the art will recognize that where a reaction step of the present application is carried out in a variety of solvents or solvent systems, said reaction step may also be carried out in a mixture of the suitable solvents or solvent systems.

Accordingly, in an embodiment, the compounds of Formula (I) are prepared as shown in Scheme 1.

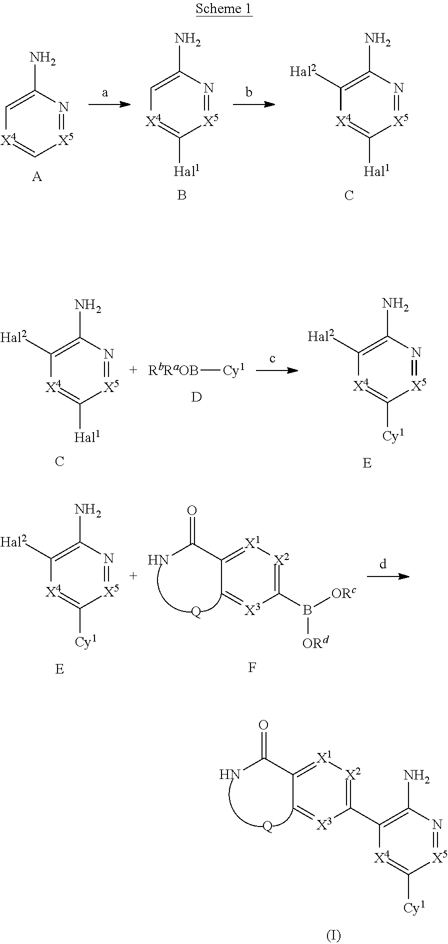

Accordingly, aminopyridine compounds of Formula (A) are mono-halogenated to form compounds of Formula (B), followed by subsequent halogenation to form intermediate compounds of Formula (C) wherein Hal¹ and Hal² are independently a halogen. Intermediate (C) is then coupled to boronic acid or boronic ester intermediate compounds of Formula (D) to form aminopyridine intermediate compounds of Formula (E). In an embodiment Hal¹ and Hal² are different halogens selected to have differing reactivity in the coupling reactions as would be known to those skilled in the art. In an embodiment Hal¹ and Hal² are Br and Cl respectively. In an embodiment Hal¹ and Hal² are I and Cl respectively. In an embodiment Hal¹ and Hal² are I and Br respectively. In an embodiment, R^{a} and R^{b} or R^{c} and R^{d} are all H. In an embodiment, R^{a} and R^{b} or R^{c} and R^{d} form a cycloalkyl ring. Compounds of Formula (E) are then coupled to boronic acid or ester compounds of Formula (F) to form the compounds of Formula (I). The variables Q, X¹, X², X³, X⁴, X⁵, Cy¹ are as defined in Formula (I). In an embodiment, both coupling reactions are performed under cross-coupling conditions, such as in the presence of a cross-coupling catalyst and in an inert solvent. In some embodiments the cross-coupling catalyst is a palladium catalyst. In an embodiment the halogenation conditions comprise a halogenation reagent, such as N-bromosuccinamide.

In a further embodiment, the compounds of Formula (I) are synthesized as shown in Scheme 2 by first coupling a boronic acid or a boronic ester compound of Formula (F) with a dihalogenated intermediate compound of Formula (G) by a Suzuki-Miyaura reaction to form the intermediate compound of Formula (H). Intermediate compound of Formula (H) is then reacted with a suitable boronic acid or boronic ester intermediate compound of Formula (D) to form a compound of Formula (I). The variables X¹, Q, X², X³, X⁴, X⁵, Cy¹ are as defined in Formula (I). In an embodiment Hal¹ and Hal² are different halogens selected to have differing reactivity in the coupling reactions as would be known to those skilled in the art. In an embodiment of the application, Hal¹ and Hal² are Br and I respectively. In an embodiment, R^{a} and R^{b} or R^{c} and R^{d} are all H. In an embodiment, R^{a} and R^{b} or R^{c} and R^{d} together form a cycloalkyl ring. In an embodiment, both coupling reactions are performed under cross-coupling conditions, such as in the presence of a cross-coupling catalyst and in an inert solvent. In some embodiments the cross-coupling catalyst is a palladium catalyst.

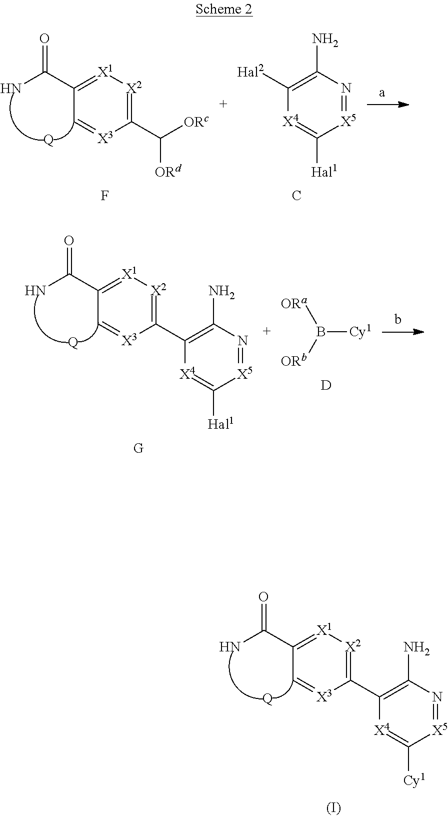

In a further embodiment, the compounds of Formula (C) are synthesized as shown in Scheme 3. Therefore, compounds of Formula (C) can be prepared by treating intermediate compound of Formula (A) with a halogenating reagent such as NBS to form mono-halogenated intermediate compound of Formula B which is then treated with a halogenating reagent such as N-iodosuccinimide (NIS) to provide the compounds of Formula C. In an embodiment Hal¹ and Hal² are different halogens selected to have differing reactivity in the coupling reactions as would be known to those skilled in the art. In an embodiment, Hal¹ and Hal² are, for example, Br and I respectively.

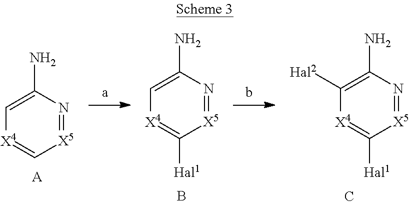

In an embodiment, the compounds of Formula (F) are synthesized as shown in Scheme 4. Therefore, compounds of Formula (F) are prepared by sequentially treating intermediate compounds of Formula (H) wherein Hal is a halogen with a suitable borylating agent such as 4,4,4′,4′,5,5,5′,5′-octamethyl-2,2′-bi(1,3,2-dioxaborolane in the presence of a suitable catalyst such as [1,1′-bis(diphenylphosphino)ferrocene]dichloropalladium(II) dichloromethane in an inert solvent such as 1,4-dixoane.

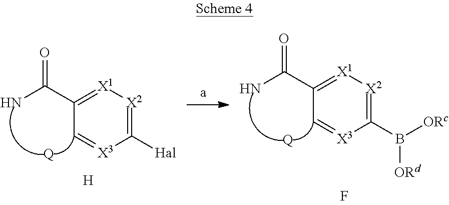

In an embodiment, as shown in Scheme 5, compounds of Formula (I) wherein Cy¹ is phenyl, and R¹¹ is

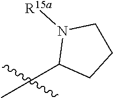

wherein R^{15a} is H or R¹⁵ are prepared by coupling a boronic acid or boronic ester compound of Formula (J) with a halogenated compound of Formula (K) under suitable conditions, for example, under Suzuki coupling conditions, to form compounds of Formula (I). In an embodiment, R¹ is an amino protecting group, for example, tert-butyloxycarbonyl (Boc) which is removed under suitable conditions, for example, with strong acids such as trifluoroacetic acid to form a compound of Formula (I) wherein R^{9a} is H. In an embodiment, Hal² is Br. In an embodiment, Rⁱ and R^{j} are both H. In an embodiment Rⁱ and R^{j} together, form a cycloalkyl ring. The variables Q, X¹, X², X³, X⁴, X⁵, Cy¹ and Cy² are as defined in Formula (I). In an embodiment, the compound of Formula I is the S-enantiomer

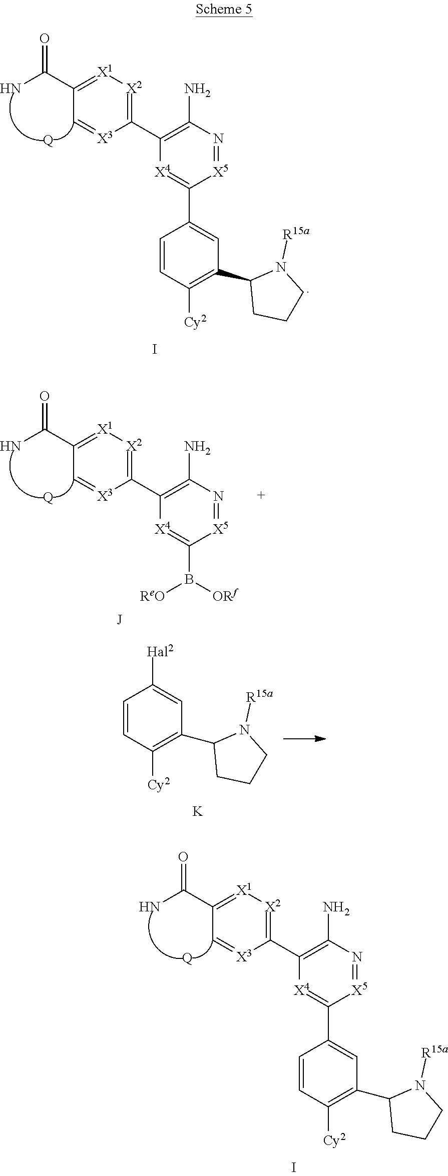

In an embodiment, as shown in Scheme 6, the intermediate compound of Formula (K) in Scheme 5 is synthesized by coupling dihalogenated ester compound of Formula (L) with halogened compound of Formula (M) under suitable coupling conditions such as in the presence of zinc to form halogenated ester compound of Formula (N). The halogenated ester compound of Formula (N) is then reduced under suitable reducing conditions such as in the presence of lithium aluminum hydride to form hydroxy compound of Formula (O) which is subsequently oxidized under suitable oxidizing conditions such as in the presence of manganese dioxide (MnO₂) to provide the halogenated aldehyde compound of Formula (P). The compound of Formula (P) is subsequently coupled with tert-butanesulfinamide (compound of Formula (Q)) to form the aldimine compound of Formula (R) which is further coupled with a (1,3-dioxan-2-ylethyl) (1,3-dioxan-2-ylethyl)magnesium bromide (compound of Formula (S)) under suitable Grignard reaction conditions to provide intermediate compound of Formula (T) which is cyclized under suitable cyclization conditions such as in the presence of trifluoric acid (TFA) and triethylsilane (EtSiH) to form the compound of Formula (K). In an embodiment, the tert-butanesulfinamide (compound of Formula Q) is S-tert-butanesulfinamide and the subsequent compound of Formula (R), (T), and (K) are S-enantiomers. In an embodiment Hal³, Hal⁴ and Hal⁵ are each halogens selected to work in the specific coupling reaction as would be known to those skilled in the art. In an embodiment Hal³ and Hal⁴ are Br and I respectively. In an embodiment, Hal⁵ is any suitable halogen. In an embodiment, Hal⁵ is I. The variables Cy¹ and Cy² are as defined in Formula I.

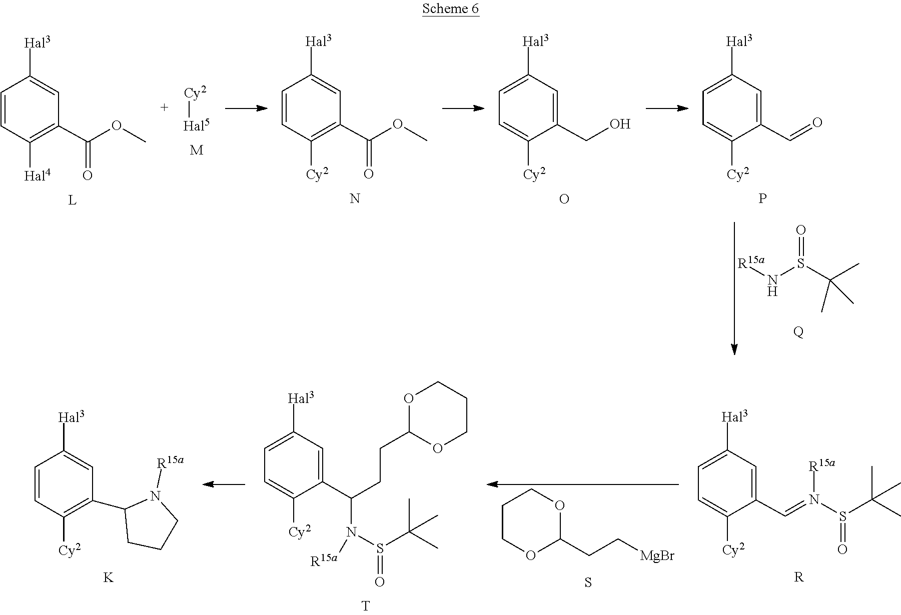

The schemes above are provided for illustration purposes. It will be understood by a person skilled in the art that the use of the most appropriate reagents may vary depend upon of the intermediates compounds of Formula (A)-(T), and that the most appropriate route may also be dependent upon the intermediates and the target compound of Formula (I).

Intermediates of compounds of Formula (A)-(t) are either commercially available or may be prepared using methods known in the art.

Generally the reactions described above are performed in a suitable inert organic solvent and at temperatures and for times that will optimize the yield of the desired compounds. Examples of suitable inert organic solvents include, but are not limited to, dimethylformamide (DMF), dioxane, methylene chloride, chloroform, tetrahydrofuran (THF), toluene, and the like.

Salts of the compounds of the application are generally formed by dissolving the neutral compound in an inert organic solvent and adding either the desired acid or base and isolating the resulting salt by either filtration or other known means.

The formation of solvates of the compounds of the application will vary depending on the compound and the solvate. In general, solvates are formed by dissolving the compound in the appropriate solvent and isolating the solvate by cooling or using an antisolvent. The solvate is typically dried or azeotroped under ambient conditions. The selection of suitable conditions to form a particular solvate can be made by a person skilled in the art. Examples of suitable solvents are ethanol, water and the like. When water is the solvent, the molecule is referred to as a “hydrate”.

Prodrugs of the compounds of the present application may be, for example, conventional esters formed with available hydroxy, thiol, amino or carboxyl groups. For example, available hydroxy or amino groups may be acylated using an activated acid in the presence of a base, and optionally, in inert solvent (e.g. an acid chloride in pyridine). Some common esters which have been utilized as prodrugs are phenyl esters, aliphatic (C1-C24) esters, acyloxymethyl esters, carbamates and amino acid esters.

Throughout the processes described herein it is to be understood that, where appropriate, suitable protecting groups will be added to, and subsequently removed from, the various reactants and intermediates in a manner that will be readily understood by one skilled in the art. Conventional procedures for using such protecting groups as well as examples of suitable protecting groups are described, for example, in “Protective Groups in Organic Synthesis”, T. W. Green, P. G. M. Wuts, Wiley-Interscience, New York, (1999). It is also to be understood that a transformation of a group or substituent into another group or substituent by chemical manipulation can be conducted on any intermediate or final product on the synthetic path toward the final product, in which the possible type of transformation is limited only by inherent incompatibility of other functionalities carried by the molecule at that stage to the conditions or reagents employed in the transformation. Such inherent incompatibilities, and ways to circumvent them by carrying out appropriate transformations and synthetic steps in a suitable order, will be readily understood to one skilled in the art. Examples of transformations are given herein, and it is to be understood that the described transformations are not limited only to the generic groups or substituents for which the transformations are exemplified. References and descriptions of other suitable transformations are given in “Comprehensive Organic Transformations—A Guide to Functional Group Preparations” R. C. Larock, VHC Publishers, Inc. (1989). References and descriptions of other suitable reactions are described in textbooks of organic chemistry, for example, “Advanced Organic Chemistry”, March, 4th ed. McGraw Hill (1992) or, “Organic Synthesis”, Smith, McGraw Hill, (1994). Techniques for purification of intermediates and final products include, for example, straight and reversed phase chromatography on column or rotating plate, recrystallisation, distillation and liquid-liquid or solid-liquid extraction, which will be readily understood by one skilled in the art.

The following non-limiting examples are illustrative of the present application.

### EXAMPLES

### A. Preparation of Exemplary Compounds of the Application

### Example 1: 6-(3-amino-5-fluoro-6-(4-(4-isopropylpiperazin-1-yl)phenyl)pyrazin-2-yl)-3,4-dihydroisoquinolin-1(2H)-one (I-1)

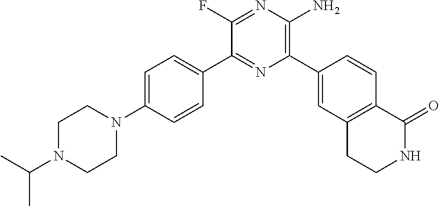 [SMILES: CC(C)N1CCN(c2ccc(-c3nc(-c4ccc5c(c4)CCNC5=O)c(N)nc3F)cc2)CC1]

### Step 1: 5-chloro-6-fluoropyrazin-2-amine

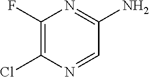

To a solution of 6-fluoropyrazin-2-amine (0.75 g, 6.6 mmol) in DMF (18 mL) at room temperature (RT) was added N-chlorosuccinimide (0.90 g, 6.9 mmol). The reaction was heated to 80° C. for 2 h. The reaction mixture was concentrated onto Celite® and purified by flash chromatography [0-35% EtOAc/hexanes] to afford the product (0.71 g, 72%) as an off-white solid. LCMS: [M−H]⁻=146.0.

### Step 2: 3-bromo-5-chloro-6-fluoropyrazin-2-amine

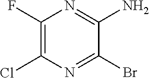

N-Bromosuccinimide (1.3 g, 7.1 mmol) was added to a solution of 5-chloro-6-fluoropyrazin-2-amine (0.70 g, 4.7 mmol) in DCM (15 mL) at RT. The reaction was stirred for 18 h at RT. The reaction mixture was concentrated onto Celite® and purified by flash chromatography (0-30% EtOAc/hexanes) to afford the product (0.99 g, 92%). LCMS: [M−H]⁻=224.3.

### Step 3: 6-(3-amino-6-chloro-5-fluoropyrazin-2-yl)-3,4-dihydroisoquinolin-1(2H)-one

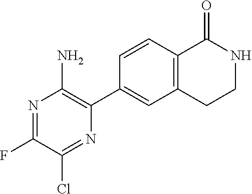 [SMILES: Nc1nc(F)c(Cl)nc1-c1ccc2c(c1)CCNC2=O]

A 30 mL was charged with 3,4-dihydro-1(2H)-isoquinolinone-6-boronic acid pinacol ester (0.15 g, 0.54 mmol), 3-bromo-5-chloro-6-fluoropyrazin-2-amine (0.081 g, 0.36 mmol) and [1,12-bis(diphenylphosphino)ferrocene]dichloropalladium (II) DCM complex (0.020 g, 0.025 mmol). The vial was sealed with a cap and septum and the reaction vessel was evacuated and backfilled with nitrogen. 1,4-Dioxane (5 mL) and 2 M aqueous Na₂CO₃ (0.54 mL, 1.1 mmol) were added and the reaction vessel was evacuated and backfilled with nitrogen an additional time. The reaction mixture was heated at 90° C. for 18 h in an aluminum block. The reaction mixture was concentrated onto Celite® and purified by flash chromatography (0.5-9.5% MeOH/DCM+0.5% NH₄OH) to afford the product (0.061 g, 58%). LCMS: [M+H]⁺=293.3.

### Step 4: 6-(3-amino-5-fluoro-6-(4-(4-isopropylpiperazin-1-yl)phenyl)pyrazin-2-yl)-3,4-dihydroisoquinolin-1(2H)-one

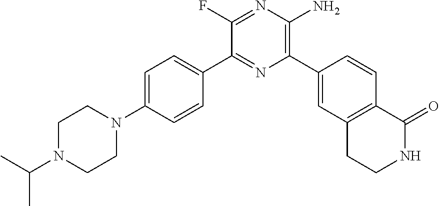 [SMILES: CC(C)N1CCN(c2ccc(-c3nc(-c4ccc5c(c4)CCNC5=O)c(N)nc3F)cc2)CC1]

A 30 mL vial was charged with 4-(4-isopropylpiperazinyl)phenylboronic acid, pinacol ester (0.10 g, 0.31 mmol), 6-(3-amino-6-chloro-5-fluoropyrazin-2-yl)-3,4-dihydroisoquinolin-1(2H)-one (0.061 g, 0.21 mmol) and XPhos Pd G2 (0.016 g, 0.021 mmol). The vial was sealed with a cap and septum and the reaction vessel was evacuated and backfilled with nitrogen. 1,4-Dioxane (3 mL) and aqueous K₃PO₄ (0.40 mL of a 1.3 M solution, 0.52 mmol) were added and the reaction vessel was evacuated and backfilled with nitrogen an additional time. The reaction mixture was heated at 90° C. for 18 h in an aluminum block. The reaction mixture was concentrated onto Celite® and purified by flash chromatography (0.5-9.5% DCM/MeOH+0.5% NH₄OH). The product containing fractions were concentrated and further purified by reverse phase chromatography (Biotage SNAP C18; 5-60% MeCN/water+0.1% Formic Acid). Isolation of the title compound was achieved by a catch and release procedure using Biotage SCX2 silica gel to afford the title compound (4.0 mg, 4%). ¹H NMR (500 MHz, DMSO-d₆) δ 7.9-8.0 (m, 2H), 7.75 (d, J=7.9 Hz, 2H), 7.7-7.7 (m, 1H), 7.65 (s, 1H), 7.01 (d, J=9.0 Hz, 2H), 6.71 (s, 2H), 3.2-3.2 (m, 4H), 2.99 (t, J=6.5 Hz, 2H), 2.7-2.7 (m, 1H), 2.6-2.6 (m, 4H), 1.01 (d, J=6.5 Hz, 6H); LCMS: [M+H]⁺=461.6.

### Example 2: 6-(3-amino-5-fluoro-6-(4-(4-isopropylpiperazin-1-yl)phenyl)pyrazin-2-yl)-7-fluoro-3,4-dihydroisoquinolin-1(2H)-one (I-2)

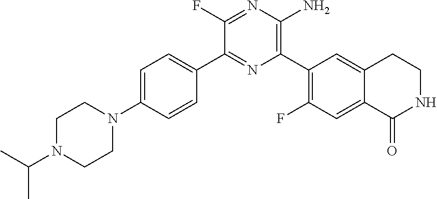 [SMILES: CC(C)N1CCN(c2ccc(-c3nc(-c4cc5c(cc4F)C(=O)NCC5)c(N)nc3F)cc2)CC1]

### Step 1: 5-chloro-6-fluoropyrazin-2-amine

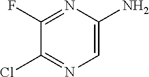

To a solution of 6-fluoropyrazin-2-amine (0.250 g, 2.211 mmol) in N,N-dimethylformamide (DMF) (6 mL) at RT was added N-chlorosuccinimide (0.301 g, 2.26 mmol). The reaction was heated to 80° C. for 2 h. The DMF was removed in vaccuo and the residue was dissolved in CH₂Cl₂ and concentrated onto Celite®. Flash chromatography (0-35% EtOAc/hexanes) afforded the major regioisomer 5-chloro-6-fluoropyrazin-2-amine (0.708 g, 72.4% yield). LCMS [M−H]⁻=146.00.

### Step 2: 3-bromo-5-chloro-6-fluoropyrazin-2-amine

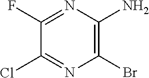

To a solution of 5-chloro-6-fluoropyrazin-2-amine (0.70 g, 4.74 mmol) in CH₂Cl₂ (15 ml) at room temperature was added N-bromosuccinimide (1.267 g, 7.12 mmol). The reaction was stirred for 19 h. The reaction mixture was concentrated onto Celite®. Flash chromatography (0-30% EtOAc/hexanes) afforded the product. LCMS [M+H]⁺=226.02.

### Step 3: 8-fluoro-6-(4,4,5,5-tetramethyl-1,3,2-dioxaborolan-2-yl)-3,4-dihydroiso-quinolin-1(2H)-one

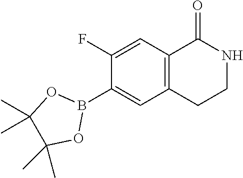 [SMILES: CC1(C)OB(c2cc3c(cc2F)C(=O)NCC3)OC1(C)C]

To 6-bromo-5-fluoro-3,4-dihydroisoquinolin-1(2H)-one (50 mg, 0.205 mmol, 1 eq), bis(pinacolato)diboron (58.2 mg, 0.229 mmol, 1.1 eq), KOAc (61.3 mg, 0.625 mmol, 3 eq) and [1,12-bis(diphenylphosphino)ferrocene]dichloropalladium(II) (7.62 mg, 10.41 μmol, 0.05 eq) in 1,4-dioxane (2.5 mL) was added under Ar. The mixture was heated in a microwave for 2 h at 100° C. The LCMS showed less than 10% of starting material. The reaction mixture was used in the next steps without purification. LCMS: [M+H]⁺=292.0

### Step 4: 6-(3-amino-6-chloro-5-fluoropyrazin-2-yl)-7-fluoro-3,4-dihydroisoquinolin-1(2H)-one

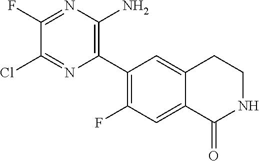 [SMILES: Nc1nc(F)c(Cl)nc1-c1cc2c(cc1F)C(=O)NCC2]

A procedure similar to Example 1, Step 3 using 7-fluoro-6-(4,4,5,5-tetramethyl-1,3,2-dioxaborolan-2-yl)-3,4-dihydroisoquinolin-1(2H)-one (0.579 g, 1.99 mmol), 3-bromo-5-chloro-6-fluoropyrazin-2-amine (0.300 g, 1.33 mmol), [1,12-bis(diphenylphosphino)ferrocene]dichloropalladium(II), DCM complex (0.076 g, 0.093 mmol), 1,4-dioxane (8 mL) and aqueous Na₂CO₃ (1.99 mL of a 2 M solution, 3.97 mmol) afforded the product (129 mg, 31%). LCMS: [M+H]⁺=311.37.

### Step 5: 6-(3-amino-5-fluoro-6-(4-(4-isopropylpiperazin-1-yl)phenyl)pyrazin-2-yl)-7-fluoro-3,4-dihydroisoquinolin-1(2H)-one (I-2)

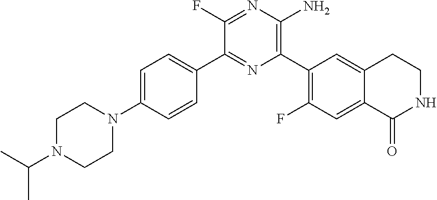 [SMILES: CC(C)N1CCN(c2ccc(-c3nc(-c4cc5c(cc4F)C(=O)NCC5)c(N)nc3F)cc2)CC1]

A procedure similar to Example 1, Step 4 using 4-(4-isopropylpiperazinyl)phenylboronic acid, pinacol ester (0.080 g, 0.241 mmol), 6-(3-amino-6-chloro-5-fluoropyrazin-2-yl)-7-fluoro-3,4-dihydroisoquinolin-1(2H)-one (0.050 g, 0.161 mmol) and XPhos Pd G2 (0.013 g, 0.016 mmol), 1,4-dioxane (3 mL) and aqueous K₃PO₄ (0.309 mL of a 1.3 M solution, 0.402 mmol) afforded the title compound (61 mg, 79% yield) as a yellow powder. ¹H NMR (500 MHz, DMSO-d₆) δ 8.15 (br s, 1H), 7.70 (d, J=7.8 Hz, 2H), 7.64 (d, J=10.1 Hz, 1H), 7.51 (d, J=6.8 Hz, 1H), 7.00 (d, J=9.0 Hz, 2H), 6.67 (s, 2H), 3.42 (br dd, J=6.6, 2.7 Hz, 3H), 3.2-3.2 (m, 4H), 2.96 (br t, J=6.4 Hz, 2H), 2.7-2.7 (m, 1H), 2.6-2.6 (m, 4H), 1.01 (d, J=6.5 Hz, 6H); LCMS: [M+H]⁺=479.5.

### Example 3: 6-(3-amino-5-fluoro-6-(4-((1R,5S)-3-methyl-3-azabicyclo[3.1.0]hexan-1-yl)phenyl)pyrazin-2-yl)-7-fluoro-3,4-dihydroisoquinolin-1(2H)-one (I-3)

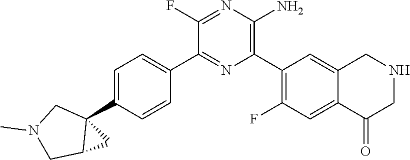 [SMILES: CN1C[C@H]2C[C@@]2(c2ccc(-c3nc(-c4cc5c(cc4F)C(=O)CNC5)c(N)nc3F)cc2)C1]

### Step 1: 5-chloro-6-fluoropyrazin-2-amine

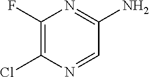

To a solution of 6-fluoropyrazin-2-amine (0.75 g, 6.6 mmol) in DMF (18 mL) at room temperature (RT) was added N-chlorosuccinimide (0.90 g, 6.9 mmol). The reaction was heated to 80° C. for 2 h. The reaction mixture was concentrated onto Celite® and purified by flash chromatography (0-35% EtOAc/hexanes) to afford the product (0.71 g, 72%) as a colorless solid. LCMS: [M−H]⁻146.0.

### Step 2: 3-bromo-5-chloro-6-fluoropyrazin-2-amine

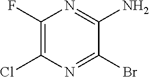

N-Bromosuccinimide (1.3 g, 7.1 mmol) was added to a solution of 5-chloro-6-fluoropyrazin-2-amine (0.70 g, 4.7 mmol) in DCM (15 mL) at RT. The reaction was stirred for 18 h at RT, concentrated onto Celite® and purified by flash chromatography (0-30% EtOAc/hexanes) to afford the product (0.99 g, 92%). LCMS: [M−H]⁻=224.3.

### Step 3: 6-(3-amino-6-chloro-5-fluoropyrazin-2-yl)-7-fluoro-3,4-dihydroisoquinolin-1(2H)-one

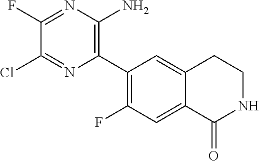 [SMILES: Nc1nc(F)c(Cl)nc1-c1cc2c(cc1F)C(=O)NCC2]

A 30 mL was charged with 7-fluoro-6-(4,4,5,5-tetramethyl-1,3,2-dioxaborolan-2-yl)-3,4-dihydroisoquinolin-1(2H)-one (0.58 g, 2.0 mmol), 3-bromo-5-chloro-6-fluoropyrazin-2-amine (0.30 g, 1.3 mmol) and [1,12-bis(diphenylphosphino)ferrocene]dichloropalladium (II) DCM complex (0.076 g, 0.093 mmol). The vial was sealed with a cap and septum and the reaction vessel was evacuated and backfilled with nitrogen. 1,4-Dioxane (8 mL) and 2 M aqueous Na₂CO₃ (2.0 mL, 4.0 mmol) were added and the reaction vessel was evacuated and backfilled with nitrogen an additional time. The reaction mixture was heated at 90° C. for 18 h in an aluminum block. The reaction mixture was concentrated onto Celite® and purified by flash chromatography (0.5-9.5% MeOH/DCM+0.5% NH₄OH) to afford the product (0.12 g, 28%). LCMS: [M+H]⁺=311.3.

### Step 4: (1R,5S)-1-(4-bromophenyl)-3-methyl-3-azabicyclo[3.1.0]hexane

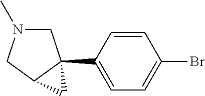

To a solution of (1R,5S)-1-(4-bromophenyl)-3-azabicyclo[3.1.0]hexane (0.50 g, 2.1 mmol) in 1:1 MeOH:THF (10 mL) was added formaldehyde (37% wt, 0.23 mL, 3.2 mmol) followed by NaBH(OAc)₃ (0.67 g, 3.2 mmol). The reaction was stirred at RT for 40 h. The volatiles were removed in vaccuo and the residue was partitioned between 1 M aqueous KOH (15 mL) and DCM (15 mL). The layers were separated and the aqueous layer was extracted with additional portions of DCM. The combined extracts were dried over MgSO₄ and concentrated to dryness to afford the product (0.50 g, 95%) that was used in the next step without purification. LCMS: [M+H]⁺=252.2.

### Step 5: 6-(3-amino-5-fluoro-6-(4-((1R,5S)-3-methyl-3-azabicyclo[3.1.0]hexan-1-yl)phenyl)pyrazin-2-yl)-7-fluoro-3,4-dihydroisoquinolin-1(2H)-one

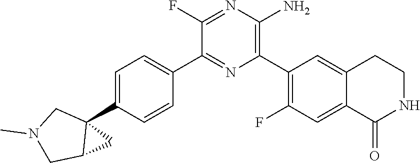 [SMILES: CN1C[C@H]2C[C@@]2(c2ccc(-c3nc(-c4cc5c(cc4F)C(=O)NCC5)c(N)nc3F)cc2)C1]

A stock solution of (5-amino-3-fluoro-6-(7-fluoro-1-oxo-1,2,3,4-tetrahydroisoquinolin-6-yl)pyrazin-2-yl)boronic acid was prepared as follows: A 30 mL vial was charged with 6-(3-amino-6-chloro-5-fluoropyrazin-2-yl)-7-fluoro-3,4-dihydroisoquinolin-1(2H)-one (0.24 g, 0.77 mmol), bis(pinacolato)diboron (0.29 g, 1.2 mmol), XPhos Pd G2 (0.091 g, 0.12 mmol) and KOAc (0.19 g, 1.9 mmol). The vial was sealed with a cap and septum and evacuated and back filled with nitrogen gas. 1,4-Dioxane (8 mL) was added and the reaction vial was evacuated and flushed with N₂. The reaction was heated to 90° C. in an aluminum block for 1 h. After cooling to RT, 2.0 mL of this stock solution was transferred to a sealed 30 mL vial that was charged with (1R,5S)-1-(4-bromophenyl)-3-methyl-3-azabicyclo[3.1.0]hexane (0.047 g, 0.19 mmol) and XPhos Pd G2 (0.015 g, 0.019 mmol). Aqueous K₃PO₄ (0.36 mL of a 1.3 M solution, 0.47 mmol) was added via syringe and the reaction vessel was evacuated and backfilled with nitrogen. The reaction mixture was heated at 90° C. for 18 h in an aluminum block. The reaction mixture was concentrated onto Celite® and purified by flash chromatography (0.5-9.5% MeOH/DCM+0.5% NH₄OH). The product containing fractions were concentrated and further purified by reverse phase chromatography (Biotage SNAP C18; 5-45% MeCN/water+0.1% Formic Acid). Isolation of the title compound was achieved by a catch and release procedure using Biotage SCX2 silica gel to afford the product (0.020 g, 24%). ¹H NMR (500 MHz, DMSO-d₆) δ 8.08 (br s, 1H), 7.67 (d, J=7.3 Hz, 2H), 7.57 (d, J=10.1 Hz, 1H), 7.45 (d, J=6.8 Hz, 1H), 7.14 (d, J=8.6 Hz, 2H), 6.76 (s, 2H), 3.35 (br dd, J=6.6, 2.4 Hz, 4H), 3.20 (br d, J=8.4 Hz, 1H), 2.9-2.9 (m, 3H), 2.33 (br d, J=5.9 Hz, 1H), 2.24 (s, 3H), 1.75 (td, J=7.8, 3.9 Hz, 1H), 1.30 (t, J=4.1 Hz, 1H), 0.71 (dd, J=7.9, 3.8 Hz, 1H); LCMS: [M+H]⁺=448.6.

### Example 4: 6-(3-amino-5-fluoro-6-(4-((1R,5S)-3-isopropyl-3-azabicyclo[3.1.0]hexan-1-yl)phenyl)pyrazin-2-yl)-7-fluoro-3,4-dihydroisoquinolin-1(2H)-one (I-4)

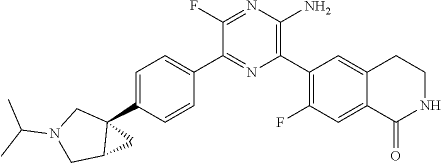 [SMILES: CC(C)N1C[C@H]2C[C@@]2(c2ccc(-c3nc(-c4cc5c(cc4F)C(=O)NCC5)c(N)nc3F)cc2)C1]

### Step 1: (1R,5S)-1-(4-bromophenyl)-3-isopropyl-3-azabicyclo[3.1.0]hexane

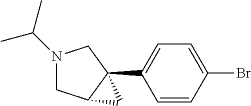 [SMILES: CC(C)N1C[C@H]2C[C@@]2(c2ccc(Br)cc2)C1]

To a solution of (1R,5S)-1-(4-bromophenyl)-3-azabicyclo[3.1.0]hexane (0.500 g, 2.100 mmol) in MeOH/THF (5 mL each) was added acetone (0.231 mL, 3.15 mmol) and acetic acid (0.012 mL, 0.210 mmol) followed by NaBH(OAc)₃ (0.668 g, 3.15 mmol). The reaction was stirred at RT for 40 h. The volatiles were removed in vacuo and the residue was partitioned between KOH (1N) and CH₂Cl₂. The layers were separated and the aqueous layer was extracted with additional CH₂Cl₂. The combined extracts were dried over MgSO₄ and concentrated to dryness to afford the product (2.10 mmol, 100%) as an amber oil. LCMS: [M+H]⁺=280.29.

### Step 2: 6-(3-amino-5-fluoro-6-(4,4,5,5-tetramethyl-1,3,2-dioxaborolan-2-yl)pyrazin-2-yl)-7-fluoro-3,4-dihydroisoquinolin-1(2H)-one

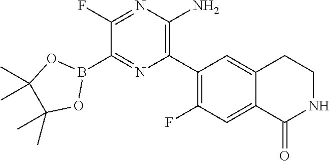 [SMILES: CC1(C)OB(c2nc(-c3cc4c(cc3F)C(=O)NCC4)c(N)nc2F)OC1(C)C]

A 30 mL vial was charged with 6-(3-amino-6-chloro-5-fluoropyrazin-2-yl)-7-fluoro-3,4-dihydroisoquinolin-1(2H)-one (0.240 g, 0.772 mmol), bis(pinacolato)diboron (0.294 g, 1.16 mmol), XPhos Pd G2 (0.091 g, 0.116 mmol) and KOAc (0.190 g, 1.93 mmol). The vial was sealed with a cap and septum and evacuated and back filled with nitrogen gas. 1,4-Dioxane (8 ml) was added and the reaction vial was evacuated and refilled again with N₂. The reaction was heated to 90° C. in an aluminum block for 1 h. The reaction was allowed to cool to RT and carried forward into subsequent reactions as a solution without isolation of the product LCMS [M+H]+=318.37.

### Step 3: 6-(3-amino-5-fluoro-6-(4-((1R,5S)-3-isopropyl-3-azabicyclo[3.1.0]hexan-1-yl)phenyl)pyrazin-2-yl)-7-fluoro-3,4-dihydroisoquinolin-1(2H)-one

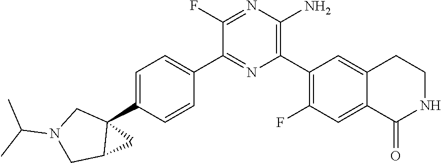 [SMILES: CC(C)N1C[C@H]2C[C@@]2(c2ccc(-c3nc(-c4cc5c(cc4F)C(=O)NCC5)c(N)nc3F)cc2)C1]

A procedure analogous to Example 3, Step 5 using (1R,5S)-1-(4-bromophenyl)-3-isopropyl-3-azabicyclo[3.1.0]hexane (0.052 g, 0.186 mmol), XPhos Pd G2 (0.015 g, 0.019 mmol) 6-(3-amino-5-fluoro-6-(4,4,5,5-tetramethyl-1,3,2-dioxaborolan-2-yl)pyrazin-2-yl)-7-fluoro-3,4-dihydroisoquinolin-1(2H)-one (0.075 g, 0.186 mmol, freshly prepared in Example 4, Step 2 above) in 1,4-dioxane (2.0 ml/L) aqueous K₃PO₄ (0.359 mL, 0.466 mmol) afforded the title compound (24 mg, 27%). ¹H NMR (500 MHz, DMSO-d₆) δ 8.08 (br s, 1H), 7.66 (d, J=7.5 Hz, 2H), 7.58 (d, J=10.3 Hz, 1H), 7.45 (d, J=6.7 Hz, 1H), 7.15 (d, J=8.4 Hz, 2H), 6.76 (s, 2H), 3.35 (br dd, J=6.2, 2.2 Hz, 4H), 2.98 (d, J=8.6 Hz, 1H), 2.88 (br t, J=6.4 Hz, 2H), 2.51 (br d, J=8.3 Hz, 2H,), 2.4-2.4 (m, 2H), 1.76 (td, J=7.8, 3.9 Hz, 1H), 1.25 (t, J=3.9 Hz, 1H), 0.95 (dd, J=14.7, 6.3 Hz, 6H), 0.69 (dd, J=7.8, 3.5 Hz, 1H); LCMS [M+H]⁺=476.5.

### Example 5: 6-(3-amino-5-fluoro-6-(4-((1S,5R)-3-methyl-3-azabicyclo[3.1.0]hexan-1-yl)phenyl)pyrazin-2-yl)-7-fluoro-3,4-dihydroisoquinolin-1(2H)-one (I-5)

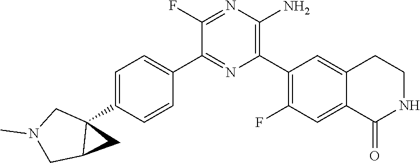 [SMILES: CN1C[C@@H]2C[C@]2(c2ccc(-c3nc(-c4cc5c(cc4F)C(=O)NCC5)c(N)nc3F)cc2)C1]

A procedure analogous to Example 3, Step 5 using (1S,5R)-1-(4-bromophenyl)-3-methyl-3-azabicyclo[3.1.0]hexane (0.047 g, 0.186 mmol) and XPhos Pd G2 (0.015 g, 0.019 mmol), 6-(3-amino-5-fluoro-6-(4,4,5,5-tetramethyl-1,3,2-dioxaborolan-2-yl)pyrazin-2-yl)-7-fluoro-3,4-dihydroisoquinolin-1(2H)-one (0.075 g, 0.186 mmol) in 1,4-dioxane (2.0 mL) and aqueous K₃PO₄ (0.359 mL of a 1.3 M solution, 0.466 mmol) afforded the title compound. ¹H NMR (500 MHz, DMSO-d₆) δ 8.08 (br s, 1H), 7.67 (d, J=7.3 Hz, 2H), 7.57 (d, J=10.1 Hz, 1H), 7.45 (d, J=6.8 Hz, 1H), 7.14 (d, J=8.6 Hz, 2H), 6.7-6.8 (m, 2H), 3.3-3.4 (m, 4H), 3.19 (d, J=8.4 Hz, 1H), 2.9-2.9 (m, 3H), 2.32 (dd, J=8.6, 3.4 Hz, 1H), 1.74 (td, J=7.8, 3.9 Hz, 1H), 1.30 (t, J=4.1 Hz, 1H), 0.71 (dd, J=7.9, 3.8 Hz, 1H); LCMS: [M+H]⁺=448.5.

### Example 6: 6-(3-amino-5-fluoro-6-(4-((1S,5R)-3-isopropyl-3-azabicyclo[3.1.0]hexan-1-yl)phenyl)pyrazin-2-yl)-7-fluoro-3,4-dihydroisoquinolin-1(2H)-one (I-6)

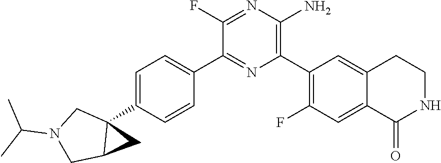 [SMILES: CC(C)N1C[C@@H]2C[C@]2(c2ccc(-c3nc(-c4cc5c(cc4F)C(=O)NCC5)c(N)nc3F)cc2)C1]

### Step 1: (1S,5R)-1-(4-bromophenyl)-3-isopropyl-3-azabicyclo[3.1.0]hexane

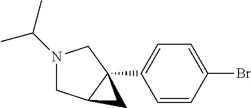 [SMILES: CC(C)N1C[C@@H]2C[C@]2(c2ccc(Br)cc2)C1]

A procedure similar to Example 4, Step 1 using (1S,5R)-1-(4-bromophenyl)-3-azabicyclo[3.1.0]hexane (0.500 g, 2.100 mmol) in MeOH/THF (5 mL each), acetone (0.231 mL, 3.15 mmol) and AcOH (0.012 mL, 0.210 mmol) followed by NaBH(OAc)₃ (0.668 g, 3.15 mmol) afforded the product (665 mg, quantitative yield) as an amber oil that was used in the next step without further purification. LCMS: [M+H]+=280.29.

### Step 2: 6-(3-amino-5-fluoro-6-(4-((1S,5R)-3-isopropyl-3-azabicyclo[3.1.0]hexan-1-yl)phenyl)pyrazin-2-yl)-7-fluoro-3,4-dihydroisoquinolin-1(2H)-one (I-6)

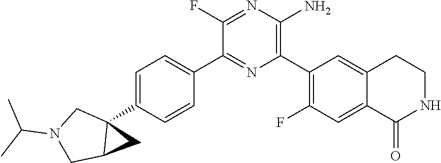 [SMILES: CC(C)N1C[C@@H]2C[C@]2(c2ccc(-c3nc(-c4cc5c(cc4F)C(=O)NCC5)c(N)nc3F)cc2)C1]

A procedure similar to Example 3, Step 5 using (1S,5R)-1-(4-bromophenyl)-3-isopropyl-3-azabicyclo[3.1.0]hexane (0.052 g, 0.186 mmol), XPhos Pd G2 (0.015 g, 0.019 mmol) and 6-(3-amino-5-fluoro-6-(4,4,5,5-tetramethyl-1,3,2-dioxaborolan-2-yl)pyrazin-2-yl)-7-fluoro-3,4-dihydroisoquinolin-1(2H)-one (0.075 g, 0.186 mmol in 1,4-dioxane (2.0 mL) and aqueous K₃PO₄ (0.359 mL of a 1.3 M solution, 0.466 mmol) afforded the title compound (9 mg, 10%) as an off-white residue. ¹H NMR (500 MHz, DMSO-d₆) δ 8.08 (br s, 1H), 7.66 (d, J=7.3 Hz, 2H), 7.57 (d, J=10.1 Hz, 1H), 7.45 (d, J=6.8 Hz, 1H), 7.15 (d, J=8.4 Hz, 2H), 6.76 (s, 2H), 3.35 (br dd, J=6.4, 2.5 Hz, 4H), 2.98 (d, J=8.6 Hz, 1H), 2.88 (br t, J=6.5 Hz, 2H), 2.51 (br d, J=8.3 Hz, 2H), 2.4-2.4 (m, 2H), 1.76 (td, 1H, J=7.8, 3.9 Hz), 1.25 (t, J=3.9 Hz, 1H), 0.95 (dd, 6H, J=14.7, 6.2 Hz), 0.68 (dd, J=7.9, 3.6 Hz, 1H); LCMS: [M+H]⁺=476.5.

### Example 7: 6-(3-amino-5-chloro-6-(4-(4-isopropylpiperazin-1-yl)phenyl)pyrazin-2-yl)-3,4-dihydroisoquinolin-1(2H)-one (I-7)

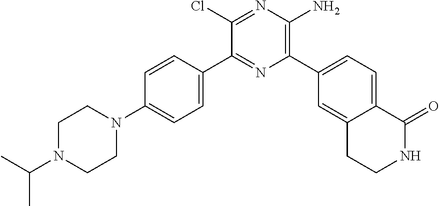 [SMILES: CC(C)N1CCN(c2ccc(-c3nc(-c4ccc5c(c4)CCNC5=O)c(N)nc3Cl)cc2)CC1]

### Step 1: 5-bromo-6-chloro-3-iodopyrazin-2-amine

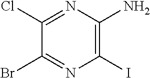

To a solution of 5-bromo-6-chloropyrazin-2-amine (1.6 g, 7.68 mmol) in AcOH (14 mL) was added N-iodosuccinimide (2.245 g, 9.98 mmol). TFA (20 drops) was then added, and the mixture was stirred at 50° C. for 3 h. The mixture was allowed to cool to RT, then mixed with water (60 mL). The resulting precipitate was filtered and the filter cake was washed with water (3×). The solid was then taken up in water (60 mL) and basified with 28-30% NH₄OH solution. The suspension was filtered, the filter cake was washed with water (3×) and dried under high vac o/n to get the product (2.306 g, 90%) as a pale yellow solid. LCMS: [M+H]⁺=334.28.

### Step 2: 6-(3-amino-6-bromo-5-chloropyrazin-2-yl)-3,4-dihydroisoquinolin-1(2H)-one

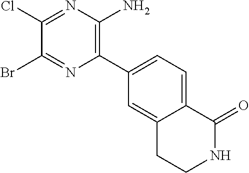 [SMILES: Nc1nc(Cl)c(Br)nc1-c1ccc2c(c1)CCNC2=O]

Water (12 mL) was added to a degassed suspension of 5-bromo-6-chloro-3-iodopyrazin-2-amine (698 mg, 2.09 mmol), 3,4-dihydro-1(2H)-isoquinolinone-6-boronic acid pinacol ester (500 mg, 1.83 mmol), Na₂CO₃H₂O (477 mg, 3.84 mmol) and bis(triphenylphosphine)palladium(II) dichloride (154 mg, 0.220 mmol) in CH₃CN (28 mL) and the reaction mixture was heated in a microwave reactor at 80° C. for 1 h. The mixture was mixed with water and EA, the organic phase was separated, the aqueous phase was extracted with CHCL₃/i-PrOH 4:1 mixture (4×). The combined organic phase was concentrated onto Celite® and purified by silica gel chromatography (12 g column), eluting with CH₂Cl₂ containing 0-2% MeOH and 0-0.2% NH₄OH. The appropriate fractions were combined and concentrated to afford the desired product as a beige solid. (447 mg, 69% yield). LCMS: [M+H]⁺=355.40.

### Step 3: 6-(3-amino-5-chloro-6-(4-(4-isopropylpiperazin-1-yl)phenyl)pyrazin-2-yl)-3,4-dihydroisoquinolin-1(2H)-one (I-7)

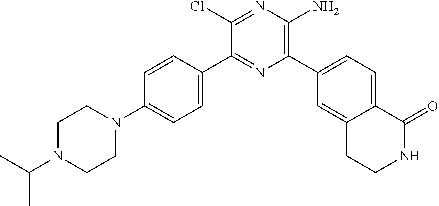 [SMILES: CC(C)N1CCN(c2ccc(-c3nc(-c4ccc5c(c4)CCNC5=O)c(N)nc3Cl)cc2)CC1]

A microwave vial containing a mixture of 6-(3-amino-6-bromo-5-chloropyrazin-2-yl)-3,4-dihydroisoquinolin-1(2H)-one (50 mg, 0.141 mmol), 4-(4-Isopropylpiperazinyl)phenylboronic acid, pinacol ester (65.4 mg, 0.198 mmol), [1,12-bis(diphenylphosphino)ferrocene]dichloropalladium(II) (15.52 mg, 0.021 mmol) and Cs₂CO₃ (138 mg, 0.424 mmol) in DME (3.5 mL) was degassed with argon. Water (1 mL) was added and the mixture was heated in a microwave reactor at 90° C. for 1.75 h. The mixture was concentrated onto Celite® and was purified on an silica gel column (4 g), eluting with CH₂Cl₂ containing 0-1% MeOH and 0-0.1% NH₄OH. The crude product was triturated with hot MeOH (1 mL) to isolated the desired product as an yellow solid (30.5 mg, 43%). 1H NMR (500 MHz, DMSO-d₆) δ 8.00-7.96 (m, 1H), 7.95-7.89 (m, 1H), 7.71-7.67 (m, 1H), 7.64 (s, 1H), 7.61-7.55 (m, 2H), 7.00-6.96 (m, 2H), 6.78-6.69 (m, 2H), 3.43-3.41 (m, 2H), 3.22-3.17 (m, 4H), 2.97 (br t, J=6.5 Hz, 2H), 2.70-2.66 (m, 1H), 2.61-2.57 (m, 4H), 1.01 (d, J=6.5 Hz, 6H); LCMS: [M+H]⁺=477.63.

### Example 8: 6-(3-amino-5-chloro-6-(4-(4-isopropylpiperazin-1-yl)phenyl)pyrazin-2-yl)-7-fluoro-3,4-dihydroisoquinolin-1(2H)-one (I-8)

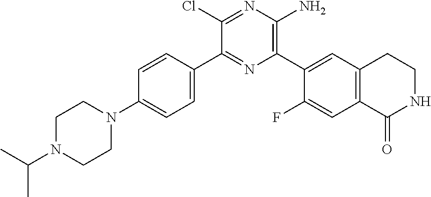 [SMILES: CC(C)N1CCN(c2ccc(-c3nc(-c4cc5c(cc4F)C(=O)NCC5)c(N)nc3Cl)cc2)CC1]

### Step 1: 8-fluoro-6-(4,4,5,5-tetramethyl-1,3,2-dioxaborolan-2-yl)-3,4-dihydroiso-quinolin-1(2H)-one

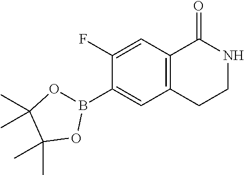 [SMILES: CC1(C)OB(c2cc3c(cc2F)C(=O)NCC3)OC1(C)C]

To 6-bromo-5-fluoro-3,4-dihydroisoquinolin-1(2H)-one (50 mg, 0.205 mmol, 1 eq), bis(pinacolato)diboron (58.2 mg, 0.229 mmol, 1.1 eq), KOAc (61.3 mg, 0.625 mmol, 3 eq) and [1,12-bis(diphenylphosphino)ferrocene]dichloropalladium(II) (7.62 mg, 10.41 μmol, 0.05 eq) in 1,4-dioxane (2.5 mL) was added under argon. The mixture was heated in a microwave for 2 h at 100° C. The reaction mixture was used in the next step as a solution without purification. LCMS: [M+H]⁺=292.0

### Step 2: 6-(3-amino-6-bromo-5-chloropyrazin-2-yl)-7-fluoro-3,4-dihydroisoquinolin-1(2H)-one

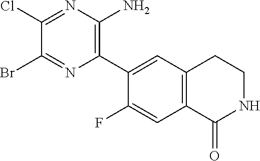 [SMILES: Nc1nc(Cl)c(Br)nc1-c1cc2c(cc1F)C(=O)NCC2]

Water (12 mL) was added to a degassed suspension of 5-bromo-6-chloro-3-iodopyrazin-2-amine (570 mg, 1.70 mmol), 7-fluoro-6-(4,4,5,5-tetramethyl-1,3,2-dioxaborolan-2-yl)-3,4-dihydroisoquinolin-1(2H)-one (500 mg, 1.49 mmol), Na₂CO₃H₂O (389 mg, 3.14 mmol) and bis(triphenylphosphine)palladium(II) dichloride (126 mg, 0.179 mmol) in CH₃CN (28 ml) and the reaction mixture was heated in a microwave reactor at 80° C. for 90 min. Additional 7-fluoro-6-(4,4,5,5-tetramethyl-1,3,2-dioxaborolan-2-yl)-3,4-dihydroisoquinolin-1(2H)-one (150 mg, 0.447 mmol) was added and the mixture was heated at 80° C. for an additional 2 h. The reaction was partitioned between water and EtOAc, the organic phase was separated, and the aqueous phase was extracted with 4:1 CHCl₃/i-PrOH (4×). The combined organic layers were dried over MgSO4, filtered and concentrated onto Celite®. Purification by flash silica gel chromatography (12 g column, eluting with CH₂Cl₂ containing 0-1.5% MeOH and 0-0.15% NH₄OH), and combining and concentrating the appropriate fractions, afforded the desired product as a beige solid (310 mg, 49%). LCMS [M+H]⁺=373.39.

### Step 3: 6-(3-amino-5-chloro-6-(4-(4-isopropylpiperazin-1-yl)phenyl)pyrazin-2-yl)-7-fluoro-3,4-dihydroisoquinolin-1(2H)-one (I-8)

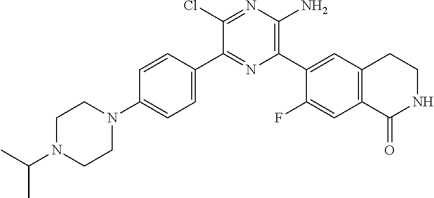 [SMILES: CC(C)N1CCN(c2ccc(-c3nc(-c4cc5c(cc4F)C(=O)NCC5)c(N)nc3Cl)cc2)CC1]

A microwave vial containing a mixture of 6-(3-amino-6-bromo-5-chloropyrazin-2-yl)-7-fluoro-3,4-dihydroisoquinolin-1(2H)-one (50 mg, 0.135 mmol), 4-(4-isopropylpiperazinyl)phenylboronic acid, pinacol ester (62.2 mg, 0.188 mmol), [1,12-bis(diphenylphosphino)ferrocene]dichloropalladium(II) (14.77 mg, 0.020 mmol) and Cs₂CO₃ (132 mg, 0.404 mmol) in DME (3.5 mL) was degassed with argon. Water (1 mL) was added and the mixture was heated in a microwave reactor at 90° C. for 1.75 h. The mixture was concentrated onto Celite® and was purified by flash silica gel chromatography (4 g column), eluting with CH₂Cl₂ containing 0-2% MeOH and 0-0.2% NH₄OH. The crude product that was isolated contained impurities. It was therefore triturated with hot MeOH (1 mL) to isolate the desired product as a yellow solid (33 mg, 47%). ¹H NMR (500 MHz, DMSO-d₆) δ 8.18-8.11 (m, 1H), 7.66-7.60 (m, 1H), 7.58-7.53 (m, 2H), 7.52-7.48 (m, 1H), 6.99-6.95 (m, 2H), 6.76-6.65 (m, 2H), 3.42-3.39 (m, 2H), 3.20-3.16 (m, 4H), 2.96-2.91 (m, 2H), 2.70-2.65 (m, 1H), 2.60-2.56 (m, 4H), 1.03-0.99 (m, 6H); LCMS: [M+H]⁺=495.62.

### Example 9: 6-(3-amino-5-fluoro-6-(4-(1-isopropyl-1,2,3,6-tetrahydropyridin-4-yl)phenyl)pyrazin-2-yl)-7-fluoro-3,4-dihydroisoquinolin-1(2H)-one (I-9)

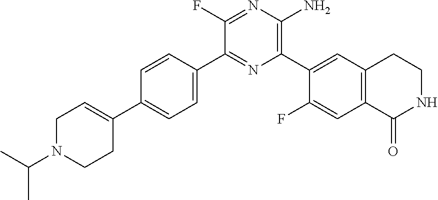 [SMILES: CC(C)N1CC=C(c2ccc(-c3nc(-c4cc5c(cc4F)C(=O)NCC5)c(N)nc3F)cc2)CC1]

### Step 1: tert-butyl 4-(4-bromophenyl)-3,6-dihydropyridine-1(2H)-carboxylate

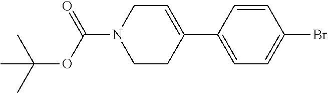 [SMILES: CC(C)(C)OC(=O)N1CC=C(c2ccc(Br)cc2)CC1]

Water (1.5 mL) was added to mixture of 1-bromo-4-iodobenzene (2.5 g, 8.84 mmol), Na₂CO₃H₂O (3.29 g, 26.5 mmol), tetrakis(triphenylphosphine)palladium(0) (0.511 g, 0.442 mmol) and tert-butyl-4-(4,4,5,5-tetramethyl-1,3,2-dioxaborolan-2-yl)-1,2,3,6-tetrahydropyridine-1-carboxylate (3.01 g, 9.72 mmol) in CH₃CN (3.5 mL) under argon. The mixture was heated in a MW reactor at 90° C. for 1 h. The mixture was allowed to cool to RT, filtered through a thin pad of Celite®, concentrated and purified on an Isco column (24 g), eluting with hexanes containing 0-5% EA. The appropriate fractions were combined and concentrated to yield the desired product as a brown oil (2.477 g, 83%). LCMS: [M+H]+=284.38.

### Step 2: 4-(4-bromophenyl)-1,2,3,6-tetrahydropyridine

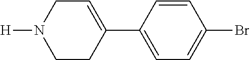 [SMILES: Brc1ccc(C2=CCNCC2)cc1]

Trifluoroacetic acid (1.250 ml, 16.32 mmol) was added to solution of tert-butyl 4-(4-bromophenyl)-3,6-dihydropyridine-1(2H)-carboxylate (1.38 g, 4.08 mmol) in CH₂Cl₂ (10 ml) under argon at RT. The reaction mixture was stirred at RT for 1 h. Complete conversion to the desired product was observed by LCMS. The mixture was concentrated and the residue was dissolved in MeOH (20 mL), passed through a 5 G porapak cation exchange cartridge and the eluant was removed to afford the product as a beige solid (854 mg, 88%). LCMS: [M+H]+=240.36.

### Step 3: 4-(4-bromophenyl)-1-isopropyl-1,2,3,6-tetrahydropyridine

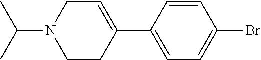 [SMILES: CC(C)N1CC=C(c2ccc(Br)cc2)CC1]

A solution of 4-(4-bromophenyl)-1,2,3,6-tetrahydropyridine (500 mg, 2.10 mmol), propan-2-one (0.311 ml, 4.20 mmol) and AcOH (0.060 mL, 1.05 mmol) in dichloroethane (15 mL) at RT was treated with NaBH(OAc)₃ (1335 mg, 6.30 mmol). The mixture was stirred at RT for 5 h. The mixture was concentrated to dryness and passed through a porapak cation exchange resin cartridge. The desired product was isolated as an off-white solid (575 mg, 98%) as an off-white solid. LCMS: [M+H]+=280.43.

### Step 4: 1-isopropyl-4-(4-(4,4,5,5-tetramethyl-1,3,2-dioxaborolan-2-yl)phenyl)-1,2,3,6-tetrahydropyridine

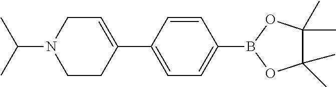 [SMILES: CC(C)N1CC=C(c2ccc(B3OC(C)(C)C(C)(C)O3)cc2)CC1]

A mixture of 4-(4-bromophenyl)-1-isopropyl-1,2,3,6-tetrahydropyridine (150 mg, 0.535 mmol), bis(pinacolato)diboron (154 mg, 0.605 mmol), [1,12-bis(diphenylphosphino)ferrocene]dichloropalladium(II) (39.2 mg, 0.054 mmol) and KOAc (158 mg, 1.61 mmol) in 1,4-dioxane (6 mL), was flushed with argon. The mixture was heated at 100° C. in a MW reactor for 1 h. The reaction mixture was filtered through a thin pad of Celite® and the filter cake was washed using EtOAc. The combined filtrate was concentrated to dryness and the residue was dissolved in DME to make 10 mL stock solution. The concentration is 17.5 mg/mL assuming 100% conversion. LCMS: [M+H]⁺=328.64.

### Step 6: 3-(4-bromo-3-fluorophenyl) propionic acid

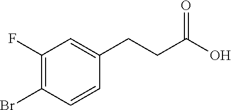 [SMILES: O=C(O)CCc1ccc(Br)c(F)c1]

To a stirred solution of triethylamine (123 mL, 886.6 mmol) was added formic acid (83.6 mL, 2216.7 mmol) portion wise and the mixture was stirred for 15 minutes at RT. The mixture was then diluted with DMF (750 mL) and 3-bromo-4-fluorobenzaldehyde (150 g, 738.9 mmol), Meldrum's acid (106.4 g, 738.9 mmol) was added. The mixture was then heated at 100° C. for 16 h. The reaction mixture was poured into ice cold water (3.5 It) and conc. HCl (350 mL). The mixture was extracted with DCM (2×1000 mL). The combined organic layers were washed with 1 N NaOH (2×1500 mL). The aqueous layer was acidified with conc. HCl and extracted with EtOAc (2×1000 mL). The combined organic layers were dried over Na₂SO₄ and concentrated under reduced pressure to afford the product (120 g, 65%) as brown liquid. This was used directly to next step. LCMS: [M+H]⁺=247.06.

### Step 7: 5-bromo-6-fluoro-2,3-dihydro-1H-inden-1-one

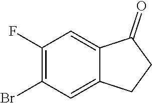 [SMILES: O=C1CCc2cc(Br)c(F)cc21]

To a stirred solution of 3-(4-bromo-3-fluorophenyl) propionic acid (120 g, 487.8 mmol) in DCM (1000 mL) was added oxalylchloride (146.5 mL, 1707.3 mmol) and DMF (16 mL) at RT for 30 minutes. The solvent was concentrated under reduced pressure to give crude residue. The residue was dissolved in DCM (1000 mL) and was added dropwise to a stirred solution aluminum chloride (227 g, 1707.3 mmol) in DCM (4000 mL). The mixture was stirred for 2 h at RT. The mixture was poured into ice cold water (4000 mL) and extracted with DCM (2×1000 mL). The organic layers were dried over Na₂SO₄ concentrated under reduced pressure. The residue was purified via column chromatography silica gel (230-400 mesh) as an eluent 0-10% EtOAc in pet ether to afford the product (60 g, 64%) as an off white solid. LCMS: [M+H]⁺=229.05.

### Step 8: Synthesis of 6-bromo-7-fluoro-3,4-dihydroisoquinolin-1(2H)-one

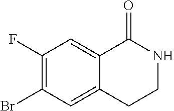 [SMILES: O=C1NCCc2cc(Br)c(F)cc21]

To a stirred solution of 5-bromo-6-fluoro-2,3-dihydro-1H-inden-1-one (60 g, 263.1 mmol) in DCM (900 mL) and methane sulfonic acid (240 mL, 4 V) was added NaN₃ (51.3 g, 789.4 mmol) portion wise at 0° C. for 4 h. The mixture was basified with 20% aqueous NaOH solution (600 mL) and extracted with DCM (2×800 mL). The combined organic layer was dried over Na₂SO₄ and concentrated under reduced pressure to give crude product which was purified by column chromatography silica gel (230-400 mesh) using 0-90% EtOAc in hexane as an eluent to afford the product (30 g, 46.9%) as an off white solid. LCMS: [M+H]⁺=244.06.

### Step 9: 7-fluoro-6-(4,4,5,5-tetramethyl-1,3,2-dioxaborolan-2-yl)-3,4-dihydroisoquinolin-1 (2H)-one

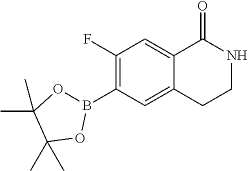 [SMILES: CC1(C)OB(c2cc3c(cc2F)C(=O)NCC3)OC1(C)C]

To a stirred solution of 6-bromo-7-fluoro-3,4-dihydroisoquinolin-1(2H)-one (30 g, 123.4 mmol) in 1,4-Dioxane (300 mL) was added KOAc (36.3 g, 370.3 mmol) bis(pinacolato)diboron (46.8 g, 185.1 mmol) at RT then de-gassed with argon for 30 minutes and followed by was added Pd(dppf)Cl₂·DCM(0) (10.1 g, 12.3 mmol) and the reaction mixture was heated at 90° C. for 16 h before cooling to RT. The reaction mixture was filtered through celite bed and washed with EtOAc (1000 mL) and the filtrate was concentrated under reduced pressure gave crude compound. The crude compound was washed with diethyl ether (300 mL), filtered and dried to afford the product (26 g, 72%) as a dark brown solid. TLC (100% EtOAc): R_{f}=0.4

### Step 10: 6-(3-amino-6-bromo-5-fluoropyrazin-2-yl)-7-fluoro-3,4-dihydroisoquinolin1(2H)-one

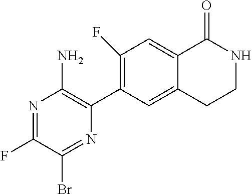 [SMILES: Nc1nc(F)c(Br)nc1-c1cc2c(cc1F)C(=O)NCC2]

To a stirred solution of 5-bromo-6-fluoro-3-iodopyrazin-2-amine (9 g, 28.4 mmol) in 1,4-sioxane (180 mL) was added 7-fluoro-6-(4,4,5,5-tetramethyl-1,3,2-dioxaborolan-2-yl)-3,4-dihydroisoquinolin-1 (2H)-one (12.4 g, 42.6 mmol), Na₂CO₃ (9 g, 85.1 mmol and 42 mL of water) at RT. The mixture was de-gassed with argon for 30 mins and followed by was added Pd(dppf)Cl₂·DCM (2.3 g, 2.8 mmol) and the reaction mixture was heated at 80° C. for 16h before cooling to RT. The reaction mixture was filtered through celite bed and washed ethyl acetate (2×500 mL). The filtrate was washed with cold water (1000 mL), separated the organic layer then dried over Na₂SO₄ and concentrated under reduced pressure gave crude compound. which was purified by column chromatography (Neutral alumina) by using 0-100% EtOAc in Pet ether and 0-5% MeOH in DCM as eluent to afford the product (4.5 g, 45%) as pale brown solid. TLC (5% MeOH in DCM) R_{f}=0.4

### Step 11: 6-(3-amino-5-fluoro-6-(4-(1-isopropyl-1,2,3,6-tetrahydropyridin-4-yl)phenyl)pyrazin-2-yl)-7-fluoro-3,4-dihydroisoquinolin-1(2H)-one

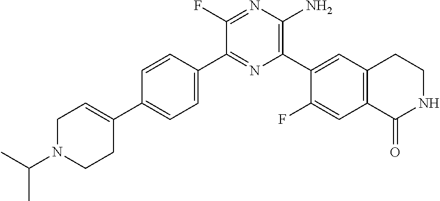 [SMILES: CC(C)N1CC=C(c2ccc(-c3nc(-c4cc5c(cc4F)C(=O)NCC5)c(N)nc3F)cc2)CC1]

A microwave vial containing a mixture of 6-(3-amino-6-bromo-5-fluoropyrazin-2-yl)-7-fluoro-3,4-dihydroisoquinolin-1(2H)-one (23 mg, 0.065 mmol), 1-isopropyl-4-(4-(4,4,5,5-tetramethyl-1,3,2-dioxaborolan-2-yl)phenyl)-1,2,3,6-tetrahydropyridine (1.58 mL, 0.084 mmol), [1,12-bis(diphenylphosphino)ferrocene]dichloropalladium(II) (7.11 mg, 9.71 μmol) and Cs₂CO₃ (63.3 mg, 0.194 mmol) in DME (4 mL) was degassed with argon. Water (1 mL) was added and the mixture was heated in a microwave reactor at 90° C. for 1.75 h. The mixture was concentrated onto Celite® and purified by silica gel chromatography (4 g column), eluting with CH₂Cl₂ containing 0-2.5% MeOH and 0-0.25% NH₄OH. The resulting crude product was subjected to further purification by preparative HPLC, eluting with water/CH₃CN/HCOOH (0.1%) yielded the desired product (8 mg, 26%) as a yellow solid. ¹H NMR (500 MHz, DMSO-d₆) δ 8.18-8.14 (m, 1H), 7.81 (br d, J=7.8 Hz, 2H), 7.65 (d, J=10.1 Hz, 1H), 7.57-7.50 (m, 3H), 6.99-6.77 (m, 2H), 6.32-6.19 (m, 1H), 3.54-3.52 (m, 4H), 3.19 (br d, J=2.6 Hz, 2H), 2.99-2.95 (m, 2H), 2.81-2.76 (m, 1H), 2.69 (br t, J=5.5 Hz, 2H), 1.07-1.02 (m, 6H); LCMS: [M+H]⁺=476.63.

### Example 10: 6-(3-amino-5-fluoro-6-(4-(1-isopropylpiperidin-4-yl)phenyl)pyrazin-2-yl)-8-fluoro-3,4-dihydroisoquinolin-1(2H)-one (I-10)

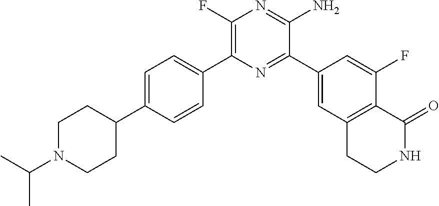 [SMILES: CC(C)N1CCC(c2ccc(-c3nc(-c4cc(F)c5c(c4)CCNC5=O)c(N)nc3F)cc2)CC1]

### Step 1: 8-fluoro-6-(4,4,5,5-tetramethyl-1,3,2-dioxaborolan-2-yl)-3,4-dihydroisoquinolin-1(2H)-one

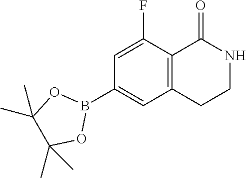 [SMILES: CC1(C)OB(c2cc(F)c3c(c2)CCNC3=O)OC1(C)C]

To a 100 mL round bottom flask charged with 6-bromo-8-fluoro-3,4-dihydroisoquinolin-1(2H)-one (896 mg, 3.67 mmol), bis(pinacolato)diboron (1025 mg, 4.04 mmol) and KOAc (1081 mg, 11.01 mmol), was added anhydrous 1,4-dioxane (40 mL). The system was flushed with nitrogen and PdCl₂dppf (269 mg, 0.367 mmol) was added. The mixture was further flushed with nitrogen then heated at 100° C. overnight. LCMS analysis showed a 58:42 mixture of the boronic acid and boronate. The reaction was diluted with acetonitrile, filtered through a pad of Celite®, and concentrated in vacuo. The crude material was used as-is in the next step assuming full conversion to the boronate. Boronate LCMS: [M+H]+=292.34, Boronic acid LCMS: [M+H]+=210.39.

### Step 2: 6-(3-amino-6-bromo-5-fluoropyrazin-2-yl)-8-fluoro-3,4-dihydroisoquinolin-1(2H)-one

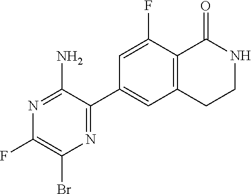 [SMILES: Nc1nc(F)c(Br)nc1-c1cc(F)c2c(c1)CCNC2=O]

To a solution of 5-bromo-6-fluoro-3-iodopyrazin-2-amine (760 mg, 2.39 mmol) and 8-fluoro-6-(4,4,5,5-tetramethyl-1,3,2-dioxaborolan-2-yl)-3,4-dihydroisoquinolin-1(2H)-one (835 mg, 2.87 mmol) in acetonitrile (15 mL), in a microwave vial, was added a solution of Na₂CO₃H₂O (623 mg, 5.02 mmol) in water (3.8 mL). The system was degassed and bis(triphenylphosphine)palladium(II) dichloride (201 mg, 0.287 mmol) was added. The reaction was heated in the microwave at 80° C. for 3 h. The reaction was concentrated onto Celite® and purified by silica gel chromatography, eluting with 0-100% EtOAc/hexanes, followed by silica gel chromatography eluting with 0-10% MeOH/CH₂Cl₂+1% NH₄OH to give the product (536 mg, 63%) as a beige solid. LCMS: [M+H]+=355.28.

### Step 3: tert-butyl 4-(4-(5-amino-3-fluoro-6-(8-fluoro-1-oxo-1,2,3,4-tetrahydroisoquinolin-6-yl)pyrazin-2-yl)phenyl)piperidine-1-carboxylate

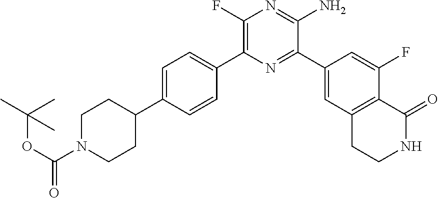 [SMILES: CC(C)(C)OC(=O)N1CCC(c2ccc(-c3nc(-c4cc(F)c5c(c4)CCNC5=O)c(N)nc3F)cc2)CC1]

To a degassed suspension of 6-(3-amino-6-bromo-5-fluoropyrazin-2-yl)-8-fluoro-3,4-dihydroisoquinolin-1(2H)-one (100 mg, 0.282 mmol), 4-(N-Boc-piperidino)phenylboronic acid pinacol ester (131 mg, 0.338 mmol) and Cs₂CO₃ (229 mg, 0.704 mmol) in a 3:1 mixture of DME (4 mL):water (1.3 mL) was added PdCl₂dppf (20.60 mg, 0.028 mmol). The mixture flushed with nitrogen and heated in the microwave at 90° C. for 2 hours. The reaction was concentrated onto Celite® and purified by silica gel chromatography, eluting with 0-10% MeOH/CH₂Cl₂+1% NH₄OH, to give the product (151 mg, 100%) as a brown solid. LCMS: [M+H]+=534.54.

### Step 4: 6-(3-amino-5-fluoro-6-(4-(piperidin-4-yl)phenyl)pyrazin-2-yl)-8-fluoro-3,4-dihydroisoquinolin-1(2H)-one

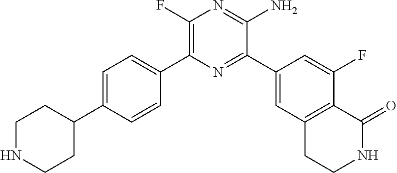 [SMILES: Nc1nc(F)c(-c2ccc(C3CCNCC3)cc2)nc1-c1cc(F)c2c(c1)CCNC2=O]

To a solution of tert-butyl 4-(4-(5-amino-3-fluoro-6-(8-fluoro-1-oxo-1,2,3,4-tetrahydroisoquinolin-6-yl)pyrazin-2-yl)phenyl)piperidine-1-carboxylate (151 mg, 0.282 mmol) in CH₂Cl₂ (5 mL) was added TFA (0.65 mL, 8.46 mmol). The reaction was stirred at RT overnight. The reaction was concentrated in vacuo to remove the volatiles. The crude was dissolved in methanol and passed through an Isolute SCX-2 cation exchange resin cartridge eluting with 3% NH₃/MeOH to give the product (120 mg, 98%) as a beige solid. LCMS: [M+H]+=436.58.

### Step 5: 6-(3-amino-5-fluoro-6-(4-(1-isopropylpiperidin-4-yl)phenyl)pyrazin-2-yl)-8-fluoro-3,4-dihydroisoquinolin-1(2H)-one

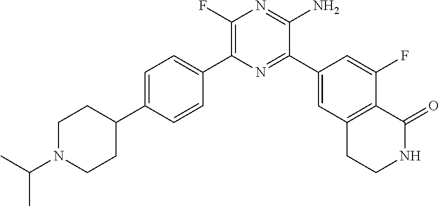 [SMILES: CC(C)N1CCC(c2ccc(-c3nc(-c4cc(F)c5c(c4)CCNC5=O)c(N)nc3F)cc2)CC1]

To a solution of 6-(3-amino-5-fluoro-6-(4-(piperidin-4-yl)phenyl)pyrazin-2-yl)-8-fluoro-3,4-dihydroisoquinolin-1(2H)-one (30 mg, 0.069 mmol) and N,N-diisopropylethylamine (0.120 mL, 0.689 mmol) in DMF (1 mL) was added 2-iodopropane (58.6 mg, 0.344 mmol). The reaction was stirred at RT for 2.5 d. The reaction was concentrated onto Celite® and purified by silica gel chromatography eluting with 0-10% MeOH/CH₂Cl₂+1% NH₄OH, followed by reverse phase chromatography (C18, 0-100% ACN/H₂O). The desired fractions were then passed through an Isolute SCX-2 cation exchange resin cartridge eluting with 3% NH₃ in MeOH to give the title compound (14.6 mg, 44%) as a yellow solid. ¹H NMR (500 MHz, DMSO-d₆) δ 8.05 (br s, 1H), 7.79 (br d, J=7.6 Hz, 2H), 7.50 (s, 1H), 7.44 (br d, J=11.9 Hz, 1H), 7.34 (d, J=8.2 Hz, 2H), 6.98 (s, 2H), 2.98 (br t, J=6.2 Hz, 2H), 2.90 (br d, J=11.1 Hz, 2H), 2.73 (td, J=6.5, 13.1 Hz, 1H), 2.24 (br t, J=10.8 Hz, 2H), 1.78 (br d, J=12.2 Hz, 2H), 1.68-1.59 (m, 2H), 1.24 (s, 1H), 1.00 (d, J=6.5 Hz, 6H); LCMS: [M+H]+=478.57.

### Example 11: 6-(3-amino-5-fluoro-6-(4-(4-isopropylpiperazin-1-yl)phenyl)pyrazin-2-yl)-3-((methylamino)methyl)-3,4-dihydroisoquinolin-1(2H)-one (I-11)

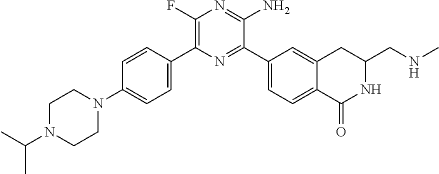 [SMILES: CNCC1Cc2cc(-c3nc(-c4ccc(N5CCN(C(C)C)CC5)cc4)c(F)nc3N)ccc2C(=O)N1]

### Step 1: 3-(3-bromophenyl)-1-methoxy-1-oxopropan-2-aminium chloride

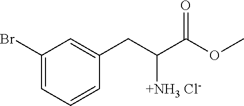

To a stirred solution of 2-(3-bromophenyl)-1-carboxyethan-1-aminium chloride (10 g, 35.7 mmol) in MeOH (300 mL) was added dropwise SOCl₂ (7.7 mL, 107.3 mmol) at 0° C. Then, the reaction mixture was heated at 100° C. for 16h before cooling to RT. The Solvent was concentrated under reduced pressure to afford the product (10.4 g, quant) as an off white solid. The crude compound was used in the next step without purification. LCMS: [M+H]⁺=258.09.

### Step 2: methyl 3-(3-bromophenyl)-2-(((trichloromethoxy)carbonyl)amino)propanoate

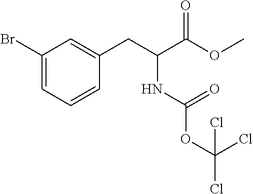 [SMILES: COC(=O)C(Cc1cccc(Br)c1)NC(=O)OC(Cl)(Cl)Cl]

To a stirred solution of 3-(3-bromophenyl)-1-methoxy-1-oxopropan-2-aminium chloride (10.5 g, 35.7 mmol) in 1, 4-dioxane (115 mL) was added trichloromethylchloroformate (5.9 mL, 50 mmol) at RT. Then, the mixture was heated at 100° C. for 16 h before cooling to RT. The solvent was concentrated under reduced pressure to afford the product (14.8 g, quant) as a pale brown gummy liquid which was used next step without purification. TLC: 10% MeOH: CH₂Cl₂; R_{f}=0.7.

### Step 3: methyl 6-bromo-1-oxo-1,2,3,4-tetrahydroisoquinoline-3-carboxylate

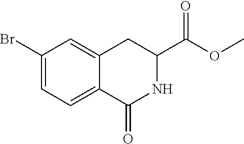 [SMILES: COC(=O)C1Cc2cc(Br)ccc2C(=O)N1]

To a stirred solution of methyl 3-(3-bromophenyl)-2-(((trichloromethoxy)carbonyl)amino)propanoate (14.8 g, 35.5 mmol, quant) in DCM (150 mL) was added AlCl₃ (9.3 g, 71.1 mmol) portionwise at 0° C. Then, the mixture was heated at 45° C. for 3 h before cooling to RT. The reaction mixture was quenched with water (100 mL) and extracted with CH₂Cl₂ (2×300 mL). The combined organic layers were dried over Na₂SO₄ and concentrated under reduced pressure to give crude residue. The crude compound was purified by column chromatography silica gel (100-200 mesh) using 0-60% EtOAc in Hexane as an eluent to afford the product (2.9 g, 29%) as an off-white solid. LCMS: [M+H]+=284.05.

### Step 4: 6-bromo-3-(hydroxymethyl)-3,4-dihydroisoquinolin-1(2H)-one

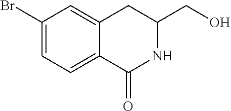 [SMILES: O=C1NC(CO)Cc2cc(Br)ccc21]

To a stirred solution of methyl 6-bromo-1-oxo-1,2,3,4-tetrahydroisoquinoline-3-carboxylate (2.9 g, 10.2 mmol) in EtOH (30 mL) was added portionwise LiBH₄ (450 mg, 20.4 mmol) at 0° C. The reaction was stirred at RT for 16h, and the solvent was concentrated under reduced pressure to give crude residue. The crude compound was diluted with water (100 mL) and extracted with CH₂Cl₂ (2×300 mL). The combined organic layers were dried over Na₂SO₄ and concentrated under reduced pressure to give crude residue. The crude compound was triturated with diethyl ether (100 mL), filtered and dried under vacuum to afford the product (2.5 g, 95%) as an off-white solid. LCMS: [M+H]⁺=256.03.

### Step 5: (6-bromo-1-oxo-1,2,3,4-tetrahydroisoquinolin-3-yl)methyl methanesulfonate

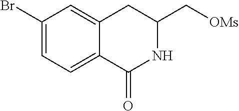 [SMILES: CS(=O)(=O)OCC1Cc2cc(Br)ccc2C(=O)N1]

To a stirred solution of 6-bromo-3-(hydroxymethyl)-3,4-dihydroisoquinolin-1(2H)-one (2.5 g, 9.8 mmol) in CH₂Cl₂ (30 mL) were added triethylamine (2.6 mL, 19.6 mmol), methanesulphonylchloride (1.5 mL, 19.6 mmol) and DMAP (110 mg, 0.98 mmol) at 0° C. Then, the reaction mixture was stirred at RT for 16 h. The solvent was concentrated under reduced pressure to give crude residue. The crude compound was diluted with water (100 mL) and extracted with CH₂Cl₂ (2×300 mL). The combined organic layers were dried over Na₂SO₄ and concentrated under reduced pressure to give crude residue. The crude compound was triturated with diethyl ether (100 mL) filtered and dried under vacuum to afford the product (2.2 g, 68%) as an off-white solid. LCMS: [M+H]⁺=334.03.

### Step 6: 6-bromo-3-((methylamino)methyl)-3,4-dihydroisoquinolin-1(2H)-one

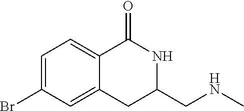

To a stirred solution of (6-bromo-1-oxo-1,2,3,4-tetrahydroisoquinolin-3-yl)methyl methanesulfonate (2.2 g, 6.6 mmol) in THF (25 mL) were added triethylamine (13.5 mL, 99 mmol) and 40% aqueous methylamine (13.5 mL) at RT in a sealed tube. The resulting mixture was stirred at 100° C. for 16 h before cooling to RT. The solvent was concentrated under reduced pressure to give crude residue. The crude compound was purified by column chromatography (neutral alumina) using 0-5% MeOH in CH₂Cl₂ as an eluent to afford the product (0.85 g, 48%) as an off white solid. ¹H NMR (400 MHz, DMSO-₆): δ 7.82 (s, 1H), 7.74 (d, J=8.4 Hz, 1H), 7.53 (dd, J=10.4, 2.4 Hz, 2H), 3.68-3.60 (m, 1H), 2.99 (dd, J=16, 4.8 Hz, 1H), 2.88-2.78 (m, 1H), 2.61-2.52 (m, 2H), 2.27 (s, 3H); LCMS: [M+H]⁺=269.04.

### Step 7: tert-butyl ((6-bromo-1-oxo-1,2,3,4-tetrahydroisoquinolin-3-yl)methyl)(methyl)carbamate

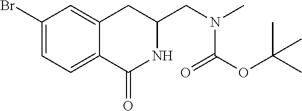 [SMILES: CN(CC1Cc2cc(Br)ccc2C(=O)N1)C(=O)OC(C)(C)C]

6-Bromo-3-((methylamino)methyl)-3,4-dihydroisoquinolin-1(2H)-one (200 mg, 0.743 mmol) was dissolved in CH₂Cl₂ (6 mL) at RT and di-tert-butyl dicarbonate (178 mg, 0.817 mmol) was added in one portion followed by N,N-diisopropylethylamine (0.272 mL, 1.56 mmol). The mixture was stirred at RT for 1 h, concentrated onto Celite® and purified by flash silica gel chromatography, eluting with hexanes containing 0-20% EA. The product was isolated as a colourless oil (359 mg, quantitative yield). LCMS: [M+H]⁺=371.45.

### Step 8: tert-butyl methyl((1-oxo-6-(4,4,5,5-tetramethyl-1,3,2-dioxaborolan-2-yl)-1,2,3,4-tetrahydroisoquinolin-3-yl)methyl)carbamate

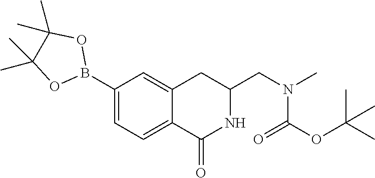 [SMILES: CN(CC1Cc2cc(B3OC(C)(C)C(C)(C)O3)ccc2C(=O)N1)C(=O)OC(C)(C)C]

A mixture of tert-butyl ((6-bromo-1-oxo-1,2,3,4-tetrahydroisoquinolin-3-yl)methyl)(methyl)carbamate (138. mg, 0.374 mmol), bis(pinacolato)diboron (107 mg, 0.422 mmol), [1,12-bis(diphenylphosphino)ferrocene]dichloropalladium(II) (27.3 mg, 0.037 mmol) and KOAc (110 mg, 1.12 mmol) in 1,4-dioxane (10 mL) was flushed with argon. The mixture was heated at 100° C. in a microwave reactor for 1 h. The mixture was filtered through a thin pad of Celite®, the filter cake was washed in with EtOAc, the combined filtrate was concentrated and a stock solution of 16 mg/mL was made in CH₃CN that was used in the next step. LCMS: [M+H]⁺=417.58.

### Step 9: tert-butyl methyl((1-oxo-6-(4,4,5,5-tetramethyl-1,3,2-dioxaborolan-2-yl)-1,2,3,4-tetrahydroisoquinolin-3-yl)methyl)carbamate

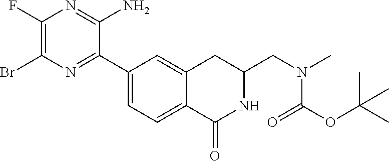 [SMILES: CN(CC1Cc2cc(-c3nc(Br)c(F)nc3N)ccc2C(=O)N1)C(=O)OC(C)(C)C]

Water (1.5 mL) was added to a degassed suspension of tert-butyl methyl((1-oxo-6-(4,4,5,5-tetramethyl-1,3,2-dioxaborolan-2-yl)-1,2,3,4-tetrahydroisoquinolin-3-yl)methyl)carbamate (6.3 mL of a solution with a concentration of 16 mg/mL, 100 mg, 0.239 mmol), 5-bromo-6-fluoro-3-iodopyrazin-2-amine (80 mg, 0.252 mmol), Na₂CO₃H₂O (65.5 mg, 0.528 mmol) and bis(triphenylphosphine)palladium(II) dichloride (21.20 mg, 0.030 mmol) and the reaction mixture flushed with argon. The mixture was heated in a microwave reactor at 80° C. for 1 h. The reaction mixture was concentrated onto Celite® and was purified by silica gel chromatography, eluting with hexanes containing 0-60% EtOAc. The product (80 mg, 60%) was isolated as an yellowish brown solid. LCMS: [M+H]⁺=480.39.

### Step 10: tert-butyl ((6-(3-amino-5-fluoro-6-(4-(4-isopropylpiperazin-1-yl)phenyl)pyrazin-2-yl)-1-oxo-1,2,3,4-tetrahydroisoquinolin-3-yl)methyl)(methyl)carbamate

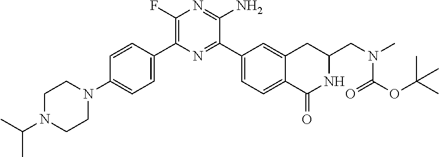 [SMILES: CC(C)N1CCN(c2ccc(-c3nc(-c4ccc5c(c4)CC(CN(C)C(=O)OC(C)(C)C)NC5=O)c(N)nc3F)cc2)CC1]

A microwave vial containing a mixture of tert-butyl ((6-(3-amino-6-bromo-5-fluoropyrazin-2-yl)-1-oxo-1,2,3,4-tetrahydroisoquinolin-3-yl)methyl)(methyl)carbamate (38 mg, 0.079 mmol), 4-(4-isopropylpiperazinyl)phenylboronic acid, pinacol ester (36.6 mg, 0.111 mmol), [1,12-bis(diphenylphosphino)ferrocene]dichloropalladium(II) (8.68 mg, 0.012 mmol) and Cs₂CO₃ (77 mg, 0.237 mmol) in DME (4 mL) was degassed with argon. Water (1 mL) was added and the mixture was heated in a microwave reactor at 90° C. for 1.75 h. The mixture was concentrated onto Celite® and was purified by flash silica gel chromatography, eluting with hexanes containing 0-100% EA. The product was isolated as an yellow solid (32 mg, 67%). LCMS: [M+H]+=604.73.

### Step 11: 6-(3-amino-5-fluoro-6-(4-(4-isopropylpiperazin-1-yl)phenyl)pyrazin-2-yl)-3-((methylamino)methyl)-3,4-dihydroisoquinolin-1(2H)-one

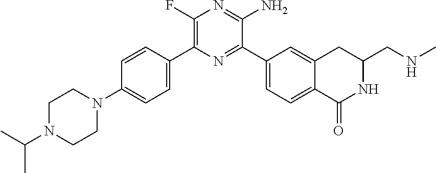 [SMILES: CNCC1Cc2cc(-c3nc(-c4ccc(N5CCN(C(C)C)CC5)cc4)c(F)nc3N)ccc2C(=O)N1]

Trifluoroacetic acid (0.107 mL, 1.39 mmol) was added to solution of tert-butyl ((6-(3-amino-5-fluoro-6-(4-(4-isopropylpiperazin-1-yl)phenyl)pyrazin-2-yl)-1-oxo-1,2,3,4-tetrahydroisoquinolin-3-yl)methyl)(methyl)carbamate (42 mg, 0.070 mmol) in CH₂Cl₂ (1.5 mL) at RT. The mixture was stirred at RT for 1 h and concentrated with a rotary evaporator. The residue was dissolved in MeOH, passed through a 2G porapak cation exchange cartridge to collect 26 mg of product. The crude product was triturated from CH₂Cl₂/hexanes to obtain the desired product as an yellow solid (16 mg, 43%). ¹H NMR (500 MHz, DMSO-d₆) δ 7.9-8.0 (m, 1H), 7.7-7.8 (m, 3H), 7.7-7.7 (m, 1H), 7.6-7.6 (m, 1H), 7.0-7.0 (m, 2H), 6.6-6.8 (m, 2H), 3.7-3.7 (m, 1H), 3.18 (br s, 4H), 3.0-3.1 (m, 1H), 2.9-2.9 (m, 1H), 2.6-2.7 (m, 3H), 2.58 (br s, 4H), 2.3-2.3 (m, 3H), 1.0-1.0 (m, 6H). LCMS: [M+H]⁺=504.58.

### Example 12: 6-(3-amino-5-fluoro-6-(4-morpholinophenyl)pyrazin-2-yl)-3-((methylamino)methyl)-3,4-dihydroisoquinolin-1(2H)-one (I-12)

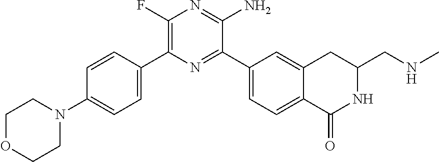 [SMILES: CNCC1Cc2cc(-c3nc(-c4ccc(N5CCOCC5)cc4)c(F)nc3N)ccc2C(=O)N1]

### Step 1: tert-butyl ((6-(3-amino-5-fluoro-6-(4-morpholinophenyl)pyrazin-2-yl)-1-oxo-1,2,3,4-tetrahydroisoquinolin-3-yl)methyl)(methyl)carbamate

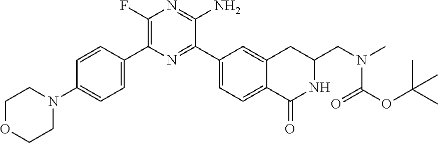 [SMILES: CN(CC1Cc2cc(-c3nc(-c4ccc(N5CCOCC5)cc4)c(F)nc3N)ccc2C(=O)N1)C(=O)OC(C)(C)C]

A microwave vial containing a mixture of tert-butyl ((6-(3-amino-6-bromo-5-fluoropyrazin-2-yl)-1-oxo-1,2,3,4-tetrahydroisoquinolin-3-yl)methyl)(methyl)carbamate (38 mg, 0.079 mmol), 4-(morpholino)phenylboronic acid (22.93 mg, 0.111 mmol), [1,12-bis(diphenylphosphino)ferrocene]dichloropalladium(II) (8.68 mg, 0.012 mmol) and Cs₂CO₃ (77 mg, 0.237 mmol) in DME (4 mL) was degassed with argon. Water (1 mL) was added and the mixture was heated in a microwave reactor at 90° C. for 1.5 h. The mixture was concentrated onto Celite® and was purified by flash silica gel chromatography eluting with hexanes containing 0-100% EtOAc. The product was isolated as an yellow solid (31 mg, 70%). LCMS: [M+H]⁺=563.62.

### Step 2: 6-(3-amino-5-fluoro-6-(4-morpholinophenyl)pyrazin-2-yl)-3-((methylamino)methyl)-3,4-dihydroisoquinolin-1(2H)-one

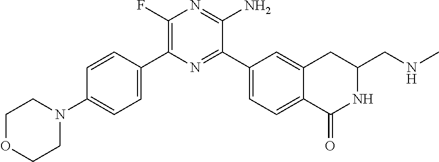 [SMILES: CNCC1Cc2cc(-c3nc(-c4ccc(N5CCOCC5)cc4)c(F)nc3N)ccc2C(=O)N1]

Trifluoroacetic acid (0.084 ml, 1.10 mmol) was added to solution of tert-butyl ((6-(3-amino-5-fluoro-6-(4-morpholinophenyl)pyrazin-2-yl)-1-oxo-1,2,3,4-tetrahydroisoquinolin-3-yl)methyl)(methyl)carbamate (31 mg, 0.055 mmol) in CH₂Cl₂ (1.5 mL) at RT. The mixture was stirred at RT for 1 h and the residue was dissolved in MeOH, passed through a 2 g porapak cation exchange cartridge to collect the desired product as an yellow solid (23 mg, 86%). ¹H NMR (500 MHz, DMSO-d₆) δ 7.99-7.91 (m, 1H), 7.84-7.73 (m, 3H), 7.72-7.67 (m, 1H), 7.64 (s, 1H), 7.08-6.99 (m, 2H), 6.81-6.64 (m, 2H), 3.79-3.71 (m, 5H), 3.20-3.15 (m, 4H), 3.10-3.05 (m, 1H), 2.93-2.86 (m, 1H), 2.64 (br s, 2H), 2.41-2.27 (m, 3H). LCMS: [M+H]⁺=463.53.

### Example 13: 6-(3-amino-6-(4-(1-(cyclopropylmethyl)piperidin-4-yl)phenyl)-5-fluoropyrazin-2-yl)-8-fluoro-3,4-dihydroisoquinolin-1(2H)-one Formate (I-13)

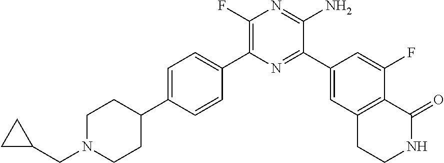 [SMILES: Nc1nc(F)c(-c2ccc(C3CCN(CC4CC4)CC3)cc2)nc1-c1cc(F)c2c(c1)CCNC2=O]

### Step 1: 5-bromo-6-fluoropyrazin-2-amine

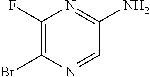

To an ice-cold solution of 6-fluoropyrazin-2-amine (550 mg, 4.86 mmol) in CH₂Cl₂ (30 mL) and CH₃CN (5.0 mL). A cold solution of recrystallized N-bromosuccinimide (883 mg, 4.96 mmol) in CH₂Cl₂ (10 mL) in CH₃CN (5.00 mL) was added dropwise over 10 min. After stirring at 0° C. for 5 min the reaction was quenched with ice/MeOH and concentrated onto Celite®. The crude material was purified by flash silica gel chromatography with 0-40% EtOAc/hexanes. The desired fractions were collected, concentrated and dried under high vacuum at RT to afford the product as a pale yellow solid. ¹H NMR (500 MHz, DMSO-d₆) δ 7.62 (d, J=4.4 Hz, 1H), 7.13 (br s, 2H).

### Step 2: 5-bromo-6-fluoro-3-iodopyrazin-2-amine

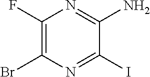

To a solution of 5-bromo-6-fluoropyrazin-2-amine (934 mg, 4.86 mmol) in AcOH (10 ml) was added N-iodosuccinimide (1313 mg, 5.84 mmol) portionwise. Trifluoroacetic acid (55.5 mg, 0.486 mmol) was added then the reaction mixture was heated at 50° C. for 1 hour. The reaction mixture was concentrated in vacuo and neutralized with aq NaHCO₃ (aq) and saturated aqueous sodium thiosulfate. The aqueous layer was extracted with CH₂Cl₂. The combined organic extracts were concentrated onto Celite® and purified using flash silica gel chromatography eluting with 0-30% EtOAc/hexanes. The desired fractions were collected, concentrated and dried under vacuum to afford the product (2.85 mmol, 59%) as a pale yellow solid. LCMS: [M+H]⁺=318.29.

### Step 3: 6-(3-amino-6-bromo-5-fluoropyrazin-2-yl)-8-fluoro-3,4-dihydroisoquinolin-1(2H)-one

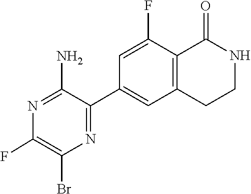 [SMILES: Nc1nc(F)c(Br)nc1-c1cc(F)c2c(c1)CCNC2=O]

2.391 mmol) and 8-fluoro-6-(4,4,5,5-tetramethyl-1,3,2-dioxaborolan-2-yl)-3,4-dihydroisoquinolin-1(2H)-one (835 mg, 2.87 mmol) in CH₃CN (15 mL), in a microwave vial, was added a solution of Na₂CO₃ (623 mg, 5.02 mmol) in water. The vessel was degassed and bis(triphenylphosphine)palladium(II) dichloride (201 mg, 0.287 mmol) was added. The reaction was heated in the microwave at 80° C. for 3 h. The reaction was concentrated onto Celite® and purified using flash silica gel chromatography, eluting with 0-100% EtOAc/hexanes. The desired fractions were collected, concentrated and re-purified using silica gel chromatography eluting with 0-10% MeOH/CH₂Cl₂+1% NH₄OH. The desired fractions were collected, concentrated and dried under high vacuum to afford the product (1.51 mmol, 63%) as a beige solid (over 2 steps). LCMS: [M+H]⁺=355.28.

### Step 4: tert-butyl 4-(4-(5-amino-3-fluoro-6-(8-fluoro-1-oxo-1,2,3,4-tetrahydroisoquinolin-6-yl)pyrazin-2-yl)phenyl)piperidine-1-carboxylate

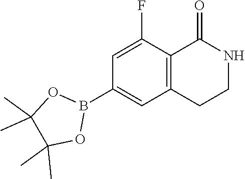 [SMILES: CC1(C)OB(c2cc(F)c3c(c2)CCNC3=O)OC1(C)C]

To a microwave vial containing 6-bromo-8-fluoro-3,4-dihydroisoquinolin-1(2H)-one (700 mg, 2.87 mmol), bis(pinacolato)diboron (801 mg, 3.15 mmol) and KOAc (844 mg, 8.60 mmol) was added anhydrous 1,4-dioxane (40.0 ml). The reaction was degassed and [1,12-bis(diphenylphosphino)ferrocene]dichloropalladium(II) (210 mg, 0.287 mmol) was added. The mixture was degassed then heated at 100° C. for 3 h. The reaction was diluted with CH₃CN, filtered through a pad of Celite®, concentrated in vacuo and used in the next step without further purification. LCMS: [M+H]⁺=292.40.

### Step 5: tert-butyl 4-(4-(5-amino-3-fluoro-6-(8-fluoro-1-oxo-1,2,3,4-tetrahydroisoquinolin-6-yl)pyrazin-2-yl)phenyl)piperidine-1-carboxylate

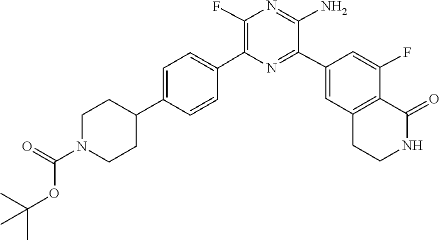 [SMILES: CC(C)(C)OC(=O)N1CCC(c2ccc(-c3nc(-c4cc(F)c5c(c4)CCNC5=O)c(N)nc3F)cc2)CC1]

To a degassed suspension of 6-(3-amino-6-bromo-5-fluoropyrazin-2-yl)-8-fluoro-3,4-dihydroisoquinolin-1(2H)-one (100 mg, 0.282 mmol), and Cs₂CO₃ (229 mg, 0.704 mmol) in a mixture of 1,2-DME (4 mL):H₂O (1.33 mL) was added 4-(N-Boc-piperidino)phenylboronic acid pinacol ester (131 mg, 0.338 mmol). The mixture was heated in the microwave at 90° C. for 2 h. The reaction was concentrated onto Celite® and purified by flash silica gel chromatography eluting with 0-10% MeOH/DCM+1% NH₄OH. The desired fractions were collected, concentrated and dried en vacuo to afford the product (0.282 mmol, 100%) as a brown solid. LCMS: [M+H]⁺=480.58.

### Step 6: 6-(3-amino-5-fluoro-6-(4-(piperidin-4-yl)phenyl)pyrazin-2-yl)-8-fluoro-3,4-dihydroisoquinolin-1(2H)-one

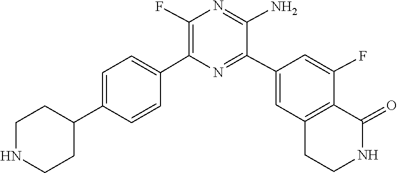 [SMILES: Nc1nc(F)c(-c2ccc(C3CCNCC3)cc2)nc1-c1cc(F)c2c(c1)CCNC2=O]

To a solution of tert-butyl 4-(4-(5-amino-3-fluoro-6-(8-fluoro-1-oxo-1,2,3,4-tetrahydroisoquinolin-6-yl)pyrazin-2-yl)phenyl)piperidine-1-carboxylate (151 mg, 0.282 mmol) in CH₂Cl₂ (5.0 mL) was added TFA (0.648 mL, 8.46 mmol). The reaction was stirred at RT for 16 h. The reaction was concentrated en vacuo to remove the volatiles. The crude product was dissolved in methanol and passed through an Isolute Flash SCX-2 500 mg exchange column eluting with 3% NH₃/MeOH, then concentrated and dried under high vacuum to afford the product (0.276 mmol, 98%). LCMS [M+H]⁺=436.58.

### Step 7: 6-(3-amino-6-(4-(1-(cyclopropylmethyl)piperidin-4-yl)phenyl)-5-fluoropyrazin-2-yl)-8-fluoro-3,4-dihydroisoquinolin-1(2H)-one formate

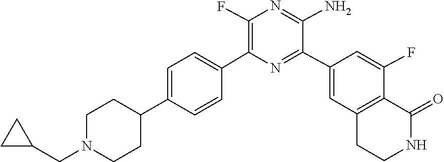 [SMILES: Nc1nc(F)c(-c2ccc(C3CCN(CC4CC4)CC3)cc2)nc1-c1cc(F)c2c(c1)CCNC2=O]

To a solution of 6-(3-amino-5-fluoro-6-(4-(piperidin-4-yl)phenyl)pyrazin-2-yl)-8-fluoro-3,4-dihydroisoquinolin-1(2H)-one (30 mg, 0.069 mmol) and N,N-diisopropylethylamine (0.120 mL, 0.689 mmol) in DMF (1 mL) was added (bromomethyl)cyclopropane (46.5 mg, 0.344 mmol). The reaction was stirred at RT for 72 h. The mixture was concentrated en vacuo and purified by Waters prep HPLC (XBridge Prep C18 5 μm, 10×100 mm Column, 90-70% 0.1% formic acid in water/0.1% formic acid in CH₃CN) to afford the formate salt of the title compound (18.8 mg, 51%) as a yellow solid. ¹H NMR (500 MHz, DMSO-d₆) δ 8.25-8.12 (m, 2H), 7.95 (br s, 1H), 7.69 (br d, J=7.5 Hz, 2H), 7.40 (s, 1H), 7.33 (br d, J=12.0 Hz, 1H), 7.25 (d, J=8.3 Hz, 2H), 6.88 (s, 2H), 3.26 (br d, J=2.8 Hz, 2H), 3.01 (br d, J=11.2 Hz, 2H), 2.87 (br t, J=6.2 Hz, 2H), 2.14 (d, J=6.5 Hz, 2H), 1.96 (br t, J=10.6 Hz, 2H), 1.70-1.55 (m, 4H), 0.82-0.71 (m, 1H), 0.44-0.34 (m, 2H), 0.00 (br d, J=4.3 Hz, 2H); LCMS: [M+H]+=490.54.

### Example 14: 6-(3-amino-5-fluoro-6-(4-(4-isopropylpiperazin-1-yl)phenyl)pyrazin-2-yl)-8-fluoro-3,4-dihydroisoquinolin-1(2H)-one (I-14)

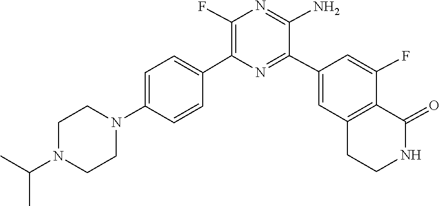 [SMILES: CC(C)N1CCN(c2ccc(-c3nc(-c4cc(F)c5c(c4)CCNC5=O)c(N)nc3F)cc2)CC1]

To a suspension of 6-(3-amino-6-bromo-5-fluoropyrazin-2-yl)-8-fluoro-3,4-dihydroisoquinolin-1(2H)-one (40 mg, 0.113 mmol), 4-(4-isopropylpiperazinyl)phenylboronic acid, pinacol ester (44.6 mg, 0.135 mmol) and Cs₂CO₃ (92 mg, 0.282 mmol) in a 3:1 mixture of DME (4 mL):water (1.3 mL) was added PdCl₂dppf (8.24 mg, 0.011 mmol). The mixture was flushed with nitrogen and heated in a microwave apparatus at 90° C. for 2 h. The reaction was concentrated onto Celite® and purified by silica gel chromatography eluting with 0-10% MeOH/CH₂Cl₂+1% NH₄OH. The combined concentrated fractions were triturated from MeOH to give the title compound (43.8 mg, 81%) as a yellow solid. ¹H NMR (500 MHz, DMSO-d₆) δ 8.04 (br s, 1H), 7.75 (d, J=7.9 Hz, 2H), 7.50 (s, 1H), 7.43 (d, J=12.1 Hz, 1H), 7.02 (d, J=8.9 Hz, 2H), 6.83 (s, 2H), 3.38-3.35 (m, 2H), 3.22-3.17 (m, 4H), 2.98 (br t, J=6.3 Hz, 2H), 2.68 (td, J=13.1, 6.5 Hz, 1H), 2.61-2.56 (m, 4H), 1.01 (d, J=6.6 Hz, 6H); LCMS: [M+H]+=479.57.

### Example 15: 6-(3-amino-5-fluoro-6-(4-((1R,5S)-3-isopropyl-3-azabicyclo[3.1.0]hexan-1-yl)phenyl)pyrazin-2-yl)-8-fluoro-3,4-dihydroisoquinolin-1(2H)-one formate

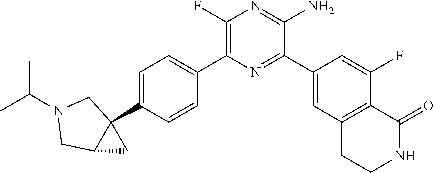 [SMILES: CC(C)N1C[C@H]2C[C@@]2(c2ccc(-c3nc(-c4cc(F)c5c(c4)CCNC5=O)c(N)nc3F)cc2)C1]

### Step 1: (1R,5S)-1-(4-(4,4,5,5-tetramethyl-1,3,2-dioxaborolan-2-yl)phenyl)-3-azabicyclo[3.1.0]hexane

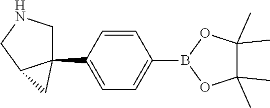 [SMILES: CC1(C)OB(c2ccc([C@]34CNC[C@H]3C4)cc2)OC1(C)C]

To a microwave vial charged with (1R,5S)-1-(4-bromophenyl)-3-azabicyclo[3.1.0]hexane (130 mg, 0.546 mmol), bis(pinacolato)diboron (152 mg, 0.601 mmol), KOAc (161 mg, 1.64 mmol), was added anhydrous 1,4-dioxane (6 mL). The system was degassed and PdCl₂dppf (39.9 mg, 0.055 mmol) was added. The mixture was flushed with nitrogen then heated at 100° C. 3 h. The reaction was diluted with CH₃CN, filtered through a pad of Celite®, concentrated in vacuo and used without further purification in the next step. LCMS: [M+H]+=286.51.

### Step 2: 6-(6-(4-((1R,5S)-3-azabicyclo[3.1.0]hexan-1-yl)phenyl)-3-amino-5-fluoropyrazin-2-yl)-8-fluoro-3,4-dihydroisoquinolin-1(2H)-one

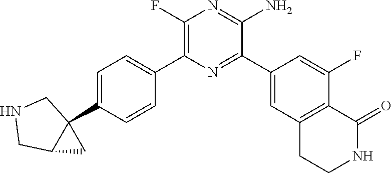 [SMILES: Nc1nc(F)c(-c2ccc([C@]34CNC[C@H]3C4)cc2)nc1-c1cc(F)c2c(c1)CCNC2=O]

To a suspension of 6-(3-amino-6-bromo-5-fluoropyrazin-2-yl)-8-fluoro-3,4-dihydroisoquinolin-1(2H)-one (95 mg, 0.267 mmol), (1R,5S)-1-(4-(4,4,5,5-tetramethyl-1,3,2-dioxaborolan-2-yl)phenyl)-3-azabicyclo[3.1.0]hexane (3.52 ml, 0.321 mmol, 26 mg/ml in CH₃CN) and Cs₂CO₃ (218 mg, 0.669 mmol) in a 3:1 mixture of DME (5 mL):water (1.7 mL) was added PdCl₂dppf (19.57 mg, 0.027 mmol). The mixture was flushed with nitrogen and heated in the microwave at 90° C. for 2 hours. The reaction was concentrated onto Celite® and purified by silica gel chromatography eluting with 0-10% MeOH/CH₂Cl₂+1% NH₄OH to afford the product (96 mg, 83%) as a brown solid. LCMS: [M+H]+=434.57.

### Step 3: 6-(3-amino-5-fluoro-6-(4-((1R,5S)-3-isopropyl-3-azabicyclo[3.1.0]hexan-1-yl)phenyl)pyrazin-2-yl)-8-fluoro-3,4-dihydroisoquinolin-1(2H)-one

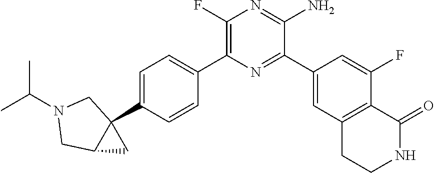 [SMILES: CC(C)N1C[C@H]2C[C@@]2(c2ccc(-c3nc(-c4cc(F)c5c(c4)CCNC5=O)c(N)nc3F)cc2)C1]

To a solution of 6-(6-(4-((1R,5S)-3-azabicyclo[3.1.0]hexan-1-yl)phenyl)-3-amino-5-fluoropyrazin-2-yl)-8-fluoro-3,4-dihydroisoquinolin-1(2H)-one (30 mg, 0.069 mmol) and N,N-diisopropylethylamine (0.121 mL, 0.692 mmol) in DMF (1 mL) was added 2-iodopropane (58.8 mg, 0.346 mmol). The reaction was stirred at RT for 3 d, then concentrated onto Celite® and purified by silica gel chromatography eluting with 0-10% MeOH/CH₂Cl₂+1% NH₄OH, followed by reverse phase chromatography (C18, 0-100% ACN/H₂O). The desired fractions were then passed through an Isolute SCX-2 cation exchange resin cartridge eluting with 3% NH₃ in MeOH. The material was then purified by Waters prep HPLC (XBridge Prep C18 5 μm, 10×100 mm Column, 90-70% 0.1% formic acid in water/0.1% formic acid in acetonitrile) to afford the formate salt of the title compound (11 mg, 31%) as an orange solid. ¹H NMR (500 MHz, DMSO-d₆) δ 8.32 (br s, 2H), 8.05 (br s, 1H), 7.78 (d, J=7.5 Hz, 2H), 7.50 (s, 1H), 7.43 (br d, J=11.9 Hz, 1H), 7.25 (d, J=8.3 Hz, 2H), 6.98 (s, 2H), 3.06 (br d, J=8.6 Hz, 1H), 2.98 (br t, J=6.2 Hz, 2H), 2.60 (d, J=8.4 Hz, 1H), 2.48-2.45 (m, 2H), 1.84 (td, J=7.8, 3.9 Hz, 1H), 1.33 (t, J=3.9 Hz, 1H), 1.05 (d, J=6.2 Hz, 3H), 1.02 (d, J=6.2 Hz, 3H), 0.77 (dd, J=3.5, 7.9 Hz, 1H); LCMS: [M+H]+=476.56

### Example 16: 6-(3-amino-6-(4-(4-(cyclopropylmethyl)piperazin-1-yl)phenyl)-5-fluoropyrazin-2-yl)-8-fluoro-3,4-dihydroisoquinolin-1(2H)-one (I-16)

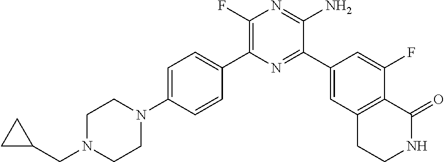 [SMILES: Nc1nc(F)c(-c2ccc(N3CCN(CC4CC4)CC3)cc2)nc1-c1cc(F)c2c(c1)CCNC2=O]

### Step 1: tert-butyl 4-(4-(5-amino-3-fluoro-6-(8-fluoro-1-oxo-1,2,3,4-tetrahydroisoquinolin-6-yl)pyrazin-2-yl)phenyl)piperazine-1-carboxylate

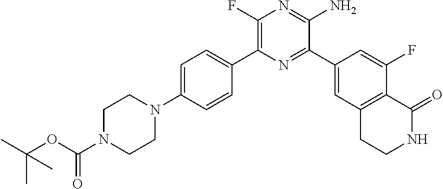 [SMILES: CC(C)(C)OC(=O)N1CCN(c2ccc(-c3nc(-c4cc(F)c5c(c4)CCNC5=O)c(N)nc3F)cc2)CC1]

To a suspension of 6-(3-amino-6-bromo-5-fluoropyrazin-2-yl)-8-fluoro-3,4-dihydroisoquinolin-1(2H)-one (70 mg, 0.197 mmol), tert-butyl 4-[4-(4,4,5,5-tetramethyl-1,3,2-dioxaborolan-2-yl)phenyl]tetrahydro-1(2H)-pyrazinecarboxylate (92 mg, 0.237 mmol) and Cs₂CO₃ (161 mg, 0.493 mmol) in a 3:1 mixture of DME (4 mL):water (1.3 mL) was added PdCl₂dppf (14.42 mg, 0.020 mmol). The mixture was heated in the microwave at 90° C. for 2 h. The reaction was concentrated onto Celite® and purified by silica gel chromatography eluting with 0-10% MeOH/CH₂Cl₂+1% NH₄OH to afford the product as a yellow solid. The material was carried directly onto the next step. LCMS: [M+H]⁺=537.55.

### Step 2: 6-(3-amino-5-fluoro-6-(4-(piperazin-1-yl)phenyl)pyrazin-2-yl)-8-fluoro-3,4-dihydroisoquinolin-1(2H)-one

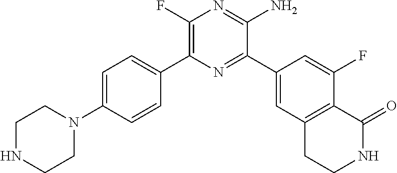 [SMILES: Nc1nc(F)c(-c2ccc(N3CCNCC3)cc2)nc1-c1cc(F)c2c(c1)CCNC2=O]

To a solution of tert-butyl 4-(4-(5-amino-3-fluoro-6-(8-fluoro-1-oxo-1,2,3,4-tetrahydroisoquinolin-6-yl)pyrazin-2-yl)phenyl)piperazine-1-carboxylate (106 mg, 0.197 mmol) in CH₂Cl₂ (4 mL) was added trifluoroacetic acid (0.45 ml, 5.91 mmol). The reaction was stirred at RT overnight. The reaction was concentrated in vacuo to remove the volatiles. The crude was dissolved in MeOH and passed through an Isolute SCX-2 cation exchange resin cartridge eluting with 3% NH₃ in MeOH to give the product (84 mg, 98%) as a bright yellow solid. LCMS: [M+H]⁺=437.45.

### Step 3: 6-(3-amino-6-(4-(4-(cyclopropylmethyl)piperazin-1-yl)phenyl)-5-fluoropyrazin-2-yl)-8-fluoro-3,4-dihydroisoquinolin-1(2H)-one

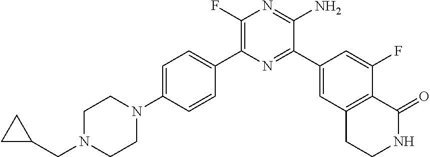 [SMILES: Nc1nc(F)c(-c2ccc(N3CCN(CC4CC4)CC3)cc2)nc1-c1cc(F)c2c(c1)CCNC2=O]

A procedure similar to Example 15, Step 3 using 6-(3-amino-5-fluoro-6-(4-(piperazin-1-yl)phenyl)pyrazin-2-yl)-8-fluoro-3,4-dihydroisoquinolin-1(2H)-one (30 mg, 0.069 mmol) and N,N-diisopropylethylamine (0.120 ml, 0.687 mmol) in DMF (1 mL) and (bromomethyl)cyclopropane (27.8 mg, 0.206 mmol) afforded the title compound (3.46 mg, 10%) as a yellow solid. ¹H NMR (500 MHz, DMSO-d₆) δ 8.03 (br s, 1H), 7.75 (br d, J=8.2 Hz, 2H), 7.50 (s, 1H), 7.42 (br d, J=12.2 Hz, 1H), 7.02 (br d, J=8.8 Hz, 2H), 6.83 (s, 2H), 3.22 (br s, 4H), 2.97 (br t, J=6.1 Hz, 2H), 2.61 (br s, 4H), 2.26 (br s, 2H), 0.87 (br d, J=6.4 Hz, 1H), 0.49 (br d, J=7.3 Hz, 2H), 0.11 (br d, J=3.7 Hz, 2H); LCMS: [M+H]+=491.54.

### Example 17: 6-(3-amino-5-fluoro-6-(4-morpholinophenyl)pyrazin-2-yl)-3,4-dihydroisoquinolin-1(2H)-one (I-17)

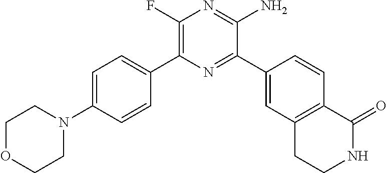 [SMILES: Nc1nc(F)c(-c2ccc(N3CCOCC3)cc2)nc1-c1ccc2c(c1)CCNC2=O]

To a degassed suspension of 6-(3-amino-6-chloro-5-fluoropyrazin-2-yl)-3,4-dihydroisoquinolin-1(2H)-one (80 mg, 0.273 mmol), 4-(morpholino)phenylboronic acid (85 mg, 0.410 mmol) and K₃PO₄ (145 mg, 0.683 mmol) in a 8:1 mixture of 1,4-dioxane (9.0 mL):water (1.1 mL) was added XPhos Pd G2 (32.3 mg, 0.041 mmol). The system was degassed and the mixture was heated at 90° C. for 16 h. The mixture was concentrated onto Celite® and purified by flash chromatography (silica gel) eluting with 0-10% MeOH/CH₂Cl₂+1% NH₄OH. The desired fractions were collected and concentrated. The yellow solid was triturated from MeOH:EtOAc (9:1, 15 mL) and dried under vacuum to afford the title compound (46.4 mg, 41%) as a yellow solid. ¹H NMR (500 MHz, DMSO-d₆) δ 7.98 (br. s., 1H), 7.94 (d, J=7.7 Hz, 1H), 7.77 (d, J=8.0 Hz, 2H), 7.69 (d, J=7.8 Hz, 1H), 7.65 (s, 1H), 7.03 (d, J=7.7 Hz, 2H), 6.74 (br. s., 2H), 3.75 (br. s., 4H), 3.42 (br. s., 2H), 3.17 (br. s., 4H), 2.98 (br. s., 2H); LCMS: [M+H]⁺=420.55.

### Example 18 (S)-6-(3-amino-5-fluoro-6-(3-(pyrrolidin-2-yl)-4-(tetrahydro-2H-pyran-4-yl)phenyl)pyrazin-2-yl)-3,4-dihydroisoquinolin-1(2H)-one (I-18)

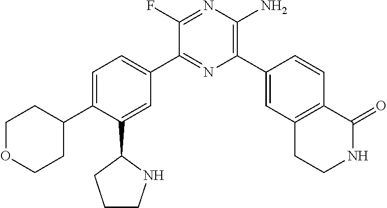 [SMILES: Nc1nc(F)c(-c2ccc(C3CCOCC3)c([C@@H]3CCCN3)c2)nc1-c1ccc2c(c1)CCNC2=O]

### Step 1: 1-(4-bromophenyl)-3-chloropropan-1-one

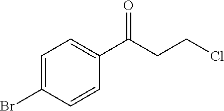 [SMILES: O=C(CCCl)c1ccc(Br)cc1]

To a stirred solution of AlCl₃ (699 g, 5254.8 mmol) in DCM (2.5 L), was added bromobenzene (500 mL, 4777.1 mmol) and 3-chloropropanoyl chloride (456 mL, 4777.1 mmol) in DCM (2.5 L) dropwise at 0° C. The reaction mixture was stirred at RT for 16 h. The mixture was quenched with ice water (5 L) and extracted with DCM (3×3.0 L). The combined organic layers were dried over Na₂SO₄ and concentrated under reduced pressure to give the product (700 g, quant) as a pale-yellow semi solid. TLC: (20% EA in Pet ether) R_{f}=0.4.

### Step 2: 5-bromo-2, 3-dihydro-1H-inden-1-one

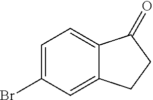 [SMILES: O=C1CCc2cc(Br)ccc21]

A solution of 1-(4-bromophenyl)-3-chloropropan-1-one (350 g, 1422.8 mmol) in H₂SO₄ (3.5 L) at was stirred at RT and heated to 100° C. for and stirred for 4 h before cooling to RT. The reaction mixture was diluted with ice water and extracted with EtOAc (3×5.0 L). The combined organic layer was washed with brine solution (4.0 L), dried over with Na₂SO₄, filtered and concentrated under reduced pressure to afford the product (500 g, quant) as a brown solid. LCMS: [M+H]+=211.06

### Step 3: 6-bromo-3,4-dihydroisoquinolin-1(2H)-one

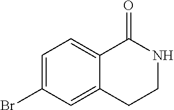 [SMILES: O=C1NCCc2cc(Br)ccc21]

To a stirred solution of 5-bromo-2, 3-dihydro-1H-inden-1-one (500 g, 2369.7 mmol) in DCM (4.0 L) was added methane sulfonic acid (2.0 L) at 0° C. Then added NaN₃ (539 g, 8.293 mmol) by portion wise. The reaction mixture was stirred for 30 min at RT. The reaction mixture was slightly basified with 20% NaOH solution and extracted with DCM (2×5.0 L), the combined organic layers was dried over sodium sulphate and concentrated to give crude compound; which was purified by column chromatography using 100-200 silica and 30-40% ethyl acetate in pet ether as an eluent to afford the product (100 g, 37.0% yield) as a pale brown solid. LCMS: [M+H]⁺=226.09

### Step 4: 6-(4,4,5,5-tetramethyl-1,3,2-dioxaborolan-2-yl)-3,4-dihydroisoquinolin-1(2H)-one

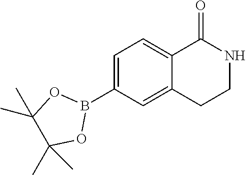 [SMILES: CC1(C)OB(c2ccc3c(c2)CCNC3=O)OC1(C)C]

To a stirred solution of 6-bromo-3,4-dihydroisoquinolin-1(2H)-one (10 g, 44.3 mmol) in 1,4-dioxane (100 mL) was added B₂Pin₂ (16.8 g, 66.4 mmol), KOAc (8.7 g, 88.5 mmol), and the mixture was bubbled with argon for 15 min at RT. Then Pd(dppf)Cl₂·DCM (1.85 g, 2.21 mmol) was added and the mixture was heated at 80° C. for 16 h before cooling to RT. The reaction was filtered through celite bed and washed with EtOAc (300 mL) and concentrated under reduced pressure to give the crude material which was purified by using 100-200 silica and EtOAc as an eluent to afford the product (10 g, 83.0%) as a grey solid. LCMS: 274.22 [M+H]⁺=274.22.

### Step 5: 2-6-difluoropyrazine

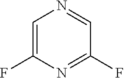

To a stirred solution of 2,6-dichloropyrazine (7 g, 47.2 mmol) in DMSO (35 mL) was added CsF (14.4 g, 94.5 mmol) and the mixture was heated in a sealed tube at 120° C. for 16 h before cooling to RT. The reaction mixture was poured into ice cold water and extracted with diethyl ether (2×100 mL). The organic layer was washed with sat. NaCl solution (2×100 mL). The organic layer was dried over Na₂SO₄ and concentrated under reduced pressure to afford product (4 g, 72%) as light-yellow liquid. TLC (10% EtOAc:pet ether) R_{f}=0.6.

### Step 6: tert-butyl(6-fluoropyrazin-2-yl)carbamate

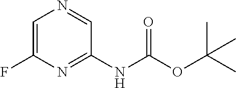

To a stirred solution of 2-6-difluoropyrazine (4 g, 30.3 mmol) was added tert-butylcarbamate (5.3 g, 45.4, mmol), Pd₂(dba)₃(0.8 g, 0.9 mmol), Xantphos (1 g, 1.8 mmol) and LiHMDS solution (40 mL, 10V, 1M in THF) in sealed tube and heated at 80° C. for 2h before cooling to RT. The reaction mixture was poured into ice cold water and extracted with EtOAc (2×100 mL). The organic layer was washed with sat. NaCl solution (2×100 mL), dried over Na₂SO₄ and concentrated under reduced pressure to give crude residue which was purified by column chromatography silica gel (100-200 mesh) as an eluent 0-10% EtOAc in pet ether to afford the product (3.6 g, 56%) as an off white solid. LCMS: [M+H]⁺=214.13

### Step 7: 6-fluoropyrazin-2-amine

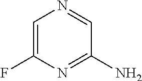

To a stirred solution of tert-butyl(6-fluoropyrazin-2-yl)carbamate (20 g, 93.9 mmol) in DCM (200 mL) was added trifluoro acetic acid (40 mL, 2V) at 0° C. to RT for 16 h. The reaction mixture was basified with sat. NaHCO₃ solution (300 mL) and extracted with EtOAc (2×500 mL). The combined organic layer was dried over Na₂SO₄ and concentrated under reduced pressure to afford the product (8 g, 75%) as a brown solid. LCMS: [M+H]⁺=113.9.

### Step 8: 5-bromo-6-fluoropyrazin-2-amine

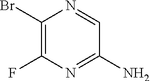

To a solution of 6-fluoropyrazin-2-amine (7.5 g, 66.3 mmol) in ACN (750 mL), was added NBS (11.8 g, 66.3 mmol) portionwise at 0° C. and the mixture was stirred for 10 min. The mixture was poured into ice cold water and extracted with EtOAc (2×500 mL). The combined organic layers were washed with sat. NaCl solution (2×500 mL). The organic layer was dried over Na₂SO₄ and concentrated under reduced pressure to give crude residue which was purified by column chromatography (silica gel, 100-200 mesh), using 0 to 20% EtOAc in pet ether as an eluent to afford the product (8 g, 63%) as a pale yellow solid. LCMS: [M+H]⁺: =191.95

### Step 9: 5-bromo-6-fluoro-3-iodopyrazin-2-amine

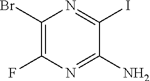

To a solution of 5-bromo-6-fluoropyrazin-2-amine (5.5 g, 28.6 mmol) in DMF (60 mL), was added N-iodo succinimide (19.3 g, 85.9 mmol), and the mixture was stirred at 80° C. for 6 h before cooling to RT. The mixture was poured into ice cold water and extracted with EtOAc (2×300 mL). The organic layer was washed with sat. NaCl solution (2×200 mL). The organic layer was dried over Na₂SO₄ and concentrated under reduced pressure to give crude residue. Which was purified by column chromatography (silicagel, 100-200 mesh), using 0 to 20% EtOAc in pet ether as an eluent to afford the product (3.2 g, 60%) as a pale-yellow solid. LCMS: [M+H]⁺=315.96.

### Step 10: 6-(3-amino-6-bromo-5-fluoropyrazin-2-yl)-3,4-dihydroisoquinolin-1(2H)-one

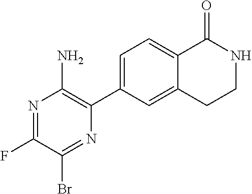 [SMILES: Nc1nc(F)c(Br)nc1-c1ccc2c(c1)CCNC2=O]

To a degassed suspension of 6-(4,4,5,5-tetramethyl-1,3,2-dioxaborolan-2-yl)-3,4-dihydroisoquinolin-1(2H)-one (5.6 g, 2.8 mmol) in 1,4-dioxane (100 mL) was added 5-bromo-6-fluoro-3-iodopyrazin-2-amine (5.5 g, 17.3 mmol), 2M Na₂CO₃ (17.3 mL, 34.7 mmol), degassed with argon for 10 min, was added Pd(dppf)Cl₂·DCM (0.7 g, 0.8 mmol) and then, the reaction mixture was heated to 90° C. for 16h before cooling to RT. The reaction mixture was filtered through a celite bed, which was washed with ethyl acetate (200 mL). Then, the filtrate was concentrated under reduced pressure. The obtained residue was purified by column chromatography (silica gel, 100-200 mesh) using as an eluent 100% EtOAc to afford (5 g, 86%) as a yellow solid. LCMS: [M+H]+=337.02.

### Step 11: tert-butyl (S)-2-(5-(5-amino-3-fluoro-6-(1-oxo-1,2,3,4-tetrahydroisoquinolin-6-yl)pyrazin-2-yl)-2-(tetrahydro-2H-pyran-4-yl)phenyl)pyrrolidine-1-carboxylate

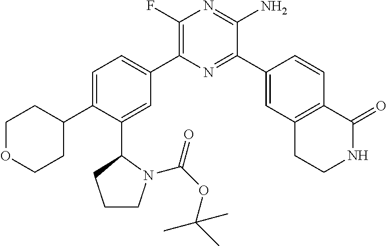 [SMILES: CC(C)(C)OC(=O)N1CCC[C@H]1c1cc(-c2nc(-c3ccc4c(c3)CCNC4=O)c(N)nc2F)ccc1C1CCOCC1]

A mixture of 6-(3-amino-6-bromo-5-fluoropyrazin-2-yl)-3,4-dihydroisoquinolin-1(2H)-one (30. mg, 0.089 mmol), tert-butyl (S)-2-(2-(tetrahydro-2H-pyran-4-yl)-5-(4,4,5,5-tetramethyl-1,3,2-dioxaborolan-2-yl)phenyl)pyrrolidine-1-carboxylate (54.3 mg, 0.107 mmol), [1,1′-Bis(diphenylphosphino)ferrocene]dichloropalladium(II) (9.77 mg, 0.013 mmol) and Cs₂CO₃ (87 mg, 0.267 mmol) in DME (4 mL) was flushed with argon. Water (1 mL) was added and the reaction mixture was heated in a microwave reactor at 90° C. for 1.5 h. The reaction mixture was partitioned between water (3 mL) and EtOAc (3 mL), the organic phase was separated, the aqueous phase was extracted with EtOAc (2×3 ml), the combined organic layers were washed with brine (3 mL), dried over Na₂SO₄, concentrated onto Celite® and purified by column chromatography (0-100% EtOAc in hexanes) to yield the title compound as a tan colored solid (42 mg, 91%). LCMS: [M+1]+=588.2

### Step 12: (S)-6-(3-amino-5-fluoro-6-(3-(pyrrolidin-2-yl)-4-(tetrahydro-2H-pyran-4-yl)phenyl)pyrazin-2-yl)-3,4-dihydroisoquinolin-1(2H)-one

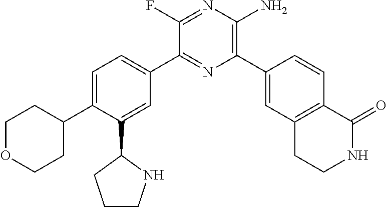 [SMILES: Nc1nc(F)c(-c2ccc(C3CCOCC3)c([C@@H]3CCCN3)c2)nc1-c1ccc2c(c1)CCNC2=O]

To a solution of tert-butyl (S)-2-(5-(5-amino-3-fluoro-6-(1-oxo-1,2,3,4-tetrahydroisoquinolin-6-yl)pyrazin-2-yl)-2-(tetrahydro-2H-pyran-4-yl)phenyl)pyrrolidine-1-carboxylate (50 mg, 0.085 mmol) in DCM (2 mL), was added TFA (0.586 mL, 7.66 mmol) at RT and the reaction mixture was continuously stirred at RT for 1 h. The mixture was concentrated and the residue was dissolved in MeOH and purified by catch and release method on a porapak cation exchange resin cartridge to isolate the title compound as a beige solid (12 mg, 28%). ¹H NMR (500 MHz, METHANOL-d₄ δ 7.94-8.01 (m, 2H), 7.83-7.89 (m, 1H), 7.86 (d, J=8.2 Hz, 1H), 7.63-7.68 (m, 1H), 7.60 (s, 1H), 7.42 (d, J=8.3 Hz, 1H), 4.93 (t, J=8.1 Hz, 1H), 3.94-4.02 (m, 2H), 3.55 (t, J=11.1 Hz, 2H), 3.47 (t, J=6.7 Hz, 2H), 3.28-3.40 (m, 2H), 3.05-3.14 (m, 1H), 2.93-3.03 (m, 2H), 2.35-2.46 (m, 1H), 2.08-2.27 (m, 3H), 1.89 (dd, J=12.5, 4.10 Hz, 1H), 1.70-1.80 (m, 1H), 1.65 (d, J=13.3 Hz, 1H), 1.59 (d, J=13.2 Hz, 1H). LCMS: [M+H]⁺=488.39. HPLC purity >95%.

Additional examples are prepared using methods and procedures similar to those described above.

Compounds of Formula I-18 to I-40 generally can be prepared according to the processes illustrated in the Schemes above and illustrated in Examples I to 18.

### Example 19: (S)-6-(3-amino-5-fluoro-6-(3-(pyrrolidin-2-yl)-4-(tetrahydro-2H-pyran-4-yl)phenyl)pyrazin-2-yl)-7-fluoro-3,4-dihydroisoquinolin-1(2H)-one (I-19)

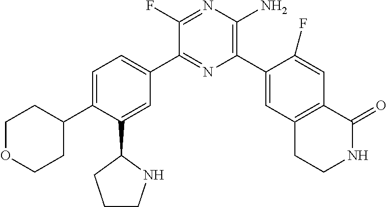 [SMILES: Nc1nc(F)c(-c2ccc(C3CCOCC3)c([C@@H]3CCCN3)c2)nc1-c1cc2c(cc1F)C(=O)NCC2]

### Step 1: tert-butyl (S)-2-(5-(5-amino-3-fluoro-6-(7-fluoro-1-oxo-1,2,3,4-tetrahydroisoquinolin-6-yl)pyrazin-2-yl)-2-(tetrahydro-2H-pyran-4-yl)phenyl)pyrrolidine-1-carboxylate

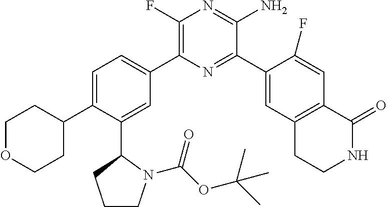 [SMILES: CC(C)(C)OC(=O)N1CCC[C@H]1c1cc(-c2nc(-c3cc4c(cc3F)C(=O)NCC4)c(N)nc2F)ccc1C1CCOCC1]

Prepared from 6-(3-amino-6-bromo-5-fluoropyrazin-2-yl)-7-fluoro-3,4-dihydroisoquinolin-1(2H)-one (32 mg, 0.090 mmol) and tert-butyl (S)-2-(2-(tetrahydro-2H-pyran-4-yl)-5-(4,4,5,5-tetramethyl-1,3,2-dioxaborolan-2-yl)phenyl)pyrrolidine-1-carboxylate (55.0 mg, 0.108 mmol) using a procedure similar to the previous Example to give the product as a beige solid (12 mg, 22% yield). LCMS: [M+H]+=605.69

### Step 2: (S)-6-(3-amino-5-fluoro-6-(3-(pyrrolidin-2-yl)-4-(tetrahydro-2H-pyran-4-yl)phenyl)pyrazin-2-yl)-7-fluoro-3, 4-dihydroisoquinolin-1(2H)-one

Prepared in a manner previous to the last step of the previous Example from tert-butyl-(S)-2-(5-(5-amino-3-fluoro-6-(7-fluoro-1-oxo-1,2,3,4-tetrahydroisoquinolin-6-yl)pyrazin-2-yl)-2-(tetrahydro-2H-pyran-4-yl)phenyl)pyrrolidine-1-carboxylate (12 mg, 0.020 mmol) and TFA to give the title compound was isolated as a beige solid (8 mg, 76% yield). 1H NMR (500 MHz, METHANOL-d4) δ ppm 8.00-8.05 (m, 1H), 7.91-7.97 (m, 1H), 7.79 (d, J=10.15 Hz, 1H), 7.54 (d, J=7.95 Hz, 2H), 5.01-5.11 (m, 1H), 4.05-4.14 (m, 2H), 3.65-3.72 (m, 2H), 3.56-3.61 (m, 2H), 3.42-3.54 (m, 2H), 3.17-3.27 (m, 1H), 3.06 (t, J=6.60 Hz, 2H), 2.46-2.56 (m, 1H), 2.19-2.35 (m, 3H), 1.98-2.02 (m, 1H), 2.00 (dd, J=12.59, 4.16 Hz, 1H), 1.82-1.91 (m, 1H), 1.77 (d, J=12.96 Hz, 1H), 1.67-1.73 (m, 1H); LCMS: [M+H]+=506.32.

### Example 20: 6-(3-amino-5-fluoro-6-(4-(4-isopropylpiperazin-1-yl)phenyl)pyrazin-2-yl)isoquinolin-1(2H)-one (I-20)

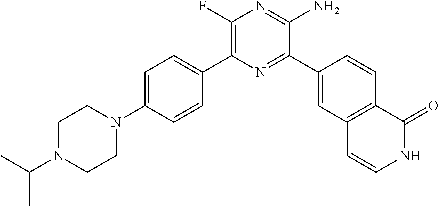 [SMILES: CC(C)N1CCN(c2ccc(-c3nc(-c4ccc5c(=O)[nH]ccc5c4)c(N)nc3F)cc2)CC1]

### Step 1: 6-(3-amino-6-bromo-5-fluoropyrazin-2-yl)isoquinolin-1(2H)-one

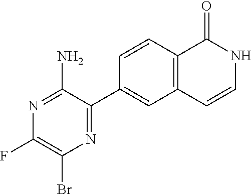 [SMILES: Nc1nc(F)c(Br)nc1-c1ccc2c(=O)[nH]ccc2c1]

To a solution of 5-bromo-6-fluoro-3-iodopyrazin-2-amine (53 mg, 0.150 mmol) and 6-(tetramethyl-1,3,2-dioxaborolan-2-yl)-2H-isoquinolin-1-one (44.8 mg, 0.165 mmol) in acetonitrile (4 ml) was added a solution of sodium carbonate monohydrate (39.1 mg, 0.315 mmol) in water (1 ml). The system was degassed and bis(triphenylphosphine)palladium(II) dichloride (12.64 mg, 0.018 mmol) was added. The reaction was flushed with nitrogen and heated on the stir plate at 80° C. overnight. The reaction was concentrated onto celite and purified by silica gel chromatography eluting with 0-100% EtOAc/Hexanes to give the product (25.2 mg, 50% yield) as a beige solid. LCMS: [M+H]⁺=335.11.

### Step 2: 6-(3-amino-5-fluoro-6-(4-(4-isopropylpiperazin-1-yl)phenyl)pyrazin-2-yl)isoquinolin-1(2H)-one

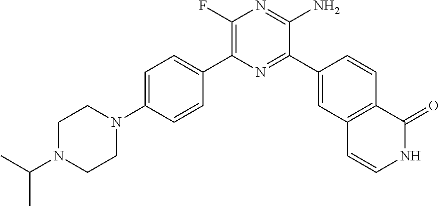 [SMILES: CC(C)N1CCN(c2ccc(-c3nc(-c4ccc5c(=O)[nH]ccc5c4)c(N)nc3F)cc2)CC1]

To a suspension of 6-(3-amino-6-bromo-5-fluoropyrazin-2-yl)isoquinolin-1(2H)-one (25 mg, 0.075 mmol), 4-(4-isopropylpiperazinyl)phenylboronic acid, pinacol ester (29.6 mg, 0.090 mmol) and cesium carbonate (60.8 mg, 0.186 mmol) in a 3:1 mixture of 1,2-dimethoxyethane (DME) (4 ml):water (1.3 ml) was added PdCl₂dppf (5.46 mg, 7.46 μmol). The mixture was flushed with nitrogen and heated at 90° C. on the stir plate for 1 hour. The reaction was concentrated onto celite and purified by silica gel chromatography eluting with 0-10% MeOH/DCM+1% NH₄OH, followed by reverse phase chromatography (C18, 0-100% ACN/H₂O). The desired fractions were then passed through an Isolute SCX-2 cation exchange resin cartridge eluting with 3% NH₃ in MeOH to give the title compound (8.1 mg, 23.7% yield) as a bright yellow solid. ¹H NMR (500 MHz, DMSO-d₆) δ ppm 11.27 (d, J=4.65 Hz, 1H), 8.28 (d, J=8.31 Hz, 1H), 7.99 (s, 1H), 7.81 (d, J=8.31 Hz, 1H), 7.76 (d, J=8.19 Hz, 2H), 7.21 (t, J=6.42 Hz, 1H), 7.01 (d, J=8.68 Hz, 2H), 6.77 (br. s., 2H), 6.64 (d, J=6.97 Hz, 1H), 3.15-3.23 (m, 4H), 2.66-2.74 (m, 1H), 2.55-2.64 (m, 4H), 1.02 (d, J=4.52 Hz, 6H); LCMS: [M+H]⁺=459.41.

### Example 21: 6-(3-amino-5-fluoro-6-(4-(4-isopropylpiperazin-1-yl)phenyl)pyrazin-2-yl)-8-fluoroisoquinolin-1(2H)-one (I-21)

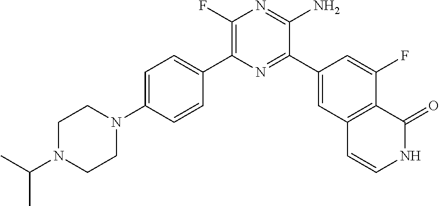 [SMILES: CC(C)N1CCN(c2ccc(-c3nc(-c4cc(F)c5c(=O)[nH]ccc5c4)c(N)nc3F)cc2)CC1]

### Step 1: (8-fluoro-1-oxo-1,2-dihydroisoquinolin-6-yl)boronic acid

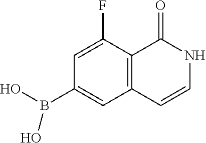 [SMILES: O=c1[nH]ccc2cc(B(O)O)cc(F)c12]

Prepared from 6-bromo-8-fluoro-1,2-dihydroisoquinolin-1-one (400 mg, 1.653 mmol) to give the boronic acid which was used in the next step without further purification. LCMS: [M+H]⁺=208.28.

### Step 2: 6-(3-amino-6-bromo-5-fluoropyrazin-2-yl)-8-fluoroisoquinolin-1(2H)-one

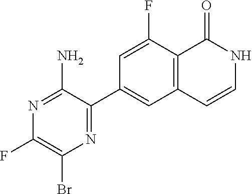 [SMILES: Nc1nc(F)c(Br)nc1-c1cc(F)c2c(=O)[nH]ccc2c1]

Prepared from 5-bromo-6-fluoro-3-iodopyrazin-2-amine (350 mg, 1.101 mmol) and (8-fluoro-1-oxo-1,2-dihydroisoquinolin-6-yl)boronic acid (296 mg, 1.431 mmol) to give the product (149 mg, 38.3% yield) as a beige solid. LCMS: [M+H]⁺=353.29.

### Step 3: 6-(3-amino-5-fluoro-6-(4-(4-isopropylpiperazin-1-yl)phenyl)pyrazin-2-yl)-8-fluoroisoquinolin-1(2H)-one

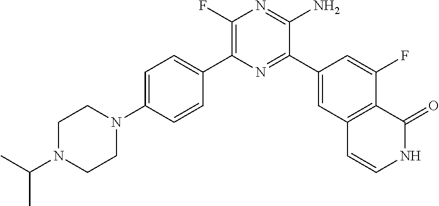 [SMILES: CC(C)N1CCN(c2ccc(-c3nc(-c4cc(F)c5c(=O)[nH]ccc5c4)c(N)nc3F)cc2)CC1]

Prepared from 6-(3-amino-6-bromo-5-fluoropyrazin-2-yl)-8-fluoroisoquinolin-1(2H)-one (55 mg, 0.156 mmol) and 4-(4-isopropylpiperazinyl)phenylboronic acid, pinacol ester (61.7 mg, 0.187 mmol) to give the title compound (34.7 mg, 46.8% yield) as a yellow solid. ¹H NMR (500 MHz, DMSO-d₆) δ ppm 11.27 (br. s., 1H), 7.80 (s, 1H), 7.77 (d, J=8.19 Hz, 2H), 7.48 (d, J=12.47 Hz, 1H), 7.22 (t, J=5.93 Hz, 1H), 7.04 (d, J=7.95 Hz, 2H), 6.87 (br. s., 2H), 6.62 (d, J=6.85 Hz, 1H), 3.21 (br. s., 4H), 2.85-3.00 (m, 1H), 2.64 (br. s., 4H), 1.07 (br. s., 6H); LCMS: [M+H]⁺=477.58.

### Example 22: 6-(3-amino-5-fluoro-6-(4-(4-isopropylpiperazin-1-yl)phenyl)pyrazin-2-yl)-7-fluoroisoquinolin-1(2H)-one (I-22)

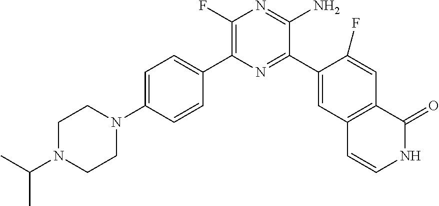 [SMILES: CC(C)N1CCN(c2ccc(-c3nc(-c4cc5cc[nH]c(=O)c5cc4F)c(N)nc3F)cc2)CC1]

Prepared from 6-(3-amino-6-bromo-5-fluoropyrazin-2-yl)-7-fluoroisoquinolin-1(2H)-one (55 mg, 0.156 mmol) and 4-(4-isopropylpiperazinyl)phenylboronic acid, pinacol ester (61.7 mg, 0.187 mmol) to give 6-(3-amino-5-fluoro-6-(4-(4-isopropylpiperazin-1-yl)phenyl)pyrazin-2-yl)-7-fluoroisoquinolin-1(2H)-one (43.7 mg, 0.092 mmol, 58.9% yield) (43.7 mg) as a yellow solid. ¹H NMR (500 MHz, DMSO-d₆) δ ppm 11.41 (d, J=4.65 Hz, 1H), 7.94 (d, J=10.39 Hz, 1H), 7.90 (d, J=6.72 Hz, 1H), 7.72 (d, J=8.19 Hz, 2H), 7.21 (t, J=6.24 Hz, 1H), 7.02 (d, J=7.34 Hz, 2H), 6.74 (br. s., 2H), 6.66 (d, J=6.97 Hz, 1H), 3.17 (d, J=4.28 Hz, 4H), 2.88-3.02 (m, 1H), 2.52-2.85 (m, 4H), 1.09 (br. s., 6H); LCMS: [M+H]⁺=477.33.

### Example 23: 6-(3-amino-6-(4-(4-(cyclopropylmethyl)piperazin-1-yl)phenyl)-5-fluoropyrazin-2-yl)isoquinolin-1(2H)-one (I-23)

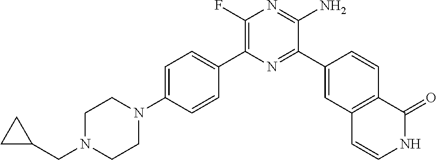 [SMILES: Nc1nc(F)c(-c2ccc(N3CCN(CC4CC4)CC3)cc2)nc1-c1ccc2c(=O)[nH]ccc2c1]

### Step 1: 6-(3-amino-6-bromo-5-fluoropyrazin-2-yl)isoquinolin-1(2H)-one

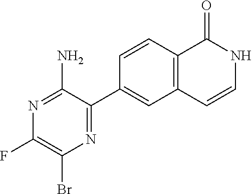 [SMILES: Nc1nc(F)c(Br)nc1-c1ccc2c(=O)[nH]ccc2c1]

Prepared from 5-bromo-6-fluoro-3-iodopyrazin-2-amine (53 mg, 0.150 mmol) and 6-(tetramethyl-1,3,2-dioxaborolan-2-yl)-2H-isoquinolin-1-one (44.8 mg, 0.165 mmol) to give the product (25.2 mg, 50.1% yield) as a beige solid. LCMS: [M+H]⁺=335.11.

### Step 2: 1-(4-bromophenyl)-4-(cyclopropylmethyl)piperazine

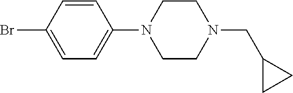 [SMILES: Brc1ccc(N2CCN(CC3CC3)CC2)cc1]

To a solution of 1-(4-bromophenyl)piperazine (1781 mg, 7.39 mmol) and N,N-diisopropylethylamine (6.43 ml, 36.9 mmol) in N,N-dimethylformamide (10 ml) was added (bromomethyl)cyclopropane (1.433 mL, 14.77 mmol) and the mixture was stirred at RT overnight. The mixture was diluted with water and extracted with DCM (3×). The combined organic layers were washed with brine, dried over anhydrous MgSO₄ and concentrated onto celite. The crude material was purified by flash sgc eluting with 0-100% EtOAc/Hexanes. The desired fractions were collected, concentrated and dried under vacuum to afford the product (2038 mg, 93% yield) as an off-white solid. LCMS: [M+H]⁺=295.37.

### Step 3: 1-(cyclopropylmethyl)-4-(4-(4,4,5,5-tetramethyl-1,3,2-dioxaborolan-2-yl)phenyl)piperazine

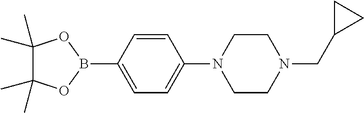 [SMILES: CC1(C)OB(c2ccc(N3CCN(CC4CC4)CC3)cc2)OC1(C)C]

Prepared from 1-(4-bromophenyl)-4-(cyclopropylmethyl)piperazine (1.88 g, 6.37 mmol) to give the boronate which was used in the next step without further purification. LCMS: [M+H]+=343.58.

### Step 4: 6-(3-amino-6-(4-(4-(cyclopropylmethyl)piperazin-1-yl)phenyl)-5-fluoropyrazin-2-yl)isoquinolin-1(2H)-one

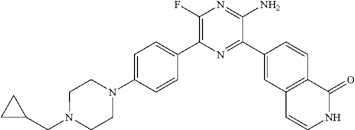 [SMILES: Nc1nc(F)c(-c2ccc(N3CCN(CC4CC4)CC3)cc2)nc1-c1ccc2c(=O)[nH]ccc2c1]

Prepared from 6-(3-amino-6-bromo-5-fluoropyrazin-2-yl)isoquinolin-1(2H)-one (60 mg, 0.179 mmol) and 1-(cyclopropylmethyl)-4-(4-(4,4,5,5-tetramethyl-1,3,2-dioxaborolan-2-yl)phenyl)piperazine (73.5 mg, 0.215 mmol) to give the product (18.3 mg, 21.7% yield) as a yellow solid. ¹H NMR (500 MHz, DMSO-d₆) δ ppm 11.27 (d, J=4.65 Hz, 1H), 8.28 (d, J=8.31 Hz, 1H), 7.99 (s, 1H), 7.81 (dd, J=8.31, 1.22 Hz, 1H), 7.76 (d, J=8.07 Hz, 2H), 7.21 (t, J=6.42 Hz, 1H), 7.03 (d, J=8.68 Hz, 2H), 6.78 (br. s., 2H), 6.64 (d, J=6.97 Hz, 1H), 3.22 (br. s., 4H), 2.60 (br. s., 2H), 2.25 (br. s., 2H), 0.84-0.94 (m, 1H), 0.49 (d, J=5.75 Hz, 2H), 0.12 (br. s., 2H); LCMS: [M+H]⁺=471.57.

### Example 24: 6-(3-amino-6-(4-(4-(cyclopropylmethyl)piperazin-1-yl)phenyl)-5-fluoropyrazin-2-yl)-8-fluoroisoquinolin-1(2H)-one (I-24)

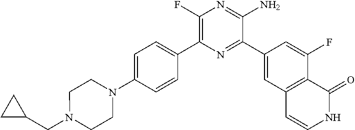 [SMILES: Nc1nc(F)c(-c2ccc(N3CCN(CC4CC4)CC3)cc2)nc1-c1cc(F)c2c(=O)[nH]ccc2c1]

Prepared from 6-(3-amino-6-bromo-5-fluoropyrazin-2-yl)-8-fluoroisoquinolin-1(2H)-one (40 mg, 0.113 mmol) and 1-(cyclopropylmethyl)-4-(4-(4,4,5,5-tetramethyl-1,3,2-dioxaborolan-2-yl)phenyl)piperazine (46.5 mg, 0.136 mmol) to give the title compound (9.6 mg, 36.6% yield) as a yellow solid. ¹H NMR (500 MHz, DMSO-d₆) δ ppm 11.27 (br. s., 1H), 7.80 (br. s., 1H), 7.77 (d, J=8.07 Hz, 2H), 7.48 (d, J=12.47 Hz, 1H), 7.22 (t, J=5.99 Hz, 1H), 7.04 (d, J=7.95 Hz, 2H), 6.87 (br. s., 2H), 6.62 (d, J=6.60 Hz, 1H), 3.14-3.28 (m, 4H), 2.63 (br. s., 4H), 2.36 (br. s., 2H), 0.91 (br. s., 1H), 0.52 (br. s., 2H), 0.15 (br. s., 2H); LCMS: [M+H]⁺=489.49.

### Example 25: 6-(3-amino-6-(4-(4-(cyclopropylmethyl)piperazin-1-yl)phenyl)-5-fluoropyrazin-2-yl)-7-fluoroisoquinolin-1(2H)-one (I-25)

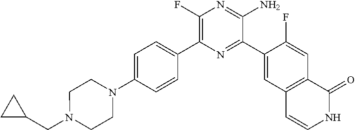 [SMILES: Nc1nc(F)c(-c2ccc(N3CCN(CC4CC4)CC3)cc2)nc1-c1cc2cc[nH]c(=O)c2cc1F]

### Step 1: (7-fluoro-1-oxo-1,2-dihydroisoquinolin-6-yl)boronic acid

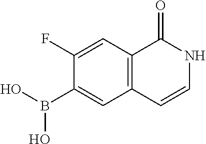 [SMILES: O=c1[nH]ccc2cc(B(O)O)c(F)cc12]

Prepared from 6-bromo-7-fluoro-2H-isoquinolin-1-one (360 mg, 1.487 mmol) to give the boronic acid which was used in the next step without further purification. LCMS: [M−H]⁻=206.34.

### Step 2: 6-(3-amino-6-bromo-5-fluoropyrazin-2-yl)-7-fluoroisoquinolin-1(2H)-one

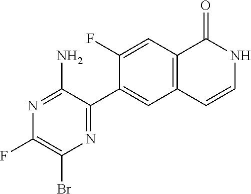 [SMILES: Nc1nc(F)c(Br)nc1-c1cc2cc[nH]c(=O)c2cc1F]

Prepared from 5-bromo-6-fluoro-3-iodopyrazin-2-amine (300 mg, 0.944 mmol) and (7-fluoro-1-oxo-1,2-dihydroisoquinolin-6-yl)boronic acid (234 mg, 1.132 mmol) to give the product (113 mg, 33.9% yield) as a beige solid. LCMS: [M+H]⁺=353.29.

### Step 3: 6-(3-amino-6-(4-(4-(cyclopropylmethyl)piperazin-1-yl)phenyl)-5-fluoropyrazin-2-yl)-7-fluoroisoquinolin-1(2H)-one

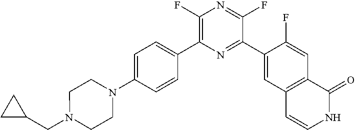 [SMILES: O=c1[nH]ccc2cc(-c3nc(-c4ccc(N5CCN(CC6CC6)CC5)cc4)c(F)nc3F)c(F)cc12]

Prepared from 6-(3-amino-6-bromo-5-fluoropyrazin-2-yl)-7-fluoroisoquinolin-1(2H)-one (30 mg, 0.085 mmol) and 1-(cyclopropylmethyl)-4-(4-(4,4,5,5-tetramethyl-1,3,2-dioxaborolan-2-yl)phenyl)piperazine (34.9 mg, 0.102 mmol) to give the title compound (5.9 mg, 14.2% yield) as a yellow solid. ¹H NMR (500 MHz, DMSO-d₆) δ ppm 11.41 (d, J=5.38 Hz, 1H), 7.88-7.95 (m, 2H), 7.71 (d, J=7.95 Hz, 2H), 7.17-7.24 (m, 1H), 7.00 (d, J=8.93 Hz, 2H), 6.72 (s, 2H), 6.64-6.68 (m, 1H), 3.21 (br. s., 4H), 2.60 (br. s., 4H), 2.23 (d, J=14.92 Hz, 2H), 0.86 (d, J=3.67 Hz, 1H), 0.49 (d, J=7.21 Hz, 2H), 0.11 (br. s., 2H); LCMS: [M+H]⁺=489.56.

### Example 26: 6-(3-amino-6-(4-(4-(2-cyclopropylethyl)piperazin-1-yl)phenyl)-5-fluoropyrazin-2-yl)-8-fluoro-3,4-dihydroisoquinolin-1(2H)-one (I-26)

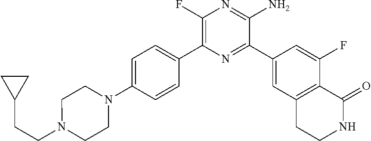 [SMILES: Nc1nc(F)c(-c2ccc(N3CCN(CCC4CC4)CC3)cc2)nc1-c1cc(F)c2c(c1)CCNC2=O]

### Step 1: (8-fluoro-1-oxo-1,2,3,4-tetrahydroisoquinolin-6-yl)boronic acid

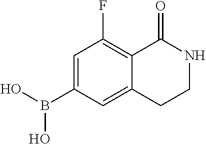 [SMILES: O=C1NCCc2cc(B(O)O)cc(F)c21]

Prepared from 6-bromo-8-fluoro-3,4-dihydroisoquinolin-1(2H)-one (1.5 g, 6.15 mmol) to give a mixture of the boronate and boronic acid which was used in the next step without further purification. LCMS: [M+H]⁺=292.23 Boronate, 210.29 Boronic acid.

### Step 2: 6-(3-amino-6-bromo-5-fluoropyrazin-2-yl)-8-fluoro-3,4-dihydroisoquinolin-1(2H)-one

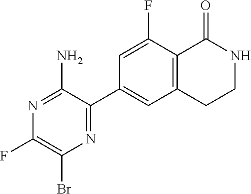 [SMILES: Nc1nc(F)c(Br)nc1-c1cc(F)c2c(c1)CCNC2=O]

Prepared from 5-bromo-6-fluoro-3-iodopyrazin-2-amine (350 mg, 0.991 mmol) and (8-fluoro-1-oxo-1,2,3,4-tetrahydroisoquinolin-6-yl)boronic acid (269 mg, 1.288 mmol) to give 6-(3-amino-6-bromo-5-fluoropyrazin-2-yl)-8-fluoro-3,4-dihydroisoquinolin-1(2H)-one (269 mg, 0.757 mmol, 76% yield) as a beige solid. LCMS: [M+H]⁺=355.24.

### Step 3: 1-(4-bromophenyl)-4-(2-cyclopropylethyl)piperazine

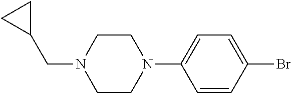 [SMILES: Brc1ccc(N2CCN(CC3CC3)CC2)cc1]

To a solution of 1-(4-bromophenyl)piperazine (205 mg, 0.850 mmol) and N,N-diisopropylethylamine (0.740 ml, 4.25 mmol) in N,N-dimethylformamide (2 ml) was added (2-bromo-ethyl)-cyclopropane (152 mg, 1.020 mmol). The reaction was stirred at RT over 4 days. The mixture was diluted with water and extracted with DCM (3×). The combined organic layers were washed with brine, dried over anhydrous MgSO₄, concentrated and dried under vacuum to obtain the product (261 mg, 99% yield) as a white solid. The material was used as-is in the next step without further purification. LCMS: [M+H]⁺=309.35.

### Step 4: 1-(2-cyclopropylethyl)-4-(4-(4,4,5,5-tetramethyl-1,3,2-dioxaborolan-2-yl)phenyl)piperazine

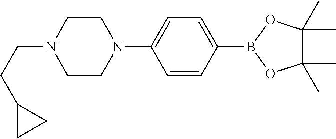 [SMILES: CC1(C)OB(c2ccc(N3CCN(CCC4CC4)CC3)cc2)OC1(C)C]

Prepared from 1-(4-bromophenyl)-4-(2-cyclopropylethyl)piperazine (83 mg, 0.268 mmol) to give the boronate which was used in the next step without further purification. LCMS: [M+H]⁺=357.37.

### Step 5: 6-(3-amino-6-(4-(4-(2-cyclopropylethyl)piperazin-1-yl)phenyl)-5-fluoropyrazin-2-yl)-8-fluoro-3,4-dihydroisoquinolin-1(2H)-one

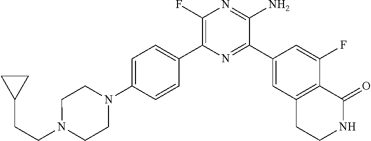 [SMILES: Nc1nc(F)c(-c2ccc(N3CCN(CCC4CC4)CC3)cc2)nc1-c1cc(F)c2c(c1)CCNC2=O]

Prepared from 6-(3-amino-6-bromo-5-fluoropyrazin-2-yl)-8-fluoro-3,4-dihydroisoquinolin-1(2H)-one (39 mg, 0.110 mmol) and 1-(2-cyclopropylethyl)-4-(4-(4,4,5,5-tetramethyl-1,3,2-dioxaborolan-2-yl)phenyl)piperazine (47.0 mg, 0.132 mmol) to give the product (3.8 mg, 6.9% yield) as a yellow solid. ¹H NMR (500 MHz, DMSO-d₆) δ ppm 8.03 (br. s., 1H), 7.74 (d, J=8.44 Hz, 2H), 7.49 (s, 1H), 7.42 (d, J=12.35 Hz, 1H), 7.01 (d, J=8.44 Hz, 2H), 6.82 (br. s., 2H), 3.19 (br. s., 4H), 2.97 (t, J=5.62 Hz, 2H), 2.40 (t, J=7.34 Hz, 2H), 1.37 (d, J=7.34 Hz, 2H), 0.70 (br. s., 1H), 0.40 (d, J=7.70 Hz, 2H), 0.04 (d, J=4.28 Hz, 2H); LCMS: [M+H]⁺=505.54.

### Example 27: 6-(3-amino-6-(4-(4-(2-cyclopropylethyl)piperazin-1-yl)phenyl)-5-fluoropyrazin-2-yl)-8-fluoroisoquinolin-1(2H)-one (I-27) (Prophetic)

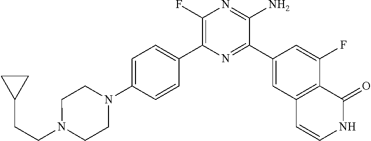 [SMILES: Nc1nc(F)c(-c2ccc(N3CCN(CCC4CC4)CC3)cc2)nc1-c1cc(F)c2c(=O)[nH]ccc2c1]

1-27 be prepared by processes described in Example 20, Step 2 from 6-(3-amino-6-bromo-5-fluoropyrazin-2-yl)-8-fluoroisoquinolin-1(2H)-one and 1-(2-cyclopropylethyl)-4-(4-(4,4,5,5-tetramethyl-1,3,2-dioxaborolan-2-yl)phenyl)piperazine.

### Example 28: 6-(3-amino-6-(4-(4-(2-cyclopropylethyl)piperazin-1-yl)phenyl)-5-fluoropyrazin-2-yl)-7-fluoroisoquinolin-1(2H)-one (I-28) (Prophetic)

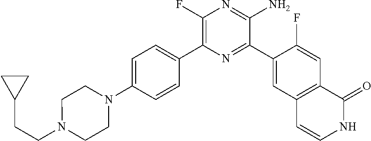 [SMILES: Nc1nc(F)c(-c2ccc(N3CCN(CCC4CC4)CC3)cc2)nc1-c1cc2cc[nH]c(=O)c2cc1F]

I-28 can be prepared by processes described in Example 20, Step 2 from 6-(3-amino-6-bromo-5-fluoropyrazin-2-yl)-7-fluoroisoquinolin-1(2H)-one and 1-(2-cyclopropylethyl)-4-(4-(4,4,5,5-tetramethyl-1,3,2-dioxaborolan-2-yl)phenyl)piperazine.

### Example 29: 6-(3-amino-6-(4-(4-(1-cyclopropylethyl)piperazin-1-yl)phenyl)-5-fluoropyrazin-2-yl)-8-fluoro-3,4-dihydroisoquinolin-1(2H)-one, Enantiomer 1 (I-29)

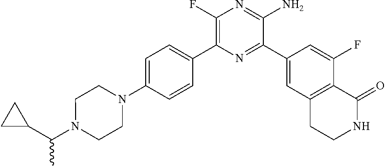 [SMILES: CC(C1CC1)N1CCN(c2ccc(-c3nc(-c4cc(F)c5c(c4)CCNC5=O)c(N)nc3F)cc2)CC1]

### Step 1: 8-fluoro-6-(4,4,5,5-tetramethyl-1,3,2-dioxaborolan-2-yl)-3,4-dihydroisoquinolin-1(2H)-one

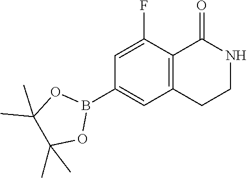 [SMILES: CC1(C)OB(c2cc(F)c3c(c2)CCNC3=O)OC1(C)C]

To a 100 mL round bottomed flask charged with 6-bromo-8-fluoro-3,4-dihydroisoquinolin-1(2H)-one (896 mg, 3.67 mmol), bis(pinacolato)diboron (1025 mg, 4.04 mmol) and potassium acetate (1081 mg, 11.01 mmol), was added anhydrous 1,4-dioxane (40 ml). The system was flushed with nitrogen then [1,1′-bis(diphenylphosphino)ferrocene]dichloropalladium(II) (269 mg, 0.367 mmol) was added. The mixture was further flushed with nitrogen then heated at 100° C. overnight. The reaction was diluted with acetonitrile, filtered through a pad of celite, and concentrated in vacuo to give a mixture of the boronate and boronic acid which was used in the next step without further purification. LCMS: [M+H]⁺=292.34 boronate, 210.39 boronic acid.

### Step 2: 6-(3-amino-6-bromo-5-fluoropyrazin-2-yl)-8-fluoro-3,4-dihydroisoquinolin-1(2H)-one

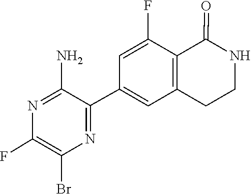 [SMILES: Nc1nc(F)c(Br)nc1-c1cc(F)c2c(c1)CCNC2=O]

To 5-bromo-6-fluoro-3-iodopyrazin-2-amine (760 mg, 2.391 mmol) and 8-fluoro-6-(4,4,5,5-tetramethyl-1,3,2-dioxaborolan-2-yl)-3,4-dihydroisoquinolin-1(2H)-one (835 mg, 2.87 mmol) in acetonitrile (15 ml) was added a solution of sodium carbonate monohydrate (623 mg, 5.02 mmol) in water (3.8 ml). The system was degassed and bis(triphenylphosphine)palladium(II) dichloride (201 mg, 0.287 mmol) was added. The reaction was flushed with nitrogen then heated 90° C. for 3 hours. The mixture was concentrated onto celite and purified by flash chromatography (Biotage, silica gel) eluting with 0-10% MeOH/DCM+1% NH₄OH to give 6-(3-amino-6-bromo-5-fluoropyrazin-2-yl)-8-fluoro-3,4-dihydroisoquinolin-1(2H)-one (536 mg, 1.509 mmol, 63.1% yield) as a beige solid. LCMS: [M+H]⁺=355.28.

### Step 3: (R)-1-(4-bromophenyl)-4-(1-cyclopropylethyl) piperazine and (S)-1-(4-bromophenyl)-4-(1-cyclopropylethyl) piperazine

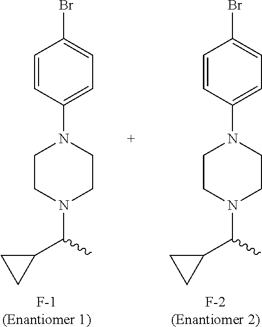

To a solution of 1-(4-bromophenyl) piperazine. HCl (3 g, 12.43 mmol) in methanol (60 mL) under nitrogen atmosphere glacial acetic acid (2 drops), sodium cyanoborohydride (1.9 g, 30.23 mmol) and 1-cyclopropylethan-1-one (2.6 g, 31.07 mmol) were added at room temperature and the reaction mass was stirred at 70° C. for 2.5 h. After 2.5 h, reaction mass cooled to room temperature and another portion of sodium cyanoborohydride (1.9 g, 30.23 mmol) and 1-cyclopropylethan-1-one (2.6 g, 31.07 mmol) were added and the reaction was further stirred at 70° C. for 16 h. After completion of the reaction, reaction was cooled to room temperature and water was added to it. The solid thus fallout was filtered, washed with water (50 mL) and hexanes (100 mL) and dried under vacuum to get the impure product. The impure product was further purified by crystallization with EtOAc to get mixture of (R)-1-(4-bromophenyl)-4-(1-cyclopropylethyl)piperazine and (S)-1-(4-bromophenyl)-4-(1-cyclopropylethyl)piperazine (1.7 g, 5.49 mmol, 44.15%). Both the isomers were separated by chiral SCF chromatography to get first fraction as F-1 (Enantiomer 1) (0.63 g, 2.03 mmol, 16.4%) and second fraction as F-2 (Enantiomer 2) (0.68 g, 2.19 mmol, 17.7%). F-1_1H NMR (400 MHz, DMSO-d₆) δ 7.34 (d, J=8.9 Hz, 2H), 6.89 (d, J=8.6 Hz, 2H), 3.11 (t, J=5.2 Hz, 4H), 2.75 (dt, J=10.6, 5.0 Hz, 2H), 2.63 (dt, J=10.8, 5.0 Hz, 2H), 1.71 (dt, J=13.1, 6.5 Hz, 1H), 1.08 (d, J=6.5 Hz, 3H), 0.74 (qt, J=8.6, 5.0 Hz, 1H), 0.54 (tt, J=8.9, 4.8 Hz, 1H), 0.40 (tt, J=8.9, 4.7 Hz, 1H), 0.27 (dq, J=9.8, 4.9 Hz, 1H). LCMS: [M+2H]⁺=309.0, 311.0. F-2_1H NMR (400 MHz, DMSO-d₆) 7.34 (d, J=8.9 Hz, 2H), 6.89 (d, J=8.6 Hz, 2H), 3.11 (t, J=5.2 Hz, 4H), 2.75 (dt, J=10.6, 5.0 Hz, 2H), 2.63 (dt, J=10.8, 5.0 Hz, 2H), 1.71 (dt, J=13.1, 6.5 Hz, 1H), 1.08 (d, J=6.5 Hz, 3H), 0.74 (qt, J=8.6, 5.0 Hz, 1H), 0.54 (tt, J=8.9, 4.8 Hz, 1H), 0.40 (tt, J=8.9, 4.7 Hz, 1H), 0.27 (dq, J=9.8, 4.9 Hz, 1H). LCMS: [M+H]⁺=309.0, 311.0

### Step 4: 1-(1-cyclopropylethyl)-4-(4-(4,4,5,5-tetramethyl-1,3,2-dioxaborolan-2-yl)phenyl)piperazine (Enantiomer 1)

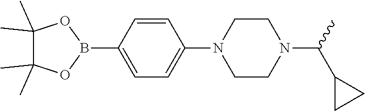 [SMILES: CC(C1CC1)N1CCN(c2ccc(B3OC(C)(C)C(C)(C)O3)cc2)CC1]

The title compound was prepared from (+)-1-(4-bromophenyl)-4-(1-cyclopropylethyl)piperazine, (enantiomer 1) (120 mg, 0.388 mmol) to give the boronate which was used in the next step without further purification. LCMS: [M+H]⁺ 3=57.24.

### Step 5: (+)-6-(3-amino-6-(4-(4-(1-cyclopropylethyl)piperazin-1-yl)phenyl)-5-fluoropyrazin-2-yl)-8-fluoro-3,4-dihydroisoquinolin-1(2H)-one (Enantiomer 1)

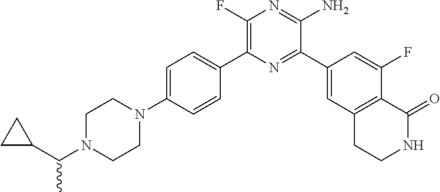 [SMILES: CC(C1CC1)N1CCN(c2ccc(-c3nc(-c4cc(F)c5c(c4)CCNC5=O)c(N)nc3F)cc2)CC1]

To a suspension of 6-(3-amino-6-bromo-5-fluoropyrazin-2-yl)-8-fluoro-3,4-dihydroisoquinolin-1(2H)-one (45 mg, 0.127 mmol), (1-(1-cyclopropylethyl)-4-(4-(4,4,5,5-tetramethyl-1,3,2-dioxaborolan-2-yl)phenyl)piperazine, unassigned enantiomer 1 (54.2 mg, 0.152 mmol), cesium carbonate (103 mg, 0.317 mmol) in a 3:1 mixture of 1,2-dimethoxyethane (4 ml):water (1.3 ml) was added [1,1′-bis(diphenylphosphino)ferrocene]dichloropalladium(II) (9.27 mg, 0.013 mmol). The system was flushed with nitrogen then heated at 90° C. overnight. The reaction was concentrated onto celite and purified by flash chromatography (Biotage, silica gel) eluting with 0-10% MeOH/DCM+1% NH₄OH, then re-purified on the Biotage (reverse phase silica gel) eluting with 0-100% ACN/H₂O. The reverse phase fractions were passed through a Biotage ISOLUTE Flash SCX-2 (2 g/15 ml) column washing with methanol, then eluting the compound with 3% NH₄OH in MeOH. The desired fractions were collected, concentrated and dried on the lyophilizer to give the title compound. Note: Unassigned single enantiomer. ¹H NMR (500 MHz, DMSO-d₆) δ ppm 8.03 (br. s., 1H), 7.75 (d, J=8.07 Hz, 2H), 7.50 (s, 1H), 7.43 (d, J=11.98 Hz, 1H), 7.02 (d, J=8.68 Hz, 2H), 6.82 (s, 2H), 3.36 (d, J=2.81 Hz, 2H), 3.20 (br. s., 4H), 2.97 (t, J=6.24 Hz, 2H), 2.78 (br. s., 2H), 2.62-2.71 (m, 2H), 1.74 (br. s., 1H), 1.10 (br. s., 3H), 0.76 (br. s., 1H), 0.56 (br. s., 1H), 0.41 (br. s., 1H), 0.28 (br. s., 1H), 0.03 (br. s., 1H); LCMS: [M+H]⁺=505.60.

### Example 30: 6-(3-amino-6-(4-(4-(1-cyclopropylethyl)piperazin-1-yl)phenyl)-5-fluoropyrazin-2-yl)-8-fluoro-3,4-dihydroisoquinolin-1(2H)-one, Enantiomer 2 (I-30)

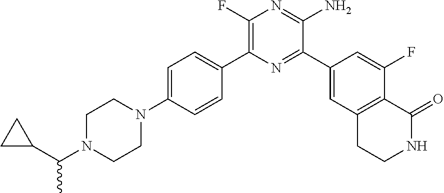 [SMILES: CC(C1CC1)N1CCN(c2ccc(-c3nc(-c4cc(F)c5c(c4)CCNC5=O)c(N)nc3F)cc2)CC1]

### Step 1: 1-(1-cyclopropylethyl)-4-(4-(4,4,5,5-tetramethyl-1,3,2-dioxaborolan-2-yl)phenyl)piperazine, enantiomer 2

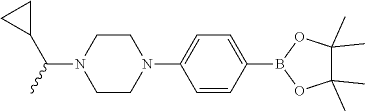 [SMILES: CC(C1CC1)N1CCN(c2ccc(B3OC(C)(C)C(C)(C)O3)cc2)CC1]

Prepared from F-2 1-(4-bromophenyl)-4-(1-cyclopropylethyl)piperazine, unassigned enantiomer 2 (120 mg, 0.388 mmol, preparation shown in Example 29) to give the boronate which was used in the next step without further purification. LCMS: [M+H]+=357.24.

### Step 2: 6-(3-amino-6-(4-(4-(1-cyclopropylethyl)piperazin-1-yl)phenyl)-5-fluoropyrazin-2-yl)-8-fluoro-3,4-dihydroisoquinolin-1(2H)-one, Enantiomer 2

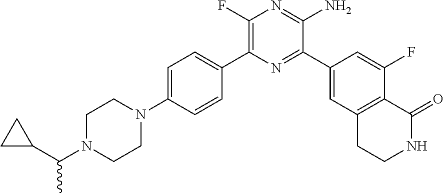 [SMILES: CC(C1CC1)N1CCN(c2ccc(-c3nc(-c4cc(F)c5c(c4)CCNC5=O)c(N)nc3F)cc2)CC1]

Prepared from 6-(3-amino-6-bromo-5-fluoropyrazin-2-yl)-8-fluoro-3,4-dihydroisoquinolin-1(2H)-one (45 mg, 0.127 mmol) and (−)-1-(1-cyclopropylethyl)-4-(4-(4,4,5,5-tetramethyl-1,3,2-dioxaborolan-2-yl)phenyl)piperazine (54.2 mg, 0.152 mmol) to give the title compound (21.1 mg, 33.0% yield) as a yellow solid. ¹H NMR (500 MHz, DMSO-d₆) δ ppm 8.03 (br. s., 1H), 7.75 (d, J=8.19 Hz, 2H), 7.50 (s, 1H), 7.43 (d, J=11.86 Hz, 1H), 7.02 (d, J=8.56 Hz, 2H), 6.82 (s, 2H), 3.35-3.38 (m, 2H), 3.19 (br. s., 4H), 2.97 (t, J=6.30 Hz, 2H), 2.78 (br. s., 2H), 2.62-2.71 (m, 2H), 1.74 (d, J=7.95 Hz, 1H), 1.10 (br. s., 3H), 0.77 (br. s., 1H), 0.56 (br. s., 1H), 0.42 (br. s., 1H), 0.29 (br. s., 1H), 0.05 (d, J=17.85 Hz, 1H); LCMS: [M+H]⁺=505.54.

### Example 31: 6-(3-amino-6-(4-(4-ethylpiperazin-1-yl)phenyl)-5-fluoropyrazin-2-yl)-8-fluoroisoquinolin-1(2H)-one (I-31) (Prophetic)

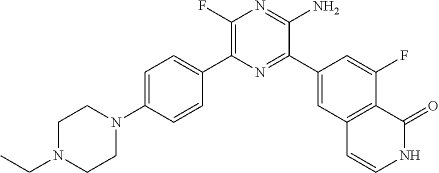 [SMILES: CCN1CCN(c2ccc(-c3nc(-c4cc(F)c5c(=O)[nH]ccc5c4)c(N)nc3F)cc2)CC1]

Compound 1-31 can be prepared following the process(es) of Example 20, Step 2 from 6-(3-amino-6-bromo-5-fluoropyrazin-2-yl)-8-fluoroisoquinolin-1(2H)-one and 1-ethyl-4-(4-(4,4,5,5-tetramethyl-1,3,2-dioxaborolan-2-yl)phenyl)piperazine.

### Example 32: 6-(3-amino-6-(4-(4-ethylpiperazin-1-yl)phenyl)-5-fluoropyrazin-2-yl)isoquinolin-1(2H)-one (I-32)

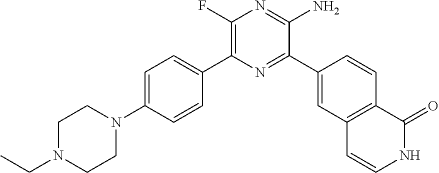 [SMILES: CCN1CCN(c2ccc(-c3nc(-c4ccc5c(=O)[nH]ccc5c4)c(N)nc3F)cc2)CC1]

Prepared from 6-(3-amino-6-bromo-5-fluoropyrazin-2-yl)isoquinolin-1(2H)-one (40 mg, 0.119 mmol) and 1-ethyl-4-(4-(4,4,5,5-tetramethyl-1,3,2-dioxaborolan-2-yl)phenyl)piperazine (45.3 mg, 0.143 mmol) to give 6-(3-amino-6-(4-(4-ethylpiperazin-1-yl)phenyl)-5-fluoropyrazin-2-yl)isoquinolin-1(2H)-one (23.7 mg, 44.7% yield) as a pale yellow solid. ¹H NMR (500 MHz, DMSO-d₆) δ ppm 11.27 (d, J=5.14 Hz, 1H), 8.27 (d, J=8.31 Hz, 1H), 7.99 (d, J=1.34 Hz, 1H), 7.81 (dd, J=8.31, 1.59 Hz, 1H), 7.76 (d, J=7.95 Hz, 2H), 7.21 (dd, J=6.97, 5.99 Hz, 1H), 7.02 (d, J=9.05 Hz, 2H), 6.77 (s, 2H), 6.64 (d, J=6.24 Hz, 1H), 3.18-3.24 (m, 4H), 2.45-2.49 (m, 2H), 2.35-2.39 (m, 2H), 1.04 (t, J=7.15 Hz, 3H); LCMS: [M+H]⁺=445.50.

### Example 33: 6-(3-amino-5-fluoro-6-(4-morpholinophenyl)pyrazin-2-yl)-3,4-dihydroisoquinolin-1(2H)-one (I-33)

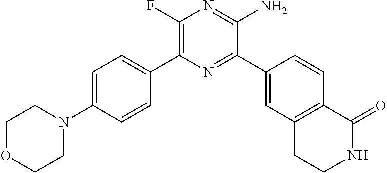 [SMILES: Nc1nc(F)c(-c2ccc(N3CCOCC3)cc2)nc1-c1ccc2c(c1)CCNC2=O]

To 6-(3-amino-6-chloro-5-fluoropyrazin-2-yl)-3,4-dihydroisoquinolin-1(2H)-one (80 mg, 0.273 mmol), 4-(morpholino)phenylboronic acid (85 mg, 0.410 mmol) and potassium phosphate tribasic (145 mg, 0.683 mmol) in a 8:1 mixture of 1,4-dioxane (9 ml):water (1.1 ml) was added XPhos Pd G2 (32.3 mg, 0.041 mmol). The system was degassed and heated at 90° C. overnight. The mixture was concentrated onto celite and purified by flash chromatography (Biotage, silica gel) eluting with 0-10% MeOH/DCM+1% NH₄OH. The desired fractions were collected, concentrated, triturated from methanol, and dried under vacuum to give the title compound (46.4 mg, 40.5% yield) as a yellow solid. ¹H NMR (500 MHz, DMSO-d₆) δ ppm 7.98 (br. s., 1H), 7.94 (d, J=7.70 Hz, 1H), 7.77 (d, J=7.95 Hz, 2H), 7.69 (d, J=7.82 Hz, 1H), 7.65 (s, 1H), 7.03 (d, J=7.70 Hz, 2H), 6.74 (br. s., 2H), 3.75 (br. s., 4H), 3.42 (br. s., 2H), 3.17 (br. s., 4H), 2.98 (br. s., 2H); LCMS: [M+H]⁺=420.55.

### Example 34: 6-(3-amino-5-fluoro-6-(4-morpholinophenyl)pyrazin-2-yl)-7-fluoro-3,4-dihydroisoquinolin-1(2H)-one (I-34)

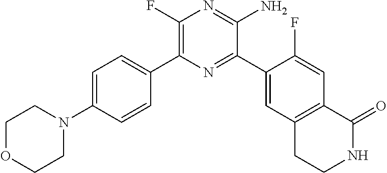 [SMILES: Nc1nc(F)c(-c2ccc(N3CCOCC3)cc2)nc1-c1cc2c(cc1F)C(=O)NCC2]

To 6-(3-amino-6-chloro-5-fluoropyrazin-2-yl)-7-fluoro-3,4-dihydroisoquinolin-1(2H)-one (70 mg, 0.225 mmol), 4-(morpholino)phenylboronic acid (70.0 mg, 0.338 mmol) and potassium phosphate tribasic (120 mg, 0.563 mmol) in a 8:1 mixture of 1,4-dioxane (4.5 ml):water (0.56 ml) was added XPhos Pd G2 (26.6 mg, 0.034 mmol). The system was degassed and heated at 90° C. overnight. The mixture was concentrated onto celite and purified by flash chromatography (Biotage, silica gel) eluting with 0-10% MeOH/DCM+1% NH₄OH. The desired fractions were collected, concentrated, triturated from methanol, and dried under vacuum to give the title compound (74.3 mg, 75% yield) as a yellow solid. ¹H NMR (500 MHz, DMSO-d₆) δ ppm 8.14 (br. s., 1H), 7.71 (d, J=8.19 Hz, 2H), 7.64 (d, J=10.03 Hz, 1H), 7.51 (d, J=6.36 Hz, 1H), 7.01 (d, J=8.56 Hz, 2H), 6.69 (br. s., 2H), 3.75 (br. s., 4H), 3.42 (br. s., 2H), 3.16 (br. s., 4H) 2.95 (br. s., 2H); LCMS: [M+H]⁺=438.54.

### Example 35: 6-(3-amino-5-fluoro-6-(4-morpholinophenyl)pyrazin-2-yl)-8-fluoro-3,4-dihydroisoquinolin-1(2H)-one (I-35)

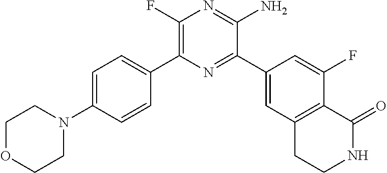 [SMILES: Nc1nc(F)c(-c2ccc(N3CCOCC3)cc2)nc1-c1cc(F)c2c(c1)CCNC2=O]

To a suspension of 6-(3-amino-6-bromo-5-fluoropyrazin-2-yl)-8-fluoro-3,4-dihydroisoquinolin-1(2H)-one (25 mg, 0.070 mmol), (R)-2-isopropyl-4-(4-(4,4,5,5-tetramethyl-1,3,2-dioxaborolan-2-yl)phenyl)morpholine (1.6 ml, 0.084 mmol, 17.5 mg/ml in ACN) and Cs₂CO₃ (57.3 mg, 0.176 mmol) in a 3:1 mixture of 1,2-dimethoxyethane (DME) (2 ml):water (0.7 ml) was added PdCl₂dppf (5.15 mg, 7.04 μmol). The mixture was flushed with nitrogen and heated in the microwave at 90° C. for 3 hours. The reaction was concentrated onto celite and purified by silica gel chromatography eluting with 0-10% MeOH/DCM+1% NH₄OH, followed by reverse phase chromatography (C18, 0-100% ACN/H₂O) to give the title compound (5.1 mg, 15.1% yield) as a yellow solid. ¹H NMR (500 MHz, DMSO-d₆) δ ppm 8.03 (br. s., 1H), 7.76 (d, J=8.19 Hz, 2H), 7.50 (s, 1H), 7.43 (d, J=11.98 Hz, 1H), 7.05 (d, J=8.68 Hz, 2H), 6.84 (br. s., 2H), 3.98 (d, J=11.49 Hz, 1H), 3.55-3.66 (m, 3H), 2.97 (t, J=5.87 Hz, 2H), 2.66-2.75 (m, 1H), 1.68-1.80 (m, 1H), 0.96 (dd, J=6.42, 3.36 Hz, 6H); LCMS: [M+H]⁺=480.35.

### Example 36: 6-(3-amino-5-fluoro-6-(4-morpholinophenyl)pyrazin-2-yl)-8-fluoroisoquinolin-1(2H)-one (I-36)

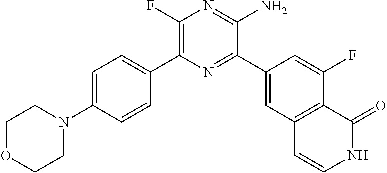 [SMILES: Nc1nc(F)c(-c2ccc(N3CCOCC3)cc2)nc1-c1cc(F)c2c(=O)[nH]ccc2c1]

Prepared from 6-(3-amino-6-bromo-5-fluoropyrazin-2-yl)-8-fluoroisoquinolin-1(2H)-one (35 mg, 0.099 mmol) and 4-(morpholino)phenylboronic acid (24.62 mg, 0.119 mmol) to give the title compound (11.3 mg, 26.2% yield) as a yellow solid. ¹H NMR (500 MHz, DMSO-d₆) δ ppm 11.27 (br. s., 1H), 7.75-7.84 (m, 3H), 7.48 (d, J=12.23 Hz, 1H), 7.22 (br. s., 1H), 7.04 (d, J=8.07 Hz, 2H), 6.87 (br. s., 2H), 6.62 (d, J=6.97 Hz, 1H), 3.75 (br. s., 4H), 3.17 (br. s., 4H); LCMS: [M+H]⁺=436.47.

### Example 37: 7-(3-amino-5-fluoro-6-(4-(4-isopropylpiperazin-1-yl)phenyl)pyrazin-2-yl)quinazolin-4(3H)-one (I-37) 21393

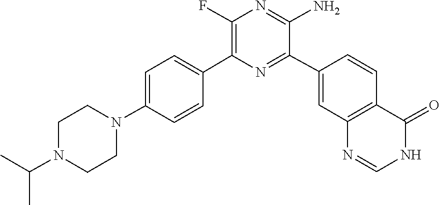 [SMILES: CC(C)N1CCN(c2ccc(-c3nc(-c4ccc5c(=O)[nH]cnc5c4)c(N)nc3F)cc2)CC1]

### Step 1: (4-oxo-3,4-dihydroquinazolin-7-yl)boronic acid

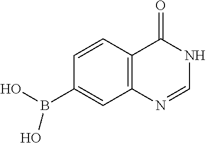 [SMILES: O=c1[nH]cnc2cc(B(O)O)ccc12]

To a round bottom flask charged with 7-bromoquinazolin-4(3h)-one (790 mg, 3.51 mmol), bis(pinacolato)diboron (981 mg, 3.86 mmol), potassium acetate (1034 mg, 10.53 mmol), was added anhydrous 1,4-dioxane (40 ml). The system was degassed then PdCl₂dppf (257 mg, 0.351 mmol) was added. The system was flushed with nitrogen then heated at 100° C. overnight on the stir plate. LCMS analysis showed a 66:34 mixture of the boronic acid and boronate. The reaction was diluted with acetonitrile, filtered through a pad of celite, concentrated in vacuo and used as-is the next step assuming full conversion to the boronic acid. LCMS boronate: [M+H]⁺=273.43, LCMS boronic acid: LCMS: [M+H]⁺=191.29.

### Step 2: 7-(3-amino-6-bromo-5-fluoropyrazin-2-yl)quinazolin-4(3H)

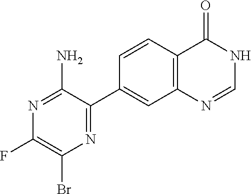 [SMILES: Nc1nc(F)c(Br)nc1-c1ccc2c(=O)[nH]cnc2c1]

To a solution of 5-bromo-6-fluoro-3-iodopyrazin-2-amine (100 mg, 0.283 mmol) and (4-oxo-3,4-dihydroquinazolin-7-yl)boronic acid (1.9 ml, 0.340 mmol, 33.3 mg/ml in ACN) in acetonitrile (4 ml), in a microwave vial, was added a solution of sodium carbonate monohydrate (73.7 mg, 0.595 mmol) in water (1 ml). The system was degassed and bis(triphenylphosphine)palladium(II) dichloride (23.85 mg, 0.034 mmol) was added. The reaction was heated in the microwave at 80° C. for 7 hours. The reaction was concentrated onto celite and purified by silica gel chromatography eluting with 0-10% MeOH/DCM+1% NH₄OH to give the product (92 mg, 97% yield) as a beige solid. LCMS: [M+H]+=336.31.

### Step 3: 7-(3-amino-5-fluoro-6-(4-(4-isopropylpiperazin-1-yl)phenyl)pyrazin-2-yl)quinazolin-4(3H)-one

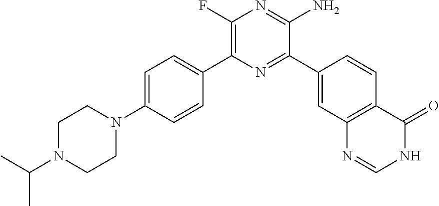 [SMILES: CC(C)N1CCN(c2ccc(-c3nc(-c4ccc5c(=O)[nH]cnc5c4)c(N)nc3F)cc2)CC1]

To a degassed suspension of 7-(3-amino-6-bromo-5-fluoropyrazin-2-yl)quinazolin-4(3H)-one (35 mg, 0.104 mmol), 4-(4-isopropylpiperazinyl)phenylboronic acid, pinacol ester (41.3 mg, 0.125 mmol) and Cs₂CO₃ (85 mg, 0.260 mmol) in a 3:1 mixture of 1,2-dimethoxyethane (DME) (4 ml):water (1.3 ml) was added PdCl₂dppf (7.62 mg, 10.41 μmol). The mixture was flushed with nitrogen and heated at 90° C. on the stir plate for 1 h. The reaction was concentrated onto celite and purified by silica gel chromatography eluting with 0-10% MeOH/DCM+1% NH₄OH, followed by reverse phase chromatography (C18, 0-100% ACN/H₂O). The desired fractions were then passed through an Isolute SCX-2 cation exchange resin cartridge eluting with 3% NH₃ in MeOH to give the title compound (8.2 mg, 17.1% yield) as a yellow solid. ¹H NMR (500 MHz, DMSO-d₆) δ ppm 12.24 (br. s., 1H), 8.16 (d, J=8.31 Hz, 1H), 8.09 (d, J=2.57 Hz, 1H), 7.92 (d, J=1.34 Hz, 1H), 7.80 (dd, J=8.31, 1.59 Hz, 1H), 7.70 (d, J=8.19 Hz, 2H), 6.97 (d, J=7.70 Hz, 2H), 6.75 (br. s., 2H), 3.04-3.19 (m, 4H), 2.82-2.93 (m, 1H), 2.49-2.66 (m, 4H), 0.99 (br. s., 6H); LCMS: [M+H]⁺=460.41.

### Example 38: 6-(3-amino-5-fluoro-6-(4-(4-isopropylpiperazin-1-yl)phenyl)pyrazin-2-yl)phthalazin-1(2H)-one (I-38) OICR21395A01

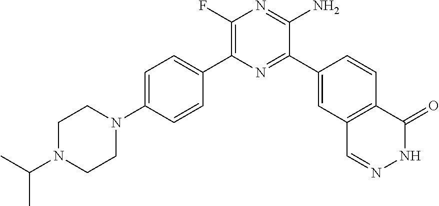 [SMILES: CC(C)N1CCN(c2ccc(-c3nc(-c4ccc5c(=O)[nH]ncc5c4)c(N)nc3F)cc2)CC1]

### Step 1: (1-oxo-1,2-dihydrophthalazin-6-yl)boronic acid

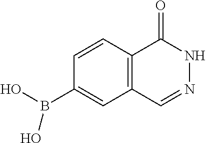 [SMILES: O=c1[nH]ncc2cc(B(O)O)ccc12]

To a round bottom flask charged with 6-bromophthalazin-1(2H)-one (500 mg, 2.222 mmol), bis(pinacolato)diboron (621 mg, 2.444 mmol), potassium acetate (654 mg, 6.67 mmol), was added anhydrous 1,4-dioxane (40 ml). The system was degassed then PdCl₂dppf (163 mg, 0.222 mmol) was added. The mixture was flushed with nitrogen then heated conventionally at 100° C. overnight. LCMS analysis showed complete conversion of the SM with a 92:8 mixture of the boronic acid and boronate. The reaction was diluted with acetonitrile, filtered through a pad of celite, concentrated in vacuo and used as-is the next step assuming full conversion to the boronic acid. LCMS Boronate: [M+H]+=273.30, LCMS Boronic acid: [M+H]+=191.23.

### Step 2: 6-(3-amino-6-bromo-5-fluoropyrazin-2-yl)phthalazin-1(2H)-one

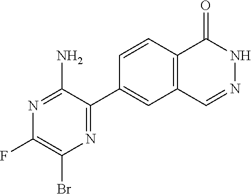 [SMILES: Nc1nc(F)c(Br)nc1-c1ccc2c(=O)[nH]ncc2c1]

To a solution of 5-bromo-6-fluoro-3-iodopyrazin-2-amine (100 mg, 0.283 mmol) and (1-oxo-1,2-dihydrophthalazin-6-yl)boronic acid (2.3 ml, 0.340 mmol, 28.1 mg/ml in ACN) in acetonitrile (4 ml) was added a solution of sodium carbonate monohydrate (73.7 mg, 0.595 mmol) in water (1 ml). The system was degassed and bis(triphenylphosphine)palladium(II) dichloride (23.85 mg, 0.034 mmol) was added. The reaction was flushed with nitrogen and heated on the stir plate at 80° C. overnight. The reaction was concentrated onto celite and purified by silica gel chromatography eluting with 0-100% EtOAc/Hexanes to give the product (0.125 mmol, 44.1% yield) as a beige solid. LCMS: [M+H]⁺=336.18.

### Step 3: 6-(3-amino-5-fluoro-6-(4-(4-isopropylpiperazin-1-yl)phenyl)pyrazin-2-yl)phthalazin-1(2H)-one

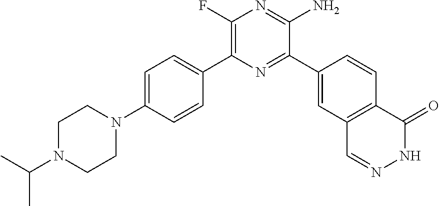 [SMILES: CC(C)N1CCN(c2ccc(-c3nc(-c4ccc5c(=O)[nH]ncc5c4)c(N)nc3F)cc2)CC1]

To a suspension of 6-(3-amino-6-bromo-5-fluoropyrazin-2-yl)phthalazin-1(2H)-one (35 mg, 0.104 mmol), 4-(4-isopropylpiperazinyl)phenylboronic acid, pinacol ester (41.3 mg, 0.125 mmol) and cesium carbonate (85 mg, 0.260 mmol) in a 3:1 mixture of 1,2-dimethoxyethane (DME) (4 ml):water (1.3 ml) was added PdCl₂dppf (7.62 mg, 10.41 μmol). The mixture was flushed with nitrogen and heated at 90° C. on the stir plate for 1 hour. The reaction was concentrated onto celite and purified by silica gel chromatography eluting with 0-10% MeOH/DCM+1% NH₄OH, followed by reverse phase chromatography (C18, 0-100% ACN/H₂O). The desired fractions were then passed through an Isolute SCX-2 cation exchange resin cartridge eluting with 3% NH₃ in MeOH to give the title compound (14.8 mg, 30.9% yield) as a yellow solid. ¹H NMR (500 MHz, DMSO-d₆) δ ppm 12.68 (s, 1H), 8.44 (s, 1H), 8.32 (d, J=8.31 Hz, 1H), 8.29 (s, 1H), 8.17 (dd, J=8.25, 1.53 Hz, 1H), 7.77 (d, J=8.07 Hz, 2H), 7.02 (d, J=8.80 Hz, 2H), 6.89 (s, 2H), 3.18 (m, J=8.70 Hz, 4H), 2.66-2.72 (m, 1H), 2.56-2.63 (m, 4H), 1.02 (br. s., 6H); LCMS: [M+H]⁺=460.41.

### Example 39: 7-(3-amino-5-fluoro-6-(4-(4-isopropylpiperazin-1-yl)phenyl)pyrazin-2-yl)-2-methylquinazolin-4(3H)-one (I-39)

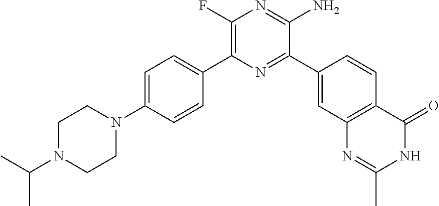 [SMILES: Cc1nc2cc(-c3nc(-c4ccc(N5CCN(C(C)C)CC5)cc4)c(F)nc3N)ccc2c(=O)[nH]1]

### Step 1: (2-methyl-4-oxo-3,4-dihydroquinazolin-7-yl)boronic acid

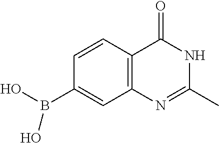

Prepared from 7-bromo-2-methylquinazolin-4(3H)-one (350 mg, 1.464 mmol) to give the boronic acid which was used in the next step without further purification. LCMS: [M+H]⁺ 205.34.

### Step 2: 7-(3-amino-5-fluoro-6-(4-(4-isopropylpiperazin-1-yl)phenyl)pyrazin-2-yl)-2-methylquinazolin-4(3H)-one

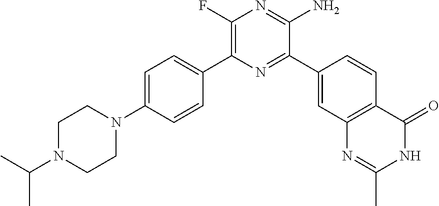 [SMILES: Cc1nc2cc(-c3nc(-c4ccc(N5CCN(C(C)C)CC5)cc4)c(F)nc3N)ccc2c(=O)[nH]1]

Prepared from 3-bromo-6-fluoro-5-(4-(4-isopropylpiperazin-1-yl)phenyl)pyrazin-2-amine (80 mg, 0.203 mmol) and (2-methyl-4-oxo-3,4-dihydroquinazolin-7-yl)boronic acid (53.8 mg, 0.264 mmol) to give the title compound (68.3 mg, 71.1% yield) as a yellow solid. ¹H NMR (500 MHz, DMSO-d₆) δ ppm 12.24 (br. s., 1H), 8.17 (d, J=8.19 Hz, 1H), 7.90 (d, J=1.22 Hz, 1H), 7.80 (dd, J=8.19, 1.47 Hz, 1H), 7.76 (d, J=7.95 Hz, 2H), 7.01 (d, J=8.93 Hz, 2H), 6.78 (s, 2H), 3.16-3.22 (m, 4H), 2.67 (dt, J=12.96, 6.48 Hz, 1H), 2.55-2.61 (m, 4H), 2.38 (s, 3H), 1.01 (d, J=6.48 Hz, 6H); LCMS: [M+H]⁺=474.51.

### Example 40: 7-(3-amino-5-fluoro-6-(4-(4-isopropylpiperazin-1-yl)phenyl)pyrazin-2-yl)-5-fluoro-2-methylquinazolin-4(3H)-one (I-40) Prophetic

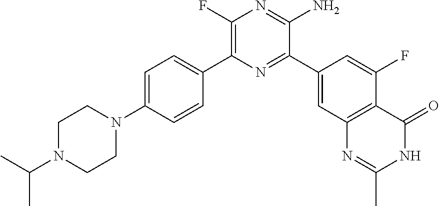 [SMILES: Cc1nc2cc(-c3nc(-c4ccc(N5CCN(C(C)C)CC5)cc4)c(F)nc3N)cc(F)c2c(=O)[nH]1]

1-40 can be prepared from processes similar to Example 2, Step 2 from 7-(3-amino-6-bromo-5-fluoropyrazin-2-yl)-5-fluoro-2-methylquinazolin-4(3H)-one and 1-isopropyl-4-(4-(4,4,5,5-tetramethyl-1,3,2-dioxaborolan-2-yl)phenyl)piperazine.

### Example 41: 6-(3-amino-5-fluoro-6-(4-(4-methylpiperazin-1-yl)phenyl)pyrazin-2-yl)-3,4-dihydroisoquinolin-1(2H)-one (I-41)

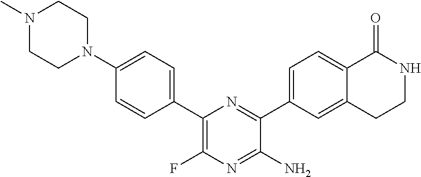 [SMILES: CN1CCN(c2ccc(-c3nc(-c4ccc5c(c4)CCNC5=O)c(N)nc3F)cc2)CC1]

In a microwave vial with magnetic stir bar was placed 6-(3-amino-6-bromo-5-fluoropyrazin-2-yl)-3,4-dihydroisoquinolin-1(2H)-one (84 mg, 0.249 mmol), 4-(4-methylpiperazin-1-yl)phenylboronic acid, pinacol ester (90 mg, 0.299 mmol), [1,12-Bis(diphenylphosphino)ferrocene]dichloropalladium(II) (18.23 mg, 0.025 mmol), Potassium phosphate tribasic reagent grade, m (0.383 mL, 0.498 mmol), DME (4 mL) and H₂O (2 mL) (4:1 DME/H₂O). The flask was sealed, then heated to 90° C. for 1 h in the microwave (high absorbance) when LCMS indicated complete conversion. The reaction was loaded onto Celite and purified by sgc (100% DCM to 95-5-1 DCM/MeOH/NH₄OH), followed by purification by reverse phase chromatography (C18, MeCN—H2O). Reverse phase fractions containing product were collected and poured into a column containing PoraPak RxnCX acidic PE resin. The resin was washed with MeOH, then the product was eluted off the column with 3% NH₄OH in MeOH to give the title compound as a yellow solid: (75 mg, 63.3% yield). ¹H NMR (500 MHz, DMSO-d₆) δ 7.98 (br s, 1H), 7.94 (d, J=7.9 Hz, 1H), 7.74 (d, J=7.9 Hz, 2H), 7.68 (dd, J=7.9, 1.3 Hz, 1H), 7.64 (s, 1H), 7.01 (d, J=8.9 Hz, 2H), 6.71 (s, 2H), 3.42 (td, J=6.5, 2.6 Hz, 2H), 3.22-3.18 (m, 4H), 2.98 (t, J=6.5 Hz, 2H), 2.48-2.43 (m, 4H), 2.22 (s, 3H); LCMS: [M+1]⁺=433.38.

### Example 42: 6-(3-amino-6-(3-((dimethylamino)methyl)-4-(tetrahydro-2H-pyran-4-yl)phenyl)-5-fluoropyrazin-2-yl)-4-fluoroisoquinolin-1(2H)-one (I-42)

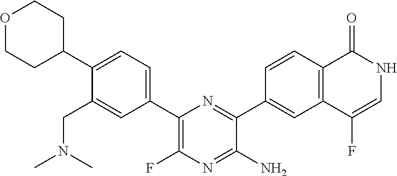 [SMILES: CN(C)Cc1cc(-c2nc(-c3ccc4c(=O)[nH]cc(F)c4c3)c(N)nc2F)ccc1C1CCOCC1]

### Step 1: 6-bromo-4-fluoroisoquinolin-1(2H)-one

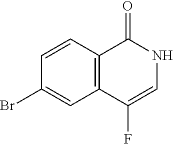 [SMILES: O=c1[nH]cc(F)c2cc(Br)ccc12]

A vial was charged with 6-bromo-2H-isoquinolin-1-one (1000 mg, 4.46 mmol) and Selectfluor™ fluorinating reagent >95% in F+ active (1897 mg, 5.36 mmol). Methanol (10 ml) and acetonitrile (10 ml) were added, and the reaction was heated to 50° C. for 1 h. The reaction was concentrated in vacuo, dissolved in 1,2-dichloroethane (20 ml), and phosphorous (V) oxychloride (0.835 ml, 8.93 mmol) was added. The reaction was stirred at 50° C. for 1 h. The mixture was poured into a RBF and concentrated in vacuo to remove the volatiles then the aqueous suspension was filtered, washing with water. The collected solid was dried under vacuum to give 6-bromo-4-fluoro-3-methoxy-3,4-dihydroisoquinolin-1(2H)-one (1.1 g, 102%) as a light pink solid. The material was used in the next step as-is. ¹H NMR (500 MHz, DMSO-d₆) δ ppm 11.29 (br. s., 1H), 8.12 (dd, J=8.56, 1.83 Hz, 1H), 7.91 (d, J=1.71 Hz, 1H), 7.78 (dd, J=8.56, 1.83 Hz, 1H), 7.47 (d, J=5.38 Hz, 1H); LCMS [M+H]⁺=242.14.

### Step 2: (4-fluoro-1-oxo-1,2-dihydroisoquinolin-6-yl)boronic acid

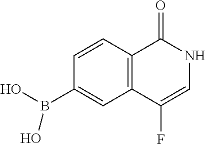 [SMILES: O=c1[nH]cc(F)c2cc(B(O)O)ccc12]

Prepared from 6-bromo-4-fluoroisoquinolin-1(2H)-one (920 mg, 3.80 mmol) to give a mixture of boronate and boronic acid which was used in the next step without further purification. LCMS: [M+H]+=290.31 Boronate, 208.21 Boronic acid.

### Step 3: 6-(3-amino-6-bromo-5-fluoropyrazin-2-yl)-4-fluoroisoquinolin-1(2H)-one

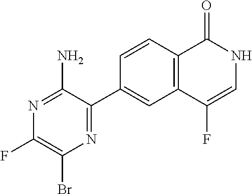 [SMILES: Nc1nc(F)c(Br)nc1-c1ccc2c(=O)[nH]cc(F)c2c1]

Prepared from 5-bromo-6-fluoro-3-iodopyrazin-2-amine (500 mg, 1.573 mmol) and (4-fluoro-1-oxo-1,2-dihydroisoquinolin-6-yl)boronic acid (391 mg, 1.887 mmol) to give 6-(3-amino-6-bromo-5-fluoropyrazin-2-yl)-4-fluoroisoquinolin-1(2H)-one (375 mg, 1.062 mmol, 67.5% yield) as a yellow solid. ¹H NMR (500 MHz, DMSO-d₆) δ ppm 11.23 (d, J=4.52 Hz, 1H), 8.30 (d, J=8.31 Hz, 1H), 7.96 (s, 1H), 7.82 (d, J=8.31 Hz, 1H), 7.45 (t, J=5.99 Hz, 1H), 7.13 (br. s., 2H); LCMS [M+H]⁺=353.19.

### Step 4: Methyl 2-(3,6-dihydro-2H-pyran-4-yl)-5-nitrobenzoate

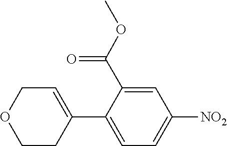 [SMILES: COC(=O)c1cc([N+](=O)[O-])ccc1C1=CCOCC1]

To a degassed solution of methyl 2-iodo-5-nitrobenzoate (4 g, 13.02 mmol), 2-(3,6-dihydro-2H-pyran-4-yl)-4,4,5,5-tetramethyl-1,3,2-dioxaborolane (3.4 g, 13.15 mmol) and K₂CO₃ (4.5 g, 32.56 mmol) in dioxane:water (7.5:2.5, 40 mL), Pd(dppf)Cl2·DCM (1.06 g, 1.3 mmol) was added and further degassed with N2 for 10 minutes and the reaction mass was stirred at 90° C. for 16 h. After completion of reaction, reaction mass was diluted with water (40 mL) and extracted with EtOAc (3×30 mL). The crude was purified by column chromatography and the product was eluted in 18% EtOAc in hexanes to afford the product (3.1 g, 90%) as off-white solid. ¹H NMR (400 MHz, Chloroform-d) δ 8.74 (d, J=2.4 Hz, 1H), 8.47 (s, 1H), 8.35 (dd, J=8.4, 2.5 Hz, 1H), 7.46 (d, J=8.4 Hz, 1H), 5.7 (s, 1H), 4.35 (q, J=2.7 Hz, 2H), 3.98 (d, J=6.6 Hz, 5H), 2.41 (tq, J=4.9, 2.3 Hz, 2H).

### Step 5: Methyl 5-amino-2-(tetrahydro-2H-pyran-4-yl)benzoate

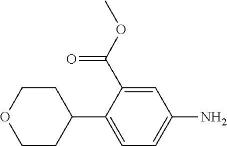 [SMILES: COC(=O)c1cc(N)ccc1C1CCOCC1]

In a 3 neck RBF, 10% Pd/C (50% moist, 2 w/w, 7.6 g) and methyl 2-(3,6-dihydro-2H-pyran-4-yl)-5-nitrobenzoate (3.8 g, 14.43 mmol) were added in EtOAc (40 mL). The reaction mass was stirred at room temperature with continuous purging of hydrogen for 2.5 days. After completion of reaction, reaction mass was filtered through celite bed and the filtrate was concentrated under vacuum to afford the crude product (3.2 g, 94%) as pale yellow gummy solid used as such in next step without further purification. LCMS: [M+H]⁺=236.5.

### Step 6: Methyl 5-bromo-2-(tetrahydro-2H-pyran-4-yl)benzoate

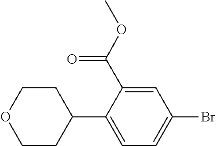 [SMILES: COC(=O)c1cc(Br)ccc1C1CCOCC1]

To a solution of methyl 5-amino-2-(tetrahydro-2H-pyran-4-yl)benzoate (3 g, 12.74 mmol) in bromoform (9.6 mL). To this, tert-butylnitrite (15 mL) was added dropwise at room temperature and the reaction mass was stirred at same temperature for 30 minutes. After completion of reaction, the reaction mass was concentrated to afford crude. The crude was purified by column chromatography and the product was eluted in 10% EtOAc in hexanes to afford the product (1.8 g, 47%) as a yellow solid. ¹H NMR (400 MHz, Chloroform-d) 1.76-1.90 (m, 4H), 3.63 (m, 4H), 3.95 (s, 3H), 4.11 (dt, J=11.1, 2.9 Hz, 1H), 7.32 (d, J=8.4 Hz, 1H), 7.64 (dd, J=8.4, 2.3 Hz, 1H), 7.98 (d, J=2.4 Hz, 1H).

### Step 7: (5-bromo-2-(tetrahydro-2H-pyran-4-yl)phenyl)methanol

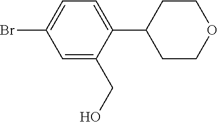 [SMILES: OCc1cc(Br)ccc1C1CCOCC1]

To a cooled solution of methyl 5-bromo-2-(tetrahydro-2H-pyran-4-yl)benzoate (1.8 g, 6.02 mmol) in dry THF (18 mL) at 0° C., LAH (1 M in THF, 6.12 mL, 6.02 mmol) was added dropwise at 0° C. The reaction mass was stirred at RT for 3 h. After completion of reaction, reaction mass was quenched with the dil. HCl (50 mL) and extracted with ethyl acetate (3×50 mL). The combined organic was dried over anhydrous Na₂SO₄, concentrate under vacuum to afford the title compound (1.3 g, 4.79 mmol, 79.68%) as pale-yellow liquid. ¹H NMR (400 MHz, Chloroform-d) 1.67-1.90 (m, 4H), 3.05 (m, 1H), 3.58 (m, 2H), 4.10 (d, J=11.1, 1H), 4.73 (s, 2H), 7.21 (d, J=8.4 Hz, 1H), 7.47 (dd, J=8.4, 2.3 Hz, 1H), 7.55 (d, J=2.4 Hz, 1H).

### Step 8: 5-bromo-2-(tetrahydro-2H-pyran-4-yl)benzaldehyde

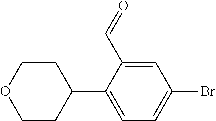 [SMILES: O=Cc1cc(Br)ccc1C1CCOCC1]

To a solution of (5-bromo-2-(tetrahydro-2H-pyran-4-yl)phenyl)methanol (1.3 g, 4.79 mmol) in DCM (26 mL), MnO₂ (3.3 g, 38.35 mmol) was added at room temperature and the reaction mass was stirred at room temperature for 48 h. After completion of reaction, reaction mass was filtered through celite bed and the filtrate was concentrate under vacuum to afford crude product. The crude was purified by column chromatography and the product was eluted in 15% EtOAc in hexanes to afford the product (0.76 g, 58.9%) as light-yellow solid. ¹H NMR (400 MHz, Chloroform-d) δ 1.76-1.84 (m, 2H), 1.86-1.93 (m, J=12.2, 4.3 Hz, 2H), 3.61-3.67 (t, J=11.7 Hz, 2H), 3.89-3.83 (tt, J=3.2, 4.0 Hz, 1H), 4.15-4.12 (dd, J=11.4, 4.6 Hz, 2H), 7.39-7.37 (t, J=8.4 Hz, 1H), 7.77 (d, 1H), 7.98 (s, 1H), 10.27 (s, 1H).

### Step 9: 1-(5-bromo-2-(tetrahydro-2H-pyran-4-yl)phenyl)-N,N-dimethylmethanamine

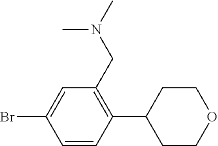 [SMILES: CN(C)Cc1cc(Br)ccc1C1CCOCC1]

Prepared from 5-bromo-2-(tetrahydro-2H-pyran-4-yl)benzaldehyde (400 mg, 1.486 mmol) and dimethylamine, 2.0M solution in THF (2.229 ml, 4.46 mmol) to obtain 1-(5-bromo-2-(tetrahydro-2H-pyran-4-yl)phenyl)-N,N-dimethylmethanamine (394 mg, 1.321 mmol, 89% yield) as a white solid. The material was carried onto the next step without further purification. LCMS: [M+H]⁺=298.34.

### Step 10: N,N-dimethyl-1-(2-(tetrahydro-2H-pyran-4-yl)-5-(4,4,5,5-tetramethyl-1,3,2-dioxaborolan-2-yl)phenyl)methanamine

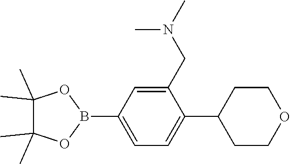 [SMILES: CN(C)Cc1cc(B2OC(C)(C)C(C)(C)O2)ccc1C1CCOCC1]

Prepared from 1-(5-bromo-2-(tetrahydro-2H-pyran-4-yl)phenyl)-N,N-dimethylmethanamine (385 mg, 1.291 mmol) to give the boronate which was used in the next step without further purification. LCMS: [M+H]⁺=346.55.

### Step 11: 6-(3-amino-6-(3-((dimethylamino)methyl)-4-(tetrahydro-2H-pyran-4-yl)phenyl)-5-fluoropyrazin-2-yl)-4-fluoroisoquinolin-1(2H)-one

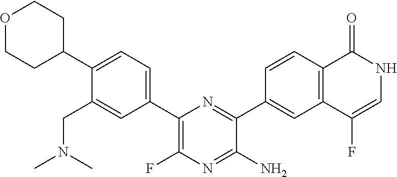 [SMILES: CN(C)Cc1cc(-c2nc(-c3ccc4c(=O)[nH]cc(F)c4c3)c(N)nc2F)ccc1C1CCOCC1]

Prepared from 6-(3-amino-6-bromo-5-fluoropyrazin-2-yl)-4-fluoroisoquinolin-1(2H)-one (40 mg, 0.113 mmol) and N,N-dimethyl-1-(2-(tetrahydro-2H-pyran-4-yl)-5-(4,4,5,5-tetramethyl-1,3,2-dioxaborolan-2-yl)phenyl)methanamine (46.9 mg, 0.136 mmol) to give the title compound (30.1 mg, 54% yield) as a beige solid. ¹H NMR (500 MHz, DMSO-d₆) δ ppm 11.22 (br. s., 1H), 8.32 (dd, J=8.31, 1.71 Hz, 1H), 8.05 (d, J=1.22 Hz, 1H), 7.94 (dd, J=8.31, 1.47 Hz, 1H), 7.75 (d, J=8.31 Hz, 1H), 7.73 (s, 1H), 7.45 (t, J=5.14 Hz, 1H), 7.41 (d, J=8.19 Hz, 1H), 7.00 (s, 2H), 3.96 (dd, J=10.94, 3.48 Hz, 2H), 3.42-3.47 (m, 4H), 3.18-3.24 (m, 1H), 2.17 (s, 6H), 1.72 (qd, J=12.27, 4.03 Hz, 2H), 1.59-1.66 (m, 2H); LCMS [M+H]⁺=492.40.

### Example 43: 6-(3-amino-5-fluoro-6-(4-(4-isopropylpiperazin-1-yl)phenyl)pyrazin-2-yl)-4-chloro-3-methylisoquinolin-1(2H)-one (I-43)

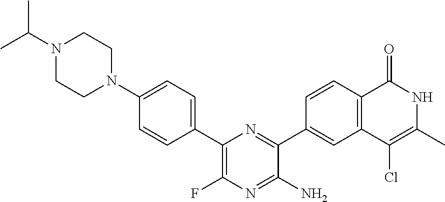 [SMILES: Cc1[nH]c(=O)c2ccc(-c3nc(-c4ccc(N5CCN(C(C)C)CC5)cc4)c(F)nc3N)cc2c1Cl]

### Step 1: 6-bromo-4-chloro-3-methylisoquinolin-1(2H)-one

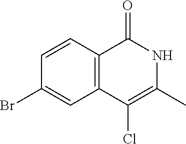

To a solution of 6-bromo-3-methyl-2H-isoquinolin-1-one (1.0 g, 4.20 mmol) in N,N-dimethylacetamide (14 ml) was added N-chlorosuccinimide (0.673 g, 5.04 mmol). The mixture was stirred at 50° C. for 8 hours. The reaction was cooled to RT and diluted with water. The suspension was filtered and washed with water. The solid was further triturated from MeOH and dried under vacuum to obtain 6-bromo-4-chloro-3-methylisoquinolin-1(2H)-one (1.12 g, 4.11 mmol, 98% yield) as a white solid. LCMS: [M+H]⁺=272.20.

### Step 2: (4-chloro-3-methyl-1-oxo-1,2-dihydroisoquinolin-6-yl)boronic acid

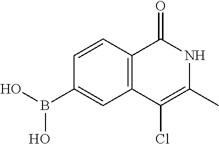 [SMILES: Cc1[nH]c(=O)c2ccc(B(O)O)cc2c1Cl]

Prepared from 6-bromo-4-chloro-3-methylisoquinolin-1(2H)-one (1.1 g, 4.04 mmol) to give a mixture of boronate and boronic acid which was used in the next step without further purification. LCMS[M+H]⁺=238.34 Boronic acid, 320.34 Boronate.

### Step 3: 6-(3-amino-6-bromo-5-fluoropyrazin-2-yl)-4-chloro-3-methylisoquinolin-1(2H)-one

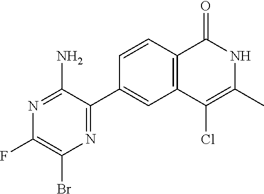 [SMILES: Cc1[nH]c(=O)c2ccc(-c3nc(Br)c(F)nc3N)cc2c1Cl]

Prepared from 5-bromo-6-fluoro-3-iodopyrazin-2-amine (425 mg, 1.337 mmol) and (4-chloro-3-methyl-1-oxo-1,2-dihydroisoquinolin-6-yl)boronic acid (444 mg, 1.872 mmol) to give the product (124 mg, 24% yield) as a beige solid. LCMS: [M+H]⁺=383.22.

### Step 4: 6-(3-amino-5-fluoro-6-(4-(4-isopropylpiperazin-1-yl)phenyl)pyrazin-2-yl)-4-chloro-3-methylisoquinolin-1(2H)-one

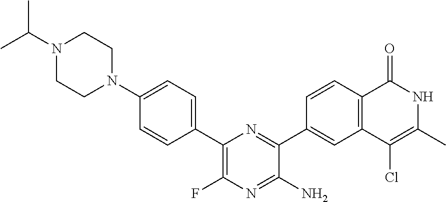 [SMILES: Cc1[nH]c(=O)c2ccc(-c3nc(-c4ccc(N5CCN(C(C)C)CC5)cc4)c(F)nc3N)cc2c1Cl]

Prepared from 6-(3-amino-6-bromo-5-fluoropyrazin-2-yl)-4-chloro-3-methylisoquinolin-1(2H)-one (27 mg, 0.070 mmol) and 4-(4-isopropylpiperazinyl)phenylboronic acid, pinacol ester (27.9 mg, 0.084 mmol) to give the title compound (19.1 mg, 54% yield) as a yellow solid. ¹H NMR (500 MHz, DMSO-d₆) δ ppm 11.71 (br. s., 1H), 8.30 (d, J=8.31 Hz, 1H), 8.16 (s, 1H), 7.87 (dd, J=8.31, 1.34 Hz, 1H), 7.75 (d, J=7.95 Hz, 2H), 7.02 (d, J=9.05 Hz, 2H), 6.81 (s, 2H), 3.17-3.21 (m, 4H), 2.65-2.71 (m, 1H), 2.56-2.60 (m, 4H), 2.39 (s, 3H), 1.01 (d, J=6.60 Hz, 6H); LCMS: [M+H]⁺=507.54.

### Example 44: 6-(3-amino-5-fluoro-6-(4-(4-(oxetan-3-yl)piperazin-1-yl)phenyl)pyrazin-2-yl)-8-fluoro-3,4-dihydroisoquinolin-1(2H)-one (I-44)

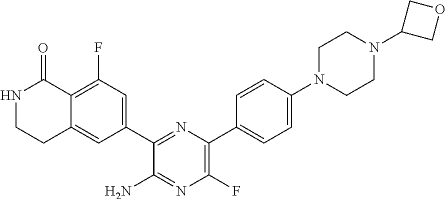 [SMILES: Nc1nc(F)c(-c2ccc(N3CCN(C4COC4)CC3)cc2)nc1-c1cc(F)c2c(c1)CCNC2=O]

Prepared from 6-(3-amino-6-bromo-5-fluoropyrazin-2-yl)-8-fluoro-3,4-dihydroisoquinolin-1(2H)-one (42.5 mg, 0.12 mmol) and 1-(oxetan-3-yl)-4-(4-(4,4,5,5-tetramethyl-1,3,2-dioxaborolan-2-yl)phenyl)piperazine to give the title compound (19.9 mg, 40% yield). ¹H NMR (500 MHz, DMSO-d₆) 5=8.03 (br s, 1H), 7.75 (d, J=7.8 Hz, 2H), 7.49 (s, 1H), 7.42 (d, J=12.0 Hz, 1H), 7.03 (d, J=8.9 Hz, 2H), 6.82 (s, 2H), 4.61-4.53 (m, 2H), 4.48 (t, J=6.1 Hz, 2H), 3.45 (quin, J=6.3 Hz, 1H), 3.39-3.34 (m, 2H), 3.27-3.20 (m, 4H), 2.97 (t, J=6.4 Hz, 2H), 2.44-2.39 (m, 4H); LCMS: [M+H]⁺=493.43.

### Example 45: 6-(3-amino-5-fluoro-6-(4-(4-(oxetan-3-yl)piperazin-1-yl)phenyl)pyrazin-2-yl)-7-fluoro-3,4-dihydroisoquinolin-1(2H)-one (I-45)

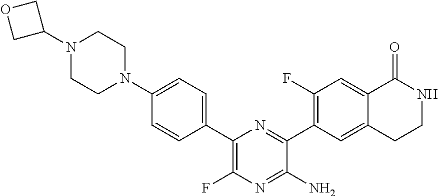 [SMILES: Nc1nc(F)c(-c2ccc(N3CCN(C4COC4)CC3)cc2)nc1-c1cc2c(cc1F)C(=O)NCC2]

Prepared from 6-(3-amino-6-chloro-5-fluoropyrazin-2-yl)-7-fluoro-3,4-dihydroisoquinolin-1(2H)-one (37.2 mg, 0.12 mmol) and 1-(oxetan-3-yl)-4-(4-(4,4,5,5-tetramethyl-1,3,2-dioxaborolan-2-yl)phenyl)piperazine (34.4 mg, 0.1 mmol) to give the title compound (19.6 mg, 37.1% yield). ¹H NMR (500 MHz, DMSO-d₆) 5=8.15 (br s, 1H), 7.70 (br d, J=8.1 Hz, 2H), 7.63 (d, J=10.1 Hz, 1H), 7.51 (br d, J=6.7 Hz, 1H), 7.01 (br d, J=8.8 Hz, 2H), 6.68 (s, 2H), 4.57 (t, J=6.4 Hz, 2H), 4.47 (t, J=6.0 Hz, 2H), 3.50-3.38 (m, 3H), 3.28-3.18 (m, 4H), 2.95 (br t, J=6.4 Hz, 2H), 2.41 (br d, J=4.2 Hz, 4H); LCMS: [M+H]⁺=493.57.

### Example 46: 6-(3-amino-5-fluoro-6-(4-(4-(oxetan-3-yl)piperazin-1-yl)phenyl)pyrazin-2-yl)-3,4-dihydroisoquinolin-1(2H)-one (I-46)

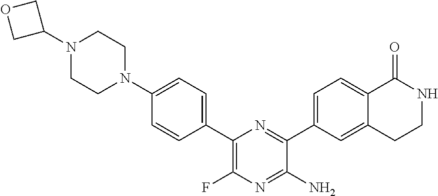 [SMILES: Nc1nc(F)c(-c2ccc(N3CCN(C4COC4)CC3)cc2)nc1-c1ccc2c(c1)CCNC2=O]

Prepared from 6-(3-amino-6-bromo-5-fluoropyrazin-2-yl)-3,4-dihydroisoquinolin-1(2H)-one (40.4 mg, 0.12 mmol) and 1-(oxetan-3-yl)-4-(4-(4,4,5,5-tetramethyl-1,3,2-dioxaborolan-2-yl)phenyl)piperazine (34.4 mg, 0.1 mmol) to give the title compound (22 mg, 46.2% yield). ¹H NMR (500 MHz, DMSO-d6) 5=7.97 (br s, 1H), 7.94 (d, J=8.1 Hz, 1H), 7.75 (d, J=7.9 Hz, 2H), 7.70-7.67 (m, 1H), 7.64 (s, 1H), 7.02 (d, J=8.9 Hz, 2H), 6.72 (s, 2H), 4.57 (t, J=6.5 Hz, 2H), 4.47 (t, J=6.0 Hz, 2H), 3.48-3.37 (m, 3H), 3.26-3.17 (m, 4H), 2.98 (br t, J=6.5 Hz, 2H), 2.46-2.39 (m, 4H); LCMS: [M+H]⁺=475.45.

### Example 47: 6-(3-amino-6-(4-(4-cyclobutylpiperazin-1-yl)phenyl)-5-fluoropyrazin-2-yl)-8-fluoro-3,4-dihydroisoquinolin-1(2H)-one (I-47)

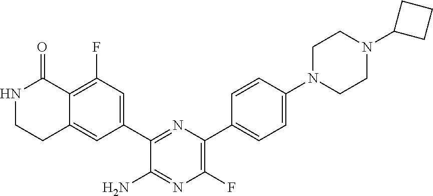 [SMILES: Nc1nc(F)c(-c2ccc(N3CCN(C4CCC4)CC3)cc2)nc1-c1cc(F)c2c(c1)CCNC2=O]

Prepared from 6-(3-amino-6-bromo-5-fluoropyrazin-2-yl)-8-fluoro-3,4-dihydroisoquinolin-1(2H)-one (42.6 mg, 0.12 mmol) and 1-cyclobutyl-4-(4-(4,4,5,5-tetramethyl-1,3,2-dioxaborolan-2-yl)phenyl)piperazine (34.2 mg, 0.1 mmol) to give the title compound (20.4 mg, 41.0% yield). ¹H NMR (500 MHz, DMSO-d₆) 5=8.03 (br s, 1H), 7.74 (d, J=7.8 Hz, 2H), 7.49 (s, 1H), 7.42 (d, J=11.9 Hz, 1H), 7.01 (d, J=9.0 Hz, 2H), 6.82 (s, 2H), 3.40-3.26 (m, 2H), 3.25-3.14 (m, 4H), 2.97 (br t, J=6.3 Hz, 2H), 2.73 (quin, J=7.7 Hz, 1H), 2.45-2.32 (m, 4H), 1.98 (td, J=3.9, 10.9 Hz, 2H), 1.88-1.76 (m, 2H), 1.71-1.59 (m, 2H); LCMS: [M+H]⁺=491.44.

### Example 48: 6-(3-amino-6-(4-(4-cyclobutylpiperazin-1-yl)phenyl)-5-fluoropyrazin-2-yl)-7-fluoro-3,4-dihydroisoquinolin-1(2H)-one (I-48)

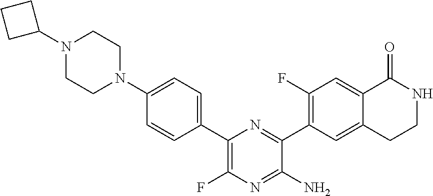 [SMILES: Nc1nc(F)c(-c2ccc(N3CCN(C4CCC4)CC3)cc2)nc1-c1cc2c(cc1F)C(=O)NCC2]

Prepared from 6-(3-amino-6-chloro-5-fluoropyrazin-2-yl)-7-fluoro-3,4-dihydroisoquinolin-1(2H)-one (37.3 mg, 0.12 mmol) reacted with 1-cyclobutyl-4-(4-(4,4,5,5-tetramethyl-1,3,2-dioxaborolan-2-yl)phenyl)piperazine (34.2 mg, 0.1 mmol) to give the title compound (19.1 mg, 38.2% yield). ¹H NMR (500 MHz, DMSO-d₆) δ=8.14 (br s, 1H), 7.69 (d, J=7.8 Hz, 2H), 7.63 (d, J=10.1 Hz, 1H), 7.51 (d, J=6.8 Hz, 1H), 6.99 (d, J=9.0 Hz, 2H), 6.67 (s, 2H), 3.42 (dt, J=2.8, 6.6 Hz, 2H), 3.21-3.13 (m, 4H), 2.95 (br t, J=6.4 Hz, 2H), 2.73 (quin, J=7.8 Hz, 1H), 2.41-2.34 (m, 4H), 2.04-1.94 (m, 2H), 1.88-1.75 (m, 2H), 1.71-1.59 (m, 2H); LCMS: [M+H]⁺=491.56;

### Example 49: 6-(3-amino-6-(4-(4-cyclobutylpiperazin-1-yl)phenyl)-5-fluoropyrazin-2-yl)-3,4-dihydroisoquinolin-1(2H)-one (I-49)

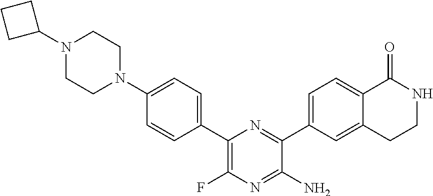 [SMILES: Nc1nc(F)c(-c2ccc(N3CCN(C4CCC4)CC3)cc2)nc1-c1ccc2c(c1)CCNC2=O]

Prepared from 6-(3-amino-6-bromo-5-fluoropyrazin-2-yl)-3,4-dihydroisoquinolin-1(2H)-one (40.4 mg, 0.12 mmol) and 1-cyclobutyl-4-(4-(4,4,5,5-tetramethyl-1,3,2-dioxaborolan-2-yl)phenyl)piperazine (34.2 mg, 0.1 mmol) to give the title compound (26.1 mg, 54.7% yield). ¹H NMR (500 MHz, DMSO-d₆) δ=7.98 (br s, 1H), 7.94 (d, J=7.9 Hz, 1H), 7.74 (br d, J=8.1 Hz, 2H), 7.68 (br d, J=7.9 Hz, 1H), 7.66-7.61 (m, 1H), 7.01 (br d, J=8.9 Hz, 2H), 6.72 (s, 2H), 3.46-3.39 (m, 2H), 3.24-3.14 (m, 4H), 2.98 (br t, J=6.3 Hz, 2H), 2.73 (quin, J=7.7 Hz, 1H), 2.43-2.33 (m, 4H), 2.04-1.92 (m, 2H), 1.87-1.77 (m, 2H), 1.72-1.60 (m, 2H); LCMS: [M+H]⁺=473.51.

### Example 50: (R)-6-(3-amino-5-fluoro-6-(4-(4-isopropyl-2-methylpiperazin-1-yl)phenyl)pyrazin-2-yl)-3,4-dihydroisoquinolin-1(2H)-one (I-50)

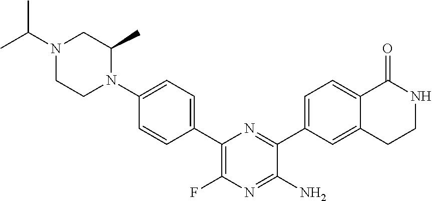 [SMILES: CC(C)N1CCN(c2ccc(-c3nc(-c4ccc5c(c4)CCNC5=O)c(N)nc3F)cc2)[C@H](C)C1]

### Step 1: tert-butyl (R)-4-isopropyl-2-methylpiperazine-1-carboxylate

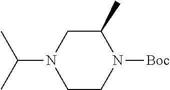 [SMILES: CC(C)N1CCN(C(=O)OC(C)(C)C)[C@H](C)C1]

To a solution of tert-butyl (R)-2-methylpiperazine-1-carboxylate (5 g, 24.96 mmol) in DMF (125 mL), sodium triacetoxyborohydride (10.58 g, 49.92 mmol) and acetone (2.89 g, 49.92 mmol), acetic acid (2.24 g, 37.44 mmol) were added at room temperature and the reaction mass was stirred at 60° C. for 3 h. After completion of the reaction, the mixture was diluted with cold saturated NaHCO₃ solution (150 mL) and extracted with EtOAc (3×60 mL). The aqueous layer was discarded and combined organic layer was washed with saturated citric acid solution (2×60 mL). The aqueous citric acid layer was made basic (pH=9) using NaHCO₃ and extracted with EtOAc (3×60 mL). The combined organic layer was dried over Na₂SO₄ and concentrated under vacuum to afford the title compound (5.4 g, 89% yield) as colourless liquid. LCMS: [M+H]⁺=243.3.

### Step 2: (R)-1-isopropyl-3-methylpiperazine

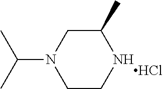

To a stirred solution of tert-butyl (R)-4-isopropyl-2-methylpiperazine-1-carboxylate (5.4 g, 22.28 mmol) in DCM (54 mL) at 0° C. 4N HCl in dioxane (54 mL) was added dropwise and the reaction mass was stirred at RT for 16 h. After completion of the reaction, the mixture was concentrated under vacuum to afford the title compound (5 g, quant. crude yield) as off-white gummy solid. LCMS: [M+H]⁺=143.1.

### Step 3: (R)-1-(4-bromophenyl)-4-isopropyl-2-methylpiperazine

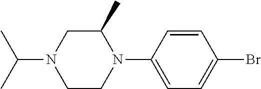 [SMILES: CC(C)N1CCN(c2ccc(Br)cc2)[C@H](C)C1]

To a stirred solution of (R)-1-isopropyl-3-methylpiperazine (2 g, 14.06 mmol) in toluene (30 mL), 1-bromo-4-iodobenzene (4.76 g, 16.87 mmol), palladium acetate (0.16 g, 0.703 mmol), tri-tert-butyl phophonium HBF₄ (0.44 g, 1.406 mmol), potassium tert-butoxide (5.04 g, 49.22 mmol) were added and the mixture was degassed using Ar for 10 min and stirred at 120° C. for 16 h. The mixture was poured into water (80 mL) and extracted with EtOAc (3×40 mL), dried over Na₂SO₄ and concentrated under vacuum to get crude compound. The crude was purified by column chromatography and product was eluted in 1.8% MeOH in DCM to afford the title compound (0.88 g, 21%) as brown. LCMS: [M+H]⁺=299.4.

### Step 4: (R)-6-(3-amino-5-fluoro-6-(4-(4-isopropyl-2-methylpiperazin-1-yl)phenyl)pyrazin-2-yl)-3,4-dihydroisoquinolin-1(2H)-one

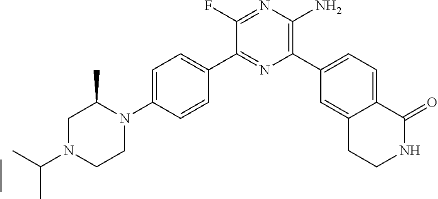

Prepared from 6-(3-amino-6-bromo-5-fluoropyrazin-2-yl)-3,4-dihydroisoquinolin-1(2H)-one (101 mg, 0.300 mmol) and (R)-4-isopropyl-2-methyl-1-(4-(4,4,5,5-tetramethyl-1,3,2-dioxaborolan-2-yl)phenyl)piperazine (124 mg, 0.36 mmol) give the title compound as a yellow solid: (50 mg, 41.0% yield). ¹H NMR (500 MHz, DMSO-d₆) δ 7.98 (br s, 1H), 7.94 (d, J=7.9 Hz, 1H), 7.74 (d, J=7.9 Hz, 2H), 7.69 (dd, J=7.9, 1.3 Hz, 1H), 7.65 (s, 1H), 6.94 (d, J=9.0 Hz, 2H), 6.69 (s, 2H), 4.06 (br dd, J=6.2, 3.1 Hz, 1H), 3.42 (td, J=6.5, 2.7 Hz, 2H), 3.39-3.34 (m, 1H), 2.98 (br t, J=6.5 Hz, 2H), 2.96-2.91 (m, 1H), 2.83 (br dd, J=10.6, 1.7 Hz, 1H), 2.70-2.62 (m, 2H), 2.43 (dd, J=10.9, 3.4 Hz, 1H), 2.30 (td, J=11.1, 3.3 Hz, 1H), 1.03 (d, J=6.5 Hz, 3H), 0.99 (t, J=7.0 Hz, 6H); LCMS: [M+1]⁺=475.07.

### Example 51: (R)-6-(3-amino-5-fluoro-6-(4-(4-isopropyl-2-methylpiperazin-1-yl)phenyl)pyrazin-2-yl)-7-fluoro-3,4-dihydroisoquinolin-1(2H)-one (I-51)

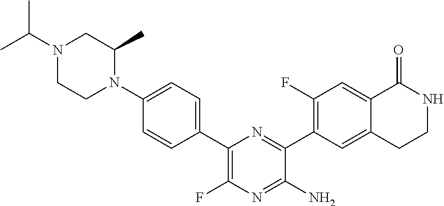 [SMILES: CC(C)N1CCN(c2ccc(-c3nc(-c4cc5c(cc4F)C(=O)NCC5)c(N)nc3F)cc2)[C@H](C)C1]

Prepared from 6-(3-amino-6-bromo-5-fluoropyrazin-2-yl)-7-fluoro-3,4-dihydroisoquinolin-1(2H)-one (107 mg, 0.300 mmol), (R)-4-isopropyl-2-methyl-1-(4-(4,4,5,5-tetramethyl-1,3,2-dioxaborolan-2-yl)phenyl)piperazine (124 mg, 0.36 mmol), to give the title compound as a yellow solid (63 mg, 41% yield). ¹H NMR (500 MHz, DMSO-d₆) δ 8.15 (br s, 1H), 7.69 (d, J=7.8 Hz, 2H), 7.64 (d, J=10.1 Hz, 1H), 7.51 (d, J=6.8 Hz, 1H), 6.93 (d, J=9.2 Hz, 2H), 6.65 (s, 2H), 4.06 (dt, J=6.2, 2.9 Hz, 1H), 3.42 (td, J=6.5, 2.8 Hz, 2H), 3.38-3.34 (m, 1H), 2.99-2.89 (m, 3H), 2.83 (br dd, J=10.6, 1.9 Hz, 1H), 2.71-2.61 (m, 2H), 2.43 (dd, J=10.9, 3.4 Hz, 1H), 2.29 (td, J=11.1, 3.3 Hz, 1H), 1.02 (d, J=6.4 Hz, 3H), 0.99 (t, J=7.1 Hz, 6H); LCMS: [M+1]⁺=493.13

### Example 52: (R)-6-(3-amino-6-(4-(2,4-dimethylpiperazin-1-yl)phenyl)-5-fluoropyrazin-2-yl)-3,4-dihydroisoquinolin-1(2H)-one (I-52)

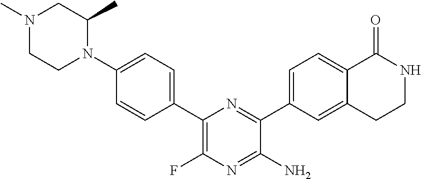 [SMILES: C[C@@H]1CN(C)CCN1c1ccc(-c2nc(-c3ccc4c(c3)CCNC4=O)c(N)nc2F)cc1]

### Step 1: tert-butyl (R)-2,4-dimethylpiperazine-1-carboxylate

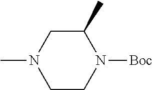

To a solution of tert-butyl (R)-2-methylpiperazine-1-carboxylate (5 g, 24.96 mmol) in DMF (125 mL), sodium triacetoxyborohydride (10.58 g, 49.92 mmol) and 37% formaldehyde (2.25 g, 74.88 mmol), acetic acid (2.24 g, 37.44 mmol) were added at RT and the reaction mass was stirred at 60° C. for 3 h. The mixture was diluted with cold saturated NaHCO₃ solution (150 mL) and extracted with EtOAc (3×60 mL). The aqueous layer was discarded and combined organic layer was washed with saturated citric acid solution (2×60 mL). The aqueous citric acid layer was made basic (pH=9) using NaHCO₃ and extracted with EtOAc (3×60 mL). The combined organic layer was dried over Na₂SO₄ and concentrated under vacuum to afford the product (3.2 g, 59.81% yield) as colourless. LCMS: [M+H]⁺=215.0.

### Step 2: (R)-1,3-dimethylpiperazine

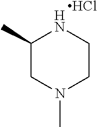

To a stirred solution of tert-butyl (R)-2,4-dimethylpiperazine-1-carboxylate (3.2 g, 14.93 mmol) in DCM (32 mL) at 0° C. 4N HCl in dioxane (32 mL) was added dropwise and the reaction mass was stirred at RT for 16 h. After completion of the reaction, the mixture was concentrated under vacuum to afford the title compound (3 g, quant. crude yield) as off-white gummy solid of (R)-1,3-dimethylpiperazine. ¹H NMR (400 MHz, DMSO-d₆) δ 3.75-3.53 (m, 4H), 3.36-3.25 (m, 2H), 3.15 (m, 1H), 2.81 (s, 3H), 1.32 (d, J=6.4 Hz, 3H).

### Step 3: (R)-1-(4-bromophenyl)-2,4-dimethylpiperazine

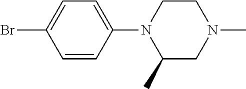

To a stirred solution of (R)-1,3-dimethylpiperazine (0.7 g, 6.12 mmol) in toluene (105 mL), 1-bromo-4-iodobenzene (1.96 g, 7.34 mmol), palladium acetate (0.07 g, 0.306 mmol), tri-tert-butyl phophonium HBF₄ (0.175 g, 0.612 mmol), potassium tert-butoxide (2.38 g, 21.43 mmol) were added and the reaction mass degassed using Ar for 10 min and stirred at 120° C. for 16 h. After completion of reaction, reaction mass was poured into water (30 mL) and extracted with EtOAc (3×15 mL), dried over anhydrous Na₂SO₄ and concentrated under vacuum to get crude compound which was purified by sgc, eluting with 1.6% MeOH in DCM, to afford the product (0.26 g, 15.8% yield) as brown solid. LCMS: [M+H]⁺=271.3.

### Step 4: (R)-2,4-dimethyl-1-(4-(4,4,5,5-tetramethyl-1,3,2-dioxaborolan-2-yl)phenyl)piperazine

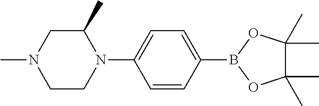

Prepared from (R)-1-(4-bromophenyl)-2,4-dimethylpiperazine (291 mg, 1.081 mmol) to give the product (342 mg, 100% yield).

### Step 5: (R)-6-(3-amino-6-(4-(2,4-dimethylpiperazin-1-yl)phenyl)-5-fluoropyrazin-2-yl)-3,4-dihydroisoquinolin-1(2H)-one

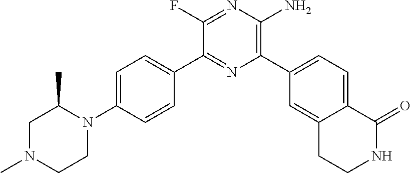 [SMILES: C[C@@H]1CN(C)CCN1c1ccc(-c2nc(-c3ccc4c(c3)CCNC4=O)c(N)nc2F)cc1]

Prepared from 6-(3-amino-6-bromo-5-fluoropyrazin-2-yl)-3,4-dihydroisoquinolin-1(2H)-one (101 mg, 0.300 mmol), (R)-2,4-dimethyl-1-(4-(4,4,5,5-tetramethyl-1,3,2-dioxaborolan-2-yl)phenyl)piperazine (114 mg, 0.36 mmol) to give the title compound as a yellow solid, (50 mg, 37.0% yield). ¹H NMR (500 MHz, DMSO-d₆) δ 7.98 (br s, 1H), 7.94 (d, J=8.1 Hz, 1H), 7.74 (d, J=7.9 Hz, 2H), 7.69 (dd, J=8.1, 1.5 Hz, 1H), 7.64 (s, 1H), 6.95 (d, J=9.0 Hz, 2H), 6.69 (s, 2H), 4.07 (dt, J=6.2, 2.9 Hz, 1H), 3.42 (td, J=6.5, 2.7 Hz, 2H), 3.37 (br d, J=3.1 Hz, 1H), 3.03-2.95 (m, 3H), 2.81 (br d, J=10.6 Hz, 1H), 2.66 (br d, J=10.8 Hz, 1H), 2.22 (dd, J=11.0, 3.5 Hz, 1H), 2.20 (s, 3H), 2.03 (td, J=11.2, 3.4 Hz, 1H), 1.05 (d, J=6.5 Hz, 3H); LCMS: [M+H]⁺=447.08.

### Example 53: (R)-6-(3-amino-6-(4-(2,4-dimethylpiperazin-1-yl)phenyl)-5-fluoropyrazin-2-yl)-7-fluoro-3,4-dihydroisoquinolin-1(2H)-one (I-53)

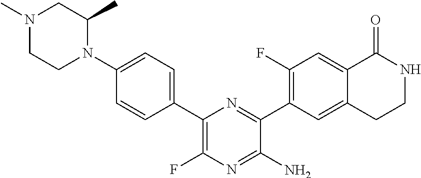 [SMILES: C[C@@H]1CN(C)CCN1c1ccc(-c2nc(-c3cc4c(cc3F)C(=O)NCC4)c(N)nc2F)cc1]

Prepared from 6-(3-amino-6-bromo-5-fluoropyrazin-2-yl)-7-fluoro-3,4-dihydroisoquinolin-1(2H)-one (107 mg, 0.300 mmol) and (R)-2,4-dimethyl-1-(4-(4,4,5,5-tetramethyl-1,3,2-dioxaborolan-2-yl)phenyl)piperazine (114 mg, 0.36 mmol) to give the title compound as a yellow solid (51 mg, 34.8% yield). ¹H NMR (500 MHz, DMSO-d₆) δ 8.15 (br s, 1H), 7.69 (d, J=7.9 Hz, 2H), 7.64 (d, J=10.3 Hz, 1H), 7.51 (d, J=6.8 Hz, 1H), 6.94 (d, J=9.0 Hz, 2H), 6.65 (s, 2H), 4.06 (dt, J=6.2, 2.9 Hz, 1H), 3.42 (td, J=6.5, 2.7 Hz, 2H), 3.38-3.35 (m, 1H), 3.02-2.91 (m, 3H), 2.81 (br d, J=10.6 Hz, 1H), 2.65 (br d, J=10.9 Hz, 1H), 2.22 (dd, J=11.1, 3.6 Hz, 1H), 2.19 (s, 3H), 2.03 (td, J=11.3, 3.5 Hz, 1H), 1.04 (d, J=6.5 Hz, 3H); LCMS: [M+1]+=465.06.

### Example 54: 6-(3-amino-5-fluoro-6-(4-(4-methylpiperazin-1-yl)phenyl)pyrazin-2-yl)-7-fluoro-3,4-dihydroisoquinolin-1(2H)-one (I-54)

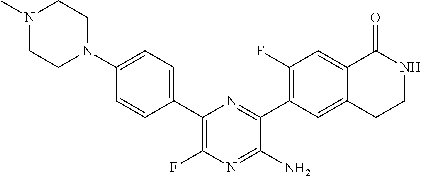 [SMILES: CN1CCN(c2ccc(-c3nc(-c4cc5c(cc4F)C(=O)NCC5)c(N)nc3F)cc2)CC1]

Prepared from 4-(4-methylpiperazin-1-yl)phenylboronic acid, pinacol ester (94 mg, 0.311 mmol) 4-(4-methylpiperazin-1-yl)phenylboronic acid, pinacol ester (94 mg, 0.311 mmol), to give the title compound as a yellow solid (87 mg, 73.1% yield). ¹H NMR (500 MHz, DMSO-d₆) δ 8.15 (br s, 1H), 7.69 (d, J=7.8 Hz, 2H), 7.64 (d, J=10.1 Hz, 1H), 7.51 (d, J=6.8 Hz, 1H), 7.00 (d, J=9.0 Hz, 2H), 6.68 (s, 2H), 3.42 (td, J=6.5, 2.7 Hz, 2H), 3.21-3.17 (m, 4H), 2.95 (br t, J=6.5 Hz, 2H), 2.48-2.44 (m, 4H), 2.23 (s, 3H); LCMS: [M+H]⁺=451.14.

### Example 55: 6-(3-amino-6-(4-(4-ethylpiperazin-1-yl)phenyl)-5-fluoropyrazin-2-yl)-3,4-dihydroisoquinolin-1(2H)-one (I-55)

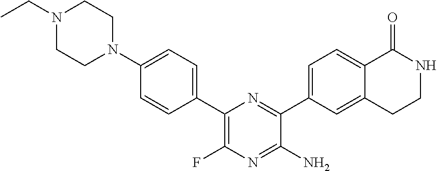 [SMILES: CCN1CCN(c2ccc(-c3nc(-c4ccc5c(c4)CCNC5=O)c(N)nc3F)cc2)CC1]

Prepared from 6-(3-amino-6-bromo-5-fluoropyrazin-2-yl)-3,4-dihydroisoquinolin-1(2H)-one (79 mg, 0.234 mmol) and (4-(4-ethylpiperizine-1-yl)phenyl)boronic acid pinacol ester (89 mg, 0.281 mmol) to give the title compound as a yellow solid, (84 mg, 75% yield). ¹H NMR (500 MHz, DMSO-d₆) δ 7.98 (br s, 1H), 7.95 (d, J=7.9 Hz, 1H), 7.74 (d, J=7.8 Hz, 2H), 7.69 (dd, J=8.0, 1.5 Hz, 1H), 7.64 (s, 1H), 7.01 (d, J=9.0 Hz, 2H), 6.71 (s, 2H), 3.42 (td, J=6.5, 2.7 Hz, 2H), 3.21-3.17 (m, 4H), 2.98 (t, J=6.5 Hz, 2H), 2.53-2.51 (m, 4H), 2.37 (q, J=7.1 Hz, 2H), 1.03 (t, J=7.2 Hz, 3H); LCMS: [M+H]⁺=447.08.

### Example 56: 6-(3-amino-6-(4-(4-ethylpiperazin-1-yl)phenyl)-5-fluoropyrazin-2-yl)-7-fluoro-3,4-dihydroisoquinolin-1(2H)-one (I-56)

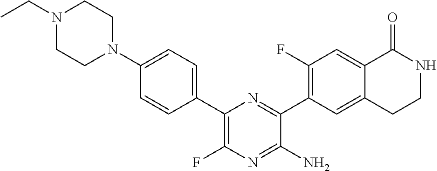 [SMILES: CCN1CCN(c2ccc(-c3nc(-c4cc5c(cc4F)C(=O)NCC5)c(N)nc3F)cc2)CC1]

Prepared from 6-(3-amino-6-bromo-5-fluoropyrazin-2-yl)-7-fluoro-3,4-dihydroisoquinolin-1(2H)-one (91 mg, 0.256 mmol) and (4-(4-ethylpiperizine-1-yl)phenyl)boronic acid pinacol ester (97 mg, 0.307 mmol) to give the title compound as a yellow solid, (94 mg, 74.2% yield). ¹H NMR (500 MHz, DMSO-d₆) δ 8.15 (br s, 1H), 7.70 (d, J=7.7 Hz, 2H), 7.64 (d, J=10.1 Hz, 1H), 7.51 (d, J=6.8 Hz, 1H), 7.00 (d, J=9.0 Hz, 2H), 6.68 (s, 2H), 3.42 (td, J=6.5, 2.8 Hz, 2H), 3.20 (br s, 4H), 2.95 (br t, J=6.4 Hz, 2H), 2.52 (br s, 4H), 2.39 (br d, J=6.8 Hz, 2H), 1.04 (t, J=7.2 Hz, 3H); LCMS: [M+H]⁺=465.14.

### Example 57: 6-(3-amino-5-fluoro-6-(4-((3R,5S)-3,4,5-trimethylpiperazin-1-yl)phenyl)pyrazin-2-yl)-3,4-dihydroisoquinolin-1(2H)-one (I-57)

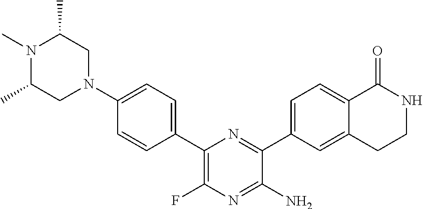 [SMILES: C[C@@H]1CN(c2ccc(-c3nc(-c4ccc5c(c4)CCNC5=O)c(N)nc3F)cc2)C[C@H](C)N1C]

Prepared from 6-(3-amino-6-bromo-5-fluoropyrazin-2-yl)-3,4-dihydroisoquinolin-1(2H)-one (101 mg, 0.300 mmol) and (2R,6S)-1,2,6-trimethyl-4-(4-(4,4,5,5-tetramethyl-1,3,2-dioxaborolan-2-yl)phenyl)piperazine (119 mg, 0.36 mmol, prepared through similar methods as described earlier) to give the title compound as a yellow solid (14 mg, 10.1% yield). ¹H NMR (500 MHz, DMSO-d₆) δ 7.98 (br s, 1H), 7.94 (d, J=7.9 Hz, 1H), 7.73 (d, J=7.8 Hz, 2H), 7.68 (dd, J=8.1, 1.5 Hz, 1H), 7.64 (s, 1H), 7.01 (d, J=9.0 Hz, 2H), 6.71 (s, 2H), 3.63 (br d, J=11.0 Hz, 2H), 3.42 (td, J=6.5, 2.6 Hz, 2H), 2.98 (t, J=6.5 Hz, 2H), 2.43 (t, J=11.4 Hz, 2H), 2.28-2.21 (m, 2H), 2.19 (s, 3H), 1.08 (d, J=6.1 Hz, 6H); 19F NMR (471 MHz, DMSO-d6) δ −81.96 (s, 1F).

### Example 58: 6-(3-amino-5-fluoro-6-(4-((3R,5S)-3,4,5-trimethylpiperazin-1-yl)phenyl)pyrazin-2-yl)-7-fluoro-3,4-dihydroisoquinolin-1(2H)-one (I-58)

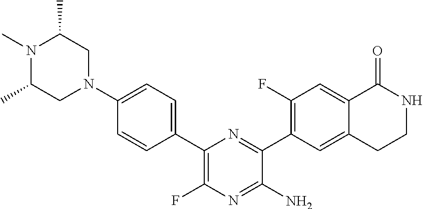 [SMILES: C[C@@H]1CN(c2ccc(-c3nc(-c4cc5c(cc4F)C(=O)NCC5)c(N)nc3F)cc2)C[C@H](C)N1C]

Prepared from 6-(3-amino-6-bromo-5-fluoropyrazin-2-yl)-7-fluoro-3,4-dihydroisoquinolin-1(2H)-one (107 mg, 0.300 mmol), (2R,6S)-1,2,6-trimethyl-4-(4-(4,4,5,5-tetramethyl-1,3,2-dioxaborolan-2-yl)phenyl)piperazine (119 mg, 0.36 mmol) to give the title compound as a yellow solid, (57 mg, 39.7% yield). ¹H NMR (500 MHz, DMSO-d₆) δ 8.15 (br s, 1H), 7.68 (d, J=7.7 Hz, 2H), 7.64 (d, J=10.1 Hz, 1H), 7.50 (d, J=6.8 Hz, 1H), 6.99 (d, J=9.0 Hz, 2H), 6.67 (s, 2H), 3.62 (br d, J=10.9 Hz, 2H), 3.42 (td, J=6.5, 2.7 Hz, 2H), 2.95 (br t, J=6.5 Hz, 2H), 2.42 (t, J=11.4 Hz, 2H), 2.27-2.20 (m, 2H), 2.18 (s, 3H), 1.07 (d, J=6.1 Hz, 6H); LCMS: [M+1 ]⁺=479.51.

### Example 59: 6-(3-amino-6-(3-((dimethylamino)methyl)-4-(tetrahydro-2H-pyran-4-yl)phenyl)-5-fluoropyrazin-2-yl)-3,4-dihydroisoquinolin-1(2H)-one (I-59)

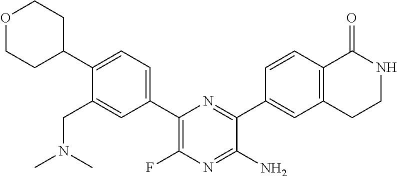 [SMILES: CN(C)Cc1cc(-c2nc(-c3ccc4c(c3)CCNC4=O)c(N)nc2F)ccc1C1CCOCC1]

### Step 1: 1-(5-bromo-2-(tetrahydro-2H-pyran-4-yl)phenyl)-N,N-dimethylmethanamine

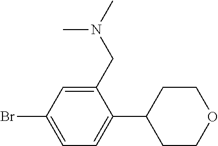 [SMILES: CN(C)Cc1cc(Br)ccc1C1CCOCC1]

Dimethylamine, 2.0M solution in THF (2.55 mL, 5.10 mmol) was added to a solution of 5-bromo-2-(tetrahydro-2H-pyran-4-yl)benzaldehyde (490 mg, 1.274 mmol) and acetic acid, (7.29 μl, 0.127 mmol) in CH₂Cl₂ (10 mL) at RT. After 5 min of stirring sodium triacetoxyborohydride (1080 mg, 5.10 mmol) was added and the suspension was stirred for 30 minutes at RT. The reaction mixture was basified with 1N NaOH aqueous solution, the organic phase was separated, aqueous phase was extracted with CH₂Cl₂ (5 mL), the combined org phase was dried over Na₂SO₄ and concentrated to yield the crude product as a white solid (419 mg, 91% yield). LCMS: [M+H]⁺=298.34

### Step 2: N,N-dimethyl-1-(2-(tetrahydro-2H-pyran-4-yl)-5-(4,4,5,5-tetramethyl-1,3,2-dioxaborolan-2-yl)phenyl)methanamine

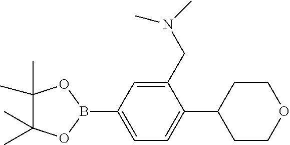 [SMILES: CN(C)Cc1cc(B2OC(C)(C)C(C)(C)O2)ccc1C1CCOCC1]

Prepared from 1-(5-bromo-2-(tetrahydro-2H-pyran-4-yl)phenyl)-N,N-dimethylmethanamine (120 mg, 0.350 mmol). The title compound was isolated as a brown solid (121 mg, 90% yield based on 90% purity); LCMS: [M+H]⁺=346.36

### Step 3: 6-(3-amino-6-(3-((dimethylamino)methyl)-4-(tetrahydro-2H-pyran-4-yl)phenyl)-5-fluoropyrazin-2-yl)-3,4-dihydroisoquinolin-1(2H)-one

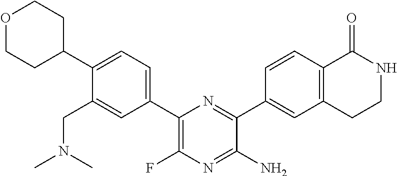 [SMILES: CN(C)Cc1cc(-c2nc(-c3ccc4c(c3)CCNC4=O)c(N)nc2F)ccc1C1CCOCC1]

Prepared from 6-(3-amino-6-bromo-5-fluoropyrazin-2-yl)-3,4-dihydroisoquinolin-1(2H)-one (45 mg, 0.133 mmol) and N,N-dimethyl-1-(2-(tetrahydro-2H-pyran-4-yl)-5-(4,4,5,5-tetramethyl-1,3,2-dioxaborolan-2-yl)phenyl)methanamine (57.6 mg, 0.167 mmol) to give the title compound as a beige solid (37 mg, 55% yield). ¹H NMR (500 MHz, METHANOL-d₄) δ ppm 8.09 (d, J=8.07 Hz, 1H), 7.84-7.89 (m, 2H), 7.79 (dd, J=8.07, 1.47 Hz, 1H), 7.73 (s, 1H), 7.43 (d, J=8.19 Hz, 1H), 4.08 (dd, J=11.25, 3.79 Hz, 2H), 3.57-3.66 (m, 6H), 3.26-3.30 (m, 1H), 3.11 (t, J=6.66 Hz, 2H), 2.30 (s, 6H), 1.84-1.93 (m, 2H), 1.74 (d, J=11.62 Hz, 2H); LCMS: [M+H]⁺=476.42

### Example 60: 6-(3-amino-6-(3-((dimethylamino)methyl)-4-(tetrahydro-2H-pyran-4-yl)phenyl)-5-fluoropyrazin-2-yl)-7-fluoro-3,4-dihydroisoquinolin-1(2H)-one (I-60)

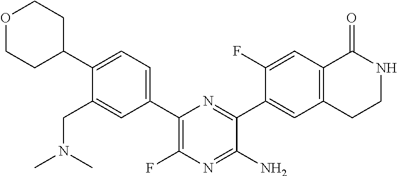 [SMILES: CN(C)Cc1cc(-c2nc(-c3cc4c(cc3F)C(=O)NCC4)c(N)nc2F)ccc1C1CCOCC1]

Prepared from 6-(3-amino-6-bromo-5-fluoropyrazin-2-yl)-7-fluoro-3,4-dihydroisoquinolin-1(2H)-one (45 mg, 0.127 mmol) and N,N-dimethyl-1-(2-(tetrahydro-2H-pyran-4-yl)-5-(4,4,5,5-tetramethyl-1,3,2-dioxaborolan-2-yl)phenyl)methanamine (54.7 mg, 0.158 mmol) to give the title compound as a beige solid (36 mg, 55% yield). ¹H NMR (500 MHz, METHANOL-d₄) δ ppm 7.73-7.87 (m, 3H), 7.73-7.87 (m, 3H), 7.54 (d, J=6.72 Hz, 1H), 7.41 (d, J=8.19 Hz, 1H), 4.07 (dd, J=11.19, 3.61 Hz, 2H), 3.55-3.65 (m, 6H), 3.26-3.32 (m, 1H), 3.06 (t, J=6.60 Hz, 2H), 2.26-2.32 (m, 6H), 1.82-1.95 (m, 2H), 1.71-1.78 (m, 2H); LCMS: [M+H]⁺=494.34

### Example 61: 6-(3-amino-5-fluoro-6-(4-(2-methyl-2,7-diazaspiro[3.5]nonan-7-yl)phenyl)pyrazin-2-yl)-7-fluoro-3,4-dihydroisoquinolin-1(2H)-one (I-61)

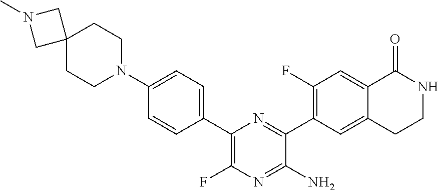 [SMILES: CN1CC2(CCN(c3ccc(-c4nc(-c5cc6c(cc5F)C(=O)NCC6)c(N)nc4F)cc3)CC2)C1]

Prepared from 6-(3-amino-6-chloro-5-fluoropyrazin-2-yl)-7-fluoro-3,4-dihydroisoquinolin-1(2H)-one (36.9 mg, 0.119 mmol) and 2-methyl-7-(4-(4,4,5,5-tetramethyl-1,3,2-dioxaborolan-2-yl)phenyl)-2,7-diazaspiro[3.5]nonane (33.9 mg, 0.099 mmol) to give the title compound (6.4 mg, 12.7% yield). ¹H NMR (500 MHz, DMSO-d₆) δ=8.14 (br s, 1H), 7.68 (d, J=7.8 Hz, 2H), 7.64 (d, J=10.3 Hz, 1H), 7.50 (d, J=6.8 Hz, 1H), 6.99 (d, J=9.0 Hz, 2H), 6.65 (s, 2H), 3.42 (dt, J=2.8, 6.5 Hz, 2H), 3.24-3.09 (m, 5H), 2.99-2.91 (m, 6H), 2.23 (s, 3H), 1.80-1.71 (m, 4H); LCMS: [M+H]⁺=491.46.

### Example 62: 6-(3-amino-5-fluoro-6-(4-(4-isopropylpiperazin-1-yl)phenyl)pyrazin-2-yl)-4-methylisoquinolin-1(2H)-one (I-62)

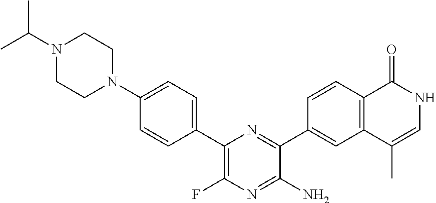 [SMILES: Cc1c[nH]c(=O)c2ccc(-c3nc(-c4ccc(N5CCN(C(C)C)CC5)cc4)c(F)nc3N)cc12]

### Step 1 (E)-3-(3-bromophenyl)but-2-enoic acid

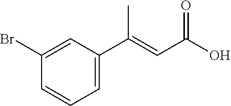

To THF (150 mL) at 0° C. under nitrogen atmosphere, sodium hydride (60%, 6 g, 150.71 mmol) was add portion wise. To this, triethyl phosphonoacetate (21.47 mL, 113.06 mmol) was added dropwise and stirred at 0° C. for 10 min. The resulting reaction mass was added dropwise to a solution of 1-(3-bromophenyl)ethan-1-one (15 g, 75.35 mmol) in THF (150 mL) and the reaction mass was reflux for 16 h. After completion of the reaction, reaction mass was diluted with water (500 mL) and extracted with DCM (3×500 mL). The organic layer was dried over anhydrous Na₂SO₄ and concentrated under vacuum to afford a yellow oil. The oil was dissolved in methanol (150 mL) and sodium hydroxide (9.04 g, 226.13 mmol) and water (75 mL) were added and the reaction mass was heated at 50° C. for 2 h. After completion of the reaction, the reaction mass was concentrated to evaporate the organics. The aqueous solution was acidified by aqueous 2M HCl and extracted with ethyl acetate (2×500 mL). The combined organic layer was dried over anhydrous sodium sulfate and concentrated under vacuum to afford the product (7 g, 39%) as yellow solid LCMS: [M−2H]⁻=238.9.

### Step 2: (E)-3-(3-bromophenyl)but-2-enoyl chloride

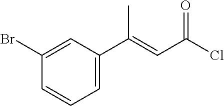

To a cooled solution of (E)-3-(3-bromophenyl)but-2-enoic acid (6 g, 24.88 mmol) in DCM (60 mL) and DMF (0.2 mL) at 0° C., oxaloyl chloride (2.59 mL, 29.86 mmol) was added dropwise and the reaction mass was allowed to warm to RT and stirred at same temperature for 3 h. After completion the reaction, reaction mass was concentrated and azeotrope with toluene (2×20 mL) and DCM (2×20 mL) to get the crude product which was used as is in the next step.

### Step 3: (E)-3-(3-bromophenyl)but-2-enoyl azide

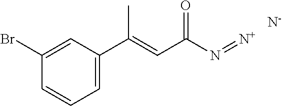

To a cooled solution of crude of (E)-3-(3-bromophenyl)but-2-enoyl chloride (6 g, 23.11 mmol) in 1,4 dioxane (60 mL) at 0° C., a suspension of sodium azide (2.26 g, 34.47 mmol) in 1:1 mixture of 1,4-dioxane and water (20 mL) was added and the reaction mas was gradually warm to room temperature and stirred at same temperature for a 3 h. After completion reaction, reaction mass was diluted with water (70 mL) and extracted with diethyl ether (2×200 mL). The combined organic mixture was back washed with saturated sodium bicarbonate solution (3×200 mL) and water (3×200 mL) and dried over anhydrous Na₂SO₄ and organic layer was directly used for next step.

### Step 4: 6-bromo-4-methylisoquinolin-1(2H)-one

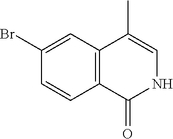

To the ether layer of (E)-3-(3-bromophenyl)but-2-enoyl azide is treated with 1,2 dichlorobenzene (15 mL) and the ether was removed under vacuum to give a solution of (E)-3-(3-bromophenyl)but-2-enoyl azide in 1,2 dichlorobenzene. The acyl azide solution in 1,2 dichlorobenzene was added dropwise over 30 min to a solution of iodine (3 crystal) in 1,2 dichlorobenzene (15 mL) at 120° C. After completion of addition, reaction mass was stirred at 190° C. for 16 h. After completion of the reaction, reaction mass was allowed to cool room temperature and added to hexane (800 mL). The suspension stirred for 1 h and the solid thus obtained was filtration, washed with ethyl acetate (20 mL) and DCM (20 mL) and dried under vacuum to give the product (1.25 g, 23%) as pale yellow solid. LCMS: [M+2]⁺=240.8.

### Step 5: 4-methyl-6-(4,4,5,5-tetramethyl-1,3,2-dioxaborolan-2-yl)isoquinolin-1(2H)-one

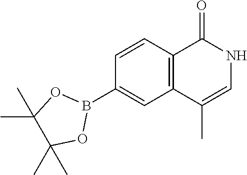 [SMILES: Cc1c[nH]c(=O)c2ccc(B3OC(C)(C)C(C)(C)O3)cc12]

To 6-bromo-4-methylisoquinolin-1(2H)-one (742 mg, 3.12 mmol), bis(pinacolato)diboron (871 mg, 3.43 mmol), and potassium acetate (918 mg, 9.35 mmol), was added anhydrous 1,4-dioxane (20 ml). The system was flushed with nitrogen then [1,12-bis(diphenylphosphino)ferrocene]dichloropalladium(II) (228 mg, 0.312 mmol) was added. The mixture was flushed with nitrogen then heated at 100° C. for 2 hours. The reaction was diluted with acetonitrile, filtered through a pad of celite, concentrated in vacuo and used crude in the next step (120 mg, 0.341 mmol, 100%) as a mixture of boronate and boronic acid. LCMS: [M+H]⁺=285.99 Boronate, 204.29 Boronic acid.

### Step 6: 6-fluoro-5-(4-(4-isopropylpiperazin-1-yl)phenyl)pyrazin-2-amine

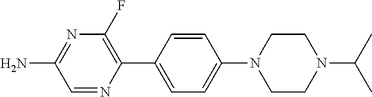 [SMILES: CC(C)N1CCN(c2ccc(-c3ncc(N)nc3F)cc2)CC1]

To a degassed solution of 1-isopropyl-4-(4-(4,4,5,5-tetramethyl-1,3,2-dioxaborolan-2-yl) phenyl) piperazine (5 g, 15.15 mmol) in dioxane (25 mL) and water (25 mL), K₂CO₃ (5.2 g, 37.75 mmol) and 5-bromo-6-fluoropyrazin-2-amine (3.96 g, 16.66 mmol) were added and the reaction mass was further degassed for 10 min using Nitrogen. Tetrakis (0.875 g, 0.75 mmol) was added under nitrogen atmosphere and the mixture was stirred at 120° C. for 4 h, then diluted with water (100 mL) and extracted with EtOAc (3×100 mL). The combined organic layer was dried over Na₂SO₄, concentrated under vacuum and the resulting crude product was purified by column chromatography eluting with 4% MeOH in DCM to afford the product (2.5 g, 31.3%) as a dark brown solid. LCMS: [M+H]⁺=316.24.

### Step 7: 3-bromo-6-fluoro-5-(4-(4-isopropylpiperazin-1-yl)phenyl)pyrazin-2-amine

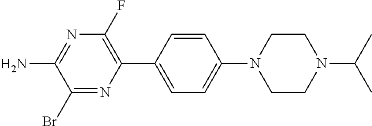 [SMILES: CC(C)N1CCN(c2ccc(-c3nc(Br)c(N)nc3F)cc2)CC1]

A mixture of 6-fluoro-5-(4-(4-isopropylpiperazin-1-yl)phenyl)pyridine-2-amine (6.7 g, 21.33 mmol) and N-bromosuccinimide (3.77 g, 21.33 mmol) in DMF (60 mL) was stirred at RT for 5 h. After 5 h, additional N-bromosuccinimide (1.88 g, 10.66 mmol) was added and the mixture was stirred at RT for 16 h. After 16 h, another portion of N-bromosuccinimide (1.13 g, 6.4 mmol) was added and the mixture was stirred at RT for an additional 4 h. The reaction was diluted with saturated NaHCO₃ solution (200 mL) and extracted with EtOAc (3×150 mL). The combined organic was dried over anhydrous Na₂SO₄, concentrated under vacuum and purified by column chromatography. The product was eluted with 15-20% EtOAc in hexanes to afford the product (1.1 g, 13%) as a brownish yellow solid. LCMS: [M+H]⁺=394.2.

### Step 8: 6-(3-amino-5-fluoro-6-(4-(4-isopropylpiperazin-1-yl)phenyl)pyrazin-2-yl)-4-methylisoquinolin-1(2H)-one

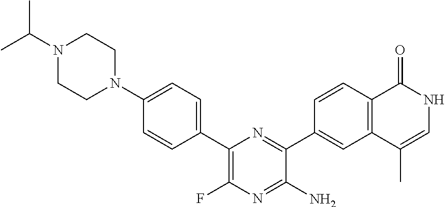 [SMILES: Cc1c[nH]c(=O)c2ccc(-c3nc(-c4ccc(N5CCN(C(C)C)CC5)cc4)c(F)nc3N)cc12]

Prepared from (4-methyl-1-oxo-1,2-dihydroisoquinolin-6-yl)boronic acid (48.2 mg, 0.237 mmol) and 3-bromo-6-fluoro-5-(4-(4-isopropylpiperazin-1-yl)phenyl)pyrazin-2-amine (72 mg, 0.183 mmol) to afford the title compound (19.8 mg, 23% yield) as a yellow solid. ¹H NMR (500 MHz, DMSO-d₆) δ ppm 11.12 (d, J=5.50 Hz, 1H), 8.33 (d, J=8.31 Hz, 1H), 7.99 (s, 1H), 7.84 (d, J=8.19 Hz, 1H), 7.76 (d, J=8.07 Hz, 2H), 7.06 (d, J=4.77 Hz, 1H), 7.02 (d, J=8.93 Hz, 2H), 6.80 (s, 2H), 3.19 (br. s., 4H), 2.68 (br. s., 1H), 2.59 (br. s., 4H), 2.26 (s, 3H), 1.02 (d, J=6.36 Hz, 6H); LCMS [M+H]⁺=473.38.

### Example 63: 7-(3-amino-5-fluoro-6-(4-(4-isopropylpiperazin-1-yl)phenyl)pyrazin-2-yl)-6-fluoro-2-methylquinazolin-4(3H)-one (I-63)

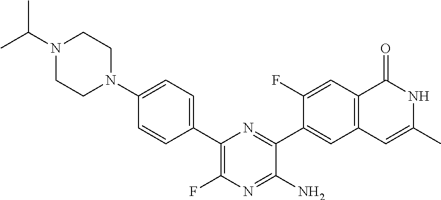 [SMILES: Cc1cc2cc(-c3nc(-c4ccc(N5CCN(C(C)C)CC5)cc4)c(F)nc3N)c(F)cc2c(=O)[nH]1]

### Step 1: 7-bromo-6-fluoro-2-methylquinazolin-4(3H)-one

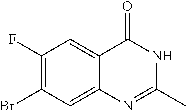

2-Amino-4-bromo-5-fluorobenzoic acid (1.0 g, 4.27 mmol) was taken up in acetic anhydride (5.65 mL, 59.8 mmol) and heated at 130° C. overnight. The reaction mixture was cooled to RT and stirred for two hours. The precipitated solid was filtered, washed with cold ether, and dried under vacuum to give 2-acetamido-4-bromo-5-fluorobenzoic acid which was used in the next step without further purification. LCMS [M−H]⁻=273.89. The 2-acetamido-4-bromo-5-fluorobenzoic acid was take up in 27% aqueous ammonium hydroxide solution (3.33 ml, 23.07 mmol) and heated in a sealed tube at 80° C. for 4 hours. After cooling to RT, the solid was filtered, washed with water, and dried under vacuum to afford the product (132 mg, 0.513 mmol, 12.02% yield) as a white solid. LCMS [M+H]⁺=257.09.

### Step 2: (6-fluoro-2-methyl-4-oxo-3,4-dihydroquinazolin-7-yl)boronic acid

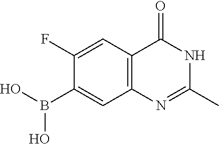 [SMILES: Cc1nc2cc(B(O)O)c(F)cc2c(=O)[nH]1]

Prepared from 7-bromo-6-fluoro-2-methylquinazolin-4(3H)-one (86 mg, 0.335 mmol) to give a mixture of boronate and boronic acid which was used in the next step without further purification. LCMS[M+H]⁺=289.31 Boronate, 207.18 Boronic acid.

### Step 3: 7-(3-amino-5-fluoro-6-(4-(4-isopropylpiperazin-1-yl)phenyl)pyrazin-2-yl)-6-fluoro-2-methylquinazolin-4(3H)-one

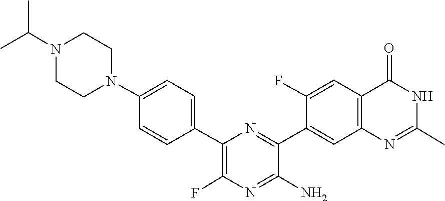 [SMILES: Cc1nc2cc(-c3nc(-c4ccc(N5CCN(C(C)C)CC5)cc4)c(F)nc3N)c(F)cc2c(=O)[nH]1]

Prepared from (6-fluoro-2-methyl-4-oxo-3,4-dihydroquinazolin-7-yl)boronic acid (35.8 mg, 0.161 mmol) and 3-bromo-6-fluoro-5-(4-(4-isopropylpiperazin-1-yl)phenyl)pyrazin-2-amine (53 mg, 0.134 mmol) to give the product (46.5 mg, 70.4% yield) as a yellow solid. ¹H NMR (500 MHz, DMSO-d₆) δ ppm 12.36 (br. s., 1H), 7.85 (d, J=9.54 Hz, 1H), 7.74 (d, J=6.36 Hz, 1H), 7.70 (d, J=7.95 Hz, 2H), 6.99 (d, J=8.93 Hz, 2H), 6.72 (s, 2H), 3.15-3.20 (m, 4H), 2.64-2.72 (m, 1H), 2.55-2.61 (m, 4H), 2.38 (s, 3H), 1.00 (d, J=6.48 Hz, 6H); LCMS [M+H]⁺=492.34.

### Example 64: 6-(3-amino-6-(3-((dimethylamino)methyl)-4-morpholinophenyl)-5-fluoropyrazin-2-yl)-3,4-dihydroisoquinolin-1(2H)-one (I-64)

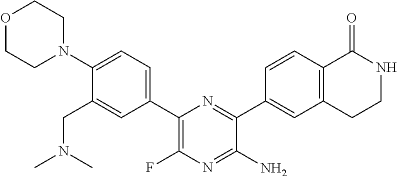 [SMILES: CN(C)Cc1cc(-c2nc(-c3ccc4c(c3)CCNC4=O)c(N)nc2F)ccc1N1CCOCC1]

### Step 1: 1-(5-bromo-2-morpholinophenyl)-N,N-dimethylmethanamine

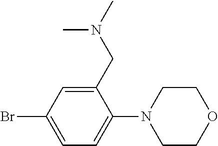 [SMILES: CN(C)Cc1cc(Br)ccc1N1CCOCC1]

Prepared from 5-bromo-2-morpholinobenzaldehyde (500 mg, 1.851 mmol) and dimethylamine, 2.0 M solution in THF (3.70 ml, 7.40 mmol) to give the title compound as a pale-yellow solid (543 mg, 98%). LCMS: [M+H]⁺=299.15

### Step 2: N,N-dimethyl-1-(2-morpholino-5-(4,4,5,5-tetramethyl-1,3,2-dioxaborolan-2-yl)phenyl)methanamine

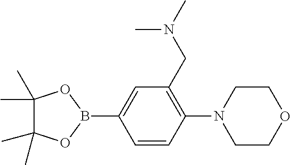 [SMILES: CN(C)Cc1cc(B2OC(C)(C)C(C)(C)O2)ccc1N1CCOCC1]

Prepared from 1-(5-bromo-2-morpholinophenyl)-N,N-dimethylmethanamine (125 mg, 0.418 mmol) to give the title compound as a brown solid which was taken to the next step without any purification. (145 mg, 90% yield based on 90% purity); LCMS: [M+H]⁺=347.30.

### Step 3: 6-(3-amino-6-(3-((dimethylamino)methyl)-4-morpholinophenyl)-5-fluoropyrazin-2-yl)-3,4-dihydroisoquinolin-1(2H)-one

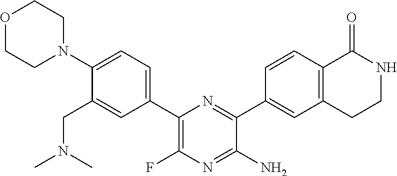 [SMILES: CN(C)Cc1cc(-c2nc(-c3ccc4c(c3)CCNC4=O)c(N)nc2F)ccc1N1CCOCC1]

Prepared from 6-(3-amino-6-bromo-5-fluoropyrazin-2-yl)-3,4-dihydroisoquinolin-1(2H)-one (50 mg, 0.148 mmol) and N,N-dimethyl-1-(2-morpholino-5-(4,4,5,5-tetramethyl-1,3,2-dioxaborolan-2-yl)phenyl)methanamine (68.1 mg, 0.185 mmol) to give the title compound as a beige solid (26 mg, 35% yield). ¹H NMR (500 MHz, DMSO-d₆) b ppm 7.94-8.02 (m, 2H), 7.88 (br. s., 1H), 7.74 (d, J=8.19 Hz, 1H), 7.68 (d, J=8.07 Hz, 1H), 7.64 (s, 1H), 7.18 (d, J=8.44 Hz, 1H), 6.82 (br. s., 2H), 3.76 (br. s., 4H), 3.47 (br. s., 2H), 3.42-3.45 (m, 2H), 2.95-3.01 (m, 6H), 2.19-2.21 (m, 6H), 2.20 (s, 6H); LCMS: [M+H]⁺=477.49

### Example 65: 6-(3-amino-6-(3-((dimethylamino)methyl)-4-morpholinophenyl)-5-fluoropyrazin-2-yl)-7-fluoro-3,4-dihydroisoquinolin-1(2H)-one (I-65)

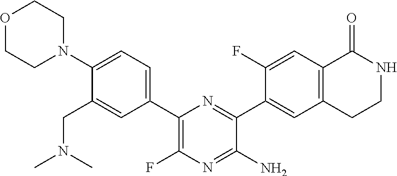 [SMILES: CN(C)Cc1cc(-c2nc(-c3cc4c(cc3F)C(=O)NCC4)c(N)nc2F)ccc1N1CCOCC1]

Prepared from 6-(3-amino-6-bromo-5-fluoropyrazin-2-yl)-7-fluoro-3,4-dihydroisoquinolin-1(2H)-one (50 mg, 0.141 mmol and N,N-dimethyl-1-(2-morpholino-5-(4,4,5,5-tetramethyl-1,3,2-dioxaborolan-2-yl)phenyl)methanamine (64.6 mg, 0.176 mmol) to give the title compound was isolated as a beige solid (26 mg, 35.5% yield). ¹H NMR (500 MHz, DMSO-d₆) δ ppm 8.21 (br. s., 1H), 7.90 (s, 1H), 7.75 (d, J=8.44 Hz, 1H), 7.71 (d, J=10.15 Hz, 1H), 7.57 (d, J=6.72 Hz, 1H), 7.22 (d, J=8.44 Hz, 1H), 6.84 (br. s., 2H), 3.74-3.87 (m, 4H), 3.52 (br. s., 2H), 3.47-3.51 (m, 2H), 2.94-3.09 (m, 6H), 2.12-2.35 (m, 6H); LCMS: [M+H]⁺=495.35.

### Example 66: 6-(3-amino-5-fluoro-6-(4-(4-isopropylpiperazin-1-yl)phenyl)pyrazin-2-yl)-4-fluoroisoquinolin-1(2H)-one (I-66)

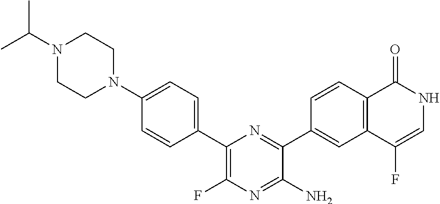 [SMILES: CC(C)N1CCN(c2ccc(-c3nc(-c4ccc5c(=O)[nH]cc(F)c5c4)c(N)nc3F)cc2)CC1]

From (4-fluoro-1-oxo-1,2-dihydroisoquinolin-6-yl)boronic acid (29.0 mg, 0.140 mmol) and 3-bromo-6-fluoro-5-(4-(4-isopropylpiperazin-1-yl)phenyl)pyrazin-2-amine (46 mg, 0.117 mmol) to give the product (15.4 mg, 28% yield) as a yellow solid. ¹H NMR (500 MHz, DMSO-d₆) δ ppm 11.19 (d, J=4.40 Hz, 1H), 8.32 (dd, J=8.31, 1.59 Hz, 1H), 8.06 (s, 1H), 7.95 (dd, J=8.38, 1.16 Hz, 1H), 7.75 (d, J=8.07 Hz, 2H), 7.40-7.46 (m, 1H), 7.02 (d, J=8.93 Hz, 2H), 6.84 (s, 2H), 3.18 (d, J=4.65 Hz, 4H), 2.68 (dt, J=12.72, 6.36 Hz, 1H), 2.58 (br. s., 4H), 1.01 (d, J=6.48 Hz, 6H); LCMS [M+H]⁺=477.36.

### Example 67: 6-(3-amino-5-fluoro-6-(4-(4-isopropylpiperazin-1-yl)phenyl)pyrazin-2-yl)-8-fluoro-3-methylisoquinolin-1(2H)-one (I-67)

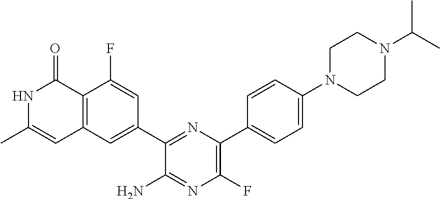 [SMILES: Cc1cc2cc(-c3nc(-c4ccc(N5CCN(C(C)C)CC5)cc4)c(F)nc3N)cc(F)c2c(=O)[nH]1]

### Step 1: 6-bromo-8-fluoro-3-methylisoquinolin-1(2H)-one

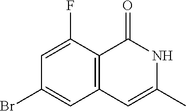

To a RBF was added copper(I) bromide (0.209 g, 1.455 mmol), Cs₂CO₃ (9.48 g, 29.1 mmol), 2,4-dibromo-6-fluorobenzamide (4.32 g, 14.55 mmol), propan-2-one (5.34 ml, 72.7 mmol) and dimethylsulfoxide (DMSO) (150 ml). The reaction was stirred and heated at 80° C. overnight. The reaction mixture was partitioned between brine (200 mL) and DCM (200 mL). The organic layer was separated, and the aqueous layer washed with DCM (2×100 mL). The combined organic layers were washed with brine, dried over anhydrous Na₂SO₄, and concentrated onto celite. The mixture was purified by flash chromatography (Biotage, silica gel) eluting with 0-100% EtOAc/Hexanes. The desired fractions were collected, concentrated and dried under vacuum to afford the product (2.41 g, 64.7% yield) as a pale-yellow solid. LCMS: [M+H]⁺=256.08.

### Step 2: (8-fluoro-3-methyl-1-oxo-1,2-dihydroisoquinolin-6-yl)boronic acid

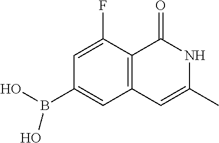 [SMILES: Cc1cc2cc(B(O)O)cc(F)c2c(=O)[nH]1]

Prepared from 6-bromo-8-fluoro-3-methylisoquinolin-1(2H)-one (73 mg, 0.285 mmol) to give a mixture of the boronic acid and boronate which was used in the next step without further purification. LCMS: [M+H]+ 222.23 Boronic acid, 304.23 Boronate

### Step 3: 6-(3-amino-5-fluoro-6-(4-(4-isopropylpiperazin-1-yl)phenyl)pyrazin-2-yl)-8-fluoro-3-methylisoquinolin-1(2H)-one

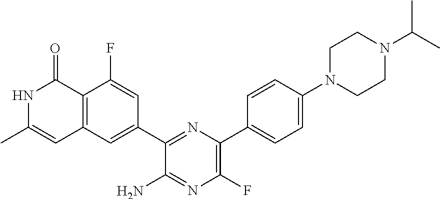 [SMILES: Cc1cc2cc(-c3nc(-c4ccc(N5CCN(C(C)C)CC5)cc4)c(F)nc3N)cc(F)c2c(=O)[nH]1]

Prepared from (8-fluoro-3-methyl-1-oxo-1,2-dihydroisoquinolin-6-yl)boronic acid (30.9 mg, 0.140 mmol) and 3-bromo-6-fluoro-5-(4-(4-isopropylpiperazin-1-yl)phenyl)pyrazin-2-amine (46 mg, 0.117 mmol) to give the title compound (11 mg, 19% yield) as a yellow solid. ¹H NMR (500 MHz, DMSO-d₆) δ ppm 11.29 (s, 1H), 7.75 (d, J=8.19 Hz, 2H), 7.67 (s, 1H), 7.39 (d, J=12.47 Hz, 1H), 7.01 (d, J=8.93 Hz, 2H), 6.83 (s, 2H), 6.41 (s, 1H), 3.19 (br. s., 4H), 2.65-2.72 (m, 1H), 2.59 (br. s., 4H), 2.21 (s, 3H), 1.01 (d, J=6.48 Hz, 6H); LCMS: [M+H]⁺=491.21.

### Example 68: 6-(3-amino-6-(4-(4-(4,4-difluorobutyl)piperazin-1-yl)phenyl)-5-fluoropyrazin-2-yl)-3,4-dihydroisoquinolin-1(2H)-one (I-68)

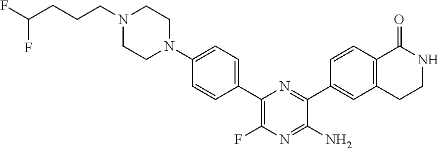 [SMILES: Nc1nc(F)c(-c2ccc(N3CCN(CCCC(F)F)CC3)cc2)nc1-c1ccc2c(c1)CCNC2=O]

### Step 1: 6-(3-amino-5-fluoro-6-(4-(piperazin-1-yl)phenyl)pyrazin-2-yl)-3,4-dihydroisoquinolin-1(2H)-one

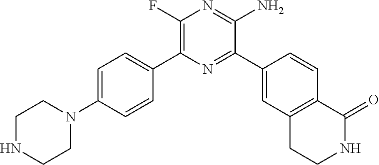 [SMILES: Nc1nc(F)c(-c2ccc(N3CCNCC3)cc2)nc1-c1ccc2c(c1)CCNC2=O]

Prepared from 6-(3-amino-6-bromo-5-fluoropyrazin-2-yl)-3,4-dihydroisoquinolin-1(2H)-one (70 mg, 0.208 mmol) and 4-piperazinylphenylboronic acid, pinacol ester (78 mg, 0.270 mmol) to give the product as a beige solid (82 mg, 94% yield). LCMS: [M+H]⁺=419.45

### Step 2: 6-(3-amino-6-(4-(4-(4,4-difluorobutyl)piperazin-1-yl)phenyl)-5-fluoropyrazin-2-yl)-3,4-dihydroisoquinolin-1(2H)-one

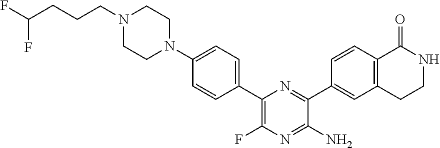 [SMILES: Nc1nc(F)c(-c2ccc(N3CCN(CCCC(F)F)CC3)cc2)nc1-c1ccc2c(c1)CCNC2=O]

A solution of N,N-diisopropylethylamine (0.278 ml, 1.596 mmol), 6-(3-amino-5-fluoro-6-(4-(piperazin-1-yl)phenyl)pyrazin-2-yl)-3,4-dihydroisoquinolin-1(2H)-one (85 mg, 0.203 mmol) and 4-bromo-1,1-difluorobutane (55.2 mg, 0.319 mmol) in N,N-Dimethylformamide (DMF) (1.5 ml) was heated at 50° C. for 6 h. The reaction mixture was stirred continuously with water (8 mL) for 5 min, the solid was filtered, the filter cake was washed a few times with water and then dried under high vac to yield the title compound was isolated as a pale-yellow solid (79 mg, 95% purity). ¹H NMR (500 MHz, METHANOL-d₄) b ppm 7.96 (d, J=7.95 Hz, 1H), 7.75 (d, J=8.19 Hz, 2H), 7.67 (d, J=7.95 Hz, 1H), 7.61 (s, 1H), 6.94 (d, J=8.93 Hz, 2H), 5.72-5.97 (m, 1H), 3.45-3.49 (m, 2H), 3.18 (d, J=5.26 Hz, 4H), 2.98 (t, J=6.60 Hz, 2H), 2.55-2.61 (m, 4H), 2.37-2.42 (m, 2H), 1.75-1.84 (m, 2H), 1.59-1.66 (m, 2H); LCMS: [M+H]⁺=511.39.

### Example 69: 6-(3-amino-6-(4-(4-(4,4-difluorobutyl)piperazin-1-yl)phenyl)-5-fluoropyrazin-2-yl)-7-fluoro-3,4-dihydroisoquinolin-1(2H)-one (I-69)

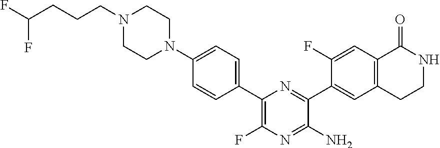 [SMILES: Nc1nc(F)c(-c2ccc(N3CCN(CCCC(F)F)CC3)cc2)nc1-c1cc2c(cc1F)C(=O)NCC2]

### Step 1: tert-butyl 4-(4-(5-amino-3-fluoro-6-(7-fluoro-1-oxo-1,2,3,4-tetrahydroisoquinolin-6-yl)pyrazin-2-yl)phenyl)piperazine-1-carboxylate

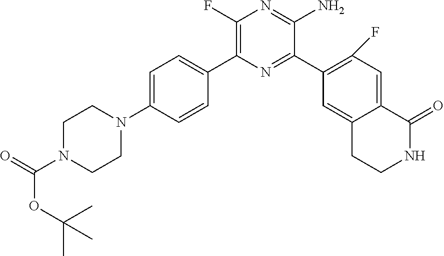 [SMILES: CC(C)(C)OC(=O)N1CCN(c2ccc(-c3nc(-c4cc5c(cc4F)C(=O)NCC5)c(N)nc3F)cc2)CC1]

Prepared from 6-(3-amino-6-bromo-5-fluoropyrazin-2-yl)-7-fluoro-3,4-dihydroisoquinolin-1(2H)-one (250 mg, 0.704 mmol) and tert-butyl 4-[4-(4,4,5,5-tetramethyl-1,3,2-dioxaborolan-2-yl)phenyl]tetrahydro-1(2H)-pyrazinecarboxylate (342 mg, 0.880 mmol). The title compound was isolated as a beige solid. (287 mg, 76% yield). LCMS: [M+H]⁺=537.33

### Step 2: 6-(3-amino-5-fluoro-6-(4-(piperazin-1-yl)phenyl)pyrazin-2-yl)-7-fluoro-3,4-dihydroisoquinolin-1(2H)-one TFA salt

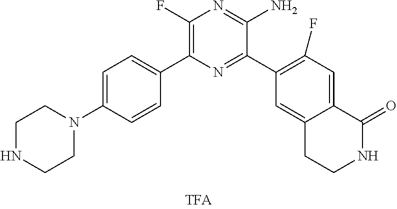 [SMILES: Nc1nc(F)c(-c2ccc(N3CCNCC3)cc2)nc1-c1cc2c(cc1F)C(=O)NCC2]

Prepared from tert-butyl 4-(4-(5-amino-3-fluoro-6-(7-fluoro-1-oxo-1,2,3,4-tetrahydroisoquinolin-6-yl)pyrazin-2-yl)phenyl)piperazine-1-carboxylate (135 mg, 0.252 mmol) and TFA (0.578 mL, 7.55 mmol) to give the product as a pale-yellow solid. (130 mg, 94% yield); LCMS: [M+H]⁺=437.31

### Step 3: 6-(3-amino-6-(4-(4-(4,4-difluorobutyl)piperazin-1-yl)phenyl)-5-fluoropyrazin-2-yl)-7-fluoro-3,4-dihydroisoquinolin-1(2H)-one

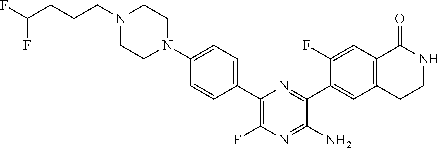 [SMILES: Nc1nc(F)c(-c2ccc(N3CCN(CCCC(F)F)CC3)cc2)nc1-c1cc2c(cc1F)C(=O)NCC2]

Prepared from 6-(3-amino-5-fluoro-6-(4-(piperazin-1-yl)phenyl)pyrazin-2-yl)-7-fluoro-3,4-dihydroisoquinolin-1(2H)-one-TFA (50 mg, 0.091 mmol) and 4-bromo-1,1-difluorobutane (31.4 mg, 0.182 mmol) to give the product as a pale-yellow solid (45 mg, 89% yield). ¹H NMR (500 MHz, METHANOL-d₄) δ ppm 7.70 (d, J=7.95 Hz, 2H), 7.66 (d, J=10.15 Hz, 1H), 7.42 (d, J=6.60 Hz, 1H), 6.89-6.96 (m, 2H), 5.71-5.98 (m, 1H), 3.47-3.48 (m, 1H), 3.44-3.48 (m, 2H), 3.17 (br. s., 4H), 2.94 (t, J=6.54 Hz, 2H), 2.53-2.63 (m, 4H), 2.37-2.43 (m, 2H), 1.73-1.84 (m, 2H), 1.59-1.66 (m, 2H); LCMS: [M+H]⁺=529.44.

### Example 70: 6-(3-amino-6-(4-(4-(4,4-difluorobutyl)piperazin-1-yl)phenyl)-5-fluoropyrazin-2-yl)isoquinolin-1(2H)-one (I-70)

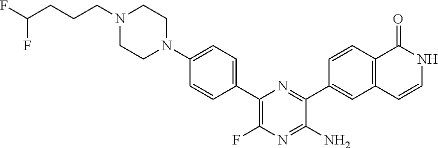 [SMILES: Nc1nc(F)c(-c2ccc(N3CCN(CCCC(F)F)CC3)cc2)nc1-c1ccc2c(=O)[nH]ccc2c1]

### Step 1: 6-(4,4,5,5-tetramethyl-1,3,2-dioxaborolan-2-yl)isoquinolin-1(2H)-one

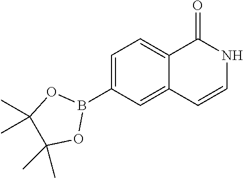 [SMILES: CC1(C)OB(c2ccc3c(=O)[nH]ccc3c2)OC1(C)C]

To a solution of 6-bromoisoquinolin-1(2H)-one (9 g, 40.16 mmol) in 1,4-dioxane (90 mL), potassium acetate (21.43 g, 120.48 mmol) and bis(pinacolato)diboron (5.6 g, 40.16 mmol) were added and the reaction was degassed using nitrogen for 10 min. Pd(dppf)Cl₂ (2.9 g, 4.01 mmol) was added under a nitrogen atmosphere and the mixture was stirred at 100° C. for 5 h. The reaction was diluted with EtOAc and filtered through celite. The volatiles were removed under reduced pressure to give the crude product (9 g, 47.62 mmol) which was used in the next step without further purification. LCMS [M+H]⁺=271.12 (pinacol ester) and [M+H]+ 188.92 (boronic acid).

### Step 2: 6-(2-amino-5-bromo-6-fluoropyridin-3-yl)isoquinolin-1(2H)-one

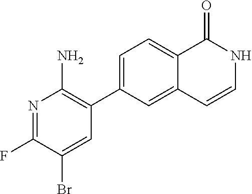 [SMILES: Nc1nc(F)c(Br)cc1-c1ccc2c(=O)[nH]ccc2c1]

To a solution of 6-(4,4,5,5-tetramethyl-1,3,2-dioxaborolan-2-yl)isoquinolin-1(2H)-one (12 g, 44.62 mmol) in dioxane (110 mL) and water (30 mL), sodium carbonate (13.95 g, 132.9 mmol) and 5-bromo-6-fluoro-3-iodopyridin-2-amine (14 g, 44.62 mmol) were added and the reaction mass was degassed using nitrogen for 10 min. To this, tetrakis (5.1 g, 4.4 mmol) was added under nitrogen atmosphere and reaction mass was stirred at 90° C. for 3 h. After completion of reaction, the reaction mass was diluted with water (100 mL) and extracted with EtOAc (3×100 mL). The combined organic layer was dried over anhydrous Na₂SO₄, concentrated under vacuum to get the crude. The crude material was purified by column chromatography and the product was eluted in 4% MeOH in DCM to afford the title compound (5.4 g, 16.16 mmol, 31.3%) as a light brown solid of 6-(2-amino-5-bromo-6-fluoropyridin-3-yl)isoquinolin-1(2H)-one. ¹H NMR (400 MHz, DMSO-d₆) δ 8.23 (d, J=8.3 Hz, 1H), 7.83-7.69 (m, 2H), 7.51 (d, J=8.7 Hz, 1H), 7.21 (d, J=7.1 Hz, 1H), 6.57 (d, J=7.2 Hz, 1H), 6.60 (d, J=7.2 Hz, 2H). LCMS [M+H]⁺=334.15.

### Step 3: 6-(2-amino-6-fluoro-5-(4-(piperazin-1-yl)phenyl)pyridin-3-yl)isoquinolin-1(2H)-one

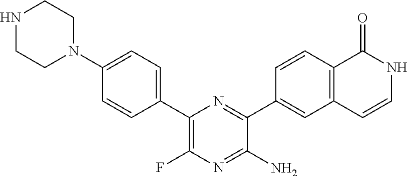 [SMILES: Nc1nc(F)c(-c2ccc(N3CCNCC3)cc2)nc1-c1ccc2c(=O)[nH]ccc2c1]

Prepared from 6-(2-amino-5-bromo-6-fluoropyridin-3-yl)isoquinolin-1(2H)-one (70 mg, 0.209 mmol) with 4-piperazinylphenylboronic acid, pinacol ester (78 mg, 0.272 mmol) to give the title compound as a beige solid. (65 mg, 75% yield); LCMS: [M+H]+=416.31.

### Step 4: 6-(3-amino-6-(4-(4-(4,4-difluorobutyl)piperazin-1-yl)phenyl)-5-fluoropyrazin-2-yl)isoquinolin-1(2H)-one

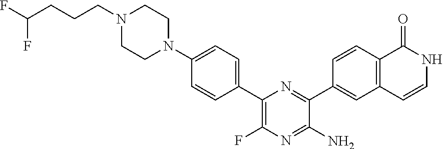 [SMILES: Nc1nc(F)c(-c2ccc(N3CCN(CCCC(F)F)CC3)cc2)nc1-c1ccc2c(=O)[nH]ccc2c1]

Prepared from 6-(3-amino-5-fluoro-6-(4-(piperazin-1-yl)phenyl)pyrazin-2-yl)isoquinolin-1(2H)-one (65 mg, 0.156 mmol) and 4-bromo-1,1-difluorobutane (54.0 mg, 0.312 mmol). The title product was isolated as a beige solid. (57 mg, 68% yield). ¹H NMR (500 MHz, METHANOL-d4) δ ppm 8.42 (d, J=8.31 Hz, 1H), 7.82 (s, 1H), 7.65-7.73 (m, 2H), 7.46 (d, J=7.95 Hz, 2H), 7.24 (d, J=7.09 Hz, 1H), 7.05 (d, J=8.80 Hz, 2H), 6.76 (d, J=7.09 Hz, 1H), 5.82-6.09 (m, 1H), 3.24-3.29 (m, 4H), 2.65-2.72 (m, 4H), 2.47-2.53 (m, 2H), 1.85-1.96 (m, 2H), 1.73 (quin, J=7.70 Hz, 2H); LCMS: [M+H]⁺=509.45.

### Example 71: 6-(3-amino-5-fluoro-6-(4-(4-isopropylpiperazin-1-yl)phenyl)pyrazin-2-yl)-4-fluoro-3-methylisoquinolin-1(2H)-one (I-71)

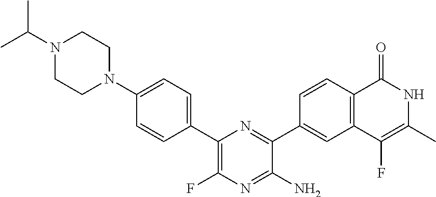 [SMILES: Cc1[nH]c(=O)c2ccc(-c3nc(-c4ccc(N5CCN(C(C)C)CC5)cc4)c(F)nc3N)cc2c1F]

### Step 1: 4-fluoro-3-methyl-6-(4,4,5,5-tetramethyl-1,3,2-dioxaborolan-2-yl)isoquinolin-1(2H)-one

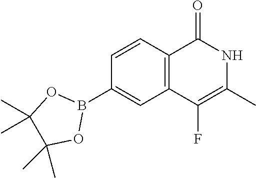

Prepared from 6-bromo-4-fluoro-3-methylisoquinolin-1(2H)-one (72 mg, 0.281 mmol) to give a mixture of boronate and boronic acid which was used in the next step without further purification. LCMS: [M+H]⁺=222.23 boronic acid, 304.29 boronate.

### Step 2: 6-(3-amino-5-fluoro-6-(4-(4-isopropylpiperazin-1-yl)phenyl)pyrazin-2-yl)-4-fluoro-3-methylisoquinolin-1(2H)-one

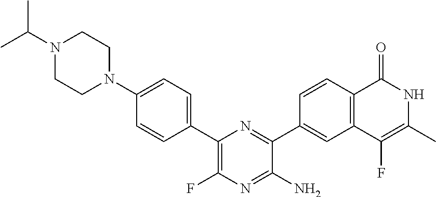 [SMILES: Cc1[nH]c(=O)c2ccc(-c3nc(-c4ccc(N5CCN(C(C)C)CC5)cc4)c(F)nc3N)cc2c1F]

Prepared from 4-fluoro-3-methyl-6-(4,4,5,5-tetramethyl-1,3,2-dioxaborolan-2-yl)isoquinolin-1(2H)-one (40.6 mg, 0.134 mmol) and 3-bromo-6-fluoro-5-(4-(4-isopropylpiperazin-1-yl)phenyl)pyrazin-2-amine (48 mg, 0.122 mmol) to give the product (5.9 mg, 9.9% yield) as a bright yellow solid. ¹H NMR (500 MHz, DMSO-d₆) δ ppm 11.29 (br. s., 1H), 8.26 (dd, J=8.25, 1.41 Hz, 1H), 7.99 (s, 1H), 7.87 (d, J=8.31 Hz, 1H), 7.76 (d, J=8.19 Hz, 2H), 7.03 (d, J=7.83 Hz, 2H), 6.83 (br. s., 2H), 3.18 (br. s., 4H), 2.67-2.73 (m, 1H), 2.59 (br. s., 4H), 2.25 (d, J=2.81 Hz, 3H), 0.97-1.08 (m, 6H); LCMS [M+H]⁺=491.34.

### Example 72: 6-(3-amino-6-(4-(4-(cyclopropylmethyl)piperazin-1-yl)phenyl)-5-fluoropyrazin-2-yl)-4-methylisoquinolin-1(2H)-one (I-72)

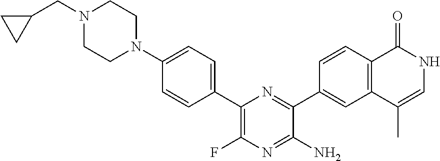 [SMILES: Cc1c[nH]c(=O)c2ccc(-c3nc(-c4ccc(N5CCN(CC6CC6)CC5)cc4)c(F)nc3N)cc12]

### Step 1: 6-(3-amino-6-bromo-5-fluoropyrazin-2-yl)-4-methylisoquinolin-1(2H)-one

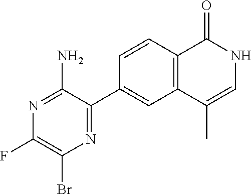 [SMILES: Cc1c[nH]c(=O)c2ccc(-c3nc(Br)c(F)nc3N)cc12]

Prepared from 5-bromo-6-fluoro-3-iodopyrazin-2-amine (405 mg, 1.274 mmol) and 4-methyl-6-(4,4,5,5-tetramethyl-1,3,2-dioxaborolan-2-yl)isoquinolin-1(2H)-one (436 mg, 1.529 mmol) to give 6-(3-amino-6-bromo-5-fluoropyrazin-2-yl)-4-methylisoquinolin-1(2H)-one (435 mg, 1.246 mmol, 98% yield) as a yellow solid. ¹H NMR (500 MHz, DMSO-d₆) δ ppm 11.15 (d, J=4.89 Hz, 1H), 8.31 (d, J=8.19 Hz, 1H), 7.89 (s, 1H), 7.70 (dd, J=8.38, 1.04 Hz, 1H), 7.02-7.14 (m, 3H), 2.24 (s, 3H); LCMS: [M+H]⁺=349.12.

### Step 2: 6-(3-amino-6-(4-(4-(cyclopropylmethyl)piperazin-1-yl)phenyl)-5-fluoropyrazin-2-yl)-4-methylisoquinolin-1(2H)-one

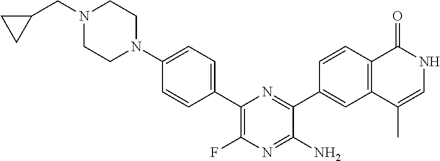 [SMILES: Cc1c[nH]c(=O)c2ccc(-c3nc(-c4ccc(N5CCN(CC6CC6)CC5)cc4)c(F)nc3N)cc12]

Prepared from 6-(3-amino-6-bromo-5-fluoropyrazin-2-yl)-4-methylisoquinolin-1(2H)-one (40 mg, 0.115 mmol) and 1-(cyclopropylmethyl)-4-(4-(4,4,5,5-tetramethyl-1,3,2-dioxaborolan-2-yl)phenyl)piperazine (47.1 mg, 0.137 mmol) to give the title compound (22.8 mg, 41.1% yield) as a yellow solid. ¹H NMR (500 MHz, DMSO-d₆) δ ppm 11.13 (br. s., 1H), 8.33 (d, J=8.31 Hz, 1H), 7.99 (s, 1H), 7.84 (d, J=8.31 Hz, 1H), 7.76 (d, J=8.19 Hz, 2H), 7.07 (br. s., 1H), 7.02 (d, J=8.93 Hz, 2H), 6.81 (s, 2H), 3.19-3.24 (m, 4H), 2.56-2.60 (m, 4H), 2.26 (s, 3H), 2.23 (d, J=6.48 Hz, 2H), 0.82-0.91 (m, 1H), 0.46-0.50 (m, 2H), 0.10 (q, J=4.89 Hz, 2H); LCMS [M+H]⁺485.32.

### Example 73: 6-(3-amino-6-(4-(4-(4,4-difluorobutyl)piperazin-1-yl)phenyl)-5-fluoropyrazin-2-yl)-4-methylisoquinolin-1(2H)-one (I-73)

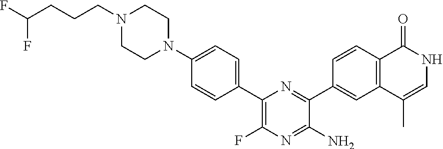 [SMILES: Cc1c[nH]c(=O)c2ccc(-c3nc(-c4ccc(N5CCN(CCCC(F)F)CC5)cc4)c(F)nc3N)cc12]

### Step 1: 6-(3-amino-5-fluoro-6-(4-(piperazin-1-yl)phenyl)pyrazin-2-yl)-4-methylisoquinolin-1(2H)-one

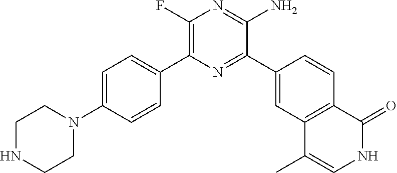 [SMILES: Cc1c[nH]c(=O)c2ccc(-c3nc(-c4ccc(N5CCNCC5)cc4)c(F)nc3N)cc12]

Prepared from 6-(3-amino-6-bromo-5-fluoropyrazin-2-yl)-4-methylisoquinolin-1(2H)-one (60 mg, 0.172 mmol) with 4-piperazinylphenylboronic acid, pinacol ester (64.4 mg, 0.223 mmol) to give the title compound as a beige solid (38 mg, 51% yield); LCMS: [M+H]⁺=431.36

### Step 2: 6-(3-amino-6-(4-(4-(4,4-difluorobutyl)piperazin-1-yl)phenyl)-5-fluoropyrazin-2-yl)-4-methylisoquinolin-1(2H)-one

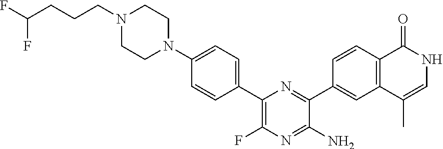 [SMILES: Cc1c[nH]c(=O)c2ccc(-c3nc(-c4ccc(N5CCN(CCCC(F)F)CC5)cc4)c(F)nc3N)cc12]

Prepared from 6-(3-amino-5-fluoro-6-(4-(piperazin-1-yl)phenyl)pyrazin-2-yl)-4-methylisoquinolin-1(2H)-one (38 mg, 0.088 mmol) and 4-bromo-1,1-difluorobutane (30.5 mg, 0.177 mmol) to give the title product was isolated as a beige solid. (30 mg, 62% yield). ¹H NMR (500 MHz, METHANOL-d₄) δ ppm 8.49 (d, J=8.31 Hz, 1H), 8.17 (s, 1H), 7.97 (d, J=8.31 Hz, 1H), 7.89 (d, J=8.31 Hz, 2H), 7.04-7.14 (m, 3H), 5.84-6.08 (m, 1H), 3.26-3.31 (m, 4H), 2.65-2.73 (m, 4H), 2.50 (t, J=7.52 Hz, 2H), 2.39 (s, 3H), 1.85-1.97 (m, 2H), 1.73 (quin, J=7.58 Hz, 2H); LCMS: [M+H]⁺=523.49.

### Example 74: 7-(3-amino-5-fluoro-6-(4-((1S,5R)-3-methyl-3-azabicyclo[3.1.0]hexan-1-yl)phenyl)pyrazin-2-yl)-2-methylquinazolin-4(3H)-one (I-74)

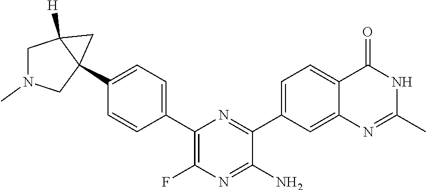

Prepared from 7-(3-amino-6-bromo-5-fluoropyrazin-2-yl)-2-methylquinazolin-4(3H)-one (40 mg, 0.114 mmol) reacted with (1 S,5R)-3-methyl-1-(4-(4,4,5,5-tetramethyl-1,3,2-dioxaborolan-2-yl)phenyl)-3-azabicyclo[3.1.0]hexane (43.1 mg, 0.114 mmol, prepared from the corresponding bromide using standard borylation conditions) to give the title compound (23.2 mg, 45.7% yield). ¹H NMR (500 MHz, DMSO-d₆) δ=12.26 (s, 1H), 8.17 (d, J=8.3 Hz, 1H), 7.90 (s, 1H), 7.84-7.74 (m, 3H), 7.26 (br d, J=7.5 Hz, 2H), 6.95 (br s, 2H), 3.27-3.00 (m, 1H), 2.85-2.61 (m, 1H), 2.50-2.23 (m, 8H), 1.98-1.83 (m, 1H), 1.36 (t, J=4.2 Hz, 1H), 0.98-0.82 (m, 1H); LCMS: [M+H]⁺=443.39.

### Example 75: 6-(3-amino-5-fluoro-6-(4-((1S,5R)-3-methyl-3-azabicyclo[3.1.0]hexan-1-yl)phenyl)pyrazin-2-yl)-4-methylisoquinolin-1(2H)-one (I-75)

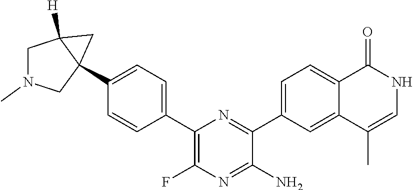 [SMILES: Cc1c[nH]c(=O)c2ccc(-c3nc(-c4ccc([C@]56C[C@H]5CN(C)C6)cc4)c(F)nc3N)cc12]

Prepared from 6-(3-amino-6-bromo-5-fluoropyrazin-2-yl)-4-methylisoquinolin-1(2H)-one (40 mg, 0.115 mmol) and (1S,5R)-3-methyl-1-(4-(4,4,5,5-tetramethyl-1,3,2-dioxaborolan-2-yl)phenyl)-3-azabicyclo[3.1.0]hexane (43.2 mg, 0.115 mmol, prepared from the corresponding bromide using methods similar to previous Examples) to give the title compound (19.3 mg, 37.5% yield). ¹H NMR (500 MHz, DMSO-d₆) b=11.14 (br d, J=5.3 Hz, 1H), 8.34 (d, J=8.3 Hz, 1H), 7.99 (s, 1H), 7.83 (br dd, J=8.7, 12.2 Hz, 3H), 7.26 (brd, J=8.1 Hz, 2H), 7.08 (br d, J=5.6 Hz, 1H), 6.97 (s, 2H), 3.38 (br d, J=5.5 Hz, 1H), 3.09 (br d, J=5.6 Hz, 1H), 2.82-2.67 (m, 1H), 2.66-2.55 (m, 1H), 2.39 (br s, 3H), 2.25 (s, 3H), 1.93-1.84 (m, 1H), 1.34 (brt, J=4.2 Hz, 1H), 0.93-0.82 (m, 1H); LCMS: [M+H]⁺=442.45.

### Example 76: 7-(3-amino-6-(4-(4-cyclobutylpiperazin-1-yl)phenyl)-5-fluoropyrazin-2-yl)-2-methylquinazolin-4(3H)-one (I-76)

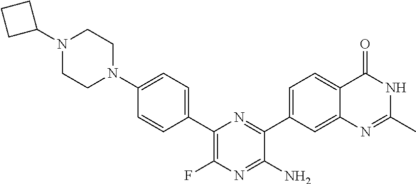 [SMILES: Cc1nc2cc(-c3nc(-c4ccc(N5CCN(C6CCC6)CC5)cc4)c(F)nc3N)ccc2c(=O)[nH]1]

### Step 1: 7-(3-Amino-6-bromo-5-fluoropyrazin-2-yl)-2-methylquinazolin-4(3H)-one

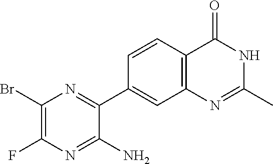 [SMILES: Cc1nc2cc(-c3nc(Br)c(F)nc3N)ccc2c(=O)[nH]1]

7-(3-Amino-6-bromo-5-fluoropyrazin-2-yl)-2-methylquinazolin-4(3H)-one (1.404 g, 53.2% yield) was prepared from 7-bromo-2-methylquinazolin-4(3H)-one (1.8 g, 7.53 mmol) reacted with potassium acetate (2.217 g, 22.59 mmol) 1,1′-bis(diphenylphosphino)ferrocene-palladium(II)dichloride (0.552 g, 0.753 mmol) and bis(pinacolato)diboron (2.103 g, 8.28 mmol) were mixed in 1,4-dioxane (50 ml) under vacuum and stirred under at 90° C. oil bath overnight, followed by being reacted with 5-bromo-6-fluoro-3-iodopyrazin-2-amine (2.87 g, 9.03 mmol) bis(triphenylphosphine)palladium(II) dichloride (0.634 g, 0.903 mmol) and sodium carbonate monohydrate (2.80 g, 22.59 mmol) in acetonitrile (50.0 ml) and water (20 ml) under vacuum and stirred under 85° C. oil bath overnight. LCMS: [M+H]⁺=350.25

### Step 2: 7-(3-amino-6-(4-(4-cyclobutylpiperazin-1-yl)phenyl)-5-fluoropyrazin-2-yl)-2-methylquinazolin-4(3H)-one

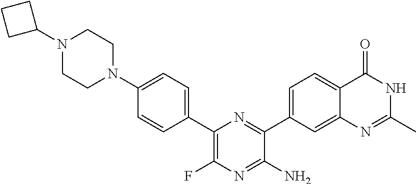 [SMILES: Cc1nc2cc(-c3nc(-c4ccc(N5CCN(C6CCC6)CC5)cc4)c(F)nc3N)ccc2c(=O)[nH]1]

Prepared from 7-(3-amino-6-bromo-5-fluoropyrazin-2-yl)-2-methylquinazolin-4(3H)-one (40 mg, 0.114 mmol) and 1-cyclobutyl-4-(4-(4,4,5,5-tetramethyl-1,3,2-dioxaborolan-2-yl)phenyl)piperazine (39.1 mg, 0.114 mmol, prepared from the corresponding bromide using standard borylation procedures) to give the title compound (29.2 mg, 51.3% yield). ¹H NMR (500 MHz, DMSO-d₆) δ=12.25 (br d, J=1.3 Hz, 1H), 8.17 (d, J=8.1 Hz, 1H), 7.91 (s, 1H), 7.83-7.72 (m, 3H), 7.02 (br d, J=8.8 Hz, 2H), 6.79 (s, 2H), 3.58 (s, 1H), 3.36 (s, 1H), 3.20 (br d, J=4.4 Hz, 4H), 2.74 (quin, J=7.6 Hz, 1H), 2.39 (s, 7H), 2.05-1.94 (m, 2H), 1.89-1.76 (m, 2H), 1.71-1.61 (m, 2H); LCMS: [M+H]⁺=486.39.

### Example 77: 7-(3-amino-6-(3-((dimethylamino)methyl)-4-morpholinophenyl)-5-fluoropyrazin-2-yl)-2-methylquinazolin-4(3H)-one (I-77)

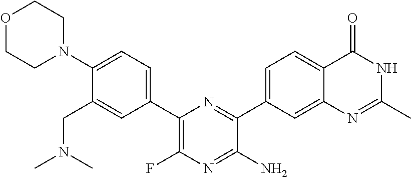 [SMILES: Cc1nc2cc(-c3nc(-c4ccc(N5CCOCC5)c(CN(C)C)c4)c(F)nc3N)ccc2c(=O)[nH]1]

Prepared from 7-(3-amino-6-bromo-5-fluoropyrazin-2-yl)-2-methylquinazolin-4(3H)-one (40 mg, 0.114 mmol) and N,N-dimethyl-1-(2-morpholino-5-(4,4,5,5-tetramethyl-1,3,2-dioxaborolan-2-yl)phenyl)methanamine (39.6 mg, 0.114 mmol) to give the title compound (12.7 mg, 21.2% yield). ¹H NMR (500 MHz, DMSO-d₆) δ=8.18 (d, J=8.2 Hz, 1H), 7.91 (s, 1H), 7.87 (s, 1H), 7.84-7.74 (m, 2H), 7.32-7.24 (m, 1H), 3.75 (br s, 6H), 2.96-2.86 (m, 4H), 2.45-2.29 (m, 9H); LCMS: [M+H]⁺=490.46.

### Example 78: 6-(3-amino-5-fluoro-6-(4-(4-isopropylpiperazin-1-yl)phenyl)pyrazin-2-yl)-4-chloroisoquinolin-1(2H)-one (I-78)

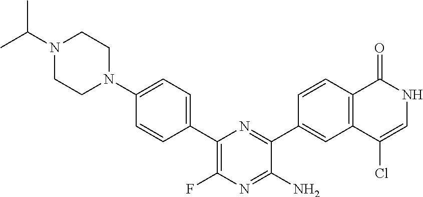 [SMILES: CC(C)N1CCN(c2ccc(-c3nc(-c4ccc5c(=O)[nH]cc(Cl)c5c4)c(N)nc3F)cc2)CC1]

### Step 1: 6-bromo-4-chloroisoquinolin-1(2H)-one

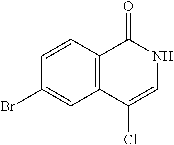 [SMILES: O=c1[nH]cc(Cl)c2cc(Br)ccc12]

To a solution of 6-bromo-2H-isoquinolin-1-one (600 mg, 2.68 mmol) in N,N-dimethylacetamide (14 ml) was added N-chlorosuccinimide (429 mg, 3.21 mmol). The mixture was stirred at 50° C. for 1 hour then cooled to RT and diluted with water. The white suspension was filtered, washed with water, and dried under vacuum to obtain 6-bromo-4-chloroisoquinolin-1(2H)-one (615 mg, 1.927 mmol, 72.0% yield, Purity (UV 254) 81%) as a white solid. The material was used in the next step without further purification. LCMS: [M+H]⁺=258.15.

### Step 2: 4-chloro-6-(4,4,5,5-tetramethyl-1,3,2-dioxaborolan-2-yl)isoquinolin-1(2H)-one

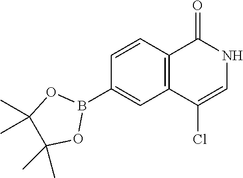 [SMILES: CC1(C)OB(c2ccc3c(=O)[nH]cc(Cl)c3c2)OC1(C)C]

Prepared from 6-bromo-4-chloroisoquinolin-1(2H)-one (132 mg, 0.511 mmol) to give a mixture of boronate and boronic acid which was used in the next step without further purification. LCMS[M+H]⁺306.24 Boronate, 224.17 Boronic acid.

### Step 3: 6-(3-amino-5-fluoro-6-(4-(4-isopropylpiperazin-1-yl)phenyl)pyrazin-2-yl)-4-chloroisoquinolin-1(2H)-one

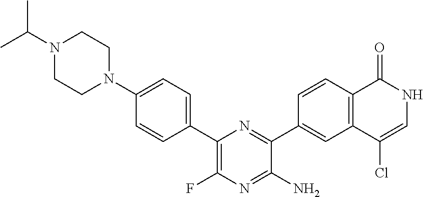 [SMILES: CC(C)N1CCN(c2ccc(-c3nc(-c4ccc5c(=O)[nH]cc(Cl)c5c4)c(N)nc3F)cc2)CC1]

Prepared from 4-chloro-6-(4,4,5,5-tetramethyl-1,3,2-dioxaborolan-2-yl)isoquinolin-1(2H)-one (74.4 mg, 0.110 mmol) and 3-bromo-6-fluoro-5-(4-(4-isopropylpiperazin-1-yl)phenyl)pyrazin-2-amine (36 mg, 0.091 mmol) to give the title compound (7.3 mg, 16.2% yield) as a yellow solid. ¹H NMR (500 MHz, DMSO-d₆) δ ppm 11.62 (d, J=5.01 Hz, 1H), 8.35 (d, J=8.31 Hz, 1H), 8.15 (s, 1H), 7.95 (d, J=8.31 Hz, 1H), 7.75 (d, J=8.44 Hz, 2H), 7.54 (d, J=5.62 Hz, 1H), 7.02 (d, J=8.80 Hz, 2H), 6.83 (s, 2H), 3.19 (br. s., 4H), 2.68 (br. s., 1H), 2.59 (br. s., 4H), 1.01 (d, J=6.36 Hz, 6H); LCMS: [M+H]⁺=493.47.

### Example 79: 7-(3-amino-6-(4-(4-(cyclopropylmethyl)piperazin-1-yl)phenyl)-5-fluoropyrazin-2-yl)-2-methylquinazolin-4(3H)-one (I-79)

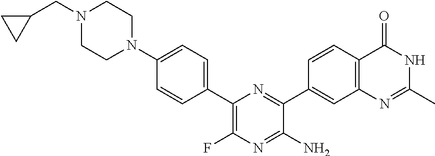 [SMILES: Cc1nc2cc(-c3nc(-c4ccc(N5CCN(CC6CC6)CC5)cc4)c(F)nc3N)ccc2c(=O)[nH]1]

Prepared from 7-(3-amino-6-bromo-5-fluoropyrazin-2-yl)-2-methylquinazolin-4(3H)-one (40 mg, 0.114 mmol) and 1-(cyclopropylmethyl)-4-(4-(4,4,5,5-tetramethyl-1,3,2-dioxaborolan-2-yl)phenyl)piperazine (46.9 mg, 0.137 mmol) to give the title compound (24.6 mg, 44.3% yield) as a yellow solid. ¹H NMR (500 MHz, DMSO-d₆) δ ppm 12.26 (br. s., 1H), 8.17 (d, J=8.19 Hz, 1H), 7.90 (s, 1H), 7.80 (d, J=8.31 Hz, 1H), 7.77 (d, J=8.44 Hz, 2H), 7.03 (d, J=8.68 Hz, 2H), 6.79 (s, 2H), 3.22 (br. s., 4H), 2.59 (br. s., 4H), 2.38 (s, 3H), 2.24 (br. s., 2H), 0.87 (br. s., 1H), 0.49 (d, J=7.21 Hz, 2H), 0.11 (br. s., 2H); LCMS: [M+H]⁺=486.51.

### Example 80: 6-(3-amino-5-fluoro-6-(4-((1S,5R)-3-methyl-3-azabicyclo[3.1.0]hexan-1-yl)phenyl)pyrazin-2-yl)-4-fluoroisoquinolin-1(2H)-one (I-80)

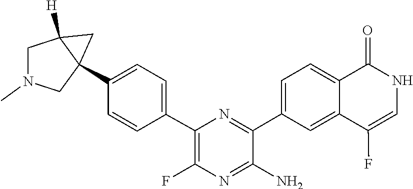 [SMILES: CN1C[C@@H]2C[C@]2(c2ccc(-c3nc(-c4ccc5c(=O)[nH]cc(F)c5c4)c(N)nc3F)cc2)C1]

Prepared from 6-(3-amino-6-bromo-5-fluoropyrazin-2-yl)-4-fluoroisoquinolin-1(2H)-one (40 mg, 0.113 mmol) and (1S,5R)-3-methyl-1-(4-(4,4,5,5-tetramethyl-1,3,2-dioxaborolan-2-yl)phenyl)-3-azabicyclo[3.1.0]hexane (42.7 mg, 0.113 mmol, prepared from borylation of the corresponding bromide) to give the title compound (13.6 mg, 26.2% yield). ¹H NMR (500 MHz, METHANOL-d₄) δ=8.44 (br d, J=8.3 Hz, 1H), 8.22 (s, 1H), 8.03 (br d, J=8.3 Hz, 1H), 7.91 (br d, J=7.9 Hz, 2H), 7.37-7.22 (m, 3H), 3.62 (br d, J=9.5 Hz, 1H), 3.37 (br d, J=10.0 Hz, 1H), 3.09 (br d, J=9.4 Hz, 1H), 3.01 (br dd, J=2.9, 9.7 Hz, 1H), 2.64 (s, 3H), 2.06-1.97 (m, 1H), 1.97-1.87 (m, 1H), 1.36 (br t, J=4.5 Hz, 1H), 1.11-1.03 (m, 1H); LCMS: [M+H]⁺=446.34.

### Example 81: 7-(3-amino-6-(4-(4-(4,4-difluorobutyl)piperazin-1-yl)phenyl)-5-fluoropyrazin-2-yl)-2-methylquinazolin-4(3H)-one (I-82)

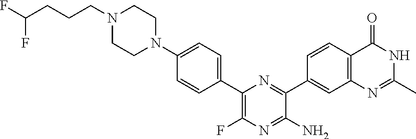 [SMILES: Cc1nc2cc(-c3nc(-c4ccc(N5CCN(CCCC(F)F)CC5)cc4)c(F)nc3N)ccc2c(=O)[nH]1]

### Step 1: 7-(3-amino-5-fluoro-6-(4-(piperazin-1-yl)phenyl)pyrazin-2-yl)-2-methylquinazolin-4(3H)-one

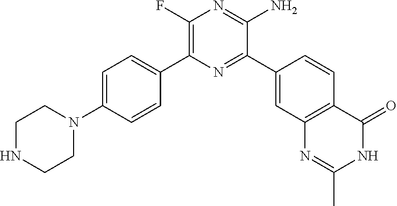 [SMILES: Cc1nc2cc(-c3nc(-c4ccc(N5CCNCC5)cc4)c(F)nc3N)ccc2c(=O)[nH]1]

Prepared from 7-(3-amino-6-bromo-5-fluoropyrazin-2-yl)-2-methylquinazolin-4(3H)-one (60 mg, 0.171 mmol) and 4-piperazinylphenylboronic acid, pinacol ester (64.2 mg, 0.223 mmol) commercially available. The reaction mixture was concentrated onto celite and purified by silica gel chromatography, eluting with CH₂Cl₂ containing 0-6% methanol and 0-0.6% NH₄OH to give the product as a yellow solid (51 mg, 51% yield); LCMS: [M+H]⁺=432.36

### Step 2: 7-(3-amino-6-(4-(4-(4,4-difluorobutyl)piperazin-1-yl)phenyl)-5-fluoropyrazin-2-yl)-2-methylquinazolin-4(3H)-one

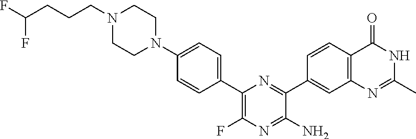 [SMILES: Cc1nc2cc(-c3nc(-c4ccc(N5CCN(CCCC(F)F)CC5)cc4)c(F)nc3N)ccc2c(=O)[nH]1]

Prepared from 7-(3-amino-5-fluoro-6-(4-(piperazin-1-yl)phenyl)pyrazin-2-yl)-2-methylquinazolin-4(3H)-one (51 mg, 0.118 mmol) Example 75, step 1 and 4-bromo-1,1-difluorobutane (40.9 mg, 0.236 mmol) to give the title compound as a pale-yellow solid. (45 mg, 69% yield). ¹H NMR (500 MHz, DMSO-d₆) δ ppm 12.26 (br. s., 1H), 8.14-8.22 (m, 1H), 7.91 (s, 1H), 7.75-7.85 (m, 3H), 7.03 (d, J=8.80 Hz, 2H), 6.80 (br. s., 2H), 6.01-6.29 (m, 1H), 3.21 (br. s., 4H), 2.53-2.62 (m, 4H), 2.35-2.40 (m, 5H), 1.81-1.92 (m, 2H), 1.59 (quin, J=7.27 Hz, 2H); LCMS: [M+H]⁺=524.49

### Example 82: 6-(3-amino-6-(4-(4-(3,3-difluoropropyl)piperazin-1-yl)phenyl)-5-fluoropyrazin-2-yl)-8-fluoro-3,4-dihydroisoquinolin-1(2H)-one (I-82)

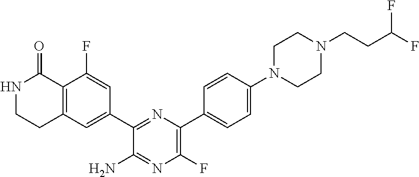 [SMILES: Nc1nc(F)c(-c2ccc(N3CCN(CCC(F)F)CC3)cc2)nc1-c1cc(F)c2c(c1)CCNC2=O]

### Step 1: 1-(3,3-difluoropropyl)-4-(4-(4,4,5,5-tetramethyl-1,3,2-dioxaborolan-2-yl)phenyl)piperazine

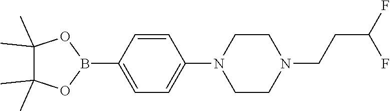 [SMILES: CC1(C)OB(c2ccc(N3CCN(CCC(F)F)CC3)cc2)OC1(C)C]

Prepared from 1-(4-bromophenyl)-4-(3,3-difluoropropyl)piperazine (225 mg, 0.705 mmol) and the resulting black solid was taken to the next step without any purification. (245 mg, 95% yield); LCMS: [M+H]⁺=367.42

### Step 2: 6-(3-amino-6-(4-(4-(3,3-difluoropropyl)piperazin-1-yl)phenyl)-5-fluoropyrazin-2-yl)-8-fluoro-3,4-dihydroisoquinolin-1(2H)-one

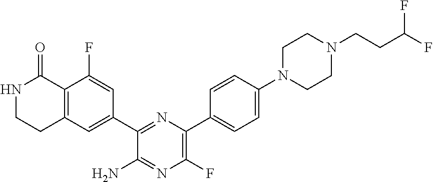 [SMILES: Nc1nc(F)c(-c2ccc(N3CCN(CCC(F)F)CC3)cc2)nc1-c1cc(F)c2c(c1)CCNC2=O]

Prepared from 6-(3-amino-6-bromo-5-fluoropyrazin-2-yl)-8-fluoro-3,4-dihydroisoquinolin-1(2H)-one (50 mg, 0.141 mmol) and 1-(3,3-difluoropropyl)-4-(4-(4,4,5,5-tetramethyl-1,3,2-dioxaborolan-2-yl)phenyl)piperazine (61.9 mg, 0.169 mmol) to give the title compound as a beige solid. (15 mg, 19% yield). ¹H NMR (500 MHz, DMSO-d₆) b=8.08-7.98 (m, 1H), 7.82-7.68 (m, 2H), 7.54-7.48 (m, 1H), 7.43 (br d, J=12.1 Hz, 1H), 7.11-6.97 (m, 2H), 6.87-6.70 (m, 2H), 6.27-6.02 (m, 1H), 3.42-3.39 (m, 2H), 3.22 (br s, 4H), 3.03-2.97 (m, 2H), 2.60-2.55 (m, 4H), 2.45-2.41 (m, 2H), 2.14-1.99 (m, 2H); LCMS: [M+H]⁺=515.48.

### Example 83: 6-(3-amino-6-(3-((dimethylamino)methyl)-4-(tetrahydro-2H-pyran-4-yl)phenyl)-5-fluoropyrazin-2-yl)-8-fluoro-3,4-dihydroisoquinolin-1(2H)-one (I-83)

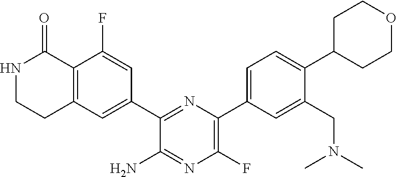 [SMILES: CN(C)Cc1cc(-c2nc(-c3cc(F)c4c(c3)CCNC4=O)c(N)nc2F)ccc1C1CCOCC1]

Prepared from 6-(3-amino-6-bromo-5-fluoropyrazin-2-yl)-8-fluoro-3,4-dihydroisoquinolin-1(2H)-one (44.4 mg, 0.125 mmol) and N,N-dimethyl-1-(2-(tetrahydro-2H-pyran-4-yl)-5-(4,4,5,5-tetramethyl-1,3,2-dioxaborolan-2-yl)phenyl)methanamine (54 mg, 0.156 mmol) to give the title compound as a beige solid (13, 20% yield). ¹H NMR (500 MHz, METHANOL-d₄) δ ppm 7.79-7.92 (m, 2H), 7.79-7.92 (m, 2H), 7.59 (s, 1H), 7.52 (d, J=11.98 Hz, 1H), 7.43 (d, J=8.07 Hz, 1H), 4.08 (dd, J=11.13, 3.55 Hz, 2H), 3.57-3.68 (m, 4H), 3.52 (t, J=6.42 Hz, 2H), 3.26-3.31 (m, 1H), 3.09 (t, J=6.36 Hz, 2H), 2.24-2.41 (m, 6H), 1.88 (qd, J=12.41, 4.10 Hz, 2H), 1.74 (d, J=12.59 Hz, 2H); LCMS: [M+H]⁺=494.53.

### Example 84: 6-(3-amino-6-(4-(4-(cyclopropylmethyl)piperazin-1-yl)phenyl)-5-fluoropyrazin-2-yl)-4-fluoroisoquinolin-1(2H)-one (I-84)

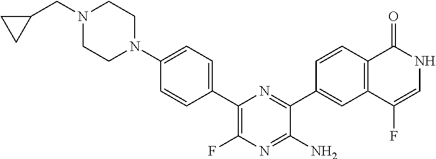 [SMILES: Nc1nc(F)c(-c2ccc(N3CCN(CC4CC4)CC3)cc2)nc1-c1ccc2c(=O)[nH]cc(F)c2c1]

Prepared from 6-(3-amino-6-bromo-5-fluoropyrazin-2-yl)-4-fluoroisoquinolin-1(2H)-one (36 mg, 0.102 mmol) and 1-(cyclopropylmethyl)-4-(4-(4,4,5,5-tetramethyl-1,3,2-dioxaborolan-2-yl)phenyl)piperazine (41.9 mg, 0.122 mmol) to give the title compound (17.9 mg, 35.9% yield) as a yellow solid. ¹H NMR (500 MHz, DMSO-d₆) δ ppm 11.20 (br. s., 1H), 8.29-8.34 (m, 1H), 8.06 (s, 1H), 7.95 (d, J=8.31 Hz, 1H), 7.76 (d, J=8.19 Hz, 2H), 7.44 (d, J=5.87 Hz, 1H), 7.03 (d, J=8.80 Hz, 2H), 6.86 (s, 2H), 3.19-3.24 (m, 4H), 2.56-2.60 (m, 4H), 2.22 (d, J=6.60 Hz, 2H), 0.82-0.91 (m, 1H), 0.45-0.50 (m, 2H), 0.10 (q, J=4.93 Hz, 2H); LCMS [M+H]⁺489.39.

### Example 85: 6-(3-amino-5-fluoro-6-(4-(4-isopropylpiperazin-1-yl)phenyl)pyrazin-2-yl)-4,8-difluoroisoquinolin-1(2H)-one (I-85)

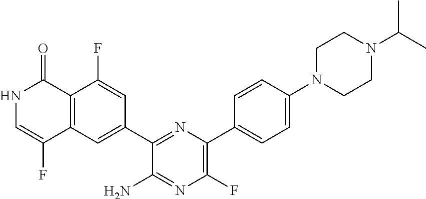 [SMILES: CC(C)N1CCN(c2ccc(-c3nc(-c4cc(F)c5c(=O)[nH]cc(F)c5c4)c(N)nc3F)cc2)CC1]

### Step 1: 6-bromo-4,8-difluoroisoquinolin-1(2H)-one

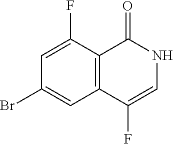 [SMILES: O=c1[nH]cc(F)c2cc(Br)cc(F)c12]

A vial was charged with 6-bromo-8-fluoro-1,2-dihydroisoquinolin-1-one (300 mg, 1.239 mmol) and Selectfluor™ fluorinating reagent >95% in F+ active (527 mg, 1.487 mmol). Methanol (3 ml) and acetonitrile (3 ml) were added, and the reaction was heated at 50° C. for 1 h. The reaction was concentrated in vacuo, dissolved in 1,2-dichloroethane (6 ml), and phosphorous (V) oxychloride (0.232 ml, 2.479 mmol) was added. The reaction was stirred at 50° C. for 1 h. The reaction was quenched into ice water and extracted with DCM (2×). The combined organic layers were dried with anhydrous sodium sulfate and concentrated to afford 6-bromo-4,8-difluoro-3-methoxy-3,4-dihydroisoquinolin-1(2H)-one (306 mg, 1.177 mmol, 95%) as a beige solid. The material was used in the next step as-is. LCMS: [M+H]⁺=260.16.

### Step 2: (4,8-difluoro-1-oxo-1,2-dihydroisoquinolin-6-yl)boronic acid

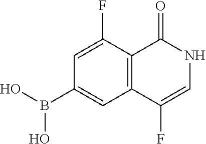 [SMILES: O=c1[nH]cc(F)c2cc(B(O)O)cc(F)c12]

Prepared from 6-bromo-4,8-difluoroisoquinolin-1(2H)-one (120 mg, 0.461 mmol) to give the boronic acid which was used in the next step without further purification. LCMS: [M+H]+ 226.11.

### Step 3: 6-(3-amino-5-fluoro-6-(4-(4-isopropylpiperazin-1-yl)phenyl)pyrazin-2-yl)-4,8-difluoroisoquinolin-1(2H)-one

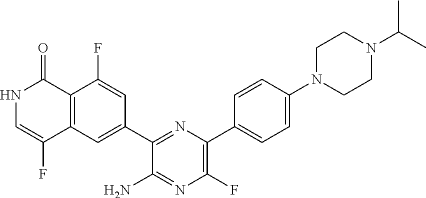 [SMILES: CC(C)N1CCN(c2ccc(-c3nc(-c4cc(F)c5c(=O)[nH]cc(F)c5c4)c(N)nc3F)cc2)CC1]

Prepared from (4,8-difluoro-1-oxo-1,2-dihydroisoquinolin-6-yl)boronic acid (33.4 mg, 0.148 mmol) and 3-bromo-6-fluoro-5-(4-(4-isopropylpiperazin-1-yl)phenyl)pyrazin-2-amine (45 mg, 0.114 mmol) to give the title compound (22 mg, 38.9% yield) as a yellow solid. ¹H NMR (500 MHz, DMSO-d₆) δ ppm 11.20 (br. s., 1H), 7.84 (s, 1H), 7.75 (d, J=8.07 Hz, 2H), 7.65 (d, J=12.10 Hz, 1H), 7.49 (br. s., 1H), 7.02 (d, J=8.93 Hz, 2H), 6.93 (s, 2H), 3.18-3.20 (m, 4H), 2.68 (d, J=6.36 Hz, 1H), 2.57-2.59 (m, 4H), 1.01 (d, J=6.48 Hz, 6H); LCMS: [M+H]⁺=495.35.

### Example 86: 6-(3-amino-6-(4-(4-(3,3-difluoropropyl)piperazin-1-yl)phenyl)-5-fluoropyrazin-2-yl)isoquinolin-1(2H)-one (I-86)

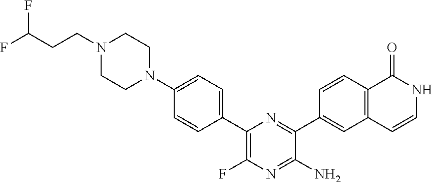 [SMILES: Nc1nc(F)c(-c2ccc(N3CCN(CCC(F)F)CC3)cc2)nc1-c1ccc2c(=O)[nH]ccc2c1]

Prepared from and 1-(3,3-difluoropropyl)-4-(4-(4,4,5,5-tetramethyl-1,3,2-dioxaborolan-2-yl)phenyl)piperazine (65.6 mg, 0.179 mmol) to give the title compound as a pale-yellow solid. (4.5 mg, 6% yield). ¹H NMR (500 MHz, METHANOL-d₄) δ ppm 8.33 (d, J=8.31 Hz, 1H), 7.97 (s, 1H), 7.83 (d, J=8.44 Hz, 1H), 7.77 (d, J=8.56 Hz, 2H), 7.11-7.16 (m, 1H), 6.95 (d, J=8.80 Hz, 2H), 6.67 (d, J=7.09 Hz, 1H), 5.77-6.02 (m, 1H), 3.18 (br. s., 4H), 2.56-2.62 (m, 4H), 2.50 (d, J=7.83 Hz, 2H), 1.96-2.06 (m, 2H); LCMS: [M+H]⁺=495.35.

### Example 87: 6-(3-amino-6-(4-(4-(3,3-difluoropropyl)piperazin-1-yl)phenyl)-5-fluoropyrazin-2-yl)-4-methylisoquinolin-1(2H)-one (I-87)

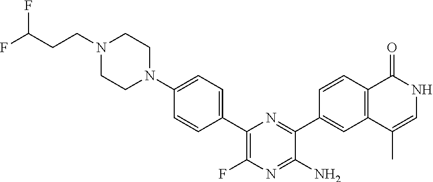 [SMILES: Cc1c[nH]c(=O)c2ccc(-c3nc(-c4ccc(N5CCN(CCC(F)F)CC5)cc4)c(F)nc3N)cc12]

Prepared from 6-(3-amino-6-bromo-5-fluoropyrazin-2-yl)-4-methylisoquinolin-1(2H)-one (50 mg, 0.143 mmol) and 1-(3,3-difluoropropyl)-4-(4-(4,4,5,5-tetramethyl-1,3,2-dioxaborolan-2-yl)phenyl)piperazine (62.9 mg, 0.172 mmol) to give the title compound as a pale-yellow solid. (10 mg, 13% yield). ¹H NMR (500 MHz, METHANOL-d₄) b ppm 8.37 (d, J=8.31 Hz, 1H), 8.05 (s, 1H), 7.85 (d, J=8.31 Hz, 1H), 7.77 (d, J=8.44 Hz, 2H), 6.94-7.00 (m, 3H), 5.79-6.04 (m, 1H), 3.10-3.19 (m, 4H), 2.58-2.64 (m, 4H), 2.52 (d, J=7.82 Hz, 2H), 2.26-2.30 (m, 3H), 1.98-2.08 (m, 2H); LCMS: [M+H]⁺=509.45.

### Example 88: 6-(3-amino-6-(3-((dimethylamino)methyl)-4-morpholinophenyl)-5-fluoropyrazin-2-yl)-8-fluoro-3,4-dihydroisoquinolin-1(2H)-one (I-88)

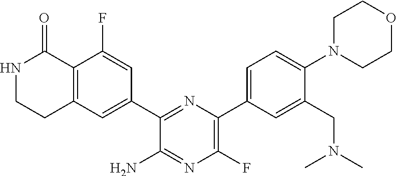 [SMILES: CN(C)Cc1cc(-c2nc(-c3cc(F)c4c(c3)CCNC4=O)c(N)nc2F)ccc1N1CCOCC1]

Prepared from 6-(3-amino-6-bromo-5-fluoropyrazin-2-yl)-8-fluoro-3,4-dihydroisoquinolin-1(2H)-one (50 mg, 0.141 mmol) and N,N-dimethyl-1-(2-morpholino-5-(4,4,5,5-tetramethyl-1,3-dioxolan-2-yl)phenyl)methanamine (63.8 mg, 0.183 mmol) to give the title compound as a yellow solid (37 mg, 50.5% yield). ¹H NMR (500 MHz, DMSO-d₆) b ppm 8.05 (br. s., 1H), 7.87 (br. s., 1H), 7.74 (d, J=8.07 Hz, 1H), 7.49 (s, 1H), 7.42 (d, J=11.74 Hz, 1H), 7.18 (d, J=8.44 Hz, 1H), 6.94 (br. s., 2H), 3.76 (br. s., 4H), 3.47 (br. s., 2H), 3.36-3.40 (m, 2H), 2.96 (br. s., 6H), 2.20 (s, 6H); LCMS: [M+H]+=495.47.

### Example 89: 6-(3-amino-6-(3-((dimethylamino)methyl)-4-morpholinophenyl)-5-fluoropyrazin-2-yl)-4-fluoroisoquinolin-1(2H)-one (I-89)

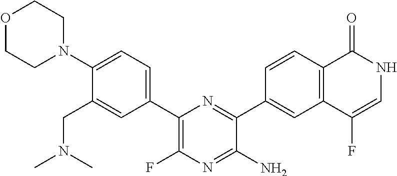 [SMILES: CN(C)Cc1cc(-c2nc(-c3ccc4c(=O)[nH]cc(F)c4c3)c(N)nc2F)ccc1N1CCOCC1]

### Step 1: N,N-dimethyl-1-(2-morpholino-5-(4,4,5,5-tetramethyl-1,3,2-dioxaborolan-2-yl)phenyl)methanamine

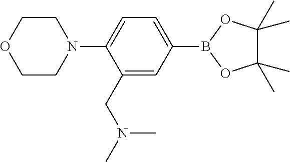 [SMILES: CN(C)Cc1cc(B2OC(C)(C)C(C)(C)O2)ccc1N1CCOCC1]

Prepared from 1-(5-bromo-2-morpholinophenyl)-N,N-dimethylmethanamine (132 mg, 0.441 mmol) to give a mixture of the boronate and boronic acid which was used in the next step without further purification. LCMS [M+H]+ 347.30 Boronate, 265.36 Boronic acid.

### Step 2: 6-(3-amino-6-(3-((dimethylamino)methyl)-4-morpholinophenyl)-5-fluoropyrazin-2-yl)-4-fluoroisoquinolin-1(2H)-one

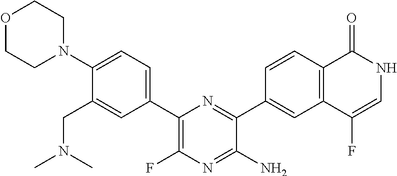 [SMILES: CN(C)Cc1cc(-c2nc(-c3ccc4c(=O)[nH]cc(F)c4c3)c(N)nc2F)ccc1N1CCOCC1]

Prepared from 6-(3-amino-6-bromo-5-fluoropyrazin-2-yl)-4-fluoroisoquinolin-1(2H)-one (45 mg, 0.127 mmol) and N,N-dimethyl-1-(2-morpholino-5-(4,4,5,5-tetramethyl-1,3,2-dioxaborolan-2-yl)phenyl)methanamine (53.0 mg, 0.153 mmol) to give the title compound (14.5 mg, 23.1% yield) as a yellow solid. ¹H NMR (500 MHz, DMSO-d₆) δ ppm 11.21 (br. s., 1H), 8.32 (dd, J=8.25, 1.65 Hz, 1H), 8.06 (d, J=0.98 Hz, 1H), 7.94 (dd, J=8.38, 1.41 Hz, 1H), 7.90 (s, 1H), 7.74 (d, J=8.44 Hz, 1H), 7.45 (d, J=5.62 Hz, 1H), 7.18 (d, J=8.44 Hz, 1H), 6.96 (s, 2H), 3.72-3.79 (m, 4H), 3.46 (s, 2H), 2.93-2.99 (m, 4H), 2.19 (s, 6H); LCMS: [M+H]⁺=493.47.

### Example 90: 6-(3-amino-5-fluoro-6-(4-(4-isopropylpiperazin-1-yl)phenyl)pyrazin-2-yl)-4-chloro-8-fluoroisoquinolin-1(2H)-one (I-90)

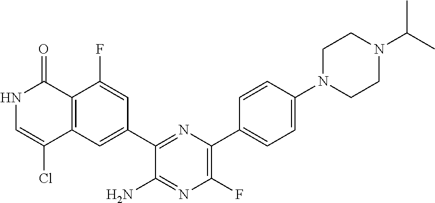 [SMILES: CC(C)N1CCN(c2ccc(-c3nc(-c4cc(F)c5c(=O)[nH]cc(Cl)c5c4)c(N)nc3F)cc2)CC1]

### Step 1: 6-bromo-4-chloro-8-fluoroisoquinolin-1(2H)-one

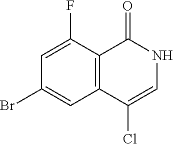 [SMILES: O=c1[nH]cc(Cl)c2cc(Br)cc(F)c12]

To a solution of 6-bromo-8-fluoro-1,2-dihydroisoquinolin-1-one (120 mg, 0.496 mmol) in N,N-dimethylacetamide (2 ml) was added N-chlorosuccinimide (79 mg, 0.595 mmol). The mixture was stirred at 50° C. for 1 hour then cooled to RT and diluted with water. The beige suspension was filtered, washed with water, and dried under vacuum to obtain 6-bromo-4-chloro-8-fluoroisoquinolin-1(2H)-one (111 mg, 0.401 mmol, 81% yield) as a beige solid. The material was used at ˜80% purity (UV 254) in the next step without further purification. ¹H NMR (500 MHz, DMSO-d₆) δ ppm 11.72 (br. s., 1H), 7.74 (s, 1H), 7.71 (d, J=11.00 Hz, 1H), 7.60 (d, J=6.24 Hz, 1H); LCMS: [M+H]⁺=276.15.

### Step 2: (4-chloro-8-fluoro-1-oxo-1,2-dihydroisoquinolin-6-yl)boronic acid

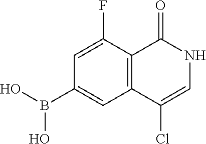 [SMILES: O=c1[nH]cc(Cl)c2cc(B(O)O)cc(F)c12]

Prepared from 6-bromo-4-chloro-8-fluoroisoquinolin-1(2H)-one (110 mg, 0.398 mmol) to give the boronic acid which was used in the next step without further purification. LCMS: [M+H]⁺=242.29.

### Step 3: 6-(2-amino-6-fluoro-5-(4-(4-isopropylpiperazin-1-yl)phenyl)pyridin-3-yl)-4-chloro-8-fluoroisoquinolin-1(2H)-one

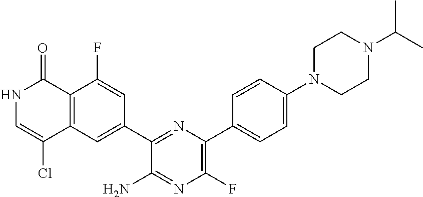 [SMILES: CC(C)N1CCN(c2ccc(-c3nc(-c4cc(F)c5c(=O)[nH]cc(Cl)c5c4)c(N)nc3F)cc2)CC1]

Prepared from (4-chloro-8-fluoro-1-oxo-1,2-dihydroisoquinolin-6-yl)boronic acid (31.8 mg, 0.132 mmol) and 3-bromo-6-fluoro-5-(4-(4-isopropylpiperazin-1-yl)phenyl)pyrazin-2-amine (40 mg, 0.101 mmol) to give the title compound (1.4 mg, 2.70% yield) as a yellow solid. ¹H NMR (500 MHz, DMSO-d₆) δ ppm 11.61 (d, J=5.75 Hz, 1H), 7.97 (s, 1H), 7.75 (d, J=8.07 Hz, 2H), 7.65 (d, J=12.35 Hz, 1H), 7.57 (d, J=6.24 Hz, 1H), 7.03 (d, J=8.19 Hz, 2H), 6.92 (s, 2H), 3.19 (br. s., 4H), 2.68-2.73 (m, 1H), 2.57-2.60 (m, 4H), 1.01 (br. s., 6H); LCMS: [M+H]⁺=511.39.

### Example 91: 7-(3-amino-6-(3-((dimethylamino)methyl)-4-(tetrahydro-2H-pyran-4-yl)phenyl)-5-fluoropyrazin-2-yl)-5-fluoro-2-methylquinazolin-4(3H)-one (I-91)

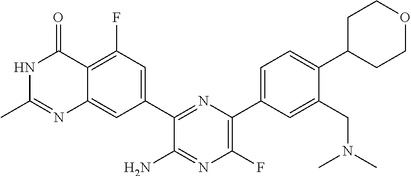 [SMILES: Cc1nc2cc(-c3nc(-c4ccc(C5CCOCC5)c(CN(C)C)c4)c(F)nc3N)cc(F)c2c(=O)[nH]1]

### Step 1: 7-(3-amino-6-bromo-5-fluoropyrazin-2-yl)-5-fluoro-2-methylquinazolin-4(3H)-one

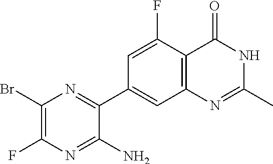 [SMILES: Cc1nc2cc(-c3nc(Br)c(F)nc3N)cc(F)c2c(=O)[nH]1]

7-Bromo-5-fluoro-2-methylquinazolin-4(3H)-one (3.30 g, 12.84 mmol), potassium acetate (3.78 g, 38.5 mmol), 1,1′-nis(diphenylphosphino)ferrocene-palladium(II)dichloride (0.939 g, 1.284 mmol) and bis(pinacolato)diboron (3.59 g, 14.12 mmol) were mixed in 1,4-dioxane (80 ml) under vacuum and stirred under 90° C. oil bath overnight. The reaction mixture was filtered and rinsed with acetonitrile (80 mL). The filtrate was directly carried on to the next step reaction without further purification. Half of the filtrate was mixed with sodium carbonate monohydrate (1.592 g, 12.84 mmol), bis(triphenylphosphine)palladium(II) dichloride (0.541 g, 0.770 mmol) and 5-bromo-6-fluoro-3-iodopyrazin-2-amine (2.245 g, 7.06 mmol) in water (20 ml) under vacuum. The reaction mixture was stirred under 90° C. oil bath overnight. The reaction mixture was filtered, concentrated with silica gel and purified by column chromatography, eluted with 50-100% EtOAc in hexanes The product was triturated with ether to get 7-(3-amino-6-bromo-5-fluoropyrazin-2-yl)-5-fluoro-2-methylquinazolin-4(3H)-one (791 mg, 32.6% yield) as a brown solid. LCMS: [M+H]⁺=368.17.

### Step 2: 7-(3-amino-6-(3-((dimethylamino)methyl)-4-(tetrahydro-2H-pyran-4-yl)phenyl)-5-fluoropyrazin-2-yl)-5-fluoro-2-methylquinazolin-4(3H)-one

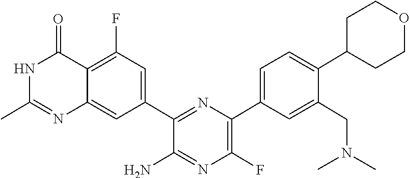 [SMILES: Cc1nc2cc(-c3nc(-c4ccc(C5CCOCC5)c(CN(C)C)c4)c(F)nc3N)cc(F)c2c(=O)[nH]1]

Prepared from 7-(3-amino-6-bromo-5-fluoropyrazin-2-yl)-5-fluoro-2-methylquinazolin-4(3H)-one (40 mg, 0.109 mmol) reacted with N,N-dimethyl-1-(2-(tetrahydro-2H-pyran-4-yl)-5-(4,4,5,5-tetramethyl-1,3,2-dioxaborolan-2 yl)phenyl)methanamine (37.5 mg, 0.109 mmol) to give the title compound (26.7 mg, 47.5% yield). ¹H NMR (500 MHz, METHANOL-d4) δ=7.92-7.80 (m, 3H), 7.59 (dd, J=1.2, 11.6 Hz, 1H), 7.43 (d, J=8.2 Hz, 1H), 4.06 (dd, J=3.7, 11.2 Hz, 2H), 3.72-3.55 (m, 4H), 3.30-3.24 (m, 1H), 2.46 (s, 3H), 2.33 (s, 6H), 1.93-1.81 (m, 2H), 1.73 (br d, J=11.6 Hz, 2H) LCMS: [M+H]⁺=507.51.

### Example 92: 7-(3-amino-6-(3-((dimethylamino)methyl)-4-(tetrahydro-2H-pyran-4-yl)phenyl)-5-fluoropyrazin-2-yl)-2-methylquinazolin-4(3H)-one (I-92)

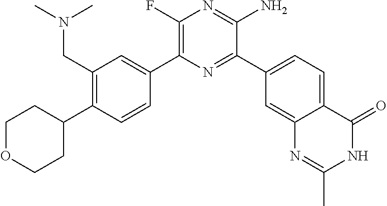 [SMILES: Cc1nc2cc(-c3nc(-c4ccc(C5CCOCC5)c(CN(C)C)c4)c(F)nc3N)ccc2c(=O)[nH]1]

Prepared from 7-(3-amino-6-bromo-5-fluoropyrazin-2-yl)-2-methylquinazolin-4(3H)-one (40 mg, 0.114 mmol) reacted with N,N-dimethyl-1-(2-(tetrahydro-2H-pyran-4-yl)-5-(4,4,5,5-tetramethyl-1,3,2-dioxaborolan-2-yl)phenyl)methanamine (39.4 mg, 0.114 mmol) to give the title compound (32.5 mg, 57.1% yield). ¹H NMR (500 MHz, METHANOL-d₄) δ=8.32 (d, J=8.3 Hz, 1H), 8.05 (s, 1H), 7.96-7.86 (m, 3H), 7.44 (d, J=8.1 Hz, 1H), 4.10-4.05 (m, 2H), 3.70-3.58 (m, 4H), 3.31-3.25 (m, 1H), 2.50 (s, 3H), 2.39-2.30 (m, 6H), 1.95-1.83 (m, 2H), 1.74 (br d, J=11.7 Hz, 2H); LCMS: [M+H]⁺=489.46.

### Example 93: 6-(3-amino-5-fluoro-6-(4-((3R,5S)-3,4,5-trimethylpiperazin-1-yl)phenyl)pyrazin-2-yl)-4-fluoroisoquinolin-1(2H)-one (I-93)

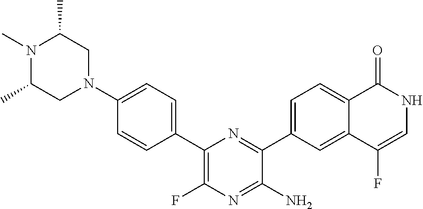 [SMILES: C[C@@H]1CN(c2ccc(-c3nc(-c4ccc5c(=O)[nH]cc(F)c5c4)c(N)nc3F)cc2)C[C@H](C)N1C]

Prepared from 6-(3-amino-6-bromo-5-fluoropyrazin-2-yl)-4-fluoroisoquinolin-1(2H)-one (60 mg, 0.170 mmol) and (2S,6R)-1,2,6-trimethyl-4-(4-(4,4,5,5-tetramethyl-1,3,2-dioxaborolan-2-yl)phenyl)piperazine (112 mg, 0.340 mmol) to give the title compound as a yellow solid, (41 mg, 50.1% yield). ¹H NMR (500 MHz, DMSO-d₆) δ 11.20 (br s, 1H), 8.32 (dd, J=8.3, 1.5 Hz, 1H), 8.05 (s, 1H), 7.95 (dd, J=8.3, 1.1 Hz, 1H), 7.74 (d, J=8.2 Hz, 2H), 7.44 (br d, J=3.4 Hz, 1H), 7.02 (d, J=8.9 Hz, 2H), 6.84 (s, 2H), 3.64 (br d, J=11.2 Hz, 2H), 2.44 (t, J=11.4 Hz, 2H), 2.27-2.21 (m, 2H), 2.19 (s, 3H), 1.08 (d, J=6.1 Hz, 6H); LCMS: [M+H]⁺=477.07.

### Example 94: 6-(3-amino-5-fluoro-6-(4-((3R,5S)-3,4,5-trimethylpiperazin-1-yl)phenyl)pyrazin-2-yl)-4-methylisoquinolin-1(2H)-one (I-94)

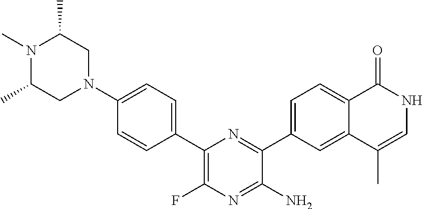 [SMILES: Cc1c[nH]c(=O)c2ccc(-c3nc(-c4ccc(N5C[C@@H](C)N(C)[C@@H](C)C5)cc4)c(F)nc3N)cc12]

### Step 1: 2-(4,4,5,5-tetramethyl-1,3,2-dioxaborolan-2-yl)-6,7-dihydrothieno[3,2-c]pyridin-4(5H)-one

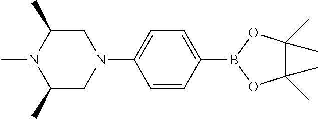

Prepared from (2S,6R)-4-(4-bromophenyl)-1,2,6-trimethylpiperazine (600 mg, 2.119 mmol) to give the product (700 mg, 100% yield). LCMS: [M+H]⁺=332.10.

### Step 2. 6-(3-amino-5-fluoro-6-(4-((3S,5R)-3,4,5-trimethylpiperazin-1-yl)phenyl)pyrazin-2-yl)-4-methylisoquinolin-1(2H)-one

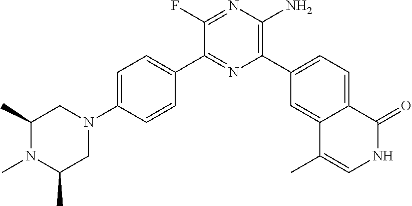 [SMILES: Cc1c[nH]c(=O)c2ccc(-c3nc(-c4ccc(N5C[C@@H](C)N(C)[C@@H](C)C5)cc4)c(F)nc3N)cc12]

Prepared from 6-(3-amino-6-bromo-5-fluoropyrazin-2-yl)-4-methylisoquinolin-1(2H)-one (60 mg, 0.172 mmol) and (2S,6R)-1,2,6-trimethyl-4-(4-(4,4,5,5-tetramethyl-1,3,2-dioxaborolan-2-yl)phenyl)piperazine (114 mg, 0.344 mmol) to give the title compound as a yellow solid (27 mg, 32.6% yield). ¹H NMR (500 MHz, DMSO-d₆) δ 11.14 (br d, J=5.4 Hz, 1H), 8.33 (d, J=8.2 Hz, 1H), 7.98 (s, 1H), 7.84 (d, J=8.2 Hz, 1H), 7.75 (br d, J=8.2 Hz, 2H), 7.06 (br d, J=5.4 Hz, 1H), 7.02 (d, J=8.9 Hz, 2H), 6.81 (s, 2H), 3.64 (br d, J=11.4 Hz, 2H), 2.43 (t, J=11.4 Hz, 2H), 2.26 (s, 3H), 2.25-2.21 (m, 2H), 2.19 (s, 3H), 1.08 (d, J=6.1 Hz, 6H); LCMS: [M+1]⁺=473.38.

### Example 95: 7-(3-amino-5-fluoro-6-(4-((3R,5S)-3,4,5-trimethylpiperazin-1-yl)phenyl)pyrazin-2-yl)-2-methylquinazolin-4(3H)-one (I-95)

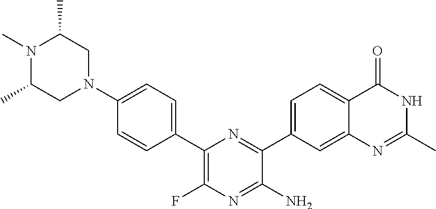

Prepared from 7-(3-amino-6-bromo-5-fluoropyrazin-2-yl)-2-methylquinazolin-4(3H)-one (60 mg, 0.171 mmol) and (2S,6R)-1,2,6-trimethyl-4-(4-(4,4,5,5-tetramethyl-1,3,2-dioxaborolan-2-yl)phenyl)piperazine (113 mg, 0.343 mmol) to give the title compound as a yellow solid (49 mg, 59.8% yield). ¹H NMR (500 MHz, DMSO-d₆) δ 12.25 (br s, 1H), 8.17 (d, J=8.2 Hz, 1H), 7.90 (d, J=1.0 Hz, 1H), 7.79 (dd, J=8.3, 1.4 Hz, 1H), 7.75 (d, J=8.1 Hz, 2H), 7.01 (d, J=8.9 Hz, 2H), 6.79 (s, 2H), 3.64 (br d, J=11.1 Hz, 2H), 2.43 (t, J=11.4 Hz, 2H), 2.38 (s, 3H), 2.27-2.21 (m, 2H), 2.19 (s, 3H), 1.08 (d, J=6.1 Hz, 6H); LCMS: [M+1]⁺=474.51.

### Example 96: 6-(3-amino-6-(3-((dimethylamino)methyl)-4-morpholinophenyl)-5-fluoropyrazin-2-yl)-4-methylisoquinolin-1(2H)-one (I-96)

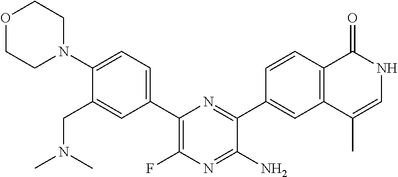 [SMILES: Cc1c[nH]c(=O)c2ccc(-c3nc(-c4ccc(N5CCOCC5)c(CN(C)C)c4)c(F)nc3N)cc12]

Prepared from 7-(3-amino-6-bromo-5-fluoropyrazin-2-yl)-2-methylquinazolin-4(3H)-one (60 mg, 0.171 mmol) and (2S,6R)-1,2,6-trimethyl-4-(4-(4,4,5,5-tetramethyl-1,3,2-dioxaborolan-2-yl)phenyl)piperazine (113 mg, 0.343 mmol) to give the title compound as a yellow solid (49 mg, 59.8% yield). ¹H NMR (500 MHz, DMSO-d₆) δ 12.25 (br s, 1H), 8.17 (d, J=8.2 Hz, 1H), 7.90 (d, J=1.0 Hz, 1H), 7.79 (dd, J=8.3, 1.4 Hz, 1H), 7.75 (d, J=8.1 Hz, 2H), 7.01 (d, J=8.9 Hz, 2H), 6.79 (s, 2H), 3.64 (br d, J=11.1 Hz, 2H), 2.43 (t, J=11.4 Hz, 2H), 2.38 (s, 3H), 2.27-2.21 (m, 2H), 2.19 (s, 3H), 1.08 (d, J=6.1 Hz, 6H); LCMS: [M+1]⁺=474.51.

### Example 97: 6-(3-amino-6-(3-((dimethylamino)methyl)-4-(tetrahydro-2H-pyran-4-yl)phenyl)-5-fluoropyrazin-2-yl)-4-fluoro-3-methylisoquinolin-1(2H)-one (I-97)

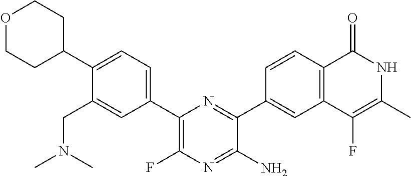 [SMILES: Cc1[nH]c(=O)c2ccc(-c3nc(-c4ccc(C5CCOCC5)c(CN(C)C)c4)c(F)nc3N)cc2c1F]

### Step 1: 6-bromo-4-fluoro-3-methylisoquinolin-1(2H)-one

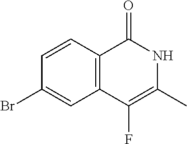

A vial was charged with 6-bromo-3-methyl-2H-isoquinolin-1-one (1000 mg, 4.20 mmol) and Selectfluor™ fluorinating reagent >95% in F+ active (1711 mg, 4.83 mmol). Methanol (15 ml) and acetonitrile (15 ml) were added, and the reaction was stirred at room temperature for 5 days. The white suspension was filtered, washing with water, and dried under vacuum to afford 6-bromo-4-fluoro-3-methylisoquinolin-1(2H)-one (903 mg, 3.53 mmol, 84% yield) as a white solid. ¹H NMR (500 MHz, DMSO-d₆) δ ppm 11.38 (br. s., 1H), 8.07 (dd, J=8.50, 1.65 Hz, 1H), 7.83 (s, 1H), 7.69 (dd, J=8.44, 1.59 Hz, 1H), 2.22 (d, J=3.18 Hz, 3H); LCMS [M+H]⁺=256.15.

### Step 2: 4-fluoro-3-methyl-6-(4,4,5,5-tetramethyl-1,3,2-dioxaborolan-2-yl)isoquinolin-1(2H)-one

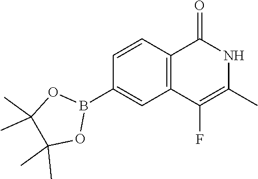

Prepared from 6-bromo-4-fluoro-3-methylisoquinolin-1(2H)-one (850 mg, 3.32 mmol) to give a mixture of boronate and boronic acid which was used in the next step without further purification. LCMS[M+H]+ 222.21 Boronic acid, 304.20 Boronate.

### Step 3: 6-(3-amino-6-bromo-5-fluoropyrazin-2-yl)-4-fluoro-3-methylisoquinolin-1(2H)-one

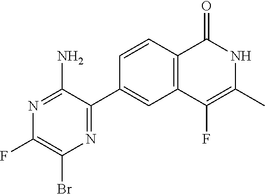 [SMILES: Cc1[nH]c(=O)c2ccc(-c3nc(Br)c(F)nc3N)cc2c1F]

Prepared from 5-bromo-6-fluoro-3-iodopyrazin-2-amine (430 mg, 1.353 mmol) and 4-fluoro-3-methyl-6-(4,4,5,5-tetramethyl-1,3,2-dioxaborolan-2-yl)isoquinolin-1(2H)-one (492 mg, 1.623 mmol) to give the product (317 mg, 63.8% yield) as a beige solid. LCMS: [M+H]⁺=367.23.

### Step 4: 6-(3-amino-6-(3-((dimethylamino)methyl)-4-(tetrahydro-2H-pyran-4-yl)phenyl)-5-fluoropyrazin-2-yl)-4-fluoro-3-methylisoquinolin-1(2H)-one

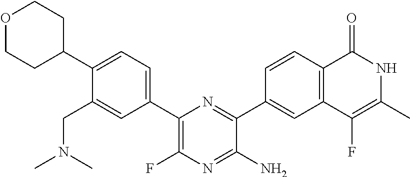 [SMILES: Cc1[nH]c(=O)c2ccc(-c3nc(-c4ccc(C5CCOCC5)c(CN(C)C)c4)c(F)nc3N)cc2c1F]

Prepared from 6-(3-amino-6-bromo-5-fluoropyrazin-2-yl)-4-fluoro-3-methylisoquinolin-1(2H)-one (40 mg, 0.109 mmol) and N,N-dimethyl-1-(2-(tetrahydro-2H-pyran-4-yl)-5-(4,4,5,5-tetramethyl-1,3,2-dioxaborolan-2-yl)phenyl)methanamine (45.1 mg, 0.131 mmol) to give the title compound (36.6 mg, 66.4% yield) as a yellow solid. ¹H NMR (500 MHz, DMSO-d₆) δ ppm 11.31 (br. s., 1H), 8.27 (dd, J=8.25, 1.65 Hz, 1H), 7.98 (d, J=0.98 Hz, 1H), 7.86 (dd, J=8.31, 1.34 Hz, 1H), 7.75 (d, J=8.31 Hz, 1H), 7.73 (s, 1H), 7.40 (d, J=8.19 Hz, 1H), 6.97 (s, 2H), 3.96 (dd, J=10.64, 3.67 Hz, 2H), 3.42-3.47 (m, 4H), 3.17-3.25 (m, 1H), 2.25 (d, J=2.93 Hz, 3H), 2.17 (s, 6H), 1.72 (qd, J=12.31, 4.16 Hz, 2H), 1.59-1.65 (m, 2H); LCMS [M+H]⁺=506.44.

### Example 98: 6-(3-amino-6-(3-((dimethylamino)methyl)-4-(tetrahydro-2H-pyran-4-yl)phenyl)-5-fluoropyrazin-2-yl)-4-methylisoquinolin-1(2H)-one (I-98)

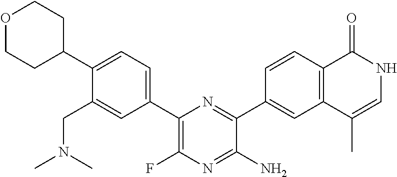 [SMILES: Cc1c[nH]c(=O)c2ccc(-c3nc(-c4ccc(C5CCOCC5)c(CN(C)C)c4)c(F)nc3N)cc12]

Prepared from 6-(3-amino-6-bromo-5-fluoropyrazin-2-yl)-4-methylisoquinolin-1(2H)-one (40 mg, 0.115 mmol) and N,N-dimethyl-1-(2-(tetrahydro-2H-pyran-4-yl)-5-(4,4,5,5-tetramethyl-1,3,2-dioxaborolan-2-yl)phenyl)methanamine (47.5 mg, 0.137 mmol) to give the title compound (15.3 mg, 27.4% yield) as a beige solid. ¹H NMR (500 MHz, DMSO-d₆) δ ppm 11.14 (d, J=4.89 Hz, 1H), 8.33 (d, J=8.19 Hz, 1H), 7.99 (s, 1H), 7.84 (d, J=8.19 Hz, 1H), 7.71-7.80 (m, 2H), 7.40 (d, J=8.56 Hz, 1H), 7.07 (d, J=4.77 Hz, 1H), 6.95 (br. s., 2H), 3.96 (d, J=8.07 Hz, 2H), 3.41-3.51 (m, 4H), 3.21 (t, J=11.86 Hz, 1H), 2.26 (s, 3H), 2.17 (s, 6H), 1.67-1.80 (m, 2H), 1.58-1.66 (m, 2H); LCMS: [M+H]⁺=488.52.

### Example 99: 6-(3-amino-6-(4-((1S,5R)-3-cyclobutyl-3-azabicyclo[3.1.0]hexan-1-yl)phenyl)-5-fluoropyrazin-2-yl)-4-fluoroisoquinolin-1(2H)-one (I-99)

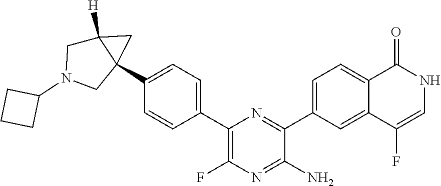 [SMILES: Nc1nc(F)c(-c2ccc([C@]34C[C@H]3CN(C3CCC3)C4)cc2)nc1-c1ccc2c(=O)[nH]cc(F)c2c1]

Prepared from 6-(3-amino-6-bromo-5-fluoropyrazin-2-yl)-4-fluoroisoquinolin-1(2H)-one (40 mg, 0.113 mmol) reacted with (1 S,5R)-3-cyclobutyl-1-(4-(4,4,5,5-tetramethyl-1,3,2-dioxaborolan-2-yl)phenyl)-3-azabicyclo[3.1.0]hexane (38.4 mg, 0.113 mmol, prepared by standard methods analogous to those described in earlier procedures) to give the title compound (12.4 mg 22.1% yield). ¹H NMR (500 MHz, METHANOL-d₄) δ=8.47 (dd, J=1.7, 8.4 Hz, 1H), 8.25 (d, J=1.2 Hz, 1H), 8.07 (dd, J=1.6, 8.4 Hz, 1H), 7.90 (d, J=7.3 Hz, 2H), 7.30 (d, J=5.5 Hz, 1H), 7.27 (d, J=8.4 Hz, 2H), 3.41-3.32 (m, 1H), 3.20-3.11 (m, 1H), 3.08 (d, J=9.2 Hz, 1H), 2.70 (d, J=8.9 Hz, 1H), 2.61 (dd, J=3.6, 9.1 Hz, 1H), 2.12-1.96 (m, 4H), 1.87 (td, J=4.0, 8.1 Hz, 1H), 1.81-1.71 (m, 2H), 1.45 (t, J=4.4 Hz, 1H), 0.90 (dd, J=4.5, 8.1 Hz, 1H); LCMS: [M+H]⁺=486.57;

### Example 100: 6-(3-amino-6-(3-((dimethylamino)methyl)-4-(tetrahydro-2H-pyran-4-yl)phenyl)-5-fluoropyrazin-2-yl)-8-fluoro-3-methylisoquinolin-1(2H)-one (I-100)

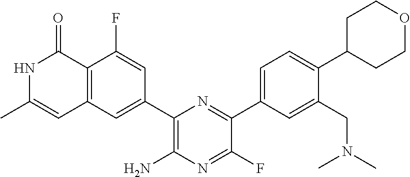 [SMILES: Cc1cc2cc(-c3nc(-c4ccc(C5CCOCC5)c(CN(C)C)c4)c(F)nc3N)cc(F)c2c(=O)[nH]1]

### Step 1: 6-(3-amino-6-bromo-5-fluoropyrazin-2-yl)-8-fluoro-3-methylisoquinolin-1(2H)-one

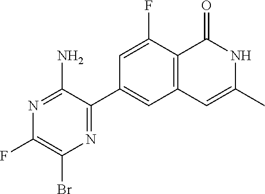 [SMILES: Cc1cc2cc(-c3nc(Br)c(F)nc3N)cc(F)c2c(=O)[nH]1]

Prepared from 5-bromo-6-fluoro-3-iodopyrazin-2-amine (450 mg, 1.416 mmol) and 8-fluoro-3-methyl-6-(4,4,5,5-tetramethyl-1,3,2-dioxaborolan-2-yl)isoquinolin-1(2H)-one (558 mg, 1.840 mmol) to give the product (119 mg, 22.9% yield) as a beige solid. LCMS: [M+H]⁺=367.30.

### Step 2: 6-(3-amino-6-(3-((dimethylamino)methyl)-4-(tetrahydro-2H-pyran-4-yl)phenyl)-5-fluoropyrazin-2-yl)-8-fluoro-3-methylisoquinolin-1(2H)-one

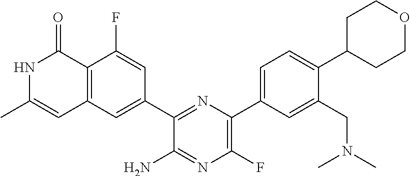 [SMILES: Cc1cc2cc(-c3nc(-c4ccc(C5CCOCC5)c(CN(C)C)c4)c(F)nc3N)cc(F)c2c(=O)[nH]1]

Prepared from 6-(3-amino-6-bromo-5-fluoropyrazin-2-yl)-8-fluoro-3-methylisoquinolin-1(2H)-one (40 mg, 0.109 mmol) and N,N-dimethyl-1-(2-(tetrahydro-2H-pyran-4-yl)-5-(4,4,5,5-tetramethyl-1,3,2-dioxaborolan-2-yl)phenyl)methanamine (45.1 mg, 0.131 mmol) to give the title compound (15.9 mg, 28.9% yield) as a yellow solid. ¹H NMR (500 MHz, DMSO-d₆) δ ppm 11.32 (br. s., 1H), 7.75 (d, J=8.19 Hz, 1H), 7.71 (s, 1H), 7.66 (d, J=1.22 Hz, 1H), 7.35-7.43 (m, 2H), 6.98 (s, 2H), 6.41 (s, 1H), 3.96 (dd, J=10.70, 3.48 Hz, 2H), 3.42-3.48 (m, 4H), 3.21 (ddd, J=11.58, 8.28, 3.61 Hz, 1H), 2.21 (s, 3H), 2.17 (s, 6H), 1.72 (qd, J=12.23, 4.03 Hz, 2H), 1.59-1.65 (m, 2H); LCMS: [M+H]⁺=506.50.

### Example 101: 6-(3-amino-6-(3-((dimethylamino)methyl)-4-morpholinophenyl)-5-fluoropyrazin-2-yl)-4-fluoro-3-methylisoquinolin-1(2H)-one (I-101)

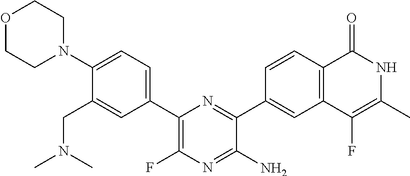 [SMILES: Cc1[nH]c(=O)c2ccc(-c3nc(-c4ccc(N5CCOCC5)c(CN(C)C)c4)c(F)nc3N)cc2c1F]

Prepared from 6-(3-amino-6-bromo-5-fluoropyrazin-2-yl)-4-fluoro-3-methylisoquinolin-1(2H)-one (35 mg, 0.095 mmol) and N,N-dimethyl-1-(2-morpholino-5-(4,4,5,5-tetramethyl-1,3,2-dioxaborolan-2-yl)phenyl)methanamine (36.3 mg, 0.105 mmol) to give the title compound (3.4 mg, 7.04% yield) as a yellow solid. ¹H NMR (500 MHz, DMSO-d₆) δ ppm 11.25 (br. s., 1H), 8.20 (dd, J=8.25, 1.77 Hz, 1H), 7.92 (d, J=1.10 Hz, 1H), 7.84 (br. s., 1H), 7.79 (dd, J=8.25, 1.53 Hz, 1H), 7.69 (d, J=6.97 Hz, 1H), 7.13 (br. s., 1H), 6.88 (br. s., 2H), 3.67-3.72 (m, 4H), 3.42 (br. s., 2H), 2.88 (br. s., 4H), 2.19 (d, J=2.93 Hz, 3H), 2.16 (m, J=9.30 Hz, 6H); LCMS: [M+H]⁺=507.26.

### Example 102: 6-(3-amino-5-fluoro-6-(4-(4-isopropylpiperazin-1-yl)phenyl)pyrazin-2-yl)-4-chloro-8-fluoro-3-methylisoquinolin-1(2H)-one (I-102)

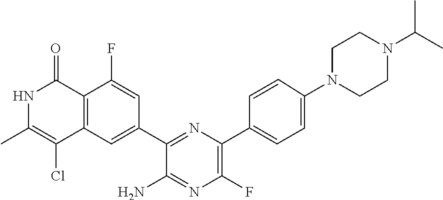 [SMILES: Cc1[nH]c(=O)c2c(F)cc(-c3nc(-c4ccc(N5CCN(C(C)C)CC5)cc4)c(F)nc3N)cc2c1Cl]

### Step 1: 6-bromo-4-chloro-8-fluoro-3-methylisoquinolin-1(2H)-one

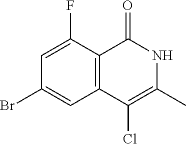

To a solution of 6-bromo-8-fluoro-3-methylisoquinolin-1(2H)-one (500 mg, 1.953 mmol) in N,N-dimethylacetamide (14 ml) was added N-chlorosuccinimide (313 mg, 2.343 mmol). The mixture was stirred at 50° C. for 2 hours. The reaction was cooled to room temperature and diluted with water. The suspension was filtered, washed with water, triturated from methanol and dried under vacuum to the product (188 mg, 33.1% yield) as a white solid. LCMS: [M+H]⁺=290.19.

### Step 2: (4-chloro-8-fluoro-3-methyl-1-oxo-1,2-dihydroisoquinolin-6-yl)boronic acid

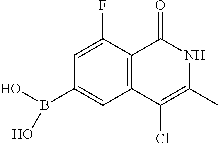 [SMILES: Cc1[nH]c(=O)c2c(F)cc(B(O)O)cc2c1Cl]

Prepared from 6-bromo-4-chloro-8-fluoro-3-methylisoquinolin-1(2H)-one (185 mg, 0.637 mmol) to give the boronic acid which was used in the next step without further purification. LCMS[M+H]⁺256.13.

### Step 3: 6-(3-amino-6-bromo-5-fluoropyrazin-2-yl)-4-chloro-8-fluoro-3-methylisoquinolin-1(2H)-one

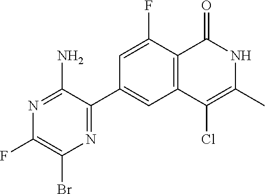 [SMILES: Cc1[nH]c(=O)c2c(F)cc(-c3nc(Br)c(F)nc3N)cc2c1Cl]

Prepared from 5-bromo-6-fluoro-3-iodopyrazin-2-amine (168 mg, 0.528 mmol) and (4-chloro-8-fluoro-3-methyl-1-oxo-1,2-dihydroisoquinolin-6-yl)boronic acid (162 mg, 0.634 mmol) to give the product; LCMS: [M+H]⁺=401.21.

### Step 4: 6-(3-amino-5-fluoro-6-(4-(4-isopropylpiperazin-1-yl)phenyl)pyrazin-2-yl)-4-chloro-8-fluoro-3-methylisoquinolin-1(2H)-one

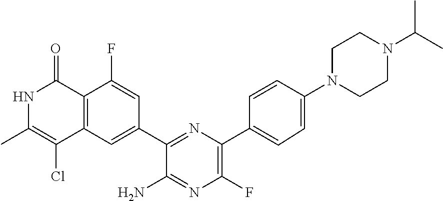 [SMILES: Cc1[nH]c(=O)c2c(F)cc(-c3nc(-c4ccc(N5CCN(C(C)C)CC5)cc4)c(F)nc3N)cc2c1Cl]

Prepared from 6-(3-amino-6-bromo-5-fluoropyrazin-2-yl)-4-chloro-8-fluoro-3-methylisoquinolin-1(2H)-one (35 mg, 0.087 mmol) and 4-(4-isopropylpiperazinyl)phenylboronic acid, pinacol ester (34.5 mg, 0.105 mmol) to give the title compound (15.6 mg, 34.1% yield) as a yellow solid. ¹H NMR (500 MHz, DMSO-d₆) δ ppm 11.70 (br. s., 1H), 7.98 (d, J=0.98 Hz, 1H), 7.74 (d, J=7.95 Hz, 2H), 7.57 (d, J=12.10 Hz, 1H), 7.02 (d, J=8.93 Hz, 2H), 6.89 (s, 2H), 3.17-3.22 (m, 4H), 2.64-2.72 (m, 1H), 2.56-2.59 (m, 4H), 2.37 (s, 3H), 1.00 (d, J=6.60 Hz, 6H); LCMS: [M+H]⁺=525.49.

### Example 103: 6′-(3-amino-5-fluoro-6-(4-(4-isopropylpiperazin-1-yl)phenyl)pyrazin-2-yl)-2′,3′-dihydro-1′H-spiro[cyclopropane-1,4′-isoquinolin]-1′-one (I-103)

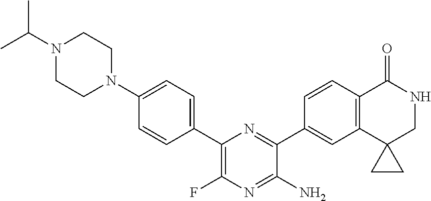 [SMILES: CC(C)N1CCN(c2ccc(-c3nc(-c4ccc5c(c4)C4(CC4)CNC5=O)c(N)nc3F)cc2)CC1]

### Step 1: 6-bromo-1-methylene-2,3-dihydro-1H-indene

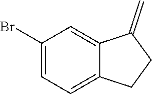

Methytriphenyl phosphonium bromide (101.12 g, 284.27 mmol) was dissolved in tetrahydrofuran (500 mL) followed by addition of potassium tert-butoxide (31.9 g, 284.27 mmol). Upon completion of the addition, the mixture was stirred at RT for 30 minutes. 6-Bromo-indan-1-one (20 g, 94.71 mmol) was dissolved in tetrahydrofuran (100 mL) stirring under nitrogen, treated with the ylide and the resulting mixture was stirred at RT for 1 h. The mixture was diluted with water (300 mL) and extracted with DCM (3×500 mL). The combined organic layer was back-washed with brine solution (2×500 mL), dried over Na₂SO₄ and concentrated under reduced pressure. The resulting crude product was purified by column chromatography over silica gel (100-200 mesh) and the product was eluted in 0-10% EtOAc in hexanes to afford the product (19 g, 95.9% yield) as a yellow oil. ¹H NMR (400 MHz, Chloroform-d) δ 7.65 (s, 1H), 7.36 (d, J=7.3 Hz, 1H), 7.17 (d, J=8.1 Hz, 1H), 5.38 (s, 1H), 5.11 (s, 1H), 3.01-2.93 (m, 2H), 2.89-2.81 (m, 2H).

### Step 2: 6′-bromo-2′,3′-dihydrospiro[cyclopropane-1,1′-indene

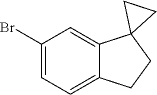 [SMILES: Brc1ccc2c(c1)C1(CC2)CC1]

The 1 M solution of diethyl zinc in hexanes (363.6 mL, 363.3 mmol) was added to a reaction vessel containing DCM (318 mL) and the mixture was cooled to 0° C. TFA (41.4 g, 363.3 mmol) was added and the reaction was further stirred at 0° C. for 15 minutes. To the cooled solution, diiodomethane (97 g, 363.3 mmol) was added and mixture was stirred for an additional 15 minutes at 0° C. After 15 min, a solution of 6-bromo-1-methylene-2,3-dihydro-1H-indene (19 g, 90.87 mmol) in DCM (100 mL) was added and reaction mass was gradually warm to RT and stirred for 16 h. The reaction was quenched with a sat. aq. NH₄Cl (500 mL) and extracted with DCM (3×300 mL). The combined organic layers were back-washed with brine solution (2×200 mL), dried over Na₂SO₄ and concentrated under reduced pressure. The resulting crude product was purified by column chromatography over silica gel (100-200 mesh) eluting with hexanes to afford the title compound (16 g, 78.9% yield) as a clear oil. ¹H NMR (400 MHz, Chloroform-d) δ 7.22 (dd, J=7.9, 1.9 Hz, 1H), 7.05 (d, J=7.8 Hz, 1H), 6.78 (d, J=1.8 Hz, 1H), 2.99 (t, J=7.5 Hz, 2H), 2.06-2.20 (t, 2H), 0.83-1.03 (m, 5H).

### Step 3: Synthesis of 6′-bromospiro[cyclopropane-1,1′-inden]-3′(2′H)-one

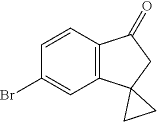 [SMILES: O=C1CC2(CC2)c2cc(Br)ccc21]

To a solution of 6′-bromo-2′,3′-dihydrospiro[cyclopropane-1,1′-indene (16 g, 71.71 mmol) in acetone (224 mL) and 1.5 M aq. MgSO₄ (80 mL) was added KMnO₄ (12.4 g, 78.5 mmol) and the reaction was stirred overnight at room temperature. After completion, the reaction was filtered over celite and concentrated. The crude was diluted with EtOAc (200 mL), washed with brine solution (2×100 mL), dried over Na₂SO₄ and concentrated under reduced pressure. The resulting crude product was purified by column chromatography over silica gel (100-200 mesh) eluting with 0-10% EtOAc in hexanes to give the product (10 g, 58.81%) as a white solid. LCMS: [M+2H]⁺=238.1.

### Step 4: (Z)-6′-bromospiro[cyclopropane-1,1′-inden]-3′(2′H)-one oxime

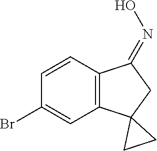 [SMILES: O/N=C1/CC2(CC2)c2cc(Br)ccc21]

A solution of 6′-bromospiro[cyclopropane-1,1′-inden]-3′(2′H)-one (10 g, 42.18 mmol), hydroxylamine HCl (5.5 g, 79.79 mmol) and NaOAc (20.3 g, 247.7 mmol) in MeOH (800 mL) was stirred at RT for 16 h. The reaction mixture was concentrated, slurried with water (40 mL), sonicated and filtered. The precipitate was dried under high vacuum to give the product (8 g, 75.2% yield) as a white solid. LCMS: [M+2H]⁺=253.1.

### Step 5: (Z)-6′-bromospiro[cyclopropane-1,1′-inden]-3′(2′H)-one O-methylsulfonyl oxime

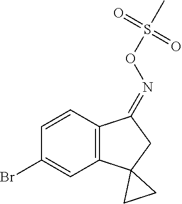 [SMILES: CS(=O)(=O)O/N=C1/CC2(CC2)c2cc(Br)ccc21]

A cooled solution of (E)-6′-bromospiro[cyclopropane-1,1′-inden]-3′(2′H)-one oxime (8 g, 31.73 mmol) in THF (240 mL) at 0° C., was added triethylamine (6.4 g, 63.46 mmol) followed by slow addition of MsCl (5.4 g, 47.6 mmol). The reaction was stirred at 0° C. for 30 min, and after 30 min the reaction was concentrated and the residue was triturated with methanol (4-5 mL) and dried to afford the product (5.5 g, 52.5% yield) as white solid. LCMS: [M+2H]⁺=331.2.

### Step 6: 6′-bromo-2′,3′-dihydro-1′H-spiro[cyclopropane-1,4′-isoquinolin]-1′-onebenzoate

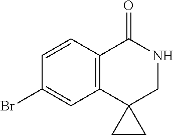 [SMILES: O=C1NCC2(CC2)c2cc(Br)ccc21]

To a cooled solution of (E)-6′-bromospiro[cyclopropane-1,1′-inden]-3′(2′H)-one-O-methylsulfonyl oxime (5.5 g, 16.66 mmol) in DCM (330 mL) at 0° C. was added BF₃MeOH (5.2 g, 36.66 mmol). TiCl₄(4.4 g, 23.33 mmol) was then added slowly and the reaction was stirred at 0° C. for 4 h. The reaction was quenched with water (50 mL) and extracted with DCM (2×100 mL). The combined organics were dried over Na₂SO₄ and concentrated under reduced pressure. The resulting crude product was purified by column chromatography over silica gel (100-200 mesh) eluting with 50-100% EtOAc in hexanes to afford the product (1 g, 23.8%) as white solid. LCMS: [M+2H]⁺=253.1.

### Step 7: 6′-(4,4,5,5-tetramethyl-1,3,2-dioxaborolan-2-yl)-2′,3′-dihydro-1′H-spiro[cyclopropane-1,4′-isoquinolin]-1′-one

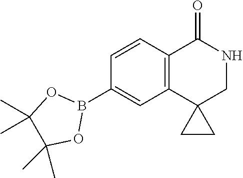 [SMILES: CC1(C)OB(c2ccc3c(c2)C2(CC2)CNC3=O)OC1(C)C]

Prepared from 6′-bromo-2′,3′-dihydro-1′H-spiro[cyclopropane-1,4′-isoquinolin]-1′-one (0.95 g, 3.77 mmol) to give a mixture of the boronate and boronic acid which was used in the next step without further purification. LCMS[M+H]⁺218.21 Boronic acid, 300.28 Boronate.

### Step 8: 6′-(3-amino-6-bromo-5-fluoropyrazin-2-yl)-2′,3′-dihydro-1′H-spiro[cyclopropane-1,4′-isoquinolin]-1′-one

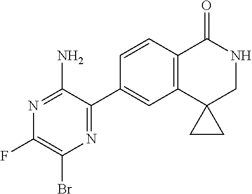 [SMILES: Nc1nc(F)c(Br)nc1-c1ccc2c(c1)C1(CC1)CNC2=O]

mmol) and 6′-(4,4,5,5-tetramethyl-1,3,2-dioxaborolan-2-yl)-2′,3′-dihydro-1′H-spiro[cyclopropane-1,4′-isoquinolin]-1′-one (503 mg, 1.680 mmol) to give the product (397 mg, 78% yield) as a beige solid. LCMS: [M+H]⁺=363.33.

### Step 9: 6′-(3-amino-5-fluoro-6-(4-(4-isopropylpiperazin-1-yl)phenyl)pyrazin-2-yl)-2′,3′-dihydro-1′H-spiro[cyclopropane-1,4′-isoquinolin]-1′-one

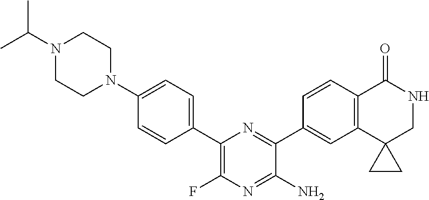 [SMILES: CC(C)N1CCN(c2ccc(-c3nc(-c4ccc5c(c4)C4(CC4)CNC5=O)c(N)nc3F)cc2)CC1]

Prepared from 6′-(3-amino-6-bromo-5-fluoropyrazin-2-yl)-2′,3′-dihydro-1′H-spiro[cyclopropane-1,4′-isoquinolin]-1′-one (35 mg, 0.096 mmol) and 4-(4-isopropylpiperazinyl)phenylboronic acid, pinacol ester (38.2 mg, 0.116 mmol) to give the title compound (15.1 mg, 32.2% yield) as a yellow solid. ¹H NMR (500 MHz, DMSO-d₆) δ ppm 8.07 (br. s., 1H), 7.99 (d, J=8.07 Hz, 1H), 7.72 (d, J=7.82 Hz, 2H), 7.63 (dd, J=8.01, 1.53 Hz, 1H), 7.27 (d, J=1.35 Hz, 1H), 7.00 (d, J=9.05 Hz, 2H), 6.66 (s, 2H), 3.27 (d, J=2.57 Hz, 2H), 3.16-3.20 (m, 4H), 2.64-2.71 (m, 1H), 2.56-2.60 (m, 4H), 1.12-1.16 (m, 2H), 1.00 (d, J=6.48 Hz, 8H); LCMS: [M+H]⁺=487.51.

### Example 104: 6-(3-amino-6-(3-((dimethylamino)methyl)-4-(tetrahydro-2H-pyran-4-yl)phenyl)-5-fluoropyrazin-2-yl)-4-chloro-8-fluoro-3-methylisoquinolin-1(2H)-one (I-104)

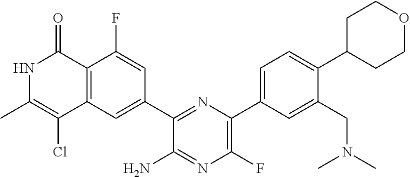 [SMILES: Cc1[nH]c(=O)c2c(F)cc(-c3nc(-c4ccc(C5CCOCC5)c(CN(C)C)c4)c(F)nc3N)cc2c1Cl]

Prepared from 6-(3-amino-6-bromo-5-fluoropyrazin-2-yl)-4-chloro-8-fluoro-3-methylisoquinolin-1(2H)-one (40 mg, 0.100 mmol) and N,N-dimethyl-1-(2-(tetrahydro-2H-pyran-4-yl)-5-(4,4,5,5-tetramethyl-1,3,2-dioxaborolan-2-yl)phenyl)methanamine (41.3 mg, 0.120 mmol) to give the title compound (7.1 mg, 13.2% yield) as a yellow solid. ¹H NMR (500 MHz, DMSO-d₆) δ ppm 11.70 (br. s., 1H), 7.99 (d, J=0.98 Hz, 1H), 7.74 (br. s., 2H), 7.56-7.61 (m, 1H), 7.41 (d, J=8.80 Hz, 1H), 7.05 (s, 2H), 3.96 (dd, J=10.70, 3.61 Hz, 2H), 3.42-3.48 (m, 4H), 3.17-3.25 (m, 1H), 2.37 (s, 3H), 2.17 (s, 6H), 1.72 (qd, J=12.29, 4.10 Hz, 2H), 1.59-1.66 (m, 2H); LCMS: [M+H]⁺=540.41

### Example 105: 6′-(3-amino-6-(3-((dimethylamino)methyl)-4-morpholinophenyl)-5-fluoropyrazin-2-yl)-2′,3′-dihydro-1′H-spiro[cyclopropane-1,4′-isoquinolin]-1′-one (I-105)

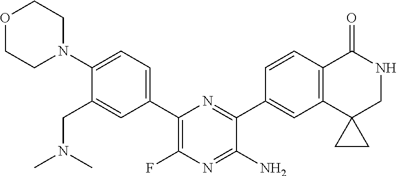 [SMILES: CN(C)Cc1cc(-c2nc(-c3ccc4c(c3)C3(CC3)CNC4=O)c(N)nc2F)ccc1N1CCOCC1]

Prepared from 6′-(3-amino-6-bromo-5-fluoropyrazin-2-yl)-2′,3′-dihydro-1′H-spiro[cyclopropane-1,4′-isoquinolin]-1′-one (40 mg, 0.110 mmol) and N,N-dimethyl-1-(2-morpholino-5-(4,4,5,5-tetramethyl-1,3,2-dioxaborolan-2-yl)phenyl)methanamine (45.8 mg, 0.132 mmol) to give the title compound (16.2 mg, 29.3% yield) as a yellow solid. ¹H NMR (500 MHz, DMSO-d₆) δ ppm 8.08 (br. s., 1H), 7.99 (d, J=8.07 Hz, 1H), 7.90 (s, 1H), 7.72 (d, J=8.56 Hz, 1H), 7.63 (dd, J=8.07, 1.47 Hz, 1H), 7.29 (d, J=1.22 Hz, 1H), 7.16 (d, J=8.44 Hz, 1H), 6.78 (s, 2H), 3.73-3.77 (m, 4H), 3.46 (s, 2H), 3.27 (d, J=2.45 Hz, 2H), 2.93-2.97 (m, 4H), 2.19 (s, 6H), 1.11-1.16 (m, 2H), 0.98-1.03 (m, 2H); LCMS: [M+H]⁺=503.56.

### Example 106: 6-(3-amino-6-(4-(4-(cyclopropylmethyl)piperazin-1-yl)phenyl)-5-fluoropyrazin-2-yl)-4-fluoro-3-methylisoquinolin-1(2H)-one (I-106)

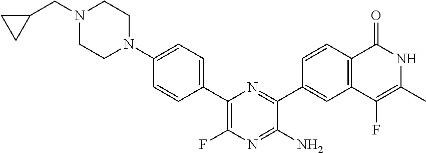 [SMILES: Cc1[nH]c(=O)c2ccc(-c3nc(-c4ccc(N5CCN(CC6CC6)CC5)cc4)c(F)nc3N)cc2c1F]

Prepared from 6-(3-amino-6-bromo-5-fluoropyrazin-2-yl)-4-fluoro-3-methylisoquinolin-1(2H)-one (35 mg, 0.095 mmol) and 1-(cyclopropylmethyl)-4-(4-(4,4,5,5-tetramethyl-1,3,2-dioxaborolan-2-yl)phenyl)piperazine (39.2 mg, 0.114 mmol) to give the title compound (16.7 mg, 34.9% yield) as a yellow solid. ¹H NMR (500 MHz, DMSO-d₆) δ ppm 11.31 (br. s., 1H), 8.26 (dd, J=8.31, 1.71 Hz, 1H), 7.99 (d, J=0.98 Hz, 1H), 7.87 (dd, J=8.38, 1.41 Hz, 1H), 7.75 (d, J=7.95 Hz, 2H), 7.02 (d, J=9.05 Hz, 2H), 6.83 (s, 2H), 3.19-3.24 (m, 4H), 2.56-2.61 (m, 4H), 2.25 (d, J=3.06 Hz, 3H), 2.23 (d, J=6.60 Hz, 2H), 0.82-0.91 (m, 1H), 0.45-0.51 (m, 2H), 0.08-0.13 (m, 2H); LCMS: [M+H]⁺=503.50.

### Example 107: 6-(3-amino-6-(4-(4-(cyclopropylmethyl)piperazin-1-yl)phenyl)-5-fluoropyrazin-2-yl)-4-chloro-8-fluoro-3-methylisoquinolin-1(2H)-one (I-107)

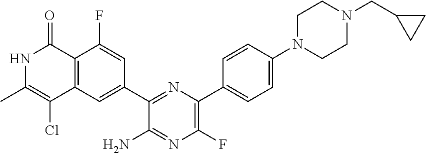 [SMILES: Cc1[nH]c(=O)c2c(F)cc(-c3nc(-c4ccc(N5CCN(CC6CC6)CC5)cc4)c(F)nc3N)cc2c1Cl]

Prepared from 6-(3-amino-6-bromo-5-fluoropyrazin-2-yl)-4-chloro-8-fluoro-3-methylisoquinolin-1(2H)-one (35 mg, 0.087 mmol) and 1-(cyclopropylmethyl)-4-(4-(4,4,5,5-tetramethyl-1,3,2-dioxaborolan-2-yl)phenyl)piperazine (35.8 mg, 0.105 mmol) to give the title compound (9.6 mg, 20.5% yield) as a yellow solid. ¹H NMR (500 MHz, DMSO-d₆) δ ppm 11.71 (br. s., 1H), 7.98 (s, 1H), 7.75 (d, J=8.19 Hz, 2H), 7.57 (d, J=11.98 Hz, 1H), 7.03 (d, J=8.93 Hz, 2H), 6.89 (s, 2H), 3.19-3.25 (m, 4H), 2.56-2.60 (m, 4H), 2.37 (s, 3H), 2.23 (d, J=6.48 Hz, 2H), 0.82-0.90 (m, 1H), 0.46-0.52 (m, 2H), 0.10 (q, J=4.77 Hz, 2H); LCMS: [M+H]⁺=537.46.

### Example 108: 6′-(3-amino-6-(4-(4-(cyclopropylmethyl)piperazin-1-yl)phenyl)-5-fluoropyrazin-2-yl)-2′,3′-dihydro-1′H-spiro[cyclopropane-1,4′-isoquinolin]-1′-one (I-108)

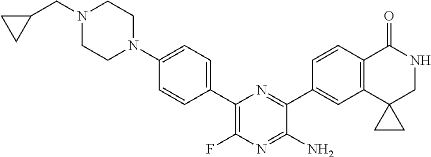 [SMILES: Nc1nc(F)c(-c2ccc(N3CCN(CC4CC4)CC3)cc2)nc1-c1ccc2c(c1)C1(CC1)CNC2=O]

Prepared from 6′-(3-amino-6-bromo-5-fluoropyrazin-2-yl)-2′,3′-dihydro-1′H-spiro[cyclopropane-1,4′-isoquinolin]-1′-one (35 mg, 0.096 mmol) and 1-(cyclopropylmethyl)-4-(4-(4,4,5,5-tetramethyl-1,3,2-dioxaborolan-2-yl)phenyl)piperazine (39.6 mg, 0.116 mmol) to give the title compound (9.9 mg, 20.6% yield) as a yellow solid. ¹H NMR (500 MHz, DMSO-d₆) δ ppm 7.97 (br. s., 1H), 7.89 (d, J=7.95 Hz, 1H), 7.63 (d, J=8.19 Hz, 2H), 7.53 (d, J=7.95 Hz, 1H), 7.17 (s, 1H), 6.91 (d, J=8.68 Hz, 2H), 6.56 (br. s., 2H), 3.16-3.18 (m, 2H), 3.09-3.13 (m, 4H), 2.45-2.49 (m, 4H), 2.12 (d, J=6.48 Hz, 2H), 1.01-1.06 (m, 2H), 0.87-0.92 (m, 2H), 0.75 (m, J=5.40 Hz, 1H), 0.38 (m, J=7.30 Hz, 2H), 0.00 (m, J=4.30 Hz, 2H); LCMS: [M+H]⁺=499.55.

### Example 109: 6-(3-amino-6-(3-((dimethylamino)methyl)-4-morpholinophenyl)-5-fluoropyrazin-2-yl)-4-chloro-8-fluoro-3-methylisoquinolin-1(2H)-one (I-109)

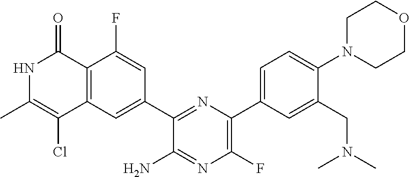 [SMILES: Cc1[nH]c(=O)c2c(F)cc(-c3nc(-c4ccc(N5CCOCC5)c(CN(C)C)c4)c(F)nc3N)cc2c1Cl]

Prepared from 6-(3-amino-6-bromo-5-fluoropyrazin-2-yl)-4-chloro-8-fluoro-3-methylisoquinolin-1(2H)-one (40 mg, 0.100 mmol) and N,N-dimethyl-1-(2-morpholino-5-(4,4,5,5-tetramethyl-1,3,2-dioxaborolan-2-yl)phenyl)methanamine (41.4 mg, 0.120 mmol) to give the title compound (15.2 mg, 28.2% yield) as a yellow solid. ¹H NMR (500 MHz, DMSO-d₆) δ ppm 11.71 (br. s., 1H), 8.00 (s, 1H), 7.91 (br. s., 1H), 7.74 (d, J=8.31 Hz, 1H), 7.58 (d, J=12.23 Hz, 1H), 7.36 (br. s., 2H), 7.17 (d, J=8.44 Hz, 1H), 7.01 (br. s., 1H), 3.75 (br. s., 4H), 3.46 (br. s., 2H), 2.96 (br. s., 4H), 2.37 (s, 3H), 2.19 (br. s., 6H); LCMS: [M+H]⁺=541.54.

### Example 110: 6′-(3-amino-6-(3-((dimethylamino)methyl)-4-(tetrahydro-2H-pyran-4-yl)phenyl)-5-fluoropyrazin-2-yl)-2′,3′-dihydro-1′H-spiro[cyclopropane-1,4′-isoquinolin]-1′-one (I-110)

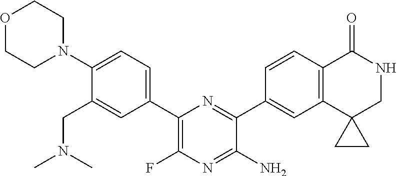 [SMILES: CN(C)Cc1cc(-c2nc(-c3ccc4c(c3)C3(CC3)CNC4=O)c(N)nc2F)ccc1N1CCOCC1]

Prepared from 6′-(3-amino-6-bromo-5-fluoropyrazin-2-yl)-2′,3′-dihydro-1′H-spiro[cyclopropane-1,4′-isoquinolin]-1′-one (40 mg, 0.110 mmol) and N,N-dimethyl-1-(2-(tetrahydro-2H-pyran-4-yl)-5-(4,4,5,5-tetramethyl-1,3,2-dioxaborolan-2-yl)phenyl)methanamine (45.6 mg, 0.132 mmol) to give the title compound (14.5 mg, 26.2% yield) as a yellow solid. ¹H NMR (500 MHz, DMSO-d₆) δ ppm 8.08 (br. s., 1H), 7.99 (d, J=7.95 Hz, 1H), 7.72 (d, J=1.34 Hz, 2H), 7.63 (dd, J=8.01, 1.53 Hz, 1H), 7.37-7.41 (m, 1H), 7.28 (d, J=1.34 Hz, 1H), 6.82 (s, 2H), 3.93-3.99 (m, 2H), 3.42-3.47 (m, 4H), 3.27 (d, J=2.57 Hz, 2H), 3.16-3.24 (m, 1H), 2.17 (s, 6H), 1.71 (qd, J=12.27, 4.16 Hz, 2H), 1.59-1.65 (m, 2H), 1.11-1.16 (m, 2H), 0.98-1.02 (m, 2H); LCMS: [M+H]⁺=502.49.

### Example 111: 6-(3-amino-5-fluoro-6-(4-(4-isopropylpiperazin-1-yl)phenyl)pyrazin-2-yl)-4,8-difluoro-3-methylisoquinolin-1(2H)-one (I-111)

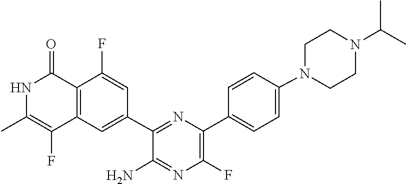 [SMILES: Cc1[nH]c(=O)c2c(F)cc(-c3nc(-c4ccc(N5CCN(C(C)C)CC5)cc4)c(F)nc3N)cc2c1F]

### Step 1: 6-bromo-4,8-difluoro-3-methylisoquinolin-1(2H)-one

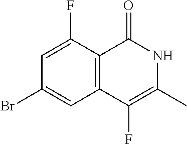

A vial was charged with 6-bromo-8-fluoro-3-methylisoquinolin-1(2H)-one (1.13 g, 4.41 mmol) and Selectfluor™ fluorinating reagent >95% in F+ active (1.641 g, 4.63 mmol). Methanol (10 ml) and acetonitrile (10 ml) were added, and the reaction was stirred at room temperature for 5 days. The white suspension was filtered, washed with water, and dried under vacuum to afford the product. LCMS: [M+H]⁺=274.14.

### Step 2: 4,8-difluoro-3-methyl-1-oxo-1,2-dihydroisoquinolin-6-yl)boronic acid

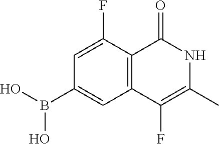 [SMILES: Cc1[nH]c(=O)c2c(F)cc(B(O)O)cc2c1F]

Prepared from 6-bromo-4,8-difluoro-3-methylisoquinolin-1(2H)-one (458 mg, 1.671 mmol) to give a mixture of the boronate and boronic acid which was used in the next step without further purification. LCMS: [M+H]⁺=240.26 Boronic acid, 322.25 Boronate.

### Step 3: 6-(3-amino-6-bromo-5-fluoropyrazin-2-yl)-4,8-difluoro-3-methylisoquinolin-1(2H)-one

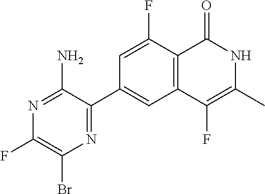 [SMILES: Cc1[nH]c(=O)c2c(F)cc(-c3nc(Br)c(F)nc3N)cc2c1F]

Prepared from 5-bromo-6-fluoro-3-iodopyrazin-2-amine (220 mg, 0.692 mmol) and (4,8-difluoro-3-methyl-1-oxo-1,2-dihydroisoquinolin-6-yl)boronic acid (198 mg, 0.830 mmol) to give the product (153 mg, 57.4% yield) as a yellow solid. LCMS: [M+H]⁺=385.16.

### Step 4: 6-(3-amino-5-fluoro-6-(4-(4-isopropylpiperazin-1-yl)phenyl)pyrazin-2-yl)-4,8-difluoro-3-methylisoquinolin-1(2H)-one

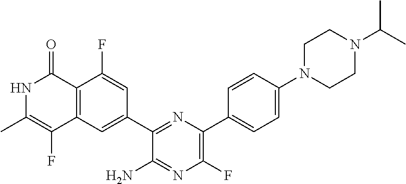 [SMILES: Cc1[nH]c(=O)c2c(F)cc(-c3nc(-c4ccc(N5CCN(C(C)C)CC5)cc4)c(F)nc3N)cc2c1F]

Prepared from 6-(3-amino-6-bromo-5-fluoropyrazin-2-yl)-4,8-difluoro-3-methylisoquinolin-1(2H)-one (35 mg, 0.091 mmol) and 4-(4-isopropylpiperazinyl)phenylboronic acid, pinacol ester (36.0 mg, 0.109 mmol) to give the title compound (14.6 mg, 31.6% yield) as a yellow solid. ¹H NMR (500 MHz, DMSO-d₆) δ ppm 11.30 (br. s., 1H), 7.77 (s, 1H), 7.75 (d, J=7.95 Hz, 2H), 7.56 (d, J=12.35 Hz, 1H), 7.02 (d, J=9.05 Hz, 2H), 6.91 (s, 2H), 3.17-3.22 (m, 4H), 2.67 (dt, J=13.08, 6.54 Hz, 1H), 2.56-2.61 (m, 4H), 2.23 (d, J=2.93 Hz, 3H), 1.01 (d, J=6.60 Hz, 6H); LCMS: [M+H]⁺=509.51.

### Example 112: 6-(3-amino-5-fluoro-6-(4-morpholino-3-(pyrrolidin-1-ylmethyl)phenyl)pyrazin-2-yl)-8-fluoro-3,4-dihydroisoquinolin-1(2H)-one (I-112)

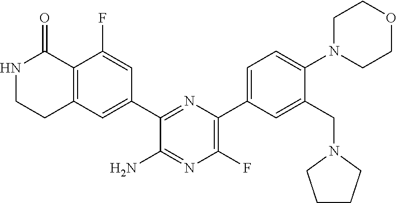 [SMILES: Nc1nc(F)c(-c2ccc(N3CCOCC3)c(CN3CCCC3)c2)nc1-c1cc(F)c2c(c1)CCNC2=O]

### Step 1: 4-(4-bromo-2-(pyrrolidin-1-ylmethyl)phenyl)morpholine

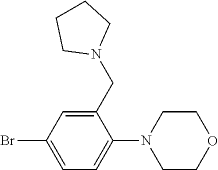 [SMILES: Brc1ccc(N2CCOCC2)c(CN2CCCC2)c1]

Prepared from 5-bromo-2-morpholinobenzaldehyde (400 mg, 1.481 mmol) commercially available with pyrrolidine (0.486 mL, 5.92 mmol). The title compound was isolated as a yellow solid (508 mg, 95% yield based on 90% purity). LCMS: [M+H]⁺=325.29

### Step 2: 4-(2-(pyrrolidin-1-ylmethyl)-4-(4,4,5,5-tetramethyl-1,3,2-dioxaborolan-2-yl)phenyl)morpholine

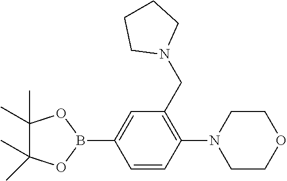 [SMILES: CC1(C)OB(c2ccc(N3CCOCC3)c(CN3CCCC3)c2)OC1(C)C]

Prepared from 4-(4-bromo-2-(pyrrolidin-1-ylmethyl)phenyl)morpholine (180 mg, 0.553 mmol) to give the product as a brown solid which was taken to the next step without any purification. (210 mg, 97% yield based on 95% purity); LCMS: [M+H]⁺=373.56

### Step 3: 6-(3-amino-5-fluoro-6-(4-morpholino-3-(pyrrolidin-1-ylmethyl)phenyl)pyrazin-2-yl)-8-fluoro-3,4-dihydroisoquinolin-1(2H)-one

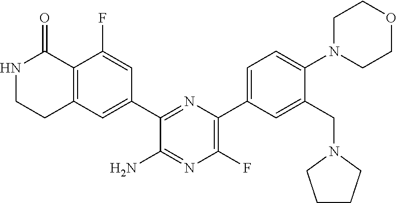 [SMILES: Nc1nc(F)c(-c2ccc(N3CCOCC3)c(CN3CCCC3)c2)nc1-c1cc(F)c2c(c1)CCNC2=O]

Prepared from 6-(3-amino-6-bromo-5-fluoropyrazin-2-yl)-8-fluoro-3,4-dihydroisoquinolin-1(2H)-one (50 mg, 0.169 mmol) and 4-(2-(pyrrolidin-1-ylmethyl)-4-(4,4,5,5-tetramethyl-1,3-dioxolan-2-yl)phenyl)morpholine (66.6 mg, 0.179 mmol) to give the title compound as a beige solid (33 mg, 43% yield). ¹H NMR (500 MHz, DMSO-d6) δ ppm 8.04 (br. s., 1H), 7.91 (br. s., 1H), 7.72 (d, J=8.07 Hz, 1H), 7.50 (s, 1H), 7.43 (d, J=11.98 Hz, 1H), 7.16 (d, J=8.31 Hz, 1H), 6.93 (br. s., 2H), 3.76 (br. s., 4H), 3.67 (br. s., 2H), 3.30 (d, J=1.59 Hz, 2H), 2.97 (br. s., 6H), 2.46-2.50 (m, 4H), 1.70 (br. s., 4H); LCMS: [M+H]⁺=521.61.

### Example 113: 6-(3-amino-5-fluoro-6-(4-morpholino-3-(pyrrolidin-1-ylmethyl)phenyl)pyrazin-2-yl)-3,4-dihydroisoquinolin-1(2H)-one (I-113)

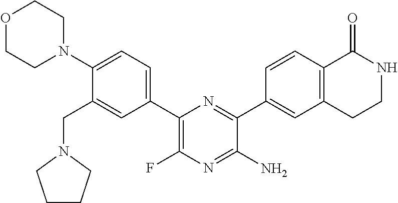 [SMILES: Nc1nc(F)c(-c2ccc(N3CCOCC3)c(CN3CCCC3)c2)nc1-c1ccc2c(c1)CCNC2=O]

Prepared from 4-(2-(pyrrolidin-1-ylmethyl)-4-(4,4,5,5-tetramethyl-1,3-dioxolan-2-yl)phenyl)morpholine (66.7 mg, 0.178 mmol) and 4-(2-(pyrrolidin-1-ylmethyl)-4-(4,4,5,5-tetramethyl-1,3-dioxolan-2-yl)phenyl)morpholine (66.7 mg, 0.178 mmol) to give the title compound as a beige solid (32 mg, 41% yield). ¹H NMR (500 MHz, DMSO-d₆) δ ppm 7.99 (br. s., 1H), 7.95 (d, J=8.07 Hz, 1H), 7.91 (s, 1H), 7.73-7.73 (m, 1H), 7.71 (dd, J=16.02, 8.31 Hz, 2H), 7.65 (s, 1H), 7.16 (d, J=8.44 Hz, 1H), 6.83 (s, 2H), 3.75 (d, J=4.03 Hz, 4H), 3.67 (s, 2H), 3.42-3.46 (m, 2H), 2.90-3.00 (m, 6H), 2.42-2.49 (m, 4H), 1.70 (br. s., 4H); LCMS: [M+H]⁺=503.56

### Example 114: 6-(3-amino-5-fluoro-6-(4-morpholino-3-(pyrrolidin-1-ylmethyl)phenyl)pyrazin-2-yl)isoquinolin-1(2H)-one (I-114)

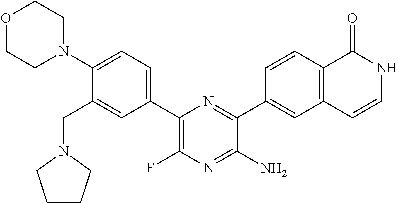 [SMILES: Nc1nc(F)c(-c2ccc(N3CCOCC3)c(CN3CCCC3)c2)nc1-c1ccc2c(=O)[nH]ccc2c1]

Prepared from 6-(3-amino-6-bromo-5-fluoropyrazin-2-yl)isoquinolin-1(2H)-one (50 mg, 0.149 mmol) and 4-(2-(pyrrolidin-1-ylmethyl)-4-(4,4,5,5-tetramethyl-1,3-dioxolan-2-yl)phenyl)morpholine (67.1 mg, 0.179 mmol) to give the title compound as a beige solid (29 mg, 37% yield). ¹H NMR (500 MHz, DMSO-d₆) δ ppm 11.29 (d, J=4.77 Hz, 1H), 8.24-8.33 (m, 1H), 7.99 (s, 1H), 7.89-7.95 (m, 1H), 7.81 (d, J=8.44 Hz, 1H), 7.74 (d, J=8.44 Hz, 1H), 7.23 (t, J=6.42 Hz, 1H), 7.16 (d, J=8.44 Hz, 1H), 6.82-6.96 (m, 2H), 6.60-6.67 (m, 1H), 3.75 (d, J=4.03 Hz, 4H), 3.67 (s, 2H), 2.97 (br. s., 4H), 2.52-2.54 (m, 4H), 1.69 (br. s., 4H); LCMS: [M+H]⁺=501.55.

### Example 115: 6-(3-amino-6-(4-(4-(cyclopropylmethyl)piperazin-1-yl)phenyl)-5-fluoropyrazin-2-yl)-4,8-difluoro-3-methylisoquinolin-1(2H)-one (I-115)

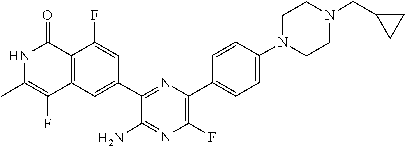 [SMILES: Cc1[nH]c(=O)c2c(F)cc(-c3nc(-c4ccc(N5CCN(CC6CC6)CC5)cc4)c(F)nc3N)cc2c1F]

Prepared from 6-(3-amino-6-bromo-5-fluoropyrazin-2-yl)-4,8-difluoro-3-methylisoquinolin-1(2H)-one (35 mg, 0.091 mmol) and 1-(cyclopropylmethyl)-4-(4-(4,4,5,5-tetramethyl-1,3,2-dioxaborolan-2-yl)phenyl)piperazine (37.3 mg, 0.109 mmol) to give the title compound (18.9 mg, 40.0% yield) as a yellow solid. ¹H NMR (500 MHz, DMSO-d₆) δ ppm 11.30 (br. s., 1H), 7.73-7.78 (m, 2H), 7.53-7.58 (m, 1H), 7.03 (d, J=8.93 Hz, 1H), 6.91 (s, 2H), 6.72-6.80 (m, 1H), 6.60-6.67 (m, 1H), 3.21-3.23 (m, 2H), 2.92-2.99 (m, 2H), 2.57-2.59 (m, 2H), 2.53-2.56 (m, 2H), 2.23 (d, J=2.32 Hz, 3H), 2.19-2.22 (m, 2H), 0.80-0.90 (m, 1H), 0.45-0.50 (m, 2H), 0.08-0.12 (m, 2H); LCMS: [M+H]⁺=521.45.

### Example 116: 6-(3-amino-6-(3-((dimethylamino)methyl)-4-morpholinophenyl)-5-fluoropyrazin-2-yl)-4,8-difluoro-3-methylisoquinolin-1(2H)-one (I-116)

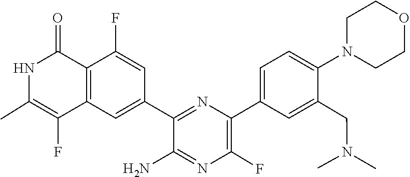 [SMILES: Cc1[nH]c(=O)c2c(F)cc(-c3nc(-c4ccc(N5CCOCC5)c(CN(C)C)c4)c(F)nc3N)cc2c1F]

Prepared from 6-(3-amino-6-bromo-5-fluoropyrazin-2-yl)-4,8-difluoro-3-methylisoquinolin-1(2H)-one (35 mg, 0.091 mmol) and N,N-dimethyl-1-(2-morpholino-5-(4,4,5,5-tetramethyl-1,3,2-dioxaborolan-2-yl)phenyl)methanamine (37.8 mg, 0.109 mmol) to give the product (11.4 mg, 23.9% yield) as a yellow solid. ¹H NMR (500 MHz, DMSO-d₆) b ppm 11.31 (br. s., 1H), 7.89 (s, 1H), 7.77 (s, 1H), 7.74 (d, J=8.44 Hz, 1H), 7.55 (d, J=12.35 Hz, 1H), 7.17 (d, J=8.56 Hz, 1H), 7.01 (s, 2H), 3.73-3.78 (m, 4H), 3.46 (s, 2H), 2.92-2.99 (m, 4H), 2.23 (d, J=2.81 Hz, 3H), 2.19 (s, 6H); LCMS: [M+H]⁺=525.44.

### Example 117: 6-(3-amino-6-(3-((dimethylamino)methyl)-4-(tetrahydro-2H-pyran-4-yl)phenyl)-5-fluoropyrazin-2-yl)-4,8-difluoro-3-methylisoquinolin-1(2H)-one (I-117)

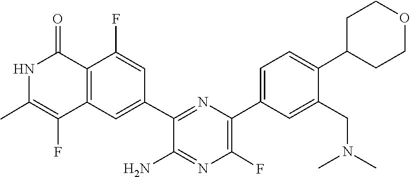 [SMILES: Cc1[nH]c(=O)c2c(F)cc(-c3nc(-c4ccc(C5CCOCC5)c(CN(C)C)c4)c(F)nc3N)cc2c1F]

Prepared from 6-(3-amino-6-bromo-5-fluoropyrazin-2-yl)-4,8-difluoro-3-methylisoquinolin-1(2H)-one (35 mg, 0.091 mmol) and N,N-dimethyl-1-(2-(tetrahydro-2H-pyran-4-yl)-5-(4,4,5,5-tetramethyl-1,3,2-dioxaborolan-2-yl)phenyl)methanamine (37.7 mg, 0.109 mmol) to give the title compound (10.2 mg, 21.4% yield) as a yellow solid. ¹H NMR (500 MHz, DMSO-d₆) δ ppm 11.31 (br. s., 1H), 7.70-7.79 (m, 3H), 7.55 (d, J=12.35 Hz, 1H), 7.41 (d, J=8.07 Hz, 1H), 7.05 (s, 2H), 3.96 (dd, J=10.88, 3.30 Hz, 2H), 3.43-3.48 (m, 4H), 3.17-3.25 (m, 1H), 2.23 (d, J=2.81 Hz, 3H), 2.17 (s, 6H), 1.72 (qd, J=12.25, 3.97 Hz, 2H), 1.59-1.66 (m, 2H); LCMS: [M+H]⁺=524.46.

### Example 118: 6-(3-amino-5-fluoro-6-(3-((4-methoxypiperidin-1-yl)methyl)-4-morpholinophenyl)pyrazin-2-yl)-7-fluoro-3,4-dihydroisoquinolin-1(2H)-one (I-118)

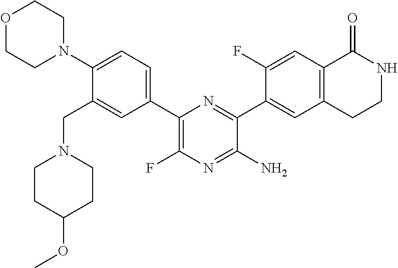 [SMILES: COC1CCN(Cc2cc(-c3nc(-c4cc5c(cc4F)C(=O)NCC5)c(N)nc3F)ccc2N2CCOCC2)CC1]

Prepared from 6-(3-amino-6-bromo-5-fluoropyrazin-2-yl)-7-fluoro-3,4-dihydroisoquinolin-1(2H)-one (0.030 g, 0.084 mmol) and 4-(2-((4-methoxypiperidin-1-yl)methyl)-4-(4,4,5,5-tetramethyl-1,3,2-dioxaborolan-2-yl)phenyl)morpholine (0.053 g, 0.117 mmol) to give the title compound as a beige solid (7 mg, 14% yield). ¹H NMR (500 MHz, DMSO-d₆) δ=8.06-7.97 (m, 1H), 7.87-7.75 (m, 2H), 7.64-7.50 (m, 3H), 7.28-7.20 (m, 1H), 3.93-3.82 (m, 4H), 3.73-3.64 (m, 2H), 3.57 (br t, J=6.4 Hz, 2H), 3.40-3.35 (m, 3H), 3.28-3.23 (m, 1H), 3.11-3.05 (m, 2H), 3.04-2.96 (m, 4H), 2.92-2.81 (m, 2H), 2.41-2.20 (m, 2H), 1.98-1.88 (m, 2H), 1.56 (br d, J=8.7 Hz, 2H; LCMS: [M+H]⁺=565.46.

### Example 119: 6-(3-amino-5-fluoro-6-(4-(4-isopropylpiperazin-1-yl)phenyl)pyrazin-2-yl)-4-methoxyisoquinolin-1(2H)-one (I-119)

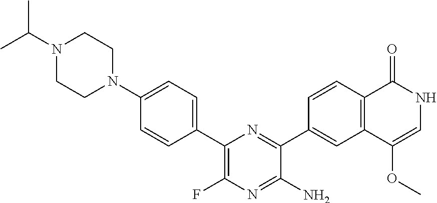 [SMILES: COc1c[nH]c(=O)c2ccc(-c3nc(-c4ccc(N5CCN(C(C)C)CC5)cc4)c(F)nc3N)cc12]

### Step 1: 6-bromo-4-methoxyisoquinolin-1(2H)-one

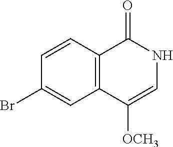 [SMILES: COc1c[nH]c(=O)c2ccc(Br)cc12]

A vial was charged with 6-bromo-2H-isoquinolin-1-one (300 mg, 1.339 mmol), methanol (2 ml) and methanesulfonic acid (0.087 ml, 1.339 mmol) then cooled to 0° C. A solution of iodobenzene diacetate (474 mg, 1.473 mmol) in methanol (2 ml) was added slowly. The reaction was warmed to room temperature and stirred for 1 hour then heated at 50° C. overnight. The reaction was concentrated in vacuo and triturated with isopropanol. The filtered solid was vacuum dried to afford the product (257 mg, 76% yield) as a pink solid. LCMS: [M+H]⁺=254.20.

### Step 2: 4-methoxy-6-(4,4,5,5-tetramethyl-1,3,2-dioxaborolan-2-yl)isoquinolin-1(2H)-one

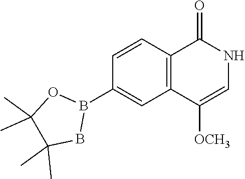

Prepared from 6-bromo-4-methoxyisoquinolin-1(2H)-one (70 mg, 0.276 mmol) to give the boronate which was used in the next step without further purification. LCMS: [M+H]+=302.23.

### Step 3: 6-(3-amino-5-fluoro-6-(4-(4-isopropylpiperazin-1-yl)phenyl)pyrazin-2-yl)-4-methoxyisoquinolin-1(2H)-one

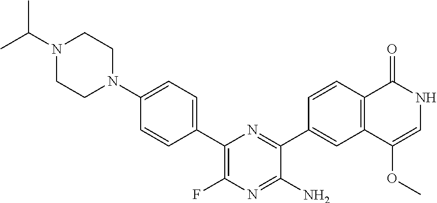 [SMILES: COc1c[nH]c(=O)c2ccc(-c3nc(-c4ccc(N5CCN(C(C)C)CC5)cc4)c(F)nc3N)cc12]

Prepared from 4-methoxy-6-(4,4,5,5-tetramethyl-1,3,2-dioxaborolan-2-yl)isoquinolin-1(2H)-one (36.7 mg, 0.122 mmol) and 3-bromo-6-fluoro-5-(4-(4-isopropylpiperazin-1-yl)phenyl)pyrazin-2-amine (40 mg, 0.101 mmol) to give the product (25.1 mg, 50.6% yield) as a bright yellow solid. ¹H NMR (500 MHz, DMSO-d₆) δ ppm 10.99 (d, J=5.38 Hz, 1H), 8.29 (d, J=8.31 Hz, 1H), 8.17 (d, J=1.34 Hz, 1H), 7.89 (dd, J=8.31, 1.59 Hz, 1H), 7.74 (d, J=7.95 Hz, 2H), 7.01 (d, J=9.05 Hz, 2H), 6.78 (d, J=5.75 Hz, 1H), 6.76 (s, 2H), 3.81 (s, 3H), 3.17-3.21 (m, 4H), 2.67 (dt, J=13.02, 6.57 Hz, 1H), 2.56-2.60 (m, 4H), 1.00 (d, J=6.48 Hz, 6H); LCMS: [M+H]⁺=489.56.

### Example 120: 6-(3-amino-6-(3-((dimethylamino)methyl)-4-morpholinophenyl)-5-fluoropyrazin-2-yl)-4,8-difluoroisoquinolin-1(2H)-one (I-120)

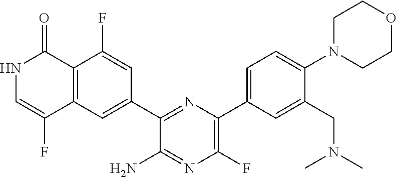 [SMILES: CN(C)Cc1cc(-c2nc(-c3cc(F)c4c(=O)[nH]cc(F)c4c3)c(N)nc2F)ccc1N1CCOCC1]

### Step 1: 6-(3-amino-6-bromo-5-fluoropyrazin-2-yl)-4,8-difluoroisoquinolin-1(2H)-one

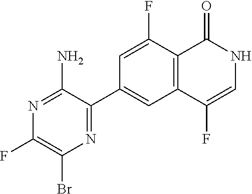 [SMILES: Nc1nc(F)c(Br)nc1-c1cc(F)c2c(=O)[nH]cc(F)c2c1]

Prepared from 5-bromo-6-fluoro-3-iodopyrazin-2-amine (178 mg, 0.560 mmol) and (4,8-difluoro-1-oxo-1,2-dihydroisoquinolin-6-yl)boronic acid (151 mg, 0.672 mmol) to give the product (103 mg, 49.6% yield) as a beige solid. LCMS: [M+H]⁺=371.24.

### Step 2: 6-(3-amino-6-(3-((dimethylamino)methyl)-4-morpholinophenyl)-5-fluoropyrazin-2-yl)-4,8-difluoroisoquinolin-1(2H)-one

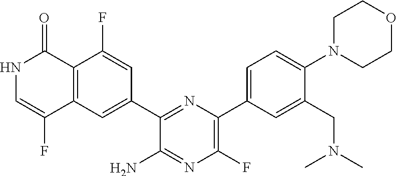 [SMILES: CN(C)Cc1cc(-c2nc(-c3cc(F)c4c(=O)[nH]cc(F)c4c3)c(N)nc2F)ccc1N1CCOCC1]

Prepared from 6-(3-amino-6-bromo-5-fluoropyrazin-2-yl)-4,8-difluoroisoquinolin-1(2H)-one (33 mg, 0.089 mmol) and N,N-dimethyl-1-(2-morpholino-5-(4,4,5,5-tetramethyl-1,3,2-dioxaborolan-2-yl)phenyl)methanamine (36.9 mg, 0.107 mmol) to give the title compound (10.5 mg, 23.1% yield) as a yellow solid. ¹H NMR (500 MHz, DMSO-d₆) δ ppm 11.19 (br. s., 1H), 7.89 (s, 1H), 7.84 (s, 1H), 7.74 (d, J=8.44 Hz, 1H), 7.64 (d, J=12.35 Hz, 1H), 7.49 (d, J=5.87 Hz, 1H), 7.18 (d, J=8.44 Hz, 1H), 7.04 (s, 2H), 3.73-3.77 (m, 4H), 3.46 (s, 2H), 2.93-2.98 (m, 4H), 2.19 (s, 6H); LCMS [M+H]⁺=511.37.

### Example 121: 6-(3-amino-6-(3-((dimethylamino)methyl)-4-(tetrahydro-2H-pyran-4-yl)phenyl)-5-fluoropyrazin-2-yl)-4,8-difluoroisoquinolin-1(2H)-one (I-121)

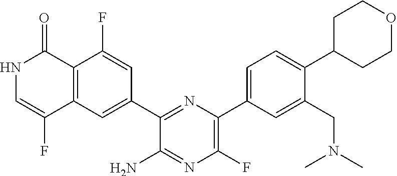 [SMILES: CN(C)Cc1cc(-c2nc(-c3cc(F)c4c(=O)[nH]cc(F)c4c3)c(N)nc2F)ccc1C1CCOCC1]

Prepared from 6-(3-amino-6-bromo-5-fluoropyrazin-2-yl)-4,8-difluoroisoquinolin-1(2H)-one (33 mg, 0.089 mmol) and N,N-dimethyl-1-(2-(tetrahydro-2H-pyran-4-yl)-5-(4,4,5,5-tetramethyl-1,3,2-dioxaborolan-2-yl)phenyl)methanamine (36.8 mg, 0.107 mmol) to give the title compound (12.5 mg, 27.6% yield) as a yellow solid. ¹H NMR (500 MHz, DMSO-d₆) δ ppm 11.21 (br. s., 1H), 7.84 (s, 1H), 7.75 (d, J=8.19 Hz, 1H), 7.73 (s, 1H), 7.64 (d, J=12.35 Hz, 1H), 7.49 (d, J=5.75 Hz, 1H), 7.41 (d, J=8.19 Hz, 1H), 7.08 (s, 2H), 3.96 (dd, J=10.64, 3.67 Hz, 2H), 3.42-3.48 (m, 4H), 3.18-3.25 (m, 1H), 2.17 (s, 6H), 1.72 (qd, J=12.25, 3.97 Hz, 2H), 1.59-1.66 (m, 2H); LCMS: [M+H]⁺=510.47.

### Example 122: 6-(3-amino-6-(4-(4-(cyclopropylmethyl)piperazin-1-yl)phenyl)-5-fluoropyrazin-2-yl)-4-chloro-3-methylisoquinolin-1(2H)-one (I-122)

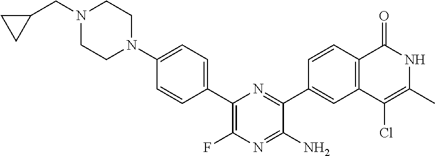 [SMILES: Cc1[nH]c(=O)c2ccc(-c3nc(-c4ccc(N5CCN(CC6CC6)CC5)cc4)c(F)nc3N)cc2c1Cl]

Prepared from 6-(3-amino-6-bromo-5-fluoropyrazin-2-yl)-4-chloro-3-methylisoquinolin-1(2H)-one (27 mg, 0.070 mmol) and 1-(cyclopropylmethyl)-4-(4-(4,4,5,5-tetramethyl-1,3,2-dioxaborolan-2-yl)phenyl)piperazine (28.9 mg, 0.084 mmol) to give the title compound (13.9 mg, 38.0% yield) as a yellow solid. ¹H NMR (500 MHz, DMSO-d₆) δ ppm 11.71 (br. s., 1H), 8.30 (d, J=8.19 Hz, 1H), 8.16 (d, J=1.22 Hz, 1H), 7.87 (dd, J=8.25, 1.41 Hz, 1H), 7.75 (d, J=7.82 Hz, 2H), 7.03 (d, J=9.05 Hz, 2H), 6.81 (s, 2H), 3.19-3.24 (m, 4H), 2.56-2.60 (m, 4H), 2.39 (s, 3H), 2.23 (d, J=6.60 Hz, 2H), 0.82-0.90 (m, 1H), 0.46-0.50 (m, 2H), 0.07-0.12 (m, 2H); LCMS: [M+H]⁺=519.57.

### Example 123: 6-(3-amino-5-fluoro-6-(4-(8-methyl-5-oxa-2,8-diazaspiro[3.5]nonan-2-yl)phenyl)pyrazin-2-yl)-4-fluoro-3-methylisoquinolin-1(2H)-one (I-123)

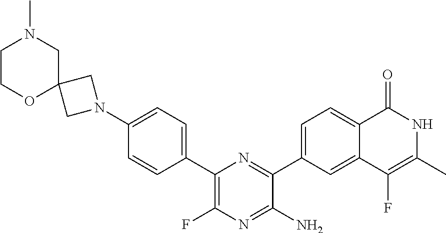 [SMILES: Cc1[nH]c(=O)c2ccc(-c3nc(-c4ccc(N5CC6(CN(C)CCO6)C5)cc4)c(F)nc3N)cc2c1F]

Prepared from 6-(3-amino-6-bromo-5-fluoropyrazin-2-yl)-4-fluoro-3-methylisoquinolin-1(2H)-one (31.1 mg, 0.085 mmol) reacted with 8-methyl-2-(4-(4,4,5,5-tetramethyl-1,3,2-dioxaborolan-2-yl)phenyl)-5-oxa-2,8-diazaspiro[3.5]nonane (40 mg, 0.085 mmol) to give the title compound (8.5 mg, 19.5% yield). ¹H NMR (500 MHz, METHANOL-d₄) b=8.28 (dd, J=1.6, 8.3 Hz, 1H), 8.05 (s, 1H), 7.86 (dd, J=1.5, 8.4 Hz, 1H), 7.73 (d, J=7.6 Hz, 2H), 6.48 (d, J=8.8 Hz, 2H), 3.81 (d, J=8.1 Hz, 2H), 3.67-3.57 (m, 4H), 2.55 (br s, 2H), 2.38-2.31 (m, 2H), 2.27-2.20 (m, 6H); LCMS: [M+H]⁺=505.50.

### Example 124: 6-(3-amino-6-(3-((dimethylamino)methyl)-4-morpholinophenyl)-5-fluoropyrazin-2-yl)-4-chloro-3-methylisoquinolin-1(2H)-one (I-124)

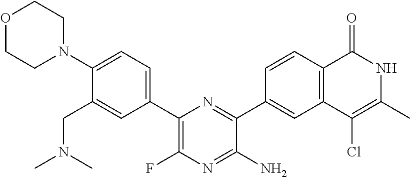 [SMILES: Cc1[nH]c(=O)c2ccc(-c3nc(-c4ccc(N5CCOCC5)c(CN(C)C)c4)c(F)nc3N)cc2c1Cl]

Prepared from 6-(3-amino-6-bromo-5-fluoropyrazin-2-yl)-4-chloro-3-methylisoquinolin-1(2H)-one (27 mg, 0.070 mmol) and N,N-dimethyl-1-(2-morpholino-5-(4,4,5,5-tetramethyl-1,3,2-dioxaborolan-2-yl)phenyl)methanamine (29.2 mg, 0.084 mmol) to give the product (12.8 mg, 34.8% yield) as a yellow solid. ¹H NMR (500 MHz, DMSO-d₆) b ppm 11.72 (s, 1H), 8.30 (d, J=8.19 Hz, 1H), 8.17 (s, 1H), 7.91 (s, 1H), 7.88 (dd, J=8.25, 1.28 Hz, 1H), 7.74 (d, J=8.56 Hz, 1H), 7.17 (d, J=8.44 Hz, 1H), 6.92 (s, 2H), 3.73-3.77 (m, 4H), 3.46 (s, 2H), 2.94-2.97 (m, 4H), 2.39 (s, 3H), 2.19 (s, 6H); LCMS: [M+H]⁺=523.18.

### Example 125: 6-(3-amino-6-(3-((dimethylamino)methyl)-4-(tetrahydro-2H-pyran-4-yl)phenyl)-5-fluoropyrazin-2-yl)-4-chloro-3-methylisoquinolin-1(2H)-one (I-125)

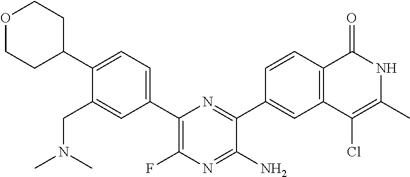 [SMILES: Cc1[nH]c(=O)c2ccc(-c3nc(-c4ccc(C5CCOCC5)c(CN(C)C)c4)c(F)nc3N)cc2c1Cl]

Prepared from 6-(3-amino-6-bromo-5-fluoropyrazin-2-yl)-4-chloro-3-methylisoquinolin-1(2H)-one (27 mg, 0.070 mmol) and N,N-dimethyl-1-(2-(tetrahydro-2H-pyran-4-yl)-5-(4,4,5,5-tetramethyl-1,3,2-dioxaborolan-2-yl)phenyl)methanamine (29.2 mg, 0.084 mmol) to give the title compound (14.3 mg, 38.9% yield) as a yellow solid. ¹H NMR (500 MHz, DMSO-d₆) δ ppm 11.72 (br. s., 1H), 8.31 (d, J=8.19 Hz, 1H), 8.17 (s, 1H), 7.87 (dd, J=8.25, 1.28 Hz, 1H), 7.75 (br. s., 2H), 7.38-7.43 (m, 1H), 6.96 (s, 2H), 3.96 (dd, J=10.82, 3.48 Hz, 2H), 3.42-3.47 (m, 4H), 3.17-3.25 (m, 1H), 2.39 (s, 3H), 2.17 (s, 6H), 1.72 (qd, J=12.25, 4.10 Hz, 2H), 1.59-1.66 (m, 2H); LCMS: [M+H]⁺=522.43.

### Example 126: 6-(3-amino-5-fluoro-6-(3-((4-methoxypiperidin-1-yl)methyl)-4-thiomorpholinophenyl)pyrazin-2-yl)-3,4-dihydroisoquinolin-1(2H)-one (I-264)

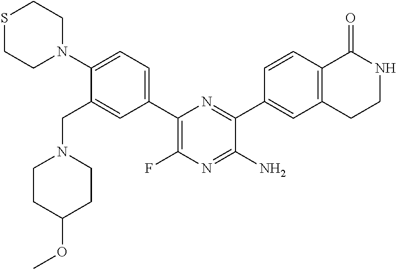 [SMILES: COC1CCN(Cc2cc(-c3nc(-c4ccc5c(c4)CCNC5=O)c(N)nc3F)ccc2N2CCSCC2)CC1]

### Step 1: 4-(4-bromo-2-((4-methoxypiperidin-1-yl)methyl)phenyl)thiomorpholine

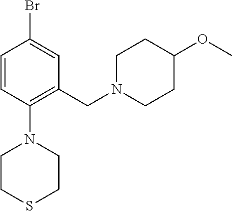 [SMILES: COC1CCN(Cc2cc(Br)ccc2N2CCSCC2)CC1]

Prepared from 5-bromo-2-thiomorpholinobenzaldehyde (177 mg, 0.618 mmol) and 4-methoxypiperidine (0.230 ml, 1.855 mmol) as the amine. The title compound was isolated as an off beige solid (259 mg, 98% based on 90% purity). LCMS: [M+H]⁺=385.41

### Step 2: 4-(2-((4-methoxypiperidin-1-yl)methyl)-4-(4,4,5,5-tetramethyl-1,3,2-dioxaborolan-2-yl)phenyl)thiomorpholine

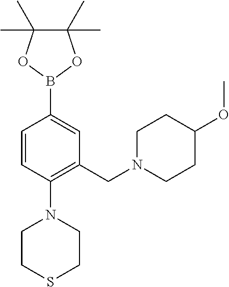

Prepared from 4-(4-bromo-2-((4-methoxypiperidin-1-yl)methyl)phenyl)thiomorpholine (120 mg, 0.280 mmol) to give the product as a brown solid which was taken to the next step without any purification. (121 mg, 95% yield based on 95% purity); LCMS: [M+H]⁺=433.61

### Step 3: 6-(3-amino-5-fluoro-6-(3-((4-methoxypiperidin-1-yl)methyl)-4-thiomorpholinophenyl)pyrazin-2-yl)-3,4-dihydroisoquinolin-1(2H)-one

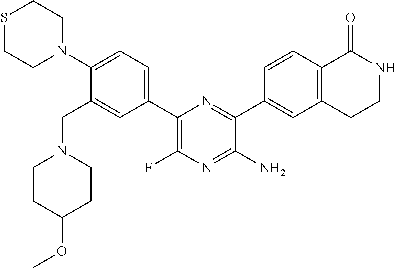 [SMILES: COC1CCN(Cc2cc(-c3nc(-c4ccc5c(c4)CCNC5=O)c(N)nc3F)ccc2N2CCSCC2)CC1]

Prepared from 6-(3-amino-6-bromo-5-fluoropyrazin-2-yl)-3,4-dihydroisoquinolin-1(2H)-one (0.039 g, 0.116 mmol) and 4-(2-((4-methoxypiperidin-1-yl)methyl)-4-(4,4,5,5-tetramethyl-1,3,2-dioxaborolan-2-yl)phenyl)thiomorpholine (0.060 g, 0.139 mmol) to give the title compound as a beige solid (45 mg, 66% yield). ¹H NMR (500 MHz, DMSO-d₆) δ ppm 8.00 (br. s., 1H), 7.95 (d, J=7.95 Hz, 1H), 7.92 (s, 1H), 7.69-7.76 (m, 2H), 7.65 (s, 1H), 7.18 (d, J=8.31 Hz, 1H), 6.80-6.92 (m, 2H), 3.50 (s, 2H), 3.41-3.46 (m, 2H), 3.22 (s, 3H), 3.17 (br. s., 5H), 2.99 (t, J=6.36 Hz, 2H), 2.77 (br. s., 4H), 2.72 (d, J=9.66 Hz, 2H), 2.14 (t, J=9.66 Hz, 2H), 1.83 (d, J=9.29 Hz, 2H), 1.36-1.45 (m, 2H); LCMS: [M+H]⁺=563.58

### Example 127: 6-(3-amino-5-fluoro-6-(3-((4-methoxypiperidin-1-yl)methyl)-4-thiomorpholinophenyl)pyrazin-2-yl)-7-fluoro-3,4-dihydroisoquinolin-1(2H)-one (I-127)

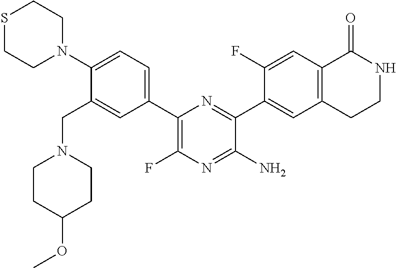 [SMILES: COC1CCN(Cc2cc(-c3nc(-c4cc5c(cc4F)C(=O)NCC5)c(N)nc3F)ccc2N2CCSCC2)CC1]

Prepared from 6-(3-amino-6-bromo-5-fluoropyrazin-2-yl)-7-fluoro-3,4-dihydroisoquinolin-1(2H)-one (0.041 g, 0.116 mmol) and 4-(2-((4-methoxypiperidin-1-yl)methyl)-4-(4,4,5,5-tetramethyl-1,3,2-dioxaborolan-2-yl)phenyl)thiomorpholine (0.060 g, 0.139 mmol) to give the title compound as a beige solid (30 mg, 42% yield). ¹H NMR (500 MHz, DMSO-d₆) δ=8.20-8.14 (m, 1H), 7.85 (s, 1H), 7.71-7.62 (m, 2H), 7.54-7.50 (m, 1H), 7.20-7.14 (m, 1H), 6.82 (br s, 2H), 3.52-3.46 (m, 2H), 3.44-3.39 (m, 2H), 3.24-3.20 (m, 3H), 3.16 (br s, 5H), 2.95 (br t, J=6.1 Hz, 2H), 2.80-2.74 (m, 4H), 2.73-2.67 (m, 2H), 2.17-2.09 (m, 2H), 1.85-1.78 (m, 2H), 1.43-1.35 (m, 2H); LCMS: [M+H]⁺=581.41

### Example 128: 6-(3-amino-6-(3-((dimethylamino)methyl)-4-thiomorpholinophenyl)-5-fluoropyrazin-2-yl)-3,4-dihydroisoquinolin-1(2H)-one (I-128)

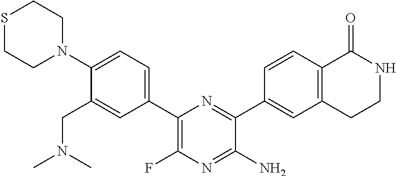 [SMILES: CN(C)Cc1cc(-c2nc(-c3ccc4c(c3)CCNC4=O)c(N)nc2F)ccc1N1CCSCC1]

### Step 1: 4-(2-(azetidin-1-ylmethyl)-4-bromo-3-fluorophenyl)morpholine

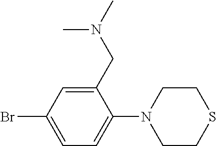 [SMILES: CN(C)Cc1cc(Br)ccc1N1CCSCC1]

Prepared from 5-bromo-2-thiomorpholinobenzaldehyde (2.305 g, 8.05 mmol) and dimethylamine, 2.0 M solution in THF (16.11 mL, 32.2 mmol). The product was isolated as an beige solid (2.51 g, 99%). LCMS: [M+H]⁺=315.27

### Step 2: N,N-dimethyl-1-(5-(4,4,5,5-tetramethyl-1,3,2-dioxaborolan-2-yl)-2-thiomorpholinophenyl)methanamine

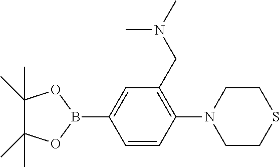 [SMILES: CN(C)Cc1cc(B2OC(C)(C)C(C)(C)O2)ccc1N1CCSCC1]

Prepared as per the procedure analogous to that of Example 1, step 1, using 1-(5-bromo-2-thiomorpholinophenyl)-N,N-dimethylmethanamine (140 mg, 0.444 mmol) Example 67, step 1. The product was isolated as a brown solid (161 mg, 95% yield based on 95% purity); LCMS: [M+H]⁺=363.25.

### Step 3: 6-(3-amino-6-(3-((dimethylamino)methyl)-4-thiomorpholinophenyl)-5-fluoropyrazin-2-yl)-3,4-dihydroisoquinolin-1(2H)-one

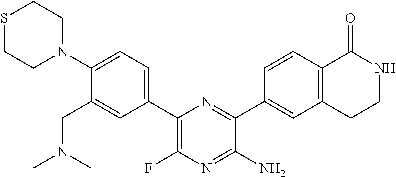 [SMILES: CN(C)Cc1cc(-c2nc(-c3ccc4c(c3)CCNC4=O)c(N)nc2F)ccc1N1CCSCC1]

Prepared from 6-(3-amino-6-bromo-5-fluoropyrazin-2-yl)-3,4-dihydroisoquinolin-1(2H)-one (60 mg, 0.178 mmol) and N,N-dimethyl-1-(5-(4,4,5,5-tetramethyl-1,3,2-dioxaborolan-2-yl)-2-thiomorpholinophenyl)methanamine (80 mg, 0.221 mmol) to give the title compound as a beige solid (42 mg, 45.5% yield). ¹H NMR (500 MHz, DMSO-d₆) δ=8.02-7.98 (m, 1H), 7.97-7.93 (m, 1H), 7.88-7.84 (m, 1H), 7.73 (br d, J=8.4 Hz, 1H), 7.67 (br d, J=8.1 Hz, 1H), 7.64 (s, 1H), 7.20-7.15 (m, 1H), 6.89-6.77 (m, 2H), 3.47-3.41 (m, 4H), 3.20-3.15 (m, 4H), 3.01-2.95 (m, 2H), 2.81-2.75 (m, 4H), 2.23-2.17 (m, 6H); LCMS: [M+H]⁺=493.53

### Example 129: 6-(3-amino-6-(3-((dimethylamino)methyl)-4-thiomorpholinophenyl)-5-fluoropyrazin-2-yl)-7-fluoro-3,4-dihydroisoquinolin-1(2H)-one (I-129)

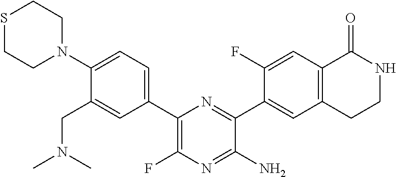 [SMILES: CN(C)Cc1cc(-c2nc(-c3cc4c(cc3F)C(=O)NCC4)c(N)nc2F)ccc1N1CCSCC1]

Prepared from 6-(3-amino-6-bromo-5-fluoropyrazin-2-yl)-7-fluoro-3,4-dihydroisoquinolin-1(2H)-one (60 mg, 0.169 mmol) and N,N-dimethyl-1-(5-(4,4,5,5-tetramethyl-1,3,2-dioxaborolan-2-yl)-2-thiomorpholinophenyl)methanamine (77 mg, 0.211 mmol) to give the title compound as an off white solid (39 mg, 43% yield). ¹H NMR (500 MHz, DMSO-d₆) δ=8.21-8.13 (m, 1H), 7.86-7.79 (m, 1H), 7.71-7.60 (m, 2H), 7.56-7.48 (m, 1H), 7.21-7.14 (m, 1H), 6.91-6.68 (m, 2H), 3.43 (s, 4H), 3.23-3.12 (m, 4H), 2.99-2.93 (m, 2H), 2.77 (br d, J=3.8 Hz, 4H), 2.18 (s, 6H); LCMS: [M+H]⁺=511.52.

### Example 130: 7-(3-amino-5-fluoro-6-(4-(8-methyl-5-oxa-2,8-diazaspiro[3.5]nonan-2-yl)phenyl)pyrazin-2-yl)-2-methylquinazolin-4(3H)-one (I-130)

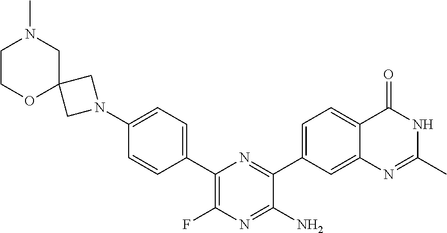 [SMILES: Cc1nc2cc(-c3nc(-c4ccc(N5CC6(CN(C)CCO6)C5)cc4)c(F)nc3N)ccc2c(=O)[nH]1]

Prepared from 7-(3-amino-6-bromo-5-fluoropyrazin-2-yl)-2-methylquinazolin-4(3H)-one (29.7 mg, 0.085 mmol) and 8-methyl-2-(4-(4,4,5,5-tetramethyl-1,3,2-dioxaborolan-2-yl)phenyl)-5-oxa-2,8-diazaspiro[3.5]nonane (40 mg, 0.085 mmol) to give the title compound (8.0 mg, 18.6% yield). ¹H NMR (500 MHz, DMSO-d₆) δ=12.26 (br s, 1H), 8.16 (d, J=8.2 Hz, 1H), 7.90 (s, 1H), 7.80 (d, J=7.9 Hz, 1H), 7.73 (br d, J=7.8 Hz, 2H), 6.78-6.72 (m, 2H), 6.55 (d, J=8.6 Hz, 2H), 3.89-3.79 (m, 2H), 3.71-3.56 (m, 4H), 2.54-2.47 (m, 2H), 2.38 (s, 3H), 2.29 (br s, 2H), 2.21 (br s, 3H); LCMS: [M+H]⁺=488.45.

### Example 131: 6-(3-amino-5-fluoro-6-(3-((4-methoxypiperidin-1-yl)methyl)-4-(tetrahydro-2H-pyran-4-yl)phenyl)pyrazin-2-yl)-3,4-dihydroisoquinolin-1(2H)-one (I-131)

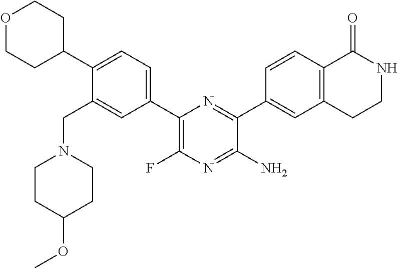 [SMILES: COC1CCN(Cc2cc(-c3nc(-c4ccc5c(c4)CCNC5=O)c(N)nc3F)ccc2C2CCOCC2)CC1]

Prepared from 6-(3-amino-6-bromo-5-fluoropyrazin-2-yl)-3,4-dihydroisoquinolin-1(2H)-one (55 mg, 0.163 mmol) and 4-methoxy-1-(2-(tetrahydro-2H-pyran-4-yl)-5-(4,4,5,5-tetramethyl-1,3,2-dioxaborolan-2-yl)benzyl)piperidine (81 mg, 0.196 mmol, in turn prepared from a sequence starting from 5-bromo-2-(tetrahydro-2H-pyran-4-yl)benzaldehyde and 4-methoxypiperidine using methods similar to those described earlier) to give the title compound as an off white solid (30, 32% yield). ¹H NMR (DMSO-d₆, 500 MHz) δ ppm 8.00 (br. s., 1H), 7.96 (d, J=7.95 Hz, 1H), 7.77 (s, 1H), 7.68-7.75 (m, 2H), 7.66 (s, 1H), 7.37 (d, J=8.19 Hz, 1H), 6.88 (s, 2H), 3.93-4.02 (m, 3H), 3.71 (s, 2H), 3.45-3.53 (m, 4H), 3.41-3.45 (m, 2H), 3.15 (s, 3H), 3.11 (t, J=11.68 Hz, 1H), 2.99 (t, J=6.36 Hz, 2H), 2.89 (t, J=6.48 Hz, 2H), 1.67-1.76 (m, 2H), 1.59-1.66 (m, 2H); LCMS: [M+H]⁺=518.47.

### Example 132: 6-(3-amino-5-fluoro-6-(3-((4-methoxypiperidin-1-yl)methyl)-4-(tetrahydro-2H-pyran-4-yl)phenyl)pyrazin-2-yl)-7-fluoro-3,4-dihydroisoquinolin-1(2H)-one (I-132)

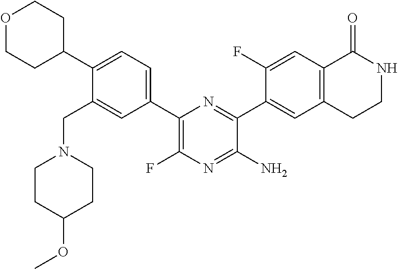 [SMILES: COC1CCN(Cc2cc(-c3nc(-c4cc5c(cc4F)C(=O)NCC5)c(N)nc3F)ccc2C2CCOCC2)CC1]

Prepared from 6-(3-amino-6-bromo-5-fluoropyrazin-2-yl)-7-fluoro-3,4-dihydroisoquinolin-1(2H)-one (55 mg, 0.155 mmol) and 4-methoxy-1-(2-(tetrahydro-2H-pyran-4-yl)-5-(4,4,5,5-tetramethyl-1,3,2-dioxaborolan-2-yl)benzyl)piperidine (77 mg, 0.186 mmol) to give the title compound as an off white solid (44, 48% yield). ¹H NMR (500 MHz, DMSO-d₆) δ ppm 8.17 (br. s., 1H), 7.70 (d, J=8.19 Hz, 1H), 7.62-7.68 (m, 2H), 7.52 (d, J=6.85 Hz, 1H), 7.38 (d, J=8.31 Hz, 1H), 6.85 (br. s., 2H), 3.97 (dd, J=10.70, 3.12 Hz, 2H), 3.51 (s, 2H), 3.43 (dd, J=6.72, 3.67 Hz, 4H), 3.13-3.26 (m, 5H), 2.96 (t, J=6.30 Hz, 2H), 2.64 (d, J=1.47 Hz, 2H), 2.10 (t, J=9.60 Hz, 2H), 1.80 (d, J=9.41 Hz, 2H), 1.72 (qd, J=12.15, 4.16 Hz, 2H), 1.59-1.67 (m, 2H), 1.29-1.41 (m, 2H); LCMS: [M+H]⁺=564.54

### Example 133: 6-(3-amino-5-fluoro-6-(3-((3-methoxypyrrolidin-1-yl)methyl)-4-morpholinophenyl)pyrazin-2-yl)-3,4-dihydroisoquinolin-1(2H)-one (I-133)

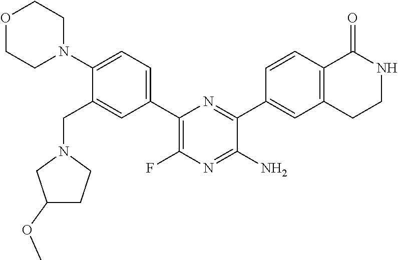 [SMILES: COC1CCN(Cc2cc(-c3nc(-c4ccc5c(c4)CCNC5=O)c(N)nc3F)ccc2N2CCOCC2)C1]

Prepared from 6-(3-amino-6-bromo-5-fluoropyrazin-2-yl)-3,4-dihydroisoquinolin-1(2H)-one (60 mg, 0.178 mmol) and 4-(2-((3-methoxypyrrolidin-1-yl)methyl)-4-(4,4,5,5-tetramethyl-1,3,2-dioxaborolan-2-yl)phenyl)morpholine (90 mg, 0.222 mmol, in turn prepared from a sequence starting from 5-bromo-2-morpholinobenzaldehyde and 3-methoxypyrrolidine analogous to previous examples) to give the title compound as a beige solid (47 mg, 47% yield). 1H NMR (500 MHz, DMSO-d6) δ=8.04-7.98 (m, 1H), 7.97-7.92 (m, 1H), 7.89 (s, 1H), 7.75-7.68 (m, 2H), 7.67-7.62 (m, 1H), 7.20-7.13 (m, 1H), 6.91-6.78 (m, 2H), 3.91-3.86 (m, 1H), 3.80-3.73 (m, 4H), 3.66 (s, 2H), 3.46-3.41 (m, 2H), 3.17-3.13 (m, 3H), 3.02-2.94 (m, 6H), 2.75-2.70 (m, 1H), 2.66-2.60 (m, 2H), 2.47-2.44 (m, 1H), 2.05-1.94 (m, 1H), 1.69-1.61 (m, 1H); LCMS: [M+H]⁺=533.52.

### Example 134: 6-(3-amino-5-fluoro-6-(3-((3-methoxypyrrolidin-1-yl)methyl)-4-morpholinophenyl)pyrazin-2-yl)-7-fluoro-3,4-dihydroisoquinolin-1(2H)-one (I-134)

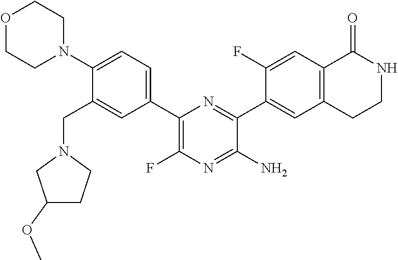 [SMILES: COC1CCN(Cc2cc(-c3nc(-c4cc5c(cc4F)C(=O)NCC5)c(N)nc3F)ccc2N2CCOCC2)C1]

Prepared from 6-(3-amino-6-bromo-5-fluoropyrazin-2-yl)-7-fluoro-3,4-dihydroisoquinolin-1(2H)-one (60 mg, 0.169 mmol) and 4-(2-((3-methoxypyrrolidin-1-yl)methyl)-4-(4,4,5,5-tetramethyl-1,3,2-dioxaborolan-2-yl)phenyl)morpholine (85 mg, 0.211 mmol) to give the title compound as a beige solid (43 mg, 44% yield). ¹H NMR (500 MHz, DMSO-d₆) δ 8.1-8.2 (m, 1H), 7.8-7.9 (m, 1H), 7.68 (br d, 1H, J=8.4 Hz), 7.6-7.7 (m, 1H), 7.5-7.5 (m, 1H), 7.1-7.2 (m, 1H), 6.8-6.9 (m, 2H), 3.8-3.9 (m, 1H), 3.75 (br s, 4H), 3.6-3.7 (m, 2H), 3.4-3.5 (m, 2H), 3.13 (s, 3H), 2.95 (br s, 6H), 2.7-2.7 (m, 1H), 2.6-2.7 (m, 2H), 2.4-2.5 (m, 1H), 1.9-2.0 (m, 1H), 1.6-1.7 (m, 1H); LCMS: [M+H]⁺=551.56

### Example 135: 6-(3-amino-5-fluoro-6-(4-((1S,5R)-3-methyl-3-azabicyclo[3.1.0]hexan-1-yl)phenyl)pyrazin-2-yl)-4,4,8-trifluoro-3-methyl-3,4-dihydroisoquinolin-1(2H)-one (I-135)

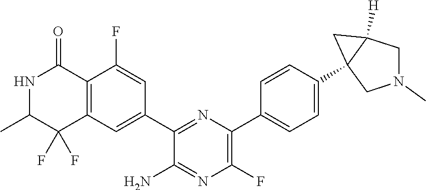

### Step 1: 6-bromo-4,4,8-trifluoro-3-hydroxy-3-methyl-3,4-dihydroisoquinolin-1(2H)-one

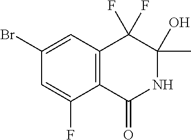 [SMILES: CC1(O)NC(=O)c2c(F)cc(Br)cc2C1(F)F]

6-Bromo-8-fluoro-3-methylisoquinolin-1(2H)-one (291 mg, 1.136 mmol) was mixed with acetonitrile (20 ml) at room temperature then Selectfluor™ (423 mg, 1.193 mmol) was added. The reaction mixture was stirred at room temperature for 3 days. Additional Selectfluor™ (200 mg, 0.565 mmol) was added and stirred at room temperature for another 4 days. Additional Selectfluor™ (100 mg, 0.282 mmol) was added and the reaction mixture was stirred at 50° C. for an hour, concentrated and diluted with water. The resulting precipitate was filtered, dried under air to give the product (310 mg, 88% yield) as an off-white solid. LCMS: [M+H]⁺=310.25.

### Step 2: 6-Bromo-4,4,8-trifluoro-3-methyl-3,4-dihydroisoquinolin-1(2H)-one

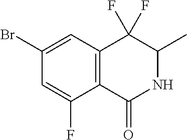 [SMILES: CC1NC(=O)c2c(F)cc(Br)cc2C1(F)F]

6-Bromo-4,4,8-trifluoro-3-hydroxy-3-methyl-3,4-dihydroisoquinolin-1(2H)-one (305 mg, 0.984 mmol) was mixed with dichloromethane (DCM) (6 ml) at RT, then TFA (0.753 mL, 9.84 mmol) and triethylsilane (0.314 ml, 1.967 mmol) were added. The reaction mixture was stirred at 45° C. for 5 hours and concentrated. The residue was diluted with water, and the resulting precipitate was filtered, rinsed with water and hexanes to give the product (256 mg, 89% yield) as an off-white solid. LCMS: [M+H]⁺=294.20.

### Step 3: 6-(3-amino-6-bromo-5-fluoropyrazin-2-yl)-4,4,8-trifluoro-3-methyl-3,4-dihydroisoquinolin-1(2H)-one

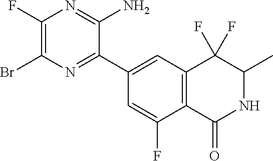 [SMILES: CC1NC(=O)c2c(F)cc(-c3nc(Br)c(F)nc3N)cc2C1(F)F]

6-(3-amino-6-bromo-5-fluoropyrazin-2-yl)-4,4,8-trifluoro-3-methyl-3,4-dihydroisoquinolin-1(2H)-one (137 mg, 38.9% yield) was prepared from 6-bromo-4,4,8-trifluoro-3-methyl-3,4-dihydroisoquinolin-1(2H)-one (255 mg, 0.867 mmol) reacted with potassium acetate (255 mg, 2.60 mmol) 1,1′-bis(diphenylphosphino)ferrocene-palladium(II)dichloride (63.4 mg, 0.087 mmol) and bis(pinacolato)diboron (242 mg 0.954 mmol) were mixed in 1,4-dioxane (10 ml) under vacuum and stirred under at 100° C. oil bath for an hour, followed by being reacted with 5-bromo-6-fluoro-3-iodopyrazin-2-amine (276 mg, 0.869 mmol) potassium carbonate (240 mg, 1.738 mmol) and tetrakis(triphenylphosphine)palladium(0) (100 mg, 0.087 mmol) in water (2 ml) under 85° C. oil bath overnight to give the product. LCMS: [M+H]⁺=405.28

### Step 4: 6-(3-amino-5-fluoro-6-(4-((1S,5R)-3-methyl-3-azabicyclo[3.1.0]hexan-1-yl)phenyl)pyrazin-2-yl)-4,4,8-trifluoro-3-methyl-3,4-dihydroisoquinolin-1(2H)-one

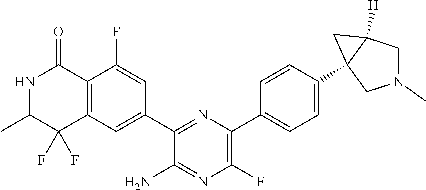

Prepared from 6-(3-amino-6-bromo-5-fluoropyrazin-2-yl)-4,4,8-trifluoro-3-methyl-3,4-dihydroisoquinolin-1(2H)-one (40.5 mg, 0.100 mmol) and (1S,5R)-3-methyl-1-(4-(4,4,5,5-tetramethyl-1,3,2-dioxaborolan-2-yl)phenyl)-3-azabicyclo[3.1.0]hexane (29.9 mg, 0.100 mmol) to give the title compound (8.35 mg, 15.3% yield). ¹H NMR (500 MHz, METHANOL-d₄) δ=7.89 (br d, J=8.1 Hz, 2H), 7.85 (s, 1H), 7.29 (d, J=8.3 Hz, 2H), 7.07 (d, J=8.4 Hz, 1H), 6.72 (d, J=8.4 Hz, 1H), 4.19-4.07 (m, 1H), 3.52 (br dd, J=9.7, 14.4 Hz, 2H), 3.03-2.90 (m, 2H), 2.86 (br dd, J=3.7, 9.5 Hz, 1H), 2.00-1.94 (m, 1H), 1.81 (td, J=4.0, 8.1 Hz, 1H), 1.45-1.38 (m, 3H), 1.21 (br t, J=4.2 Hz, 1H), 1.05-0.98 (m, 1H), 0.91 (br dd, J=5.7, 8.0 Hz, 1H) LCMS: [M+H]⁺=498.29.

### Example 136: 6-(3-amino-6-(4-(4-(4,4-difluorobutyl)piperazin-1-yl)phenyl)-5-fluoropyrazin-2-yl)-4-fluoroisoquinolin-1(2H)-one (I-136)

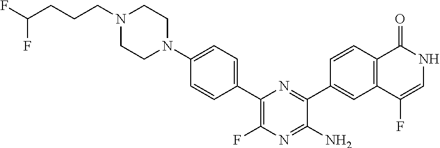 [SMILES: Nc1nc(F)c(-c2ccc(N3CCN(CCCC(F)F)CC3)cc2)nc1-c1ccc2c(=O)[nH]cc(F)c2c1]

### Step 1: 6-(3-amino-5-fluoro-6-(4-(piperazin-1-yl)phenyl)pyrazin-2-yl)-4-fluoroisoquinolin-1(2H)-one

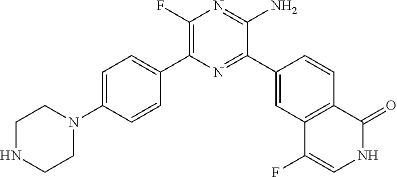 [SMILES: Nc1nc(F)c(-c2ccc(N3CCNCC3)cc2)nc1-c1ccc2c(=O)[nH]cc(F)c2c1]

Prepared from 6-(3-amino-6-bromo-5-fluoropyrazin-2-yl)-4-fluoroisoquinolin-1(2H)-one (50 mg, 0.142 mmol) and 4-piperazinylphenylboronic acid, pinacol ester (53.0 mg, 0.184 mmol) to give the product as a beige solid (42 mg, 61% yield); LCMS: [M+H]⁺=435.43.

### Step 2: 6-(3-amino-6-(4-(4-(4,4-difluorobutyl)piperazin-1-yl)phenyl)-5-fluoropyrazin-2-yl)-4-fluoroisoquinolin-1(2H)-one

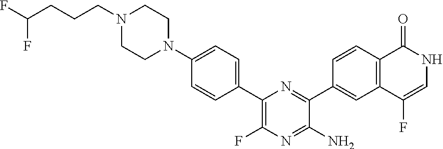 [SMILES: Nc1nc(F)c(-c2ccc(N3CCN(CCCC(F)F)CC3)cc2)nc1-c1ccc2c(=O)[nH]cc(F)c2c1]

Prepared from 6-(3-amino-5-fluoro-6-(4-(piperazin-1-yl)phenyl)pyrazin-2-yl)-4-fluoroisoquinolin-1(2H)-one (42 mg, 0.087 mmol) and 4-bromo-1,1-difluorobutane (30.1 mg, 0.174 mmol) to give the title compound as a yellow solid. (10 mg, 21% yield). 1H NMR (500 MHz, DMSO-d6) δ=11.28-11.12 (m, 1H), 8.39-8.23 (m, 1H), 8.11-8.03 (m, 1H), 8.00-7.92 (m, 1H), 7.82-7.69 (m, 2H), 7.50-7.38 (m, 1H), 7.09-7.00 (m, 2H), 6.93-6.80 (m, 2H), 6.25-5.99 (m, 1H), 3.24-3.16 (m, 4H), 2.55 (br s, 4H), 2.39-2.33 (m, 2H), 1.92-1.81 (m, 2H), 1.65-1.56 (m, 2H); LCMS: [M+H]⁺=527.56.

### Example 137: 6-(3-amino-6-(4-(4-(4,4-difluorobutyl)piperazin-1-yl)phenyl)-5-fluoropyrazin-2-yl)-8-fluoro-3,4-dihydroisoquinolin-1(2H)-one (I-137)

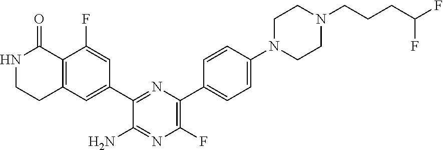 [SMILES: Nc1nc(F)c(-c2ccc(N3CCN(CCCC(F)F)CC3)cc2)nc1-c1cc(F)c2c(c1)CCNC2=O]

### Step 1: 6-(3-amino-5-fluoro-6-(4-(piperazin-1-yl)phenyl)pyrazin-2-yl)-4-fluoroisoquinolin-1(2H)-one

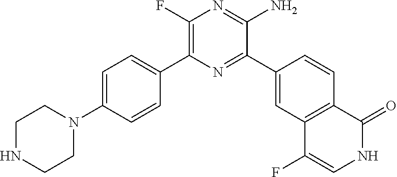 [SMILES: Nc1nc(F)c(-c2ccc(N3CCNCC3)cc2)nc1-c1ccc2c(=O)[nH]cc(F)c2c1]

Prepared from 6-(3-amino-6-bromo-5-fluoropyrazin-2-yl)-4-fluoroisoquinolin-1(2H)-one (50 mg, 0.142 mmol) and 4-piperazinylphenylboronic acid, pinacol ester (53.0 mg, 0.184 mmol) to give the product as a beige solid. (42 mg, 61% yield); LCMS: [M+H]⁺=435.43.

### Step 2: 6-(3-amino-6-(4-(4-(4,4-difluorobutyl)piperazin-1-yl)phenyl)-5-fluoropyrazin-2-yl)-4-fluoroisoquinolin-1(2H)-one

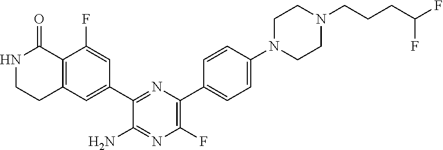 [SMILES: Nc1nc(F)c(-c2ccc(N3CCN(CCCC(F)F)CC3)cc2)nc1-c1cc(F)c2c(c1)CCNC2=O]

Prepared from 6-(3-amino-5-fluoro-6-(4-(piperazin-1-yl)phenyl)pyrazin-2-yl)-4-fluoroisoquinolin-1(2H)-one (42 mg, 0.087 mmol) and 4-bromo-1,1-difluorobutane (30.1 mg, 0.174 mmol) to give the title compound was as a yellow solid (10 mg, 21% yield). ¹H NMR (500 MHz, DMSO-d₆) δ=11.28-11.12 (m, 1H), 8.39-8.23 (m, 1H), 8.11-8.03 (m, 1H), 8.00-7.92 (m, 1H), 7.82-7.69 (m, 2H), 7.50-7.38 (m, 1H), 7.09-7.00 (m, 2H), 6.93-6.80 (m, 2H), 6.25-5.99 (m, 1H), 3.24-3.16 (m, 4H), 2.55 (br s, 4H), 2.39-2.33 (m, 2H), 1.92-1.81 (m, 2H), 1.65-1.56 (m, 2H); LCMS: [M+H]⁺=527.56.

### Example 138: 6-(3-amino-5-fluoro-6-(3-((4-methoxypiperidin-1-yl)methyl)-4-morpholinophenyl)pyrazin-2-yl)-3,4-dihydroisoquinolin-1(2H)-one (I-138)

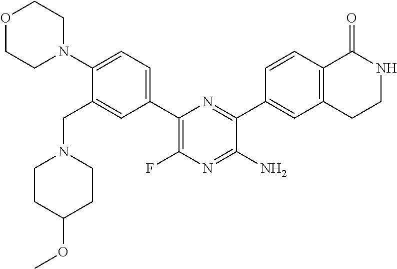 [SMILES: COC1CCN(Cc2cc(-c3nc(-c4ccc5c(c4)CCNC5=O)c(N)nc3F)ccc2N2CCOCC2)CC1]

Prepared from 6-(3-amino-6-bromo-5-fluoropyrazin-2-yl)-3,4-dihydroisoquinolin-1(2H)-one (48.6 mg, 0.144 mmol) and 4-(2-((4-methoxypiperidin-1-yl)methyl)-4-(4,4,5,5-tetramethyl-1,3,2-dioxaborolan-2-yl)phenyl)morpholine (79 mg, 0.180 mmol, in turn prepared from a sequence starting with 5-bromo-2-morpholinobenzaldehyde and 4-methoxypiperidine analogous to previous examples) to give the title compound as a pale-yellow solid (43 mg, 52% yield). ¹H NMR (500 MHz, DMSO-d₆) δ=8.04-7.98 (m, 1H), 7.97-7.90 (m, 2H), 7.76-7.68 (m, 2H), 7.68-7.62 (m, 1H), 7.22-7.14 (m, 1H), 6.92-6.80 (m, 2H), 3.81-3.71 (m, 4H), 3.58-3.51 (m, 2H), 3.47-3.41 (m, 2H), 3.25-3.20 (m, 3H), 3.20-3.15 (m, 1H), 3.03-2.97 (m, 2H), 2.97-2.90 (m, 4H), 2.77-2.69 (m, 2H), 2.21-2.10 (m, 2H), 1.83 (br d, J=9.9 Hz, 2H), 1.40 (br d, J=9.7 Hz, 2H); LCMS: [M+H]⁺=547.55.

### Example 139: 6-(3-amino-6-(4-(4-cyclobutylpiperazin-1-yl)phenyl)-5-fluoropyrazin-2-yl)-4-methylisoquinolin-1(2H)-one (I-139)

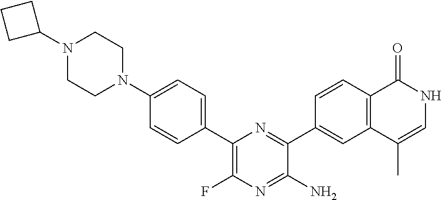 [SMILES: Cc1c[nH]c(=O)c2ccc(-c3nc(-c4ccc(N5CCN(C6CCC6)CC5)cc4)c(F)nc3N)cc12]

Prepared from 6 6-(3-amino-6-bromo-5-fluoropyrazin-2-yl)-4-methylisoquinolin-1(2H)-one (35 mg, 0.100 mmol) and 1-cyclobutyl-4-(4-(4,4,5,5-tetramethyl-1,3,2-dioxaborolan-2-yl)phenyl)piperazine (41.2 mg, 0.120 mmol) to give the title compound (31.5 mg, 64.8% yield) as a bright yellow solid. ¹H NMR (500 MHz, DMSO-d₆) b ppm 11.13 (d, J=5.50 Hz, 1H), 8.33 (d, J=8.31 Hz, 1H), 7.99 (s, 1H), 7.84 (d, J=8.31 Hz, 1H), 7.76 (d, J=8.19 Hz, 2H), 7.06 (d, J=5.38 Hz, 1H), 7.01 (d, J=9.05 Hz, 2H), 6.81 (s, 2H), 3.16-3.22 (m, 4H), 2.73 (quin, J=7.76 Hz, 1H), 2.36-2.41 (m, 4H), 2.26 (s, 3H), 1.95-2.03 (m, 2H), 1.77-1.86 (m, 2H), 1.60-1.70 (m, 2H); LCMS: [M+H]⁺=485.45.

### Example 140: 6-(3-amino-5-fluoro-6-(4-(4-isopropylpiperazin-1-yl)phenyl)pyrazin-2-yl)-4,7-difluoroisoquinolin-1(2H)-one (I-140)

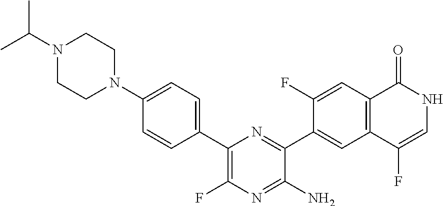 [SMILES: CC(C)N1CCN(c2ccc(-c3nc(-c4cc5c(F)c[nH]c(=O)c5cc4F)c(N)nc3F)cc2)CC1]

### Step 1: 6-bromo-4,7-difluoroisoquinolin-1(2H)-one

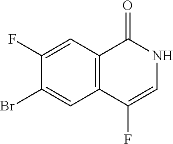 [SMILES: O=c1[nH]cc(F)c2cc(Br)c(F)cc12]

A RBF was charged with 6-bromo-7-fluoro-2H-isoquinolin-1-one (800 mg, 3.31 mmol) and Selectfluor™ fluorinating reagent (1405 mg, 3.97 mmol). Methanol (8 mL) and acetonitrile (8 ml) were added, and the reaction was heated to 50° C. and stirred for 1 h. The reaction was concentrated in vacuo, dissolved in 1,2-dichloroethane (16 ml), and phosphorous (V) oxychloride (0.618 ml, 6.61 mmol) was added. The reaction was stirred at 50° C. for 1h then quenched into ice water and extracted with DCM (2×). The combined organic layers were dried with anhydrous sodium sulfate and concentrated to afford the product (710 mg, 83%) as a white solid. LCMS: [M−H]⁻=258.09.

### Step 2: (4,7-difluoro-1-oxo-1,2-dihydroisoquinolin-6-yl)boronic acid

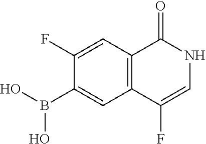 [SMILES: O=c1[nH]cc(F)c2cc(B(O)O)c(F)cc12]

Prepared from 6-bromo-4,7-difluoroisoquinolin-1(2H)-one (710 mg, 2.73 mmol) to give the boronic acid which was used in the next step without further purification. LCMS: [M+H]+=226.12.

### Step 3: 6-(3-amino-6-bromo-5-fluoropyrazin-2-yl)-4,7-difluoroisoquinolin-1(2H)-one

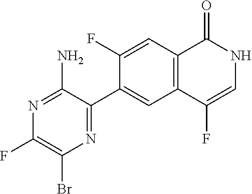 [SMILES: Nc1nc(F)c(Br)nc1-c1cc2c(F)c[nH]c(=O)c2cc1F]

Prepared from 5-bromo-6-fluoro-3-iodopyrazin-2-amine (353 mg, 1.110 mmol) and (4,7-difluoro-1-oxo-1,2-dihydroisoquinolin-6-yl)boronic acid (300 mg, 1.333 mmol) to give the product (281 mg, 68.2% yield) as a beige solid. ¹H NMR (500 MHz, DMSO-d₆) b ppm 11.36 (br. s., 1H), 7.99 (d, J=10.15 Hz, 1H), 7.88 (d, J=6.48 Hz, 1H), 7.45 (d, J=5.75 Hz, 1H), 7.07 (br. s., 2H); LCMS: [M+H]⁺=371.18.

### Step 4: 6-(3-amino-5-fluoro-6-(4-(4-isopropylpiperazin-1-yl)phenyl)pyrazin-2-yl)-4,7-difluoroisoquinolin-1(2H)-one

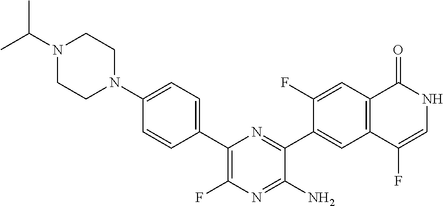 [SMILES: CC(C)N1CCN(c2ccc(-c3nc(-c4cc5c(F)c[nH]c(=O)c5cc4F)c(N)nc3F)cc2)CC1]

Prepared from 6-(3-amino-6-bromo-5-fluoropyrazin-2-yl)-4,7-difluoroisoquinolin-1(2H)-one (35 mg, 0.094 mmol) and 4-(4-isopropylpiperazinyl)phenylboronic acid, pinacol ester (37.4 mg, 0.113 mmol) to give the title compound (20.8 mg, 44.6% yield) as a bright yellow solid. ¹H NMR (500 MHz, DMSO-d₆) b ppm 11.33 (br. s., 1H), 8.00 (dd, J=10.03, 1.47 Hz, 1H), 7.92 (d, J=6.60 Hz, 1H), 7.70 (d, J=8.07 Hz, 2H), 7.44 (d, J=5.87 Hz, 1H), 6.99 (d, J=9.05 Hz, 2H), 6.78 (s, 2H), 3.14-3.20 (m, 4H), 2.64-2.69 (m, 1H), 2.55-2.58 (m, 4H), 1.00 (d, J=6.48 Hz, 6H); LCMS: [M+H]⁺=595.54.

### Example 141: 6-(3-amino-6-(4-(4-(cyclopropylmethyl)piperazin-1-yl)phenyl)-5-fluoropyrazin-2-yl)-4,7-difluoroisoquinolin-1(2H)-one (I-141)

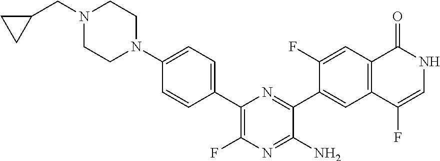 [SMILES: Nc1nc(F)c(-c2ccc(N3CCN(CC4CC4)CC3)cc2)nc1-c1cc2c(F)c[nH]c(=O)c2cc1F]

Prepared from 6-(3-amino-6-bromo-5-fluoropyrazin-2-yl)-4,7-difluoroisoquinolin-1(2H)-one (35 mg, 0.094 mmol) and 1-(cyclopropylmethyl)-4-(4-(4,4,5,5-tetramethyl-1,3,2-dioxaborolan-2-yl)phenyl)piperazine (38.7 mg, 0.113 mmol) to give the product (15.3 mg, 31.8% yield) as a yellow solid. ¹H NMR (500 MHz, DMSO-d₆) δ ppm 11.32 (br. s., 1H), 8.00 (dd, J=10.03, 1.59 Hz, 1H), 7.92 (d, J=6.60 Hz, 1H), 7.70 (d, J=7.83 Hz, 2H), 7.44 (d, J=5.87 Hz, 1H), 7.00 (d, J=8.93 Hz, 2H), 6.78 (s, 2H), 3.19-3.23 (m, 4H), 2.56-2.59 (m, 4H), 2.22 (d, J=6.60 Hz, 2H), 0.81-0.90 (m, 1H), 0.45-0.50 (m, 2H), 0.09 (q, J=4.81 Hz, 2H); LCMS: [M+H]⁺=507.51.

### Example 142: 6-(3-amino-6-(3-((dimethylamino)methyl)-4-morpholinophenyl)-5-fluoropyrazin-2-yl)-4,7-difluoroisoquinolin-1(2H)-one (I-142)

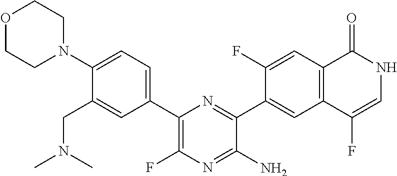 [SMILES: CN(C)Cc1cc(-c2nc(-c3cc4c(F)c[nH]c(=O)c4cc3F)c(N)nc2F)ccc1N1CCOCC1]

Prepared from 6-(3-amino-6-bromo-5-fluoropyrazin-2-yl)-4,7-difluoroisoquinolin-1(2H)-one (35 mg, 0.094 mmol) and N,N-dimethyl-1-(2-morpholino-5-(4,4,5,5-tetramethyl-1,3,2-dioxaborolan-2-yl)phenyl)methanamine (39.2 mg, 0.113 mmol) to give the title compound (12.3 mg, 25.5% yield) as a beige solid. ¹H NMR (500 MHz, DMSO-d₆) δ ppm 11.32 (br. s., 1H), 8.00 (dd, J=10.03, 1.59 Hz, 1H), 7.92 (d, J=6.60 Hz, 1H), 7.70 (d, J=7.83 Hz, 2H), 7.44 (d, J=5.87 Hz, 1H), 7.00 (d, J=8.93 Hz, 2H), 6.78 (s, 2H), 3.19-3.23 (m, 4H), 2.56-2.59 (m, 4H), 2.22 (d, J=6.60 Hz, 2H), 0.81-0.90 (m, 1H), 0.45-0.50 (m, 2H), 0.09 (q, J=4.81 Hz, 2H); LCMS: [M+H]⁺=511.52.

### Example 143: 6-(3-amino-6-(3-((dimethylamino)methyl)-4-(tetrahydro-2H-pyran-4-yl)phenyl)-5-fluoropyrazin-2-yl)-4,7-difluoroisoquinolin-1(2H)-one (I-143)

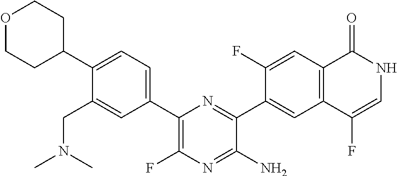 [SMILES: CN(C)Cc1cc(-c2nc(-c3cc4c(F)c[nH]c(=O)c4cc3F)c(N)nc2F)ccc1C1CCOCC1]

Prepared from 6-(3-amino-6-bromo-5-fluoropyrazin-2-yl)-4,7-difluoroisoquinolin-1(2H)-one (35 mg, 0.094 mmol) and N,N-dimethyl-1-(2-(tetrahydro-2H-pyran-4-yl)-5-(4,4,5,5-tetramethyl-1,3,2-dioxaborolan-2-yl)phenyl)methanamine (39.1 mg, 0.113 mmol) to give the title compound (22.4 mg, 46.6% yield) as a beige solid. ¹H NMR (500 MHz, DMSO-d₆) δ ppm 11.35 (br. s., 1H), 8.01 (dd, J=10.03, 1.47 Hz, 1H), 7.93 (d, J=6.48 Hz, 1H), 7.66-7.73 (m, 2H), 7.44 (d, J=5.87 Hz, 1H), 7.38 (d, J=8.19 Hz, 1H), 6.93 (s, 2H), 3.95 (dd, J=10.88, 3.42 Hz, 2H), 3.41-3.46 (m, 4H), 3.15-3.24 (m, 1H), 2.15 (s, 6H), 1.70 (qd, J=12.29, 4.10 Hz, 2H), 1.57-1.65 (m, 2H); LCMS: [M+H]⁺=510.51.

### Example 144: 6-(3-amino-6-(3-(azetidin-1-ylmethyl)-4-morpholinophenyl)-5-fluoropyrazin-2-yl)-3,4-dihydroisoquinolin-1(2H)-one (I-144)

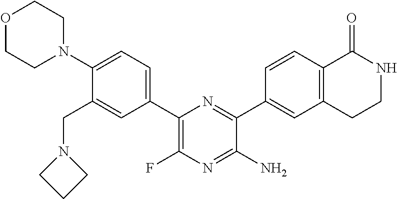 [SMILES: Nc1nc(F)c(-c2ccc(N3CCOCC3)c(CN3CCC3)c2)nc1-c1ccc2c(c1)CCNC2=O]

Prepared from 6-(3-amino-6-bromo-5-fluoropyrazin-2-yl)-3,4-dihydroisoquinolin-1(2H)-one and 4-(2-(azetidin-1-ylmethyl)-4-(4,4,5,5-tetramethyl-1,3,2-dioxaborolan-2-yl)phenyl)morpholine (69 mg, 0.183 mmol, in turn prepared from a sequence starting from 5-bromo-2-morpholinobenzaldehyde and azetidine) to give the title compound as a beige solid (35 mg, 45% yield). ¹H NMR (500 MHz, DMSO-d₆) δ=8.05-7.99 (m, 1H), 7.98-7.94 (m, 1H), 7.90-7.84 (m, 1H), 7.77-7.72 (m, 1H), 7.71-7.67 (m, 1H), 7.67-7.63 (m, 1H), 7.25-7.14 (m, 1H), 6.96-6.78 (m, 2H), 3.83-3.70 (m, 6H), 3.43 (br s, 2H), 3.31-3.28 (m, 4H), 3.05-2.97 (m, 2H), 2.92 (br s, 4H), 2.17-1.94 (m, 2H); LCMS: [M+H]⁺=489.46.

### Example 145: 6-(3-amino-6-(3-(azetidin-1-ylmethyl)-4-morpholinophenyl)-5-fluoropyrazin-2-yl)-7-fluoro-3,4-dihydroisoquinolin-1(2H)-one (I-145)

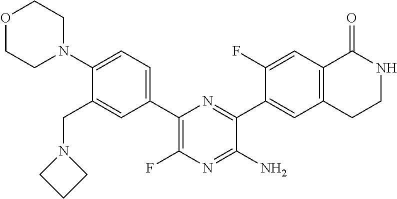 [SMILES: Nc1nc(F)c(-c2ccc(N3CCOCC3)c(CN3CCC3)c2)nc1-c1cc2c(cc1F)C(=O)NCC2]

Prepared from 6-(3-amino-6-bromo-5-fluoropyrazin-2-yl)-7-fluoro-3,4-dihydroisoquinolin-1(2H)-one (52.0 mg, 0.146 mmol) and 4-(2-(azetidin-1-ylmethyl)-4-(4,4,5,5-tetramethyl-1,3,2-dioxaborolan-2-yl)phenyl)morpholine (69 mg, 0.183 mmol) to give the title compound as a beige solid (40 mg, 51% yield). ¹H NMR (500 MHz, DMSO-d₆) δ=8.23-8.08 (m, 1H), 7.85-7.76 (m, 1H), 7.71-7.59 (m, 2H), 7.55-7.46 (m, 1H), 7.17-7.07 (m, 1H), 6.80 (br s, 2H), 3.81-3.72 (m, 4H), 3.64-3.57 (m, 2H), 3.46-3.41 (m, 2H), 3.15 (br t, J=6.7 Hz, 4H), 2.98-2.94 (m, 2H), 2.94-2.88 (m, 4H), 2.02-1.93 (m, 2H); LCMS: [M+H]⁺=507.57.

### Example 146: 6-(3-amino-6-(3-(azetidin-1-ylmethyl)-4-morpholinophenyl)-5-fluoropyrazin-2-yl)-8-fluoro-3,4-dihydroisoquinolin-1(2H)-one (I-146)

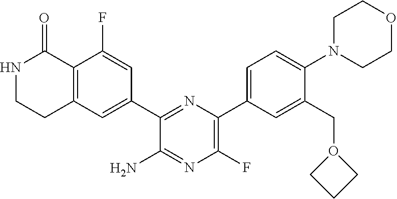

Prepared from 6-(3-amino-6-bromo-5-fluoropyrazin-2-yl)-8-fluoro-3,4-dihydroisoquinolin-1(2H)-one (52.0 mg, 0.146 mmol) PRML with 4-(2-(azetidin-1-ylmethyl)-4-(4,4,5,5-tetramethyl-1,3,2-dioxaborolan-2-yl)phenyl)morpholine (69 mg, 0.183 mmol) Example 25, step 2. The title compound was isolated as a pale-yellow solid (36 mg, 46% yield). ¹H NMR (500 MHz, DMSO-d6) δ=8.10-8.02 (m, 1H), 7.91-7.82 (m, 1H), 7.78-7.70 (m, 1H), 7.54-7.48 (m, 1H), 7.46-7.40 (m, 1H), 7.21-7.12 (m, 1H), 6.95 (br s, 2H), 3.80-3.74 (m, 4H), 3.74-3.65 (m, 2H), 3.38 (br s, 2H), 3.31 (br s, 4H), 3.03-2.96 (m, 2H), 2.91 (br s, 4H), 2.11-1.95 (m, 2H); LCMS: [M+H]⁺=507.57.

### Example 147: 6-(3-amino-5-fluoro-6-(3-(pyrrolidin-1-ylmethyl)-4-(tetrahydro-2H-pyran-4-yl)phenyl)pyrazin-2-yl)-3,4-dihydroisoquinolin-1(2H)-one (I-147)

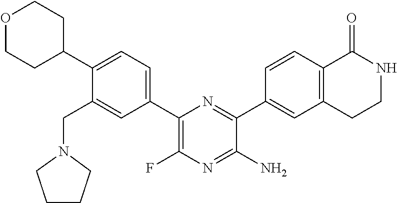 [SMILES: Nc1nc(F)c(-c2ccc(C3CCOCC3)c(CN3CCCC3)c2)nc1-c1ccc2c(c1)CCNC2=O]

### Step 1: 1-(2-(tetrahydro-2H-pyran-4-yl)-5-(4,4,5,5-tetramethyl-1,3,2-dioxaborolan-2-yl)benzyl)pyrrolidine

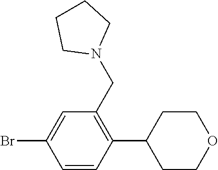 [SMILES: Brc1ccc(C2CCOCC2)c(CN2CCCC2)c1]

Prepared from 5-bromo-2-(tetrahydro-2H-pyran-4-yl)benzaldehyde (300 mg, 1.115 mmol) and pyrrolidine (159 mg, 2.229 mmol) to give the product (375 mg, 104% yield) as an orange oil which was carried onto the next step without further purification. LCMS: [M+H]⁺=324.42.

### Step 2: 1-(2-(tetrahydro-2H-pyran-4-yl)-5-(4,4,5,5-tetramethyl-1,3,2-dioxaborolan-2-yl)benzyl)pyrrolidine

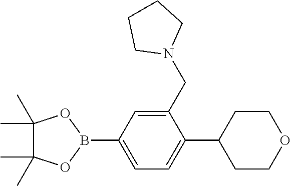 [SMILES: CC1(C)OB(c2ccc(C3CCOCC3)c(CN3CCCC3)c2)OC1(C)C]

Prepared from 1-(5-bromo-2-(tetrahydro-2H-pyran-4-yl)benzyl)pyrrolidine (80 mg, 0.247 mmol) to give the boronate which was used in the next step without further purification. LCMS [M+H]+ 372.42.

### Step 3: 6-(3-amino-5-fluoro-6-(3-(pyrrolidin-1-ylmethyl)-4-(tetrahydro-2H-pyran-4-yl)phenyl)pyrazin-2-yl)-3,4-dihydroisoquinolin-1(2H)-one (I-147)

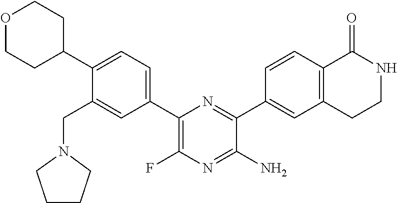 [SMILES: Nc1nc(F)c(-c2ccc(C3CCOCC3)c(CN3CCCC3)c2)nc1-c1ccc2c(c1)CCNC2=O]

Prepared from 6-(3-amino-6-bromo-5-fluoropyrazin-2-yl)-3,4-dihydroisoquinolin-1(2H)-one (35.7 mg, 0.106 mmol) and 1-(2-(tetrahydro-2H-pyran-4-yl)-5-(4,4,5,5-tetramethyl-1,3,2-dioxaborolan-2-yl)benzyl)pyrrolidine (55 mg, 0.138 mmol) to give the title compound as a brown solid (165 mg, 82% yield based on 82% purity). ¹H NMR (500 MHz, DMSO-d₆) δ=8.04-7.97 (m, 1H), 7.97-7.93 (m, 1H), 7.83-7.76 (m, 1H), 7.76-7.71 (m, 1H), 7.71-7.67 (m, 1H), 7.66-7.63 (m, 1H), 7.42-7.36 (m, 1H), 6.97-6.76 (m, 2H), 4.03-3.94 (m, 2H), 3.74-3.66 (m, 2H), 3.49-3.41 (m, 4H), 3.24-3.17 (m, 1H), 3.04-2.96 (m, 2H), 2.48-2.43 (m, 4H), 1.78-1.62 (m, 8H); LCMS: [M+H]⁺=502.62.

### Example 148: 6-(3-amino-5-fluoro-6-(3-(pyrrolidin-1-ylmethyl)-4-(tetrahydro-2H-pyran-4-yl)phenyl)pyrazin-2-yl)-7-fluoro-3,4-dihydroisoquinolin-1(2H)-one (I-148)

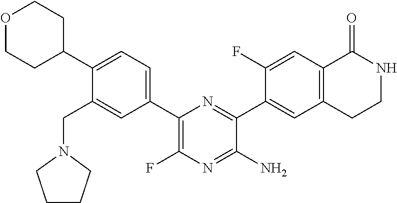 [SMILES: Nc1nc(F)c(-c2ccc(C3CCOCC3)c(CN3CCCC3)c2)nc1-c1cc2c(cc1F)C(=O)NCC2]

Prepared from 6-(3-amino-6-bromo-5-fluoropyrazin-2-yl)-7-fluoro-3,4-dihydroisoquinolin-1(2H)-one (37.6 mg, 0.106 mmol) and 1-(2-(tetrahydro-2H-pyran-4-yl)-5-(4,4,5,5-tetramethyl-1,3,2-dioxaborolan-2-yl)benzyl)pyrrolidine (55 mg, 0.138 mmol) to give the title compound as a white solid (28, 48% yield). ¹H NMR (500 MHz, DMSO-d₆) δ=8.20-8.13 (m, 1H), 7.76-7.71 (m, 1H), 7.71-7.67 (m, 1H), 7.65 (d, J=10.1 Hz, 1H), 7.55-7.50 (m, 1H), 7.40-7.35 (m, 1H), 6.83 (s, 2H), 4.00-3.94 (m, 2H), 3.71-3.65 (m, 2H), 3.48-3.41 (m, 4H), 3.25-3.18 (m, 1H), 2.99-2.93 (m, 2H), 2.45 (br s, 4H), 1.75-1.61 (m, 8H); LCMS: [M+H]⁺=520.54.

### Example 149: 6-(3-amino-5-fluoro-6-(3-(pyrrolidin-1-ylmethyl)-4-(tetrahydro-2H-pyran-4-yl)phenyl)pyrazin-2-yl)-8-fluoro-3,4-dihydroisoquinolin-1(2H)-one (I-149)

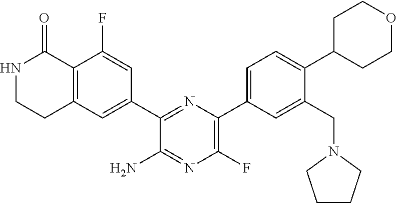 [SMILES: Nc1nc(F)c(-c2ccc(C3CCOCC3)c(CN3CCCC3)c2)nc1-c1cc(F)c2c(c1)CCNC2=O]

Prepared from 6-(3-amino-6-bromo-5-fluoropyrazin-2-yl)-8-fluoro-3,4-dihydroisoquinolin-1(2H)-one (37.6 mg, 0.106 mmol) and 1-(2-(tetrahydro-2H-pyran-4-yl)-5-(4,4,5,5-tetramethyl-1,3,2-dioxaborolan-2-yl)benzyl)pyrrolidine (55 mg, 0.138 mmol) to give the title compound as a pale-yellow solid (33, 57% yield). ¹H NMR (500 MHz, DMSO-d₆) δ=8.04 (br s, 1H), 7.82-7.76 (m, 1H), 7.75-7.69 (m, 1H), 7.53-7.48 (m, 1H), 7.46-7.41 (m, 1H), 7.41-7.36 (m, 1H), 7.07-6.89 (m, 2H), 4.01-3.95 (m, 2H), 3.69 (s, 2H), 3.45 (br t, J=11.0 Hz, 2H), 3.37 (br d, J=3.1 Hz, 2H), 3.23-3.17 (m, 1H), 3.00-2.95 (m, 2H), 2.48-2.44 (m, 4H), 1.77-1.63 (m, 8H); LCMS: [M+H]⁺=520.54

### Example 150: 6-(3-amino-6-(4-(4-cyclobutylpiperazin-1-yl)phenyl)-5-fluoropyrazin-2-yl)-4,4,8-trifluoro-3-methyl-3,4-dihydroisoquinolin-1(2H)-one (I-150)

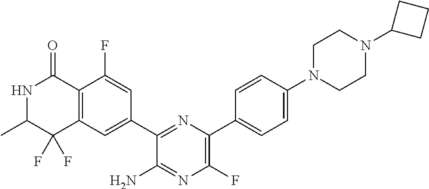 [SMILES: CC1NC(=O)c2c(F)cc(-c3nc(-c4ccc(N5CCN(C6CCC6)CC5)cc4)c(F)nc3N)cc2C1(F)F]

Prepared from 6-(3-amino-6-bromo-5-fluoropyrazin-2-yl)-4,4,8-trifluoro-3-methyl-3,4-dihydroisoquinolin-1(2H)-one (30 mg, 0.074 mmol) reacted with 1-cyclobutyl-4-(4-(4,4,5,5-tetramethyl-1,3,2-dioxaborolan-2-yl)phenyl)piperazine (25.3 mg, 0.074 mmol) to give the title compound (6.95 mg, 17.2% yield). ¹H NMR (500 MHz, DMSO-d₆) δ=8.55 (br s, 1H), 7.90-7.81 (m, 2H), 7.73 (br d, J=7.9 Hz, 2H), 7.02 (br d, J=8.4 Hz, 2H), 6.93 (br s, 2H), 4.29-4.09 (m, 1H), 3.22-3.16 (m, 4H), 2.80-2.69 (m, 1H), 2.38 (br s, 4H), 1.99 (br s, 2H), 1.89-1.74 (m, 2H), 1.72-1.59 (m, 2H), 1.29 (br d, J=6.4 Hz, 3H); LCMS: [M+H]⁺=541.54.

### Example 151: (R)-6-(3-amino-5-fluoro-6-(3-((3-methoxypyrrolidin-1-yl)methyl)-4-morpholinophenyl)pyrazin-2-yl)-3,4-dihydroisoquinolin-1(2H)-one (I-151)

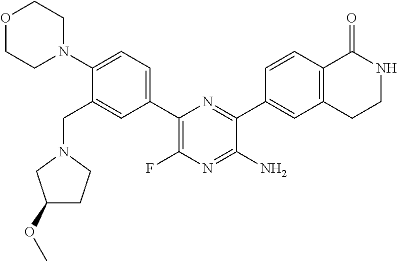 [SMILES: CO[C@@H]1CCN(Cc2cc(-c3nc(-c4ccc5c(c4)CCNC5=O)c(N)nc3F)ccc2N2CCOCC2)C1]

Prepared from 6-(3-amino-6-bromo-5-fluoropyrazin-2-yl)-3,4-dihydroisoquinolin-1(2H)-one (39.0 mg, 0.116 mmol) and (R)-4-(2-((3-methoxypyrrolidin-1-yl)methyl)-4-(4,4,5,5-tetramethyl-1,3,2-dioxaborolan-2-yl)phenyl)morpholine (65 mg, 0.150 mmol, in turn prepared from a sequence starting with 5-bromo-2-morpholinobenzaldehyde and (R)-3-Methoxy-pyrrolidine hydrochloride analogous to previous examples) to give the title compound as a beige solid (46 mg, 71% yield). ¹H NMR (500 MHz, DMSO-d₆) δ 8.0-8.0 (m, 1H), 7.95 (d, 1H, J=8.1 Hz), 7.9-7.9 (m, 1H), 7.7-7.8 (m, 1H), 7.7-7.7 (m, 1H), 7.6-7.7 (m, 1H), 7.1-7.2 (m, 1H), 6.7-6.9 (m, 2H), 3.8-3.9 (m, 1H), 3.7-3.8 (m, 4H), 3.6-3.7 (m, 2H), 3.4-3.5 (m, 2H), 3.14 (s, 3H), 2.9-3.0 (m, 6H), 2.7-2.8 (m, 1H), 2.6-2.7 (m, 1H), 2.4-2.5 (m, 2H), 1.9-2.1 (m, 1H), 1.6-1.7 (m, 1H); LCMS: [M+H]⁺=533.58.

### Example 152: (R)-6-(3-amino-5-fluoro-6-(3-((3-methoxypyrrolidin-1-yl)methyl)-4-morpholinophenyl)pyrazin-2-yl)-7-fluoro-3,4-dihydroisoquinolin-1(2H)-one (I-152)

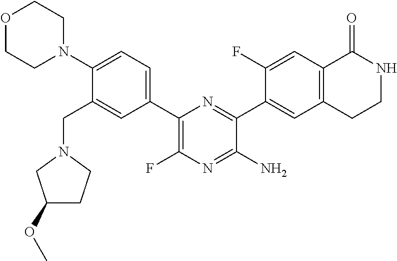 [SMILES: CO[C@@H]1CCN(Cc2cc(-c3nc(-c4cc5c(cc4F)C(=O)NCC5)c(N)nc3F)ccc2N2CCOCC2)C1]

Prepared from 6-(3-amino-6-bromo-5-fluoropyrazin-2-yl)-7-fluoro-3,4-dihydroisoquinolin-1(2H)-one (41.0 mg, 0.116 mmol) and (R)-4-(2-((3-methoxypyrrolidin-1-yl)methyl)-4-(4,4,5,5-tetramethyl-1,3,2-dioxaborolan-2-yl)phenyl)morpholine (40 mg, 0.150 mmol) to give the title compound as a beige solid (46 mg, 60% yield). ¹H NMR (500 MHz, DMSO-d₆) δ 8.1-8.2 (m, 1H), 7.8-7.9 (m, 1H), 7.7-7.7 (m, 1H), 7.65 (d, 1H, J=10.1 Hz), 7.5-7.6 (m, 1H), 7.1-7.2 (m, 1H), 6.7-6.9 (m, 2H), 3.8-3.9 (m, 1H), 3.7-3.8 (m, 4H), 3.6-3.7 (m, 2H), 3.4-3.5 (m, 2H), 3.1-3.2 (m, 3H), 2.95 (br s, 6H), 2.7-2.7 (m, 1H), 2.6-2.7 (m, 2H), 2.4-2.5 (m, 1H), 1.9-2.0 (m, 1H), 1.6-1.7 (m, 1H); LCMS: [M+H]⁺=551.56.

### Example 153: 6-(3-amino-6-(3-((dimethylamino)methyl)-4-morpholinophenyl)-5-fluoropyrazin-2-yl)-4-chloroisoquinolin-1(2H)-one (I-153)

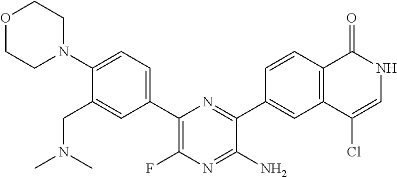 [SMILES: CN(C)Cc1cc(-c2nc(-c3ccc4c(=O)[nH]cc(Cl)c4c3)c(N)nc2F)ccc1N1CCOCC1]

### Step 1: 6-(3-amino-6-bromo-5-fluoropyrazin-2-yl)-4-chloroisoquinolin-1(2H)-one

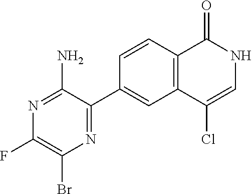 [SMILES: Nc1nc(F)c(Br)nc1-c1ccc2c(=O)[nH]cc(Cl)c2c1]

Prepared from 5-bromo-6-fluoro-3-iodopyrazin-2-amine (270 mg, 0.849 mmol) and 4-chloro-6-(4,4,5,5-tetramethyl-1,3,2-dioxaborolan-2-yl)isoquinolin-1(2H)-one (311 mg, 1.019 mmol) to give the product (123 mg, 39.2% yield) as a beige solid. ¹H NMR (500 MHz, DMSO-d₆) δ ppm 11.64 (br. s., 1H), 8.33 (d, J=8.31 Hz, 1H), 8.04 (d, J=1.22 Hz, 1H), 7.82 (dd, J=8.25, 1.53 Hz, 1H), 7.55 (br. s., 1H), 7.10 (s, 2H); LCMS: [M+H]⁺=369.24.

### Step 2: 6-(3-amino-6-(3-((dimethylamino)methyl)-4-morpholinophenyl)-5-fluoropyrazin-2-yl)-4-chloroisoquinolin-1(2H)-one

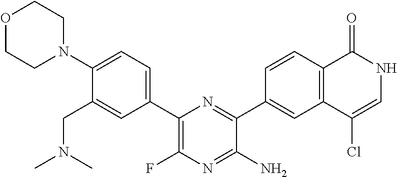 [SMILES: CN(C)Cc1cc(-c2nc(-c3ccc4c(=O)[nH]cc(Cl)c4c3)c(N)nc2F)ccc1N1CCOCC1]

Prepared from 6-(3-amino-6-bromo-5-fluoropyrazin-2-yl)-4-chloroisoquinolin-1(2H)-one (35 mg, 0.095 mmol) and N,N-dimethyl-1-(2-morpholino-5-(4,4,5,5-tetramethyl-1,3,2-dioxaborolan-2-yl)phenyl)methanamine (39.4 mg, 0.114 mmol) to give the title compound (10.1 mg, 21% yield) as a yellow solid. ¹H NMR (500 MHz, DMSO-d₆) δ ppm 11.62 (br. s., 1H), 8.35 (d, J=8.31 Hz, 1H), 8.16 (d, J=1.10 Hz, 1H), 7.95 (dd, J=8.25, 1.53 Hz, 1 H), 7.92 (s, 1H), 7.74 (d, J=8.56 Hz, 1H), 7.55 (s, 1H), 7.17 (d, J=8.56 Hz, 1H), 6.93 (s, 2H), 3.72-3.78 (m, 4H), 3.46 (s, 2H), 2.93-2.99 (m, 4H), 2.19 (s, 6H); LCMS: [M+H]⁺=509.51.

### Example 154: 6-(3-amino-6-(3-((dimethylamino)methyl)-4-(tetrahydro-2H-pyran-4-yl)phenyl)-5-fluoropyrazin-2-yl)-4-chloroisoquinolin-1(2H)-one (I-154)

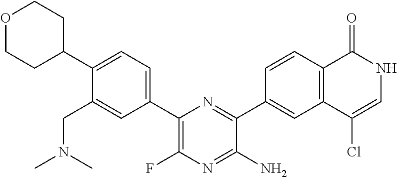 [SMILES: CN(C)Cc1cc(-c2nc(-c3ccc4c(=O)[nH]cc(Cl)c4c3)c(N)nc2F)ccc1C1CCOCC1]

Prepared from 6-(3-amino-6-bromo-5-fluoropyrazin-2-yl)-4-chloroisoquinolin-1(2H)-one (35 mg, 0.095 mmol) and N,N-dimethyl-1-(2-(tetrahydro-2H-pyran-4-yl)-5-(4,4,5,5-tetramethyl-1,3,2-dioxaborolan-2-yl)phenyl)methanamine (39.2 mg, 0.114 mmol) to give the product (16.9 mg, 35.1% yield) as a beige solid. 1H NMR (500 MHz, DMSO-d₆) b ppm 11.62 (br. s., 1H), 8.35 (d, J=8.31 Hz, 1H), 8.16 (s, 1H), 7.95 (dd, J=8.31, 1.34 Hz, 1H), 7.73-7.77 (m, 2H), 7.55 (s, 1H), 7.40 (d, J=8.80 Hz, 1H), 6.97 (s, 2H), 3.96 (dd, J=10.82, 3.24 Hz, 2H), 3.42-3.47 (m, 4H), 3.17-3.25 (m, 1H), 2.17 (s, 6H), 1.67-1.78 (m, 2H), 1.58-1.66 (m, 2H); LCMS: [M+H]⁺=508.51.

### Example 155: (R)-6-(3-amino-5-fluoro-6-(3-((3-methoxypyrrolidin-1-yl)methyl)-4-morpholinophenyl)pyrazin-2-yl)-8-fluoro-3,4-dihydroisoquinolin-1(2H)-one (I-155)

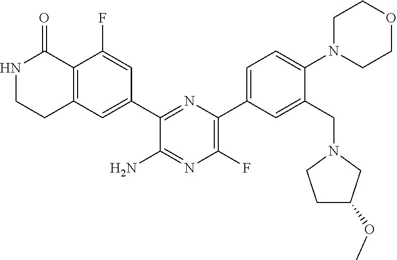 [SMILES: CO[C@@H]1CCN(Cc2cc(-c3nc(-c4cc(F)c5c(c4)CCNC5=O)c(N)nc3F)ccc2N2CCOCC2)C1]

Prepared from 6-(3-amino-6-bromo-5-fluoropyrazin-2-yl)-8-fluoro-3,4-dihydroisoquinolin-1(2H)-one (41.0 mg, 0.116 mmol) and (R)-4-(2-((3-methoxypyrrolidin-1-yl)methyl)-4-(4,4,5,5-tetramethyl-1,3,2-dioxaborolan-2-yl)phenyl)morpholine (65 mg, 0.150 mmol) to give the title compound as a pale-yellow solid (41 mg, 61% yield). ¹H NMR (500 MHz, DMSO-d₆) δ 8.0-8.1 (m, 1H), 7.9-7.9 (m, 1H), 7.7-7.8 (m, 1H), 7.5-7.5 (m, 1H), 7.4-7.5 (m, 1H), 7.1-7.2 (m, 1H), 6.9-7.0 (m, 2H), 3.9-3.9 (m, 1H), 3.7-3.8 (m, 4H), 3.6-3.7 (m, 2H), 3.38 (br d, 2H, J=3.5 Hz), 3.1-3.2 (m, 3H), 2.9-3.0 (m, 6H), 2.7-2.8 (m, 1H), 2.6-2.7 (m, 2H), 2.55 (br d, 1H, J=1.6 Hz), 1.9-2.0 (m, 1H), 1.6-1.7 (m, 1H); LCMS: [M+H]⁺=551.56.

### Example 156: (S)-6-(3-amino-5-fluoro-6-(3-((3-methoxypyrrolidin-1-yl)methyl)-4-morpholinophenyl)pyrazin-2-yl)-3,4-dihydroisoquinolin-1(2H)-one (I-156)

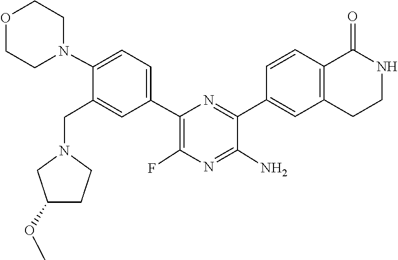 [SMILES: CO[C@H]1CCN(Cc2cc(-c3nc(-c4ccc5c(c4)CCNC5=O)c(N)nc3F)ccc2N2CCOCC2)C1]

Prepared from 6-(3-amino-6-bromo-5-fluoropyrazin-2-yl)-3,4-dihydroisoquinolin-1(2H)-one (37.7 mg, 0.112 mmol) and (S)-4-(2-((3-methoxypyrrolidin-1-yl)methyl)-4-(4,4,5,5-tetramethyl-1,3,2-dioxaborolan-2-yl)phenyl)morpholine (65 mg, 0.145 mmol) to give the title compound as a beige solid (37 mg, 59% yield). ¹H NMR (500 MHz, DMSO-d₆) δ 8.0-8.0 (m, 1H), 7.9-8.0 (m, 1H), 7.9-7.9 (m, 1H), 7.7-7.8 (m, 1H), 7.7-7.7 (m, 1H), 7.65 (s, 1H), 7.1-7.2 (m, 1H), 6.7-6.9 (m, 2H), 3.9-3.9 (m, 1H), 3.7-3.8 (m, 4H), 3.6-3.7 (m, 2H), 3.4-3.5 (m, 2H), 3.1-3.2 (m, 3H), 3.0-3.0 (m, 2H), 2.95 (br s, 4H), 2.7-2.8 (m, 1H), 2.6-2.7 (m, 2H), 2.4-2.5 (m, 1H), 1.9-2.0 (m, 1H), 1.6-1.7 (m, 1H); LCMS: [M+H]⁺=533.64.

### Example 157: (S)-6-(3-amino-5-fluoro-6-(3-((3-methoxypyrrolidin-1-yl)methyl)-4-morpholinophenyl)pyrazin-2-yl)-7-fluoro-3,4-dihydroisoquinolin-1(2H)-one (I-157)

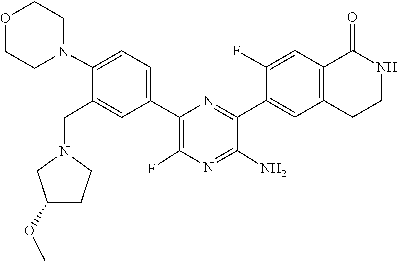 [SMILES: CO[C@H]1CCN(Cc2cc(-c3nc(-c4cc5c(cc4F)C(=O)NCC5)c(N)nc3F)ccc2N2CCOCC2)C1]

Prepared from 6-(3-amino-6-bromo-5-fluoropyrazin-2-yl)-7-fluoro-3,4-dihydroisoquinolin-1(2H)-one (39.7 mg, 0.112 mmol) and (S)-4-(2-((3-methoxypyrrolidin-1-yl)methyl)-4-(4,4,5,5-tetramethyl-1,3,2-dioxaborolan-2-yl)phenyl)morpholine (65 mg, 0.145 mmol) to give the title compound as a beige solid (43 mg, 66% yield). ¹H NMR (500 MHz, DMSO-d₆) δ 8.1-8.2 (m, 1H), 7.8-7.9 (m, 1H), 7.7-7.7 (m, 1H), 7.6-7.7 (m, 1H), 7.5-7.5 (m, 1H), 7.1-7.2 (m, 1H), 6.7-6.9 (m, 2H), 3.8-3.9 (m, 1H), 3.7-3.8 (m, 4H), 3.6-3.7 (m, 2H), 3.4-3.5 (m, 2H), 3.1-3.2 (m, 3H), 2.9-3.0 (m, 6H), 2.7-2.7 (m, 1H), 2.6-2.7 (m, 2H), 2.4-2.5 (m, 1H), 1.9-2.0 (m, 1H), 1.6-1.7 (m, 1H); LCMS: [M+H]⁺=551.69.

### Example 158: (S)-6-(3-amino-5-fluoro-6-(3-((3-methoxypyrrolidin-1-yl)methyl)-4-morpholinophenyl)pyrazin-2-yl)-8-fluoro-3,4-dihydroisoquinolin-1(2H)-one (I-158)

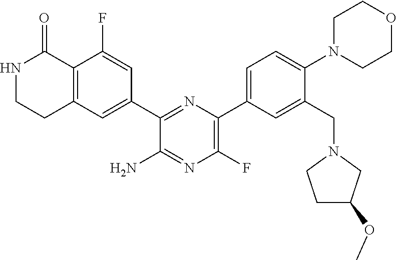 [SMILES: CO[C@H]1CCN(Cc2cc(-c3nc(-c4cc(F)c5c(c4)CCNC5=O)c(N)nc3F)ccc2N2CCOCC2)C1]

Prepared from 6-(3-amino-6-bromo-5-fluoropyrazin-2-yl)-8-fluoro-3,4-dihydroisoquinolin-1(2H)-one (39.7 mg, 0.112 mmol) and (S)-4-(2-((3-methoxypyrrolidin-1-yl)methyl)-4-(4,4,5,5-tetramethyl-1,3,2-dioxaborolan-2-yl)phenyl)morpholine (65 mg, 0.145 mmol) to give the title compound as a pale-yellow solid (46 mg, 71% yield). ¹H NMR (500 MHz, DMSO-d₆) δ=8.09-8.02 (m, 1H), 7.93-7.87 (m, 1H), 7.77-7.70 (m, 1H), 7.54-7.49 (m, 1H), 7.47-7.41 (m, 1H), 7.21-7.14 (m, 1H), 7.00-6.90 (m, 2H), 3.91-3.87 (m, 1H), 3.75 (br d, J=4.0 Hz, 4H), 3.70-3.65 (m, 2H), 3.39-3.37 (m, 2H), 3.14 (s, 3H), 3.00-2.94 (m, 6H), 2.79-2.69 (m, 2H), 2.66-2.61 (m, 2H), 2.03-1.95 (m, 1H), 1.72-1.61 (m, 1H); LCMS: [M+H]⁺=551.50.

### Example 159: 6-(3-amino-6-(4-(4-(4,4-difluorobutyl)piperazin-1-yl)phenyl)-5-fluoropyrazin-2-yl)-8-fluoro-3-methylisoquinolin-1(2H)-one (I-159)

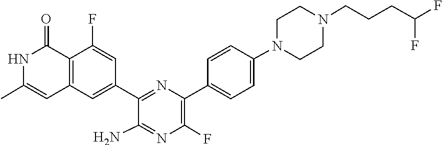 [SMILES: Cc1cc2cc(-c3nc(-c4ccc(N5CCN(CCCC(F)F)CC5)cc4)c(F)nc3N)cc(F)c2c(=O)[nH]1]

### Step 1: 6-(3-amino-5-fluoro-6-(4-(piperazin-1-yl)phenyl)pyrazin-2-yl)-8-fluoro-3-methylisoquinolin-1(2H)-one

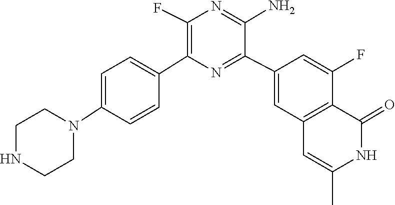 [SMILES: Cc1cc2cc(-c3nc(-c4ccc(N5CCNCC5)cc4)c(F)nc3N)cc(F)c2c(=O)[nH]1]

Prepared from 6-(3-amino-6-bromo-5-fluoropyrazin-2-yl)-8-fluoro-3-methylisoquinolin-1(2H)-one (22 mg, 0.060 mmol) and 4-piperazinylphenylboronic acid, pinacol ester (22.45 mg, 0.078 mmol) to give the product as a yellow solid. (21 mg, 67% yield); LCMS: [M+H]⁺=449.47.

### Step 2: 6-(3-amino-6-(4-(4-(4,4-difluorobutyl)piperazin-1-yl)phenyl)-5-fluoropyrazin-2-yl)-8-fluoro-3-methylisoquinolin-1(2H)-one

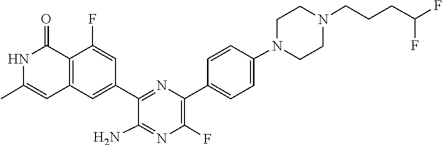 [SMILES: Cc1cc2cc(-c3nc(-c4ccc(N5CCN(CCCC(F)F)CC5)cc4)c(F)nc3N)cc(F)c2c(=O)[nH]1]

Prepared from 6-(3-amino-5-fluoro-6-(4-(piperazin-1-yl)phenyl)pyrazin-2-yl)-8-fluoro-3-methylisoquinolin-1(2H)-one (21 mg, 0.047 mmol) and 4-bromo-1,1-difluorobutane (16.20 mg, 0.094 mmol) to give the title product as a yellow solid. (10 mg, 37.5% yield); ¹H NMR (500 MHz, DMSO-d₆) δ=11.39-11.26 (m, 1H), 7.83-7.73 (m, 2H), 7.71-7.67 (m, 1H), 7.43-7.37 (m, 1H), 7.09-6.99 (m, 2H), 6.94-6.72 (m, 2H), 6.46-6.40 (m, 1H), 6.26-5.99 (m, 1H), 3.21 (br s, 4H), 2.60-2.54 (m, 4H), 2.38 (br d, J=5.7 Hz, 2H), 2.26-2.20 (m, 3H), 1.93-1.80 (m, 2H), 1.64-1.54 (m, 2H); LCMS: [M+H]⁺=541.60.

### Example 160: 6-(3-amino-5-fluoro-6-(3-(((2-methoxyethyl)(methyl)amino)methyl)-4-morpholinophenyl)pyrazin-2-yl)-3,4-dihydroisoquinolin-1(2H)-one (I-161)

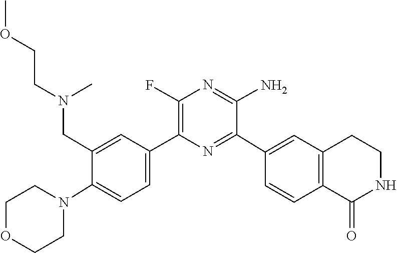 [SMILES: COCCN(C)Cc1cc(-c2nc(-c3ccc4c(c3)CCNC4=O)c(N)nc2F)ccc1N1CCOCC1]

Prepared from 6-(3-amino-6-bromo-5-fluoropyrazin-2-yl)-3,4-dihydroisoquinolin-1(2H)-one (44.2 mg, 0.131 mmol) and 2-methoxy-N-methyl-N-(2-morpholino-5-(4,4,5,5-tetramethyl-1,3,2-dioxaborolan-2-yl)benzyl)ethan-1-amine (70 mg, 0.170 mmol, in turn prepared from a sequence starting with 5-bromo-2-morpholinobenzaldehyde and N-(2-methoxyethyl)methylamine using methods analogous to previous examples) to give the title compound as a beige solid (41 mg, 57% yield). ¹H NMR (500 MHz, DMSO-d₆) δ=8.01-7.97 (m, 1H), 7.97-7.92 (m, 2H), 7.76-7.71 (m, 1H), 7.71-7.67 (m, 1H), 7.66-7.61 (m, 1H), 7.21-7.15 (m, 1H), 6.87-6.77 (m, 2H), 3.78-3.74 (m, 4H), 3.62-3.58 (m, 2H), 3.47-3.43 (m, 4H), 3.22-3.18 (m, 3H), 3.02-2.98 (m, 2H), 2.97-2.92 (m, 4H), 2.58-2.56 (m, 2H), 2.25-2.17 (m, 3H); LCMS: [M+H]⁺=521.61.

### Example 161: 6-(3-amino-5-fluoro-6-(3-(((2-methoxyethyl)(methyl)amino)methyl)-4-morpholinophenyl)pyrazin-2-yl)-7-fluoro-3,4-dihydroisoquinolin-1(2H)-one (I-119)

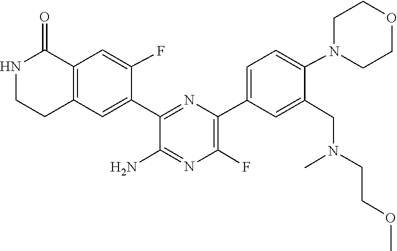 [SMILES: COCCN(C)Cc1cc(-c2nc(-c3cc4c(cc3F)C(=O)NCC4)c(N)nc2F)ccc1N1CCOCC1]

Prepared from 6-(3-amino-6-bromo-5-fluoropyrazin-2-yl)-7-fluoro-3,4-dihydroisoquinolin-1(2H)-one (49.0 mg, 0.138 mmol) and 2-methoxy-N-methyl-N-(2-morpholino-5-(4,4,5,5-tetramethyl-1,3,2-dioxaborolan-2-yl)benzyl)ethan-1-amine (70 mg, 0.179 mmol) to give the title compound as a beige solid (39 mg, 50% yield). ¹H NMR (500 MHz, DMSO-d₆) δ=8.20-8.13 (m, 1H), 7.93-7.88 (m, 1H), 7.74-7.66 (m, 1H), 7.66-7.62 (m, 1H), 7.53-7.50 (m, 1H), 7.22-7.09 (m, 1H), 6.86-6.68 (m, 2H), 3.80-3.73 (m, 4H), 3.63-3.55 (m, 2H), 3.46-3.41 (m, 4H), 3.19 (br s, 3H), 2.99-2.91 (m, 6H), 2.56-2.53 (m, 2H), 2.32-2.07 (m, 3H); LCMS: [M+H]⁺=539.59.

### Example 162: 6-(3-amino-5-fluoro-6-(3-(((2-methoxyethyl)(methyl)amino)methyl)-4-morpholinophenyl)pyrazin-2-yl)-8-fluoro-3,4-dihydroisoquinolin-1(2H)-one (I-162)

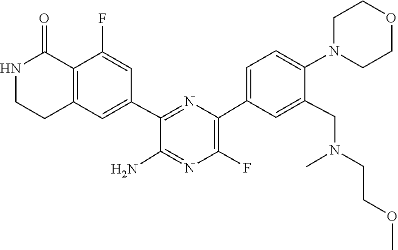 [SMILES: COCCN(C)Cc1cc(-c2nc(-c3cc(F)c4c(c3)CCNC4=O)c(N)nc2F)ccc1N1CCOCC1]

Prepared from 6-(3-amino-6-bromo-5-fluoropyrazin-2-yl)-8-fluoro-3,4-dihydroisoquinolin-1(2H)-one (49.0 mg, 0.138 mmol) and 2-methoxy-N-methyl-N-(2-morpholino-5-(4,4,5,5-tetramethyl-1,3,2-dioxaborolan-2-yl)benzyl)ethan-1-amine (70 mg, 0.179 mmol) to give the title compound as a yellow solid (45 mg, 57.5% yield). ¹H NMR (500 MHz, DMSO-d₆) δ=8.09-8.02 (m, 1H), 7.97-7.92 (m, 1H), 7.77-7.71 (m, 1H), 7.53-7.49 (m, 1H), 7.46-7.41 (m, 1H), 7.21-7.15 (m, 1H), 6.99-6.90 (m, 2H), 3.79-3.74 (m, 4H), 3.62-3.57 (m, 2H), 3.48-3.44 (m, 2H), 3.37 (br d, J=5.1 Hz, 2H), 3.22-3.16 (m, 3H), 3.01-2.96 (m, 2H), 2.96-2.91 (m, 4H), 2.58-2.55 (m, 2H), 2.28-2.19 (m, 3H); LCMS: [M+H]⁺=539.53.

### Example 163: 6-(3-amino-6-(3-((ethyl(methyl)amino)methyl)-4-(tetrahydro-2H-pyran-4-yl)phenyl)-5-fluoropyrazin-2-yl)-3,4-dihydroisoquinolin-1(2H)-one (I-163)

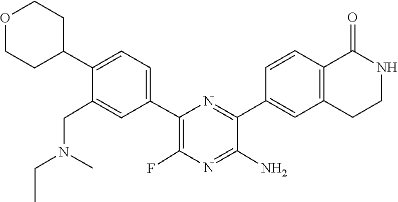 [SMILES: CCN(C)Cc1cc(-c2nc(-c3ccc4c(c3)CCNC4=O)c(N)nc2F)ccc1C1CCOCC1]

### Step 1: N-(5-bromo-2-(tetrahydro-2H-pyran-4-yl)benzyl)-N-methylethanamine

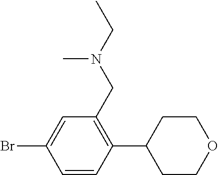 [SMILES: CCN(C)Cc1cc(Br)ccc1C1CCOCC1]

Prepared by reacting 5-bromo-2-(tetrahydro-2H-pyran-4-yl)benzaldehyde (400 mg, 1.486 mmol) with N-ethylmethylamine (0.511 ml, 5.94 mmol) in a manner similar to previous procedures. The title compound was isolated as a white solid (444 mg, 91% yield) LCMS: [M+H]⁺=312.38

### Step 2: N-methyl-N-(2-(tetrahydro-2H-pyran-4-yl)-5-(4,4,5,5-tetramethyl-1,3,2-dioxaborolan-2-yl)benzyl)ethanamine

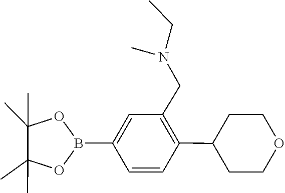 [SMILES: CCN(C)Cc1cc(B2OC(C)(C)C(C)(C)O2)ccc1C1CCOCC1]

Prepared from N-(5-bromo-2-(tetrahydro-2H-pyran-4-yl)benzyl)-N-methylethanamine (190 mg, 0.578 mmol) to give the product as a brown solid (208 mg, 90% yield based on 90% purity) LCMS: [M+H]⁺=360.34

### Step 3: 6-(3-amino-6-(3-((ethyl(methyl)amino)methyl)-4-(tetrahydro-2H-pyran-4-yl)phenyl)-5-fluoropyrazin-2-yl)-3,4-dihydroisoquinolin-1(2H)-one

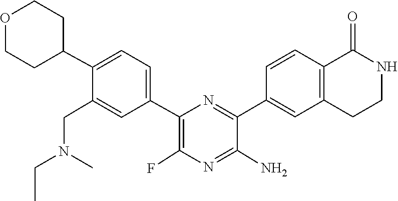 [SMILES: CCN(C)Cc1cc(-c2nc(-c3ccc4c(c3)CCNC4=O)c(N)nc2F)ccc1C1CCOCC1]

Prepared from 6-(3-amino-6-bromo-5-fluoropyrazin-2-yl)-3,4-dihydroisoquinolin-1(2H)-one (42.2 mg, 0.125 mmol) and N-methyl-N-(2-(tetrahydro-2H-pyran-4-yl)-5-(4,4,5,5-tetramethyl-1,3,2-dioxaborolan-2-yl)benzyl)ethanamine (65 mg, 0.163 mmol) to give the title compound as a beige solid (43, 67% yield). ¹H NMR (500 MHz, DMSO-d₆) δ=8.03-7.98 (m, 1H), 7.98-7.94 (m, 1H), 7.78-7.72 (m, 2H), 7.71-7.68 (m, 1H), 7.67-7.63 (m, 1H), 7.43-7.37 (m, 1H), 6.95-6.79 (m, 2H), 4.01-3.96 (m, 2H), 3.56-3.51 (m, 2H), 3.47-3.42 (m, 4H), 3.28-3.21 (m, 1H), 3.02-2.97 (m, 2H), 2.44-2.37 (m, 2H), 2.16-2.08 (m, 3H), 1.76-1.68 (m, 2H), 1.66-1.61 (m, 2H), 1.09-1.04 (m, 3H); LCMS: [M+H]⁺=490.52.

### Example 164: 6-(3-amino-6-(3-((ethyl(methyl)amino)methyl)-4-(tetrahydro-2H-pyran-4-yl)phenyl)-5-fluoropyrazin-2-yl)-7-fluoro-3,4-dihydroisoquinolin-1(2H)-one (I-164)

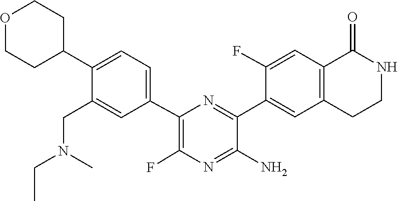 [SMILES: CCN(C)Cc1cc(-c2nc(-c3cc4c(cc3F)C(=O)NCC4)c(N)nc2F)ccc1C1CCOCC1]

Prepared from 6-(3-amino-6-bromo-5-fluoropyrazin-2-yl)-7-fluoro-3,4-dihydroisoquinolin-1(2H)-one (44.5 mg, 0.125 mmol) and N-methyl-N-(2-(tetrahydro-2H-pyran-4-yl)-5-(4,4,5,5-tetramethyl-1,3,2-dioxaborolan-2-yl)benzyl)ethanamine (65 mg, 0.163 mmol) to give the title compound as a beige solid (33, 49% yield). 1H NMR (500 MHz, DMSO-d6) δ=8.22-8.09 (m, 1H), 7.76-7.62 (m, 3H), 7.56-7.49 (m, 1H), 7.42-7.35 (m, 1H), 6.91-6.74 (m, 2H), 4.01-3.93 (m, 2H), 3.55-3.50 (m, 2H), 3.47-3.41 (m, 4H), 3.27-3.20 (m, 1H), 2.99-2.92 (m, 2H), 2.42-2.34 (m, 2H), 2.14-2.05 (m, 3H), 1.78-1.68 (m, 2H), 1.66-1.59 (m, 2H), 1.12-1.01 (m, 3H); LCMS: [M+H]⁺=508.63

### Example 165: 6-(3-amino-6-(3-((ethyl(methyl)amino)methyl)-4-(tetrahydro-2H-pyran-4-yl)phenyl)-5-fluoropyrazin-2-yl)-8-fluoro-3,4-dihydroisoquinolin-1(2H)-one (I-165)

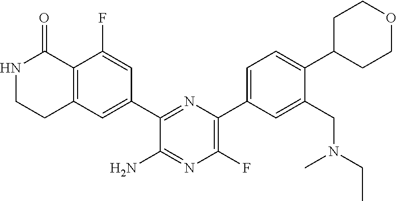 [SMILES: CCN(C)Cc1cc(-c2nc(-c3cc(F)c4c(c3)CCNC4=O)c(N)nc2F)ccc1C1CCOCC1]

Prepared from N-methyl-N-(2-(tetrahydro-2H-pyran-4-yl)-5-(4,4,5,5-tetramethyl-1,3,2-dioxaborolan-2-yl)benzyl)ethanamine (65 mg, 0.163 mmol) and N-methyl-N-(2-(tetrahydro-2H-pyran-4-yl)-5-(4,4,5,5-tetramethyl-1,3,2-dioxaborolan-2-yl)benzyl)ethanamine (65 mg, 0.163 mmol) to give the title compound as a pale-yellow solid (46, 69% yield). ¹H NMR (500 MHz, DMSO-d₆) δ=8.09-8.02 (m, 1H), 7.78-7.71 (m, 2H), 7.52-7.49 (m, 1H), 7.46-7.38 (m, 2H), 7.03-6.90 (m, 2H), 4.02-3.95 (m, 2H), 3.55-3.51 (m, 2H), 3.47-3.39 (m, 4H), 3.28-3.22 (m, 1H), 3.00-2.95 (m, 2H), 2.44-2.38 (m, 2H), 2.16-2.09 (m, 3H), 1.77-1.69 (m, 2H), 1.67-1.61 (m, 2H), 1.08-1.04 (m, 3H); LCMS: [M+H]⁺=508.57.

### Example 166: 6-(3-amino-5-fluoro-6-(4-(4-isopropylpiperazin-1-yl)phenyl)pyrazin-2-yl)-8-fluoro-4-methylisoquinolin-1(2H)-one (I-166)

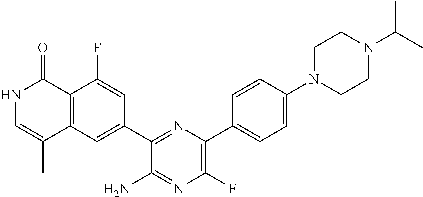 [SMILES: Cc1c[nH]c(=O)c2c(F)cc(-c3nc(-c4ccc(N5CCN(C(C)C)CC5)cc4)c(F)nc3N)cc12]

### Step 1: (E)-3-(3-bromo-5-fluorophenyl)but-2-enoic acid

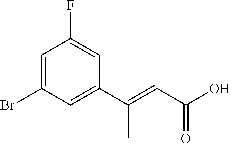

To THF (75 mL) at 0° C. under nitrogen atmosphere, sodium hydride (60%, 1.38 g, 34.56 mmol) was add portion wise. Triethyl phosphonoacetate (6.85 mL, 34.56 mmol) was added dropwise and stirred at 0° C. for 20 min. The resulting reaction mass was added dropwise to a solution of 1-(3-bromo-5-fluorophenyl)ethan-1-one (5 g, 23.04 mmol) in THF (50 mL) and the mixture was refluxed for 16 h. The mixture was then diluted with water (200 mL) and extracted with DCM (3×150 mL). The organic layer was dried over Na₂SO₄ and concentrated under vacuum to afford a yellow oil which was dissolved in MeOH (25 mL) and NaOH (4.6 g, 226.13 mmol) and water (15 mL) were added and the reaction was heated at 50° C. for 2 h. The mixture was concentrated and the resulting aqueous solution was acidified by aqueous 2M HCl and extracted with EtOAc (2×150 mL). The combined organic layers were dried over anhydrous Na₂SO₄ and concentrated under vacuum to afford the product (3.5 g, 58.6% yield) as white solid. LCMS: [M+H]⁺=258.9.

### Step 2: (E)-3-(3-bromo-5-fluorophenyl)but-2-enoyl chloride

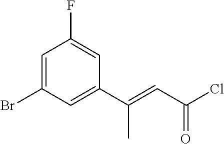

To a cooled solution of (E)-3-(3-bromo-5-fluorophenyl)but-2-enoic acid (3.5 g, 13.58 mmol) in DCM (35 mL) and DMF (0.2 mL) at 0° C., oxalyl chloride (2.05 g, 16.21 mmol) was added dropwise and the reaction was allowed to warm to RT and stirred for 3 h. The mixture was concentrated and azeotroped with toluene (2×20 mL) and DCM (2×20 mL) to give the crude product (3.5 g,) as white solid which was used directly in the next step without analysis.

### Step 3: (E)-3-(3-bromo-5-fluorophenyl)but-2-enoyl azide

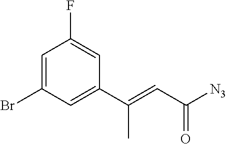 [SMILES: C/C(=C\C(=O)N=[N+]=[N-])c1cc(F)cc(Br)c1]

To a cooled solution of the (E)-3-(3-bromo-5-fluorophenyl)but-2-enoyl chloride (3.5 g, 12.61 mmol) in 1,4 dioxane (35 mL) at 0° C., a suspension of sodium azide (1.47 g, 22.7 mmol) in 1:1 mixture of 1,4-dioxane and water (15 mL) was added and the mixture was gradually warmed to room temperature and stirred for 1.5 h. The mixture was then diluted with water (30 mL) and extracted with diethyl ether (2×100 mL). The combined organic layer was back washed with saturated aqueous NaHCO₃ solution (3×100 mL) and water (3×100 mL) and dried over Na₂SO₄. The organic layer was directly used for next step.

### Step 4: 6-bromo-8-fluoro-4-methylisoquinolin-1(2H)-one

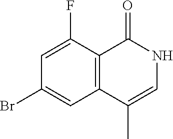

To the ether layer of (E)-3-(3-bromo-5-fluorophenyl)but-2-enoyl azide was treated with 1,2 dichlorobenzene (30 mL) and the ether was removed under vacuum to give a solution of (E)-3-(3-bromo-5-fluorophenyl)but-2-enoyl azide in 1,2 dichlorobenzene. The acyl azide solution in 1,2 dichlorobenzene was added dropwise over 30 min to a solution of iodine (0.4 g) in 1,2 dichlorobenzene (30 mL) at 120° C. The mixture was then stirred at 190° C. for 16 h, and allowed to cool room temperature and added to hexane (1000 mL). The suspension was stirred for 1 h and the resulting solid was filtered, washed with EtOAc (50 mL) and DCM (50 mL) and dried under vacuum to give the title compound (0.4 g, 12.7% yield) as pale yellow solid. LCMS: [M+H]⁺=258.

### Step 5: (8-fluoro-4-methyl-1-oxo-1,2-dihydroisoquinolin-6-yl)boronic acid

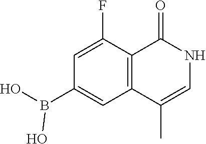 [SMILES: Cc1c[nH]c(=O)c2c(F)cc(B(O)O)cc12]

Prepared from 6-bromo-8-fluoro-4-methylisoquinolin-1(2H)-one (400 mg, 1.562 mmol) to give a mixture of the boronic acid and boronate which was used in the next step without further purification. LCMS: [M+H]+ 222.29 boronic acid, 304.42 boronate.

### Step 6: 6-(3-amino-6-bromo-5-fluoropyrazin-2-yl)-8-fluoro-4-methylisoquinolin-1(2H)-one

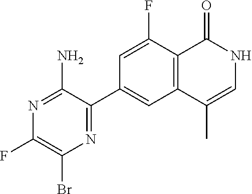 [SMILES: Cc1c[nH]c(=O)c2c(F)cc(-c3nc(Br)c(F)nc3N)cc12]

Prepared from 5-bromo-6-fluoro-3-iodopyrazin-2-amine (200 mg, 0.629 mmol) and (8-fluoro-4-methyl-1-oxo-1,2-dihydroisoquinolin-6-yl)boronic acid (167 mg, 0.755 mmol) to give the product (120 mg, 51.9% yield) as a beige solid. LCMS: [M+H]⁺=367.30.

### Step 7: 6-(3-amino-5-fluoro-6-(4-(4-isopropylpiperazin-1-yl)phenyl)pyrazin-2-yl)-8-fluoro-4-methylisoquinolin-1(2H)-one

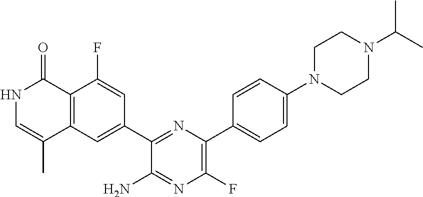 [SMILES: Cc1c[nH]c(=O)c2c(F)cc(-c3nc(-c4ccc(N5CCN(C(C)C)CC5)cc4)c(F)nc3N)cc12]

Prepared from 6-(3-amino-6-bromo-5-fluoropyrazin-2-yl)-8-fluoro-4-methylisoquinolin-1(2H)-one (30 mg, 0.082 mmol) and 4-(4-isopropylpiperazinyl)phenylboronic acid, pinacol ester (32.4 mg, 0.098 mmol) to give the product (20.1 mg, 50.1% yield) as a yellow solid. ¹H NMR (500 MHz, DMSO-d₆) δ ppm 11.14 (br. s., 1H), 7.73-7.81 (m, 3H), 7.52 (d, J=12.35 Hz, 1H), 7.09 (br. s., 1H), 7.02 (d, J=8.80 Hz, 2H), 6.89 (s, 2H), 3.18 (d, J=4.52 Hz, 4H), 2.65-2.71 (m, 1H), 2.56-2.60 (m, 4H), 2.23 (s, 3H), 1.01 (d, J=6.48 Hz, 6H); LCMS: [M+H]⁺=491.52.

### Example 167: 6-(3-amino-6-(3-((dimethylamino)methyl)-4-(tetrahydro-2H-pyran-4-yl)phenyl)-5-fluoropyrazin-2-yl)-8-fluoro-4-methylisoquinolin-1(2H)-one (I-167)

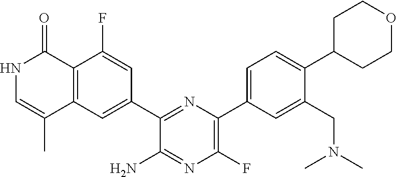 [SMILES: Cc1c[nH]c(=O)c2c(F)cc(-c3nc(-c4ccc(C5CCOCC5)c(CN(C)C)c4)c(F)nc3N)cc12]

Prepared from 6-(3-amino-6-bromo-5-fluoropyrazin-2-yl)-8-fluoro-4-methylisoquinolin-1(2H)-one (30 mg, 0.082 mmol) and N,N-dimethyl-1-(2-(tetrahydro-2H-pyran-4-yl)-5-(4,4,5,5-tetramethyl-1,3,2-dioxaborolan-2-yl)phenyl)methanamine (33.9 mg, 0.098 mmol) to give the title compound (15.7 mg, 38.0% yield) as a yellow solid. ¹H NMR (500 MHz, DMSO-d₆) δ ppm 11.14 (d, J=3.55 Hz, 1H), 7.70-7.83 (m, 3H), 7.52 (d, J=12.47 Hz, 1H), 7.40 (d, J=7.95 Hz, 1H), 7.10 (d, J=4.52 Hz, 1H), 7.04 (br. s., 2H), 3.96 (d, J=8.19 Hz, 2H), 3.41-3.50 (m, 4H), 3.21 (t, J=11.13 Hz, 1H), 2.23 (s, 3H), 2.17 (s, 6H), 1.67-1.78 (m, 2H), 1.58-1.66 (m, 2H); LCMS: [M+H]⁺=506.50.

### Example 168: 6-(3-amino-5-fluoro-6-(4-(4-isopropylpiperazin-1-yl)phenyl)pyrazin-2-yl)-4,7-difluoro-3-methylisoquinolin-1(2H)-one (I-168)

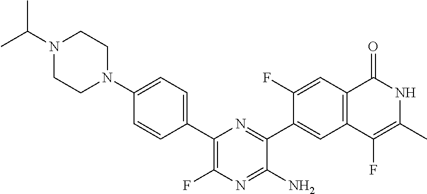 [SMILES: Cc1[nH]c(=O)c2cc(F)c(-c3nc(-c4ccc(N5CCN(C(C)C)CC5)cc4)c(F)nc3N)cc2c1F]

### Step 1: 6-bromo-7-fluoro-3-methylisoquinolin-1(2H)-one

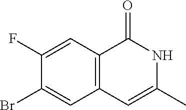

To a RBF was added copper(I) bromide (0.068 g, 0.472 mmol), Cs₂CO₃ (3.07 g, 9.43 mmol), 2,4-dibromo-5-fluorobenzamide (1.40 g, 4.72 mmol), propan-2-one (1.731 ml, 23.58 mmol) and dimethylsulfoxide (20 ml). The reaction was stirred and heated at 80° C. overnight. The mixture was partitioned between brine (200 ml) and DCM (200 ml). The organic layer was separated, and the aqueous layer washed with DCM (2×100 ml). The combined organic layers were washed with brine, dried over anhydrous Na₂SO₄, and concentrated onto celite. The mixture was purified by flash sgc eluting with 0-10% MeOH/DCM+1% NH₄OH. The desired fractions were collected, concentrated and dried under vacuum to afford the product (489 mg, 40.5% yield) as a beige solid. LCMS: [M+H]⁺=256.21.

### Step 2: 6-bromo-4,7-difluoro-3-methylisoquinolin-1(2H)-one

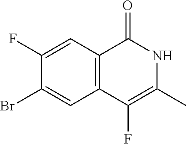

A vial was charged with 6-bromo-7-fluoro-3-methylisoquinolin-1(2H)-one (189 mg, 0.738 mmol) and Selectfluor™ fluorinating reagent >95% in F+ active (275 mg, 0.775 mmol). Methanol (10 ml) and acetonitrile (10 ml) were added, and the reaction was stirred at room temperature for 5 days. The white suspension was filtered, washed with water, and dried under vacuum to afford the product (158 mg, 78% yield) as a white solid. The material was used in the next step without further purification. LCMS [M+H]⁺=274.27.

### Step 3: (4,7-difluoro-3-methyl-1-oxo-1,2-dihydroisoquinolin-6-yl)boronic acid

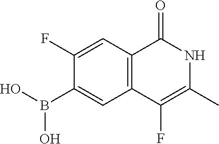 [SMILES: Cc1[nH]c(=O)c2cc(F)c(B(O)O)cc2c1F]

Prepared from 6-bromo-4,7-difluoro-3-methylisoquinolin-1(2H)-one (115 mg, 0.420 mmol) to give the boronic acid which was used in the next step without further purification. LCMS: [M+H]+=240.28.

### Step 4: 6-(3-amino-5-fluoro-6-(4-(4-isopropylpiperazin-1-yl)phenyl)pyrazin-2-yl)-4,7-difluoro-3-methylisoquinolin-1(2H)-one

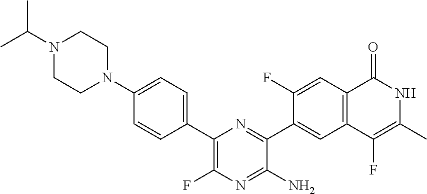 [SMILES: Cc1[nH]c(=O)c2cc(F)c(-c3nc(-c4ccc(N5CCN(C(C)C)CC5)cc4)c(F)nc3N)cc2c1F]

Prepared from (4,7-difluoro-3-methyl-1-oxo-1,2-dihydroisoquinolin-6-yl)boronic acid (32.7 mg, 0.137 mmol) and 3-bromo-6-fluoro-5-(4-(4-isopropylpiperazin-1-yl)phenyl)pyrazin-2-amine (45 mg, 0.114 mmol, prepared) to give the title compound (26.3 mg, 45.3% yield) as a yellow solid. ¹H NMR (500 MHz, DMSO-d₆) δ ppm 11.42 (br. s., 1H), 7.95 (d, J=9.90 Hz, 1H), 7.83 (d, J=6.48 Hz, 1H), 7.69 (d, J=8.07 Hz, 2H), 6.99 (d, J=8.93 Hz, 2H), 6.75 (s, 2H), 3.14-3.21 (m, 4H), 2.66 (dt, J=13.02, 6.57 Hz, 1H), 2.54-2.59 (m, 4H), 2.24 (d, J=2.69 Hz, 3H), 1.00 (d, J=6.48 Hz, 6H); LCMS: [M+H]⁺=509.45.

### Example 169: 6-(3-amino-6-(3-(azetidin-1-ylmethyl)-4-(tetrahydro-2H-pyran-4-yl)phenyl)-5-fluoropyrazin-2-yl)-3,4-dihydroisoquinolin-1(2H)-one (I-169)

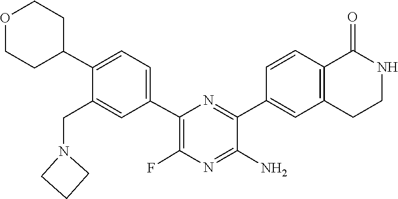 [SMILES: Nc1nc(F)c(-c2ccc(C3CCOCC3)c(CN3CCC3)c2)nc1-c1ccc2c(c1)CCNC2=O]

Prepared from 6-(3-amino-6-bromo-5-fluoropyrazin-2-yl)-3,4-dihydroisoquinolin-1(2H)-one (44.8 mg, 0.133 mmol) and 1-(2-(tetrahydro-2H-pyran-4-yl)-5-(4,4,5,5-tetramethyl-1,3,2-dioxaborolan-2-yl)benzyl)azetidine (65 mg, 0.173 mmol, prepared from a sequence starting from 5-bromo-2-(tetrahydro-2H-pyran-4-yl)benzaldehyde and azetidine using procedures analogous to those in earlier examples) to give the title compound as a beige solid (37 mg, 54% yield). ¹H NMR (DMSO-d₆, 500 MHz) δ 8.0-8.1 (m, 1H), 7.9-8.0 (m, 1H), 7.8-7.8 (m, 1H), 7.7-7.7 (m, 2H), 7.6-7.7 (m, 1H), 7.3-7.4 (m, 1H), 6.8-6.9 (m, 2H), 3.9-4.0 (m, 2H), 3.6-3.7 (m, 2H), 3.5-3.5 (m, 2H), 3.4-3.5 (m, 2H), 3.1-3.2 (m, 4H), 3.1-3.1 (m, 1H), 2.99 (brt, 2H, J=6.2 Hz), 2.0-2.0 (m, 2H), 1.7-1.8 (m, 2H), 1.6-1.7 (m, 2H); LCMS: [M+H]⁺=488.52.

### Example 170: 6-(3-amino-6-(3-(azetidin-1-ylmethyl)-4-(tetrahydro-2H-pyran-4-yl)phenyl)-5-fluoropyrazin-2-yl)-7-fluoro-3,4-dihydroisoquinolin-1(2H)-one (I-170)

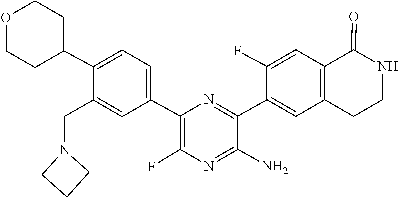 [SMILES: Nc1nc(F)c(-c2ccc(C3CCOCC3)c(CN3CCC3)c2)nc1-c1cc2c(cc1F)C(=O)NCC2]

Prepared from 6-(3-amino-6-bromo-5-fluoropyrazin-2-yl)-7-fluoro-3,4-dihydroisoquinolin-1(2H)-one (49.7 mg, 0.140 mmol) and 1-(2-(tetrahydro-2H-pyran-4-yl)-5-(4,4,5,5-tetramethyl-1,3,2-dioxaborolan-2-yl)benzyl)azetidine (65 mg, 0.182 mmol) to give the title compound as a beige solid (38, 51% yield). ¹H NMR (500 MHz, DMSO-d₆) δ 8.1-8.2 (m, 1H), 7.7-7.8 (m, 1H), 7.6-7.7 (m, 2H), 7.5-7.5 (m, 1H), 7.3-7.4 (m, 1H), 6.8-6.9 (m, 2H), 3.9-4.0 (m, 2H), 3.6-3.7 (m, 2H), 3.5-3.5 (m, 2H), 3.4-3.5 (m, 2H), 3.1-3.2 (m, 5H), 2.9-3.0 (m, 2H), 2.0-2.0 (m, 2H), 1.7-1.8 (m, 2H), 1.6-1.7 (m, 2H); LCMS: [M+H]+=506.57

### Example 171: 6-(3-amino-6-(3-(azetidin-1-ylmethyl)-4-(tetrahydro-2H-pyran-4-yl)phenyl)-5-fluoropyrazin-2-yl)-8-fluoro-3,4-dihydroisoquinolin-1(2H)-one (I-171)

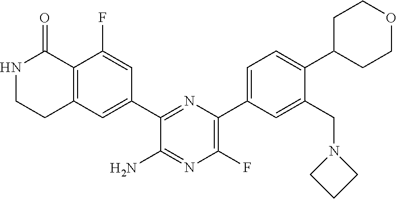 [SMILES: Nc1nc(F)c(-c2ccc(C3CCOCC3)c(CN3CCC3)c2)nc1-c1cc(F)c2c(c1)CCNC2=O]

Prepared from 6-(3-amino-6-bromo-5-fluoropyrazin-2-yl)-8-fluoro-3,4-dihydroisoquinolin-1(2H)-one (49.7 mg, 0.140 mmol) and 1-(2-(tetrahydro-2H-pyran-4-yl)-5-(4,4,5,5-tetramethyl-1,3,2-dioxaborolan-2-yl)benzyl)azetidine (65 mg, 0.182 mmol) to give the title compound as a pale-yellow solid (41, 55% yield). 1H NMR (500 MHz, DMSO-d₆) δ 8.0-8.1 (m, 1H), 7.78 (s, 1H), 7.7-7.7 (m, 1H), 7.5-7.5 (m, 1H), 7.4-7.5 (m, 1H), 7.37 (d, 1H, J=8.2 Hz), 6.9-7.0 (m, 2H), 3.9-4.0 (m, 2H), 3.6-3.7 (m, 2H), 3.5-3.5 (m, 2H), 3.38 (br s, 2H), 3.1-3.2 (m, 4H), 3.1-3.1 (m, 1H), 2.9-3.0 (m, 2H), 2.0-2.0 (m, 2H), 1.7-1.8 (m, 2H), 1.6-1.7 (m, 2H); LCMS: [M+H]⁺=506.57.

### Example 172: (R)-6-(3-amino-6-(3-((dimethylamino)methyl)-4-(2-methylmorpholino)phenyl)-5-fluoropyrazin-2-yl)-3,4-dihydroisoquinolin-1(2H)-one (I-172)

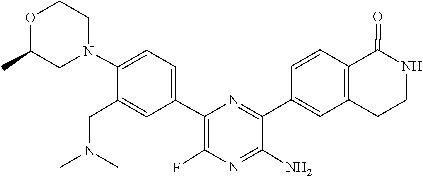 [SMILES: C[C@@H]1CN(c2ccc(-c3nc(-c4ccc5c(c4)CCNC5=O)c(N)nc3F)cc2CN(C)C)CCO1]

Prepared from 6-(3-amino-6-bromo-5-fluoropyrazin-2-yl)-3,4-dihydroisoquinolin-1(2H)-one (51.3 mg, 0.152 mmol) and (R)—N,N-dimethyl-1-(2-(2-methylmorpholino)-5-(4,4,5,5-tetramethyl-1,3,2-dioxaborolan-2-yl)phenyl)methanamine (75 mg, 0.198 mmol, prepared from a sequence analogous to previous examples, starting from 5-bromo-2-fluorobenzaldehyde and (R)-2-methyl-morpholine hydrochloride) to give the title compound as a pale-yellow solid (33 mg, 42% yield). ¹H NMR (500 MHz, DMSO-d₆) δ=8.05-7.98 (m, 1H), 7.98-7.93 (m, 1H), 7.90-7.86 (m, 1H), 7.76-7.71 (m, 1H), 7.70-7.66 (m, 1H), 7.66-7.61 (m, 1H), 7.18-7.13 (m, 1H), 6.87-6.77 (m, 2H), 3.90-3.85 (m, 1H), 3.76-3.68 (m, 2H), 3.49-3.45 (m, 2H), 3.45-3.42 (m, 2H), 3.20-3.14 (m, 1H), 3.10-3.05 (m, 1H), 3.01-2.96 (m, 2H), 2.78-2.71 (m, 1H), 2.47-2.42 (m, 1H), 2.24-2.18 (m, 6H), 1.17-1.11 (m, 3H); LCMS: [M+H]⁺=491.52.

### Example 173: (R)-6-(3-amino-6-(3-((dimethylamino)methyl)-4-(2-methylmorpholino)phenyl)-5-fluoropyrazin-2-yl)-7-fluoro-3,4-dihydroisoquinolin-1(2H)-one (I-173)

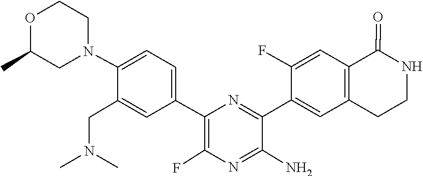 [SMILES: C[C@@H]1CN(c2ccc(-c3nc(-c4cc5c(cc4F)C(=O)NCC5)c(N)nc3F)cc2CN(C)C)CCO1]

Prepared from 6-(3-amino-6-bromo-5-fluoropyrazin-2-yl)-7-fluoro-3,4-dihydroisoquinolin-1(2H)-one (54.0 mg, 0.152 mmol) and (R)—N,N-dimethyl-1-(2-(2-methylmorpholino)-5-(4,4,5,5-tetramethyl-1,3,2-dioxaborolan-2-yl)phenyl)methanamine (75 mg, 0.198 mmol) to give the title compound as a beige solid (48 mg, 59% yield). ¹H NMR (500 MHz, DMSO-d₆) δ=8.23-8.11 (m, 1H), 7.88-7.81 (m, 1H), 7.73-7.67 (m, 1H), 7.67-7.63 (m, 1H), 7.51 (d, J=6.8 Hz, 1H), 7.16-7.11 (m, 1H), 6.78 (s, 2H), 3.90-3.84 (m, 1H), 3.77-3.67 (m, 2H), 3.48-3.40 (m, 4H), 3.18-3.12 (m, 1H), 3.08-3.03 (m, 1H), 2.95 (br t, J=6.4 Hz, 2H), 2.77-2.70 (m, 1H), 2.45 (dd, J=10.0, 11.3 Hz, 1H), 2.21-2.14 (m, 6H), 1.15-1.10 (m, 3H); LCMS: [M+H]⁺=509.51

### Example 174: 6-(3-amino-5-fluoro-6-(3-(((2-methoxyethyl)(methyl)amino)methyl)-4-(tetrahydro-2H-pyran-4-yl)phenyl)pyrazin-2-yl)-3,4-dihydroisoquinolin-1(2H)-one (I-174)

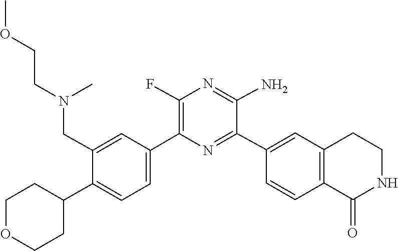 [SMILES: COCCN(C)Cc1cc(-c2nc(-c3ccc4c(c3)CCNC4=O)c(N)nc2F)ccc1C1CCOCC1]

### Step 1: N-(5-bromo-2-(tetrahydro-2H-pyran-4-yl)benzyl)-2-methoxy-N-methylethan-1-amine

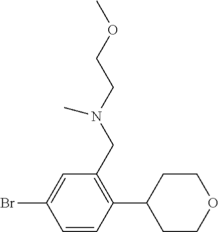 [SMILES: COCCN(C)Cc1cc(Br)ccc1C1CCOCC1]

Prepared from 5-bromo-2-(tetrahydro-2H-pyran-4-yl)benzaldehyde (400 mg, 1.486 mmol) and N-(2-methoxyethyl)methylamine (0.485 ml, 4.46 mmol) using procedures similar to those shown in earlier examples to give the product as a colorless oil (486 mg, 90% yield based on 94% purity); LCMS: [M+H]⁺=342.22

### Step 2: 2-methoxy-N-methyl-N-(2-(tetrahydro-2H-pyran-4-yl)-5-(4,4,5,5-tetramethyl-1,3,2-dioxaborolan-2-yl)benzyl)ethan-1-amine

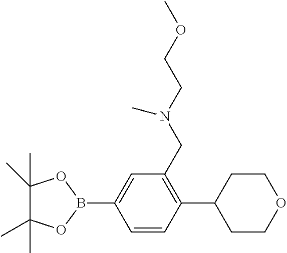 [SMILES: COCCN(C)Cc1cc(B2OC(C)(C)C(C)(C)O2)ccc1C1CCOCC1]

Prepared from N-(5-bromo-2-(tetrahydro-2H-pyran-4-yl)benzyl)-2-methoxy-N-methylethan-1-amine (190 mg, 0.555 mmol) to afford the product as a brown solid (205 mg, 95% yield); LCMS: [M+H]⁺=390.55.

### Step 3: 6-(3-amino-5-fluoro-6-(3-(((2-methoxyethyl)(methyl)amino)methyl)-4-(tetrahydro-2H-pyran-4-yl)phenyl)pyrazin-2-yl)-3,4-dihydroisoquinolin-1(2H)-one

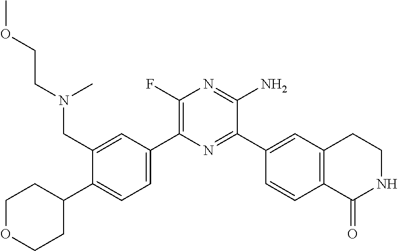 [SMILES: COCCN(C)Cc1cc(-c2nc(-c3ccc4c(c3)CCNC4=O)c(N)nc2F)ccc1C1CCOCC1]

Prepared from 6-(3-amino-6-bromo-5-fluoropyrazin-2-yl)-3,4-dihydroisoquinolin-1(2H)-one (41.1 mg, 0.122 mmol) and 2-methoxy-N-methyl-N-(2-(tetrahydro-2H-pyran-4-yl)-5-(4,4,5,5-tetramethyl-1,3,2-dioxaborolan-2-yl)benzyl)ethan-1-amine (65 mg, 0.159 mmol) to give the title compound as a pale-grey solid (48, 72% yield). ¹H NMR (500 MHz, DMSO-d₆) δ=8.04-7.98 (m, 1H), 7.96 (d, J=7.9 Hz, 1H), 7.78-7.74 (m, 1H), 7.73-7.71 (m, 1H), 7.70-7.67 (m, 1H), 7.67-7.63 (m, 1H), 7.43-7.38 (m, 1H), 6.87 (br s, 2H), 4.01-3.94 (m, 2H), 3.62-3.56 (m, 2H), 3.50-3.42 (m, 6H), 3.29-3.26 (m, 1H), 3.23 (s, 3H), 3.03-2.97 (m, 2H), 2.58-2.55 (m, 2H), 2.18-2.12 (m, 3H), 1.77-1.68 (m, 2H), 1.66-1.60 (m, 2H); LCMS: [M+H]⁺=520.61.

### Example 175: 6-(3-amino-5-fluoro-6-(3-(((2-methoxyethyl)(methyl)amino)methyl)-4-(tetrahydro-2H-pyran-4-yl)phenyl)pyrazin-2-yl)-7-fluoro-3,4-dihydroisoquinolin-1(2H)-one (I-175)

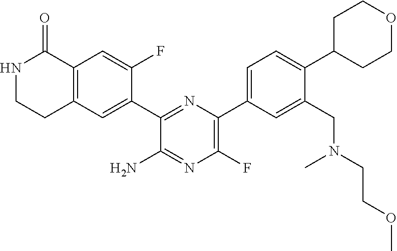 [SMILES: COCCN(C)Cc1cc(-c2nc(-c3cc4c(cc3F)C(=O)NCC4)c(N)nc2F)ccc1C1CCOCC1]

Prepared from 6-(3-amino-6-bromo-5-fluoropyrazin-2-yl)-7-fluoro-3,4-dihydroisoquinolin-1(2H)-one (43.3 mg, 0.122 mmol) and 2-methoxy-N-methyl-N-(2-(tetrahydro-2H-pyran-4-yl)-5-(4,4,5,5-tetramethyl-1,3,2-dioxaborolan-2-yl)benzyl)ethan-1-amine (65 mg, 0.159 mmol) to give the title compound as a beige solid (39, 56.5% yield). ¹H NMR (500 MHz, DMSO-d₆) δ=8.20-8.14 (m, 1H), 7.74-7.69 (m, 1H), 7.69-7.67 (m, 1H), 7.66-7.62 (m, 1H), 7.52 (d, J=6.8 Hz, 1H), 7.41-7.34 (m, 1H), 6.89-6.75 (m, 2H), 4.00-3.94 (m, 2H), 3.59-3.53 (m, 2H), 3.49-3.42 (m, 6H), 3.29-3.26 (m, 1H), 3.25-3.18 (m, 3H), 2.99-333.Neo2.94 (m, 2H), 2.57-2.54 (m, 2H), 2.18-2.10 (m, 3H), 1.76-1.67 (m, 2H), 1.66-1.59 (m, 2H); LCMS: [M+H]⁺=538.53.

### Example 176: 6-(3-amino-5-fluoro-6-(3-(((2-methoxyethyl)(methyl)amino)methyl)-4-(tetrahydro-2H-pyran-4-yl)phenyl)pyrazin-2-yl)-8-fluoro-3,4-dihydroisoquinolin-1(2H)-one (I-176)

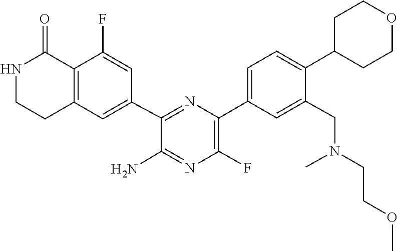 [SMILES: COCCN(C)Cc1cc(-c2nc(-c3cc(F)c4c(c3)CCNC4=O)c(N)nc2F)ccc1C1CCOCC1]

Prepared from 6-(3-amino-6-bromo-5-fluoropyrazin-2-yl)-8-fluoro-3,4-dihydroisoquinolin-1(2H)-one (43.3 mg, 0.122 mmol) and 2-methoxy-N-methyl-N-(2-(tetrahydro-2H-pyran-4-yl)-5-(4,4,5,5-tetramethyl-1,3,2-dioxaborolan-2-yl)benzyl)ethan-1-amine (65 mg, 0.159 mmol) to give the title compound as a beige solid (41, 59% yield). ¹H NMR (500 MHz, DMSO-d₆) δ=8.09-8.02 (m, 1H), 7.78-7.74 (m, 1H), 7.73-7.69 (m, 1H), 7.53-7.49 (m, 1H), 7.45-7.38 (m, 2H), 7.04-6.92 (m, 2H), 4.00-3.95 (m, 2H), 3.58 (s, 2H), 3.49-3.43 (m, 4H), 3.39-3.37 (m, 2H), 3.28 (br d, J=2.4 Hz, 1H), 3.23 (s, 3H), 3.01-2.94 (m, 2H), 2.58-2.56 (m, 2H), 2.18-2.13 (m, 3H), 1.77-1.68 (m, 2H), 1.67-1.61 (m, 2H); LCMS: [M+H]⁺=538.59.

### Example 177: (R)-6-(3-amino-6-(3-((dimethylamino)methyl)-4-(2-methylmorpholino)phenyl)-5-fluoropyrazin-2-yl)-8-fluoro-3,4-dihydroisoquinolin-1(2H)-one (I-177)

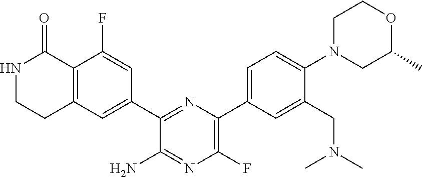 [SMILES: C[C@@H]1CN(c2ccc(-c3nc(-c4cc(F)c5c(c4)CCNC5=O)c(N)nc3F)cc2CN(C)C)CCO1]

Prepared from 6-(3-amino-6-bromo-5-fluoropyrazin-2-yl)-8-fluoro-3,4-dihydroisoquinolin-1(2H)-one (54.0 mg, 0.152 mmol) and (R)—N,N-dimethyl-1-(2-(2-methylmorpholino)-5-(4,4,5,5-tetramethyl-1,3,2-dioxaborolan-2-yl)phenyl)methanamine (75 mg, 0.198 mmol) to give the title compound as a yellow solid (20 mg, 25% yield). ¹H NMR (500 MHz, DMSO-d₆) δ=8.08-7.99 (m, 1H), 7.87 (br s, 1H), 7.77-7.70 (m, 1H), 7.52-7.47 (m, 1H), 7.45-7.39 (m, 1H), 7.19-7.13 (m, 1H), 6.96-6.87 (m, 2H), 3.90-3.85 (m, 1H), 3.76-3.70 (m, 2H), 3.49-3.46 (m, 2H), 3.19-3.15 (m, 1H), 3.10-3.06 (m, 1H), 3.01-2.96 (m, 2H), 2.79-2.70 (m, 2H), 2.46-2.41 (m, 2H), 2.24-2.17 (m, 6H), 1.16-1.10 (m, 3H); LCMS: [M+H]⁺=509.57.

### Example 178: 6-(3-amino-6-(3-((dimethylamino)methyl)-4-((tetrahydro-2H-pyran-4-yl)methoxy)phenyl)-5-fluoropyrazin-2-yl)-3,4-dihydroisoquinolin-1(2H)-one (I-178)

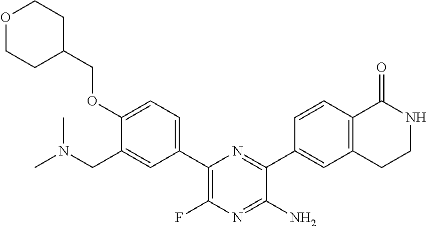 [SMILES: CN(C)Cc1cc(-c2nc(-c3ccc4c(c3)CCNC4=O)c(N)nc2F)ccc1OCC1CCOCC1]

### Step 1: Preparation of 1-(5-bromo-2-((tetrahydro-2H-pyran-4-yl)methoxy)phenyl)-N,N-dimethylmethanamine

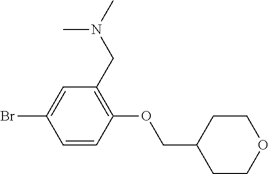 [SMILES: CN(C)Cc1cc(Br)ccc1OCC1CCOCC1]

Prepared from 5-bromo-2-((tetrahydro-2H-pyran-4-yl)methoxy)benzaldehyde (400 mg, 0.882 mmol) and dimethylamine, 2.0M solution in THF (1.324 ml, 2.65 mmol) to give the product as a white solid (320 mg, 99%, based on 90% purity). LCMS: [M+H]⁺=328.37.

### Step 2: N,N-dimethyl-1-(2-((tetrahydro-2H-pyran-4-yl)methoxy)-5-(4,4,5,5-tetramethyl-1,3,2-dioxaborolan-2-yl)phenyl)methanamine

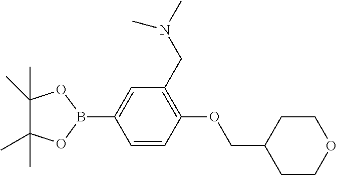 [SMILES: CN(C)Cc1cc(B2OC(C)(C)C(C)(C)O2)ccc1OCC1CCOCC1]

Prepared from 1-(5-bromo-2-((tetrahydro-2H-pyran-4-yl)methoxy)phenyl)-N,N-dimethylmethanamine (210 mg, 0.576 mmol) to give the product which was isolated as a brown solid (205 mg, 95% yield based on 95% purity); LCMS: [M+H]⁺=376.51.

### Step 3: 6-(3-amino-6-(3-((dimethylamino)methyl)-4-((tetrahydro-2H-pyran-4-yl)methoxy)phenyl)-5-fluoropyrazin-2-yl)-3,4-dihydroisoquinolin-1(2H)-one

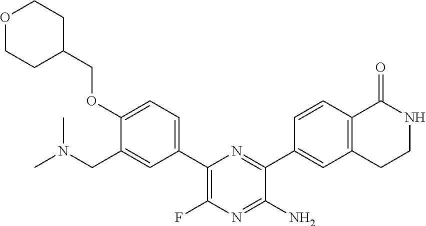 [SMILES: CN(C)Cc1cc(-c2nc(-c3ccc4c(c3)CCNC4=O)c(N)nc2F)ccc1OCC1CCOCC1]

Prepared from 6-(3-amino-6-bromo-5-fluoropyrazin-2-yl)-3,4-dihydroisoquinolin-1(2H)-one (46.0 mg, 0.136 mmol) and N,N-dimethyl-1-(2-((tetrahydro-2H-pyran-4-yl)methoxy)-5-(4,4,5,5-tetramethyl-1,3,2-dioxaborolan-2-yl)phenyl)methanamine (70 mg, 0.177 mmol) to give the title compound as a beige solid. (54 mg, 74% yield). ¹H NMR (500 MHz, DMSO-d₆) δ 8.0-8.0 (m, 1H), 7.9-8.0 (m, 1H), 7.8-7.8 (m, 1H), 7.7-7.8 (m, 1H), 7.7-7.7 (m, 1H), 7.6-7.7 (m, 1H), 7.0-7.1 (m, 1H), 6.77 (s, 2H), 3.9-3.9 (m, 4H), 3.4-3.5 (m, 6H), 3.0-3.0 (m, 2H), 2.2-2.2 (m, 6H), 2.0-2.1 (m, 1H), 1.7-1.7 (m, 2H), 1.4-1.4 (m, 2H); LCMS: [M+H]⁺=506.63.

### Example 179: 6-(3-amino-6-(3-((dimethylamino)methyl)-4-((tetrahydro-2H-pyran-4-yl)methoxy)phenyl)-5-fluoropyrazin-2-yl)-7-fluoro-3,4-dihydroisoquinolin-1(2H)-one (I-179)

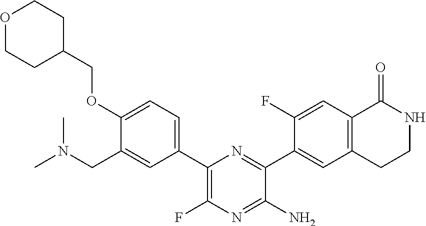 [SMILES: CN(C)Cc1cc(-c2nc(-c3cc4c(cc3F)C(=O)NCC4)c(N)nc2F)ccc1OCC1CCOCC1]

Prepared from 6-(3-amino-6-bromo-5-fluoropyrazin-2-yl)-7-fluoro-3,4-dihydroisoquinolin-1(2H)-one (48.4 mg, 0.136 mmol) and N,N-dimethyl-1-(2-((tetrahydro-2H-pyran-4-yl)methoxy)-5-(4,4,5,5-tetramethyl-1,3,2-dioxaborolan-2-yl)phenyl)methanamine (70 mg, 0.177 mmol) to give the title compound as a beige solid. (22 mg, 29% yield). ¹H NMR (500 MHz, DMSO-d₆) δ=8.22-8.12 (m, 1H), 7.81-7.75 (m, 1H), 7.71-7.66 (m, 1H), 7.64 (d, J=10.1 Hz, 1H), 7.53-7.48 (m, 1H), 7.07-7.02 (m, 1H), 6.78-6.69 (m, 2H), 3.94-3.86 (m, 4H), 3.48-3.41 (m, 4H), 3.39-3.36 (m, 2H), 2.99-2.92 (m, 2H), 2.22-2.15 (m, 6H), 2.07-1.98 (m, 1H), 1.73-1.67 (m, 2H), 1.44-1.34 (m, 2H); LCMS: [M+H]⁺=524.62.

### Example 180: 6-(3-amino-6-(3-((dimethylamino)methyl)-4-((tetrahydro-2H-pyran-4-yl)methoxy)phenyl)-5-fluoropyrazin-2-yl)-8-fluoro-3,4-dihydroisoquinolin-1(2H)-one (I-180)

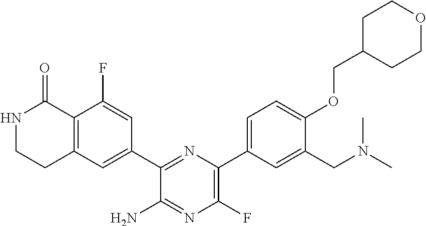 [SMILES: CN(C)Cc1cc(-c2nc(-c3cc(F)c4c(c3)CCNC4=O)c(N)nc2F)ccc1OCC1CCOCC1]

Prepare from 6-(3-amino-6-bromo-5-fluoropyrazin-2-yl)-8-fluoro-3,4-dihydroisoquinolin-1(2H)-one (48.4 mg, 0.136 mmol) and N,N-dimethyl-1-(2-((tetrahydro-2H-pyran-4-yl)methoxy)-5-(4,4,5,5-tetramethyl-1,3,2-dioxaborolan-2-yl)phenyl)methanamine (70 mg, 0.177 mmol) to give the title compound as a pale-yellow solid (40 mg, 53% yield). ¹H NMR (DMSO-d6, 500 MHz) δ=8.10-8.01 (m, 1H), 7.85-7.79 (m, 1H), 7.77-7.69 (m, 1H), 7.52-7.47 (m, 1H), 7.45-7.39 (m, 1H), 7.10-7.04 (m, 1H), 6.86-6.86 (m, 1H), 6.94-6.80 (m, 1H), 3.95-3.88 (m, 4H), 3.48-3.44 (m, 2H), 3.41-3.36 (m, 4H), 3.01-2.94 (m, 2H), 2.24-2.17 (m, 6H), 2.10-1.99 (m, 1H), 1.76-1.67 (m, 2H), 1.45-1.35 (m, 2H); LCMS: [M+H]⁺=524.55.

### Example 181: 6-(3-amino-5-fluoro-6-(4-(tetrahydro-2H-pyran-4-yl)phenyl)pyrazin-2-yl)-4-methylisoquinolin-1(2H)-one (I-181)

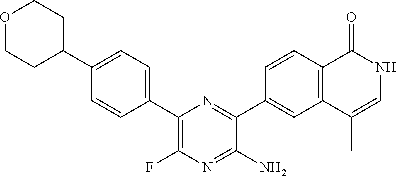 [SMILES: Cc1c[nH]c(=O)c2ccc(-c3nc(-c4ccc(C5CCOCC5)cc4)c(F)nc3N)cc12]

Prepared from 6-(3-amino-6-bromo-5-fluoropyrazin-2-yl)-4-methylisoquinolin-1(2H)-one (50 mg, 0.143 mmol) and 4-(4-tetrahydropyranyl)phenylboronic acid pinacol ester (49.5 mg, 0.172 mmol) to give the title compound (41.3 mg, 67.0% yield) as a yellow solid. ¹H NMR (500 MHz, DMSO-d₆) δ ppm 11.14 (d, J=5.13 Hz, 1H), 8.33 (d, J=8.19 Hz, 1H), 7.99 (s, 1H), 7.76-7.86 (m, 3H), 7.36 (d, J=8.19 Hz, 2H), 7.07 (d, J=5.38 Hz, 1H), 6.97 (s, 2H), 3.91-3.98 (m, 2H), 3.44 (td, J=10.88, 3.30 Hz, 2H), 2.80 (tt, J=10.36, 5.17 Hz, 1H), 2.26 (s, 3H), 1.63-1.75 (m, 4H); LCMS: [M+H]⁺=431.42.

### Example 182: 6-(3-amino-5-fluoro-6-(4-(4-isopropylpiperazin-1-yl)phenyl)pyrazin-2-yl)-7-fluoro-3-methylisoquinolin-1(2H)-one (I-182)

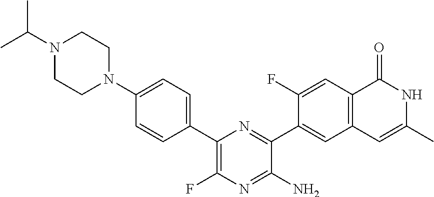 [SMILES: Cc1cc2cc(-c3nc(-c4ccc(N5CCN(C(C)C)CC5)cc4)c(F)nc3N)c(F)cc2c(=O)[nH]1]

### Step 1: 6-bromo-7-fluoro-3-methylisoquinolin-1(2H)-one

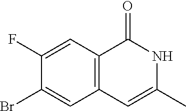

To a RBF was added copper(I) bromide (0.068 g, 0.472 mmol), Cs₂CO₃ (3.07 g, 9.43 mmol), 2,4-dibromo-5-fluorobenzamide (1.40 g, 4.72 mmol), propan-2-one (1.731 ml, 23.58 mmol) and dimethylsulfoxide (20 ml). The reaction was stirred and heated at 80° C. overnight. The reaction mixture was partitioned between brine (200 ml) and DCM (200 ml). The organic layer was separated, and the aqueous layer washed with DCM (2×100 ml). The combined organic layers were washed with brine, dried over anhydrous Na₂SO₄, and concentrated onto celite. The mixture was purified by flash sgc eluting with 0-10% MeOH/DCM+1% NH₄OH. The desired fractions were collected, concentrated and dried under vacuum to afford the product (489 mg, 40.5% yield) as a beige solid. LCMS: [M+H]⁺=256.21.

### Step 2: (7-fluoro-3-methyl-1-oxo-1,2-dihydroisoquinolin-6-yl)boronic acid

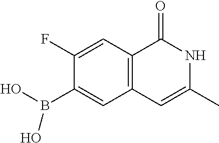 [SMILES: Cc1cc2cc(B(O)O)c(F)cc2c(=O)[nH]1]

Prepared from 6-bromo-7-fluoro-3-methylisoquinolin-1(2H)-one (700 mg, 2.73 mmol) to give the boronic acid which was used in the next step without further purification. LCMS: [M−H]⁻=220.10.

### Step 3: 6-(3-amino-6-bromo-5-fluoropyrazin-2-yl)-7-fluoro-3-methylisoquinolin-1(2H)-one

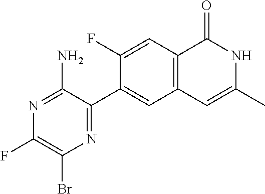 [SMILES: Cc1cc2cc(-c3nc(Br)c(F)nc3N)c(F)cc2c(=O)[nH]1]

Prepared from 5-bromo-6-fluoro-3-iodopyrazin-2-amine (350 mg, 1.101 mmol) and (7-fluoro-3-methyl-1-oxo-1,2-dihydroisoquinolin-6-yl)boronic acid (292 mg, 1.321 mmol) to give the product (104 mg, 25.7% yield). LCMS: [M+H]⁺=367.23.

### Step 4: 6-(3-amino-5-fluoro-6-(4-(4-isopropylpiperazin-1-yl)phenyl)pyrazin-2-yl)-7-fluoro-3-methylisoquinolin-1(2H)-one

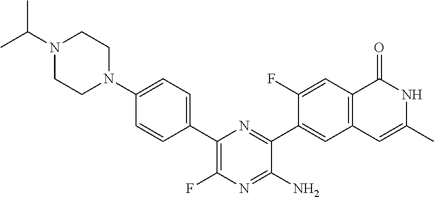 [SMILES: Cc1cc2cc(-c3nc(-c4ccc(N5CCN(C(C)C)CC5)cc4)c(F)nc3N)c(F)cc2c(=O)[nH]1]

Prepared from 6-(3-amino-6-bromo-5-fluoropyrazin-2-yl)-7-fluoro-3-methylisoquinolin-1(2H)-one (30 mg, 0.082 mmol) and 4-(4-isopropylpiperazinyl)phenylboronic acid, pinacol ester (32.4 mg, 0.098 mmol) to give the title compound (9.1 mg, 22.7% yield) as a yellow solid. ¹H NMR (500 MHz, DMSO-d₆) δ ppm 11.43 (s, 1H), 7.88 (d, J=10.27 Hz, 1H), 7.76 (d, J=6.85 Hz, 1H), 7.70 (d, J=8.31 Hz, 2H), 6.99 (d, J=8.80 Hz, 2H), 6.69 (s, 2H), 6.43 (s, 1H), 3.15-3.20 (m, 4H), 2.64-2.71 (m, 1H), 2.55-2.59 (m, 4H), 2.23 (s, 3H), 1.00 (d, J=6.48 Hz, 6H); LCMS: [M+H]⁺=491.56.

### Example 183: 6-(3-amino-6-(3-((dimethylamino)methyl)-4-morpholinophenyl)-5-fluoropyrazin-2-yl)-7-fluoro-3-methylisoquinolin-1(2H)-one (I-183)

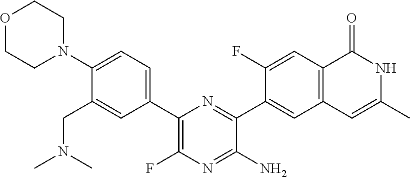 [SMILES: Cc1cc2cc(-c3nc(-c4ccc(N5CCOCC5)c(CN(C)C)c4)c(F)nc3N)c(F)cc2c(=O)[nH]1]

Prepared from 6-(3-amino-6-bromo-5-fluoropyrazin-2-yl)-7-fluoro-3-methylisoquinolin-1(2H)-one (30 mg, 0.082 mmol) and N,N-dimethyl-1-(2-morpholino-5-(4,4,5,5-tetramethyl-1,3,2-dioxaborolan-2-yl)phenyl)methanamine (34.0 mg, 0.098 mmol) to give the title compound (4.1 mg, 9.9% yield) as a green-yellow solid. ¹H NMR (500 MHz, DMSO-d₆) δ ppm 11.44 (s, 1H), 7.88 (d, J=10.15 Hz, 1H), 7.84 (s, 1H), 7.76 (d, J=6.72 Hz, 1H), 7.69 (d, J=8.31 Hz, 1H), 7.15 (d, J=8.44 Hz, 1H), 6.80 (br. s., 2H), 6.43 (s, 1H), 3.72-3.77 (m, 4H), 3.44 (s, 2H), 2.93 (br. s., 4H), 2.23 (s, 3H), 2.16 (s, 6H); LCMS [M+H]⁺=507.57.

### Example 184: 6-(3-amino-6-(3-((dimethylamino)methyl)-4-(tetrahydro-2H-pyran-4-yl)phenyl)-5-fluoropyrazin-2-yl)-7-fluoro-3-methylisoquinolin-1(2H)-one (I-184)

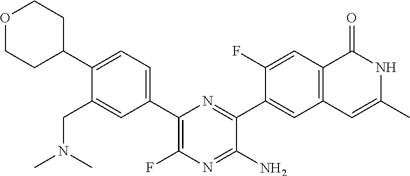 [SMILES: Cc1cc2cc(-c3nc(-c4ccc(C5CCOCC5)c(CN(C)C)c4)c(F)nc3N)c(F)cc2c(=O)[nH]1]

Prepared from 6-(3-amino-6-bromo-5-fluoropyrazin-2-yl)-7-fluoro-3-methylisoquinolin-1(2H)-one (30 mg, 0.082 mmol) and N,N-dimethyl-1-(2-(tetrahydro-2H-pyran-4-yl)-5-(4,4,5,5-tetramethyl-1,3,2-dioxaborolan-2-yl)phenyl)methanamine (33.9 mg, 0.098 mmol) to give the title compound (5.7 mg, 13.8% yield) as a grey solid. ¹H NMR (500 MHz, DMSO-d₆) δ ppm 11.44 (s, 1H), 7.88 (d, J=10.15 Hz, 1H), 7.76 (d, J=6.72 Hz, 1H), 7.70 (d, J=8.19 Hz, 1H), 7.68 (s, 1H), 7.38 (d, J=8.19 Hz, 1H), 6.85 (s, 2H), 6.43 (s, 1H), 3.95 (dd, J=10.82, 3.36 Hz, 2H), 3.41-3.47 (m, 4H), 3.17-3.24 (m, 1H), 2.23 (s, 3H), 2.15 (s, 6H), 1.70 (qd, J=12.21, 3.97 Hz, 2H), 1.57-1.65 (m, 2H); LCMS: [M+H]⁺=506.57.

### Example 185: 6-(3-amino-5-fluoro-6-(4-morpholino-3-(piperidin-1-ylmethyl)phenyl)pyrazin-2-yl)-3,4-dihydroisoquinolin-1(2H)-one (I-185)

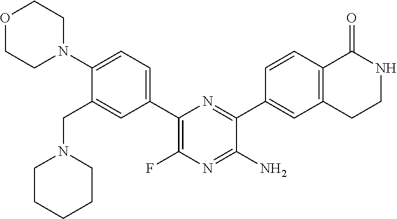 [SMILES: Nc1nc(F)c(-c2ccc(N3CCOCC3)c(CN3CCCCC3)c2)nc1-c1ccc2c(c1)CCNC2=O]

Prepared from 6-(3-amino-6-bromo-5-fluoropyrazin-2-yl)-3,4-dihydroisoquinolin-1(2H)-one (41.5 mg, 0.123 mmol) and 4-(2-(piperidin-1-ylmethyl)-4-(4,4,5,5-tetramethyl-1,3,2-dioxaborolan-2-yl)phenyl)morpholine (65 mg, 0.160 mmol, in turn prepared from a sequence starting from 5-bromo-2-morpholinobenzaldehyde and piperidine using methods similar to previous examples) to give the title compound as a beige solid (34 mg, 51% yield). ¹H NMR (500 MHz, DMSO-d₆) δ=8.04-7.98 (m, 1H), 7.97-7.92 (m, 2H), 7.76-7.69 (m, 2H), 7.68-7.65 (m, 1H), 7.22-7.14 (m, 1H), 6.90-6.80 (m, 2H), 3.80-3.74 (m, 4H), 3.55-3.50 (m, 2H), 3.46-3.43 (m, 2H), 3.02-2.94 (m, 6H), 2.45-2.34 (m, 4H), 1.53-1.46 (m, 4H), 1.40 (br s, 2H); LCMS: [M+H]⁺=517.60.

### Example 186: 6-(3-amino-5-fluoro-6-(4-morpholino-3-(piperidin-1-ylmethyl)phenyl)pyrazin-2-yl)-7-fluoro-3,4-dihydroisoquinolin-1(2H)-one (I-186)

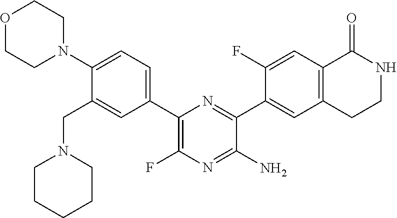 [SMILES: Nc1nc(F)c(-c2ccc(N3CCOCC3)c(CN3CCCCC3)c2)nc1-c1cc2c(cc1F)C(=O)NCC2]

Prepared from 6-(3-amino-6-bromo-5-fluoropyrazin-2-yl)-7-fluoro-3,4-dihydroisoquinolin-1(2H)-one (43.7 mg, 0.123 mmol) and 4-(2-(piperidin-1-ylmethyl)-4-(4,4,5,5-tetramethyl-1,3,2-dioxaborolan-2-yl)phenyl)morpholine (65 mg, 0.160 mmol) to give the title compound as a beige solid (30 mg, 43% yield). ¹H NMR (500 MHz, DMSO-d₆) δ=8.20-8.13 (m, 1H), 7.89-7.84 (m, 1H), 7.71-7.67 (m, 1H), 7.67-7.63 (m, 1H), 7.55-7.50 (m, 1H), 7.16 (d, J=8.4 Hz, 1H), 6.80 (s, 2H), 3.77-3.73 (m, 4H), 3.53-3.47 (m, 2H), 3.46-3.41 (m, 2H), 2.99-2.94 (m, 6H), 2.42-2.35 (m, 4H), 1.51-1.45 (m, 4H), 1.39 (br d, J=3.3 Hz, 2H); LCMS: [M+H]⁺=535.58.

### Example 187: 6-(3-amino-5-fluoro-6-(4-morpholino-3-(piperidin-1-ylmethyl)phenyl)pyrazin-2-yl)-8-fluoro-3,4-dihydroisoquinolin-1(2H)-one (I-187)

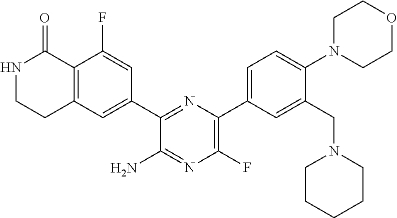 [SMILES: Nc1nc(F)c(-c2ccc(N3CCOCC3)c(CN3CCCCC3)c2)nc1-c1cc(F)c2c(c1)CCNC2=O]

Prepared from 6-(3-amino-6-bromo-5-fluoropyrazin-2-yl)-8-fluoro-3,4-dihydroisoquinolin-1(2H)-one (43.7 mg, 0.123 mmol) and 4-(2-(piperidin-1-ylmethyl)-4-(4,4,5,5-tetramethyl-1,3,2-dioxaborolan-2-yl)phenyl)morpholine (65 mg, 0.160 mmol) to give the title compound as a pale-yellow solid (35 mg, 51% yield). ¹H NMR (500 MHz, DMSO-d₆) b=8.09-8.00 (m, 1H), 7.98-7.91 (m, 1H), 7.77-7.67 (m, 1H), 7.55-7.48 (m, 1H), 7.45 (br d, J=12.0 Hz, 1H), 7.17 (d, J=8.4 Hz, 1H), 6.95 (br s, 2H), 3.75 (br d, J=3.8 Hz, 4H), 3.51-3.48 (m, 3H), 3.40-3.37 (m, 2H), 3.00-2.93 (m, 6H), 2.40 (br s, 4H), 1.53-1.44 (m, 4H), 1.44-1.37 (m, 2H); LCMS: [M+H]⁺=535.52.

### Example 188: 6-(3-amino-5-fluoro-6-(3-((3-methoxyazetidin-1-yl)methyl)-4-morpholinophenyl)pyrazin-2-yl)-3,4-dihydroisoquinolin-1(2H)-one (I-188)

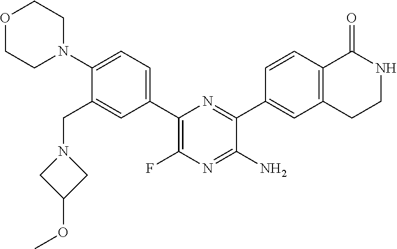 [SMILES: COC1CN(Cc2cc(-c3nc(-c4ccc5c(c4)CCNC5=O)c(N)nc3F)ccc2N2CCOCC2)C1]

Prepared from 6-(3-amino-6-bromo-5-fluoropyrazin-2-yl)-3,4-dihydroisoquinolin-1(2H)-one (53.6 mg, 0.159 mmol) and 4-(2-((3-methoxyazetidin-1-yl)methyl)-4-(4,4,5,5-tetramethyl-1,3,2-dioxaborolan-2-yl)phenyl)morpholine (65 mg, 0.159 mmol, in turn prepared using a sequence starting from 5-bromo-2-morpholinobenzaldehyde and 3-methoxyazetidine similar to previous examples) to give the title compound as a beige solid (31 mg, 36% yield). ¹H NMR (500 MHz, DMSO-d₆) δ 8.0-8.0 (m, 1H), 7.96 (br d, 1H, J=7.9 Hz), 7.8-7.9 (m, 1H), 7.7-7.8 (m, 2H), 7.66 (s, 1H), 7.1-7.2 (m, 1H), 6.8-6.9 (m, 2H), 3.98 (quin, 1H, J=5.5 Hz), 3.7-3.8 (m, 4H), 3.68 (s, 2H), 3.5-3.5 (m, 2H), 3.4-3.5 (m, 2H), 3.1-3.2 (m, 3H), 3.0-3.0 (m, 2H), 2.91 (br s, 6H); LCMS: [M+H]⁺=519.60.

### Example 189: 6-(3-amino-5-fluoro-6-(3-((3-methoxyazetidin-1-yl)methyl)-4-morpholinophenyl)pyrazin-2-yl)-7-fluoro-3,4-dihydroisoquinolin-1(2H)-one (I-189)

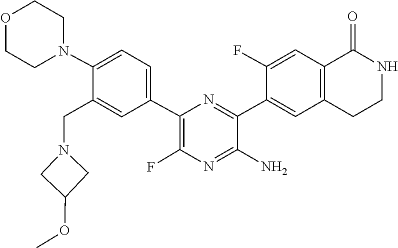 [SMILES: COC1CN(Cc2cc(-c3nc(-c4cc5c(cc4F)C(=O)NCC5)c(N)nc3F)ccc2N2CCOCC2)C1]

Prepared from 6-(3-amino-6-bromo-5-fluoropyrazin-2-yl)-7-fluoro-3,4-dihydroisoquinolin-1(2H)-one (45.2 mg, 0.127 mmol) and 4-(2-((3-methoxyazetidin-1-yl)methyl)-4-(4,4,5,5-tetramethyl-1,3,2-dioxaborolan-2-yl)phenyl)morpholine (65 mg, 0.159 mmol) to give the title compound as a beige solid (34 mg, 47% yield). ¹H NMR (500 MHz, DMSO-d₆) δ 8.1-8.2 (m, 1H), 7.8-7.8 (m, 1H), 7.6-7.7 (m, 2H), 7.5-7.5 (m, 1H), 7.1-7.2 (m, 1H), 6.80 (s, 2H), 3.9-4.0 (m, 1H), 3.7-3.8 (m, 4H), 3.6-3.7 (m, 2H), 3.5-3.5 (m, 2H), 3.43 (br s, 2H), 3.1-3.2 (m, 3H), 2.9-3.0 (m, 2H), 2.8-2.9 (m, 6H); LCMS: [M+H]⁺=537.53.

### Example 190: 6-(3-amino-5-fluoro-6-(3-((3-methoxyazetidin-1-yl)methyl)-4-(tetrahydro-2H-pyran-4-yl)phenyl)pyrazin-2-yl)-7-fluoro-3,4-dihydroisoquinolin-1(2H)-one (I-190)

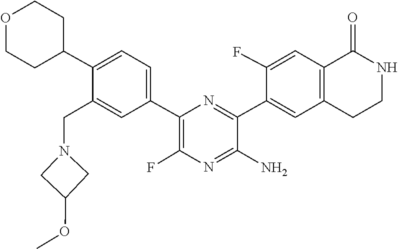 [SMILES: COC1CN(Cc2cc(-c3nc(-c4cc5c(cc4F)C(=O)NCC5)c(N)nc3F)ccc2C2CCOCC2)C1]

Prepared from 6-(3-amino-6-bromo-5-fluoropyrazin-2-yl)-7-fluoro-3,4-dihydroisoquinolin-1(2H)-one (43.7 mg, 0.123 mmol) and 3-methoxy-1-(2-(tetrahydro-2H-pyran-4-yl)-5-(4,4,5,5-tetramethyl-1,3,2-dioxaborolan-2-yl)benzyl)azetidine (65 mg, 0.154 mmol, which was prepared from 5-bromo-2-(tetrahydro-2H-pyran-4-yl)benzaldehyde and 3-methoxyazetidine (0.101 mL, 1.115 mmol) to give the title compound as a beige solid (16, 23% yield). ¹H NMR (500 MHz, DMSO-d₆) δ 8.1-8.2 (m, 1H), 7.7-7.8 (m, 1H), 7.7-7.7 (m, 1H), 7.6-7.7 (m, 1H), 7.52 (d, 1H, J=6.7 Hz), 7.3-7.4 (m, 1H), 6.7-6.9 (m, 2H), 3.9-4.0 (m, 3H), 3.6-3.8 (m, 2H), 3.4-3.5 (m, 6H), 3.1-3.2 (m, 4H), 2.9-3.0 (m, 2H), 2.8-2.9 (m, 2H), 1.7-1.8 (m, 2H), 1.6-1.7 (m, 2H); LCMS: [M+H]⁺=536.52.

### Example 191: 6-(3-amino-5-fluoro-6-(3-((3-methoxyazetidin-1-yl)methyl)-4-(tetrahydro-2H-pyran-4-yl)phenyl)pyrazin-2-yl)-8-fluoro-3,4-dihydroisoquinolin-1(2H)-one (I-191)

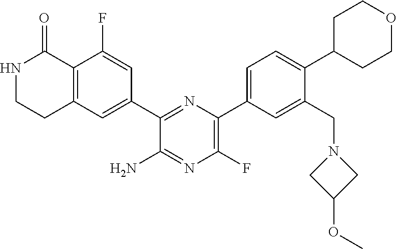 [SMILES: COC1CN(Cc2cc(-c3nc(-c4cc(F)c5c(c4)CCNC5=O)c(N)nc3F)ccc2C2CCOCC2)C1]

Prepared from 6-(3-amino-6-bromo-5-fluoropyrazin-2-yl)-8-fluoro-3,4-dihydroisoquinolin-1(2H)-one (43.7 mg, 0.123 mmol) and 3-methoxy-1-(2-(tetrahydro-2H-pyran-4-yl)-5-(4,4,5,5-tetramethyl-1,3,2-dioxaborolan-2-yl)benzyl)azetidine (65 mg, 0.154 mmol to give the title compound as a beige solid (36, 52% yield). ¹H NMR (500 MHz, DMSO-d₆) δ 8.0-8.1 (m, 1H), 7.8-7.8 (m, 1H), 7.7-7.7 (m, 1H), 7.5-7.5 (m, 1H), 7.4-7.5 (m, 1H), 7.38 (br d, 1H, J=8.1 Hz), 6.9-7.0 (m, 2H), 4.0-4.0 (m, 3H), 3.73 (br s, 2H), 3.5-3.6 (m, 4H), 3.37 (br s, 2H), 3.1-3.2 (m, 3H), 3.1-3.1 (m, 1H), 3.0-3.0 (m, 2H), 2.91 (br s, 2H), 1.7-1.8 (m, 2H), 1.6-1.7 (m, 2H); LCMS: [M+H]⁺=536.59.

### Example 192: 6-(3-amino-5-fluoro-6-(3-((3-methoxyazetidin-1-yl)methyl)-4-morpholinophenyl)pyrazin-2-yl)-8-fluoro-3,4-dihydroisoquinolin-1(2H)-one (I-192)

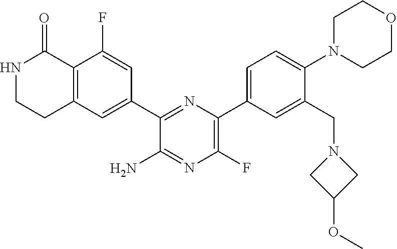 [SMILES: COC1CN(Cc2cc(-c3nc(-c4cc(F)c5c(c4)CCNC5=O)c(N)nc3F)ccc2N2CCOCC2)C1]

Prepared from 6-(3-amino-6-bromo-5-fluoropyrazin-2-yl)-8-fluoro-3,4-dihydroisoquinolin-1(2H)-one (45.2 mg, 0.127 mmol) and 4-(2-((3-methoxyazetidin-1-yl)methyl)-4-(4,4,5,5-tetramethyl-1,3,2-dioxaborolan-2-yl)phenyl)morpholine (65 mg, 0.159 mmol) to give the title compound as a pale-yellow solid (15 mg, 21% yield). ¹H NMR (500 MHz, DMSO-d₆) δ ppm 8.05 (br. s., 1H), 7.85 (s, 1H), 7.72 (d, J=8.19 Hz, 1H), 7.51 (s, 1H), 7.44 (d, J=11.86 Hz, 1H), 7.15 (d, J=8.31 Hz, 1H), 6.95 (s, 2H), 3.99 (quin, J=5.59 Hz, 1H), 3.75 (d, J=3.91 Hz, 4H), 3.68 (s, 2H), 3.53 (t, J=6.42 Hz, 2H), 3.37 (br. s., 2H), 3.15 (s, 3H), 2.98 (t, J=6.17 Hz, 2H), 2.92 (br. s., 6H); LCMS: [M+H]⁺=537.59.

### Example 193: 6-(3-amino-5-fluoro-6-(3-((3-methoxyazetidin-1-yl)methyl)-4-(tetrahydro-2H-pyran-4-yl)phenyl)pyrazin-2-yl)-3,4-dihydroisoquinolin-1(2H)-one (I-193)

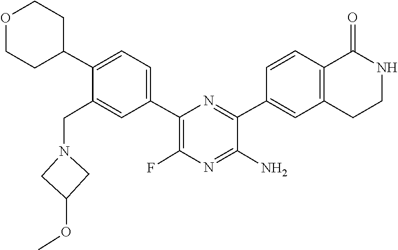 [SMILES: COC1CN(Cc2cc(-c3nc(-c4ccc5c(c4)CCNC5=O)c(N)nc3F)ccc2C2CCOCC2)C1]

Prepared from 6-(3-amino-6-bromo-5-fluoropyrazin-2-yl)-3,4-dihydroisoquinolin-1(2H)-one (41.5 mg, 0.123 mmol) and 3-methoxy-1-(2-(tetrahydro-2H-pyran-4-yl)-5-(4,4,5,5-tetramethyl-1,3,2-dioxaborolan-2-yl)benzyl)azetidine (65 mg, 0.154 mmol) to give the title compound as a beige solid (15, 22% yield). 1H NMR (500 MHz, DMSO-d₆) δ ppm 8.00 (br. s., 1H), 7.96 (d, J=7.95 Hz, 1H), 7.77 (s, 1H), 7.68-7.75 (m, 2H), 7.66 (s, 1H), 7.37 (d, J=8.19 Hz, 1H), 6.88 (s, 2H), 3.93-4.02 (m, 3H), 3.71 (s, 2H), 3.45-3.53 (m, 4H), 3.41-3.45 (m, 2H), 3.15 (s, 3H), 3.11 (t, J=11.68 Hz, 1H), 2.99 (t, J=6.36 Hz, 2H), 2.89 (t, J=6.48 Hz, 2H), 1.67-1.76 (m, 2H), 1.59-1.66 (m, 2H); LCMS: [M+H]⁺=518.47.

### Example 194: 7-(3-amino-6-(3-((dimethylamino)methyl)-4-((2R,6S)-2,6-dimethylmorpholino)phenyl)-5-fluoropyrazin-2-yl)-2-methylquinazolin-4(3H)-one (I-194)

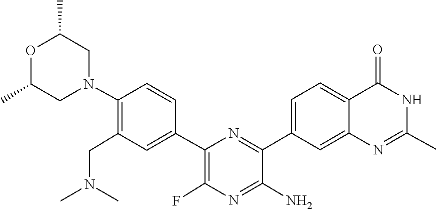

Prepared from 7-(3-amino-6-bromo-5-fluoropyrazin-2-yl)-2-methylquinazolin-4(3H)-one (40 mg, 0.114 mmol) and 1-(2-((2S,6R)-2,6-dimethylmorpholino)-5-(4,4,5,5-tetramethyl-1,3,2-dioxaborolan-2-yl)phenyl)-N,N-dimethylmethanamine hydrochloride (46.9 mg, 0.114 mmol), tetrakis(triphenylphosphine)Palladium(0) (13.20 mg, 0.011 mmol) and potassium carbonate (55.3 mg, 0.400 mmol) in 1,4-Dioxane (2 ml) and water (0.5 ml) in MW at 120° C. for 90 min to give the title compound (26.7 mg, 43.8% yield). ¹H NMR (500 MHz, DMSO-de) δ=12.27 (s, 1H), 8.17 (d, J=8.3 Hz, 1H), 8.13 (s, 1H), 7.90 (br s, 1H), 7.88 (s, 1H), 7.77 (br d, J=7.9 Hz, 2H), 7.24-7.17 (m, 1H), 6.91 (br s, 2H), 3.83-3.75 (m, 2H), 3.14-3.06 (m, 2H), 2.42-2.23 (m, 10H), 1.11 (d, J=6.2 Hz, 6H); LCMS: [M+H]⁺=518.60.

### Example 195: 6-(3-amino-6-(4-((1S,5R)-3-cyclobutyl-3-azabicyclo[3.1.0]hexan-1-yl)phenyl)-5-fluoropyrazin-2-yl)-4,4,8-trifluoro-3-methyl-3,4-dihydroisoquinolin-1(2H)-one (I-195)

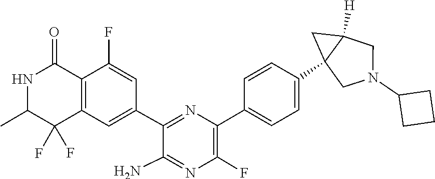 [SMILES: CC1NC(=O)c2c(F)cc(-c3nc(-c4ccc([C@]56C[C@H]5CN(C5CCC5)C6)cc4)c(F)nc3N)cc2C1(F)F]

Prepared from 6-(3-amino-6-bromo-5-fluoropyrazin-2-yl)-4,4,8-trifluoro-3-methyl-3,4-dihydroisoquinolin-1(2H)-one (30 mg, 0.074 mmol) and (1S,5R)-3-cyclobutyl-1-(4-(4,4,5,5-tetramethyl-1,3,2-dioxaborolan-2-yl)phenyl)-3-azabicyclo[3.1.0]hexane (25.1 mg, 0.074 mmol) to give the formic acid salt of the title compound (15.5 mg, 35.2% yield). ¹H NMR (500 MHz, DMSO-de) δ=8.48 (br s, 1H), 8.08 (s, 1H), 7.82-7.75 (m, 2H), 7.70 (d, J=7.6 Hz, 2H), 7.17 (d, J=8.4 Hz, 2H), 7.01 (s, 2H), 4.16-4.04 (m, 1H), 3.09-3.02 (m, 2H), 2.91 (br d, J=8.8 Hz, 1H), 2.52 (br d, J=8.6 Hz, 1H), 2.46-2.45 (m, 1H), 2.39 (br dd, J=2.0, 6.0 Hz, 1H), 1.96-1.74 (m, 5H), 1.66-1.52 (m, 2H), 1.29 (t, J=4.1 Hz, 1H), 1.23 (d, J=6.6 Hz, 3H), 0.75 (dd, J=3.8, 7.9 Hz, 1H); LCMS: [M−HCO₂H+H]⁺=538.53.

### Example 196: 7-(3-amino-6-(3-((dimethylamino)methyl)-4-((2R,6S)-2,6-dimethylmorpholino)phenyl)-5-fluoropyrazin-2-yl)-5-fluoro-2-methylquinazolin-4(3H)-one (I-196)

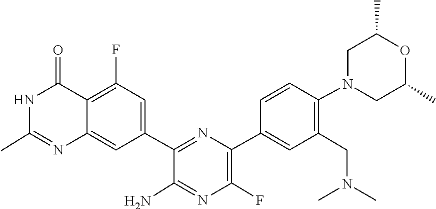

Prepared from 7-(3-amino-6-bromo-5-fluoropyrazin-2-yl)-5-fluoro-2-methylquinazolin-4(3H)-one (40 mg, 0.109 mmol) and 1-(2-((2S,6R)-2,6-dimethylmorpholino)-5-(4,4,5,5-tetramethyl-1,3,2-dioxaborolan-2-yl)phenyl)-N,N-dimethylmethanamine hydrochloride (44.6 mg, 0.109 mmol) to give the title compound (14.5 mg, 24.4% yield). ¹H NMR (500 MHz, DMSO-d₆) δ=12.40-12.25 (m, 1H), 7.87 (s, 1H), 7.74 (br d, J=8.4 Hz, 1H), 7.71 (s, 1H), 7.49 (d, J=11.6 Hz, 1H), 7.14 (d, J=8.6 Hz, 1H), 6.96 (s, 2H), 3.83-3.73 (m, 2H), 3.45 (s, 2H), 3.17 (brd, J=11.4 Hz, 2H), 2.42-2.29 (m, 5H), 2.19 (s, 6H), 1.12 (d, J=6.2 Hz, 6H); LCMS: [M+H]⁺=536.52.

### Example 197: 6-(3-amino-6-(3-((dimethylamino)methyl)-4-((2R,6S)-2,6-dimethylmorpholino)phenyl)-5-fluoropyrazin-2-yl)-8-fluoro-3,4-dihydroisoquinolin-1(2H)-one (I-197)

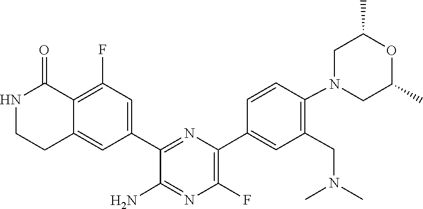 [SMILES: C[C@@H]1CN(c2ccc(-c3nc(-c4cc(F)c5c(c4)CCNC5=O)c(N)nc3F)cc2CN(C)C)C[C@H](C)O1]

Prepared from 6-(3-amino-6-bromo-5-fluoropyrazin-2-yl)-8-fluoro-3,4-dihydroisoquinolin-1(2H)-one (40 mg, 0.113 mmol) and 1-(2-((2S,6R)-2,6-dimethylmorpholino)-5-(4,4,5,5-tetramethyl-1,3,2-dioxaborolan-2-yl)phenyl)-N,N-dimethylmethanamine, hydrochloride, HCl [B] (46.3 mg, 0.113 mmol) to give the title compound (14.4 mg, 23.3% yield). ¹H NMR (500 MHz, DMSO-d6) δ=7.98 (br s, 1H), 7.81 (br s, 1H), 7.69 (br d, J=7.6 Hz, 1H), 7.42 (s, 1H), 7.35 (br d, J=11.9 Hz, 1H), 7.17-7.07 (m, 1H), 6.87 (br s, 2H), 3.79-3.68 (m, 2H), 3.20-3.55 (m, 4H) 3.05 (br d, J=10.8 Hz, 2H), 2.90 (br t, J=6.2 Hz, 2H), 2.37-1.99 (m, 8H), 1.05 (d, J=6.2 Hz, 6H); LCMS: [M+H]⁺=523.55.

### Example 198: 6-(3-amino-6-(3-((dimethylamino)methyl)-4-((2R,6S)-2,6-dimethylmorpholino)phenyl)-5-fluoropyrazin-2-yl)-3,4-dihydroisoquinolin-1(2H)-one (I-198)

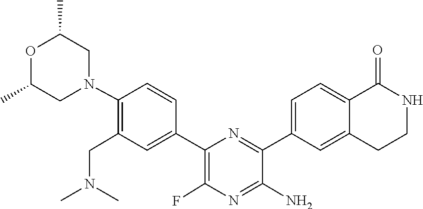 [SMILES: C[C@@H]1CN(c2ccc(-c3nc(-c4ccc5c(c4)CCNC5=O)c(N)nc3F)cc2CN(C)C)C[C@H](C)O1]

Prepared from 6-(3-amino-6-bromo-5-fluoropyrazin-2-yl)-3,4-dihydroisoquinolin-1(2H)-one (40 mg, 0.119 mmol) and 1-(2-((2S,6R)-2,6-dimethylmorpholino)-5-(4,4,5,5-tetramethyl-1,3,2-dioxaborolan-2-yl)phenyl)-N,N-dimethylmethanamine hydrochloride (48.7 mg, 0.119 mmol) to give the title compound (12.7 mg, 20.6% yield). ¹H NMR (500 MHz, DMSO-d6) δ ppm 8.00 (br. s., 1H), 7.96 (d, J=7.95 Hz, 1H), 7.88 (br. s., 1H), 7.76 (d, J=8.07 Hz, 1H), 7.67 (d, J=7.95 Hz, 1H), 7.64 (s, 1H), 7.18 (br. s., 1H), 6.84 (br. s., 2H), 3.76-3.83 (m, 2H), 3.25-3.63 (m, 4H), 3.12 (d, J=9.90 Hz, 2H), 2.99 (t, J=6.36 Hz, 2H), 2.17-2.42 (m, 8H), 1.12 (d, J=6.11 Hz, 6H); LCMS: [M+H]⁺=505.44.

### Example 199: 6-(3-amino-6-(3-(azetidin-1-ylmethyl)-4-morpholinophenyl)-5-fluoropyrazin-2-yl)-4-fluoroisoquinolin-1(2H)-one (I-199)

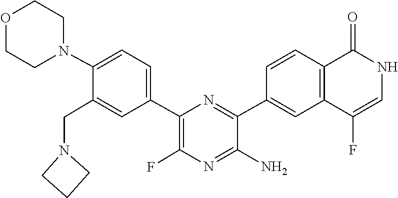 [SMILES: Nc1nc(F)c(-c2ccc(N3CCOCC3)c(CN3CCC3)c2)nc1-c1ccc2c(=O)[nH]cc(F)c2c1]

Prepared from 6-(3-amino-6-bromo-5-fluoropyrazin-2-yl)-4-fluoroisoquinolin-1(2H)-one (40 mg, 0.113 mmol) and 4-(2-(azetidin-1-ylmethyl)-4-(4,4,5,5-tetramethyl-1,3,2-dioxaborolan-2-yl)phenyl)morpholine (48.7 mg, 0.136 mmol) to give 6-(3-amino-6-(3-(azetidin-1-ylmethyl)-4-morpholinophenyl)-5-fluoropyrazin-2-yl)-4-fluoroisoquinolin-1(2H)-one (13.3 mg, 0.026 mmol, 23.27% yield) as a tan solid. ¹H NMR (500 MHz, DMSO-d₆) b ppm 11.20 (br. s., 1H), 8.32 (d, J=8.07 Hz, 1H), 8.07 (s, 1H), 7.96 (d, J=8.31 Hz, 1H), 7.88 (br. s., 1H), 7.71 (d, J=7.95 Hz, 1H), 7.45 (d, J=5.62 Hz, 1H), 7.14 (d, J=8.44 Hz, 1H), 6.96 (br. s., 2H), 3.75 (br. s., 4H), 3.61 (s, 2H), 3.17 (t, J=6.60 Hz, 4H), 2.92 (br. s., 4H), 1.98 (t, J=6.79 Hz, 2H); LCMS: [M+H]⁺=505.44.

### Example 200: 6-(3-amino-5-fluoro-6-(4-morpholino-3-(pyrrolidin-1-ylmethyl)phenyl)pyrazin-2-yl)-4-fluoroisoquinolin-1(2H)-one (I-200)

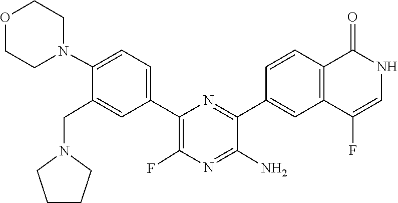 [SMILES: Nc1nc(F)c(-c2ccc(N3CCOCC3)c(CN3CCCC3)c2)nc1-c1ccc2c(=O)[nH]cc(F)c2c1]

Prepared from 6-(3-amino-6-bromo-5-fluoropyrazin-2-yl)-4-fluoroisoquinolin-1(2H)-one (40 mg, 0.113 mmol) and 4-(2-(pyrrolidin-1-ylmethyl)-4-(4,4,5,5-tetramethyl-1,3,2-dioxaborolan-2-yl)phenyl)morpholine (50.6 mg, 0.136 mmol) to give the title compound (25.4 mg, 43.2% yield) as a tan solid. ¹H NMR (500 MHz, DMSO-d₆) δ ppm 11.21 (br. s., 1H), 8.32 (d, J=7.95 Hz, 1H), 8.06 (s, 1H), 7.95 (d, J=8.44 Hz, 1H), 7.92 (s, 1H), 7.73 (d, J=8.31 Hz, 1H), 7.45 (d, J=5.62 Hz, 1H), 7.15 (d, J=8.44 Hz, 1H), 6.96 (s, 2H), 3.75 (br. s., 4H), 3.66 (s, 2H), 3.30 (s, 4H), 2.96 (br. s., 4H), 1.68 (br. s., 4H); LCMS: [M+H]⁺=519.54.

### Example 201: 6-(3-amino-5-fluoro-6-(5-morpholinopyridin-2-yl)pyrazin-2-yl)-3,4-dihydroisoquinolin-1(2H)-one (I-201)

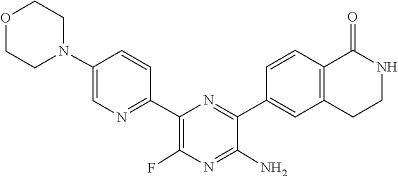 [SMILES: Nc1nc(F)c(-c2ccc(N3CCOCC3)cn2)nc1-c1ccc2c(c1)CCNC2=O]

To a −78° C. solution 4-(6-bromopyridin-3-yl)morpholine (63.5 mg, 0.261 mmol) in anhydrous THF (2 mL) was added n-butyllithium 1.6 M in hexane (0.163 ml, 0.261 mmol). After 30 min, bis(tributyltin) (0.132 ml, 0.261 mmol) was added dropwise and allowed to warm to room temperature overnight. The reaction was quenched with an aqueous saturated ammonium chloride solution, then extracted with EtOAc (3×20 mL). The combined organic layers were washed with brine, dried with anhydrous Na₂SO₄ and concentrated in vacuo to obtain 4-(6-(tributylstannyl)pyridin-3-yl)morpholine intermediate as a yellow oil which was used crude in the next step without further purification. To 4-(6-(tributylstannyl)pyridin-3-yl)morpholine was added 6-(3-amino-5-fluoropyrazin-2-yl)-3,4-dihydroisoquinolin-1(2H)-one, tetrakis(triphenylphosphine)palladium(0) (13.71 mg, 0.012 mmol) and toluene (2 mL). The system was flushed with nitrogen and heated at 110° C. overnight. The reaction was concentrated onto celite and purified by flash chromatography (Biotage, silica gel) eluting with 0-10% MeOH/DCM+1% NH₄OH, then re-purified by flash chromatography (Biotage, reverse phase silica gel) eluting with 0-100% ACN/H₂O. The desired fractions were collected, concentrated and dried on the lyophilizer to afford the title compound (6.1 mg, 12.2% yield) as a pale yellow solid. ¹H NMR (500 MHz, DMSO-d₆) δ ppm 8.36 (d, J=2.57 Hz, 1H), 7.98 (br. s., 1H), 7.94 (d, J=7.95 Hz, 1H), 7.81 (d, J=8.80 Hz, 1H), 7.68 (d, J=8.19 Hz, 1H), 7.64 (s, 1H), 7.43 (dd, J=8.93, 2.81 Hz, 1H), 6.87 (s, 2H), 3.74-3.80 (m, 4H), 3.40-3.44 (m, 2H), 3.21-3.25 (m, 4H), 2.98 (t, J=6.48 Hz, 2H); LCMS: [M+H]⁺=421.39.

### Example 202: 6-(3-amino-6-(3-(azetidin-1-ylmethyl)-4-morpholinophenyl)-5-fluoropyrazin-2-yl)-4-methylisoquinolin-1(2H)-one (I-202)

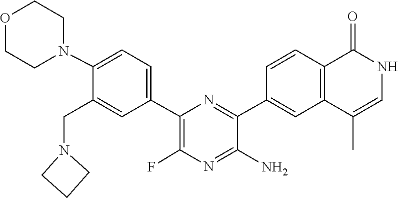 [SMILES: Cc1c[nH]c(=O)c2ccc(-c3nc(-c4ccc(N5CCOCC5)c(CN5CCC5)c4)c(F)nc3N)cc12]

Prepared from 6-(3-amino-6-bromo-5-fluoropyrazin-2-yl)-4-methylisoquinolin-1(2H)-one (40 mg, 0.115 mmol) and 4-(2-(azetidin-1-ylmethyl)-4-(4,4,5,5-tetramethyl-1,3,2-dioxaborolan-2-yl)phenyl)morpholine (49.3 mg, 0.137 mmol) to give the title compound (13.1 mg, 22.8% yield) as a yellow solid. ¹H NMR (500 MHz, DMSO-d₆) b ppm 11.14 (d, J=5.38 Hz, 1H), 8.33 (d, J=8.31 Hz, 1H), 8.00 (s, 1H), 7.89 (s, 1H), 7.85 (d, J=8.31 Hz, 1H), 7.72 (d, J=8.31 Hz, 1H), 7.14 (d, J=8.44 Hz, 1H), 7.07 (d, J=5.38 Hz, 1H), 6.92 (s, 2H), 3.75 (d, J=3.55 Hz, 4H), 3.61 (s, 2H), 3.18 (t, J=6.79 Hz, 4H), 2.93 (br. s., 4H), 2.27 (s, 3H), 1.98 (quin, J=6.85 Hz, 2H); LCMS: [M+H]⁺=501.49.

### Example 203: 6-(3-amino-5-fluoro-6-(4-morpholino-3-(pyrrolidin-1-ylmethyl)phenyl)pyrazin-2-yl)-4-methylisoquinolin-1(2H)-one (I-203)

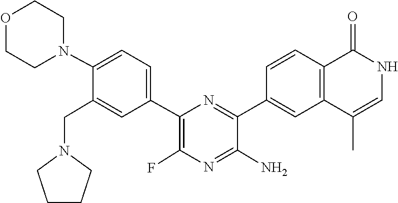 [SMILES: Cc1c[nH]c(=O)c2ccc(-c3nc(-c4ccc(N5CCOCC5)c(CN5CCCC5)c4)c(F)nc3N)cc12]

Prepared from 6-(3-amino-6-bromo-5-fluoropyrazin-2-yl)-4-methylisoquinolin-1(2H)-one (40 mg, 0.115 mmol) and 4-(2-(pyrrolidin-1-ylmethyl)-4-(4,4,5,5-tetramethyl-1,3,2-dioxaborolan-2-yl)phenyl)morpholine (51.2 mg, 0.137 mmol) to give the title compound (15.6 mg, 26.5% yield) as a yellow solid. ¹H NMR (500 MHz, DMSO-d₆) b ppm 11.14 (d, J=5.14 Hz, 1H), 8.32 (d, J=8.19 Hz, 1H), 7.99 (s, 1H), 7.93 (s, 1H), 7.84 (d, J=8.31 Hz, 1H), 7.73 (d, J=8.44 Hz, 1H), 7.15 (d, J=8.44 Hz, 1H), 7.07 (d, J=5.01 Hz, 1H), 6.92 (s, 2H), 3.75 (d, J=4.03 Hz, 4H), 3.66 (s, 2H), 3.30 (s, 4H), 2.97 (br. s., 4H), 2.26 (s, 3H), 1.68 (br. s., 4H); LCMS: [M+H]⁺=515.59.

### Example 204: 6-(3-amino-6-(3-(azetidin-1-ylmethyl)-4-(tetrahydro-2H-pyran-4-yl)phenyl)-5-fluoropyrazin-2-yl)-4-fluoroisoquinolin-1(2H)-one (I-204)

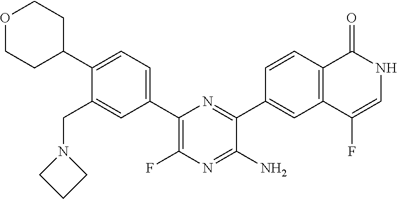 [SMILES: Nc1nc(F)c(-c2ccc(C3CCOCC3)c(CN3CCC3)c2)nc1-c1ccc2c(=O)[nH]cc(F)c2c1]

Prepared from 6-(3-amino-6-bromo-5-fluoropyrazin-2-yl)-4-fluoroisoquinolin-1(2H)-one (40 mg, 0.113 mmol) and 1-(2-(tetrahydro-2H-pyran-4-yl)-5-(4,4,5,5-tetramethyl-1,3,2-dioxaborolan-2-yl)benzyl)azetidine (48.6 mg, 0.136 mmol) to give the title compound (6.0 mg, 10.5% yield) as a tan solid. ¹H NMR (500 MHz, DMSO-d₆) δ ppm 11.21 (br. s., 1H), 8.32 (d, J=7.95 Hz, 1H), 8.07 (s, 1H), 7.96 (d, J=8.07 Hz, 1H), 7.79 (br. s., 1H), 7.72 (d, J=7.70 Hz, 1H), 7.45 (br. s., 1H), 7.37 (d, J=8.07 Hz, 1H), 7.00 (br. s., 2H), 3.96 (d, J=8.80 Hz, 2H), 3.64 (s, 2H), 3.47 (t, J=11.19 Hz, 3H), 3.16 (t, J=6.72 Hz, 4H), 1.98 (t, J=6.79 Hz, 2H), 1.61-1.74 (m, 4H); LCMS: [M+H]⁺=504.44.

### Example 205: 6-(3-amino-5-fluoro-6-(3-(pyrrolidin-1-ylmethyl)-4-(tetrahydro-2H-pyran-4-yl)phenyl)pyrazin-2-yl)-4-methylisoquinolin-1(2H)-one (I-205)

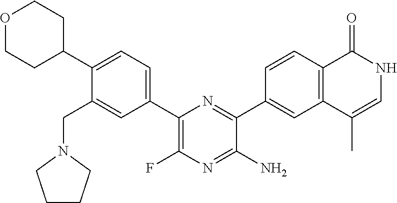 [SMILES: Cc1c[nH]c(=O)c2ccc(-c3nc(-c4ccc(C5CCOCC5)c(CN5CCCC5)c4)c(F)nc3N)cc12]

Prepared from 6-(3-amino-6-bromo-5-fluoropyrazin-2-yl)-4-methylisoquinolin-1(2H)-one (40 mg, 0.115 mmol) and 1-(2-(tetrahydro-2H-pyran-4-yl)-5-(4,4,5,5-tetramethyl-1,3,2-dioxaborolan-2-yl)benzyl)pyrrolidine (51.0 mg, 0.137 mmol) to give the title compound (27.3 mg, 46.4% yield) as a tan solid. ¹H NMR (500 MHz, DMSO-d₆) b ppm 11.15 (br. s., 1H), 8.33 (d, J=8.07 Hz, 1H), 7.99 (br. s., 1H), 7.84 (d, J=8.07 Hz, 1H), 7.81 (br. s., 1H), 7.74 (d, J=7.58 Hz, 1H), 7.38 (d, J=7.70 Hz, 1H), 7.07 (br. s., 1H), 6.96 (br. s., 2H), 3.96 (d, J=8.80 Hz, 2H), 3.68 (br. s., 2H), 3.45 (t, J=11.31 Hz, 2H), 3.20 (br. s., 1H), 2.26 (br. s., 3H), 1.60-1.77 (m, 8H); LCMS: [M+H]⁺=514.46.

### Example 206: 6-(3-amino-6-(3-(azetidin-1-ylmethyl)-4-(tetrahydro-2H-pyran-4-yl)phenyl)-5-fluoropyrazin-2-yl)-4-methylisoquinolin-1(2H)-one (I-206)

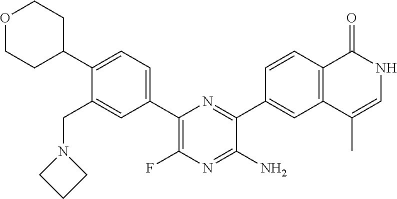 [SMILES: Cc1c[nH]c(=O)c2ccc(-c3nc(-c4ccc(C5CCOCC5)c(CN5CCC5)c4)c(F)nc3N)cc12]

Prepared from 6-(3-amino-6-bromo-5-fluoropyrazin-2-yl)-4-methylisoquinolin-1(2H)-one (40 mg, 0.115 mmol) and 1-(2-(tetrahydro-2H-pyran-4-yl)-5-(4,4,5,5-tetramethyl-1,3,2-dioxaborolan-2-yl)benzyl)azetidine (49.1 mg, 0.137 mmol) to give the title compound (13.4 mg, 23.4% yield) as a grey solid. ¹H NMR (500 MHz, DMSO-d₆) b ppm 11.15 (br. s., 1H), 8.33 (d, J=7.34 Hz, 1H), 7.97-8.04 (m, 1H), 7.85 (d, J=7.34 Hz, 1H), 7.81 (br. s., 1H), 7.72 (d, J=5.99 Hz, 1H), 7.36 (d, J=7.21 Hz, 1H), 7.07 (br. s., 1H), 6.96 (br. s., 2H), 3.96 (d, J=8.68 Hz, 2H), 3.65 (br. s., 2H), 3.44-3.51 (m, 2H), 3.07-3.21 (m, 4H), 2.27 (br. s., 3H), 1.99 (br. s., 2H), 1.55-1.81 (m, 4H); LCMS: [M+H]⁺=500.42.

### Example 207: 6-(3-amino-5-fluoro-6-(3-(pyrrolidin-1-ylmethyl)-4-(tetrahydro-2H-pyran-4-yl)phenyl)pyrazin-2-yl)-4-fluoroisoquinolin-1(2H)-one (I-207)

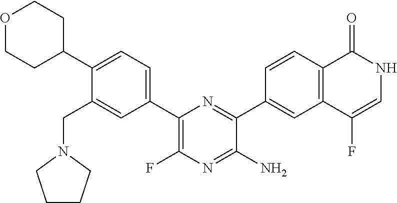 [SMILES: Nc1nc(F)c(-c2ccc(C3CCOCC3)c(CN3CCCC3)c2)nc1-c1ccc2c(=O)[nH]cc(F)c2c1]

Prepared from 6-(3-amino-6-bromo-5-fluoropyrazin-2-yl)-4-fluoroisoquinolin-1(2H)-one (40 mg, 0.113 mmol) and 1-(2-(tetrahydro-2H-pyran-4-yl)-5-(4,4,5,5-tetramethyl-1,3,2-dioxaborolan-2-yl)benzyl)pyrrolidine (50.5 mg, 0.136 mmol) to give the product (9.6 mg, 16.4% yield) as a beige solid. 1H NMR (500 MHz, DMSO-d₆) δ ppm 11.21 (br. s., 1H), 8.32 (d, J=7.58 Hz, 1H), 8.06 (br. s., 1H), 7.95 (d, J=7.82 Hz, 1H), 7.80 (br. s., 1H), 7.74 (d, J=6.97 Hz, 1H), 7.44 (br. s., 1H), 7.39 (d, J=7.70 Hz, 1H), 6.99 (br. s., 2H), 3.96 (d, J=8.68 Hz, 2H), 3.68 (br. s., 2H), 3.40-3.51 (m, 4H), 3.21 (br. s., 2H), 1.55-1.79 (m, 9H); LCMS: [M+H]⁺=518.54.

### Example 208: 6-(3-amino-6-(3-(azetidin-1-ylmethyl)-4-((tetrahydro-2H-pyran-4-yl)oxy)phenyl)-5-fluoropyrazin-2-yl)-3,4-dihydroisoquinolin-1(2H)-one (I-208)

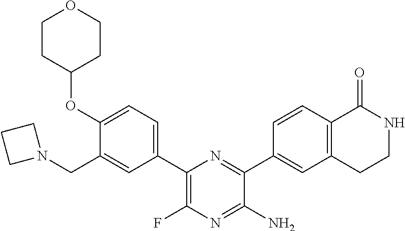 [SMILES: Nc1nc(F)c(-c2ccc(OC3CCOCC3)c(CN3CCC3)c2)nc1-c1ccc2c(c1)CCNC2=O]

### Step 1: Preparation of 1-(5-bromo-2-((tetrahydro-2H-pyran-4-yl)oxy)benzyl)azetidine

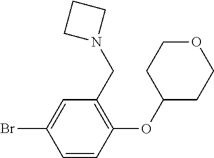 [SMILES: Brc1ccc(OC2CCOCC2)c(CN2CCC2)c1]

Prepared from 5-bromo-2-((tetrahydro-2H-pyran-4-yl)oxy)benzaldehyde (400 mg, 1.403 mmol) azetidine (0.284 ml, 4.21 mmol). The title compound was isolated as a colorless oil (272 mg, 57% yield based on 95% purity). LCMS: [M+H]⁺=326.30

### Step 2: 1-(2-((tetrahydro-2H-pyran-4-yl)oxy)-5-(4,4,5,5-tetramethyl-1,3,2-dioxaborolan-2-yl)benzyl)azetidine

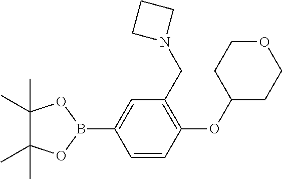 [SMILES: CC1(C)OB(c2ccc(OC3CCOCC3)c(CN3CCC3)c2)OC1(C)C]

Prepared from 1-(5-bromo-2-((tetrahydro-2H-pyran-4-yl)oxy)benzyl)azetidine (180 mg, 0.524 mmol) to give the title compound as a brown solid (196 mg, 95% yield). LCMS: [M+H]⁺=374.50.

### Step 3: 6-(3-amino-6-(3-(azetidin-1-ylmethyl)-4-((tetrahydro-2H-pyran-4-yl)oxy)phenyl)-5-fluoropyrazin-2-yl)-3,4-dihydroisoquinolin-1(2H)-one

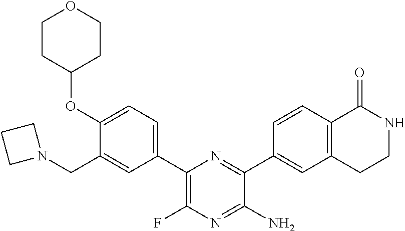 [SMILES: Nc1nc(F)c(-c2ccc(OC3CCOCC3)c(CN3CCC3)c2)nc1-c1ccc2c(c1)CCNC2=O]

Prepared from 6-(3-amino-6-bromo-5-fluoropyrazin-2-yl)-3,4-dihydroisoquinolin-1(2H)-one (46.5 mg, 0.138 mmol) and 1-(2-((tetrahydro-2H-pyran-4-yl)oxy)-5-(4,4,5,5-tetramethyl-1,3,2-dioxaborolan-2-yl)benzyl)azetidine (65 mg, 0.165 mmol) to give the title compound as a beige solid (29 mg, 40% yield). ¹H NMR (500 MHz, DMSO-d₆) δ ppm 8.00 (br. s., 1H), 7.96 (d, J=7.95 Hz, 1H), 7.82 (br. s., 1H), 7.70 (dd, J=13.88, 8.50 Hz, 2H), 7.65 (s, 1H), 7.14 (d, J=8.68 Hz, 1H), 6.81 (br. s., 2H), 4.63-4.79 (m, 1H), 3.85-3.90 (m, 2H), 3.69 (br. s., 2H), 3.52-3.57 (m, 2H), 3.43 (d, J=4.28 Hz, 4H), 3.30-3.32 (m, 2H), 3.30-3.32 (m, 2H), 2.99 (t, J=6.36 Hz, 2H), 1.97-2.10 (m, 4H), 1.66 (td, J=8.31, 4.03 Hz, 2H); LCMS: [M+H]⁺=504.44.

### Example 209: 6-(3-amino-6-(3-(azetidin-1-ylmethyl)-4-((tetrahydro-2H-pyran-4-yl)oxy)phenyl)-5-fluoropyrazin-2-yl)-7-fluoro-3,4-dihydroisoquinolin-1(2H)-one (I-209)

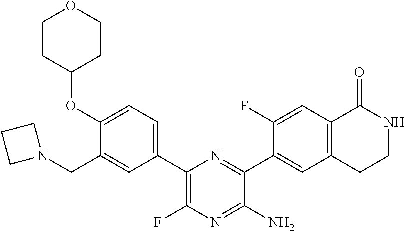 [SMILES: Nc1nc(F)c(-c2ccc(OC3CCOCC3)c(CN3CCC3)c2)nc1-c1cc2c(cc1F)C(=O)NCC2]

Prepared from 6-(3-amino-6-bromo-5-fluoropyrazin-2-yl)-7-fluoro-3,4-dihydroisoquinolin-1(2H)-one (49.0 mg, 0.138 mmol) and 1-(2-((tetrahydro-2H-pyran-4-yl)oxy)-5-(4,4,5,5-tetramethyl-1,3,2-dioxaborolan-2-yl)benzyl)azetidine (65 mg, 0.165 mmol) to give the title compound as a beige solid (40 mg, 53% yield). ¹H NMR (500 MHz, DMSO-d₆) δ ppm 8.16 (br. s., 1H), 7.75-7.81 (m, 1H), 7.76 (br. s., 1H), 7.61-7.70 (m, 2H), 7.52 (d, J=6.72 Hz, 1H), 7.08-7.17 (m, 1H), 6.65-6.86 (m, 2H), 4.66 (dt, J=7.52, 3.82 Hz, 1H), 3.84-3.92 (m, 2H), 3.65 (br. s., 2H), 3.53 (d, J=8.68 Hz, 2H), 3.42 (d, J=4.16 Hz, 4H), 3.23-3.27 (m, 2H), 2.96 (t, J=6.17 Hz, 2H), 1.92-2.08 (m, 4H), 1.60-1.71 (m, 2H), 0.00-0.00 (m, 1H); LCMS: [M+H]+=522.49.

### Example 210: 6-(3-amino-6-(3-(azetidin-1-ylmethyl)-4-((tetrahydro-2H-pyran-4-yl)oxy)phenyl)-5-fluoropyrazin-2-yl)-8-fluoro-3,4-dihydroisoquinolin-1(2H)-one (I-207)

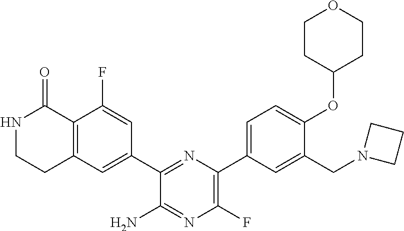 [SMILES: Nc1nc(F)c(-c2ccc(OC3CCOCC3)c(CN3CCC3)c2)nc1-c1cc(F)c2c(c1)CCNC2=O]

Prepared from 6-(3-amino-6-bromo-5-fluoropyrazin-2-yl)-8-fluoro-3,4-dihydroisoquinolin-1(2H)-one (49.0 mg, 0.138 mmol) mmol) and 11-(2-((tetrahydro-2H-pyran-4-yl)oxy)-5-(4,4,5,5-tetramethyl-1,3,2-dioxaborolan-2-yl)benzyl)azetidine (65 mg, 0.165 mmol) to give the title compound as a beige solid (46 mg, 61% yield). ¹H NMR (500 MHz) δ ppm 8.05 (br, DMSO-d₆). s., 1H), 7.81 (br. s., 1H), 7.71 (d, J=8.44 Hz, 1H), 7.50 (s, 1H), 7.43 (d, J=11.98 Hz, 1H), 7.14 (d, J=8.68 Hz, 1H), 6.91 (br. s., 2H), 4.68 (dd, J=7.40, 3.73 Hz, 1H), 3.87 (dd, J=11.07, 4.83 Hz, 2H), 3.66 (br. s., 4H), 3.50-3.58 (m, 4H), 3.24-3.29 (m, 2H), 2.98 (t, J=6.05 Hz, 2H), 1.97-2.08 (m, 4H), 1.66 (ddd, J=12.35, 8.25, 3.85 Hz, 2H); LCMS: [M+H]+=522.61.

### Example 211: (S)-6-(3-amino-6-(3-((dimethylamino)methyl)-4-(2-methylmorpholino)phenyl)-5-fluoropyrazin-2-yl)-3,4-dihydroisoquinolin-1(2H)-one (I-211)

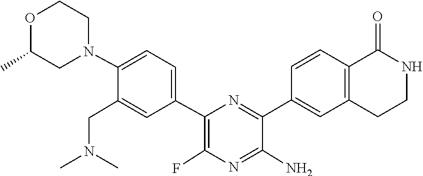 [SMILES: C[C@H]1CN(c2ccc(-c3nc(-c4ccc5c(c4)CCNC5=O)c(N)nc3F)cc2CN(C)C)CCO1]

Prepared from 6-(3-amino-6-bromo-5-fluoropyrazin-2-yl)-3,4-dihydroisoquinolin-1(2H)-one (48.2 mg, 0.143 mmol) and (S)—N,N-dimethyl-1-(2-(2-methylmorpholino)-5-(4,4,5,5-tetramethyl-1,3,2-dioxaborolan-2-yl)phenyl)methanamine (65 mg, 0.171 mmol, prepared in turn from 5-bromo-2-fluorobenzaldehyde and (S)-2-methyl-morpholine in a series of steps analogous to previous examples) to give the title compound as a beige solid (33 mg, 45% yield). ¹H NMR (500 MHz, DMSO-d₆) δ ppm 7.97-8.03 (m, 1H), 7.96 (d, J=7.95 Hz, 1H), 7.87 (s, 1H), 7.74 (d, J=8.19 Hz, 1H), 7.66-7.71 (m, 1H), 7.67 (d, J=8.07 Hz, 1H), 7.64 (s, 1H), 7.14-7.21 (m, 1H), 6.82 (br. s., 2H), 3.87 (d, J=10.64 Hz, 1H), 3.68-3.77 (m, 2H), 3.68-3.77 (m, 2H), 3.47 (br. s., 2H), 3.43 (br. s., 2H), 3.16 (d, J=11.37 Hz, 1H), 3.07 (d, J=11.25 Hz, 1H), 2.99 (t, J=6.30 Hz, 2H), 2.69-2.77 (m, 1H), 2.41-2.46 (m, 1H), 2.20 (br. s., 6H), 1.12 (d, J=6.11 Hz, 3H); LCMS: [M+H]⁺=491.46.

### Example 212: (S)-6-(3-amino-6-(3-((dimethylamino)methyl)-4-(2-methylmorpholino)phenyl)-5-fluoropyrazin-2-yl)-7-fluoro-3,4-dihydroisoquinolin-1(2H)-one (I-212)

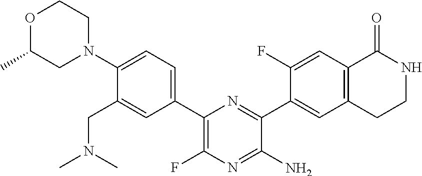 [SMILES: C[C@H]1CN(c2ccc(-c3nc(-c4cc5c(cc4F)C(=O)NCC5)c(N)nc3F)cc2CN(C)C)CCO1]

Prepared from 6-(3-amino-6-bromo-5-fluoropyrazin-2-yl)-7-fluoro-3,4-dihydroisoquinolin-1(2H)-one (50.7 mg, 0.143 mmol) and (S)—N,N-dimethyl-1-(2-(2-methylmorpholino)-5-(4,4,5,5-tetramethyl-1,3,2-dioxaborolan-2-yl)phenyl)methanamine (65 mg, 0.171 mmol) to give the title compound as a beige solid (35 mg, 46% yield). ¹H NMR (500 MHz, DMSO-d₆) δ ppm 8.16 (br. s., 1H), 7.83 (s, 1H), 7.69 (d, J=8.44 Hz, 1H), 7.63-7.67 (m, 1H), 7.63-7.67 (m, 1H), 7.65 (d, J=10.15 Hz, 1H), 7.51 (d, J=6.72 Hz, 1H), 7.14 (d, J=8.44 Hz, 1H), 6.78 (br. s., 2H), 3.82-3.89 (m, 1H), 3.66-3.74 (m, 2H), 3.42-3.47 (m, 4H), 3.14 (d, J=11.25 Hz, 1H), 3.05 (d, J=11.25 Hz, 1H), 2.95 (t, J=6.24 Hz, 2H), 2.71-2.78 (m, 1H), 2.40-2.47 (m, 1H), 2.18 (s, 6H), 1.12 (d, J=6.24 Hz, 3H); LCMS: [M+H]⁺=509.51.

### Example 213: (S)-6-(3-amino-6-(3-((dimethylamino)methyl)-4-(2-methylmorpholino)phenyl)-5-fluoropyrazin-2-yl)-8-fluoro-3,4-dihydroisoquinolin-1(2H)-one (I-213)

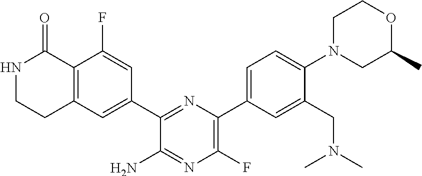 [SMILES: C[C@H]1CN(c2ccc(-c3nc(-c4cc(F)c5c(c4)CCNC5=O)c(N)nc3F)cc2CN(C)C)CCO1]

Prepared from 6-(3-amino-6-bromo-5-fluoropyrazin-2-yl)-8-fluoro-3,4-dihydroisoquinolin-1(2H)-one (50.7 mg, 0.143 mmol) and (S)—N,N-dimethyl-1-(2-(2-methylmorpholino)-5-(4,4,5,5-tetramethyl-1,3,2-dioxaborolan-2-yl)phenyl)methanamine (65 mg, 0.171 mmol) to give the title compound as a beige solid (41 mg, 54% yield). ¹H NMR (500 MHz, DMSO-d₆) δ ppm 8.04 (br. s., 1H), 7.87 (s, 1H), 7.73 (d, J=8.19 Hz, 1H), 7.49 (s, 1H), 7.42 (d, J=11.86 Hz, 1H), 7.16 (d, J=8.44 Hz, 1H), 6.92 (s, 2H), 3.87 (d, J=10.64 Hz, 1H), 3.68-3.77 (m, 2H), 3.46 (br. s., 2H), 3.32-3.33 (m, 2H), 3.32-3.33 (m, 2H), 3.32 (br.s., 2H), 3.17 (d, J=11.00 Hz, 1H), 3.07 (d, J=11.25 Hz, 1H), 2.97 (t, J=6.05 Hz, 2 H), 2.72-2.79 (m, 1H), 2.45 (t, J=10.64 Hz, 1H), 2.20 (s, 6H), 1.08-1.17 (m, 3H); LCMS: [M+H]⁺=509.45.

### Example 214: (S)-6-(3-amino-6-(3-(azetidin-1-ylmethyl)-4-(2-methylmorpholino)phenyl)-5-fluoropyrazin-2-yl)-3,4-dihydroisoquinolin-1(2H)-one (I-214)

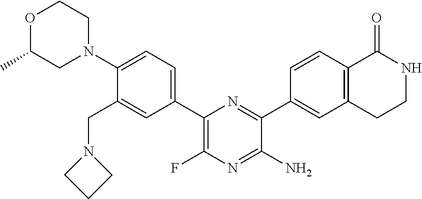 [SMILES: C[C@H]1CN(c2ccc(-c3nc(-c4ccc5c(c4)CCNC5=O)c(N)nc3F)cc2CN2CCC2)CCO1]

Prepared from 6-(3-amino-6-bromo-5-fluoropyrazin-2-yl)-3,4-dihydroisoquinolin-1(2H)-one (46.6 mg, 0.138 mmol) and ((S)-4-(2-(azetidin-1-ylmethyl)-4-(4,4,5,5-tetramethyl-1,3,2-dioxaborolan-2-yl)phenyl)-2-methylmorpholine (65 mg, 0.166 mmol, prepared from a sequence starting with (S)-5-bromo-2-(2-methylmorpholino)benzaldehyde and azetidine analogous to previous examples) to give the title compound as a beige solid (31 mg, 42% yield). ¹H NMR (500 MHz, DMSO-d₆) δ ppm 7.99 (br. s., 1H), 7.96 (d, J=7.95 Hz, 1H), 7.85 (s, 1H), 7.70 (t, J=6.79 Hz, 2H), 7.65 (s, 1H), 7.12 (d, J=8.44 Hz, 1H), 6.83 (s, 2H), 3.87 (d, J=10.76 Hz, 1H), 3.68-3.76 (m, 2H), 3.61 (s, 2H), 3.43 (d, J=4.40 Hz, 2H), 3.18 (t, J=6.36 Hz, 4H), 3.11 (d, J=11.37 Hz, 1H), 3.04 (d, J=11.37 Hz, 1H), 2.99 (t, J=6.36 Hz, 2H), 2.68-2.75 (m, 1H), 2.44 (t, J=10.64 Hz, 1H), 1.99 (quin, J=6.72 Hz, 2H), 1.13 (d, J=6.24 Hz, 3H); LCMS: [M+H]⁺=503.50.

### Example 215: (S)-6-(3-amino-6-(3-(azetidin-1-ylmethyl)-4-(2-methylmorpholino)phenyl)-5-fluoropyrazin-2-yl)-7-fluoro-3,4-dihydroisoquinolin-1(2H)-one (I-215)

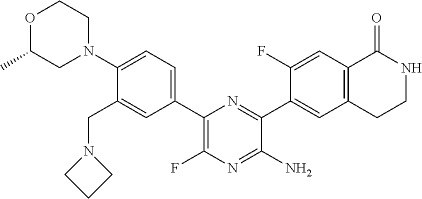 [SMILES: C[C@H]1CN(c2ccc(-c3nc(-c4cc5c(cc4F)C(=O)NCC5)c(N)nc3F)cc2CN2CCC2)CCO1]

Prepared from 6-(3-amino-6-bromo-5-fluoropyrazin-2-yl)-7-fluoro-3,4-dihydroisoquinolin-1(2H)-one (49.1 mg, 0.138 mmol) and (S)-4-(2-(azetidin-1-ylmethyl)-4-(4,4,5,5-tetramethyl-1,3,2-dioxaborolan-2-yl)phenyl)-2-methylmorpholine (65 mg, 0.166 mmol) to give the title compound as a beige solid (22 mg, 29% yield). ¹H NMR (500 MHz, DMSO-d₆) δ ppm 8.16 (br. s., 1H), 7.81 (s, 1H), 7.65 (d, J=9.90 Hz, 2H), 7.52 (d, J=6.72 Hz, 1H), 7.10 (d, J=8.44 Hz, 1H), 6.79 (br. s., 2H), 3.87 (d, J=10.76 Hz, 1H), 3.71 (d, J=9.66 Hz, 2H), 3.60 (s, 2H), 3.43 (br. s., 2H), 3.16 (t, J=6.42 Hz, 4H), 3.10 (d, J=11.37 Hz, 1H), 3.03 (d, J=11.25 Hz, 1H), 2.96 (t, J=6.05 Hz, 2H), 2.71 (t, J=10.33 Hz, 1H), 2.44 (t, J=10.58 Hz, 1H), 1.98 (quin, J=6.69 Hz, 2H), 1.13 (d, J=6.11 Hz, 3H); LCMS: [M+H]⁺=521.48.

### Example 216: (S)-6-(3-amino-6-(3-(azetidin-1-ylmethyl)-4-(2-methylmorpholino)phenyl)-5-fluoropyrazin-2-yl)-8-fluoro-3,4-dihydroisoquinolin-1(2H)-one (I-216)

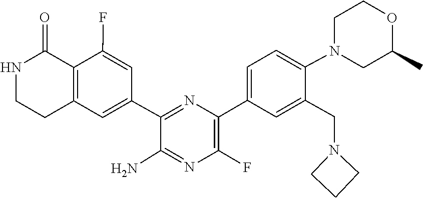 [SMILES: C[C@H]1CN(c2ccc(-c3nc(-c4cc(F)c5c(c4)CCNC5=O)c(N)nc3F)cc2CN2CCC2)CCO1]

Prepared from 6-(3-amino-6-bromo-5-fluoropyrazin-2-yl)-8-fluoro-3,4-dihydroisoquinolin-1(2H)-one (49.1 mg, 0.138 mmol) and (S)-4-(2-(azetidin-1-ylmethyl)-4-(4,4,5,5-tetramethyl-1,3,2-dioxaborolan-2-yl)phenyl)-2-methylmorpholine (65 mg, 0.166 mmol) to give the title compound as a yellow solid (19 mg, 25% yield). ¹H NMR (500 MHz, DMSO-d₆) δ ppm 8.05 (br. s., 1H), 7.85 (s, 1H), 7.70 (d, J=8.19 Hz, 1H), 7.51 (s, 1H), 7.43 (d, J=11.86 Hz, 1H), 7.12 (d, J=8.44 Hz, 1H), 6.84-7.00 (m, 2H), 3.87 (d, J=10.76 Hz, 1H), 3.71 (d, J=9.90 Hz, 2H), 3.61 (br. s., 2H), 3.37 (br. s., 2H), 3.18 (t, J=6.05 Hz, 4H), 3.11 (d, J=11.37 Hz, 1H), 3.04 (d, J=11.37 Hz, 1H), 2.97 (t, J=5.87 Hz, 2H), 2.72 (t, J=10.39 Hz, 1H), 2.41-2.46 (m, 1H), 1.97-2.05 (m, 2H), 1.13 (d, J=6.11 Hz, 3H); LCMS: [M+H]⁺=521.55.

### Example 217: (R)-7-(3-amino-5-fluoro-6-(3-((2-methylpyrrolidin-1-yl)methyl)-4-(tetrahydro-2H-pyran-4-yl)phenyl)pyrazin-2-yl)-5-fluoro-2-methylquinazolin-4(3H)-one (I-217)

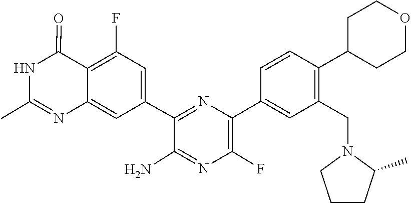

### Example 218: (R)-6-(3-amino-5-fluoro-6-(3-((2-methylpyrrolidin-1-yl)methyl)-4-(tetrahydro-2H-pyran-4-yl)phenyl)pyrazin-2-yl)-8-fluoro-3,4-dihydroisoquinolin-1(2H)-one (I-218)

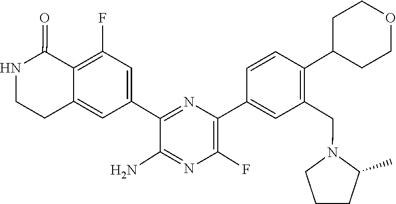 [SMILES: C[C@@H]1CCCN1Cc1cc(-c2nc(-c3cc(F)c4c(c3)CCNC4=O)c(N)nc2F)ccc1C1CCOCC1]

Prepared from 6-(3-amino-6-bromo-5-fluoropyrazin-2-yl)-8-fluoro-3,4-dihydroisoquinolin-1(2H)-one (40 mg, 0.113 mmol) and (R)-2-methyl-1-(2-(tetrahydro-2H-pyran-4-yl)-5-(4,4,5,5-tetramethyl-1,3,2-dioxaborolan-2-yl)benzyl)pyrrolidine hydrochloride (61.8 mg, 0.146 mmol, in turn prepared using a sequence starting from 5-bromo-2-(tetrahydro-2H-pyran-4-yl)benzaldehyde and (R)-2-methylpyrrolidine analogous to earlier Examples) to give the title compound (6.5 mg, 10.6% yield). ¹H NMR (500 MHz, DMSO-d₆) b=8.04 (br s, 1H), 7.77-7.70 (m, 2H), 7.49 (s, 1H), 7.42 (br d, J=12.0 Hz, 1H), 7.38 (d, J=8.7 Hz, 1H), 6.96 (s, 2H), 4.19 (br d, J=12.6 Hz, 1H), 3.97 (br d, J=9.0 Hz, 2H), 3.47-3.25 (m, 5H), 3.08 (br d, J=11.6 Hz, 1H), 2.96 (br t, J=6.2 Hz, 2H), 2.68 (brt, J=7.1 Hz, 1H), 2.42 (br d, J=4.3 Hz, 1H), 2.07 (br d, J=8.3 Hz, 1H), 1.98-1.88 (m, 1H), 1.80-1.49 (m, 6H), 1.38-1.27 (m, 1H), 1.18 (d, J=5.9 Hz, 3H); LCMS: [M+H]⁺=534.52.

### Example 219: (S)-7-(3-amino-5-fluoro-6-(3-((2-methylpyrrolidin-1-yl)methyl)-4-(tetrahydro-2H-pyran-4-yl)phenyl)pyrazin-2-yl)-5-fluoro-2-methylquinazolin-4(3H)-one (I-219)

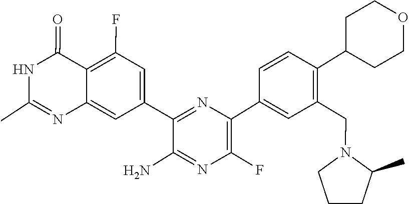

Prepared from 7-(3-amino-6-bromo-5-fluoropyrazin-2-yl)-5-fluoro-2-methylquinazolin-4(3H)-one (40 mg, 0.109 mmol) and (S)-2-methyl-1-(2-(tetrahydro-2H-pyran-4-yl)-5-(4,4,5,5-tetramethyl-1,3,2-dioxaborolan-2-yl)benzyl)pyrrolidine hydrochloride (45.8 mg, 0.109 mmol, in turn in turn prepared using a sequence starting from 5-bromo-2-(tetrahydro-2H-pyran-4-yl)benzaldehyde and (S)-2-methylpyrrolidine analogous to earlier Examples) to give the title compound (20 mg, 33.5% yield). ¹H NMR (500 MHz, DMSO-d₆) b=12.31 (br d, J=2.7 Hz, 1H), 7.81-7.70 (m, 3H), 7.50 (d, J=11.7 Hz, 1H), 7.39 (d, J=8.7 Hz, 1H), 7.02 (s, 2H), 4.20 (d, J=12.6 Hz, 1H), 4.03-3.93 (m, 2H), 3.46-3.37 (m, 2H), 3.33-3.24 (m, 1H), 3.08 (d, J=12.7 Hz, 1H), 2.73-2.64 (m, 1H), 2.46-2.39 (m, 1H), 2.37 (s, 3H), 2.07 (q, J=8.6 Hz, 1H), 2.00-1.89 (m, 1H), 1.82-1.50 (m, 6H), 1.40-1.29 (m, 1H), 1.19 (d, J=5.9 Hz, 3H); LCMS: [M+H]⁺=547.49.

### Example 220: (S)-6-(3-amino-5-fluoro-6-(3-((2-methylpyrrolidin-1-yl)methyl)-4-(tetrahydro-2H-pyran-4-yl)phenyl)pyrazin-2-yl)-8-fluoro-3,4-dihydroisoquinolin-1(2H)-one (I-220)

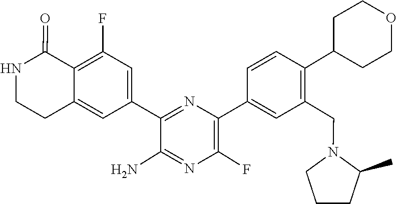 [SMILES: C[C@H]1CCCN1Cc1cc(-c2nc(-c3cc(F)c4c(c3)CCNC4=O)c(N)nc2F)ccc1C1CCOCC1]

Prepared from 6-(3-amino-6-bromo-5-fluoropyrazin-2-yl)-8-fluoro-3,4-dihydroisoquinolin-1(2H)-one (40 mg, 0.113 mmol) and (S)-2-methyl-1-(2-(tetrahydro-2H-pyran-4-yl)-5-(4,4,5,5-tetramethyl-1,3,2-dioxaborolan-2-yl)benzyl)pyrrolidine hydrochloride (47.5 mg, 0.113 mmol) to give the title compound (21 mg, 34.8% yield). ¹H NMR (500 MHz, DMSO-d6) δ=8.04 (br s, 1H), 7.73 (br s, 2H), 7.49 (s, 1H), 7.42 (d, J=11.9 Hz, 1H), 7.38 (d, J=8.7 Hz, 1H), 6.96 (s, 2H), 4.19 (d, J=12.6 Hz, 1H), 3.97 (br d, J=9.3 Hz, 2H), 3.45-3.30 (m, 4H), 3.25 (br t, J=11.3 Hz, 1H), 3.06 (br d, J=12.7 Hz, 1H), 2.96 (br t, J=6.2 Hz, 2H), 2.67 (br t, J=7.5 Hz, 1H), 2.45-2.36 (m, 1H), 2.10-2.00 (m, 1H), 1.98-1.87 (m, 1H), 1.82-1.49 (m, 6H), 1.37-1.27 (m, 1H), 1.17 (d, J=5.9 Hz, 3H); LCMS: [M+H]+=534.52.

### Example 221: 6-(3-amino-5-fluoro-6-(3-methyl-4-(tetrahydro-2H-pyran-4-yl)phenyl)pyrazin-2-yl)-8-fluoro-3,4-dihydroisoquinolin-1(2H)-one (I-221)

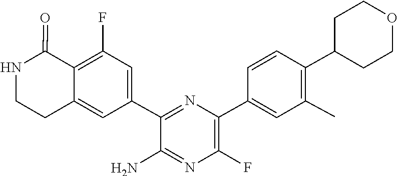 [SMILES: Cc1cc(-c2nc(-c3cc(F)c4c(c3)CCNC4=O)c(N)nc2F)ccc1C1CCOCC1]

Prepared from 6-(3-amino-6-bromo-5-fluoropyrazin-2-yl)-8-fluoro-3,4-dihydroisoquinolin-1(2H)-one (40 mg, 0.113 mmol) reacted with 4,4,5,5-tetramethyl-2-(3-methyl-4-(tetrahydro-2H-pyran-4-yl)phenyl)-1,3,2-dioxaborolane (34.0 mg, 0.113 mmol) to give the title compound (30 mg, 58.6% yield). ¹H NMR (500 MHz, DMSO-d₆) δ=8.04 (br s, 1H), 7.69-7.61 (m, 2H), 7.48 (s, 1H), 7.42 (br d, J=11.9 Hz, 1H), 7.32 (d, J=8.2 Hz, 1H), 6.94 (s, 2H), 3.96 (br dd, J=2.7, 10.8 Hz, 2H), 3.48 (br t, J=11.0 Hz, 2H), 3.36 (br t, J=5.9 Hz, 2H), 3.05-2.93 (m, 3H), 2.38 (s, 3H), 1.76-1.59 (m, 4H); LCMS: [M+H]⁺=451.41.

### Example 222: 6-(3-amino-5-fluoro-6-(3-methyl-4-(tetrahydro-2H-pyran-4-yl)phenyl)pyrazin-2-yl)-7-fluoro-3,4-dihydroisoquinolin-1(2H)-one (I-222)

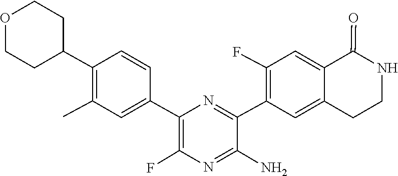 [SMILES: Cc1cc(-c2nc(-c3cc4c(cc3F)C(=O)NCC4)c(N)nc2F)ccc1C1CCOCC1]

Prepared from 6-(3-amino-6-bromo-5-fluoropyrazin-2-yl)-7-fluoro-3,4-dihydroisoquinolin-1(2H)-one (40 mg, 0.113 mmol) and 4,4,5,5-tetramethyl-2-(3-methyl-4-(tetrahydro-2H-pyran-4-yl)phenyl)-1,3,2-dioxaborolane (34.0 mg, 0.113 mmol) to give the title compound (18.3 mg, 35.7% yield). ¹H NMR (500 MHz, DMSO-d₆) δ=8.16 (br s, 1H), 7.70-7.56 (m, 3H), 7.50 (br d, J=6.7 Hz, 1H), 7.30 (d, J=8.1 Hz, 1H), 6.81 (br s, 2H), 3.96 (br d, J=9.8 Hz, 2H), 3.52-3.37 (m, 4H), 3.04-2.90 (m, 3H), 2.36 (s, 3H), 1.75-1.57 (m, 4H); LCMS: [M+H]⁺=451.47.

### Example 223: 6-(3-amino-6-(3-((dimethylamino)methyl)-4-(tetrahydro-2H-pyran-4-yl)phenyl)-5-fluoropyrazin-2-yl)-4,4-difluoro-3,4-dihydroisoquinolin-1(2H)-one (I-223)

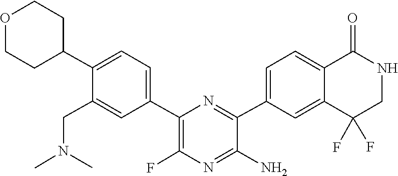 [SMILES: CN(C)Cc1cc(-c2nc(-c3ccc4c(c3)C(F)(F)CNC4=O)c(N)nc2F)ccc1C1CCOCC1]

### Step 1: 6-bromo-4,4-difluoro-3,4-dihydroisoquinolin-1(2H)-one

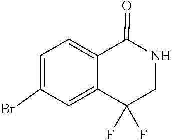 [SMILES: O=C1NCC(F)(F)c2cc(Br)ccc21]

6-bromo-4,4-difluoro-3-hydroxy-3,4-dihydroisoquinolin-1 (2H)-one (2.20 g, 7.91 mmol) was mixed with dichloromethane (80 ml) at r.t. Then methanesulfonic acid (2.054 ml, 31.6 mmol) was added. The reaction mixture turned to a clear solution. Triethylsilane (9.0 mL, 56.6 mmol) was added and the reaction mixture was stirred at r.t. until the reaction was done. The reaction mixture was washed with sat-NaHCO₃ and brine, dried with MgSO₄ and filtered. The filtrate was concentrated with silica gel, purified by column chromatography, eluted with 20-40% EtOAc in hexanes to give the product. LCMS: [M+H]⁺=261.92.

### Step 2: 4,4-difluoro-6-(4,4,5,5-tetramethyl-1,3,2-dioxaborolan-2-yl)-3,4-dihydroisoquinolin-1(2H)-one

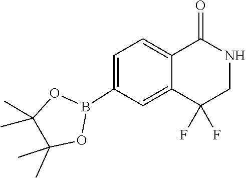 [SMILES: CC1(C)OB(c2ccc3c(c2)C(F)(F)CNC3=O)OC1(C)C]

4,4-difluoro-6-(4,4,5,5-tetramethyl-1,3,2-dioxaborolan-2-yl)-3,4-dihydroisoquinolin-1(2H)-one (1.26 g, 65.1% yield) (˜67% pure) from (1.64 g, 6.26 mmol) was mixed with bis(pinacolato)diboron (1.907 g, 7.51 mmol), potassium acetate (1.843 g, 18.77 mmol) and [1,1′-bis(diphenylphosphino)ferrocene]dichloropalladium(II) (0.459 g, 0.626 mmol) in 1,4-dioxane (38 ml) at 90° for 1.5 hours to give the product (1.26 g, 65.1% yield). LCMS: [M+H]⁺=310.00;

### Step 3: 6-(3-amino-6-bromo-5-fluoropyrazin-2-yl)-4,4-difluoro-3,4-dihydroisoquinolin-1(2H)-one

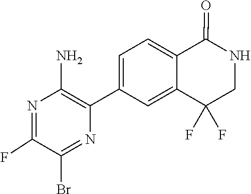 [SMILES: Nc1nc(F)c(Br)nc1-c1ccc2c(c1)C(F)(F)CNC2=O]

Prepared from reacting 4-difluoro-6-(4,4,5,5-tetramethyl-1,3,2-dioxaborolan-2-yl)-3,4-dihydroisoquinolin-1(2H)-one (1.26 g, 4.08 mmol)(67% pure), 5-bromo-6-fluoro-3-iodopyrazin-2-amine (1.296 g, 4.08 mmol), Na₂CO₃H₂O (1.011 g, 8.15 mmol) and bis(triphenylphosphine)palladium(II) dichloride (0.286 g, 0.408 mmol) in acetonitrile (40 ml) at 88° C. overnight. Standard workup to give the product (657 mg, 43.1% yield). ¹H NMR (500 MHz, DMSO-d₆) δ=8.61-8.60 (m, 1H), 8.54 (br s, 1H), 8.07 (d, J=8.1 Hz, 1H), 7.99 (d, J=8.2 Hz, 1H), 7.91 (s, 1H), 7.11 (s, 2H), 4.01-3.90 (m, 2H); LCMS: [M+H]⁺=373.19.

### Step 3: 6-(3-amino-6-(3-((dimethylamino)methyl)-4-(tetrahydro-2H-pyran-4-yl)phenyl)-5-fluoropyrazin-2-yl)-4,4-difluoro-3,4-dihydroisoquinolin-1(2H)-one

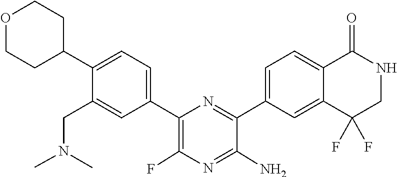 [SMILES: CN(C)Cc1cc(-c2nc(-c3ccc4c(c3)C(F)(F)CNC4=O)c(N)nc2F)ccc1C1CCOCC1]

Prepared from 6-(3-amino-6-bromo-5-fluoropyrazin-2-yl)-4,4-difluoro-3,4-dihydroisoquinolin-1(2H)-one (40 mg, 0.107 mmol) and N,N-dimethyl-1-(2-(tetrahydro-2H-pyran-4-yl)-5-(4,4,5,5-tetramethyl-1,3,2-dioxaborolan-2-yl)phenyl)methanamine hydrochloride (40.9 mg, 0.107 mmol) to give the title compound (23.4 mg, 42.1% yield). ¹H NMR (500 MHz, METHANOL-d₄) δ=8.23-8.18 (m, 1H), 8.17-8.12 (m, 2H), 7.89-7.83 (m, 2H), 7.42 (d, J=8.1 Hz, 1H), 4.06 (br dd, J=3.9, 11.1 Hz, 2H), 3.98 (t, J=12.7 Hz, 2H), 3.66-3.54 (m, 4H), 3.30-3.24 (m, 1H), 2.29 (s, 6H), 1.86 (dq, J=3.4, 12.5 Hz, 2H), 1.73 (br d, J=12.3 Hz, 2H); LCMS: [M+H]⁺=512.39.

### Example 224: 6-(3-amino-6-(3-((ethyl(methyl)amino)methyl)-4-(tetrahydro-2H-pyran-4-yl)phenyl)-5-fluoropyrazin-2-yl)-4,4-difluoro-3,4-dihydroisoquinolin-1(2H)-one (I-224)

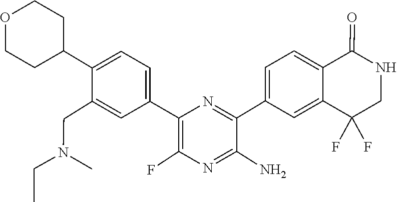 [SMILES: CCN(C)Cc1cc(-c2nc(-c3ccc4c(c3)C(F)(F)CNC4=O)c(N)nc2F)ccc1C1CCOCC1]

Prepared from 6-(3-amino-6-bromo-5-fluoropyrazin-2-yl)-4,4-difluoro-3,4-dihydroisoquinolin-1(2H)-one (30 mg, 0.08 mmol) and N-methyl-N-(2-(tetrahydro-2H-pyran-4-yl)-5-(4,4,5,5-tetramethyl-1,3,2-dioxaborolan-2-yl)benzyl)ethanamine (28.9 mg, 0.080 mmol) to give the title compound (25.4 mg, 58.5% yield). ¹H NMR (500 MHz, METHANOL-d₄) δ=8.22-8.18 (m, 1H), 8.17-8.12 (m, 2H), 7.89-7.82 (m, 2H), 7.42 (d, J=8.7 Hz, 1H), 4.06 (br dd, J=3.9, 11.4 Hz, 2H), 3.97 (t, J=12.7 Hz, 2H), 3.64 (br s, 2H), 3.59 (br t, J=11.6 Hz, 2H), 3.39-3.31 (m, 1H), 2.62-2.46 (m, 2H), 2.24 (s, 3H), 1.95-1.79 (m, 2H), 1.73 (br d, J=11.9 Hz, 2H), 1.16 (t, J=7.1 Hz, 3H); LCMS: [M+H]⁺=526.50.

### Example 225 6-(3-amino-6-(4-(3,6-dihydro-2H-pyran-4-yl)-3-((ethyl(methyl)amino)methyl)phenyl)-5-fluoropyrazin-2-yl)-4,4-difluoro-3,4-dihydroisoquinolin-1(2H)-one (I-225)

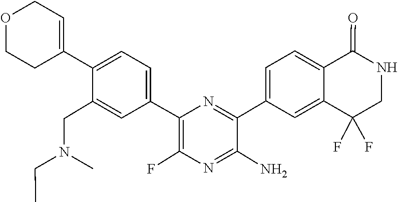 [SMILES: CCN(C)Cc1cc(-c2nc(-c3ccc4c(c3)C(F)(F)CNC4=O)c(N)nc2F)ccc1C1=CCOCC1]

Prepared from 6-(3-amino-6-bromo-5-fluoropyrazin-2-yl)-4,4-difluoro-3,4-dihydroisoquinolin-1(2H)-one (40 mg, 0.107 mmol) reacted with N-(2-(3,6-dihydro-2H-pyran-4-yl)-5-(4,4,5,5-tetramethyl-1,3,2-dioxaborolan-2-yl)benzyl)-N-methylethanamine hydrochloride (42.2 mg, 0.107 mmol) to give the title compound (25 mg, 41.3% yield). 1H NMR (500 MHz, METHANOL-d4) δ=8.22-8.18 (m, 1H), 8.18-8.13 (m, 2H), 8.06 (s, 1H), 7.82 (br d, J=8.1 Hz, 1H), 7.23 (d, J=7.9 Hz, 1H), 5.69 (br s, 1H), 4.28 (br d, J=2.2 Hz, 2H), 4.03-3.96 (m, 2H), 3.96-3.89 (m, 3H), 3.64 (s, 2H), 2.50 (q, J=7.1 Hz, 2H), 2.39 (br s, 2H), 2.23 (s, 3H), 1.11 (t, J=7.1 Hz, 3H); LCMS: [M+H]⁺=524.49.

### Example 226: (R)-6-(3-amino-5-fluoro-6-(3-((2-tmethylpyrrolidin-1-yl)methyl)-4-(tetrahydro-2H-pyran-4-yl)phenyl)pyrazin-2-yl)-4-fluoroisoquinolin-1(2H)-one (I-226)

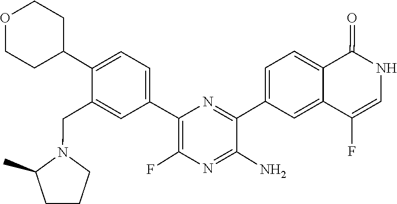 [SMILES: C[C@@H]1CCCN1Cc1cc(-c2nc(-c3ccc4c(=O)[nH]cc(F)c4c3)c(N)nc2F)ccc1C1CCOCC1]

Prepared from a mixture of 6-(3-amino-6-bromo-5-fluoropyrazin-2-yl)-4-fluoroisoquinolin-1(2H)-one and 6-(3-amino-6-bromo-5-fluoropyrazin-2-yl)-4-chloroisoquinolin-1(2H)-one (4:1) (60 mg, 0.170 mmol), and (R)-2-methyl-1-(2-(tetrahydro-2H-pyran-4-yl)-5-(4,4,5,5-tetramethyl-1,3,2-dioxaborolan-2-yl)benzyl)pyrrolidine (65.5 mg, 0.170 mmol) to give after purification the title compound (19.6 mg, 21.50% yield) as a yellow solid. ¹H NMR (500 MHz, METHANOL-d₄) δ=8.47 (d, J=8.3 Hz, 1H), 8.26 (s, 1H), 8.08 (d, J=8.4 Hz, 1H), 7.93-7.85 (m, 2H), 7.43 (d, J=8.1 Hz, 1H), 7.30 (d, J=5.5 Hz, 1H), 4.31 (br d, J=13.0 Hz, 1H), 4.13-4.03 (m, 2H), 3.60 (br t, J=11.7 Hz, 2H), 3.48-3.36 (m, 2H), 3.00-2.85 (m, 1H), 2.65-2.45 (m, 1H), 2.33-2.18 (m, 1H), 2.11-1.99 (m, 1H), 1.98-1.62 (m, 6H), 1.55-1.43 (m, 1H), 1.30 (br d, J=6.0 Hz, 3H); LCMS: [M+H]⁺=532.39.

### Example 227: (R)-6-(3-amino-5-fluoro-6-(3-((2-methylpyrrolidin-1-yl)methyl)-4-(tetrahydro-2H-pyran-4-yl)phenyl)pyrazin-2-yl)-4-chloroisoquinolin-1(2H)-one (I-227)

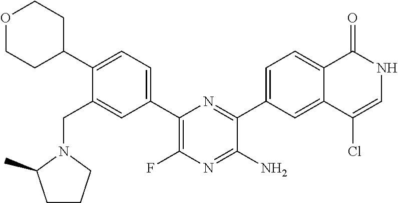 [SMILES: C[C@@H]1CCCN1Cc1cc(-c2nc(-c3ccc4c(=O)[nH]cc(Cl)c4c3)c(N)nc2F)ccc1C1CCOCC1]

The above purification also provided (3.5 mg, 3.67% yield) of (R)-6-(3-amino-5-fluoro-6-(3-((2-methylpyrrolidin-1-yl)methyl)-4-(tetrahydro-2H-pyran-4-yl)phenyl)pyrazin-2-yl)-4-chloroisoquinolin-1(2H)-one. ¹H NMR (500 MHz, METHANOL-d₄) δ=8.49 (d, J=8.3 Hz, 1H), 8.38 (s, 1H), 8.06 (br d, J=8.4 Hz, 1H), 7.93 (br s, 1H), 7.90 (br d, J=7.9 Hz, 1H), 7.47-7.41 (m, 2H), 4.34 (br d, J=13.0 Hz, 1H), 4.16-4.03 (m, 2H), 3.61 (br t, J=11.6 Hz, 2H), 3.33 (br d, J=1.1 Hz, 2H), 3.06-2.83 (m, 1H), 2.75-2.57 (m, 1H), 2.46-2.26 (m, 1H), 2.15-2.01 (m, 1H), 1.98-1.66 (m, 6H), 1.57-1.45 (m, 1H), 1.31 (br d, J=5.5 Hz, 3H); LCMS: [M+H]⁺=548.37.

### Example 228: (S)-6-(3-amino-5-fluoro-6-(3-((2-methylpyrrolidin-1-yl)methyl)-4-(tetrahydro-2H-pyran-4-yl)phenyl)pyrazin-2-yl)-4-fluoroisoquinolin-1(2H)-one (I-228)

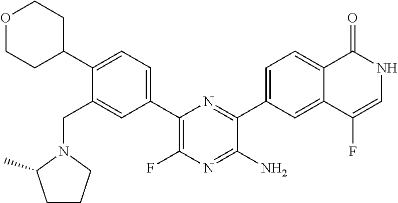 [SMILES: C[C@H]1CCCN1Cc1cc(-c2nc(-c3ccc4c(=O)[nH]cc(F)c4c3)c(N)nc2F)ccc1C1CCOCC1]

Prepared from a mixture of 6-(3-amino-6-bromo-5-fluoropyrazin-2-yl)-4-fluoroisoquinolin-1(2H)-one and 6-(3-amino-6-bromo-5-fluoropyrazin-2-yl)-4-chloroisoquinolin-1(2H)-one (4:1) (60 mg, 0.170 mmol) and (S)-2-methyl-1-(2-(tetrahydro-2H-pyran-4-yl)-5-(4,4,5,5-tetramethyl-1,3,2-dioxaborolan-2-yl)benzyl)pyrrolidine (65.5 mg, 0.170 mmol) to give the title compound (13.2 mg, 14.5% yield). ¹H NMR (500 MHz, METHANOL-d₄+drop of CF₃CO₂D) δ=8.46 (d, J=8.4 Hz, 1H), 8.22 (s, 1H), 8.12-8.00 (m, 3H), 7.58 (d, J=8.3 Hz, 1H), 7.27 (d, J=5.5 Hz, 1H), 4.74 (d, J=13.7 Hz, 1H), 4.37 (d, J=13.7 Hz, 1H), 4.12-4.03 (m, 2H), 3.75-3.60 (m, 3H), 3.55-3.45 (m, 1H), 3.16-3.05 (m, 1H), 2.48-2.34 (m, 1H), 2.19-2.08 (m, 1H), 2.06-1.84 (m, 4H), 1.83-1.70 (m, 3H), 1.49 (d, J=6.5 Hz, 3H); LCMS: [M+H]⁺=532.51.

### Example 229: (S)-6-(3-amino-5-fluoro-6-(3-((2-methylpyrrolidin-1-yl)methyl)-4-(tetrahydro-2H-pyran-4-yl)phenyl)pyrazin-2-yl)-4-chloroisoquinolin-1(2H)-one (I-229)

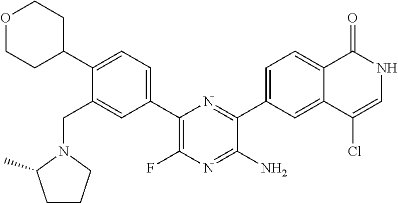 [SMILES: C[C@H]1CCCN1Cc1cc(-c2nc(-c3ccc4c(=O)[nH]cc(Cl)c4c3)c(N)nc2F)ccc1C1CCOCC1]

The reaction from the previous example afforded during the purification (3.1 mg. 3.0% yield) of the title compound. ¹H NMR (500 MHz, METHANOL-d₄) δ=8.38 (d, J=8.4 Hz, 1H), 8.27 (s, 1H), 7.95 (br d, J=8.1 Hz, 1H), 7.86-7.76 (m, 2H), 7.37-7.30 (m, 2H), 4.31-4.21 (m, 1H), 4.04-3.93 (m, 2H), 3.35-3.62 (m, 4H), 2.98-2.79 (m, 1H), 2.65-2.45 (m, 1H), 2.37-2.18 (m, 1H), 2.09-1.92 (m, 1H), 1.90-1.52 (m, 6H), 1.45-1.34 (m, 1H), 1.29-1.16 (m, 3H); LCMS: [M+H]⁺=548.16.

### Example 230: 6-(3-amino-6-(3-((dimethylamino)methyl)-4-((2R,6S)-2,6-dimethylmorpholino)phenyl)-5-fluoropyrazin-2-yl)-4-fluoroisoquinolin-1(2H)-one (I-230)

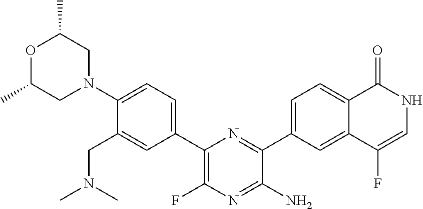 [SMILES: C[C@@H]1CN(c2ccc(-c3nc(-c4ccc5c(=O)[nH]cc(F)c5c4)c(N)nc3F)cc2CN(C)C)C[C@H](C)O1]

Prepared from a mixture of 6-(3-amino-6-bromo-5-fluoropyrazin-2-yl)-4-fluoroisoquinolin-1(2H)-one and 6-(3-amino-6-bromo-5-fluoropyrazin-2-yl)-4-chloroisoquinolin-1(2H)-one (4:1) (60 mg, 0.170 mmol) and 1-(2-(cis-2,6-dimethylmorpholino)-5-(4,4,5,5-tetramethyl-1,3,2-dioxaborolan-2-yl)phenyl)-N,N-dimethylmethanamine hydrochloride (69.8 mg, 0.170 mmol) to give after purification the title compound (27.9 mg, 31.1% yield)¹H NMR (500 MHz, METHANOL-d₄) δ=8.45 (d, J=8.4 Hz, 1H), 8.23 (s, 1H), 8.06 (d, J=8.3 Hz, 1H), 8.02 (s, 1H), 7.88 (br d, J=8.4 Hz, 1H), 7.28 (d, J=5.4 Hz, 1H), 7.23 (d, J=8.4 Hz, 1H), 3.95-3.85 (m, 2H), 3.71 (s, 2H), 3.04 (br d, J=11.4 Hz, 2H), 2.48 (t, J=10.8 Hz, 2H), 2.34 (s, 6H), 1.20 (d, J=6.1 Hz, 6H); LCMS: [M+H]⁺=521.15

### Example 231: 6-(3-amino-6-(3-((dimethylamino)methyl)-4-((2R,6S)-2,6-dimethylmorpholino)phenyl)-5-fluoropyrazin-2-yl)-4-chloroisoquinolin-1(2H)-one (I-231)

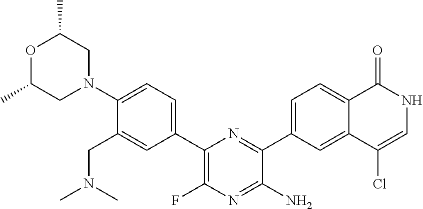 [SMILES: C[C@@H]1CN(c2ccc(-c3nc(-c4ccc5c(=O)[nH]cc(Cl)c5c4)c(N)nc3F)cc2CN(C)C)C[C@H](C)O1]

The purification from the proceeding Example also afforded the title compound (7.3 mg. 7.35% yield). ¹H NMR (500 MHz, METHANOL-d₄) δ=8.47 (d, J=8.3 Hz, 1H), 8.35 (s, 1H), 8.08-8.00 (m, 2H), 7.88 (br d, J=8.7 Hz, 1H), 7.43 (s, 1H), 7.24 (d, J=8.4 Hz, 1H), 3.94-3.85 (m, 2H), 3.73 (br s, 2H), 3.05 (br d, J=11.1 Hz, 2H), 2.49 (br t, J=10.9 Hz, 2H), 2.35 (s, 6H), 1.20 (d, J=6.2 Hz, 6H); [M+H]⁺=537.25.

### Example 232: (R)-6-(3-amino-5-fluoro-6-(3-((2-methylpyrrolidin-1-yl)methyl)-4-(tetrahydro-2H-pyran-4-yl)phenyl)pyrazin-2-yl)-4,4-difluoro-3,4-dihydroisoquinolin-1(2H)-one (I-232)

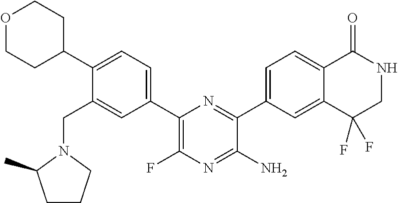 [SMILES: C[C@@H]1CCCN1Cc1cc(-c2nc(-c3ccc4c(c3)C(F)(F)CNC4=O)c(N)nc2F)ccc1C1CCOCC1]

Prepared from 6-(3-amino-6-bromo-5-fluoropyrazin-2-yl)-4,4-difluoro-3,4-dihydroisoquinolin-1 (2H)-one (30 mg, 0.08 mmol) reacted with (R)-2-methyl-1-(2-(tetrahydro-2H-pyran-4-yl)-5-(4,4,5,5-tetramethyl-1,3,2-dioxaborolan-2-yl)benzyl)pyrrolidine hydrochloride (33.9 mg, 0.080 mmol) to give the title compound (18.6 mg, 36.2% yield). ¹H NMR (500 MHz, METHANOL-d₄) δ=8.23-8.11 (m, 3H), 7.93-7.84 (m, 2H), 7.43 (d, J=8.2 Hz, 1H), 4.35 (br d, J=12.8 Hz, 1H), 4.06 (brdd, J=4.9, 11.2 Hz, 2H), 3.98 (t, J=12.8 Hz, 2H), 3.59 (br t, J=11.9 Hz, 2H), 3.42-3.33 (m, 2H), 2.98 (br s, 1H), 2.81-2.63 (m, 1H), 2.48-2.32 (m, 1H), 2.14-2.02 (m, 1H), 1.91-1.67 (m, 6H), 1.55-1.44 (m, 1H), 1.35-1.27 (m, 3H); LCMS: [M+H]⁺=552.44.

### Example 233: (S)-6-(3-amino-5-fluoro-6-(3-((2-methylpyrrolidin-1-yl)methyl)-4-(tetrahydro-2H-pyran-4-yl)phenyl)pyrazin-2-yl)-4,4-difluoro-3,4-dihydroisoquinolin-1(2H)-one (I-233)

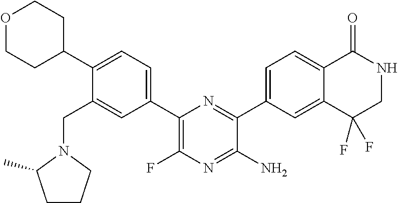 [SMILES: C[C@H]1CCCN1Cc1cc(-c2nc(-c3ccc4c(c3)C(F)(F)CNC4=O)c(N)nc2F)ccc1C1CCOCC1]

Prepared from 6-(3-amino-6-bromo-5-fluoropyrazin-2-yl)-4,4-difluoro-3,4-dihydroisoquinolin-1(2H)-one (30 mg, 0.08 mmol) reacted with (S)-2-methyl-1-(2-(tetrahydro-2H-pyran-4-yl)-5-(4,4,5,5-tetramethyl-1,3,2-dioxaborolan-2-yl)benzyl)pyrrolidine hydrochloride (33.9 mg, 0.080 mmol), potassium carbonate (44.4 mg, 0.322 mmol) and tetrakis(triphenylphosphine)Palladium(0) (9.29 mg, 8.04 μmol) in 1,4-dioxane (2 ml) and water (0.5 ml) in MW at 120° C. for 50 mins to give the title compound (7.6 mg, 16.8% yield). ¹H NMR (500 MHz, METHANOL-d₄) δ=8.29-8.11 (m, 3H), 7.95-7.80 (m, 2H), 7.41 (br d, J=8.1 Hz, 1H), 4.30 (br d, J=10.5 Hz, 1H), 4.13-4.03 (m, 2H), 3.98 (t, J=12.8 Hz, 2H), 3.58 (br t, J=11.7 Hz, 2H), 3.43-3.34 (m, 2H), 2.91 (br d, J=5.6 Hz, 1H), 2.70-2.46 (m, 1H), 2.36-2.15 (m, 1H), 2.13-1.99 (m, 1H), 1.96-1.61 (m, 6H), 1.56-1.39 (m, 1H), 1.28 (br d, J=5.6 Hz, 3H); LCMS: [M+H]⁺=552.42.

### Example 234: 6-(3-amino-5-fluoro-6-(4-(4-isopropylpiperazin-1-yl)phenyl)pyrazin-2-yl)-7-fluoro-4-methylisoquinolin-1(2H)-one (I-234)

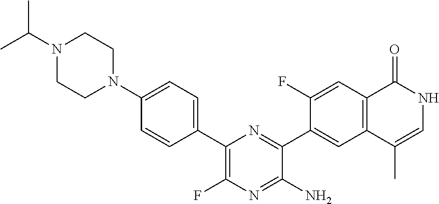 [SMILES: Cc1c[nH]c(=O)c2cc(F)c(-c3nc(-c4ccc(N5CCN(C(C)C)CC5)cc4)c(F)nc3N)cc12]

### Step 1: (E)-3-(3-bromo-4-fluorophenyl)but-2-enoic acid

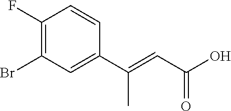

To THF (75 mL) at 0° C. under nitrogen atmosphere, sodium hydride (60%, 1.38 g, 34.56 mmol) was added portion wise. Triethyl phosphonoacetate (6.85 mL, 34.56 mmol) was added dropwise and stirred at 0° C. for 20 min and the resulting mixture was added dropwise to a solution of 1-(3-bromo-4-fluorophenyl)ethan-1-one (5 g, 23.04 mmol) in THF (50 mL) and the resulting mixture was refluxed for 16 h. The mixture was then diluted with water (200 mL) and extracted with DCM (3×150 mL). The organic layer was dried over Na₂SO₄ and concentrated under vacuum to afford a yellow oil. The oil was dissolved in MeOH (25 mL) and NaH (4.6 g, 226.13 mmol) and water (15 mL) were added and the reaction mass was heated at 50° C. for 2 h. The reaction mass was concentrated to evaporate the organics and the resulting aqueous solution was acidified by aqueous 2M HCl and extracted with EtOAc (2×150 mL). The combined organic layers were dried over Na₂SO₄ and concentrated under vacuum to afford the product (3.5 g, 58.6% yield) as white solid. LCMS: [M+H]⁺=258.9.

### Step 2: (E)-3-(3-bromo-4-fluorophenyl)but-2-enoyl chloride

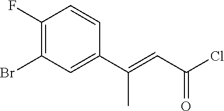

To a cooled solution of (E)-3-(3-bromo-4-fluorophenyl)but-2-enoic acid (3.5 g, 13.58 mmol) in DCM (35 mL) and DMF (0.2 mL) at 0° C., oxalyl chloride (2.05 g, 16.21 mmol) was added dropwise and the reaction mass was allowed to warm to RT and stirred at same temperature for 3 h. After completion the reaction, reaction mass was concentrated and azeotrope with toluene (2×20 mL) and DCM (2×20 mL) to give the crude product (3.5 g) as white solid which was used as such in the next step without analysis and further purification.

### Step 3: (E)-3-(3-bromo-4-fluorophenyl)but-2-enoyl azide

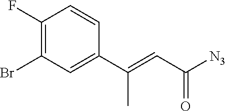 [SMILES: C/C(=C\C(=O)N=[N+]=[N-])c1ccc(F)c(Br)c1]

To a cooled solution of crude of (E)-3-(3-bromo-4-fluorophenyl)but-2-enoyl chloride (3.5 g, 12.61 mmol) in 1,4 dioxane (35 mL) at 0° C., a suspension of sodium azide (1.47 g, 22.70 mmol) in 1:1 mixture of 1,4-dioxane and water (15 mL) was added and the reaction mas was gradually warm to room temperature and stirred at same temperature for a 1.5 h. After completion reaction, reaction mass was diluted with water (30 mL) and extracted with diethyl ether (2×100 mL). The combined organic layers were back washed with saturated aq. NaHCO₃ (3×100 mL) and water (3×100 mL) and dried over Na₂SO₄ and organic layer was directly used for next step.

### Step 4: 6-bromo-7-fluoro-4-methylisoquinolin-1(2H)-one

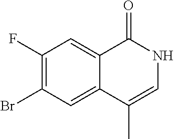

The ether layer of (E)-3-(3-bromo-4-fluorophenyl)but-2-enoyl azide was treated with 1,2 dichlorobenzene (30 mL) and the ether was removed under vacuum to give a solution of (E)-3-(3-bromo-4-fluorophenyl)but-2-enoyl azide in 1,2 dichlorobenzene. The acyl azide solution in 1,2 dichlorobenzene was added dropwise over 30 min to a solution of iodine (0.4 g) in 1,2 dichlorobenzene (30 mL) at 120° C. The mixture was then stirred at 190° C. for 16 h, allowed to cool room temperature and added to hexane (1000 mL). The suspension was stirred for 1 h and the resulting solid was washed with EtOAc (50 mL) and DCM (50 mL) and dried under vacuum to give the product (0.8 g, 25.36%) as pale-yellow solid. LCMS: [M+2]⁺=258.

### Step 4: (7-fluoro-4-methyl-1-oxo-1,2-dihydroisoquinolin-6-yl)boronic acid

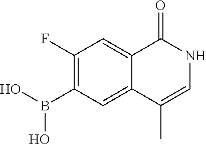 [SMILES: Cc1c[nH]c(=O)c2cc(F)c(B(O)O)cc12]

Prepared from 6-bromo-7-fluoro-4-methylisoquinolin-1(2H)-one (800 mg, 3.12 mmol) to give the boronic acid which was used in the next step without further purification. LCMS: [M+H]+=222.29.

### Step 5: 6-(3-amino-6-bromo-5-fluoropyrazin-2-yl)-7-fluoro-4-methylisoquinolin-1(2H)-one

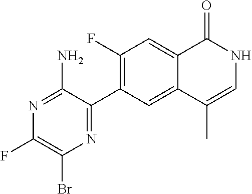 [SMILES: Cc1c[nH]c(=O)c2cc(F)c(-c3nc(Br)c(F)nc3N)cc12]

Prepared from 5-bromo-6-fluoro-3-iodopyrazin-2-amine (478 mg, 1.504 mmol) and (7-fluoro-4-methyl-1-oxo-1,2-dihydroisoquinolin-6-yl)boronic acid (432 mg, 1.955 mmol) to give the product (95 mg, 17.2% yield) as a beige solid. LCMS: [M+H]⁺=367.23.

### Step 6: 6-(3-amino-5-fluoro-6-(4-(4-isopropylpiperazin-1-yl)phenyl)pyrazin-2-yl)-7-fluoro-4-methylisoquinolin-1(2H)-one

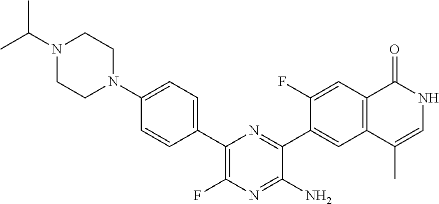 [SMILES: Cc1c[nH]c(=O)c2cc(F)c(-c3nc(-c4ccc(N5CCN(C(C)C)CC5)cc4)c(F)nc3N)cc12]

Prepared from 6-(3-amino-6-bromo-5-fluoropyrazin-2-yl)-7-fluoro-4-methylisoquinolin-1(2H)-one (30 mg, 0.057 mmol) and 4-(4-isopropylpiperazinyl)phenylboronic acid, pinacol ester (22.67 mg, 0.069 mmol) to give the title compound (2.7 mg, 62% yield) as a tan solid. ¹H NMR (500 MHz, DMSO-d₆) δ ppm 11.26 (br. s., 1H), 7.99 (d, J=10.27 Hz, 1H), 7.82 (d, J=6.60 Hz, 1H), 7.70 (d, J=8.19 Hz, 2H), 7.05 (br. s., 1H), 6.99 (d, J=8.80 Hz, 2H), 6.72 (br. s., 2H), 3.17 (br. s., 4H), 2.65-2.70 (m, 1H), 2.56 (d, J=4.28 Hz, 4H), 2.23 (s, 3H), 1.00 (d, J=6.36 Hz, 6H); LCMS: [M+H]⁺=491.46.

### Example 235: 6-(3-amino-6-(3-((dimethylamino)methyl)-4-(tetrahydro-2H-pyran-4-yl)phenyl)-5-fluoropyrazin-2-yl)-7-fluoro-4-methylisoquinolin-1(2H)-one (I-235)

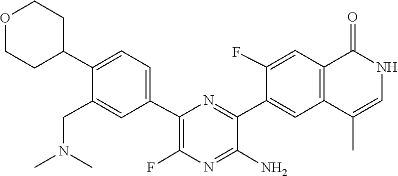 [SMILES: Cc1c[nH]c(=O)c2cc(F)c(-c3nc(-c4ccc(C5CCOCC5)c(CN(C)C)c4)c(F)nc3N)cc12]

Prepared from 6-(3-amino-6-bromo-5-fluoropyrazin-2-yl)-7-fluoro-4-methylisoquinolin-1(2H)-one (30 mg, 0.057 mmol) and N,N-dimethyl-1-(2-(tetrahydro-2H-pyran-4-yl)-5-(4,4,5,5-tetramethyl-1,3,2-dioxaborolan-2-yl)phenyl)methanamine hydrochloride (24.02 mg, 0.063 mmol) to give the title compound (3.3 mg, 11.4% yield) as a tan solid. ¹H NMR (500 MHz, DMSO-d₆) δ ppm 11.27 (br. s., 1H), 7.99 (d, J=10.03 Hz, 1H), 7.83 (d, J=5.99 Hz, 1H), 7.70 (br. s., 2H), 7.38 (d, J=7.58 Hz, 1H), 7.05 (br. s., 1H), 6.88 (br. s., 2H), 3.95 (d, J=8.93 Hz, 2H), 3.44 (br. s., 4H), 3.20 (br. s., 1H), 2.23 (br. s., 3H), 2.15 (br. s., 6H), 1.71 (d, J=10.27 Hz, 2H), 1.57-1.65 (m, 2H); LCMS: [M+H]⁺=506.38.

### Example 236: 6-(3-amino-6-(5-((dimethylamino)methyl)-6-morpholinopyridin-3-yl)-5-fluoropyrazin-2-yl)-4-methylisoquinolin-1(2H)-one (I-236)

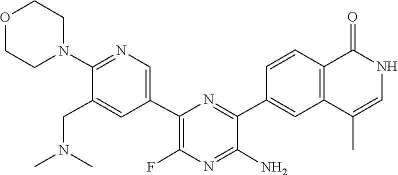 [SMILES: Cc1c[nH]c(=O)c2ccc(-c3nc(-c4cnc(N5CCOCC5)c(CN(C)C)c4)c(F)nc3N)cc12]

Prepared from 6-(3-amino-6-bromo-5-fluoropyrazin-2-yl)-4-methylisoquinolin-1(2H)-one (40 mg, 0.115 mmol) and (5-((dimethylamino)methyl)-6-morpholinopyridin-3-yl)boronic acid (36.4 mg, 0.137 mmol) to give the product (26.8 mg, 47.8% yield) as a grey solid. ¹H NMR (500 MHz, DMSO-d₆) δ ppm 11.14 (d, J=5.38 Hz, 1H), 8.65 (s, 1H), 8.33 (d, J=8.31 Hz, 1H), 8.19 (s, 1H), 7.99 (s, 1H), 7.84 (d, J=8.31 Hz, 1H), 7.07 (d, J=4.52 Hz, 1H), 7.01 (s, 2H), 3.73-3.77 (m, 4H), 3.41 (s, 2H), 3.18-3.22 (m, 4H), 2.26 (s, 3H), 2.19 (s, 6H); LCMS: [M+H]⁺=490.46.

### Example 237: 6-(3-amino-6-(5-((dimethylamino)methyl)-6-morpholinopyridin-3-yl)-5-fluoropyrazin-2-yl)-3,4-dihydroisoquinolin-1(2H)-one (I-237)

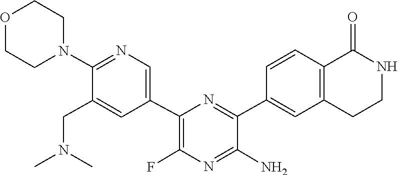 [SMILES: CN(C)Cc1cc(-c2nc(-c3ccc4c(c3)CCNC4=O)c(N)nc2F)cnc1N1CCOCC1]

### Step 1: 5-bromo-2-morpholinonicotinaldehyde

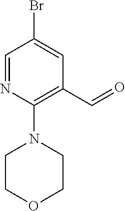 [SMILES: O=Cc1cc(Br)cnc1N1CCOCC1]

To a solution of 5-bromo-2-fluoropyridine-3-carboxaldehyde (1.0 g, 4.90 mmol) and potassium carbonate (1.355 g, 9.80 mmol) in N,N-dimethylformamide (5 ml) was added morpholine (0.444 ml, 5.15 mmol). The reaction was stirred at 80° C. for 2 hours. The mixture was partitioned between water (100 ml) and ethyl acetate (20 ml). The organic phase was separated, and the aqueous phase was washed with ethyl acetate (20 ml). The combined organic phases were washed with water, followed by brine, dried over anhydrous sodium sulfate, and concentrated. The crude was purified by flash sgc eluting with 0-30% EtOAc/Hexanes. The desired fractions were collected, concentrated and dried under vacuum to afford the product (895 mg, 67.3% yield) as a yellow solid. LCMS: [M+H]⁺=271.10.

### Step 2: 1-(5-bromo-2-morpholinopyridin-3-yl)-N,N-dimethylmethanamine

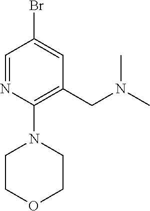 [SMILES: CN(C)Cc1cc(Br)cnc1N1CCOCC1]

To a solution of 5-bromo-2-morpholinonicotinaldehyde (250 mg, 0.922 mmol) in dichloromethane (10 ml) was added dimethylamine, 2.0M solution in THF (1.383 ml, 2.77 mmol) followed by glacial acetic acid (5.27 μl, 0.092 mmol). Upon stirring at RT for 10 minutes, sodium triacetoxyborohydride (586 mg, 2.77 mmol) was added portion-wise and the white suspension was stirred at room temperature for 1 hour. The reaction was basified with 1N NaOH_{(aq)} solution. The organic phase was separated, and the aqueous phase was further washed with DCM (2×). The combined organic phases were washed with brine (1×), dried over anhydrous sodium sulfate and concentrated in vacuo to obtain 1-(5-bromo-2-morpholinopyridin-3-yl)-N,N-dimethylmethanamine (266 mg, 0.886 mmol, 96% yield) as an off-white solid. The material was carried onto the next step without further purification. LCMS [M+H]⁺=300.28.

### Step 3: (5-((dimethylamino)methyl)-6-morpholinopyridin-3-yl)boronic acid

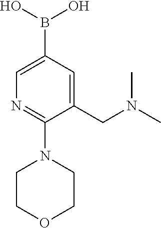 [SMILES: CN(C)Cc1cc(B(O)O)cnc1N1CCOCC1]

Prepared from 1-(5-bromo-2-morpholinopyridin-3-yl)-N,N-dimethylmethanamine (150 mg, 0.500 mmol) to give the boronic acid which was used in the next step without further purification. LCMS: [M+H]+=266.37.

### Step 4: 6-(3-amino-6-(5-((dimethylamino)methyl)-6-morpholinopyridin-3-yl)-5-fluoropyrazin-2-yl)-3,4-dihydroisoquinolin-1(2H)-one

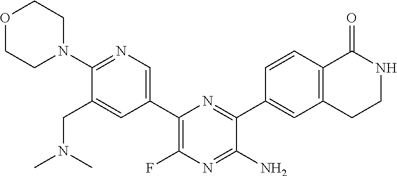 [SMILES: CN(C)Cc1cc(-c2nc(-c3ccc4c(c3)CCNC4=O)c(N)nc2F)cnc1N1CCOCC1]

Prepared from 6-(3-amino-6-bromo-5-fluoropyrazin-2-yl)-3,4-dihydroisoquinolin-1(2H)-one (40 mg, 0.119 mmol) and (5-((dimethylamino)methyl)-6-morpholinopyridin-3-yl)boronic acid (37.7 mg, 0.142 mmol) to give the title compound (30.2 mg, 53.3% yield) as a yellow solid. ¹H NMR (500 MHz, DMSO-d₆) δ ppm 8.64 (s, 1H), 8.14 (s, 1H), 7.98 (br. s., 1H), 7.95 (d, J=7.95 Hz, 1H), 7.68 (d, J=8.19 Hz, 1H), 7.65 (s, 1H), 6.91 (s, 2H), 3.71-3.77 (m, 4H), 3.39-3.45 (m, 4H), 3.17-3.23 (m, 4H), 2.98 (t, J=6.42 Hz, 2H), 2.18 (s, 6H); LCMS [M+H]⁺=478.36.

### Example 238: 6-(3-amino-6-(3-((ethyl(methyl)amino)methyl)-4-(tetrahydro-2H-pyran-4-yl)phenyl)-5-fluoropyrazin-2-yl)-4-fluoroisoquinolin-1(2H)-one (I-238)

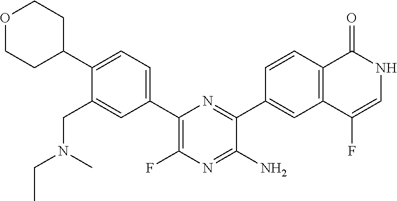 [SMILES: CCN(C)Cc1cc(-c2nc(-c3ccc4c(=O)[nH]cc(F)c4c3)c(N)nc2F)ccc1C1CCOCC1]

Prepared from 6-(3-amino-6-bromo-5-fluoropyrazin-2-yl)-4-fluoroisoquinolin-1(2H)-one (40 mg, 0.113 mmol) and N-methyl-N-(2-(tetrahydro-2H-pyran-4-yl)-5-(4,4,5,5-tetramethyl-1,3,2-dioxaborolan-2-yl)benzyl)ethanamine (44.8 mg, 0.125 mmol) to give the title compound (9.6 mg, 16.8% yield) as a grey solid. ¹H NMR (500 MHz, DMSO-d₆) δ ppm 11.20 (br. s., 1H), 8.32 (d, J=8.19 Hz, 1H), 8.06 (s, 1H), 7.95 (d, J=8.31 Hz, 1H), 7.72-7.77 (m, 2H), 7.44 (d, J=5.62 Hz, 1H), 7.40 (d, J=8.56 Hz, 1H), 6.99 (s, 2H), 3.96 (dd, J=10.76, 3.30 Hz, 2H), 3.52 (s, 2H), 3.43 (t, J=11.19 Hz, 2H), 3.25 (t, J=11.80 Hz, 1H), 2.41 (q, J=7.09 Hz, 2H), 2.10 (s, 3H), 1.67-1.76 (m, 2H), 1.60-1.66 (m, 2H), 1.04 (t, J=7.09 Hz, 3H); LCMS: [M+H]⁺=506.44.

### Example 239: 6-(3-amino-6-(3-((ethyl(methyl)amino)methyl)-4-(tetrahydro-2H-pyran-4-yl)phenyl)-5-fluoropyrazin-2-yl)-4-methylisoquinolin-1(2H)-one (I-239)

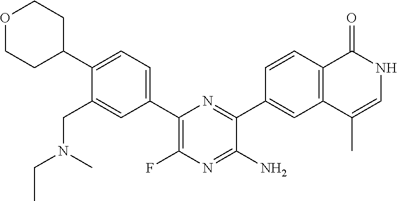 [SMILES: CCN(C)Cc1cc(-c2nc(-c3ccc4c(=O)[nH]cc(C)c4c3)c(N)nc2F)ccc1C1CCOCC1]

Prepared from 6-(3-amino-6-bromo-5-fluoropyrazin-2-yl)-4-methylisoquinolin-1(2H)-one (40 mg, 0.115 mmol) and N-methyl-N-(2-(tetrahydro-2H-pyran-4-yl)-5-(4,4,5,5-tetramethyl-1,3,2-dioxaborolan-2-yl)benzyl)ethanamine (45.3 mg, 0.126 mmol) to give the title compound (28.2 mg, 49.1% yield) as a beige solid. ¹H NMR (500 MHz, DMSO-d₆) δ ppm 11.14 (d, J=5.38 Hz, 1H), 8.33 (d, J=8.31 Hz, 1H), 7.99 (s, 1H), 7.84 (d, J=8.31 Hz, 1H), 7.73-7.79 (m, 2H), 7.39 (d, J=8.80 Hz, 1H), 7.07 (d, J=4.65 Hz, 1H), 6.96 (s, 2H), 3.96 (dd, J=10.76, 3.18 Hz, 2H), 3.51 (s, 2H), 3.43 (t, J=11.00 Hz, 2H), 3.19-3.28 (m, 1H), 2.41 (q, J=7.09 Hz, 2H), 2.26 (s, 3H), 2.10 (s, 3H), 1.68-1.78 (m, 2H), 1.60-1.67 (m, 2H), 1.04 (t, J=7.03 Hz, 3H); LCMS: [M+H]⁺=502.43.

### Example 240: 6-(3-amino-6-(3-((dimethylamino)methyl)-2-fluoro-4-morpholinophenyl)-5-fluoropyrazin-2-yl)-3,4-dihydroisoquinolin-1(2H)-one (I-240)

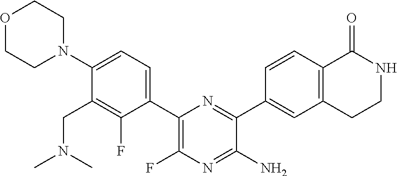 [SMILES: CN(C)Cc1c(N2CCOCC2)ccc(-c2nc(-c3ccc4c(c3)CCNC4=O)c(N)nc2F)c1F]

### Step 1: 2-fluoro-6-morpholinobenzaldehyde

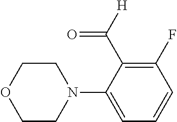 [SMILES: O=Cc1c(F)cccc1N1CCOCC1]

Prepared from 2,6-difluorobenzaldehyde (5 g, 35.2 mmol) and morpholine (3.64 mL, 42.2 mmol). The product was isolated as a pale-yellow oil (3.624 g, 49% yield). LCMS: [M+H]⁺=210.00

### Step 2: 3-bromo-2-fluoro-6-morpholinobenzaldehyde

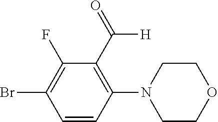 [SMILES: O=Cc1c(N2CCOCC2)ccc(Br)c1F]

To a solution of 2-fluoro-6-morpholinobenzaldehyde (1.50 g, 1.169 mmol) in CH₂Cl₂ (40 ml) kept in an ice bath, was added N-bromosuccinimide (1.531 g, 8.6 mmol) in portions over a period of 5 min. Once addition is complete, the cooling bath was removed, and the reaction mixture was allowed to stir for 75 min at RT. The reaction mixture was concentrated and purified by silica gel chromatography, (eluting with Hexanes containing 0-15% ethyl acetate) to isolate the title compound as a yellow solid (1.69 g, 98%). LCMS: [M+H]⁺=288.25

### Step 3: 1-(3-bromo-2-fluoro-6-morpholinophenyl)-N,N-dimethylmethanamine

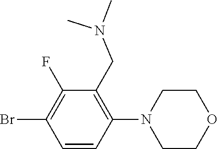 [SMILES: CN(C)Cc1c(N2CCOCC2)ccc(Br)c1F]

Prepared as per the procedure analogous to that of Example 3, step 1 using 3-bromo-2-fluoro-6-morpholinobenzaldehyde (300 mg, 1.041 mmol) Example 58, step 2 and Dimethylamine, 2.0M solution in THF (2.08 ml, 4.16 mmol) as the amine. The title compound was isolated as an off white solid (332 mg, 95% yield). LCMS: [M+H]⁺=325.32

### Step 4: 1-(2-fluoro-6-morpholino-3-(4,4,5,5-tetramethyl-1,3,2-dioxaborolan-2-yl)phenyl)-N,N-dimethylmethanamine

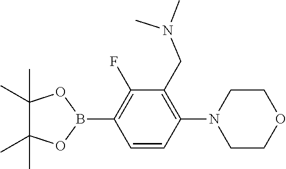 [SMILES: CN(C)Cc1c(N2CCOCC2)ccc(B2OC(C)(C)C(C)(C)O2)c1F]

Prepared from 1-(3-bromo-2-fluoro-6-morpholinophenyl)-N,N-dimethylmethanamine (50 mg, 0.158 mmol) Example 58, step 3. The title compound was isolated as a brown solid which was taken to the next step without any purification. (57 mg, 55% yield based on 86% purity); LCMS: [M+H]⁺=365.35.

### Step 5: 6-(3-amino-6-(3-((dimethylamino)methyl)-2-fluoro-4-morpholinophenyl)-5-fluoropyrazin-2-yl)-3,4-dihydroisoquinolin-1(2H)-one)

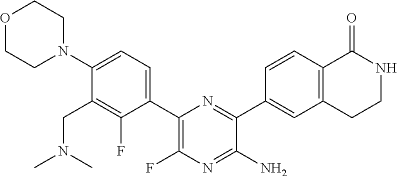 [SMILES: CN(C)Cc1c(N2CCOCC2)ccc(-c2nc(-c3ccc4c(c3)CCNC4=O)c(N)nc2F)c1F]

Prepared from 6-(3-amino-6-bromo-5-fluoropyrazin-2-yl)-3,4-dihydroisoquinolin-1(2H)-one (37.6 mg, 0.112 mmol) and 1-(2-fluoro-6-morpholino-3-(4,4,5,5-tetramethyl-1,3,2-dioxaborolan-2-yl)phenyl)-N,N-dimethylmethanamine (57 mg, 0.134 mmol) to give the title compound as an off-white solid (10.5 mg, 18% yield). ¹H NMR (500 MHz, METHANOL-d) δ=8.04-7.96 (m, 1H), 7.96-7.89 (m, 1H), 7.72-7.65 (m, 1H), 7.64-7.60 (m, 1H), 7.56-7.46 (m, 1H), 7.12-7.02 (m, 1H), 7.00-6.85 (m, 2H), 3.77 (br s, 4H), 3.50 (br s, 2H), 3.41 (br s, 2H), 3.11 (br s, 4H), 2.98 (br d, J=5.6 Hz, 2H), 2.32-2.10 (m, 6H); LCMS: [M+H]⁺=495.41.

### Example 241: 6-(3-amino-6-(3-((dimethylamino)methyl)-2-fluoro-4-morpholinophenyl)-5-fluoropyrazin-2-yl)-7-fluoro-3,4-dihydroisoquinolin-1(2H)-one (I-241)

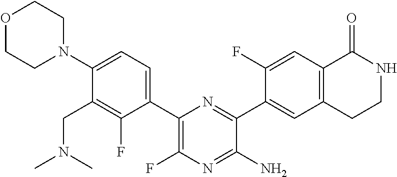 [SMILES: CN(C)Cc1c(N2CCOCC2)ccc(-c2nc(-c3cc4c(cc3F)C(=O)NCC4)c(N)nc2F)c1F]

Prepared from 6-(3-amino-6-bromo-5-fluoropyrazin-2-yl)-7-fluoro-3,4-dihydroisoquinolin-1(2H)-one (41.2 mg, 0.116 mmol) and (3-((dimethylamino)methyl)-2-fluoro-4-morpholinophenyl)boronic acid (50 mg, 0.139 mmol) to give the title compound as an off white solid (8 mg, 13% yield). ¹H NMR (500 MHz, METHANOL-d₄) δ=7.81-7.71 (m, 1H), 7.56-7.46 (m, 2H), 7.17-7.11 (m, 1H), 3.91-3.85 (m, 4H), 3.72 (br s, 2H), 3.60-3.54 (m, 2H), 3.12-3.06 (m, 4H), 3.06-3.02 (m, 2H), 2.37-2.29 (m, 6H); LCMS: [M+H]⁺=513.46.

### Example 242: 6-(3-amino-6-(3-((dimethylamino)methyl)-2-fluoro-4-morpholinophenyl)-5-fluoropyrazin-2-yl)-8-fluoro-3,4-dihydroisoquinolin-1(2H)-one (I-242)

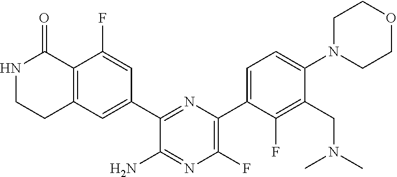 [SMILES: CN(C)Cc1c(N2CCOCC2)ccc(-c2nc(-c3cc(F)c4c(c3)CCNC4=O)c(N)nc2F)c1F]

Prepared from 6-(3-amino-6-bromo-5-fluoropyrazin-2-yl)-8-fluoro-3,4-dihydroisoquinolin-1(2H)-one (41.2 mg, 0.116 mmol) and (3-((dimethylamino)methyl)-2-fluoro-4-morpholinophenyl)boronic acid (50 mg, 0.139 mmol) to give the title compound as a pale-yellow solid (32 mg, 51% yield). ¹H NMR (500 MHz, DMSO-d₆) δ=8.09-7.99 (m, 1H), 7.56-7.48 (m, 1H), 7.48-7.45 (m, 1H), 7.44-7.37 (m, 1H), 7.08-6.95 (m, 3H), 3.84-3.74 (m, 4H), 3.49 (br s, 2H), 3.35 (br d, J=2.4 Hz, 2H), 3.17-3.04 (m, 4H), 2.99-2.92 (m, 2H), 2.28-2.18 (m, 6H); LCMS: [M+H]⁺=513.46.

### Example 243: 6-(3-amino-6-(3-(azetidin-1-ylmethyl)-2-fluoro-4-morpholinophenyl)-5-fluoropyrazin-2-yl)-3,4-dihydroisoquinolin-1(2H)-one (I-243)

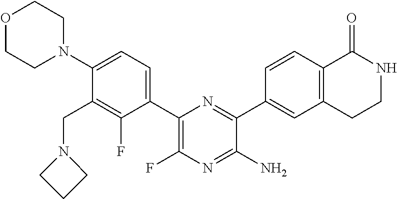 [SMILES: Nc1nc(F)c(-c2ccc(N3CCOCC3)c(CN3CCC3)c2F)nc1-c1ccc2c(c1)CCNC2=O]

Prepared from 6-(3-amino-6-bromo-5-fluoropyrazin-2-yl)-3,4-dihydroisoquinolin-1(2H)-one (36.3 mg, 0.108 mmol) and (3-(azetidin-1-ylmethyl)-2-fluoro-4-morpholinophenyl)boronic acid (50 mg, 0.129 mmol, prepared in turn from a sequence starting with 3-bromo-2-fluoro-6-morpholinobenzaldehyde and azetidine analogous to previous examples) to give the title compound as a beige solid (18 mg, 31% yield). ¹H NMR (500 MHz, METHANOL-d₄) δ 7.9-8.0 (m, 1H), 7.6-7.7 (m, 1H), 7.56 (s, 1H), 7.4-7.5 (m, 1H), 7.0-7.1 (m, 1H), 3.8-3.9 (m, 2H), 3.7-3.8 (m, 4H), 3.4-3.5 (m, 6H), 2.9-3.0 (m, 2H), 2.8-2.9 (m, 4H), 1.9-2.1 (m, 2H); LCMS: [M+H]⁺=507.38.

### Example 244: 6-(3-amino-6-(3-(azetidin-1-ylmethyl)-2-fluoro-4-morpholinophenyl)-5-fluoropyrazin-2-yl)-7-fluoro-3,4-dihydroisoquinolin-1(2H)-one (I-244)

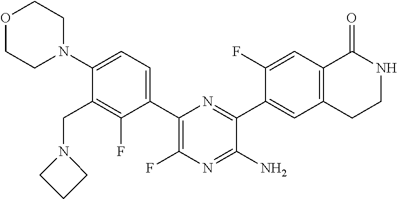 [SMILES: Nc1nc(F)c(-c2ccc(N3CCOCC3)c(CN3CCC3)c2F)nc1-c1cc2c(cc1F)C(=O)NCC2]

Prepared from 6-(3-amino-6-bromo-5-fluoropyrazin-2-yl)-7-fluoro-3,4-dihydroisoquinolin-1(2H)-one (38.2 mg, 0.108 mmol) and (3-(azetidin-1-ylmethyl)-2-fluoro-4-morpholinophenyl)boronic acid (50 mg, 0.129 mmol) to give the title compound as a beige solid (20 mg, 33% yield). ¹H NMR (500 MHz, DMSO-d₆) δ 8.1-8.2 (m, 1H), 7.6-7.7 (m, 1H), 7.5-7.5 (m, 1H), 7.42 (br t, 1H, J=8.4 Hz), 7.0-7.0 (m, 1H), 6.8-7.0 (m, 2H), 3.76 (br s, 4H), 3.6-3.6 (m, 2H), 3.41 (br s, 2H), 3.22 (br t, 4H, J=6.2 Hz), 3.07 (br s, 4H), 2.93 (br t, 2H, J=6.1 Hz), 1.9-1.9 (m, 2H); LCMS: [M+H]⁺=525.37

### Example 245: 6-(3-amino-6-(3-(azetidin-1-ylmethyl)-2-fluoro-4-morpholinophenyl)-5-fluoropyrazin-2-yl)-8-fluoro-3,4-dihydroisoquinolin-1(2H)-one (I-245)

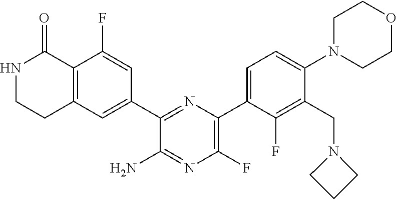 [SMILES: Nc1nc(F)c(-c2ccc(N3CCOCC3)c(CN3CCC3)c2F)nc1-c1cc(F)c2c(c1)CCNC2=O]

Prepared from 6-(3-amino-6-bromo-5-fluoropyrazin-2-yl)-8-fluoro-3,4-dihydroisoquinolin-1(2H)-one (38.2 mg, 0.108 mmol) and (3-(azetidin-1-ylmethyl)-2-fluoro-4-morpholinophenyl)boronic acid (50 mg, 0.129 mmol) to give the title compound as a beige solid (17 mg, 29% yield). ¹H NMR (500 MHz, DMSO-d₆) δ 8.0-8.1 (m, 1H), 7.5-7.5 (m, 2H), 7.4-7.4 (m, 1H), 7.0-7.1 (m, 3H), 3.8-3.8 (m, 4H), 3.6-3.7 (m, 2H), 3.35 (br s, 2H), 3.2-3.3 (m, 4H), 3.0-3.1 (m, 4H), 2.9-3.0 (m, 2H), 1.9-2.0 (m, 2H); LCMS: [M+H]⁺=525.37.

### Example 246: 6-(3-amino-6-(3-((ethyl(methyl)amino)methyl)-2-fluoro-4-morpholinophenyl)-5-fluoropyrazin-2-yl)-7-fluoro-3,4-dihydroisoquinolin-1(2H)-one (I-246)

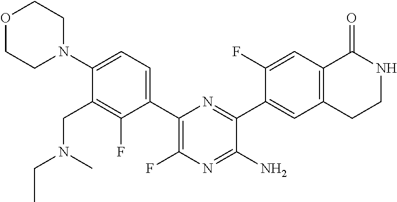 [SMILES: CCN(C)Cc1c(N2CCOCC2)ccc(-c2nc(-c3cc4c(cc3F)C(=O)NCC4)c(N)nc2F)c1F]

Prepared from 6-(3-amino-6-bromo-5-fluoropyrazin-2-yl)-7-fluoro-3,4-dihydroisoquinolin-1(2H)-one (39.6 mg, 0.111 mmol) and (3-((ethyl(methyl)amino)methyl)-2-fluoro-4-morpholinophenyl)boronic acid (45 mg, 0.134 mmol) to give the title compound as a beige solid (18 mg, 29% yield). ¹H NMR (500 MHz, DMSO-d₆) δ 8.15 (br s, 1H), 7.6-7.7 (m, 1H), 7.5-7.5 (m, 1H), 7.4-7.5 (m, 1H), 7.0-7.1 (m, 1H), 6.90 (br s, 2H), 3.7-3.8 (m, 4H), 3.5-3.6 (m, 2H), 3.4-3.4 (m, 2H), 3.1-3.2 (m, 4H), 2.9-3.0 (m, 2H), 2.4-2.5 (m, 2H), 2.16 (br s, 3H), 1.0-1.1 (m, 3H); LCMS: [M+H]⁺=527.44.

### Example 247: 6-(3-amino-6-(3-((ethyl(methyl)amino)methyl)-2-fluoro-4-morpholinophenyl)-5-fluoropyrazin-2-yl)-8-fluoro-3,4-dihydroisoquinolin-1(2H)-one (I-247)

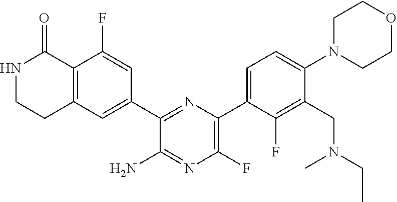 [SMILES: CCN(C)Cc1c(N2CCOCC2)ccc(-c2nc(-c3cc(F)c4c(c3)CCNC4=O)c(N)nc2F)c1F]

Prepared from 6-(3-amino-6-bromo-5-fluoropyrazin-2-yl)-8-fluoro-3,4-dihydroisoquinolin-1(2H)-one (39.6 mg, 0.111 mmol) and (3-((ethyl(methyl)amino)methyl)-2-fluoro-4-morpholinophenyl)boronic acid (45 mg, 0.134 mmol) to give the title compound as a beige solid (21 mg, 33% yield). ¹H NMR (500 MHz, METHANOL-d₄) δ 7.5-7.6 (m, 2H), 7.5-7.5 (m, 1H), 7.1-7.2 (m, 1H), 3.9-3.9 (m, 4H), 3.76 (br s, 2H), 3.5-3.5 (m, 2H), 3.1-3.1 (m, 4H), 3.0-3.1 (m, 2H), 2.6-2.7 (m, 2H), 2.2-2.3 (m, 3H), 1.1-1.2 (m, 3H); LCMS: [M+H]⁺=527.37.

### Example 248: 6-(3-amino-6-(3-((ethyl(methyl)amino)methyl)-2-fluoro-4-morpholinophenyl)-5-fluoropyrazin-2-yl)-3,4-dihydroisoquinolin-1(2H)-one (I-248)

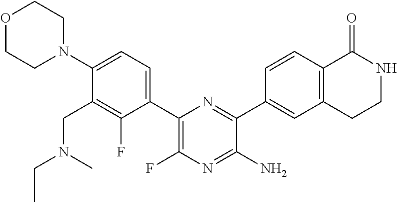 [SMILES: CCN(C)Cc1c(N2CCOCC2)ccc(-c2nc(-c3ccc4c(c3)CCNC4=O)c(N)nc2F)c1F]

Prepared from 6-(3-amino-6-bromo-5-fluoropyrazin-2-yl)-3,4-dihydroisoquinolin-1(2H)-one (37.6 mg, 0.111 mmol) and (3-((ethyl(methyl)amino)methyl)-2-fluoro-4-morpholinophenyl)boronic acid (45 mg, 0.134 mmol, prepared from 3-bromo-2-fluoro-6-morpholinobenzaldehyde and N-ethylmethylamine using a sequence analogous to previous examples) to give the title compound as a white solid (15 mg, 25% yield). ¹H NMR (500 MHz, METHANOL-d₄) δ 7.9-8.0 (m, 1H), 7.6-7.7 (m, 1H), 7.56 (s, 1H), 7.43 (t, 1H, J=8.3 Hz), 7.0-7.1 (m, 1H), 3.7-3.8 (m, 4H), 3.6-3.7 (m, 2H), 3.4-3.5 (m, 2H), 2.97 (br d, 6H, J=5.4 Hz), 2.5-2.5 (m, 2H), 2.2-2.2 (m, 3H), 1.0-1.1 (m, 3H); LCMS: [M+H]+=509.45.

### Example 249: 6-(3-amino-6-(3-((dimethylamino)methyl)-4-methoxyphenyl)-5-fluoropyrazin-2-yl)-7-fluoro-3,4-dihydroisoquinolin-1(2H)-one (I-249)

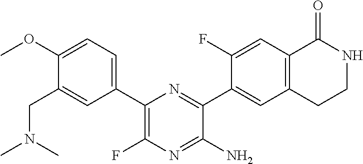 [SMILES: COc1ccc(-c2nc(-c3cc4c(cc3F)C(=O)NCC4)c(N)nc2F)cc1CN(C)C]

Prepared from 6-(3-amino-6-bromo-5-fluoropyrazin-2-yl)-7-fluoro-3,4-dihydroisoquinolin-1(2H)-one (67.5 mg, 0.190 mmol) and 1-(2-methoxy-5-(4,4,5,5-tetramethyl-1,3,2-dioxaborolan-2-yl)phenyl)-N,N-dimethylmethanamine (80 mg, 0.247 mmol) to give the title compound as a beige solid (28 mg, 32% yield). ¹H NMR (500 MHz, DMSO-d₆) δ 8.1-8.2 (m, 1H), 7.8-7.8 (m, 1H), 7.7-7.7 (m, 1H), 7.6-7.7 (m, 1H), 7.5-7.6 (m, 1H), 7.07 (d, 1H, J=8.7 Hz), 6.7-6.8 (m, 2H), 3.8-3.9 (m, 3H), 3.4-3.5 (m, 4H), 2.9-3.0 (m, 2H), 2.1-2.2 (m, 6H); LCMS: [M+H]⁺=440.32.

### Example 250: 6-(3-amino-6-(3-((dimethylamino)methyl)-4-methoxyphenyl)-5-fluoropyrazin-2-yl)-3,4-dihydroisoquinolin-1(2H)-one (I-250)

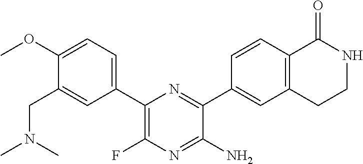 [SMILES: COc1ccc(-c2nc(-c3ccc4c(c3)CCNC4=O)c(N)nc2F)cc1CN(C)C]

### Step 1: 1-(2-methoxy-5-(4,4,5,5-tetramethyl-1,3,2-dioxaborolan-2-yl)phenyl)-N,N-dimethylmethanamine

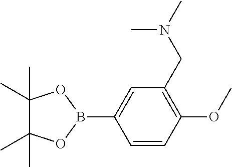

Prepared from 1-(5-bromo-2-methoxyphenyl)-N,N-dimethylmethanamine (225 mg, 0.922 mmol) to give the product as a brown solid (268 mg, 95% yield based on 95% purity); LCMS: [M+H]⁺=292.32.

### Step 2: 6-(3-amino-6-(3-((dimethylamino)methyl)-4-methoxyphenyl)-5-fluoropyrazin-2-yl)-3,4-dihydroisoquinolin-1(2H)-one

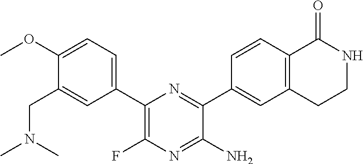 [SMILES: COc1ccc(-c2nc(-c3ccc4c(c3)CCNC4=O)c(N)nc2F)cc1CN(C)C]

Prepared from 6-(3-amino-6-bromo-5-fluoropyrazin-2-yl)-3,4-dihydroisoquinolin-1(2H)-one (64.1 mg, 0.190 mmol) and 1-(2-methoxy-5-(4,4,5,5-tetramethyl-1,3,2-dioxaborolan-2-yl)phenyl)-N,N-dimethylmethanamine (80 mg, 0.247 mmol) to give the title compound as a pale-yellow solid (14 mg, 16% yield). ¹H NMR (500 MHz, DMSO-d₆) δ 8.0-8.0 (m, 1H), 7.9-8.0 (m, 1H), 7.8-7.8 (m, 1H), 7.7-7.8 (m, 1H), 7.7-7.7 (m, 1H), 7.6-7.6 (m, 1H), 7.1-7.1 (m, 1H), 6.8-6.8 (m, 2H), 3.8-3.9 (m, 3H), 3.5-3.5 (m, 2H), 3.4-3.5 (m, 2H), 3.0-3.0 (m, 2H), 2.21 (br s, 6H); LCMS: [M+H]⁺=422.46.

### Example 251: (S)-6-(3-amino-6-(3-(dimethylamino)-2,3-dihydrobenzofuran-5-yl)-5-fluoropyrazin-2-yl)-4-fluoroisoquinolin-1(2H)-one (I-251)

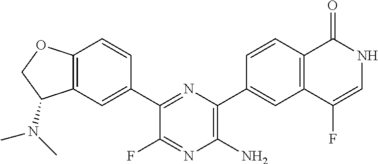 [SMILES: CN(C)[C@@H]1COc2ccc(-c3nc(-c4ccc5c(=O)[nH]cc(F)c5c4)c(N)nc3F)cc21]

### Step 1: (S)-6-(2-amino-5-(3-(dimethylamino)-2,3-dihydrobenzofuran-5-yl)-6-fluoropyridin-3-yl)-4-methylisoquinolin-1(2H)-one

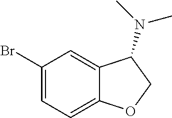

In a similar manner, (S)-5-bromo-N,N-dimethyl-2,3-dihydrobenzofuran-3-amine (0.99 g, 93% yield) was prepared from (S)-5-Bromo-2,3-dihydro-benzofuran-3-ylamine hydrochloride (1.1012 g, 4.40 mmol) reacted with 37% formaldehyde solution in water (1.427 g, 17.58 mmol), sodium acetate (0.361 g, 4.40 mmol) and sodium triacetoxyborohydride (2.79 g, 13.19 mmol) in dichloromethane (20 mL) and methanol (5 mL) at r.t. for 30 min. to give the product (0.99 g, 93% yield). LCMS: [M+H]⁺=242.23

### Step 2: (S)—N,N-dimethyl-5-(4,4,5,5-tetramethyl-1,3,2-dioxaborolan-2-yl)-2,3-dihydrobenzofuran-3-amine

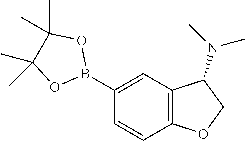

Prepared from (S)-5-bromo-N,N-dimethyl-2,3-dihydrobenzofuran-3-amine (0.99 g, 4.09 mmol) was reacted with bis(pinacolato)diboron (1.246 g, 4.91 mmol), potassium acetate (1.204 g, 12.27 mmol), and [1,1′-bis(diphenylphosphino)ferrocene]dichloropalladium(II) (0.299 g, 0.409 mmol) in 1,4-dioxane (20 mL) at 90-95° C. overnight to give the product (0.94 g, 79% yield). LCMS: [M+H]⁺=290.31

### Step 3: (S)-6-(3-amino-6-(3-(dimethylamino)-2,3-dihydrobenzofuran-5-yl)-5-fluoropyrazin-2-yl)-4-fluoroisoquinolin-1(2H)-one (I-251)

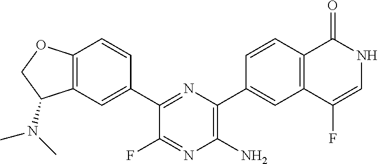 [SMILES: CN(C)[C@@H]1COc2ccc(-c3nc(-c4ccc5c(=O)[nH]cc(F)c5c4)c(N)nc3F)cc21]

Prepared from a (4:1) mixture of 6-(3-amino-6-bromo-5-fluoropyrazin-2-yl)-4-fluoroisoquinolin-1(2H)-one and 6-(3-amino-6-bromo-5-fluoropyrazin-2-yl)-4-chloroisoquinolin-1(2H)-one (100 mg, 0.283 mmol) and (S)—N,N-dimethyl-5-(4,4,5,5-tetramethyl-1,3,2-dioxaborolan-2-yl)-2,3-dihydrobenzofuran-3-amine (82 mg, 0.283 mmol) to give the title compound (30.4 mg, 24.5% yield). ¹H NMR (500 MHz, DMSO-d6) δ=11.21 (br d, J=3.1 Hz, 1H), 8.32 (br d, J=8.3 Hz, 1H), 8.05 (s, 1H), 7.94 (d, J=8.3 Hz, 1H), 7.83 (br s, 1H), 7.77 (br d, J=8.2 Hz, 1H), 7.47-7.41 (m, 1H), 6.96-6.88 (m, 3H), 4.68-4.50 (m, 2H), 4.46-4.36 (m, 1H), 2.14 (br s, 6H); LCMS: [M+H]⁺=436.31.

### Example 252: (S)-6-(3-amino-6-(3-(dimethylamino)-2,3-dihydrobenzofuran-5-yl)-5-fluoropyrazin-2-yl)-4-chloroisoquinolin-1(2H)-one (I-252)

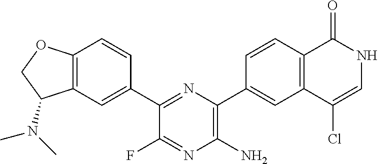 [SMILES: CN(C)[C@@H]1COc2ccc(-c3nc(-c4ccc5c(=O)[nH]cc(Cl)c5c4)c(N)nc3F)cc21]

The purification from the proceeding Example gave the title compound (7.2 mg, 5.3% yield)¹H NMR (500 MHz, DMSO-d₆) δ=11.63 (br d, J=5.0 Hz, 1H), 8.36 (d, J=7.9 Hz, 1H), 8.16 (s, 1H), 7.95 (br d, J=8.3 Hz, 1H), 7.84 (br s, 1H), 7.77 (br d, J=8.6 Hz, 1H), 7.56 (d, J=6.2 Hz, 1H), 6.97-6.86 (m, 3H), 4.69-4.51 (m, 2H), 4.48-4.39 (m, 1H), 2.15 (br s, 6H); δ=−81.23; LCMS: [M+H]⁺=452.24.

### Example 253: (S)-6-(3-amino-6-(3-(dimethylamino)-2,3-dihydrobenzofuran-5-yl)-5-fluoropyrazin-2-yl)-4,8-difluoroisoquinolin-1(2H)-one (I-253)

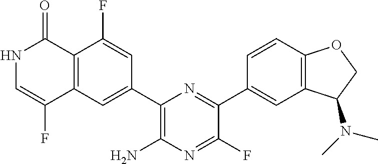 [SMILES: CN(C)[C@@H]1COc2ccc(-c3nc(-c4cc(F)c5c(=O)[nH]cc(F)c5c4)c(N)nc3F)cc21]

Prepared from 6-(3-amino-6-bromo-5-fluoropyrazin-2-yl)-4,8-difluoroisoquinolin-1(2H)-one (25 mg, 0.067 mmol) and (S)—N,N-dimethyl-5-(4,4,5,5-tetramethyl-1,3,2-dioxaborolan-2-yl)-2,3-dihydrobenzofuran-3-amine (19.48 mg, 0.067 mmol) to give the title compound (7.9 mg mmol, 25.3% yield). ¹H NMR (500 MHz, DMSO-d₆) δ=11.19 (br s, 1H), 7.82 (d, J=14.7 Hz, 2H), 7.76 (br d, J=8.8 Hz, 1H), 7.65 (br d, J=12.5 Hz, 1H), 7.51-7.45 (m, 1H), 6.98 (s, 2H), 6.92 (d, J=8.4 Hz, 1H), 4.58 (dd, J=2.6, 10.4 Hz, 1H), 4.53 (br d, J=7.8 Hz, 1H), 4.43-4.37 (m, 1H), 2.12 (s, 6H); LCMS: [M+H]⁺=454.30.

### Example 254: (S)-6-(3-amino-6-(3-(dimethylamino)-2,3-dihydrobenzofuran-5-yl)-5-fluoropyrazin-2-yl)-8-fluoro-3,4-dihydroisoquinolin-1(2H)-one (I-254)

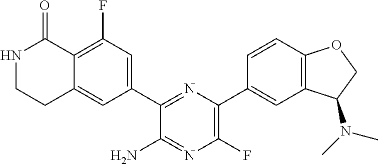 [SMILES: CN(C)[C@@H]1COc2ccc(-c3nc(-c4cc(F)c5c(c4)CCNC5=O)c(N)nc3F)cc21]

Prepared from 6-(3-amino-6-bromo-5-fluoropyrazin-2-yl)-8-fluoro-3,4-dihydroisoquinolin-1(2H)-one (40 mg, 0.113 mmol) reacted with (S)—N,N-dimethyl-5-(4,4,5,5-tetramethyl-1,3,2-dioxaborolan-2-yl)-2,3-dihydrobenzofuran-3-amine (32.6 mg, 0.113 mmol) to give the title compound (30 mg, 59.8% yield). ¹H NMR (500 MHz, DMSO-d₆) δ=8.04 (br s, 1H), 7.78 (s, 1H), 7.75 (d, J=8.3 Hz, 1H), 7.49 (s, 1H), 7.42 (d, J=12.0 Hz, 1H), 6.91 (d, J=8.4 Hz, 1H), 6.87 (s, 2H), 4.58 (dd, J=2.6, 10.0 Hz, 1H), 4.54-4.49 (m, 1H), 4.45-4.34 (m, 1H), 3.40-3.35 (m, 2H), 2.97 (br t, J=6.2 Hz, 2H), 2.12 (s, 6H); LCMS: [M+H]⁺=438.31.

### Example 255: (S)-6-(3-amino-6-(3-(dimethylamino)-2,3-dihydrobenzofuran-5-yl)-5-fluoropyrazin-2-yl)-7-fluoro-3,4-dihydroisoquinolin-1(2H)-one (I-255)

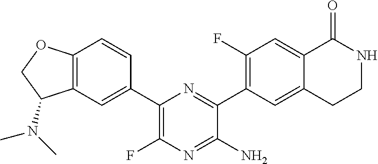 [SMILES: CN(C)[C@@H]1COc2ccc(-c3nc(-c4cc5c(cc4F)C(=O)NCC5)c(N)nc3F)cc21]

Prepared from 6-(3-amino-6-bromo-5-fluoropyrazin-2-yl)-7-fluoro-3,4-dihydroisoquinolin-1(2H)-one (40 mg, 0.113 mmol) reacted with (S)—N,N-dimethyl-5-(4,4,5,5-tetramethyl-1,3,2-dioxaborolan-2-yl)-2,3-dihydrobenzofuran-3-amine (32.6 mg, 0.113 mmol) to afford the title compound (31.8 mg, 63.3% yield). ¹H NMR (500 MHz, DMSO-d₆) δ=8.15 (br s, 1H), 7.75 (s, 1H), 7.70 (br d, J=8.7 Hz, 1H), 7.64 (d, J=10.0 Hz, 1H), 7.52 (d, J=6.7 Hz, 1H), 6.89 (d, J=8.4 Hz, 1H), 6.74 (s, 2H), 4.58 (dd, J=2.8, 10.0 Hz, 1H), 4.54-4.48 (m, 1H), 4.42-4.33 (m, 1H), 3.46-3.39 (m, 2H), 2.95 (br t, J=6.2 Hz, 2H), 2.10 (s, 6H); LCMS: [M+H]⁺=438.31.

### Example 256: (S)-6-(3-amino-6-(3-(dimethylamino)-2,3-dihydrobenzofuran-5-yl)-5-fluoropyrazin-2-yl)-4,4-difluoro-3,4-dihydroisoquinolin-1(2H)-one (I-256)

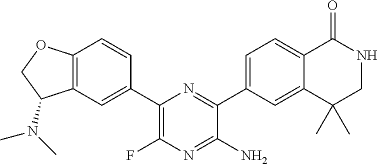 [SMILES: CN(C)[C@@H]1COc2ccc(-c3nc(-c4ccc5c(c4)C(C)(C)CNC5=O)c(N)nc3F)cc21]

Prepared from 6-(3-amino-6-bromo-5-fluoropyrazin-2-yl)-4,4-difluoro-3,4-dihydroisoquinolin-1(2H)-one (40 mg, 0.107 mmol) reacted with (S)—N,N-dimethyl-5-(4,4,5,5-tetramethyl-1,3,2-dioxaborolan-2-yl)-2,3-dihydrobenzofuran-3-amine (31.0 mg, 0.107 mmol) to give the title compound (13.8 mg, 27.5% yield). ¹H NMR (500 MHz, DMSO-d₆) δ=8.52 (br s, 1H), 8.09 (s, 2H), 8.03 (s, 1H), 7.80 (s, 1H), 7.74 (br d, J=8.2 Hz, 1H), 6.92 (d, J=8.4 Hz, 1H), 6.88 (s, 2H), 4.57 (br d, J=10.1 Hz, 1H), 4.53 (br d, J=7.8 Hz, 1H), 4.44-4.36 (m, 1H), 3.96 (br t, J=12.8 Hz, 2H), 2.12 (s, 6H); LCMS: [M+H]⁺=456.30.

### Example 257: 6-(3-amino-6-(3-((dimethylamino)methyl)-4-((2R,6S)-2,6-dimethylmorpholino)phenyl)-5-fluoropyrazin-2-yl)-4,4-difluoro-3,4-dihydroisoquinolin-1(2H)-one (I-257)

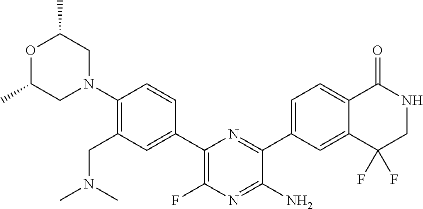 [SMILES: C[C@@H]1CN(c2ccc(-c3nc(-c4ccc5c(c4)C(F)(F)CNC5=O)c(N)nc3F)cc2CN(C)C)C[C@H](C)O1]

Prepared from 6-(3-amino-6-bromo-5-fluoropyrazin-2-yl)-4,4-difluoro-3,4-dihydroisoquinolin-1(2H)-one (30 mg, 0.080 mmol) reacted with 1-(2-(cis-2,6-dimethylmorpholino)-5-(4,4,5,5-tetramethyl-1,3,2-dioxaborolan-2-yl)phenyl)-N,N-dimethylmethanamin hydrochloride (33.0 mg, 0.080 mmol) to give the title compound (23 mg, 51.7% yield). ¹H NMR (500 MHz, DMSO-d₆) δ=8.51 (br s, 1H), 8.09 (s, 2H), 8.03 (s, 1H), 7.87 (s, 1H), 7.71 (br d, J=8.2 Hz, 1H), 7.13 (d, J=8.4 Hz, 1H), 6.92 (s, 2H), 3.96 (br t, J=12.7 Hz, 2H), 3.81-3.69 (m, 2H), 3.44 (s, 2H), 3.17 (br d, J=11.1 Hz, 2H), 2.35 (br t, J=10.8 Hz, 2H), 2.19 (s, 6H), 1.11 (d, J=6.1 Hz, 6H); LCMS: [M+H]⁺=541.22.

### Example 258: 6-(3-amino-6-(5-((dimethylamino)methyl)-2-fluoro-4-morpholinophenyl)-5-fluoropyrazin-2-yl)-3,4-dihydroisoquinolin-1(2H)-one (I-258)

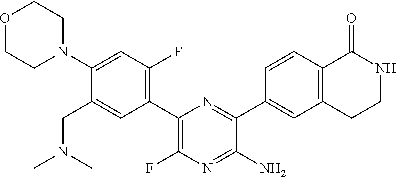 [SMILES: CN(C)Cc1cc(-c2nc(-c3ccc4c(c3)CCNC4=O)c(N)nc2F)c(F)cc1N1CCOCC1]

Prepared from 6-(3-amino-6-bromo-5-fluoropyrazin-2-yl)-3,4-dihydroisoquinolin-1(2H)-one (40 mg, 0.119 mmol) and (5-((dimethylamino)methyl)-2-fluoro-4-morpholinophenyl)boronic acid (40.2 mg, 0.142 mmol, in turn prepared from a sequence starting from 5-bromo-2,4-difluorobenzaldehyde and N,N-diisopropylethylamine analogous to previous Examples) to give the title compound (18.3 mg, 31.2% yield) as a beige solid. ¹H NMR (500 MHz, DMSO-d₆) δ 7.98 (br. s., 1H), 7.93 (d, J=7.95 Hz, 1H), 7.64 (d, J=7.95 Hz, 1H), 7.60 (s, 1H), 7.54 (d, J=8.80 Hz, 1H), 6.98 (d, J=12.23 Hz, 1H), 6.91 (br. s., 2H), 3.75 (br. s., 4H), 3.40 (br. s., 4H), 2.93-3.01 (m, 6H), 2.15 (s, 6H); LCMS [M+H]⁺=495.22.

### Example 259: 6-(3-amino-6-(5-((dimethylamino)methyl)-2-fluoro-4-morpholinophenyl)-5-fluoropyrazin-2-yl)-4-fluoroisoquinolin-1(2H)-one (I-259)

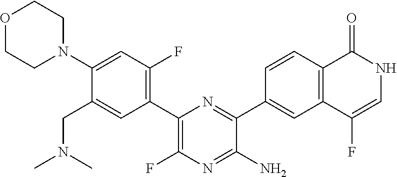 [SMILES: CN(C)Cc1cc(-c2nc(-c3ccc4c(=O)[nH]cc(F)c4c3)c(N)nc2F)c(F)cc1N1CCOCC1]

Prepared from 6-(3-amino-6-bromo-5-fluoropyrazin-2-yl)-4-fluoroisoquinolin-1(2H)-one (40 mg, 0.113 mmol) and (5-((dimethylamino)methyl)-2-fluoro-4-morpholinophenyl)boronic acid (38.3 mg, 0.136 mmol) to give 6-(3-amino-6-(5-((dimethylamino)methyl)-2-fluoro-4-morpholinophenyl)-5-fluoropyrazin-2-yl)-4-fluoroisoquinolin-1(2H)-one (18.1 mg, 31.3% yield) as an off-white solid. ¹H NMR (500 MHz, DMSO-d₆) δ 11.20 (br. s., 1H), 8.30 (d, J=8.31 Hz, 1H), 8.03 (s, 1H), 7.90 (d, J=8.44 Hz, 1H), 7.57 (d, J=8.80 Hz, 1H), 7.44 (d, J=5.87 Hz, 1H), 7.04 (s, 2H), 6.99 (d, J=12.35 Hz, 1H), 3.74 (d, J=4.03 Hz, 4H), 3.40 (s, 2H), 2.98 (br. s., 4H), 2.15 (s, 6H); LCMS: [M+H]⁺=511.45.

### Example 260: 7′-(3-amino-5-fluoro-6-(4-(4-isopropylpiperazin-1-yl)phenyl)pyrazin-2-yl)-2′,3′-dihydro-4′H-spiro[cyclopropane-1,1′-isoquinolin]-4′-one (I-261)

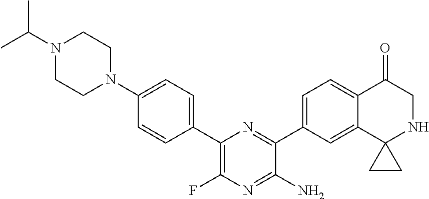 [SMILES: CC(C)N1CCN(c2ccc(-c3nc(-c4ccc5c(c4)C4(CC4)NCC5=O)c(N)nc3F)cc2)CC1]

### Step 1: (2′-(tert-butoxycarbonyl)-4′-oxo-3′,4′-dihydro-2′H-spiro[cyclopropane-1,1′-isoquinolin]-7′-yl)boronic acid

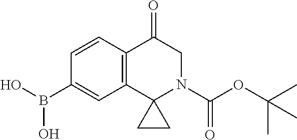 [SMILES: CC(C)(C)OC(=O)N1CC(=O)c2ccc(B(O)O)cc2C12CC2]

Prepared as per the procedure analogous to that of Example 1, step 1, from tert-butyl 7′-bromo-4′-oxo-3′,4′-dihydro-2′H-spiro[cyclopropane-1,1′-isoquinoline]-2′-carboxylate (120 mg, 0.341 mmol). The title compound was isolated as a brown solid which was taken to the next step without any purification. (108 mg, 95% yield based on 95% purity); LCMS: [M+H]+=262.29 (−tBu)

### Step 2: tert-butyl 7′-(3-amino-5-fluoro-6-(4-(4-isopropylpiperazin-1-yl)phenyl)pyrazin-2-yl)-4′-oxo-3′,4′-dihydro-2′H-spiro[cyclopropane-1,1′-isoquinoline]-2′-carboxylate

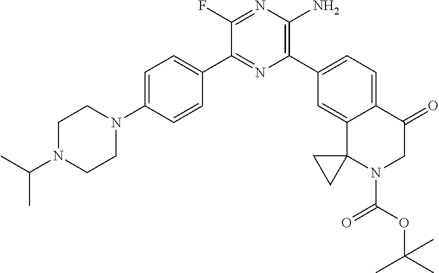 [SMILES: CC(C)N1CCN(c2ccc(-c3nc(-c4ccc5c(c4)C4(CC4)N(C(=O)OC(C)(C)C)CC5=O)c(N)nc3F)cc2)CC1]

A mixture of 3-bromo-6-fluoro-5-(4-(4-isopropylpiperazin-1-yl)phenyl)pyrazin-2-amine (53.1 mg, 0.135 mmol), (2′-(tert-butoxycarbonyl)-4′-oxo-3′,4′-dihydro-2′H-spiro[cyclopropane-1,1′-isoquinolin]-7′-yl)boronic acid (54 mg, 0.162 mmol), XPhos Pd G2 (13.79 mg, 0.018 mmol) and potassium phosphate tribasic reagent grade, (86 mg, 0.404 mmol) in acetonitrile (4 ml) and water (0.8 mL) was heated in an oil bath at 90° C. for 2 h. The reaction mixture was concentrated onto celite and purified by silica gel chromatography, eluting with hexanes containing 0-65% EA afforded the title compound as a beige solid (59 mg, 71% yield). LCMS: [M+H]⁺=587.41

### Step 3: 7′-(3-amino-5-fluoro-6-(4-(4-isopropylpiperazin-1-yl)phenyl)pyrazin-2-yl)-2′,3′-dihydro-4′H-spiro[cyclopropane-1,1′-isoquinolin]-4′-one 0

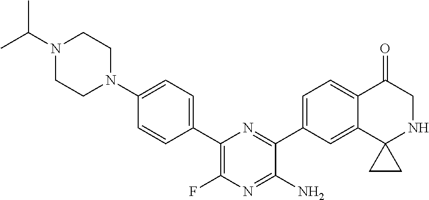 [SMILES: CC(C)N1CCN(c2ccc(-c3nc(-c4ccc5c(c4)C4(CC4)NCC5=O)c(N)nc3F)cc2)CC1]

Prepared from tert-butyl 7′-(3-amino-5-fluoro-6-(4-(4-isopropylpiperazin-1-yl)phenyl)pyrazin-2-yl)-4′-oxo-3′,4′-dihydro-2′H-spiro[cyclopropane-1,1′-isoquinoline]-2′-carboxylate (59 mg, 0.096 mmol) and TFA (0.732 mL, 9.55 mmol) to give the title compound as a beige solid (21 mg, 43% yield). ¹H NMR (500 MHz, DMSO-d₆) δ=8.02-7.93 (m, 1H), 7.80-7.70 (m, 2H), 7.68-7.59 (m, 1H), 7.25-7.16 (m, 1H), 7.01 (br d, J=8.6 Hz, 2H), 6.71 (br s, 2H), 3.83 (br d, J=6.7 Hz, 1H), 3.62-3.49 (m, 2H), 3.19 (br s, 4H), 2.74-2.66 (m, 1H), 2.62-2.55 (m, 4H), 1.30-1.22 (m, 2H), 1.15 (br s, 2H), 1.01 (br d, J=6.4 Hz, 6H); LCMS: [M+H]⁺=487.39.

### Example 261: 6-(3-amino-6-(5-((dimethylamino)methyl)-2-fluoro-4-morpholinophenyl)-5-fluoropyrazin-2-yl)-4-methylisoquinolin-1(2H)-one (I-261)

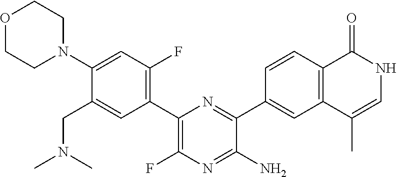 [SMILES: Cc1c[nH]c(=O)c2ccc(-c3nc(-c4cc(CN(C)C)c(N5CCOCC5)cc4F)c(F)nc3N)cc12]

Prepared from 6-(3-amino-6-bromo-5-fluoropyrazin-2-yl)-4-methylisoquinolin-1(2H)-one (37 mg, 0.106 mmol) and (5-((dimethylamino)methyl)-2-fluoro-4-morpholinophenyl)boronic acid (35.9 mg, 0.127 mmol) to give the title compound (0.044 mmol, 41.2% yield) (22.1 mg) as an off white solid. ¹H NMR (500 MHz, DMSO-d₆) δ 11.14 (d, J=4.52 Hz, 1H), 8.31 (d, J=8.31 Hz, 1H), 7.96 (s, 1H), 7.79 (d, J=8.31 Hz, 1H), 7.57 (d, J=8.80 Hz, 1H), 7.06 (d, J=3.06 Hz, 1H), 6.93-7.04 (m, 3H), 3.75 (br. s., 4H), 3.40 (s, 2H), 2.98 (br. s., 4H), 2.24 (s, 3H), 2.16 (s, 6H); LCMS: [M+H]⁺=507.44.

### Example 262: 6-(3-amino-6-(5-((dimethylamino)methyl)-2,3-difluoro-4-morpholinophenyl)-5-fluoropyrazin-2-yl)-3,4-dihydroisoquinolin-1(2H)-one (I-262)

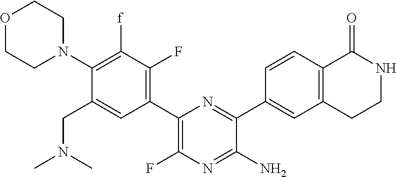

Prepared from 6-(3-amino-6-bromo-5-fluoropyrazin-2-yl)-3,4-dihydroisoquinolin-1(2H)-one (35 mg, 0.104 mmol) and (5-((dimethylamino)methyl)-2,3-difluoro-4-morpholinophenyl)boronic acid (37.4 mg, 0.125 mmol) to give the title compound (11.2 mg, 21% yield, in turn prepared from a sequence starting from 5-bromo-2,3,4-trifluorobenzaldehyde and morpholine using procedures analogous to those described in other Examples) as a grey solid. ¹H NMR (500 MHz, DMSO-d₆) δ 7.99 (br. s., 1H), 7.93 (d, J=7.95 Hz, 1H), 7.63 (d, J=8.07 Hz, 1H), 7.59 (s, 1H), 7.37 (d, J=7.46 Hz, 1H), 7.06 (br. s., 2H), 3.72 (br. s., 4H), 3.48 (s, 2H), 3.38-3.44 (m, 2H), 3.10 (br. s., 4H), 2.96 (t, J=6.42 Hz, 2H), 2.16 (s, 6H); LCMS: [M+H]⁺=513.40.

### Example 263: 6-(3-amino-6-(5-((dimethylamino)methyl)-2,3-difluoro-4-morpholinophenyl)-5-fluoropyrazin-2-yl)-4-fluoroisoquinolin-1(2H)-one (I-263)

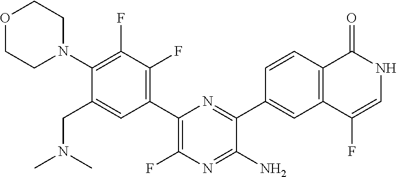 [SMILES: CN(C)Cc1cc(-c2nc(-c3ccc4c(=O)[nH]cc(F)c4c3)c(N)nc2F)c(F)c(F)c1N1CCOCC1]

Prepared from 6-(3-amino-6-bromo-5-fluoropyrazin-2-yl)-4-fluoroisoquinolin-1(2H)-one (35 mg, 0.099 mmol) and (5-((dimethylamino)methyl)-2,3-difluoro-4-morpholinophenyl)boronic acid (35.7 mg, 0.119 mmol) to give the title compound (9.6 mg, 18.3% yield) as a grey solid. ¹H NMR (500 MHz, DMSO-d₆) δ 11.22 (d, J=4.40 Hz, 1H), 8.31 (dd, J=1.34, 8.31 Hz, 1H), 8.02 (s, 1H), 7.89 (d, J=8.31 Hz, 1H), 7.42-7.48 (m, 1H), 7.40 (d, J=7.58 Hz, 1H), 7.18 (s, 2H), 3.72 (br. s., 4H), 3.48 (s, 2H), 3.11 (br. s., 4H), 2.16 (s, 6H); LCMS: [M+H]⁺=529.32.

### Example 264: 6-(3-amino-6-(5-((dimethylamino)methyl)-2,3-difluoro-4-morpholinophenyl)-5-fluoropyrazin-2-yl)-4-methylisoquinolin-1(2H)-one (I-264)

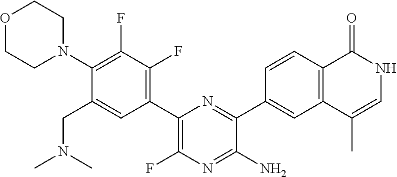 [SMILES: Cc1c[nH]c(=O)c2ccc(-c3nc(-c4cc(CN(C)C)c(N5CCOCC5)c(F)c4F)c(F)nc3N)cc12]

Prepared from 6-(3-amino-6-bromo-5-fluoropyrazin-2-yl)-4-methylisoquinolin-1(2H)-one (35 mg, 0.100 mmol) and (5-((dimethylamino)methyl)-2,3-difluoro-4-morpholinophenyl)boronic acid (36.1 mg, 0.120 mmol) to give the title compound (4.6 mg, 8.8% yield) as a grey solid. ¹H NMR (500 MHz, DMSO-d₆) δ 11.15 (d, J=5.50 Hz, 1H), 8.31 (d, J=8.31 Hz, 1H), 7.95 (s, 1H), 7.77 (d, J=8.31 Hz, 1H), 7.40 (d, J=7.46 Hz, 1H), 7.15 (s, 2H), 7.06 (d, J=5.01 Hz, 1H), 3.72 (br. s., 4H), 3.48 (s, 2H), 3.11 (br. s., 4H), 2.24 (s, 3H), 2.16 (s, 6H); LCMS: [M+H]⁺=525.21.

### Example 265: 6-(3-amino-6-(3-((dimethylamino)methyl)-5-fluoro-4-morpholinophenyl)-5-fluoropyrazin-2-yl)-4-fluoroisoquinolin-1(2H)-one (I-265)

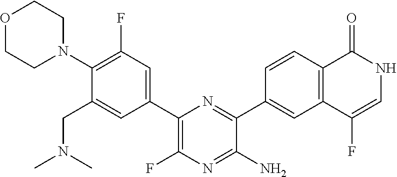 [SMILES: CN(C)Cc1cc(-c2nc(-c3ccc4c(=O)[nH]cc(F)c4c3)c(N)nc2F)cc(F)c1N1CCOCC1]

Prepared from 6-(3-amino-6-bromo-5-fluoropyrazin-2-yl)-4-fluoroisoquinolin-1(2H)-one (40 mg, 0.113 mmol) and (3-((dimethylamino)methyl)-5-fluoro-4-morpholinophenyl)boronic acid (38.3 mg, 0.136 mmol, in turn prepared from a sequence starting from 5-bromo-2,3-difluorobenzaldehyde and morpholine) to give the title compound (19.2 mg, 33.2% yield) as a grey solid. ¹H NMR (500 MHz, DMSO-d₆) δ 11.22 (br. s., 1H), 8.33 (dd, J=1.59, 8.31 Hz, 1H), 8.05 (s, 1H), 7.90-7.96 (m, 1H), 7.73 (s, 1H), 7.53 (d, J=13.94 Hz, 1H), 7.46 (d, J=5.13 Hz, 1H), 7.11 (s, 2H), 3.71 (br. s., 4H), 3.55 (s, 2H), 3.03 (br. s., 4H), 2.19 (s, 6H); LCMS: [M+H]⁺=511.33.

### Example 266: 6-(3-amino-6-(3-((dimethylamino)methyl)-5-fluoro-4-morpholinophenyl)-5-fluoropyrazin-2-yl)-4-methylisoquinolin-1(2H)-one (I-266)

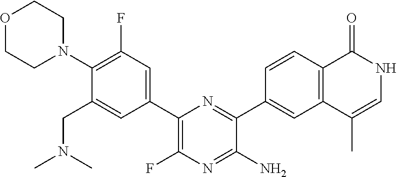 [SMILES: Cc1c[nH]c(=O)c2ccc(-c3nc(-c4cc(F)c(N5CCOCC5)c(CN(C)C)c4)c(F)nc3N)cc12]

Prepared from 6-(3-amino-6-bromo-5-fluoropyrazin-2-yl)-4-methylisoquinolin-1(2H)-one (35 mg, 0.100 mmol) and (3-((dimethylamino)methyl)-5-fluoro-4-morpholinophenyl)boronic acid (33.9 mg, 0.120 mmol) to give the title compound (14.5 mg, 28.6% yield) as a beige solid. ¹H NMR (500 MHz, DMSO-d₆) δ 11.15 (d, J=5.38 Hz, 1H), 8.34 (d, J=8.31 Hz, 1H), 7.98 (s, 1H), 7.83 (d, J=8.19 Hz, 1H), 7.76 (s, 1H), 7.54 (d, J=14.06 Hz, 1H), 7.04-7.12 (m, 3H), 3.71 (br. s., 4H), 3.55 (s, 2H), 3.04 (br. s., 4H), 2.26 (s, 3H), 2.19 (s, 6H); LCMS: [M+H]⁺=507.32.

### Example 267: 6-(3-amino-6-(3-((dimethylamino)methyl)-4-methoxyphenyl)-5-fluoropyrazin-2-yl)-4-fluoroisoquinolin-1(2H)-one (I-267)

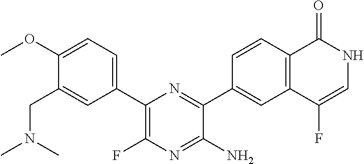 [SMILES: COc1ccc(-c2nc(-c3ccc4c(=O)[nH]cc(F)c4c3)c(N)nc2F)cc1CN(C)C]

Prepared from 6-(3-amino-6-bromo-5-fluoropyrazin-2-yl)-4-fluoroisoquinolin-1(2H)-one (49.5 mg, 0.140 mmol) with 1-(2-methoxy-5-(4,4,5,5-tetramethyl-1,3,2-dioxaborolan-2-yl)phenyl)-N,N-dimethylmethanamine (59 mg, 0.182 mmol) to give the title compound as a beige solid. (28 mg, 43% yield). ¹H NMR (500 MHz, DMSO-d₆) δ 11.1-11.3 (m, 1H), 8.3-8.4 (m, 1H), 8.1-8.1 (m, 1H), 7.9-8.0 (m, 1H), 7.8-7.9 (m, 1H), 7.7-7.8 (m, 1H), 7.4-7.5 (m, 1H), 7.1-7.1 (m, 1H), 6.8-7.0 (m, 2H), 3.8-3.9 (m, 3H), 3.4-3.5 (m, 2H), 2.2-2.2 (m, 6H); LCMS: [M+H]⁺=438.39.

### Example 268: 6-(3-amino-6-(8-((dimethylamino)methyl)chroman-6-yl)-5-fluoropyrazin-2-yl)-4-fluoroisoquinolin-1(2H)-one (I-268)

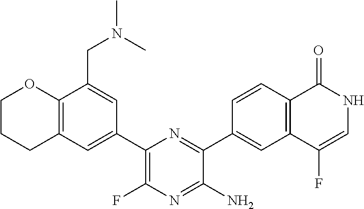 [SMILES: CN(C)Cc1cc(-c2nc(-c3ccc4c(=O)[nH]cc(F)c4c3)c(N)nc2F)cc2c1OCCC2]

### Step 1: 1-(6-bromochroman-8-yl)-N,N-dimethylmethanamine

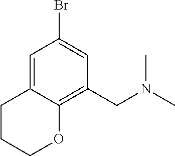

Prepared from 6-bromo-3,4-dihydro-2H-1-benzopyran-8-carbaldehyde (100 mg, 0.415 mmol) and dimethylamine, 2.0M solution in THF (0.622 mL, 1.244 mmol). The product was isolated as a colorless oil (77 mg, 69% yield). LCMS: [M+H]⁺=270.12

### Step 2: N,N-dimethyl-1-(6-(4,4,5,5-tetramethyl-1,3,2-dioxaborolan-2-yl)chroman-8-yl)methanamine

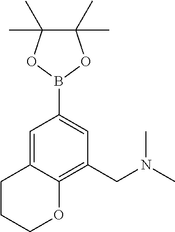 [SMILES: CN(C)Cc1cc(B2OC(C)(C)C(C)(C)O2)cc2c1OCCC2]

Prepared from 1-(6-bromochroman-8-yl)-N,N-dimethylmethanamine (60 mg, 0.211 mmol) to give the title compound as a brown solid which was taken to the next step without any purification. (67 mg, 90% yield based on 90% purity); LCMS: [M+H]⁺=318.11

### Step 3: 6-(3-amino-6-(8-((dimethylamino)methyl)chroman-6-yl)-5-fluoropyrazin-2-yl)-4-fluoroisoquinolin-1(2H)-one

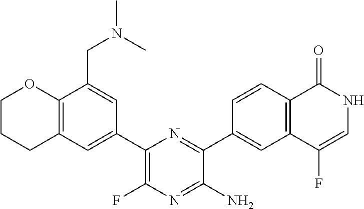 [SMILES: CN(C)Cc1cc(-c2nc(-c3ccc4c(=O)[nH]cc(F)c4c3)c(N)nc2F)cc2c1OCCC2]

Prepared from 6-(3-amino-6-bromo-5-fluoropyrazin-2-yl)-4-fluoroisoquinolin-1(2H)-one (51.6 mg, 0.146 mmol) and N,N-dimethyl-1-(6-(4,4,5,5-tetramethyl-1,3,2-dioxaborolan-2-yl)chroman-8-yl)methanamine (70 mg, 0.190 mmol) to give the title compound as a pale-yellow solid. (27 mg, 38% yield). ¹H NMR (500 MHz, DMSO-d₆) δ 11.1-11.3 (m, 1H), 8.3-8.4 (m, 1H), 8.0-8.1 (m, 1H), 7.9-8.0 (m, 1H), 7.6-7.7 (m, 1H), 7.4-7.5 (m, 2H), 6.88 (s, 2H), 4.2-4.2 (m, 2H), 3.4-3.4 (m, 2H), 2.8-2.9 (m, 2H), 2.1-2.2 (m, 6H), 1.9-2.0 (m, 2H); LCMS: [M+H]⁺=464.28.

### Example 269: 6-(3-amino-6-(8-((dimethylamino)methyl)-2,3-dihydrobenzo[b][1,4]dioxin-6-yl)-5-fluoropyrazin-2-yl)-4-fluoroisoquinolin-1(2H)-one (I-269)

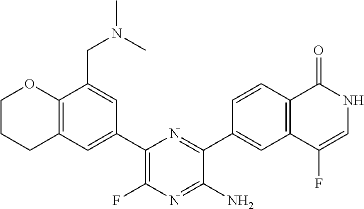 [SMILES: CN(C)Cc1cc(-c2nc(-c3ccc4c(=O)[nH]cc(F)c4c3)c(N)nc2F)cc2c1OCCC2]

Prepared from 6-(3-amino-6-bromo-5-fluoropyrazin-2-yl)-4-fluoroisoquinolin-1(2H)-one (53.9 mg, 0.153 mmol) with N,N-dimethyl-1-(7-(4,4,5,5-tetramethyl-1,3,2-dioxaborolan-2-yl)-2,3-dihydrobenzo[b][1,4]dioxin-5-yl)methanamine (70 mg, 0.198 mmol, in turn prepared using a sequence starting from 7-bromo-2,3-dihydrobenzo[b][1,4]dioxine-5-carbaldehyde and dimethylamine using procedures analogous to previous examples) to give the title compound as a pale-yellow solid (22 mg, 29% yield). ¹H NMR (500 MHz, DMSO-d₆) δ 11.1-11.3 (m, 1H), 8.3-8.4 (m, 1H), 8.0-8.1 (m, 1H), 7.9-8.0 (m, 1H), 7.4-7.5 (m, 2H), 7.2-7.3 (m, 1H), 6.9-7.0 (m, 2H), 4.3-4.3 (m, 2H), 4.27 (br d, 2H, J=4.6 Hz), 3.4-3.5 (m, 2H), 2.18 (s, 6H); LCMS: [M+H]⁺=466.35.

### Example 270: 6-(3-amino-6-(4-(difluoromethoxy)-3-((dimethylamino)methyl)phenyl)-5-fluoropyrazin-2-yl)-4-fluoroisoquinolin-1(2H)-one (I-270)

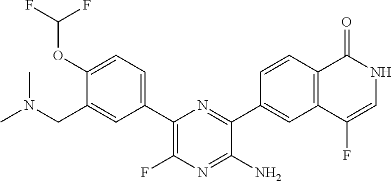 [SMILES: CN(C)Cc1cc(-c2nc(-c3ccc4c(=O)[nH]cc(F)c4c3)c(N)nc2F)ccc1OC(F)F]

Prepared from 6-(3-amino-6-bromo-5-fluoropyrazin-2-yl)-4-fluoroisoquinolin-1(2H)-one (51.0 mg, 0.145 mmol) and 1-(2-(difluoromethoxy)-5-(4,4,5,5-tetramethyl-1,3,2-dioxaborolan-2-yl)phenyl)-N,N-dimethylmethanamine (68 mg, 0.188 mmol, in turn prepared from a sequence starting from 5-bromo-2-(difluoromethoxy)benzaldehyde and dimethylamine analogous to previous examples) to give the title compound as a beige solid (27 mg, 35.5% yield). ¹H NMR (500 MHz, DMSO-d₆) δ 10.9-11.4 (m, 1H), 8.33 (br d, 1H, J=8.2 Hz), 8.0-8.1 (m, 1H), 7.94 (br d, 2H, J=9.8 Hz), 7.8-7.9 (m, 1H), 7.4-7.5 (m, 1H), 7.2-7.4 (m, 2H), 7.0-7.1 (m, 2H), 3.4-3.6 (m, 2H), 2.21 (br s, 6H); LCMS: [M+H]⁺=474.37

### Example 271: 6-(3-amino-6-(3-(2-aminopropan-2-yl)-4-fluorophenyl)-5-fluoropyrazin-2-yl)-3,4-dihydroisoquinolin-1(2H)-one (I-271)

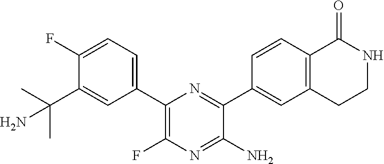 [SMILES: CC(C)(N)c1cc(-c2nc(-c3ccc4c(c3)CCNC4=O)c(N)nc2F)ccc1F]

### Step 1: 2-(5-bromo-2-fluorophenyl)propan-2-ol

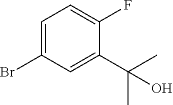

To a 0° C. solution of methyl 5-bromo-2-fluorobenzoate (6.0 g, 25.7 mmol) in anhydrous tetrahydrofuran (50 ml) was added methylmagnesium chloride, 3M in THF (21.46 ml, 64.4 mmol) dropwise. The reaction was warmed to RT and stirred overnight. The solution was poured into an aqueous saturated solution of ammonium chloride and the organic material was extracted with EtOAc (2×). The combined organic layers were dried over anhydrous Na₂SO₄, filtered and concentrated. The crude material was carried onto the next step as-is. LCMS[M+H]⁺ no desired mass observed.

### Step 2: N-(2-(5-bromo-2-fluorophenyl)propan-2-yl)-2-chloroacetamide

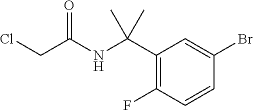 [SMILES: CC(C)(NC(=O)CCl)c1cc(Br)ccc1F]

To a solution of 2-(5-bromo-2-fluorophenyl)propan-2-ol (6.00 g, 25.7 mmol) in chloroacetonitrile 99% (48.9 ml, 772 mmol) was added acetic acid, glacial, 99.8% (4.42 ml, 77 mmol). The reaction was cooled to 0° C. and sulfuric acid (4.41 ml, 82 mmol) was added dropwise. The reaction was warmed to room temperature and stirred overnight. The mixture was then poured into ice and extracted with EtOAc. The organic layer was washed with aqueous 1N NaOH(aq) solution and brine, dried over anhydrous Na₂SO₄, and concentrated to give N-(2-(5-bromo-2-fluorophenyl)propan-2-yl)-2-chloroacetamide (7.94 g, 25.7 mmol, 100%). The crude material was carried onto the next step as-is. LCMS[M+H]⁺=308.03.

### Step 3: 2-(5-bromo-2-fluorophenyl)propan-2-amine

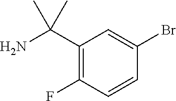

To a solution of N-(2-(5-bromo-2-fluorophenyl)propan-2-yl)-2-chloroacetamide (5.0 g, 16.20 mmol) in ethanol (30 ml) was added concentrated hydrochloric acid (9.91 ml, 405 mmol) and refluxed overnight. The reaction was concentrated in vacuo and partitioned between DCM and 1N NaOH(aq). The organic layer was removed, and the aqueous layer was further washed with DCM (2×). The organic layers were combined, dried over anhydrous sodium sulfate, concentrated, and dried under vacuum to obtain 2-(5-bromo-2-fluorophenyl)propan-2-amine (1.54 g, 40.9% yield) as a yellow oil. LCMS: [M+H]⁺=232.11.

### Step 4: (3-(2-aminopropan-2-yl)-4-fluorophenyl)boronic acid

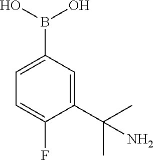

To a mixture of 2-(5-bromo-2-fluorophenyl)propan-2-amine (200 mg, 0.862 mmol), bis(pinacolato)diboron (241 mg, 0.948 mmol), and potassium acetate (254 mg, 2.59 mmol), was added anhydrous 1,4-dioxane (10 ml). The system was degassed and [1,12-bis(diphenylphosphino)ferrocene] dichloropalladium(II) (63.1 mg, 0.086 mmol) was added. The reaction was flushed with nitrogen then heated 100° C. for 2 hours. The mixture was diluted with acetonitrile, filtered through a pad of celite, and concentrated in vacuo to give the boronic acid which was used in the next step without further purification. LCMS: [M+H]+=198.21.

### Step 5: 6-(3-amino-6-(3-(2-aminopropan-2-yl)-4-fluorophenyl)-5-fluoropyrazin-2-yl)-3,4-dihydroisoquinolin-1(2H)-one

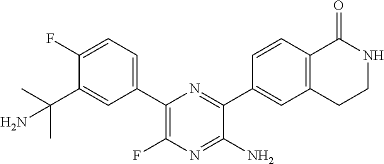 [SMILES: CC(C)(N)c1cc(-c2nc(-c3ccc4c(c3)CCNC4=O)c(N)nc2F)ccc1F]

Prepared from 6-(3-amino-6-bromo-5-fluoropyrazin-2-yl)-3,4-dihydroisoquinolin-1(2H)-one (35 mg, 0.311 mmol) and (3-(2-aminopropan-2-yl)-4-fluorophenyl)boronic acid (61.4 mg, 0.311 mmol) to give the title compound (17.1 mg, 40.2% yield) as a white solid. ¹H NMR (500 MHz, DMSO-d₆) δ 8.14 (d, J=6.60 Hz, 1H), 7.99 (br. s., 1H), 7.95 (d, J=8.07 Hz, 1H), 7.70-7.74 (m, 1H), 7.68 (d, J=8.07 Hz, 1H), 7.65 (s, 1H), 7.20 (dd, J=8.56, 12.10 Hz, 1H), 6.89 (s, 2H), 3.42 (dt, J=2.69, 6.48 Hz, 2H), 2.98 (t, J=6.48 Hz, 2H), 2.04 (br. s., 2H), 1.45 (s, 6H); LCMS: [M+H]⁺=411.25.

### Example 272: 6-(3-amino-6-(3-((dimethylamino)methyl)-4-fluorophenyl)-5-fluoropyrazin-2-yl)-3,4-dihydroisoquinolin-1(2H)-one (I-272)

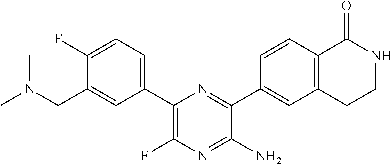 [SMILES: CN(C)Cc1cc(-c2nc(-c3ccc4c(c3)CCNC4=O)c(N)nc2F)ccc1F]

Prepared from 6-(3-amino-6-bromo-5-fluoropyrazin-2-yl)-3,4-dihydroisoquinolin-1(2H)-one (56.9 mg, 0.169 mmol) and 1-(2-fluoro-5-(4,4,5,5-tetramethyl-1,3,2-dioxaborolan-2-yl)phenyl)-N,N-dimethylmethanamine (68 mg, 0.219 mmol) Example 110, step 2. The title compound was isolated as a beige solid. (45 mg, 62% yield). 1H NMR (DMSO-d6, 500 MHz) δ=8.04-7.98 (m, 1H), 7.97-7.93 (m, 1H), 7.89-7.84 (m, 1H), 7.83-7.78 (m, 1H), 7.69-7.65 (m, 1H), 7.65-7.61 (m, 1H), 7.32-7.23 (m, 1H), 6.92 (s, 2H), 3.53-3.46 (m, 2H), 3.45-3.40 (m, 2H), 3.02-2.96 (m, 2H), 2.21-2.14 (m, 6H); LCMS: [M+H]+=410.37

### Example 273: 6-(3-amino-6-(3-((dimethylamino)methyl)-4-fluorophenyl)-5-fluoropyrazin-2-yl)-7-fluoro-3,4-dihydroisoquinolin-1(2H)-one (I-273)

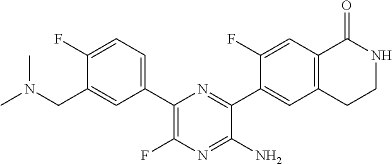 [SMILES: CN(C)Cc1cc(-c2nc(-c3cc4c(cc3F)C(=O)NCC4)c(N)nc2F)ccc1F]

Prepared from 6-(3-amino-6-bromo-5-fluoropyrazin-2-yl)-7-fluoro-3,4-dihydroisoquinolin-1(2H)-one (59.9 mg, 0.169 mmol) and 1-(2-fluoro-5-(4,4,5,5-tetramethyl-1,3,2-dioxaborolan-2-yl)phenyl)-N,N-dimethylmethanamine (68 mg, 0.219 mmol) to give the title compound as a beige solid. (46 mg, 61% yield). 1H NMR (DMSO-d6, 500 MHz) δ=8.23-8.11 (m, 1H), 7.84 (br d, J=6.8 Hz, 1H), 7.80-7.72 (m, 1H), 7.70-7.61 (m, 1H), 7.56-7.49 (m, 1H), 7.26 (t, J=9.2 Hz, 1H), 6.99-6.79 (m, 2H), 3.50-3.45 (m, 2H), 3.45-3.40 (m, 2H), 3.03-2.90 (m, 2H), 2.17 (s, 6H); LCMS: [M+H]⁺=428.36.

### Example 274: 6-(3-amino-6-(3-((dimethylamino)methyl)-4-fluorophenyl)-5-fluoropyrazin-2-yl)-8-fluoro-3,4-dihydroisoquinolin-1(2H)-one (I-274)

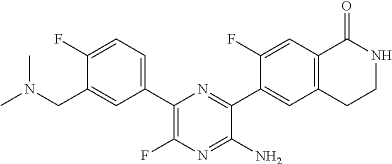 [SMILES: CN(C)Cc1cc(-c2nc(-c3cc4c(cc3F)C(=O)NCC4)c(N)nc2F)ccc1F]

Prepared from 6-(3-amino-6-bromo-5-fluoropyrazin-2-yl)-8-fluoro-3,4-dihydroisoquinolin-1(2H)-one (59.9 mg, 0.169 mmol) with 1-(2-fluoro-5-(4,4,5,5-tetramethyl-1,3,2-dioxaborolan-2-yl)phenyl)-N,N-dimethylmethanamine (68 mg, 0.219 mmol) to give the title compound as a beige solid. (46.5 mg, 61% yield). ¹H NMR (500 MHz, DMSO-d₆) δ=8.09-8.02 (m, 1H), 7.89-7.85 (m, 1H), 7.83-7.78 (m, 1H), 7.51-7.47 (m, 1H), 7.45-7.38 (m, 1H), 7.31-7.23 (m, 1H), 7.06-6.94 (m, 2H), 3.53-3.47 (m, 2H), 3.39-3.34 (m, 2H), 2.97 (t, J=6.3 Hz, 2H), 2.22-2.17 (m, 6H); LCMS: [M+H]⁺=428.36.

### Example 275: 6-(3-amino-6-(3-((dimethylamino)methyl)-4-fluorophenyl)-5-fluoropyrazin-2-yl)-4-methylisoquinolin-1(2H)-one (I-275)

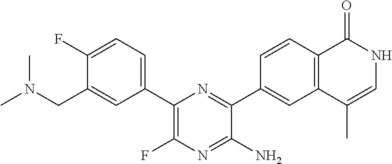 [SMILES: Cc1c[nH]c(=O)c2ccc(-c3nc(-c4ccc(F)c(CN(C)C)c4)c(F)nc3N)cc12]

Prepared from 6-(3-amino-6-bromo-5-fluoropyrazin-2-yl)-4-methylisoquinolin-1(2H)-one (58.9 mg, 0.169 mmol) and 1-(2-fluoro-5-(4,4,5,5-tetramethyl-1,3,2-dioxaborolan-2-yl)phenyl)-N,N-dimethylmethanamine (68 mg, 0.219 mmol) to give the title compound as a pale-yellow solid. (41 mg, 51% yield). ¹H NMR (500 MHz, DMSO-d₆) δ=11.26-11.13 (m, 1H), 8.37-8.32 (m, 1H), 8.03-7.97 (m, 1H), 7.95-7.88 (m, 1H), 7.88-7.78 (m, 2H), 7.29 (br t, J=9.2 Hz, 1H), 7.11-7.07 (m, 1H), 7.06-6.96 (m, 2H), 3.61-3.44 (m, 2H), 2.29-2.26 (m, 3H), 2.25-2.15 (m, 6H); LCMS: [M+H]⁺=422.28.

### Example 276: 6-(3-amino-6-(3-((dimethylamino)methyl)-4-fluorophenyl)-5-fluoropyrazin-2-yl)-4-fluoroisoquinolin-1(2H)-one (I-276)

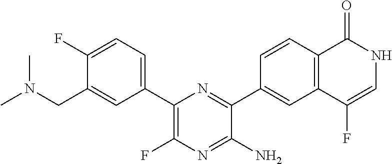 [SMILES: CN(C)Cc1cc(-c2nc(-c3ccc4c(=O)[nH]cc(F)c4c3)c(N)nc2F)ccc1F]

Prepared from 6-(3-amino-6-bromo-5-fluoropyrazin-2-yl)-4-fluoroisoquinolin-1(2H)-one (48.7 mg, 0.138 mmol) and 1-(2-fluoro-5-(4,4,5,5-tetramethyl-1,3,2-dioxaborolan-2-yl)phenyl)-N,N-dimethylmethanamine (53.5 mg, 0.172 mmol) to give the title compound as a beige solid. (25.5 mg, 43% yield). ¹H NMR (500 MHz, DMSO-d₆) δ=11.34-11.09 (m, 1H), 8.39-8.28 (m, 1H), 8.10-8.01 (m, 1H), 7.96-7.91 (m, 1H), 7.91-7.85 (m, 1H), 7.81 (br d, J=5.3 Hz, 1H), 7.49-7.43 (m, 1H), 7.33-7.24 (m, 1H), 7.15-6.91 (m, 2H), 3.54-3.47 (m, 2H), 2.18 (s, 6H); LCMS: [M+H]⁺=426.35.

### Example 277: 6-(3-amino-6-(3-((dimethylamino)methyl)phenyl)-5-fluoropyrazin-2-yl)-3,4-dihydroisoquinolin-1(2H)-one (I-277)

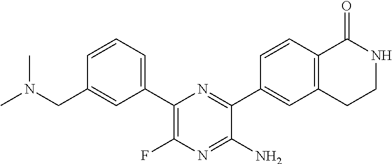 [SMILES: CN(C)Cc1cccc(-c2nc(-c3ccc4c(c3)CCNC4=O)c(N)nc2F)c1]

Prepared from 6-(3-amino-6-bromo-5-fluoropyrazin-2-yl)-3,4-dihydroisoquinolin-1(2H)-one (49.2 mg, 0.146 mmol) and N,N-dimethyl-1-(3-(4,4,5,5-tetramethyl-1,3,2-dioxaborolan-2-yl)phenyl)methanamine (55 mg, 0.190 mmol, prepared by boronylation of (3-bromobenzyl)dimethylamine using methods similar to previous procedures to give the title compound as a beige solid (40 mg, 67% yield). ¹H NMR (500 MHz, DMSO-d₆) δ=8.04-7.99 (m, 1H), 7.98-7.93 (m, 1H), 7.82-7.73 (m, 2H), 7.71-7.66 (m, 1H), 7.66-7.63 (m, 1H), 7.45-7.40 (m, 1H), 7.32-7.24 (m, 1H), 6.91 (s, 2H), 3.47-3.40 (m, 4H), 2.99 (br t, J=6.5 Hz, 2H), 2.21-2.13 (m, 6H); LCMS: [M+H]⁺=392.38.

### Example 278: 6-(3-amino-6-(3-((dimethylamino)methyl)phenyl)-5-fluoropyrazin-2-yl)-7-fluoro-3,4-dihydroisoquinolin-1(2H)-one (I-278)

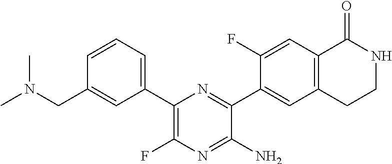 [SMILES: CN(C)Cc1cccc(-c2nc(-c3cc4c(cc3F)C(=O)NCC4)c(N)nc2F)c1]

Prepared from 6-(3-amino-6-bromo-5-fluoropyrazin-2-yl)-7-fluoro-3,4-dihydroisoquinolin-1(2H)-one (51.8 mg, 0.146 mmol) and N,N-dimethyl-1-(3-(4,4,5,5-tetramethyl-1,3,2-dioxaborolan-2-yl)phenyl)methanamine (55 mg, 0.190 mmol) to give the title compound as a beige solid (41.5 mg, 66% yield). ¹H NMR (500 MHz, DMSO-d₆) δ=8.17 (br s, 1H), 7.77-7.73 (m, 1H), 7.73-7.69 (m, 1H), 7.66 (d, J=10.0 Hz, 1H), 7.56-7.50 (m, 1H), 7.40 (t, J=7.6 Hz, 1H), 7.30-7.23 (m, 1H), 6.94-6.81 (m, 2H), 3.47-3.40 (m, 4H), 3.01-2.91 (m, 2H), 2.20-2.12 (m, 6H); LCMS: [M+H]⁺=410.37.

### Example 279: 6-(3-amino-6-(3-((dimethylamino)methyl)phenyl)-5-fluoropyrazin-2-yl)-8-fluoro-3,4-dihydroisoquinolin-1(2H)-one (I-279)

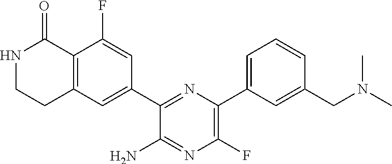 [SMILES: CN(C)Cc1cccc(-c2nc(-c3cc(F)c4c(c3)CCNC4=O)c(N)nc2F)c1]

Prepared from 6-(3-amino-6-bromo-5-fluoropyrazin-2-yl)-8-fluoro-3,4-dihydroisoquinolin-1(2H)-one (51.8 mg, 0.146 mmol) and N,N-dimethyl-1-(3-(4,4,5,5-tetramethyl-1,3,2-dioxaborolan-2-yl)phenyl)methanamine (55 mg, 0.190 mmol) to give the title compound as a beige solid (28 mg, 45% yield). ¹H NMR (500 MHz, DMSO-d₆) δ=8.11-7.99 (m, 1H), 7.79-7.72 (m, 2H), 7.52-7.46 (m, 1H), 7.44-7.37 (m, 2H), 7.31-7.25 (m, 1H), 7.00 (s, 2H), 3.47-3.42 (m, 2H), 3.39-3.35 (m, 2H), 3.01-2.95 (m, 2H), 2.20-2.14 (m, 6H); LCMS: [M+H]⁺=410.31.

### Example 280: 6-(3-amino-6-(3-((dimethylamino)methyl)phenyl)-5-fluoropyrazin-2-yl)-4-methylisoquinolin-1(2H)-one (I-280)

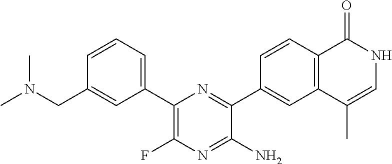 [SMILES: Cc1c[nH]c(=O)c2ccc(-c3nc(-c4cccc(CN(C)C)c4)c(F)nc3N)cc12]

Prepared from 6-(3-amino-6-bromo-5-fluoropyrazin-2-yl)-4-methylisoquinolin-1(2H)-one (50.9 mg, 0.146 mmol) and N,N-dimethyl-1-(3-(4,4,5,5-tetramethyl-1,3,2-dioxaborolan-2-yl)phenyl)methanamine (55 mg, 0.190 mmol) to give the title compound as a beige solid (24.5 mg, 39% yield). ¹H NMR (500 MHz, DMSO-d₆) δ=11.23-11.10 (m, 1H), 8.41-8.29 (m, 1H), 8.01-7.96 (m, 1H), 7.87-7.80 (m, 2H), 7.79-7.75 (m, 1H), 7.45-7.39 (m, 1H), 7.31-7.25 (m, 1H), 7.09-7.06 (m, 1H), 7.04-6.94 (m, 2H), 3.49-3.39 (m, 2H), 2.29-2.24 (m, 3H), 2.21-2.11 (m, 6H); LCMS: [M+H]⁺=404.35.

### Example 281: 6-(3-amino-6-(3-((dimethylamino)methyl)phenyl)-5-fluoropyrazin-2-yl)-4-fluoroisoquinolin-1(2H)-one (I-281)

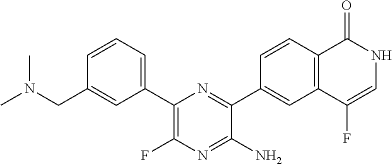 [SMILES: CN(C)Cc1cccc(-c2nc(-c3ccc4c(=O)[nH]cc(F)c4c3)c(N)nc2F)c1]

Prepared from 6-(3-amino-6-bromo-5-fluoropyrazin-2-yl)-4-fluoroisoquinolin-1(2H)-one (49.6 mg, 0.141 mmol) and N,N-dimethyl-1-(3-(4,4,5,5-tetramethyl-1,3,2-dioxaborolan-2-yl)phenyl)methanamine (51 mg, 0.176 mmol) to give the title compound as a beige solid (14 mg, 23% yield). ¹H NMR (500 MHz, DMSO-d₆) δ=11.23 (br s, 1H), 8.40-8.29 (m, 1H), 8.14-8.02 (m, 1H), 8.00-7.92 (m, 1H), 7.84-7.73 (m, 2H), 7.52-7.41 (m, 2H), 7.34-7.27 (m, 1H), 7.16-6.95 (m, 2H), 3.50-3.43 (m, 2H), 2.23-2.08 (m, 6H); LCMS: [M+H]⁺=408.36.

### Example 282: 6-(3-amino-6-(4-(3,6-dihydro-2H-pyran-4-yl)-3-((ethyl(methyl)amino)methyl)phenyl)-5-fluoropyrazin-2-yl)-4-fluoroisoquinolin-1(2H)-one (I-282)

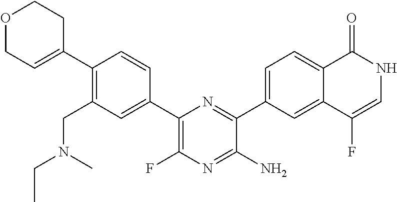 [SMILES: CCN(C)Cc1cc(-c2nc(-c3ccc4c(=O)[nH]cc(F)c4c3)c(N)nc2F)ccc1C1=CCOCC1]

Prepared from 6-(3-amino-6-bromo-5-fluoropyrazin-2-yl)-4-fluoroisoquinolin-1(2H)-one (70 mg, 0.198 mmol) and N-(2-(3,6-dihydro-2H-pyran-4-yl)-5-(4,4,5,5-tetramethyl-1,3,2-dioxaborolan-2-yl)benzyl)-N-methylethanamine hydrochloride (78 mg, 0.198 mmol) to give the title compound (10 mg, 9.5% yield); ¹H NMR (500 MHz, DMSO-d₆) δ=11.21 (br s, 1H), 8.32 (dd, J=1.6, 8.3 Hz, 1H), 8.06 (d, J=1.0 Hz, 1H), 8.00-7.90 (m, 2H), 7.72 (br d, J=7.9 Hz, 1H), 7.44 (br d, J=5.7 Hz, 1H), 7.21 (d, J=8.1 Hz, 1H), 7.03 (s, 2H), 5.69 (br s, 1H), 4.18 (br d, J=2.3 Hz, 2H), 3.81 (t, J=5.3 Hz, 2H), 2.37 (q, J=7.0 Hz, 2H), 2.30 (br s, 2H), 2.11 (s, 3H), 1.00 (t, J=7.1 Hz, 3H); LCMS: [M+H]⁺=504.02.

### Example 283: (S)-6-(3-amino-6-(3-(dimethylamino)-2,3-dihydrobenzofuran-5-yl)-5-fluoropyrazin-2-yl)isoquinolin-1(2H)-one (I-283)

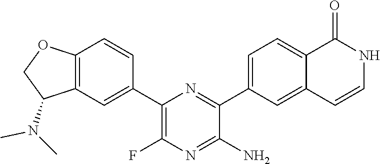 [SMILES: CN(C)[C@@H]1COc2ccc(-c3nc(-c4ccc5c(=O)[nH]ccc5c4)c(N)nc3F)cc21]

Prepared from 6-(3-amino-6-bromo-5-fluoropyrazin-2-yl)isoquinolin-1(2H)-one (34.8 mg, 0.104 mmol) reacted with (S)—N,N-dimethyl-5-(4,4,5,5-tetramethyl-1,3,2-dioxaborolan-2-yl)-2,3-dihydrobenzofuran-3-amine (30 mg, 0.104 mmol), potassium carbonate (43.0 mg, 0.311 mmol), and tetrakis(triphenylphosphine)Palladium(0) (11.99 mg, 10.37 μmol) in 1,4-dioxane (2 ml) and water (0.50 ml) in MW at 120° C. for 90 mins to give the title compound (24.1 mg, 54.6% yield). ¹H NMR (500 MHz, DMSO-d6) d=11.29 (d, J=4.52 Hz, 1H), 8.28 (d, J=8.31 Hz, 1H), 7.99 (s, 1H), 7.74-7.84 (m, 3H), 7.22 (t, J=6.48 Hz, 1H), 6.91 (d, J=8.44 Hz, 1H), 6.83 (s, 2H), 6.64 (d, J=7.09 Hz, 1H), 4.49-4.61 (m, 2H), 4.36-4.43 (m, 1H), 2.12 (s, 6H); LCMS: [M+H]⁺=418.33.

### Example 284: (S)-6-(3-amino-6-(3-(dimethylamino)-2,3-dihydrobenzofuran-5-yl)-5-fluoropyrazin-2-yl)-4-methylisoquinolin-1(2H)-one (I-284)

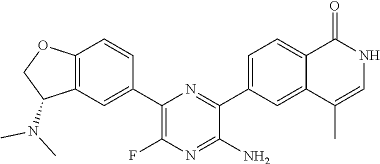 [SMILES: Cc1c[nH]c(=O)c2ccc(-c3nc(-c4ccc5c(c4)[C@H](N(C)C)CO5)c(F)nc3N)cc12]

Prepared from 6-(3-amino-6-bromo-5-fluoropyrazin-2-yl)isoquinolin-1(2H)-one (34.8 mg, 0.104 mmol) reacted with (S)—N,N-dimethyl-5-(4,4,5,5-tetramethyl-1,3,2-dioxaborolan-2-yl)-2,3-dihydrobenzofuran-3-amine (30 mg, 0.104 mmol) to give the title compound (24.1 mg, 54.6% yield); ¹H NMR (500 MHz, DMSO-d₆) d=11.29 (d, J=4.52 Hz, 1H), 8.28 (d, J=8.31 Hz, 1H), 7.99 (s, 1H), 7.74-7.84 (m, 3H), 7.22 (t, J=6.48 Hz, 1H), 6.91 (d, J=8.44 Hz, 1H), 6.83 (s, 2H), 6.64 (d, J=7.09 Hz, 1H), 4.49-4.61 (m, 2H), 4.36-4.43 (m, 1H), 2.12 (s, 6H); LCMS: [M+H]⁺=418.33.

### Example 285: (R)-6-(3-amino-6-(4-(dimethylamino)chroman-6-yl)-5-fluoropyrazin-2-yl)-4-methylisoquinolin-1(2H)-one (I-285)

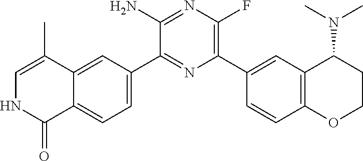 [SMILES: Cc1c[nH]c(=O)c2ccc(-c3nc(-c4ccc5c(c4)[C@H](N(C)C)CCO5)c(F)nc3N)cc12]

Prepared from 6-(3-amino-6-bromo-5-fluoropyrazin-2-yl)-4-methylisoquinolin-1(2H)-one (36.0 mg, 0.103 mmol) reacted with (R)—N,N-dimethyl-6-(4,4,5,5-tetramethyl-1,3,2-dioxaborolan-2-yl)chroman-4-amine hydrochloride (35 mg, 0.103 mmol), tetrakis(triphenylphosphine)Palladium(0) (11.91 mg, 10.30 μmol) and potassium carbonate (57.0 mg, 0.412 mmol) and in 1,4-dioxane (2 ml) and water (0.50 ml) in MW at 120° C. for 90 mins to give the title compound (17.5 mg, 35.9% yield). ¹H NMR (500 MHz, DMSO-d₆) d=11.14 (d, J=5.26 Hz, 1H), 8.32 (d, J=8.31 Hz, 1H), 7.91-8.02 (m, 2H), 7.82 (dd, J=1.41, 8.25 Hz, 1H), 7.65 (d, J=8.68 Hz, 1H), 7.06 (d, J=5.26 Hz, 1H), 6.77-6.88 (m, 3H), 4.30-4.40 (m, 1H), 4.08-4.21 (m, 1H), 3.84 (br. s., 1H), 2.25 (s, 3H), 2.22 (s, 6H), 1.86-2.03 (m, 2H); LCMS: [M+H]⁺=446.29.

### Example 286: (R)-6-(3-amino-6-(4-(dimethylamino)chroman-6-yl)-5-fluoropyrazin-2-yl)isoquinolin-1(2H)-one (I-286)

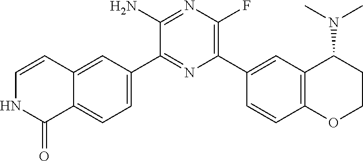 [SMILES: CN(C)[C@@H]1CCOc2ccc(-c3nc(-c4ccc5c(=O)[nH]ccc5c4)c(N)nc3F)cc21]

Prepared from 6-(3-amino-6-bromo-5-fluoropyrazin-2-yl)isoquinolin-1(2H)-one (34.5 mg, 0.103 mmol) reacted with (R)—N,N-dimethyl-6-(4,4,5,5-tetramethyl-1,3,2-dioxaborolan-2-yl)chroman-4-amine hydrochloride (35 mg, 0.103 mmol) to give the title compound (34.5 mg, 0.103 mmol). ¹H NMR (500 MHz, DMSO-d₆) δ 11.29 (d, J=5.50 Hz, 1H), 8.28 (d, J=8.31 Hz, 1H), 7.97 (d, J=1.22 Hz, 1H), 7.92 (s, 1H), 7.78 (dd, J=1.53, 8.25 Hz, 1H), 7.65 (d, J=8.80 Hz, 1H), 7.16-7.26 (m, 1H), 6.77-6.87 (m, 3H), 6.63 (d, J=6.97 Hz, 1H), 4.31-4.39 (m, 1H), 4.09-4.19 (m, 1H), 3.84 (dd, J=5.62, 8.19 Hz, 1H), 2.21 (s, 6H), 1.91-2.00 (m, 2H); LCMS: [M+H]⁺=432.37.

### Example 287: (R)-6-(3-amino-6-(4-(dimethylamino)chroman-6-yl)-5-fluoropyrazin-2-yl)-3,4-dihydroisoquinolin-1(2H)-one (I-287)

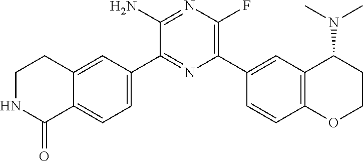 [SMILES: CN(C)[C@@H]1CCOc2ccc(-c3nc(-c4ccc5c(c4)CCNC5=O)c(N)nc3F)cc21]

### Step (S,E)-N-(6-bromochroman-4-ylidene)-2-methylpropane-2-sulfinamide

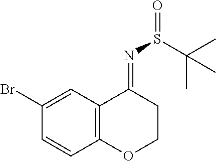 [SMILES: CC(C)(C)[S@](=O)/N=C1\CCOc2ccc(Br)cc21]

6-Bromo-2,3-dihydro-4h-chromen-4-one (5 g, 22.02 mmol) was mixed with (S)-(−)-2-Methyl-2-propanesulfinamide (6.67 g, 55.1 mmol) in Toluene (50 ml), then titanium(IV) ethoxide (5.54 ml, 26.4 mmol) was added. The reaction mixture was stirred at 90° C. for 2 hrs and quenched with ice-water and EtOAc. a lot of solid came out and filtered. The solid was rinsed with EtOAc several time. The organic layer of the filtrate was dried with MgSO4, concentrated with silica gel purified by column chromatography, eluted with 0-30% EtOAc in hexanes to give the product (6.697 g, 92% yield) as a yellow solid. LCMS: [M+H]⁺=330.19.

### Step 2: (S)—N—((S)-6-bromochroman-4-yl)-2-methylpropane-2-sulfinamide and (S)—N—((R)-6-bromochroman-4-yl)-2-methylpropane-2-sulfinamide

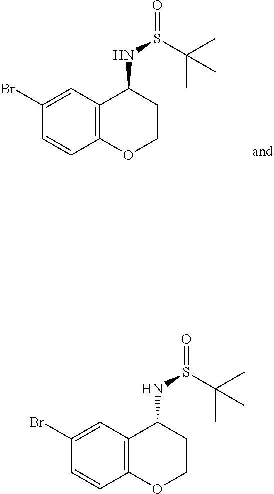 [SMILES: CC(C)(C)[S@](=O)N[C@H]1CCOc2ccc(Br)cc21 ; CC(C)(C)[S@](=O)N[C@@H]1CCOc2ccc(Br)cc21]

(S,E)-N-(6-bromochroman-4-ylidene)-2-methylpropane-2-sulfinamide (3.3 g, 9.99 mmol) was mixed with THF (40 mL) and water (0.6 ml), then was cooled with dry ice-acetone bath (<−50° C.). Sodium borohydride (1.134 g, 30.0 mmol) was added and kept stirring 30 min, then slowly warmed to r.t. for 2 hrs. The reaction mixture quenched with sat-NH₄Cl and extracted with EtOAc. The organic layer was washed with brine, concentrated with silica gel. Purification column chromatography. eluted with 20-25% EtOAc in hexanes to give the product (2.557 g, 77% yield) 890 mg as a white solid. LCMS: [M+H]⁺=332.06.

### Step 3: (R)-6-bromochroman-4-amine hydrochloride

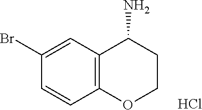 [SMILES: N[C@@H]1CCOc2ccc(Br)cc21 ; Cl]

(R)-6-bromochroman-4-amine hydrochloride (395 mg, 117% yield) was prepared from (S)—N—((R)-6-bromochroman-4-yl)-2-methylpropane-2-sulfinamide (423 mg, 1.273 mmol) reacted with 4.0 M hydrochloric acid in dioxane (3.18 mL, 12.73 mmol) in methanol (5 mL) at r.t. for 90 min. LCMS: [M−HCl—NH₃+H]⁺=211.18.

### Step 4: (R)-6-bromo-N,N-dimethylchroman-4-amine

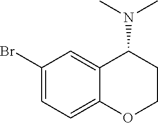

Prepared from (R)-6-bromochroman-4-amine hydrochloride (395 mg, 1.493 mmol) reacted with 37% formaldehyde solution in water (485 mg, 5.97 mmol), sodium acetate (122 mg, 1.493 mmol) and sodium triacetoxyborohydride (949 mg, 4.48 mmol) in methanol (2 ml) and dichloromethane (DCM) (8 ml) at r.t. for 1 h to give the product (374 mg, 98% yield). [M+H]⁺=256.20.

### Step 5: (R)—N,N-dimethyl-6-(4,4,5,5-tetramethyl-1,3,2-dioxaborolan-2-yl)chroman-4-amine hydrochloride

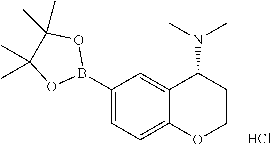

Prepared from (R)-6-bromo-N,N-dimethylchroman-4-amine to give the product (87% yield). LCMS=[M−HCl+H]⁺=304.36.

### Step 6: (R)-6-(3-amino-6-(4-(dimethylamino)chroman-6-yl)-5-fluoropyrazin-2-yl)-3,4-dihydroisoquinolin-1(2H)-one (I-287)

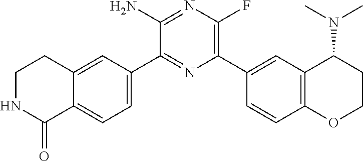 [SMILES: CN(C)[C@@H]1CCOc2ccc(-c3nc(-c4ccc5c(c4)CCNC5=O)c(N)nc3F)cc21]

Prepared from 6-(3-amino-6-bromo-5-fluoropyrazin-2-yl)-3,4-dihydroisoquinolin-1(2H)-one (34.7 mg, 0.103 mmol) and (R)—N,N-dimethyl-6-(4,4,5,5-tetramethyl-1,3,2-dioxaborolan-2-yl)chroman-4-amine hydrochloride (35 mg, 0.103 mmol) to give the title compound (21.3 mg, 47.7% yield). ¹H NMR (500 MHz, DMSO-d₆) δ=7.99 (br s, 1H), 7.95 (d, J=7.9 Hz, 1H), 7.91 (s, 1H), 7.71-7.60 (m, 3H), 6.83 (d, J=8.6 Hz, 1H), 6.77 (s, 2H), 4.41-4.31 (m, 1H), 4.18-4.10 (m, 1H), 3.84 (dd, J=5.9, 8.2 Hz, 1H), 3.42 (dt, J=2.8, 6.6 Hz, 2H), 2.98 (br t, J=6.5 Hz, 2H), 2.22 (s, 6H), 2.03-1.86 (m, 2H); LCMS: [M+H]⁺=434.44.

### Example 288: (R)-6-(3-amino-6-(4-(dimethylamino)chroman-6-yl)-5-fluoropyrazin-2-yl)-7-fluoro-3,4-dihydroisoquinolin-1(2H)-one (I-288)

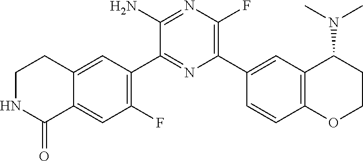 [SMILES: CN(C)[C@@H]1CCOc2ccc(-c3nc(-c4cc5c(cc4F)C(=O)NCC5)c(N)nc3F)cc21]

Prepared from 6-(3-amino-6-bromo-5-fluoropyrazin-2-yl)-7-fluoro-3,4-dihydroisoquinolin-1(2H)-one (36.6 mg, 0.103 mmol) reacted with (R)—N,N-dimethyl-6-(4,4,5,5-tetramethyl-1,3,2-dioxaborolan-2-yl)chroman-4-amine hydrochloride (35 mg, 0.103 mmol), tetrakis(triphenylphosphine)Palladium(0) (11.91 mg, 10.30 μmol) and potassium carbonate (57.0 mg, 0.412 mmol), in 1,4-dioxane (2 ml) and water (0.50 ml) in MW at 120° C. for 90 mins to give the title compound (21.3 mg, 49.0% yield). ¹H NMR (500 MHz, DMSO-d₆) b=8.16 (br s, 1H), 7.87 (s, 1H), 7.64 (d, J=10.1 Hz, 1H), 7.59 (br d, J=8.8 Hz, 1H), 7.50 (d, J=7.0 Hz, 1H), 6.81 (d, J=8.6 Hz, 1H), 6.73 (s, 2H), 4.38-4.29 (m, 1H), 4.16-4.09 (m, 1H), 3.83 (dd, J=5.8, 8.3 Hz, 1H), 3.42 (dt, J=2.6, 6.5 Hz, 2H), 2.95 (br t, J=6.5 Hz, 2H), 2.20 (s, 6H), 2.01-1.88 (m, 2H); LCMS: [M+H]⁺=452.24;

### Example 289: (R)-6-(3-amino-6-(4-(dimethylamino)chroman-6-yl)-5-fluoropyrazin-2-yl)-8-fluoro-3,4-dihydroisoquinolin-1(2H)-one (I-289)

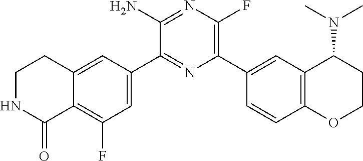 [SMILES: CN(C)[C@@H]1CCOc2ccc(-c3nc(-c4cc(F)c5c(c4)CCNC5=O)c(N)nc3F)cc21]

Prepared from 6-(3-amino-6-bromo-5-fluoropyrazin-2-yl)-8-fluoro-3,4-dihydroisoquinolin-1(2H)-one (36.6 mg, 0.103 mmol) and (R)—N,N-dimethyl-6-(4,4,5,5-tetramethyl-1,3,2-dioxaborolan-2-yl)chroman-4-amine hydrochloride (35 mg, 0.103 mmol) to give the title compound (21.3 mg, 37.1% yield). ¹H NMR (500 MHz, DMSO-d6) δ=8.04 (br s, 1H), 7.90 (s, 1H), 7.64 (br d, J=8.6 Hz, 1H), 7.47 (s, 1H), 7.40 (br d, J=12.0 Hz, 1H), 6.86 (s, 2H), 6.83 (d, J=8.6 Hz, 1H), 4.43-4.30 (m, 1H), 4.19-4.10 (m, 1H), 3.84 (br t, J=6.6 Hz, 1H), 3.43-3.34 (m, 2H), 2.96 (br t, J=6.1 Hz, 2H), 2.22 (s, 6H), 2.05-1.86 (m, 2H); LCMS: [M+H]⁺=452.37.

### Example 290: 6-(3-amino-6-(3-(1-(dimethylamino)ethyl)-4-methoxyphenyl)-5-fluoropyrazin-2-yl)-3,4-dihydroisoquinolin-1(2H)-one (I-290)

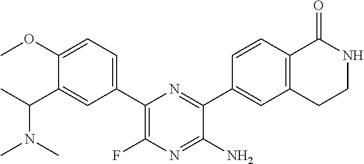 [SMILES: COc1ccc(-c2nc(-c3ccc4c(c3)CCNC4=O)c(N)nc2F)cc1C(C)N(C)C]

### Step 1: 1-(5-bromo-2-methoxyphenyl)-N,N-dimethylethan-1-amine

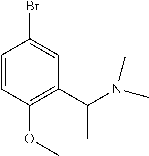

Prepared from 1-(5-bromo-2-methoxyphenyl)ethan-1-one (378 mg, 1.650 mmol) and dimethylamine, 2.0M solution in THF (3.30 ml, 6.60 mmol). The product was isolated as a pale-yellow oil (249 mg, 59% yield). LCMS: [M+H]⁺=258.21

### Step 2: 1-(2-methoxy-5-(4,4,5,5-tetramethyl-1,3,2-dioxaborolan-2-yl)phenyl)-N,N-dimethylethan-1-amine

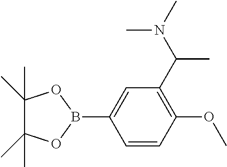

Prepared as per the procedure analogous to that of Example 1, step 1, from 1-(5-bromo-2-methoxyphenyl)-N,N-dimethylethan-1-amine (120 mg, 0.465 mmol) to give the product as a brown solid which was taken to the next step without any purification. (142 mg, 90% yield based on 90% purity). LCMS: [M+H]⁺=306.24

### Step 3: 6-(3-amino-6-(3-(1-(dimethylamino)ethyl)-4-methoxyphenyl)-5-fluoropyrazin-2-yl)-3,4-dihydroisoquinolin-1(2H)-one

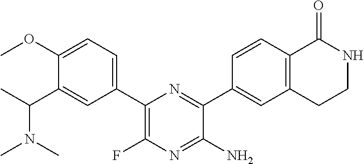 [SMILES: COc1ccc(-c2nc(-c3ccc4c(c3)CCNC4=O)c(N)nc2F)cc1C(C)N(C)C]

Prepared from 6-(3-amino-6-bromo-5-fluoropyrazin-2-yl)-3,4-dihydroisoquinolin-1(2H)-one (53.8 mg, 0.160 mmol) and 1-(2-methoxy-5-(4,4,5,5-tetramethyl-1,3,2-dioxaborolan-2-yl)phenyl)-N,N-dimethylethan-1-amine (65 mg, 0.192 mmol) to give the title compound as a beige solid. (38 mg, 52% yield). ¹H NMR (500 MHz, DMSO-d₆) δ=7.99 (br s, 1H), 7.97-7.92 (m, 1H), 7.89 (s, 1H), 7.74-7.70 (m, 1H), 7.70-7.66 (m, 1H), 7.64 (s, 1H), 7.10-7.06 (m, 1H), 6.82-6.76 (m, 2H), 3.88-3.81 (m, 3H), 3.76-3.68 (m, 1H), 3.47-3.41 (m, 2H), 3.02-2.96 (m, 2H), 2.17-2.11 (m, 6H), 1.25-1.20 (m, 3H); LCMS: [M+H]⁺=466.32.

### Example 291: 6-(3-amino-6-(3-(1-(dimethylamino)ethyl)-4-methoxyphenyl)-5-fluoropyrazin-2-yl)-4-fluoroisoquinolin-1(2H)-one (I-291)

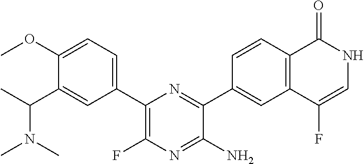 [SMILES: COc1ccc(-c2nc(-c3ccc4c(=O)[nH]cc(F)c4c3)c(N)nc2F)cc1C(C)N(C)C]

Prepared from 6-(3-amino-6-bromo-5-fluoropyrazin-2-yl)-4-fluoroisoquinolin-1(2H)-one (54.1 mg, 0.153 mmol) and 1-(2-methoxy-5-(4,4,5,5-tetramethyl-1,3,2-dioxaborolan-2-yl)phenyl)-N,N-dimethylethan-1-amine (65 mg, 0.192 mmol) to give the title compound as a beige solid (34 mg, 47% yield). ¹H NMR (500 MHz, DMSO-d₆) δ=11.29-11.12 (m, 1H), 8.37-8.28 (m, 1H), 8.12-8.01 (m, 1H), 7.98-7.89 (m, 2H), 7.78-7.69 (m, 1H), 7.50-7.41 (m, 1H), 7.08 (br d, J=8.6 Hz, 1H), 6.98-6.87 (m, 2H), 3.87-3.80 (m, 3H), 3.75-3.67 (m, 1H), 2.18-2.09 (m, 6H), 1.27-1.20 (m, 3H); LCMS: [M+H]⁺=452.24.

### Example 292: 5-(5-amino-3-fluoro-6-(1-oxo-1,2,3,4-tetrahydroisoquinolin-6-yl)pyrazin-2-yl)-2-methoxybenzonitrile (I-292)

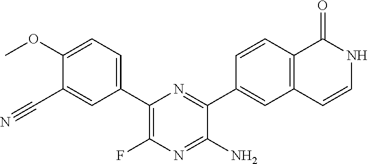 [SMILES: COc1ccc(-c2nc(-c3ccc4c(=O)[nH]ccc4c3)c(N)nc2F)cc1C#N]

Prepared from 6-(3-amino-6-bromo-5-fluoropyrazin-2-yl)-3,4-dihydroisoquinolin-1(2H)-one (40 mg, 0.119 mmol) and 2-methoxy-5-(4,4,5,5-tetramethyl-1,3,2-dioxaborolan-2-yl)benzonitrile (36.9 mg, 0.142 mmol) to give the title compound (41 mg, 89% yield) as an off-white solid. ¹H NMR (500 MHz, DMSO-d₆) δ 8.13-8.17 (m, 1H), 8.11-8.13 (m, 1H), 8.00 (br. s., 1H), 7.95 (d, J=8.07 Hz, 1H), 7.70 (dd, J=1.41, 8.01 Hz, 1H), 7.66 (s, 1H), 7.37 (d, J=8.93 Hz, 1H), 6.99 (s, 2H), 3.96 (s, 3H), 3.42 (dt, J=2.75, 6.51 Hz, 2H), 2.99 (t, J=6.54 Hz, 2H); LCMS: [M+H]⁺=390.37.

### Example 293: 6-(3-amino-6-(3-((dimethylamino)methyl)-4-(isopropylsulfonyl)phenyl)-5-fluoropyrazin-2-yl)-4-fluoroisoquinolin-1(2H)-one (I-293)

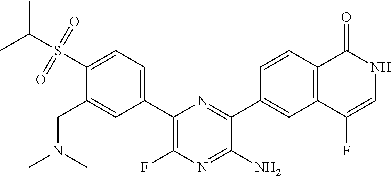 [SMILES: CC(C)S(=O)(=O)c1ccc(-c2nc(-c3ccc4c(=O)[nH]cc(F)c4c3)c(N)nc2F)cc1CN(C)C]

### Step 1: 5-bromo-2-mercaptobenzoic acid

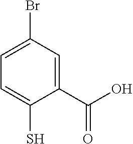 [SMILES: O=C(O)c1cc(Br)ccc1S]

A dilute solution of concentrated HCl (28.0 mL) in ice cooled water (34 mL) was added dropwise to a stirred solution of 2-amino-5-bromobenzoic acid (20 g, 92.6 mmol), NaOH (3.7 g, 92.6 mmol) and sodium nitrite (6.39 g, 92.6 mmol) in water (400 mL) by maintaining the internal temperature between 0-5° C. After completion of the addition, the reaction was stirred at 0° C. for 30 min and then neutralized with potassium acetate (30 g, 306 mmol). The mixture was then treated with a solution of potassium O-ethyl xanthate (44.6 g, 278.0 mmol) in water (223 mL) (preheated to 90° C.) at 90° C. The mixture was stirred for 30 min, cooled to 0° C. and acidified with concentrated HCl (100 mL) and the aqueous layer was decanted and the sticky solid was basified with 10% NaOH solution (200 mL) and stirred to 85° C. for 2 h. To this mixture, NaHSO₃ (9.81 g, 92.6 mmol) was added portionwise and the mass was stirred at 85° C. for 10 min. The mixture was filtered and aqueous layer was cooled to 0° C. and acidified with conc. HCl (100 mL). The precipitate was collected by filtration and washed with water and then n-hexane to afford the product (20.2 g, 85.81 mmol, 51.17%) as a pale brown solid. LCMS: [M+H]⁺=233.08

### Step 2: methyl 5-bromo-2-mercaptobenzoate

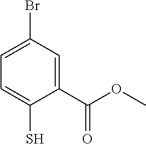

To a cooled solution of 5-bromo-mercaptobenzoic acid (20 g, 85.81 mmol) in methanol (200 mL) at 0° C., conc. H₂SO₄ (40 mL) was added slowly and the reaction mass was stirred at 0° C. for 10 minutes. The mixture was heated at reflux for 16 h, then concentrated under vacuum to get the crude. The crude was diluted with ethyl acetate (300 mL), washed with sat. NaHCO₃ solution (2×500 mL), brine solution (2×200 mL), dried over Na₂SO₄ and concentrated under reduced pressure to obtain the crude product which was purified by column chromatography over silica gel (100-200 mesh) from hexanes to afford the product (8 g, 37.73%) as an orange oil. ¹H NMR (400 MHz, CHLOROFORM-d) δ ppm 8.22 (dd, J=7.9, 1.9 Hz, 1H), 7.8 (d, J=7.8 Hz, 1H), 7.78 (d, J=1.8 Hz, 1H), 3.99 (t, J=7.5 Hz, 4H)).

### Step 3: methyl 5-bromo-2-(isopropylthio)benzoate

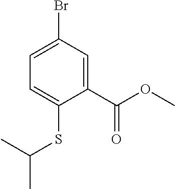

To a solution of methyl 5-bromo-2-mercaptobenzoate (8 g, 32.37 mmol) and K₂CO₃ (13.14 g, 93.1 mmol) in THF (80 mL), 2-bromopropane (4.3 g, 35.61 mmol) was added and the reaction was stirred at reflux temperature for 16 h. The mixture was then diluted with EtOAc (300 mL) and washed with water (2×500 mL). The combined organic layers were dried over Na₂SO₄ and concentrated under reduced pressure to afford the crude product which was purified by column chromatography over silica gel (100-200 mesh) eluting with 0-5% EtOAc in hexanes to afford the product (6 g, 85.45%) as a yellow semi-solid.

### Step 4: (5-bromo-2-(isopropylthio)phenyl)methanol

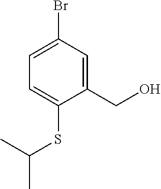

To a solution of methyl 5-bromo-2-(isopropylthio)benzoate (1 g, 3.45 mmol) in THF (10 mL) at 0° C., LAH (1M solution in THF, 3.4 mL, 3.45 mmol) was added dropwise and the reaction was stirred at RT for 3 h. The reaction was diluted with cold water (100 mL) and extracted with EtOAc (2×100 mL). The combined organic layers were dried over Na₂SO₄ and concentrated under reduced pressure to afford the crude product which was purified by sgc eluting with 0-50% EtOAc in hexanes to product (0.8 g, 88.9%) as a light yellow solid of (5-bromo-2-(isopropylthio)phenyl)methanol. ¹H NMR (400 MHz, CHLOROFORM-d) δ ppm 7.6 (s, 1H), 7.40-7.38 (dd, J=8.6, 1.5 Hz, 1H), 7.31-7.28 (dd, J=8.6, 1.5 Hz, 1H), 4.78 (s, 3H), 3.38 (q, J=8.6 Hz, 1H), 1.44-1.32 (s, 6H).

### Step 5: 1-(5-bromo-2-(isopropylthio)phenyl)-N,N-dimethylmethanamine

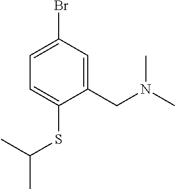

To a cooled solution of (5-bromo-2-(isopropylthio)phenyl)methanol (0.8 g, 3.06 mmol) in DCM (10 mL) at 0° C., TEA (0.9 g, 9.66 mmol) and mesyl chloride (0.3 g, 2.76 mmol) were added dropwise and the reaction was stirred at RT for 1 h. The mixture was diluted with saturated aq. NaHCO₃ (10 mL) and extracted with DCM (2×5 mL). The combined organic layers were dried over Na₂SO₄ and the filtered organic layer treated with TEA (0.97 g, 8.92 mmol) and dimethylamine. HCl (0.7 g, 8.96 mmol) was added at room temperature and the reaction was stirred for 16 h. The mixture was diluted with water (10 mL) and extracted with DCM (3×10 mL). The combined organic layers were dried over Na₂SO₄, concentrated under vacuum to afford the crude product which was purified by column chromatography eluting with 5% MeOH in DCM to afford the product (0.4 g, 1.39 mmol, 45.30%) as a brown solid. LCMS: [M−H]⁺=288.17.

### Step 6: 1-(5-bromo-2-(isopropylsulfonyl)phenyl)-N,N-dimethylmethanamine

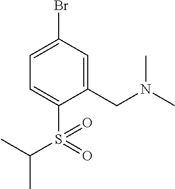 [SMILES: CC(C)S(=O)(=O)c1ccc(Br)cc1CN(C)C]

To a solution of 1-(5-bromo-2-(isopropylthio)phenyl)-N,N-dimethylmethanamine (0.4 g, 1.39 mmol) in DCM (12 mL), mCPBA (0.7 g, 4.16 mmol) was added portionwise at 0° C. and the mixture was stirred for 3 h and quenched with sat·aq. NaHCO₃ (20 mL). The layers were separated and aqueous layer was washed with DCM (2×20 mL). The combined organic layers were dried over Na₂SO₄ and concentrated under reduced to afford the crude product which was purified by sgc eluting with 0-30% EtOAc in hexanes to afford the product (0.25 g, 0.78 mmol, 56.26%) as a white semi-solid. LCMS: [M H]⁺=320.05.

### Step 7: (3-((dimethylamino)methyl)-4-(isopropylsulfonyl)phenyl)boronic acid

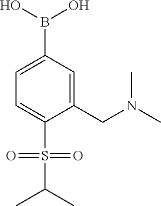 [SMILES: CC(C)S(=O)(=O)c1ccc(B(O)O)cc1CN(C)C]

Prepared from 1-(5-bromo-2-(isopropylsulfonyl)phenyl)-N,N-dimethylmethanamine (100 mg, 0.312 mmol) to give the boronate which was used in the next step without further purification. LCMS: [M+H]+=358.51.

### Step 8: 6-(3-amino-6-(3-((dimethylamino)methyl)-4-(isopropylsulfonyl)phenyl)-5-fluoropyrazin-2-yl)-4-fluoroisoquinolin-1(2H)-one

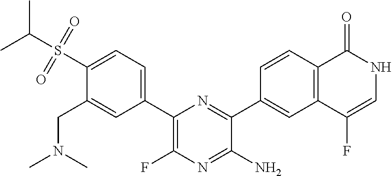 [SMILES: CC(C)S(=O)(=O)c1ccc(-c2nc(-c3ccc4c(=O)[nH]cc(F)c4c3)c(N)nc2F)cc1CN(C)C]

Prepared from 6-(3-amino-6-bromo-5-fluoropyrazin-2-yl)-4-fluoroisoquinolin-1(2H)-one (40 mg, 0.113 mmol) and (3-((dimethylamino)methyl)-4-(isopropylsulfonyl)phenyl)boronic acid (38.8 mg, 0.136 mmol) to give the title compound (17.2 mg, 29.6% yield) as a yellow solid. ¹H NMR (500 MHz, DMSO-d₆) δ 11.23 (br. s., 1H), 8.34 (dd, J=1.59, 8.31 Hz, 1H), 8.13 (s, 1H), 8.01-8.08 (m, 2H), 7.96-7.99 (m, 1H), 7.94 (dd, J=1.47, 8.31 Hz, 1H), 7.46 (br. s., 1H), 7.30 (br. s., 2H), 4.00 (td, J=6.80, 13.66 Hz, 1H), 3.82 (s, 2H), 2.20 (s, 6H), 1.17 (d, J=6.85 Hz, 6H); LCMS: [M+H]⁺=514.36.

### Example 294: 6-(3-amino-6-(2,5-dimethyl-1,2,3,4-tetrahydroisoquinolin-7-yl)-5-fluoropyrazin-2-yl)-3,4-dihydroisoquinolin-1(2H)-one (I-294)

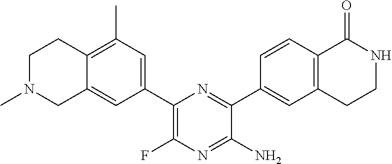 [SMILES: Cc1cc(-c2nc(-c3ccc4c(c3)CCNC4=O)c(N)nc2F)cc2c1CCN(C)C2]

### Step 1: Preparation of 7-bromo-2,5-dimethyl-1,2,3,4-tetrahydroisoquinoline

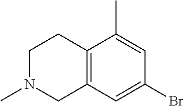

Prepared from 7-bromo-5-methyl-1,2,3,4-tetrahydroisoquinoline (250 mg, 1.106 mmol) and formaldehyde solution, 37% wt in water (0.247 ml, 3.32 mmol) to give the product as a beige solid (270 mg, quantitative yield). LCMS: [M+H]⁺=240.28.

### Step 2: 2,5-dimethyl-7-(4,4,5,5-tetramethyl-1,3,2-dioxaborolan-2-yl)-1,2,3,4-tetrahydroisoquinoline

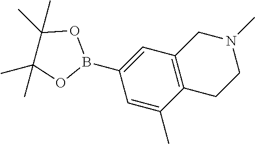

Prepared from 7-bromo-2,5-dimethyl-1,2,3,4-tetrahydroisoquinoline (270 mg, 1.124 mmol) to give the product as a brown solid which was taken to the next step without any purification. (323 mg, 50% yield based on 50% purity). LCMS: [M+H]⁺=288.31

### Step 3: 6-(3-amino-6-(2,5-dimethyl-1,2,3,4-tetrahydroisoquinolin-7-yl)-5-fluoropyrazin-2-yl)-3,4-dihydroisoquinolin-1(2H)-one

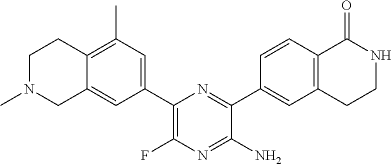 [SMILES: Cc1cc(-c2nc(-c3ccc4c(c3)CCNC4=O)c(N)nc2F)cc2c1CCN(C)C2]

Prepared from 6-(3-amino-6-bromo-5-fluoropyrazin-2-yl)-3,4-dihydroisoquinolin-1(2H)-one (39.1 mg, 0.116 mmol) and 2,5-dimethyl-7-(4,4,5,5-tetramethyl-1,3,2-dioxaborolan-2-yl)-1,2,3,4-tetrahydroisoquinoline (80 mg, 0.139 mmol) to afford the title compound as a beige solid. (9.5 mg, 18% yield). ¹H NMR (500 MHz, DMSO-d₆) δ ppm 7.99 (br. s., 1H), 7.96 (d, J=7.95 Hz, 1H), 7.68 (d, J=8.07 Hz, 1H), 7.64 (s, 1H), 7.50-7.57 (m, 1H), 7.39 (s, 1H), 6.84 (s, 2H), 3.59 (br. s., 2H), 3.43 (d, J=2.08 Hz, 2H), 3.00 (t, J=6.36 Hz, 2H), 2.73 (br. s., 4H), 2.40 (br. s., 3H), 2.24 (s, 3H); LCMS: [M+H]⁺=418.39

### Example 295: 6-(3-amino-6-(2,5-dimethyl-1,2,3,4-tetrahydroisoquinolin-7-yl)-5-fluoropyrazin-2-yl)-4-methylisoquinolin-1(2H)-one (I-295)

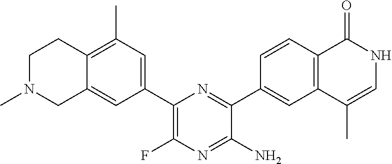 [SMILES: Cc1cc(-c2nc(-c3ccc4c(=O)[nH]cc(C)c4c3)c(N)nc2F)cc2c1CCN(C)C2]

Prepared from 6-(3-amino-6-bromo-5-fluoropyrazin-2-yl)-4-methylisoquinolin-1(2H)-one (40.5 mg, 0.116 mmol) and 2,5-dimethyl-7-(4,4,5,5-tetramethyl-1,3,2-dioxaborolan-2-yl)-1,2,3,4-tetrahydroisoquinoline (80 mg, 0.139 mmol) to give the title compound as a beige solid. (7 mg, 13% yield). ¹H NMR (500 MHz, DMSO-d₆) δ ppm 11.15 (d, J=4.77 Hz, 1H), 8.28-8.47 (m, 1H), 7.94-8.02 (m, 1H), 7.83 (d, J=8.19 Hz, 1H), 7.52 (s, 1H), 7.39 (s, 1H), 7.08 (d, J=5.14 Hz, 1H), 6.93 (br. s., 2H), 3.49-3.55 (m, 2H), 2.70 (d, J=5.38 Hz, 2H), 2.61-2.67 (m, 2H), 2.32-2.38 (m, 3H), 2.26-2.31 (m, 3H), 2.24 (s, 3H); LCMS: [M+H]⁺=430.30.

### Example 296: 6-(3-amino-6-(2,5-dimethyl-1,2,3,4-tetrahydroisoquinolin-7-yl)-5-fluoropyrazin-2-yl)-4-fluoroisoquinolin-1(2H)-one (I-296)

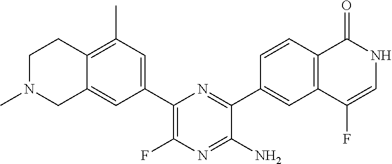 [SMILES: Cc1cc(-c2nc(-c3ccc4c(=O)[nH]cc(F)c4c3)c(N)nc2F)cc2c1CCN(C)C2]

Prepared from 6-(3-amino-6-bromo-5-fluoropyrazin-2-yl)-4-fluoroisoquinolin-1(2H)-one (41.0 mg, 0.116 mmol) and 2,5-dimethyl-7-(4,4,5,5-tetramethyl-1,3,2-dioxaborolan-2-yl)-1,2,3,4-tetrahydroisoquinoline (80 mg, 0.139 mmol) to give the title compound as a beige solid (8 mg, 15% yield). ¹H NMR (500 MHz, DMSO-d₆) δ ppm 11.22 (br. s., 1H), 8.33 (d, J=8.07 Hz, 1H), 8.01-8.10 (m, 1H), 7.94 (d, J=8.19 Hz, 1H), 7.51 (s, 1H), 7.45 (br. s., 1H), 7.38 (s, 1H), 6.89-7.06 (m, 2H), 3.50-3.55 (m, 2H), 2.70 (d, J=5.01 Hz, 2H), 2.65 (d, J=5.26 Hz, 2H), 2.34 (s, 3H), 2.20-2.28 (m, 3H); LCMS: [M+H]⁺=434.25.

### Example 297: 6-(3-amino-6-(3-((ethyl(methyl)amino)methyl)-4-methoxy-5-methylphenyl)-5-fluoropyrazin-2-yl)-3,4-dihydroisoquinolin-1(2H)-one (I-297)

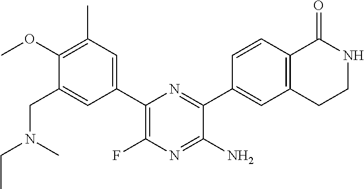 [SMILES: CCN(C)Cc1cc(-c2nc(-c3ccc4c(c3)CCNC4=O)c(N)nc2F)cc(C)c1OC]

Prepared from 6-(3-amino-6-bromo-5-fluoropyrazin-2-yl)-3,4-dihydroisoquinolin-1(2H)-one (47.5 mg, 0.141 mmol) and N-(2-methoxy-3-methyl-5-(4,4,5,5-tetramethyl-1,3,2-dioxaborolan-2-yl)benzyl)-N-methylethanamine (60 mg, 0.169 mmol, in turn prepared from a sequence starting from 5-bromo-2-methoxy-3-methylbenzaldehyde and N-ethylmethylamine) to give the title compound as a beige solid (37 mg, 55.5% yield). ¹H NMR (500 MHz, DMSO-d₆) δ ppm 8.00 (br. s., 1H), 7.96 (d, J=7.83 Hz, 1H), 7.73 (br. s., 1H), 7.67 (d, J=7.95 Hz, 1H), 7.63 (s, 1H), 7.60 (br. s., 1H), 6.85 (br. s., 2H), 3.72 (s, 3H), 3.51-3.59 (m, 2H), 3.43 (br. s., 2H), 2.99 (t, J=5.81 Hz, 2H), 2.44 (br. s., 2H), 2.30 (s, 3H), 2.18 (br. s., 3H), 1.05 (br. s., 3H); LCMS: [M+H]⁺=450.43

### Example 298: 6-(3-amino-6-(3-((ethyl(methyl)amino)methyl)-4-methoxy-5-methylphenyl)-5-fluoropyrazin-2-yl)-4-fluoroisoquinolin-1(2H)-one (I-298)

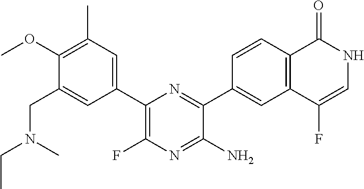 [SMILES: CCN(C)Cc1cc(-c2nc(-c3ccc4c(=O)[nH]cc(F)c4c3)c(N)nc2F)cc(C)c1OC]

Prepared from 6-(3-amino-6-bromo-5-fluoropyrazin-2-yl)-4-fluoroisoquinolin-1(2H)-one (49.8 mg, 0.141 mmol) and N-(2-methoxy-3-methyl-5-(4,4,5,5-tetramethyl-1,3,2-dioxaborolan-2-yl)benzyl)-N-methylethanamine (60 mg, 0.169 mmol) to give the title compound as a beige solid. (48 mg, 69.5% yield). ¹H NMR (500 MHz, DMSO-d₆) δ ppm 11.22 (br. s., 1H), 8.26-8.40 (m, 1H), 8.05 (s, 1H), 7.94 (d, J=8.19 Hz, 1H), 7.75 (br. s., 1H), 7.61 (br. s., 1H), 7.45 (br. s., 1H), 6.97 (br. s., 2H), 3.70-3.77 (m, 3H), 3.51-3.61 (m, 2H), 2.44 (br. s., 2H), 2.28-2.34 (m, 3H), 2.11-2.24 (m, 3H), 1.06 (d, J=12.84 Hz, 3H); LCMS: [M+H]+=466.41.

### Example 299: 6-(3-amino-6-(3-((ethyl(methyl)amino)methyl)-4-methoxy-5-methylphenyl)-5-fluoropyrazin-2-yl)-4-methylisoquinolin-1(2H)-one (I-299)

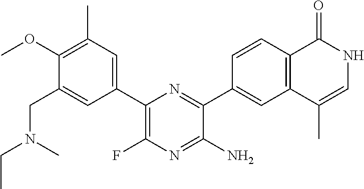 [SMILES: CCN(C)Cc1cc(-c2nc(-c3ccc4c(=O)[nH]cc(C)c4c3)c(N)nc2F)cc(C)c1OC]

Prepared from 6-(3-amino-6-bromo-5-fluoropyrazin-2-yl)-4-methylisoquinolin-1(2H)-one (49.2 mg, 0.141 mmol) and N-(2-methoxy-3-methyl-5-(4,4,5,5-tetramethyl-1,3,2-dioxaborolan-2-yl)benzyl)-N-methylethanamine (60 mg, 0.169 mmol) to give the title compound as a beige solid. (34 mg, 50% yield). ¹H NMR (500 MHz, DMSO-d₆) δ ppm 11.16 (d, J=4.28 Hz, 1H), 8.28-8.39 (m, 1H), 7.99 (s, 1H), 7.81-7.89 (m, 1H), 7.77 (br. s., 1H), 7.62 (br. s., 1H), 7.08 (d, J=4.52 Hz, 1H), 6.95 (br. s., 2H), 3.72 (s, 3H), 3.51-3.60 (m, 2H), 2.39-2.47 (m, 2H), 2.30 (s, 3H), 2.27 (s, 3H), 2.12-2.20 (m, 3H), 1.05 (br. s., 3H); LCMS: [M+H]⁺=462.33.

### Example 300: 6-(3-amino-6-(3-((ethyl(methyl)amino)methyl)-4-methoxyphenyl)-5-fluoropyrazin-2-yl)-4-fluoroisoquinolin-1(2H)-one (I-300)

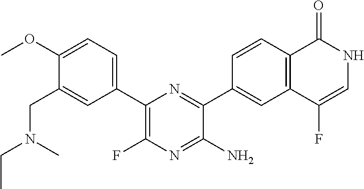 [SMILES: CCN(C)Cc1cc(-c2nc(-c3ccc4c(=O)[nH]cc(F)c4c3)c(N)nc2F)ccc1OC]

Prepared from 6-(3-amino-6-bromo-5-fluoropyrazin-2-yl)-4-fluoroisoquinolin-1(2H)-one (52.1 mg, 0.147 mmol) and N-(2-methoxy-5-(4,4,5,5-tetramethyl-1,3,2-dioxaborolan-2-yl)benzyl)-N-methylethanamine (60 mg, 0.177 mmol, in turn prepared from a sequence starting from 5-bromo-2-methoxybenzaldehyde and N-ethylmethylamine) to give the title compound as a beige solid (40 mg, 57% yield). ¹H NMR (500 MHz, DMSO-d₆) δ ppm 11.22 (br. s., 1H), 8.32 (d, J=8.07 Hz, 1H), 8.07 (s, 1H), 7.96 (d, J=8.19 Hz, 1H), 7.91 (br. s., 1H), 7.77 (d, J=6.48 Hz, 1H), 7.45 (br. s., 1H), 7.10 (d, J=8.07 Hz, 1H), 6.93 (br. s., 2H), 3.83 (br. s., 3H), 3.53 (br. s., 2H), 2.38-2.47 (m, 2H), 2.20 (br. s., 3H), 1.05 (br. s., 3H); LCMS: [M+H]⁺=452.37.

### Example 301: 6-(3-amino-6-(3-((ethyl(methyl)amino)methyl)-4-methoxyphenyl)-5-fluoropyrazin-2-yl)-4-methylisoquinolin-1(2H)-one (I-301)

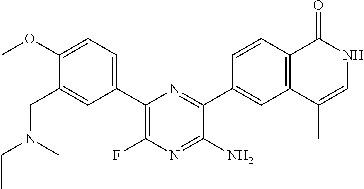 [SMILES: CCN(C)Cc1cc(-c2nc(-c3ccc4c(=O)[nH]cc(C)c4c3)c(N)nc2F)ccc1OC]

Prepared from 6-(3-amino-6-bromo-5-fluoropyrazin-2-yl)-4-methylisoquinolin-1(2H)-one (51.5 mg, 0.147 mmol) and N-(2-methoxy-5-(4,4,5,5-tetramethyl-1,3,2-dioxaborolan-2-yl)benzyl)-N-methylethanamine (60 mg, 0.177 mmol) to give the title compound as a pale-yellow solid. (34 mg, 49% yield). ¹H NMR (500 MHz, DMSO-d₆) δ ppm 11.15 (d, J=5.26 Hz, 1H), 8.33 (d, J=8.31 Hz, 1H), 8.00 (s, 1H), 7.92 (s, 1H), 7.85 (d, J=8.31 Hz, 1H), 7.77 (d, J=7.82 Hz, 1H), 7.02-7.13 (m, 2H), 6.89 (br. s., 2H), 3.83 (s, 3H), 3.50 (br. s., 2H), 2.36-2.43 (m, 2H), 2.27 (s, 3H), 2.08-2.20 (m, 3H), 1.03 (d, J=6.24 Hz, 3H); LCMS: [M+H]⁺=448.42.

### Example 302: 6-(3-amino-6-(3-(1-(dimethylamino)ethyl)-4-hydroxyphenyl)-5-fluoropyrazin-2-yl)-4-fluoroisoquinolin-1(2H)-one (I-302)

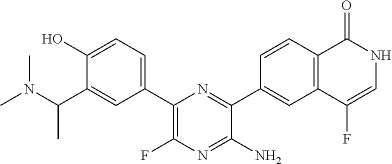 [SMILES: CC(c1cc(-c2nc(-c3ccc4c(=O)[nH]cc(F)c4c3)c(N)nc2F)ccc1O)N(C)C]

Prepared from 6-(3-amino-6-bromo-5-fluoropyrazin-2-yl)-4-fluoroisoquinolin-1(2H)-one (50.0 mg, 0.142 mmol) and 2-(1-(dimethylamino)ethyl)-4-(4,4,5,5-tetramethyl-1,3,2-dioxaborolan-2-yl)phenol (55 mg, 0.170 mmol, in turn prepared from a sequence starting from 5′-bromo-2′-hydroxyacetophenone 98% and dimethylamine, 2.0 M solution in THF analogous to previous examples) to give the title compound as a pale-yellow solid. (23 mg, 35% yield). ¹H NMR (500 MHz, DMSO-d₆) δ ppm 11.22 (br. s., 1H), 8.30-8.37 (m, 1H), 8.06 (s, 1H), 7.95 (dd, J=8.38, 1.16 Hz, 1H), 7.62-7.70 (m, 2H), 7.45 (d, J=4.77 Hz, 1H), 6.85-6.96 (m, 1H), 6.87 (s, 2H), 6.83 (d, J=8.31 Hz, 1H), 3.79 (br. s., 1H), 2.31 (br. s., 6H), 1.35 (d, J=6.48 Hz, 3H); LCMS: [M+H]⁺=438.33.

### Example 303: 6-(3-amino-6-(3-((ethyl(methyl)amino)methyl)-4-methoxy-5-methylphenyl)-5-fluoropyrazin-2-yl)-7-fluoro-3,4-dihydroisoquinolin-1(2H)-one (I-303)

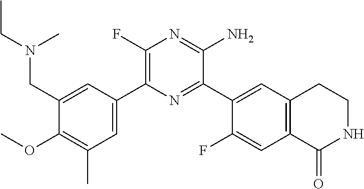 [SMILES: CCN(C)Cc1cc(-c2nc(-c3cc4c(cc3F)C(=O)NCC4)c(N)nc2F)cc(C)c1OC]

Prepared from 6-(3-amino-6-bromo-5-fluoropyrazin-2-yl)-7-fluoro-3,4-dihydroisoquinolin-1(2H)-one (50.1 mg, 0.141 mmol) and N-(2-methoxy-3-methyl-5-(4,4,5,5-tetramethyl-1,3,2-dioxaborolan-2-yl)benzyl)-N-methylethanamine (60 mg, 0.169 mmol) to give the title compound as a beige solid. (26 mg, 37.5% yield); ¹H NMR (500 MHz, DMSO-d₆) δ ppm 8.17 (br. s., 1H), 7.62-7.72 (m, 2H), 7.55 (br. s., 1H), 7.51 (d, J=6.60 Hz, 1H), 6.81 (br. s., 2H), 3.71 (s, 3H), 3.49 (br. s., 2H), 3.43 (br. s., 2H), 2.95 (t, J=5.56 Hz, 2H), 2.35-2.43 (m, 2H), 2.28 (s, 3H), 2.15 (br. s., 3H), 1.03 (br. s., 3H); LCMS: [M+H]⁺=468.16.

### Example 304: 6-(3-amino-6-(3-(1-(dimethylamino)ethyl)-4-hydroxyphenyl)-5-fluoropyrazin-2-yl)-4-methylisoquinolin-1(2H)-one (I-304)

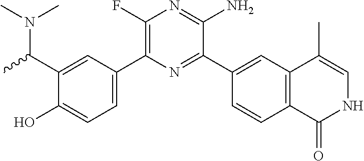 [SMILES: Cc1c[nH]c(=O)c2ccc(-c3nc(-c4ccc(O)c(C(C)N(C)C)c4)c(F)nc3N)cc12]

Prepared from 6-(3-amino-6-bromo-5-fluoropyrazin-2-yl)-4-methylisoquinolin-1(2H)-one (49.5 mg, 0.142 mmol) and 2-(1-(dimethylamino)ethyl)-4-(4,4,5,5-tetramethyl-1,3,2-dioxaborolan-2-yl)phenol (55 mg, 0.170 mmol) to give the title compound as a pale-yellow solid. (26 mg, 40% yield). ¹H NMR (500 MHz, DMSO-d₆) δ ppm 11.06-11.24 (m, 1H), 8.34 (d, J=8.31 Hz, 1H), 7.99 (s, 1H), 7.84 (d, J=8.19 Hz, 1H), 7.63-7.71 (m, 2H), 7.07 (d, J=4.89 Hz, 1H), 6.67-6.93 (m, 3H), 3.75 (br. s., 1H), 2.28 (d, J=6.11 Hz, 9H), 1.34 (d, J=6.60 Hz, 3H); LCMS: [M+H]⁺=434.32.

### Example 305: 6-(3-amino-6-(3-((dimethylamino)methyl)-4-hydroxyphenyl)-5-fluoropyrazin-2-yl)-4-fluoroisoquinolin-1(2H)-one (I-305)

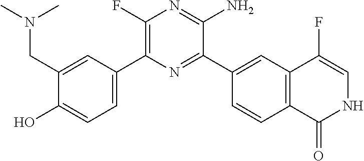 [SMILES: CN(C)Cc1cc(-c2nc(-c3ccc4c(=O)[nH]cc(F)c4c3)c(N)nc2F)ccc1O]

Prepared from 6-(3-amino-6-bromo-5-fluoropyrazin-2-yl)-4-fluoroisoquinolin-1(2H)-one (47.8 mg, 0.135 mmol) and 2-((dimethylamino)methyl)-4-(4,4,5,5-tetramethyl-1,3,2-dioxaborolan-2-yl)phenol (50 mg, 0.162 mmol, in turn prepared from a sequence starting from bromosalicylaldehyde and dimethylamine) to give the title compound was isolated as a pale-yellow solid. (22 mg, 36.5% yield). ¹H NMR (500 MHz, DMSO-d₆) δ 11.22 (br. s., 1H), 8.25-8.41 (m, 1H), 8.05 (s, 1H), 7.95 (d, J=8.31 Hz, 1H), 7.66 (d, J=8.68 Hz, 1H), 7.63 (s, 1H), 7.45 (d, J=5.75 Hz, 1H), 6.75-6.94 (m, 3H), 3.65 (s, 2H), 2.27 (s, 6H); LCMS: [M+H]⁺=424.29.

### Example 306: 6-(3-amino-6-(3-((dimethylamino)methyl)-4-hydroxyphenyl)-5-fluoropyrazin-2-yl)-4-methylisoquinolin-1(2H)-one (I-306)

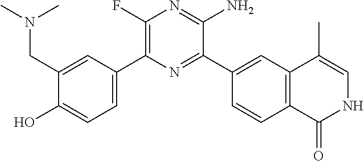 [SMILES: Cc1c[nH]c(=O)c2ccc(-c3nc(-c4ccc(O)c(CN(C)C)c4)c(F)nc3N)cc12]

Prepared from 6-(3-amino-6-bromo-5-fluoropyrazin-2-yl)-4-methylisoquinolin-1(2H)-one (47.2 mg, 0.135 mmol) and 2-((dimethylamino)methyl)-4-(4,4,5,5-tetramethyl-1,3,2-dioxaborolan-2-yl)phenol to give the title compound was isolated as a pale-yellow solid. (26 mg, 43.5% yield); ¹H NMR (500 MHz, DMSO-d₆) δ 11.15 (d, J=5.01 Hz, 1H), 8.28-8.40 (m, 1H), 7.98 (s, 1H), 7.81-7.89 (m, 1H), 7.71 (br. s., 2H), 7.08 (d, J=4.52 Hz, 1H), 6.90 (d, J=8.44 Hz, 1H), 6.78-6.87 (m, 2H), 3.82 (br. s., 2H), 2.32-2.45 (m, 6H), 2.27 (s, 3H); LCMS: [M+H]+=420.27.

### B: Preparation of Comparative Compounds

### Example C-1: 6-(3-amino-6-(4-morpholinophenyl)pyrazin-2-yl)-3,4-dihydroisoquinolin-1(2H)-one

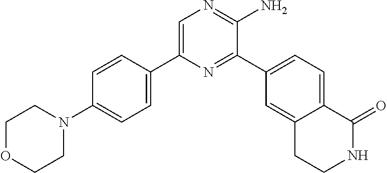 [SMILES: Nc1ncc(-c2ccc(N3CCOCC3)cc2)nc1-c1ccc2c(c1)CCNC2=O]

### Step 1: 3-chloro-5-(4-morpholinophenyl)pyrazin-2-amine

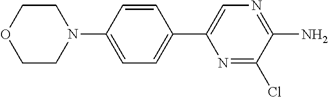 [SMILES: Nc1ncc(-c2ccc(N3CCOCC3)cc2)nc1Cl]

A vial charged with 2-amino-5-bromo-3-chloropyrazine (150 mg, 0.720 mmol), Cs₂CO₃ (703 mg, 2.16 mmol), 4-morpholinophenylboronic acid, pinacol ester (208 mg, 0.720 mmol) and XPhos Pd G2 (52.7 mg, 0.072 mmol) was suspended in water (2.5 mL) and DME (5 mL). The reaction was degassed by evacuation-refill with N₂ then heated sealed under microwave irradiation at 90° C. for 2 h. The reaction mixture was concentrated under reduced pressure, deposited on Celite® and purified by flash chromatography (12 g SiO₂ cartridge, eluting with 25% MeOH in CH₂Cl₂ eluting at 25% MeOH) and filtering fractions with product through a Waters PoraPak™ CX column (2 g), rinsing with MeOH and eluting with 2 M NH₃ in MeOH. The relevant fractions were concentrated, repurified by flash chromatography (25 g SiO₂ column, eluting with 8% MeOH in CH₂Cl₂) to afford the product (95 mg, 24%). LCMS: [M+H]⁺=291.26.

### Step 2: 6-(3-amino-6-(4-morpholinophenyl)pyrazin-2-yl)-3,4-dihydroisoquinolin-1(2H)-one, Trifluoroacetic Acid

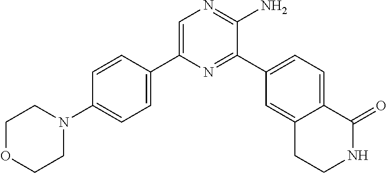 [SMILES: Nc1ncc(-c2ccc(N3CCOCC3)cc2)nc1-c1ccc2c(c1)CCNC2=O]

3-Chloro-5-(4-morpholinophenyl)pyrazin-2-amine (50 mg, 0.089 mmol), K₃PO₄ (95 mg, 0.447 mmol), XPhos Pd G2 (7.04 mg, 8.94 μmol), were placed under N₂. A solution of 6-(4,4,5,5-tetramethyl-1,3,2-dioxaborolan-2-yl)-3,4-dihydroisoquinolin-1(2H)-one (42.6 mg, 0.156 mmol) in 1,4-dioxane (1.5 mL), water (2 mL) and CH₃CN (3 mL) was added. The mixture was degassed with N₂ and then heated in a microwave reactor at 100° C. for 4 h. The reaction mixture was concentrated under reduced pressure, deposited on Celite® and purified by flash chromatography (25 g SiO₂ cartridge, eluting with 6% MeOH in CH₂Cl₂). Relevant fractions were collected, concentrated and repurified by preparative HPLC (30 g Biotage® SNAP KP-C₁₈-HS, 57% MeOH in (H₂O+0.05% TFA). The relevant fractions were combined and concentrated to afford 6-(3-amino-6-(4-morpholinophenyl)pyrazin-2-yl)-3,4-dihydroisoquinolin-1(2H)-one-TFA as an orange solid (7 mg, 15% based on purity of 97%). ¹H NMR (500 MHz, METHANOL-d₄) δ 8.33 (s, 1H), 8.10 (d, J=7.8 Hz, 1H), 7.94 (br d, J=7.9 Hz, 2H), 7.83-7.77 (m, 1H), 7.75 (s, 1H), 7.17 (br d, J=8.1 Hz, 2H), 3.89 (br s, 4H), 3.57 (br t, J=6.4 Hz, 2H), 3.36-3.31 (m, 4H), 3.10 (br t, J=6.5 Hz, 2H); LCMS: [M+H]⁺=402.39.

### C. Biological Assays

### (a) HPK1 Human STE Kinase Enzymatic Radiometric Assay

A stock solution of 10 mM of test compound is prepared in DMSO. The compound plate was prepared by 3-fold and 9-point serial dilutions. Recombinant (1-346) HPK1 (h) is incubated with 8 mM MOPS pH 7.0, 0.2 mM EDTA, 0.33 mg/mL myelin basic protein, 10 mM MgAcetate and [gamma-33P]-ATP (specific activity and concentration as required). The reaction is initiated by the addition of the Mg/ATP mix. After incubation for 40 minutes at RT, the reaction is stopped by the addition of phosphoric acid to a concentration of 0.5%, 10 μL of the reaction is then spotted onto a P30 filtermat and washed four times for 4 minutes in 0.425% phosphoric acid and once in methanol prior to drying and scintillation counting. For more details of kinase assay protocols, see: Gao, Y. et. al.; Biochem J.451 (2): 313-328, 2013. Compounds of the application showed activity as inhibitors of HPK1 having IC₅₀'s in the following ranges: A: 0.1-10 nM; B: 11-100 nM; C: 101-1000 nM; D: >1000 nM. Specific ranges for exemplary compounds of Formula (I) are shown in Table 2.

### (b) Lck Human STE Kinase Enzymatic Radiometric Assay

Lck (h) is incubated with 50 mM Tris pH 7.5, 0.1 mM EGTA, 0.1 mM Na3VO4, 250 uM KVEKIGEGTYGVVYK (Cdc2 peptide), 10 mM MgAcetate and [gamma-33P]-ATP (specific activity and concentration as required). The reaction is initiated by the addition of the Mg/ATP mix. After incubation for 40 minutes at room temperature, the reaction is stopped by the addition of phosphoric acid to a concentration of 0.5%, 10 μL of the reaction is then spotted onto a P30 filtermat and washed four times for 4 minutes in 0.425% phosphoric acid and once in methanol prior to drying and scintillation counting. For more details on the kinase assay procedures see: Gao, Y. et. al.; Biochem J., 451 (2): 313-328, 2013. Generally, and advantageously, compounds of the application showed significantly less inhibition of Lck compared with inhibition of HPK1 (see Table 2 where IC₅₀'s are reported the following ranges: A: 0.1-10 nM; B: 11-100 nM; C: 101-1000 nM; D: >1000 nM for the compounds of Formula (I)).

In some embodiments the presence of a halo substituent on the amino pyridine ring provides compounds with improved selectivity for inhibition of HPK1 vs Lck (see, for example, Table 3 and Table 4).

### (c) Human Jurkat T Lymphocyte Anti-Proliferation Assay

Assay principle: Jurkat cells are incubated with various concentrations of test compounds for 72h, and cell proliferation/cytotoxicity is measured via detection of ATP production.

Literature: See Cree, I A et. al.; Toxicol In Vitro., 11 (5): 553-556, 1997 for additional information of ATP detection.

Assay Procedure: Jurkat cells (cultured in α-MEM media with 10% FBS) are seeded at 2000 cells/well (50 μL) in a 384-well well black culture plate (Perkin Elmer). Test compounds (in DMSO) are added to cells using the HP digital dispenser, and incubated at 37° C., 5% CO₂ for 72 hours. ATP production is measured by adding 40 μL/well of ATPLite-1-Step reagent (Perkin Elmer), incubating for 5 min at RT with shaking followed by detection of luminescent signal using an Envision plate reader (Perkin Elmer). Data is normalized to untreated cells, and plotted using XLFit. IC₅₀ values are calculated using a 4 parameter dose-response equation by fitting the curve of % inhibition versus Log of compound concentration.

Results: In an embodiment, exemplary compounds of the application had IC₅₀'s in the range of 1.0 to >10 uM in this assay. In an embodiment, exemplary compounds of the application had IC₅₀'s in the range of 3.4 to >10 μM in this assay.

### (d) Human Jurkat T Lymphocyte IL-2 Release Assay

Assay principle: Stimulation of TCR signaling via anti-CD3/CD28 antibody treatment in Jurkat T cells will lead to IL-2 secretion into the culture media, detected by a luminescent energy transfer bead immunoassay (IL-2 AlphaLISA kit).

Literature: See Cauchon, E. et. al.; Analytical Biochemistry., 388 (1): 134-139, 2009 for additional information on AlphaLISA detection.

Assay procedure: Jurkat cells (cultured in α-MEM media with 10% FBS) are seeded at 0.2×10⁶ cells/well (100 μL) in a 96-well round bottom culture plate (Greiner). Test compounds (in DMSO) are added to cells using an HP digital dispenser, and incubated for 15 mins at RT. Cells are stimulated with 15 μL/mL (v/v) of soluble CD3/CD28 antibodies in α-MEM media (Stem Cell Technologies; 50 μL/well, 150 μL final assay volume), and incubated at 37° C., 5% CO₂ for 4 hours. Cells are centrifuged at 1500 rpm for 5 mins at RT, and 5 μL of culture media is transferred to a 96 well % area white plate (Perkin Elmer). IL-2 is detected by adding 20 μL/well of a mixture of both Acceptor beads and Biotin anti-IL-2 antibody (1:200 dilution of each), and incubated for 1 hour at RT with shaking. 25 μL/well of Donor beads (1:63 dilution) are then added and incubated in the dark for 30 mins at RT with shaking, followed by detection of luminescent signal using an Envision plate reader (Perkin Elmer). Data is normalized to untreated/stimulated cells, and plotted using XLFit. EC₅₀ values are calculated using a 4 parameter dose-response equation by fitting the curve of % stimulation versus Log of compound concentration.

Results: In an embodiment, exemplary compounds of the application had EC₁₅₀'s in the range of 0.01 to 5 μM using this assay. In an embodiment, exemplary compounds of the application had EC₁₅₀'s in the range of 0.04 to 1.14 μM using this assay.

(e) p-SLP76 S376 Phosphorylation Inhibition Assay

Assay principle: Stimulation of TCR signaling via anti-CD3/CD28 antibody treatment in Jurkat T cells will lead to phosphorylation of HPK1 at Serine 376, detected by a luminescent energy transfer bead immunoassay (p-SPL76 AlphaLISA kit).

Literature: See Cauchon, E. et. al.; Analytical Biochemistry., 388 (1): 134-139, 2009 for additional information on AlphaLISA detection.

Assay procedure: Jurkat cells (cultured in α-MEM media with 10% FBS) are collected and centrifuged, and seeded in a 96-well % area white plate (Perkin Elmer) at 0.1×10⁶ cells/well in HBSS (25 μL). Test compounds (in DMSO) are added to cells using an HP digital dispenser, and incubated for 15 mins at RT. Cells are stimulated with 15 μl/mL (v/v) of soluble CD3/CD28 antibodies in HBSS (Stem Cell Technologies; 25 μL, 50 μL final assay volume), and incubated at 37° C., 5% CO₂ for 1 hour. Cells are lysed with 10 μL/well of lysis buffer followed by incubation at RT for 10 mins with shaking. p-SLP76 S376 is detected by adding 15 μl/well of Acceptor beads (1:50 dilution), and incubated for 1 hour at RT with shaking. 15 μL/well of Donor beads (1:50 dilution) are then added and incubated at RT in the dark for 1 hour with shaking, followed by detection of luminescent signal using an Envision plate reader (Perkin Elmer). Data is normalized to untreated/stimulated cells, and plotted using Graph Pad Prism. IC₅₀ values are calculated using a 4 parameter dose-response equation by fitting the curve of % inhibition versus Log of compound concentration.

### f) Glucose Kinase (Glk) Assay

GLK(h) (MAP4K3(h)) is incubated with 8 mM MOPS pH 7.0, 0.2 mM EDTA, 250 μM RLGRDKYKTLRQIRQ, 10 mM Magnesium acetate and [9-33P-ATP] (specific activity and concentration as required). The reaction is initiated by the addition of the Mg/ATP mix. After incubation for 40 minutes at room temperature, the reaction is stopped by the addition of phosphoric acid to a concentration of 0.5%, 10 μl of the reaction is then spotted onto a P30 filtermat and washed four times for 4 minutes in 0.425% phosphoric acid and once in methanol prior to drying and scintillation counting. For more details on the kinase assay procedures see: Gao, Y. et. al.; Biochem J., 451 (2): 313-328, 2013.

Results: In an embodiment, exemplary compounds of the application had IC₅₀'s in the range of greater than 30× greater than the value for HPK1 in this assay. In an embodiment, exemplary compounds of the application had IC₅₀'s in the range of at least 70× greater than HPK1. in this assay.

While the present application has been described with reference to what are presently considered to be the preferred examples, it is to be understood that the application is not limited to the disclosed examples. To the contrary, the present application is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety. Where a term in the present application is found to be defined differently in a document incorporated herein by reference, the definition provided herein is to serve as the definition for the term.

TABLE 2

| | | | *HP | |
|---|---|---|---|---|
| | | | K1 | Lck |
| | | | IC₅₀ | IC₅₀ |
| Compound | | | nM | nM |
| I.D | IUPAC Chemical Name | Structures | Range | Range* |
| I-1 | 6-(3-amino-5-fluoro-6-(4- (4-isopropylpiperazin-1- yl)phenyl)pyrazin-2-yl)- 3,4-dihydroisoquinolin- 1(2H)-one | 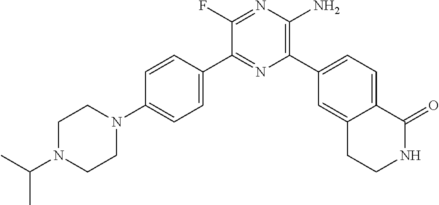 [SMILES: CC(C)N1CCN(c2ccc(-c3nc(-c4ccc5c(c4)CCNC5=O)c(N)nc3F)cc2)CC1] | A | D |
| I-2 | 6-(3-amino-5-fluoro-6-(4- (4-isopropylpiperazin-1- yl)phenyl)pyrazin-2-yl)-7- fluoro-3,4- dihydroisoquinolin-1(2H)- one | 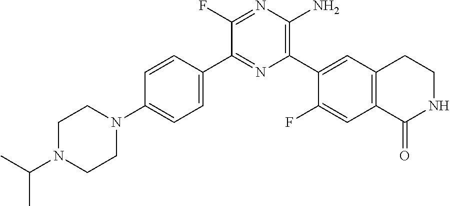 [SMILES: CC(C)N1CCN(c2ccc(-c3nc(-c4cc5c(cc4F)C(=O)NCC5)c(N)nc3F)cc2)CC1] | A | D |
| I-3 | 6-(3-amino-5-fluoro-6-(4- ((1R,5S)-3-methyl-3- azabicyclo[3.1.0]hexan-1- yl)phenyl)pyrazin-2-yl)-7- fluoro-3,4- dihydroisoquinolin-1(2H)- one | 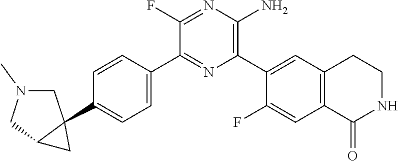 [SMILES: CN1C[C@H]2C[C@@]2(c2ccc(-c3nc(-c4cc5c(cc4F)C(=O)NCC5)c(N)nc3F)cc2)C1] | A | D |
| I-4 | 6-(3-amino-5-fluoro-6-(4- ((1R,5S)-3-isopropyl-3- azabicyclo[3.1.0]hexan-1- yl)phenyl)pyrazin-2-yl)-7- fluoro-3,4- dihydroisoquinolin-1(2H)- one | 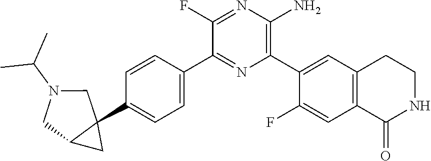 [SMILES: CC(C)N1C[C@H]2C[C@@]2(c2ccc(-c3nc(-c4cc5c(cc4F)C(=O)NCC5)c(N)nc3F)cc2)C1] | A | D |
| I-5 | 6-(3-amino-5-fluoro-6-(4- ((1S,5R)-3-methyl-3- azabicyclo[3.1.0]hexan-1- yl)phenyl)pyrazin-2-yl)-7- fluoro-3,4- dihydroisoquinolin-1(2H)- one | 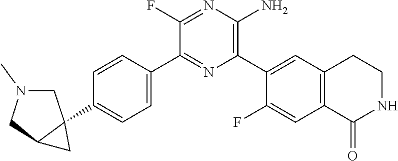 [SMILES: CN1C[C@@H]2C[C@]2(c2ccc(-c3nc(-c4cc5c(cc4F)C(=O)NCC5)c(N)nc3F)cc2)C1] | A | D |
| I-6 | 6-(3-amino-5-fluoro-6-(4- ((1S,5R)-3-isopropyl-3- azabicyclo[3.1.0]hexan-1- yl)phenyl)pyrazin-2-yl)-7- fluoro-3,4- dihydroisoquinolin-1(2H)- one | 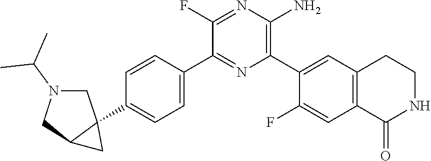 [SMILES: CC(C)N1C[C@@H]2C[C@]2(c2ccc(-c3nc(-c4cc5c(cc4F)C(=O)NCC5)c(N)nc3F)cc2)C1] | A | C |
| I-7 | 6-(3-amino-5-chloro-6-(4- (4-isopropylpiperazin-1- yl)phenyl)pyrazin-2-yl)- 3,4-dihydroisoquinolin- 1(2H)-one | 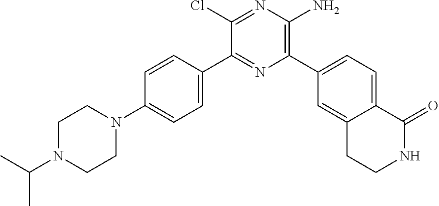 [SMILES: CC(C)N1CCN(c2ccc(-c3nc(-c4ccc5c(c4)CCNC5=O)c(N)nc3Cl)cc2)CC1] | B | D |
| I-8 | 6-(3-amino-5-chloro-6-(4- (4-isopropylpiperazin-1- yl)phenyl)pyrazin-2-yl)-7- fluoro-3,4- dihydroisoquinolin-1(2H)- one | 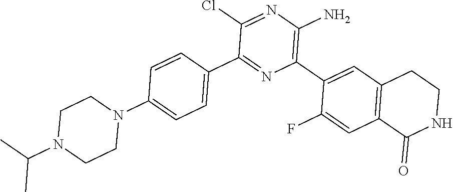 [SMILES: CC(C)N1CCN(c2ccc(-c3nc(-c4cc5c(cc4F)C(=O)NCC5)c(N)nc3Cl)cc2)CC1] | B | D |
| I-9 | 6-(3-amino-5-fluoro-6-(4- (1-isopropyl-1,2,3,6- tetrahydropyridin-4- yl)phenyl)pyrazin-2-yl)-7- fluoro-3,4- dihydroisoquinolin-1(2H)- one | 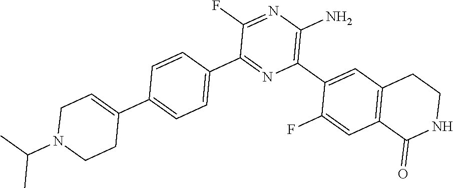 [SMILES: CC(C)N1CC=C(c2ccc(-c3nc(-c4cc5c(cc4F)C(=O)NCC5)c(N)nc3F)cc2)CC1] | A | C |
| I-10 | 6-(3-amino-5-fluoro-6-(4- (1-isopropylpiperidin-4- yl)phenyl)pyrazin-2-yl)-8- fluoro-3,4- dihydroisoquinolin-1(2H)- one | 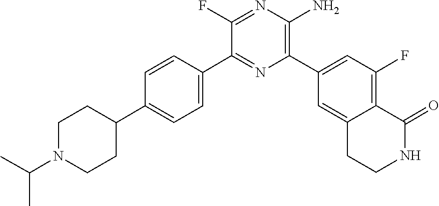 [SMILES: CC(C)N1CCC(c2ccc(-c3nc(-c4cc(F)c5c(c4)CCNC5=O)c(N)nc3F)cc2)CC1] | A | C |
| I-11 | 6-(3-amino-5-fluoro-6-(4- (4-isopropylpiperazin-1- yl)phenyl)pyrazin-2-yl)-3- ((methylamino)methyl)- 3,4-dihydroisoquinolin- 1(2H)-one | 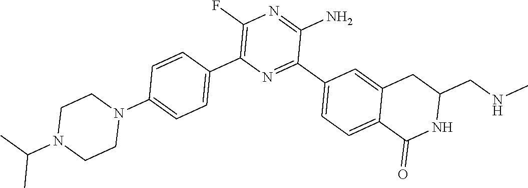 [SMILES: CNCC1Cc2cc(-c3nc(-c4ccc(N5CCN(C(C)C)CC5)cc4)c(F)nc3N)ccc2C(=O)N1] | A | D |
| I-12 | 6-(3-amino-5-fluoro-6-(4- morpholinophenyl) pyrazin-2-yl)-3- ((methylamino)methyl)- 3,4-dihydroisoquinolin- 1(2H)-one | 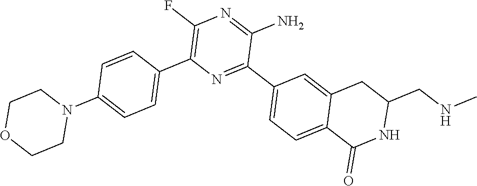 [SMILES: CNCC1Cc2cc(-c3nc(-c4ccc(N5CCOCC5)cc4)c(F)nc3N)ccc2C(=O)N1] | A | D |
| I-13 | 6-(3-amino-6-(4-(1- (cyclopropylmethyl) piperidin-4-yl)phenyl)-5- fluoropyrazin-2-yl)-8- fluoro-3,4- dihydroisoquinolin-1(2H)- one formate | 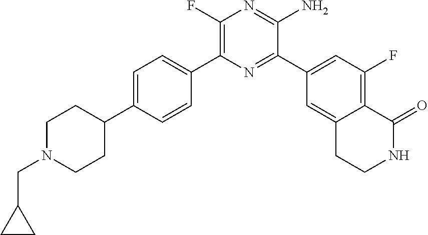 [SMILES: Nc1nc(F)c(-c2ccc(C3CCN(CC4CC4)CC3)cc2)nc1-c1cc(F)c2c(c1)CCNC2=O] | A | D |
| I-14 | 6-(3-amino-5-fluoro-6-(4- (4-isopropylpiperazin-1- yl)phenyl)pyrazin-2-yl)-8- fluoro-3,4- dihydroisoquinolin-1(2H)- one | 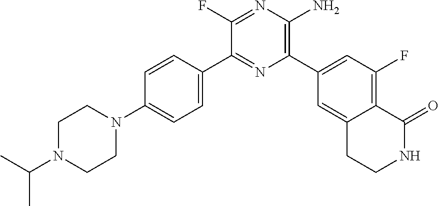 [SMILES: CC(C)N1CCN(c2ccc(-c3nc(-c4cc(F)c5c(c4)CCNC5=O)c(N)nc3F)cc2)CC1] | A | C |
| I-15 | 6-(3-amino-5-fluoro-6-(4- ((1R,5S)-3-isopropyl-3- azabicyclo[3.1.0]hexan-1- yl)phenyl)pyrazin-2-yl)-8- fluoro-3,4- dihydroisoquinolin-1(2H)- one formate | 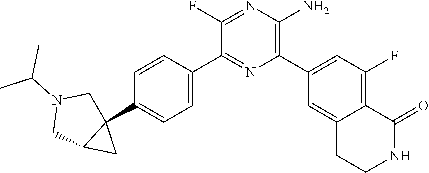 [SMILES: CC(C)N1C[C@H]2C[C@@]2(c2ccc(-c3nc(-c4cc(F)c5c(c4)CCNC5=O)c(N)nc3F)cc2)C1] | A | D |
| I-16 | 6-(3-amino-6-(4-(4- (cyclopropylmethyl) piperazin-1-yl)phenyl)-5- fluoropyrazin-2-yl)-8- fluoro-3,4- dihydroisoquinolin-1(2H)- one | 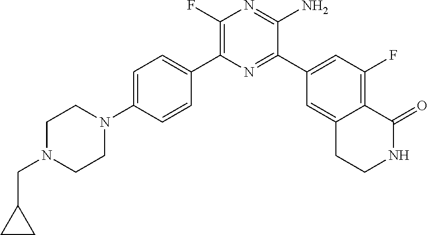 [SMILES: Nc1nc(F)c(-c2ccc(N3CCN(CC4CC4)CC3)cc2)nc1-c1cc(F)c2c(c1)CCNC2=O] | | |
| I-17 | 6-(3-amino-5-fluoro-6-(4- morpholinophenyl) pyrazin-2-yl)-3,4- dihydroisoquinolin-1(2H)- one | 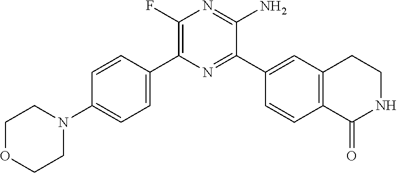 [SMILES: Nc1nc(F)c(-c2ccc(N3CCOCC3)cc2)nc1-c1ccc2c(c1)CCNC2=O] | A | D |
| I-18 | (S)-6-(3-amino-5-fluoro-6- (3-(pyrrolidin-2-yl)-4- (tetrahydro-2H-pyran-4- yl)phenyl)pyrazin-2-yl)- 3,4-dihydroisoquinolin- 1(2H)-one | 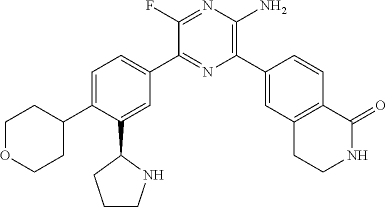 [SMILES: Nc1nc(F)c(-c2ccc(C3CCOCC3)c([C@@H]3CCCN3)c2)nc1-c1ccc2c(c1)CCNC2=O] | A | D |
| I-19 | (S)-6-(3-amino-5-fluoro-6- (3-(pyrrolidin-2-yl)-4- (tetrahydro-2H-pyran-4- yl)phenyl)pyrazin-2-yl)-7- fluoro-3,4- dihydroisoquinolin-1(2H)- one | 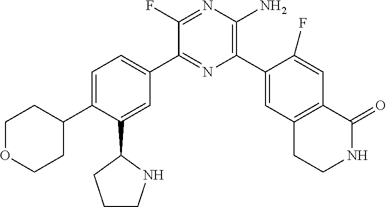 [SMILES: Nc1nc(F)c(-c2ccc(C3CCOCC3)c([C@@H]3CCCN3)c2)nc1-c1cc2c(cc1F)C(=O)NCC2] | A | |
| I-20 | 6-(3-amino-5-fluoro-6-(4- (4-isopropylpiperazin-1- yl)phenyl)pyrazin-2- yl)isoquinolin-1(2H)-one | 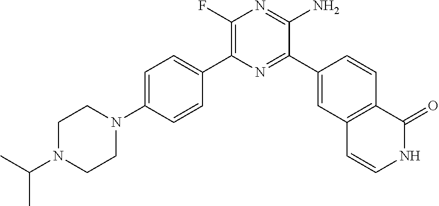 [SMILES: CC(C)N1CCN(c2ccc(-c3nc(-c4ccc5c(=O)[nH]ccc5c4)c(N)nc3F)cc2)CC1] | A | D |
| I-21 | 6-(3-amino-5-fluoro-6-(4- (4-isopropylpiperazin-1- yl)phenyl)pyrazin-2-yl)-8- fluoroisoquinolin-1(2H)- one | 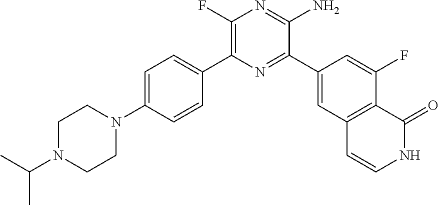 [SMILES: CC(C)N1CCN(c2ccc(-c3nc(-c4cc(F)c5c(=O)[nH]ccc5c4)c(N)nc3F)cc2)CC1] | A | C |
| I-22 | 6-(3-amino-5-fluoro-6-(4- (4-isopropylpiperazin-1- yl)phenyl)pyrazin-2-yl)-7- fluoroisoquinolin-1(2H)- one | 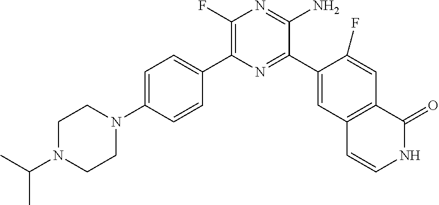 [SMILES: CC(C)N1CCN(c2ccc(-c3nc(-c4cc5cc[nH]c(=O)c5cc4F)c(N)nc3F)cc2)CC1] | A | C |
| I-23 | 6-(3-amino-6-(4-(4- (cyclopropylmethyl) piperazin-1-yl)phenyl)-5- fluoropyrazin-2- yl)isoquinolin-1(2H)-one | 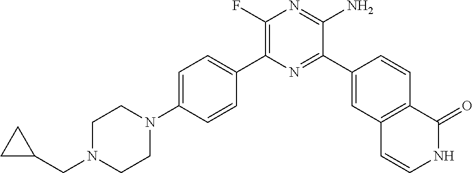 [SMILES: Nc1nc(F)c(-c2ccc(N3CCN(CC4CC4)CC3)cc2)nc1-c1ccc2c(=O)[nH]ccc2c1] | A | D |
| I-24 | 6-(3-amino-6-(4-(4- (cyclopropylmethyl) piperazin-1-yl)phenyl)-5- fluoropyrazin-2-yl)-8- fluoroisoquinolin-1(2H)- one | 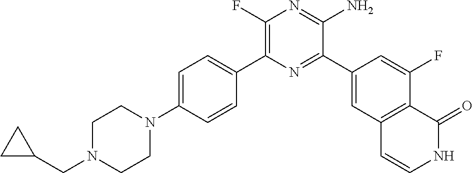 [SMILES: Nc1nc(F)c(-c2ccc(N3CCN(CC4CC4)CC3)cc2)nc1-c1cc(F)c2c(=O)[nH]ccc2c1] | A | C |
| I-25 | 6-(3-amino-6-(4-(4- (cyclopropylmethyl) piperazin-1-yl)phenyl)-5- fluoropyrazin-2-yl)-7- fluoroisoquinolin-1(2H)- one | 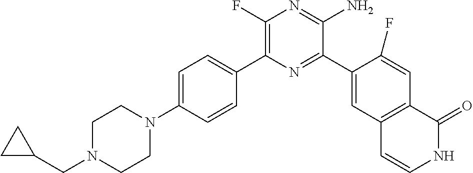 [SMILES: Nc1nc(F)c(-c2ccc(N3CCN(CC4CC4)CC3)cc2)nc1-c1cc2cc[nH]c(=O)c2cc1F] | A | C |
| I-26 | 6-(3-amino-6-(4-(4-(2- cyclopropylethyl)piperazin- 1-yl)phenyl)-5- fluoropyrazin-2-yl)-8- fluoro-3,4- dihydroisoquinolin-1(2H)- one | 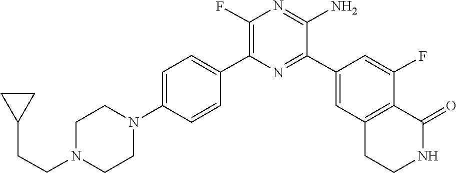 [SMILES: Nc1nc(F)c(-c2ccc(N3CCN(CCC4CC4)CC3)cc2)nc1-c1cc(F)c2c(c1)CCNC2=O] | A | D |
| I-27 | 6-(3-amino-6-(4-(4-(2- cyclopropylethyl)piperazin- 1-yl)phenyl)-5- fluoropyrazin-2-yl)-8- fluoroisoquinolin-1(2H)- one | 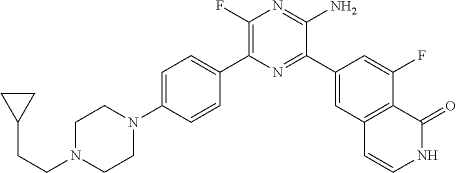 [SMILES: Nc1nc(F)c(-c2ccc(N3CCN(CCC4CC4)CC3)cc2)nc1-c1cc(F)c2c(=O)[nH]ccc2c1] | | |
| I-28 | 6-(3-amino-6-(4-(4-(2- cyclopropylethyl)piperazin- 1-yl)phenyl)-5- fluoropyrazin-2-yl)-7- fluoroisoquinolin-1(2H)- one | 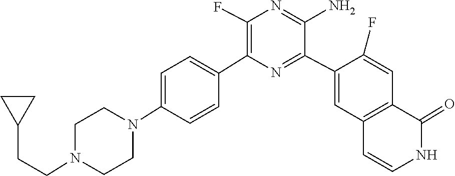 [SMILES: Nc1nc(F)c(-c2ccc(N3CCN(CCC4CC4)CC3)cc2)nc1-c1cc2cc[nH]c(=O)c2cc1F] | | |
| I-29 | (R)-6-(3-amino-6-(4-(4-(1- cyclopropylethyl)piperazin- 1-yl)phenyl)-5- fluoropyrazin-2-yl)-8- fluoro-3,4- dihydroisoquinolin-1(2H)- one, Enantiomer 1 | 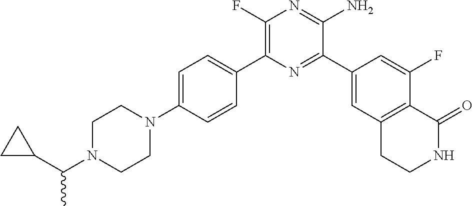 [SMILES: CC(C1CC1)N1CCN(c2ccc(-c3nc(-c4cc(F)c5c(c4)CCNC5=O)c(N)nc3F)cc2)CC1] | A | C |
| I-30 | (S)-6-(3-amino-6-(4-(4-(1- cyclopropylethyl)piperazin- 1-yl)phenyl)-5- fluoropyrazin-2-yl)-8- fluoro-3,4- dihydroisoquinolin-1(2H)- one, Enantiomer 2 | 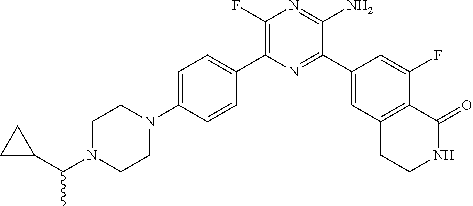 [SMILES: CC(C1CC1)N1CCN(c2ccc(-c3nc(-c4cc(F)c5c(c4)CCNC5=O)c(N)nc3F)cc2)CC1] | A | C |
| I-31 | 6-(3-amino-6-(4-(4- ethylpiperazin-1- yl)phenyl)-5- fluoropyrazin-2-yl)-8- fluoroisoquinolin-1(2H)- one | 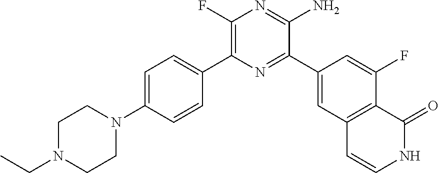 [SMILES: CCN1CCN(c2ccc(-c3nc(-c4cc(F)c5c(=O)[nH]ccc5c4)c(N)nc3F)cc2)CC1] | | |
| I-32 | 6-(3-amino-6-(4-(4- ethylpiperazin-1- yl)phenyl)-5- fluoropyrazin-2- yl)isoquinolin-1(2H)-one | 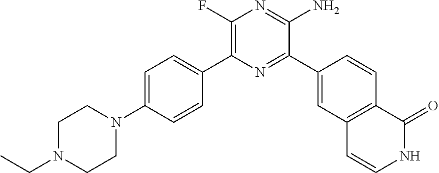 [SMILES: CCN1CCN(c2ccc(-c3nc(-c4ccc5c(=O)[nH]ccc5c4)c(N)nc3F)cc2)CC1] | A | D |
| I-33 | 6-(3-amino-5-fluoro-6-(4- morpholinophenyl)pyrazin- 2-yl)-3,4- dihydroisoquinolin-1(2H)- one | 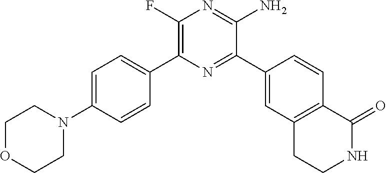 [SMILES: Nc1nc(F)c(-c2ccc(N3CCOCC3)cc2)nc1-c1ccc2c(c1)CCNC2=O] | A | D |
| I-34 | 6-(3-amino-5-fluoro-6-(4- morpholinophenyl)pyrazin- 2-yl)-7-fluoro-3,4- dihydroisoquinolin-1(2H)- one | 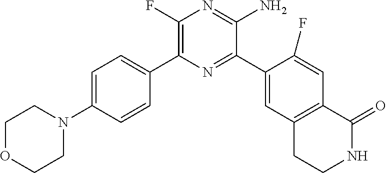 [SMILES: Nc1nc(F)c(-c2ccc(N3CCOCC3)cc2)nc1-c1cc2c(cc1F)C(=O)NCC2] | A | |
| I-35 | 6-(3-amino-5-fluoro-6-(4- morpholinophenyl)pyrazin- 2-yl)-8-fluoro-3,4- dihydroisoquinolin-1(2H)- one | 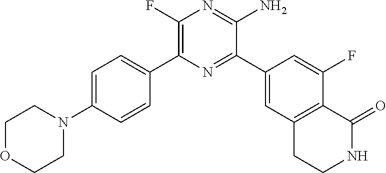 [SMILES: Nc1nc(F)c(-c2ccc(N3CCOCC3)cc2)nc1-c1cc(F)c2c(c1)CCNC2=O] | | |
| I-36 | 6-(3-amino-5-fluoro-6-(4- morpholinophenyl)pyrazin- 2-yl)-8-fluoroisoquinolin- 1(2H)-one | 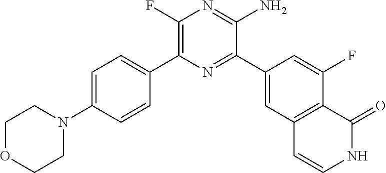 [SMILES: Nc1nc(F)c(-c2ccc(N3CCOCC3)cc2)nc1-c1cc(F)c2c(=O)[nH]ccc2c1] | A | D |
| I-37 | 7-(3-amino-5-fluoro-6-(4- (4-isopropylpiperazin-1- yl)phenyl)pyrazin-2- yl)quinazolin-4(3H)-one | 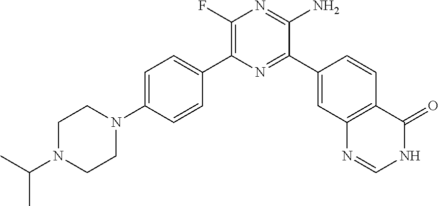 [SMILES: CC(C)N1CCN(c2ccc(-c3nc(-c4ccc5c(=O)[nH]cnc5c4)c(N)nc3F)cc2)CC1] | A | D |
| I-38 | 6-(3-amino-5-fluoro-6-(4- (4-isopropylpiperazin-1- yl)phenyl)pyrazin-2- yl)phthalazin-1(2H)-one | 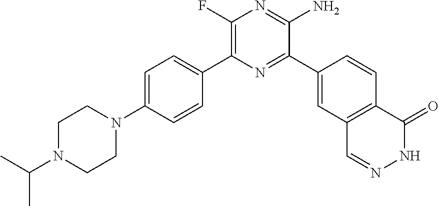 [SMILES: CC(C)N1CCN(c2ccc(-c3nc(-c4ccc5c(=O)[nH]ncc5c4)c(N)nc3F)cc2)CC1] | B | D |
| I-39 | 7-(3-amino-5-fluoro-6-(4- (4-isopropylpiperazin-1- yl)phenyl)pyrazin-2-yl)-2- methylquinazolin-4(3H)- one | 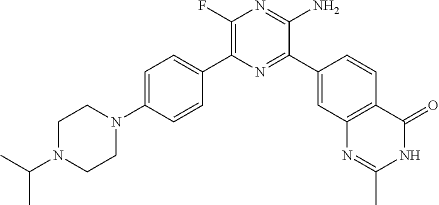 [SMILES: Cc1nc2cc(-c3nc(-c4ccc(N5CCN(C(C)C)CC5)cc4)c(F)nc3N)ccc2c(=O)[nH]1] | B | D |
| I-40 | 7-(3-amino-5-fluoro-6-(4- (4-isopropylpiperazin-1- yl)phenyl)pyrazin-2-yl)-5- fluoro-2-methylquinazolin- 4(3H)-one | 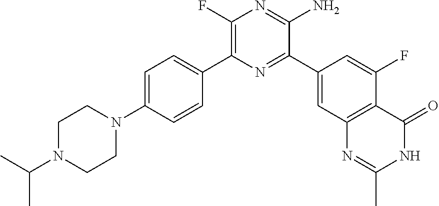 [SMILES: Cc1nc2cc(-c3nc(-c4ccc(N5CCN(C(C)C)CC5)cc4)c(F)nc3N)cc(F)c2c(=O)[nH]1] | | |
| I-41 | 6-(3-amino-5-fluoro-6- (4-(4-methylpiperazin- 1-yl)phenyl)pyrazin-2- yl)-3,4- dihydroisoquinolin- 1(2H)-one | 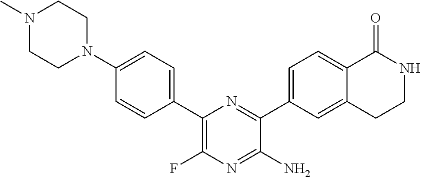 [SMILES: CN1CCN(c2ccc(-c3nc(-c4ccc5c(c4)CCNC5=O)c(N)nc3F)cc2)CC1] | A | |
| I-42 | 6-(3-amino-6-(3- ((dimethylamino)methyl)- 4-(tetrahydro-2H- pyran-4-yl)phenyl)-5- fluoropyrazin-2-yl)-4- fluoroisoquinolin-1(2H)- one | 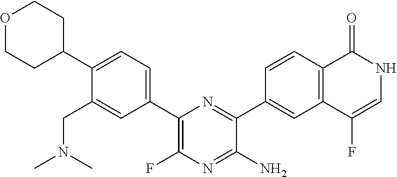 [SMILES: CN(C)Cc1cc(-c2nc(-c3ccc4c(=O)[nH]cc(F)c4c3)c(N)nc2F)ccc1C1CCOCC1] | A | D |
| I-43 | 6-(3-amino-5-fluoro-6- (4-(4- isopropylpiperazin-1- yl)phenyl)pyrazin-2-yl)- 4-chloro-3- methylisoquinolin- 1(2H)-one | 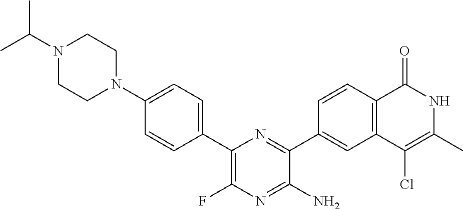 [SMILES: Cc1[nH]c(=O)c2ccc(-c3nc(-c4ccc(N5CCN(C(C)C)CC5)cc4)c(F)nc3N)cc2c1Cl] | A | |
| I-44 | 6-(3-amino-5-fluoro-6- (4-(4-(oxetan-3- yl)piperazin-1- yl)phenyl)pyrazin-2-yl)- 8-fluoro-3,4- dihydroisoquinolin- 1(2H)-one | 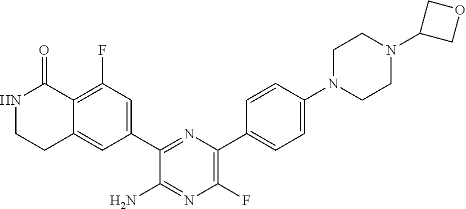 [SMILES: Nc1nc(F)c(-c2ccc(N3CCN(C4COC4)CC3)cc2)nc1-c1cc(F)c2c(c1)CCNC2=O] | A | D |
| I-45 | 6-(3-amino-5-fluoro-6- (4-(4-(oxetan-3- yl) piperazin-1- yl)phenyl)pyrazin-2-yl)- 7-fluoro-3,4- dihydroisoquinolin- 1(2H)-one | 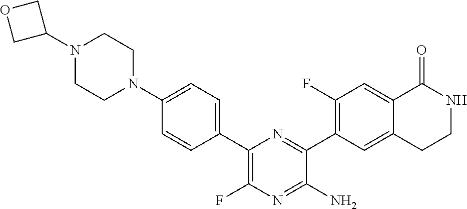 [SMILES: Nc1nc(F)c(-c2ccc(N3CCN(C4COC4)CC3)cc2)nc1-c1cc2c(cc1F)C(=O)NCC2] | B | |
| I-46 | 6-(3-amino-5-fluoro-6- (4-(4-(oxetan-3- yl)piperazin-1- yl)phenyl)pyrazin-2-yl)- 3,4-dihydroisoquinolin- 1(2H)-one | 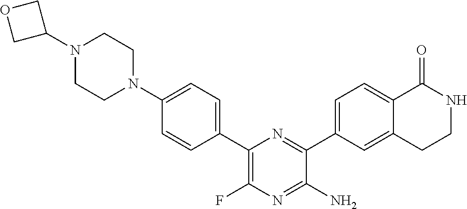 [SMILES: Nc1nc(F)c(-c2ccc(N3CCN(C4COC4)CC3)cc2)nc1-c1ccc2c(c1)CCNC2=O] | A | |
| I-47 | 6-(3-amino-6-(4-(4- cyclobutylpiperazin-1- yl)phenyl)-5- fluoropyrazin-2-yl)-8- fluoro-3,4- dihydroisoquinolin- 1(2H)-one | 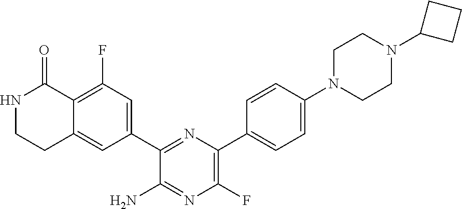 [SMILES: Nc1nc(F)c(-c2ccc(N3CCN(C4CCC4)CC3)cc2)nc1-c1cc(F)c2c(c1)CCNC2=O] | A | |
| I-48 | 6-(3-amino-6-(4-(4- cyclobutylpiperazin-1- yl)phenyl)-5- fluoropyrazin-2-yl)-7- fluoro-3,4- dihydroisoquinolin- 1(2H)-one | 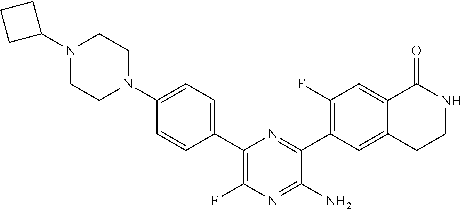 [SMILES: Nc1nc(F)c(-c2ccc(N3CCN(C4CCC4)CC3)cc2)nc1-c1cc2c(cc1F)C(=O)NCC2] | A | D |
| I-49 | 6-(3-amino-6-(4-(4- cyclobutylpiperazin-1- yl)phenyl)-5- fluoropyrazin-2-yl)-3,4- dihydroisoquinolin- 1(2H)-one | 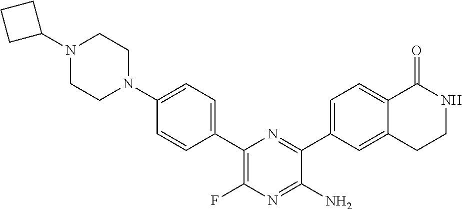 [SMILES: Nc1nc(F)c(-c2ccc(N3CCN(C4CCC4)CC3)cc2)nc1-c1ccc2c(c1)CCNC2=O] | A | |
| I-50 | (R)-6-(3-amino-5- fluoro-6-(4-(4-isopropyl- 2-methylpiperazin-1- yl)phenyl)pyrazin-2-yl)- 3,4-dihydroisoquinolin- 1(2H)-one | 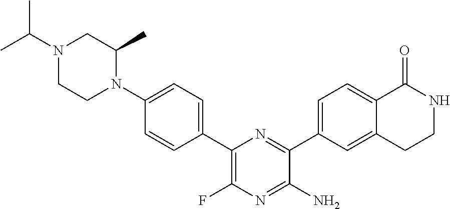 [SMILES: CC(C)N1CCN(c2ccc(-c3nc(-c4ccc5c(c4)CCNC5=O)c(N)nc3F)cc2)[C@H](C)C1] | A | |
| I-51 | (R)-6-(3-amino-5- fluoro-6-(4-(4-isopropyl- 2-methylpiperazin-1- yl)phenyl)pyrazin-2-yl)- 7-fluoro-3,4- dihydroisoquinolin- 1(2H)-one | 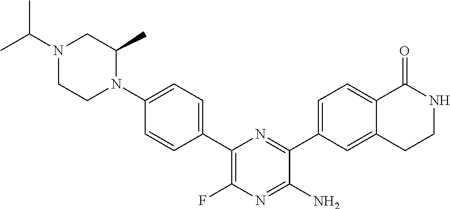 [SMILES: CC(C)N1CCN(c2ccc(-c3nc(-c4ccc5c(c4)CCNC5=O)c(N)nc3F)cc2)[C@H](C)C1] | A | |
| I-52 | (R)-6-(3-amino-6-(4- (2,4-dimethylpiperazin- 1-yl)phenyl)-5- fluoropyrazin-2-yl)-3,4- dihydroisoquinolin- 1(2H)-one | 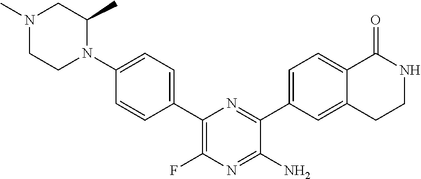 [SMILES: C[C@@H]1CN(C)CCN1c1ccc(-c2nc(-c3ccc4c(c3)CCNC4=O)c(N)nc2F)cc1] | A | |
| I-53 | (R)-6-(3-amino-6-(4- (2,4-dimethylpiperazin- 1-yl)phenyl)-5- fluoropyrazin-2-yl)-7- fluoro-3,4- dihydroisoquinolin- 1(2H)-one | 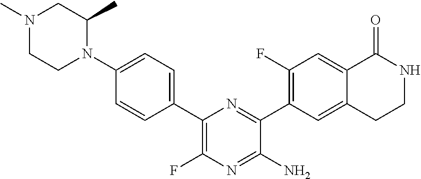 [SMILES: C[C@@H]1CN(C)CCN1c1ccc(-c2nc(-c3cc4c(cc3F)C(=O)NCC4)c(N)nc2F)cc1] | A | |
| I-54 | 6-(3-amino-5-fluoro-6- (4-(4-methylpiperazin- 1-yl)phenyl)pyrazin-2- yl)-7-fluoro-3,4- dihydroisoquinolin- 1(2H)-one | 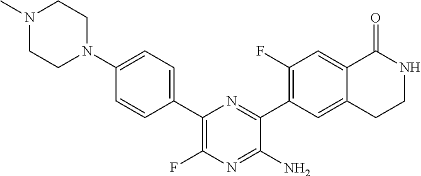 [SMILES: CN1CCN(c2ccc(-c3nc(-c4cc5c(cc4F)C(=O)NCC5)c(N)nc3F)cc2)CC1] | A | |
| I-55 | 6-(3-amino-6-(4-(4- ethylpiperazin-1- yl)phenyl)-5- fluoropyrazin-2-yl)-3,4- dihydroisoquinolin- 1(2H)-one | 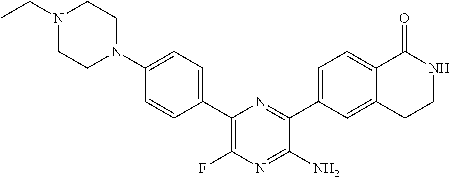 [SMILES: CCN1CCN(c2ccc(-c3nc(-c4ccc5c(c4)CCNC5=O)c(N)nc3F)cc2)CC1] | A | |
| I-56 | 6-(3-amino-6-(4-(4- ethylpiperazin-1- yl)phenyl)-5- fluoropyrazin-2-yl)-7- fluoro-3,4- dihydroisoquinolin- 1(2H)-one | 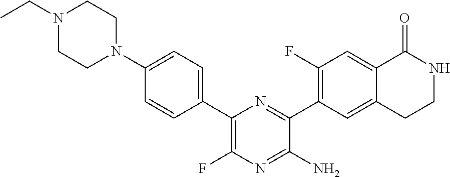 [SMILES: CCN1CCN(c2ccc(-c3nc(-c4cc5c(cc4F)C(=O)NCC5)c(N)nc3F)cc2)CC1] | A | |
| I-57 | 6-(3-amino-5-fluoro-6- (4-((3R,5S)-3,4,5- trimethylpiperazin-1- yl)phenyl)pyrazin-2-yl)- 3,4-dihydroisoquinolin- 1(2H)-one | 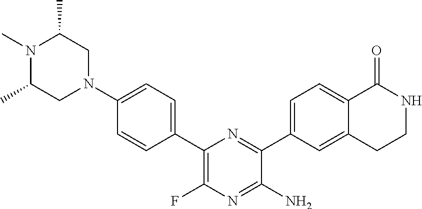 [SMILES: C[C@@H]1CN(c2ccc(-c3nc(-c4ccc5c(c4)CCNC5=O)c(N)nc3F)cc2)C[C@H](C)N1C] | A | |
| I-58 | 6-(3-amino-5-fluoro-6- (4-((3R,5S)-3,4,5- trimethylpiperazin-1- yl)phenyl)pyrazin-2-yl)- 7-fluoro-3,4- dihydroisoquinolin- 1(2H)-one | 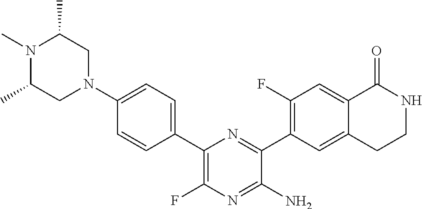 [SMILES: C[C@@H]1CN(c2ccc(-c3nc(-c4cc5c(cc4F)C(=O)NCC5)c(N)nc3F)cc2)C[C@H](C)N1C] | A | |
| I-59 | 6-(3-amino-6-(3- ((dimethylamino)methyl)- 4-(tetrahydro-2H- pyran-4-yl)phenyl)-5- fluoropyrazin-2-yl)-3,4- dihydroisoquinolin- 1(2H)-one | 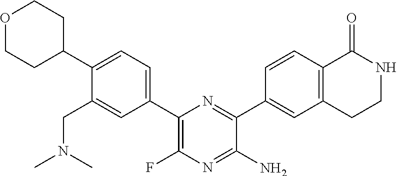 [SMILES: CN(C)Cc1cc(-c2nc(-c3ccc4c(c3)CCNC4=O)c(N)nc2F)ccc1C1CCOCC1] | A | D |
| I-60 | 6-(3-amino-6-(3- ((dimethylamino)methyl)- 4-(tetrahydro-2H- pyran-4-yl)phenyl)-5- fluoropyrazin-2-yl)-7- fluoro-3,4- dihydroisoquinolin- 1(2H)-one | 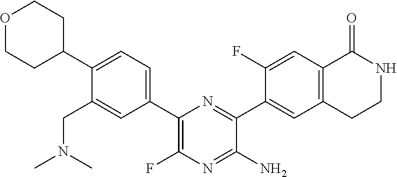 [SMILES: CN(C)Cc1cc(-c2nc(-c3cc4c(cc3F)C(=O)NCC4)c(N)nc2F)ccc1C1CCOCC1] | A | D |
| I-61 | 6-(3-amino-5-fluoro-6- (4-(2-methyl-2,7- diazaspiro[3.5]nonan-7- yl)phenyl)pyrazin-2-yl)- 7-fluoro-3,4- dihydroisoquinolin- 1(2H)-one | 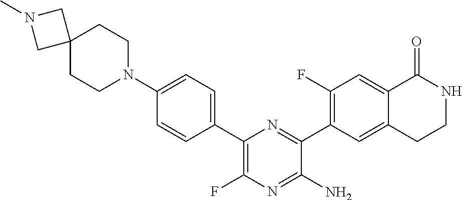 [SMILES: CN1CC2(CCN(c3ccc(-c4nc(-c5cc6c(cc5F)C(=O)NCC6)c(N)nc4F)cc3)CC2)C1] | A | |
| I-62 | 6-(3-amino-5-fluoro-6- (4-(4- isopropylpiperazin-1- yl)phenyl)pyrazin-2-yl)- 4-methylisoquinolin- 1(2H)-one | 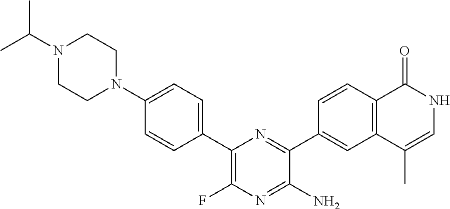 [SMILES: Cc1c[nH]c(=O)c2ccc(-c3nc(-c4ccc(N5CCN(C(C)C)CC5)cc4)c(F)nc3N)cc12] | A | C |
| I-63 | 7-(3-amino-5-fluoro-6- (4-(4- isopropylpiperazin-1- yl)phenyl)pyrazin-2-yl)- 6-fluoro-2- methylquinazolin- 4(3H)-one | 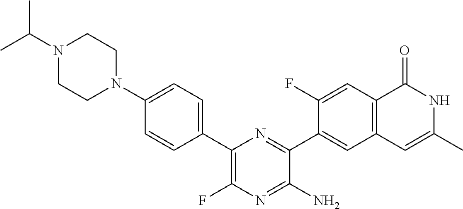 [SMILES: Cc1cc2cc(-c3nc(-c4ccc(N5CCN(C(C)C)CC5)cc4)c(F)nc3N)c(F)cc2c(=O)[nH]1] | A | |
| I-64 | 6-(3-amino-6-(3- ((dimethylamino)methyl)- 4-morpholinophenyl)- 5-fluoropyrazin-2-yl)- 3,4-dihydroisoquinolin- 1(2H)-one | 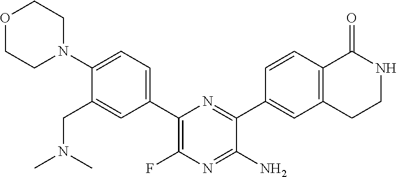 [SMILES: CN(C)Cc1cc(-c2nc(-c3ccc4c(c3)CCNC4=O)c(N)nc2F)ccc1N1CCOCC1] | A | D |
| I-65 | 6-(3-amino-6-(3- ((dimethylamino)methyl)- 4-morpholinophenyl)- 5-fluoropyrazin-2-yl)-7- fluoro-3,4- dihydroisoquinolin- 1(2H)-one | 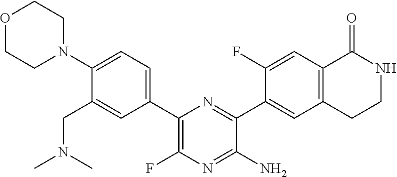 [SMILES: CN(C)Cc1cc(-c2nc(-c3cc4c(cc3F)C(=O)NCC4)c(N)nc2F)ccc1N1CCOCC1] | A | 0 |
| I-66 | 6-(3-amino-5-fluoro-6- (4-(4- isopropylpiperazin-1- yl)phenyl)pyrazin-2-yl)- 4-fluoroisoquinolin- 1(2H)-one | 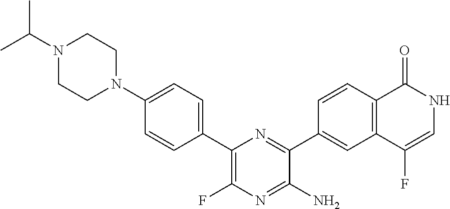 [SMILES: CC(C)N1CCN(c2ccc(-c3nc(-c4ccc5c(=O)[nH]cc(F)c5c4)c(N)nc3F)cc2)CC1] | A | D |
| I-67 | 6-(3-amino-5-fluoro-6- (4-(4- isopropylpiperazin-1- yl)phenyl)pyrazin-2-yl)- 8-fluoro-3- methylisoquinolin- 1(2H)-one | 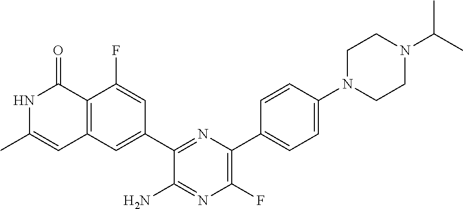 | A | D |
| I-68 | 6-(3-amino-6-(4-(4-(4,4- difluorobutyl)piperazin- 1-yl)phenyl)-5- fluoropyrazin-2-yl)-3,4- dihydroisoquinolin- 1(2H)-one | 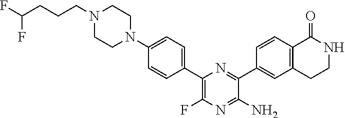 [SMILES: Nc1nc(F)c(-c2ccc(N3CCN(CCCC(F)F)CC3)cc2)nc1-c1ccc2c(c1)CCNC2=O] | A | |
| I-69 | 6-(3-amino-6-(4-(4-(4,4- difluorobutyl)piperazin- 1-yl)phenyl)-5- fluoropyrazin-2-yl)-7- fluoro-3,4- dihydroisoquinolin- 1(2H)-one | 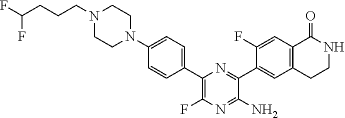 [SMILES: Nc1nc(F)c(-c2ccc(N3CCN(CCCC(F)F)CC3)cc2)nc1-c1cc2c(cc1F)C(=O)NCC2] | A | |
| I-70 | 6-(3-amino-6-(4-(4-(4,4- difluorobutyl)piperazin- 1-yl)phenyl)-5- fluoropyrazin-2- yl)isoquinolin-1(2H)- one | 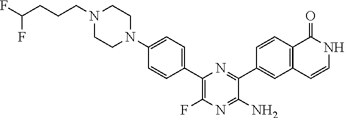 [SMILES: Nc1nc(F)c(-c2ccc(N3CCN(CCCC(F)F)CC3)cc2)nc1-c1ccc2c(=O)[nH]ccc2c1] | A | |
| I-71 | 6-(3-amino-5-fluoro-6- (4-(4- isopropylpiperazin-1- yl)phenyl)pyrazin-2-yl)- 4-fluoro-3- methylisoquinolin- 1(2H)-one | 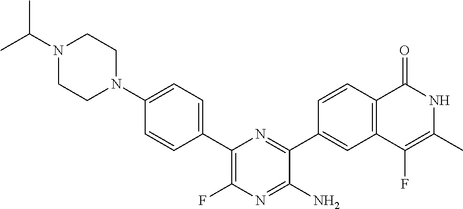 [SMILES: Cc1[nH]c(=O)c2ccc(-c3nc(-c4ccc(N5CCN(C(C)C)CC5)cc4)c(F)nc3N)cc2c1F] | A | D |
| I-72 | 6-(3-amino-6-(4-(4- (cyclopropylmethyl)pipe razin-1-yl)phenyl)-5- fluoropyrazin-2-yl)-4- methylisoquinolin- 1(2H)-one | 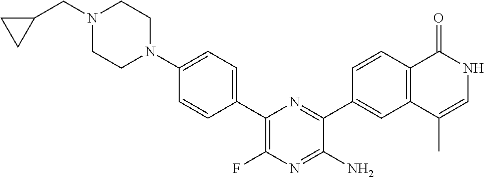 [SMILES: Cc1c[nH]c(=O)c2ccc(-c3nc(-c4ccc(N5CCN(CC6CC6)CC5)cc4)c(F)nc3N)cc12] | A | D |
| I-73 | 6-(3-amino-6-(4-(4-(4,4- difluorobutyl)piperazin- 1-yl)phenyl)-5- fluoropyrazin-2-yl)-4- methylisoquinolin- 1(2H)-one | 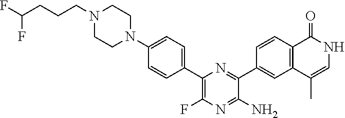 [SMILES: Cc1c[nH]c(=O)c2ccc(-c3nc(-c4ccc(N5CCN(CCCC(F)F)CC5)cc4)c(F)nc3N)cc12] | A | |
| I-74 | 7-(3-amino-5-fluoro-6- (4-((1S,5R)-3-methyl-3- azabicyclo[3.1.0]hexan- 1-yl)phenyl)pyrazin-2- yl)-2-methylquinazolin- 4(3H)-one | 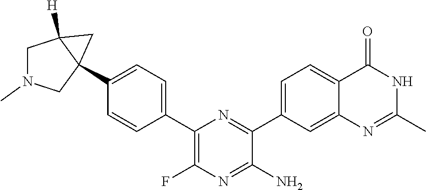 | B | |
| I-75 | 6-(3-amino-5-fluoro-6- (4-((1S,5R)-3-methyl-3- azabicyclo[3.1.0]hexan- 1-yl)phenyl)pyrazin-2- yl)-4-methylisoquinolin- 1(2H)-one | 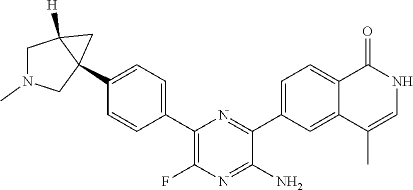 [SMILES: Cc1c[nH]c(=O)c2ccc(-c3nc(-c4ccc([C@]56C[C@H]5CN(C)C6)cc4)c(F)nc3N)cc12] | B | |
| I-76 | 7-(3-amino-6-(4-(4- cyclobutylpiperazin-1- yl)phenyl)-5- fluoropyrazin-2-yl)-2- methylquinazolin- 4(3H)-one | 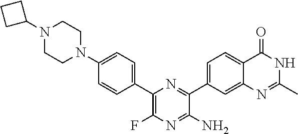 [SMILES: Cc1nc2cc(-c3nc(-c4ccc(N5CCN(C6CCC6)CC5)cc4)c(F)nc3N)ccc2c(=O)[nH]1] | B | |
| I-77 | 7-(3-amino-6-(3- ((dimethylamino)methyl)- 4-morpholinophenyl)- 5-fluoropyrazin-2-yl)-2- methylquinazolin- 4(3H)-one | 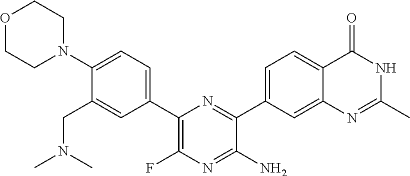 [SMILES: Cc1nc2cc(-c3nc(-c4ccc(N5CCOCC5)c(CN(C)C)c4)c(F)nc3N)ccc2c(=O)[nH]1] | B | |
| I-78 | 6-(3-amino-5-fluoro-6- (4-(4- isopropylpiperazin-1- yl)phenyl)pyrazin-2-yl)- 4-chloroisoquinolin- 1(2H)-one | 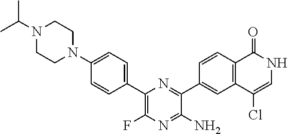 [SMILES: CC(C)N1CCN(c2ccc(-c3nc(-c4ccc5c(=O)[nH]cc(Cl)c5c4)c(N)nc3F)cc2)CC1] | A | C |
| I-79 | 7-(3-amino-6-(4-(4- (cyclopropylmethyl)pipe razin-1-yl)phenyl)-5- fluoropyrazin-2-yl)-2- methylquinazolin- 4(3H)-one | 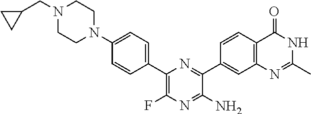 [SMILES: Cc1nc2cc(-c3nc(-c4ccc(N5CCN(CC6CC6)CC5)cc4)c(F)nc3N)ccc2c(=O)[nH]1] | C | |
| I-80 | 6-(3-amino-5-fluoro-6- (4-((1S,5R)-3-methyl-3- azabicyclo[3.1.0]hexan- 1-yl)phenyl)pyrazin-2- yl)-4-fluoroisoquinolin- 1(2H)-one | 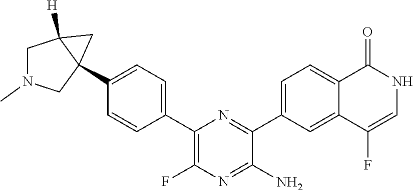 [SMILES: CN1C[C@@H]2C[C@]2(c2ccc(-c3nc(-c4ccc5c(=O)[nH]cc(F)c5c4)c(N)nc3F)cc2)C1] | A | D |
| I-81 | 7-(3-amino-6-(4-(4-(4,4- difluorobutyl)piperazin- 1-yl)phenyl)-5- fluoropyrazin-2-yl)-2- methylquinazolin- 4(3H)-one | 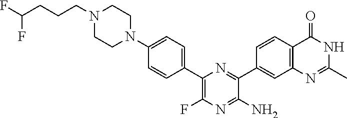 [SMILES: Cc1nc2cc(-c3nc(-c4ccc(N5CCN(CCCC(F)F)CC5)cc4)c(F)nc3N)ccc2c(=O)[nH]1] | B | |
| I-82 | 6-(3-amino-6-(4-(4-(3,3- difluoropropyl)piperazin- 1-yl)phenyl)-5- fluoropyrazin-2-yl)-8- fluoro-3,4- dihydroisoquinolin- 1(2H)-one | 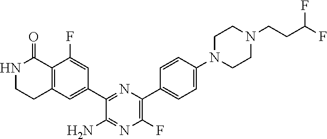 [SMILES: Nc1nc(F)c(-c2ccc(N3CCN(CCC(F)F)CC3)cc2)nc1-c1cc(F)c2c(c1)CCNC2=O] | A | |
| I-83 | 6-(3-amino-6-(3- ((dimethylamino)methyl)- 4-(tetrahydro-2H- pyran-4-yl)phenyl)-5- fluoropyrazin-2-yl)-8- fluoro-3,4- dihydroisoquinolin- 1(2H)-one | 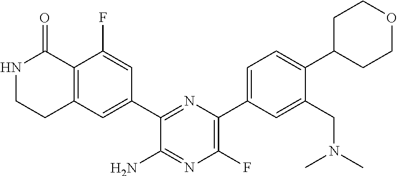 [SMILES: CN(C)Cc1cc(-c2nc(-c3cc(F)c4c(c3)CCNC4=O)c(N)nc2F)ccc1C1CCOCC1] | A | D |
| I-84 | 6-(3-amino-6-(4-(4- (cyclopropylmethyl)pipe razin-1-yl)phenyl)-5- fluoropyrazin-2-yl)-4- fluoroisoquinolin-1(2H)- one | 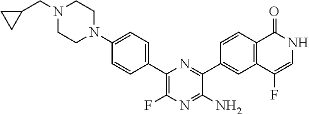 [SMILES: Nc1nc(F)c(-c2ccc(N3CCN(CC4CC4)CC3)cc2)nc1-c1ccc2c(=O)[nH]cc(F)c2c1] | A | |
| I-85 | 6-(3-amino-5-fluoro-6- (4-(4- isopropylpiperazin-1- yl)phenyl)pyrazin-2-yl)- 4,8-difluoroisoquinolin- 1(2H)-one | 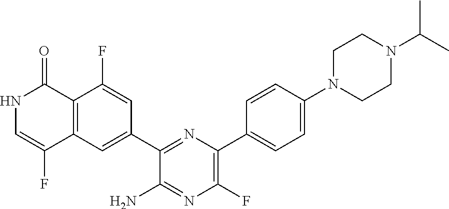 [SMILES: CC(C)N1CCN(c2ccc(-c3nc(-c4cc(F)c5c(=O)[nH]cc(F)c5c4)c(N)nc3F)cc2)CC1] | A | D |
| I-86 | 6-(3-amino-6-(4-(4-(3,3- difluoropropyl)piperazin- 1-yl)phenyl)-5- fluoropyrazin-2- yl)isoquinolin-1(2H)- one | 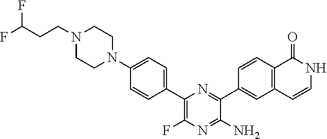 [SMILES: Nc1nc(F)c(-c2ccc(N3CCN(CCC(F)F)CC3)cc2)nc1-c1ccc2c(=O)[nH]ccc2c1] | B | |
| I-87 | 6-(3-amino-6-(4-(4-(3,3- difluoropropyl)piperazin- 1-yl)phenyl)-5- fluoropyrazin-2-yl)-4- methylisoquinolin- 1(2H)-one | 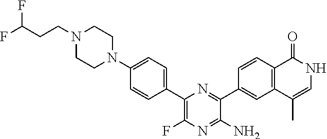 [SMILES: Cc1c[nH]c(=O)c2ccc(-c3nc(-c4ccc(N5CCN(CCC(F)F)CC5)cc4)c(F)nc3N)cc12] | D | |
| I-88 | 6-(3-amino-6-(3- ((dimethylamino)methyl)- 4-morpholinophenyl)- 5-fluoropyrazin-2-yl)-8- fluoro-3,4- dihydroisoquinolin- 1(2H)-one | 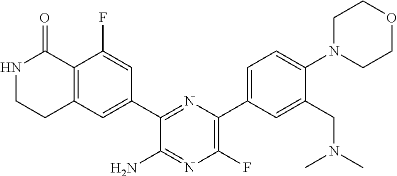 [SMILES: CN(C)Cc1cc(-c2nc(-c3cc(F)c4c(c3)CCNC4=O)c(N)nc2F)ccc1N1CCOCC1] | B | |
| I-89 | 6-(3-amino-6-(3- ((dimethylamino)methyl)- 4-morpholinophenyl)- 5-fluoropyrazin-2-yl)-4- fluoroisoquinolin-1(2H)- one | 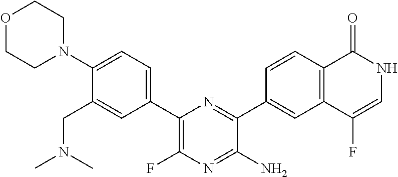 [SMILES: CN(C)Cc1cc(-c2nc(-c3ccc4c(=O)[nH]cc(F)c4c3)c(N)nc2F)ccc1N1CCOCC1] | A | |
| I-90 | 6-(3-amino-5-fluoro-6- (4-(4- isopropylpiperazin-1- yl)phenyl)pyrazin-2-yl)- 4-chloro-8- fluoroisoquinolin-1(2H)- one | 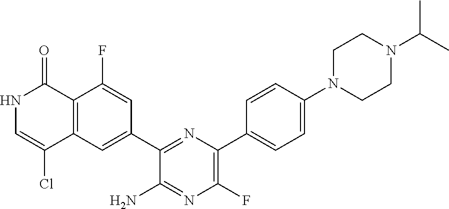 [SMILES: CC(C)N1CCN(c2ccc(-c3nc(-c4cc(F)c5c(=O)[nH]cc(Cl)c5c4)c(N)nc3F)cc2)CC1] | A | |
| I-91 | 7-(3-amino-6-(3- ((dimethylamino)methyl)- 4-(tetrahydro-2H- pyran-4-yl)phenyl)-5- fluoropyrazin-2-yl)-5- fluoro-2- methylquinazolin- 4(3H)-one | 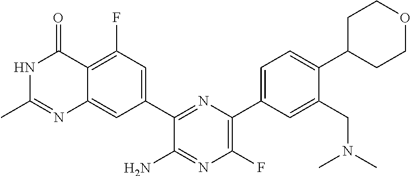 [SMILES: Cc1nc2cc(-c3nc(-c4ccc(C5CCOCC5)c(CN(C)C)c4)c(F)nc3N)cc(F)c2c(=O)[nH]1] | A | D |
| I-92 | 7-(3-amino-6-(3- ((dimethylamino)methyl)- 4-(tetrahydro-2H- pyran-4-yl)phenyl)-5- fluoropyrazin-2-yl)-2- methylquinazolin- 4(3H)-one | 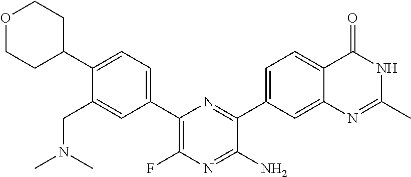 [SMILES: Cc1nc2cc(-c3nc(-c4ccc(C5CCOCC5)c(CN(C)C)c4)c(F)nc3N)ccc2c(=O)[nH]1] | A | D |
| I-93 | 6-(3-amino-5-fluoro-6- (4-((3R,5S)-3,4,5- trimethylpiperazin-1- yl)phenyl)pyrazin-2-yl)- 4-fluoroisoquinolin- 1(2H)-one | 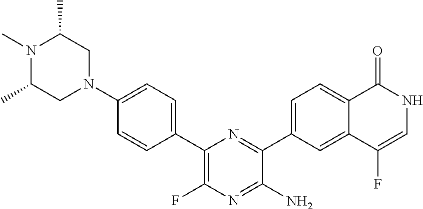 [SMILES: C[C@@H]1CN(c2ccc(-c3nc(-c4ccc5c(=O)[nH]cc(F)c5c4)c(N)nc3F)cc2)C[C@H](C)N1C] | A | |
| I-94 | 6-(3-amino-5-fluoro-6- (4-((3R,5S)-3,4,5- trimethylpiperazin-1- yl)phenyl)pyrazin-2-yl)- 4-methylisoquinolin- 1(2H)-one | 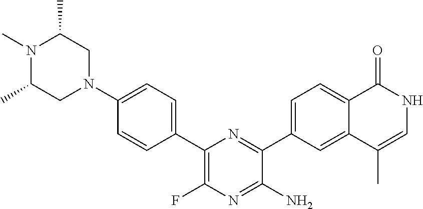 [SMILES: Cc1c[nH]c(=O)c2ccc(-c3nc(-c4ccc(N5C[C@@H](C)N(C)[C@@H](C)C5)cc4)c(F)nc3N)cc12] | | |
| I-95 | 7-(3-amino-5-fluoro-6- (4-((3R,5S)-3,4,5- trimethylpiperazin-1- yl)phenyl)pyrazin-2-yl)- 2-methylquinazolin- 4(3H)-one | 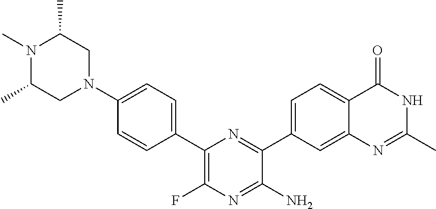 | B | |
| I-96 | 6-(3-amino-6-(3- ((dimethylamino)methyl)- 4-morpholinophenyl)- 5-fluoropyrazin-2-yl)-4- methylisoquinolin- 1(2H)-one | 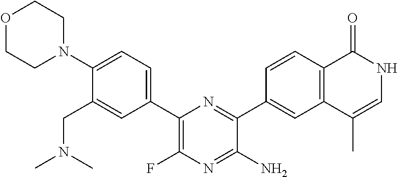 [SMILES: Cc1c[nH]c(=O)c2ccc(-c3nc(-c4ccc(N5CCOCC5)c(CN(C)C)c4)c(F)nc3N)cc12] | A | D |
| I-97 | 6-(3-amino-6-(3- ((dimethylamino)methyl)- 4-(tetrahydro-2H- pyran-4-yl)phenyl)-5- fluoropyrazin-2-yl)-4- fluoro-3- methylisoquinolin- 1(2H)-one | 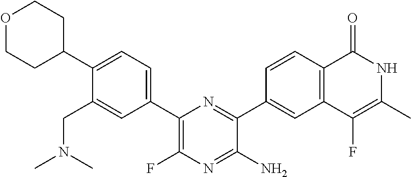 [SMILES: Cc1[nH]c(=O)c2ccc(-c3nc(-c4ccc(C5CCOCC5)c(CN(C)C)c4)c(F)nc3N)cc2c1F] | A | D |
| I-98 | 6-(3-amino-6-(3- ((dimethylamino)methyl)- 4-(tetrahydro-2H- pyran-4-yl)phenyl)-5- fluoropyrazin-2-yl)-4- methylisoquinolin- 1(2H)-one | 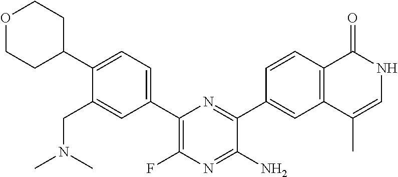 [SMILES: Cc1c[nH]c(=O)c2ccc(-c3nc(-c4ccc(C5CCOCC5)c(CN(C)C)c4)c(F)nc3N)cc12] | A | D |
| I-99 | 6-(3-amino-6-(4- ((1S,5R)-3-cyclobutyl-3 azabicyclo[3.1.0]hexan- 1-yl)phenyl)-5- fluoropyrazin-2-yl)-4- fluoroisoquinolin-1(2H)- one | 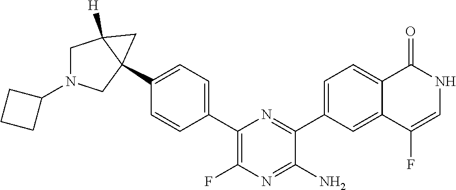 [SMILES: Nc1nc(F)c(-c2ccc([C@]34C[C@H]3CN(C3CCC3)C4)cc2)nc1-c1ccc2c(=O)[nH]cc(F)c2c1] | A | |
| I-100 | 6-(3-amino-6-(3- ((dimethylamino)methyl)- 4-(tetrahydro-2H- pyran-4-yl)phenyl)-5- fluoropyrazin-2-yl)-8- fluoro-3- methylisoquinolin- 1(2H)-one | 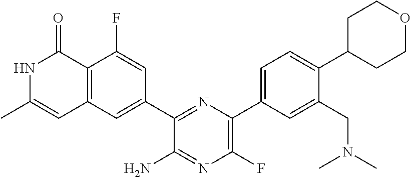 [SMILES: Cc1cc2cc(-c3nc(-c4ccc(C5CCOCC5)c(CN(C)C)c4)c(F)nc3N)cc(F)c2c(=O)[nH]1] | A | D |
| I-101 | 6-(3-amino-6-(3- ((dimethylamino)methyl)- 4-morpholinophenyl)- 5-fluoropyrazin-2-yl)-4- fluoro-3- methylisoquinolin- 1(2H)-one | 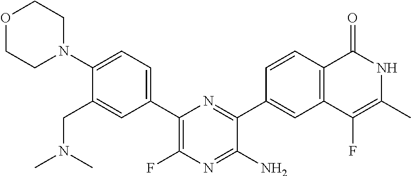 [SMILES: Cc1[nH]c(=O)c2ccc(-c3nc(-c4ccc(N5CCOCC5)c(CN(C)C)c4)c(F)nc3N)cc2c1F] | A | |
| I-102 | 6-(3-amino-5-fluoro-6- (4-(4- isopropylpiperazin-1- yl)phenyl)pyrazin-2-yl)- 4-chloro-8-fluoro-3- methylisoquinolin- 1(2H)-one | 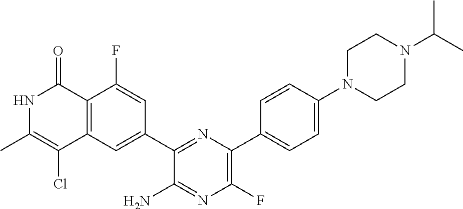 [SMILES: Cc1[nH]c(=O)c2c(F)cc(-c3nc(-c4ccc(N5CCN(C(C)C)CC5)cc4)c(F)nc3N)cc2c1Cl] | A | |
| I-103 | 6′-(3-amino-5-fluoro-6- (4-(4- isopropylpiperazin-1- yl)phenyl)pyrazin-2-yl)- 2′, 3′-dihydro-1′H- spiro[cyclopropane- 1,4′-isoquinolin]-1′-one | 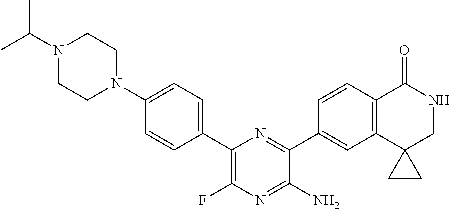 [SMILES: CC(C)N1CCN(c2ccc(-c3nc(-c4ccc5c(c4)C4(CC4)CNC5=O)c(N)nc3F)cc2)CC1] | A | C |
| I-104 | 6-(3-amino-6-(3- ((dimethylamino)methyl)- 4-(tetrahydro-2H- pyran-4-yl)phenyl)-5- fluoropyrazin-2-yl)-4- chloro-8-fluoro-3- methylisoquinolin- 1(2H)-one | 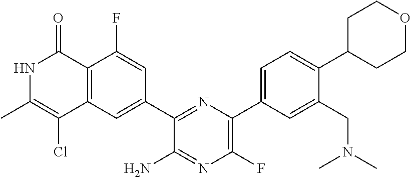 [SMILES: Cc1[nH]c(=O)c2c(F)cc(-c3nc(-c4ccc(C5CCOCC5)c(CN(C)C)c4)c(F)nc3N)cc2c1Cl] | A | |
| I-105 | 6′-(3-amino-6-(3- ((dimethylamino)methyl)- 4-morpholinophenyl)- 5-fluoropyrazin-2-yl)- 2′, 3′-dihydro-1′H- spiro[cyclopropane- 1,4′-isoquinolin]-1′-one | 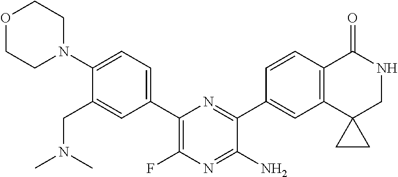 [SMILES: CN(C)Cc1cc(-c2nc(-c3ccc4c(c3)C3(CC3)CNC4=O)c(N)nc2F)ccc1N1CCOCC1] | A | |
| I-106 | 6-(3-amino-6-(4-(4- (cyclopropylmethyl) piperazin-1-yl) phenyl)-5- fluoropyrazin-2-yl)-4- fluoro-3- methylisoquinolin- 1(2H)-one | 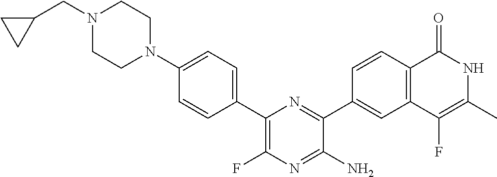 [SMILES: Cc1[nH]c(=O)c2ccc(-c3nc(-c4ccc(N5CCN(CC6CC6)CC5)cc4)c(F)nc3N)cc2c1F] | A | |
| I-107 | 6-(3-amino-6-(4-(4- (cyclopropylmethyl) piperazin-1-yl)phenyl)-5- fluoropyrazin-2-yl)-4- chloro-8-fluoro-3- methylisoquinolin- 1(2H)-one | 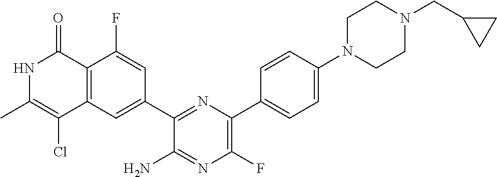 [SMILES: Cc1[nH]c(=O)c2c(F)cc(-c3nc(-c4ccc(N5CCN(CC6CC6)CC5)cc4)c(F)nc3N)cc2c1Cl] | A | |
| I-108 | 6′-(3-amino-6-(4-(4- (cyclopropylmethyl) piperazin-1-yl)phenyl)-5- fluoropyrazin-2-yl)-2′,3′- dihydro-1′H- spiro[cyclopropane- 1,4′-isoquinolin]-1′-one | 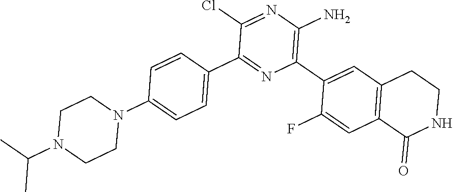 [SMILES: CC(C)N1CCN(c2ccc(-c3nc(-c4cc5c(cc4F)C(=O)NCC5)c(N)nc3Cl)cc2)CC1] | A | |
| I-109 | 6-(3-amino-6-(3- ((dimethylamino)methyl)- 4-morpholinophenyl)- 5-fluoropyrazin-2-yl)-4- chloro-8-fluoro-3- methylisoquinolin- 1(2H)-one | 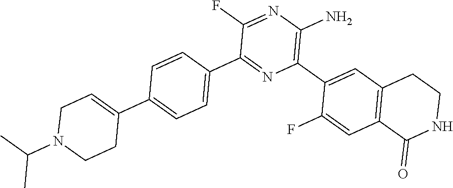 [SMILES: CC(C)N1CC=C(c2ccc(-c3nc(-c4cc5c(cc4F)C(=O)NCC5)c(N)nc3F)cc2)CC1] | A | |
| I-110 | 6′-(3-amino-6-(3- ((dimethylamino)methyl)- 4-(tetrahydro-2H- pyran-4-yl)phenyl)-5- fluoropyrazin-2-yl)-2′,3′- dihydro-1′H- spiro[cyclopropane- 1,4′-isoquinolin]-1′-one | 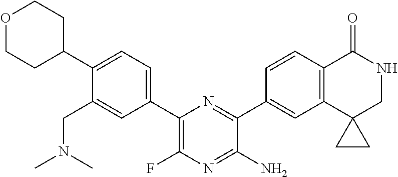 [SMILES: CN(C)Cc1cc(-c2nc(-c3ccc4c(c3)C3(CC3)CNC4=O)c(N)nc2F)ccc1C1CCOCC1] | A | C |
| I-111 | 6-(3-amino-5-fluoro-6- (4-(4- isopropylpiperazin-1- yl)phenyl)pyrazin-2-yl)- 4,8-difluoro-3- methylisoquinolin- 1(2H)-one | 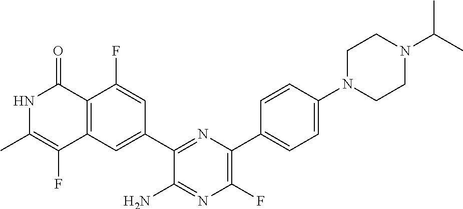 [SMILES: Cc1[nH]c(=O)c2c(F)cc(-c3nc(-c4ccc(N5CCN(C(C)C)CC5)cc4)c(F)nc3N)cc2c1F] | A | |
| I-112 | 6-(3-amino-5-fluoro-6- (4-morpholino-3- (pyrrolidin-1- ylmethyl)phenyl)pyrazin- 2-yl)-8-fluoro-3,4- dihydroisoquinolin- 1(2H)-one | 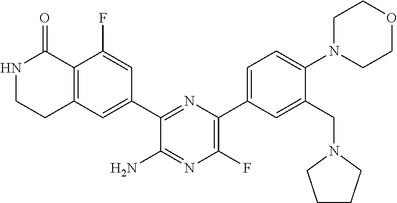 [SMILES: Nc1nc(F)c(-c2ccc(N3CCOCC3)c(CN3CCCC3)c2)nc1-c1cc(F)c2c(c1)CCNC2=O] | A | |
| I-113 | 6-(3-amino-5-fluoro-6- (4-morpholino-3- (pyrrolidin-1- ylmethyl)phenyl)pyrazin- 2-yl)-3,4- dihydroisoquinolin- 1(2H)-one | 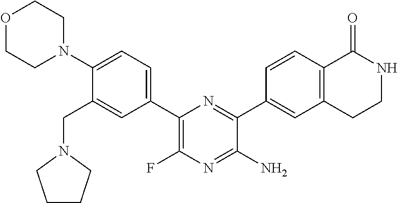 [SMILES: Nc1nc(F)c(-c2ccc(N3CCOCC3)c(CN3CCCC3)c2)nc1-c1ccc2c(c1)CCNC2=O] | A | D |
| I-114 | 6-(3-amino-5-fluoro-6- (4-morpholino-3- (pyrrolidin-1- ylmethyl)phenyl)pyrazin- 2-yl)isoquinolin-1(2H)- one | 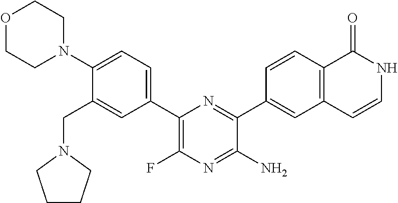 [SMILES: Nc1nc(F)c(-c2ccc(N3CCOCC3)c(CN3CCCC3)c2)nc1-c1ccc2c(=O)[nH]ccc2c1] | A | |
| I-115 | 6-(3-amino-6-(4-(4- (cyclopropylmethyl) piperazin-1-yl)phenyl)-5- fluoropyrazin-2-yl)-4,8- difluoro-3- methylisoquinolin- 1(2H)-one | 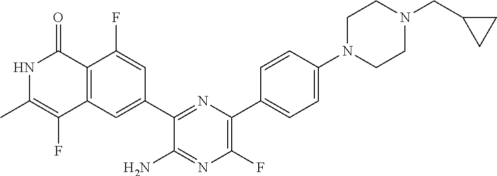 [SMILES: Cc1[nH]c(=O)c2c(F)cc(-c3nc(-c4ccc(N5CCN(CC6CC6)CC5)cc4)c(F)nc3N)cc2c1F] | A | |
| I-116 | 6-(3-amino-6-(3- ((dimethylamino)methyl)- 4-morpholinophenyl)- 5-fluoropyrazin-2-yl)- 4,8-difluoro-3- methylisoquinolin- 1(2H)-one | 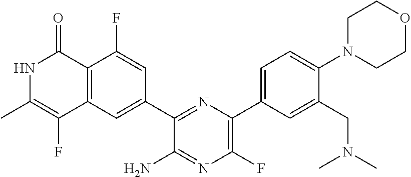 [SMILES: Cc1[nH]c(=O)c2c(F)cc(-c3nc(-c4ccc(N5CCOCC5)c(CN(C)C)c4)c(F)nc3N)cc2c1F] | A | |
| I-117 | 6-(3-amino-6-(3- ((dimethylamino)methyl)- 4-(tetrahydro-2H- pyran-4-yl)phenyl)-5- fluoropyrazin-2-yl)-4,8- difluoro-3- methylisoquinolin- 1(2H)-one | 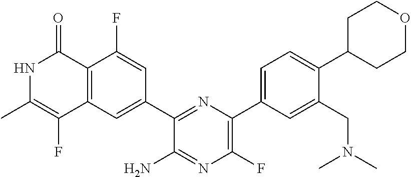 [SMILES: Cc1[nH]c(=O)c2c(F)cc(-c3nc(-c4ccc(C5CCOCC5)c(CN(C)C)c4)c(F)nc3N)cc2c1F] | A | D |
| I-118 | 6-(3-amino-5-fluoro-6- (3-((4- methoxypiperidin-1- yl)methyl)-4- morpholinophenyl) pyrazin-2-yl)- 7-fluoro-3,4- dihydroisoquinolin- 1(2H)-one | 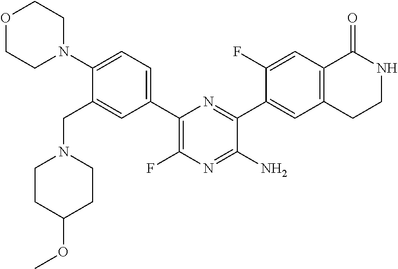 [SMILES: COC1CCN(Cc2cc(-c3nc(-c4cc5c(cc4F)C(=O)NCC5)c(N)nc3F)ccc2N2CCOCC2)CC1] | B | |
| I-119 | 6-(3-amino-5-fluoro-6- (4-(4- isopropylpiperazin-1- yl)phenyl)pyrazin-2-yl)- 4-methoxyisoquinolin- 1(2H)-one | 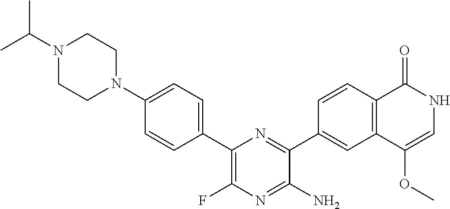 [SMILES: COc1c[nH]c(=O)c2ccc(-c3nc(-c4ccc(N5CCN(C(C)C)CC5)cc4)c(F)nc3N)cc12] | B | |
| I-120 | 6-(3-amino-6-(3- ((dimethylamino)methyl)- 4-morpholinophenyl)- 5-fluoropyrazin-2-yl)- 4,8-difluoroisoquinolin- 1(2H)-one | 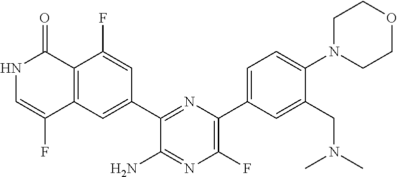 [SMILES: CN(C)Cc1cc(-c2nc(-c3cc(F)c4c(=O)[nH]cc(F)c4c3)c(N)nc2F)ccc1N1CCOCC1] | A | |
| I-121 | 6-(3-amino-6-(3- ((dimethylamino)methyl)- 4-(tetrahydro-2H- pyran-4-yl)phenyl)-5- fluoropyrazin-2-yl)-4,8- difluoroisoquinolin- 1(2H)-one | 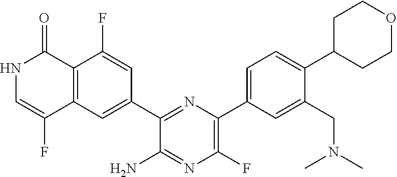 [SMILES: CN(C)Cc1cc(-c2nc(-c3cc(F)c4c(=O)[nH]cc(F)c4c3)c(N)nc2F)ccc1C1CCOCC1] | A | D |
| I-122 | 6-(3-amino-6-(4-(4- (cyclopropylmethyl) piperazin-1-yl)phenyl)-5- fluoropyrazin-2-yl)-4- chloro-3- methylisoquinolin- 1(2H)-one | 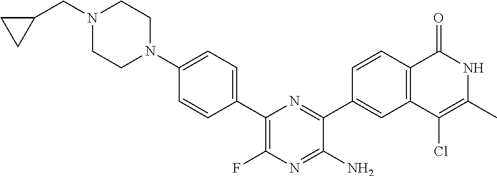 [SMILES: Cc1[nH]c(=O)c2ccc(-c3nc(-c4ccc(N5CCN(CC6CC6)CC5)cc4)c(F)nc3N)cc2c1Cl] | A | |
| I-123 | 6-(3-amino-5-fluoro-6- (4-(8-methyl-5-oxa-2,8- diazaspiro[3.5]nonan-2- yl)phenyl)pyrazin-2-yl)- 4-fluoro-3- methylisoquinolin- 1(2H)-one | 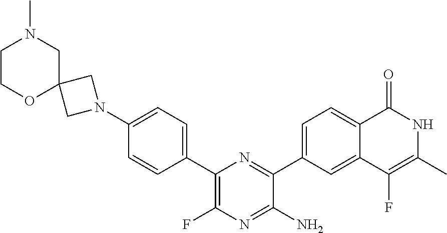 [SMILES: Cc1[nH]c(=O)c2ccc(-c3nc(-c4ccc(N5CC6(CN(C)CCO6)C5)cc4)c(F)nc3N)cc2c1F] | B | |
| I-124 | 6-(3-amino-6-(3- ((dimethylamino)methyl)- 4-morpholinophenyl)- 5-fluoropyrazin-2-yl)-4- chloro-3- methylisoquinolin- 1(2H)-one | 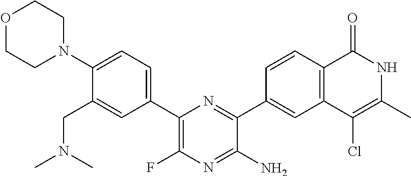 [SMILES: Cc1[nH]c(=O)c2ccc(-c3nc(-c4ccc(N5CCOCC5)c(CN(C)C)c4)c(F)nc3N)cc2c1Cl] | A | |
| I-125 | 6-(3-amino-6-(3- ((dimethylamino)methyl)- 4-(tetrahydro-2H- pyran-4-yl)phenyl)-5- fluoropyrazin-2-yl)-4- chloro-3- methylisoquinolin- 1(2H)-one | 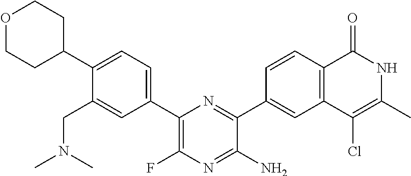 [SMILES: Cc1[nH]c(=O)c2ccc(-c3nc(-c4ccc(C5CCOCC5)c(CN(C)C)c4)c(F)nc3N)cc2c1Cl] | A | |
| I-126 | 6-(3-amino-5-fluoro-6- (3-((4- methoxypiperidin-1- yl)methyl)-4- thiomorpholinophenyl) pyrazin-2-yl)-3,4- dihydroisoquinolin- 1(2H)-one | 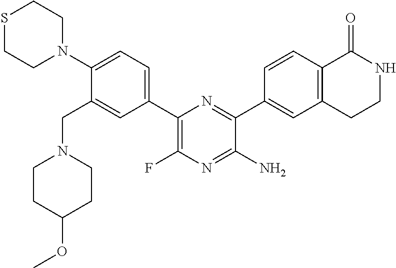 [SMILES: COC1CCN(Cc2cc(-c3nc(-c4ccc5c(c4)CCNC5=O)c(N)nc3F)ccc2N2CCSCC2)CC1] | B | |
| I-127 | 6-(3-amino-5-fluoro-6- (3-((4- methoxypiperidin-1- yl)methyl)-4- thiomorpholinophenyl) pyrazin-2-yl)-7-fluoro- 3,4-dihydroisoquinolin- 1(2H)-one | 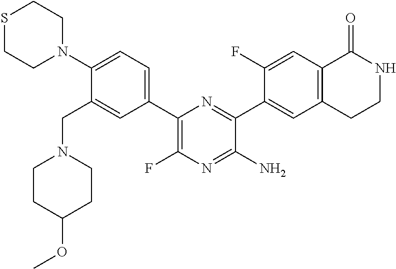 [SMILES: COC1CCN(Cc2cc(-c3nc(-c4cc5c(cc4F)C(=O)NCC5)c(N)nc3F)ccc2N2CCSCC2)CC1] | B | |
| I-128 | 6-(3-amino-6-(3- ((dimethylamino)methyl)- 4-thiomorpholinophenyl)- 5-fluoropyrazin-2-yl)- 3,4-dihydroisoquinolin- 1(2H)-one | 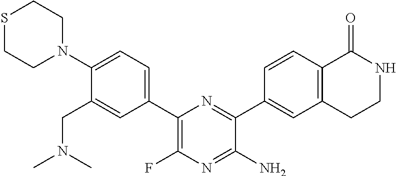 [SMILES: CN(C)Cc1cc(-c2nc(-c3ccc4c(c3)CCNC4=O)c(N)nc2F)ccc1N1CCSCC1] | A | |
| I-129 | 6-(3-amino-6-(3- ((dimethylamino) methyl)-4- thiomorpholinophenyl)- 5-fluoropyrazin-2-yl)-7- fluoro-3,4- dihydroisoquinolin- 1(2H)-one | 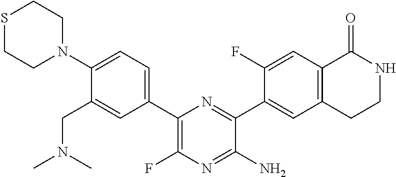 [SMILES: CN(C)Cc1cc(-c2nc(-c3cc4c(cc3F)C(=O)NCC4)c(N)nc2F)ccc1N1CCSCC1] | A | |
| I-130 | 7-(3-amino-5-fluoro-6- (4-(8-methyl-5-oxa-2,8- diazaspiro[3.5]nonan-2- yl)phenyl)pyrazin-2-yl)- 2-methylquinazolin- 4(3H)-one | 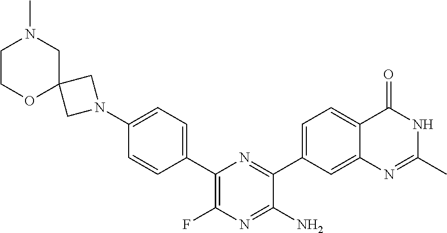 [SMILES: Cc1nc2cc(-c3nc(-c4ccc(N5CC6(CN(C)CCO6)C5)cc4)c(F)nc3N)ccc2c(=O)[nH]1] | C | |
| I-131 | 6-(3-amino-5-fluoro-6- (3-((4- methoxypiperidin-1- yl)methyl)-4- (tetrahydro-2H-pyran-4- yl)phenyl)pyrazin-2-yl)- 3,4-dihydroisoquinolin- 1(2H)-one | 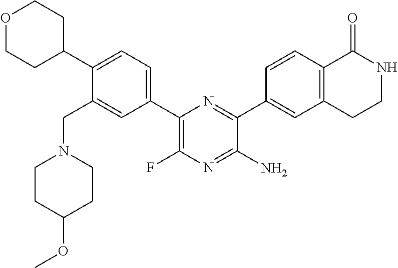 [SMILES: COC1CCN(Cc2cc(-c3nc(-c4ccc5c(c4)CCNC5=O)c(N)nc3F)ccc2C2CCOCC2)CC1] | B | |
| I-132 | 6-(3-amino-5-fluoro-6- (3-((4- methoxypiperidin-1- yl)methyl)-4- (tetrahydro-2H-pyran-4- yl)phenyl)pyrazin-2-yl)- 7-fluoro-3,4- dihydroisoquinolin- 1(2H)-one | 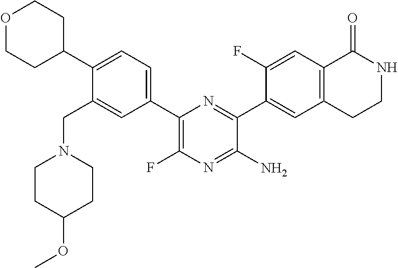 [SMILES: COC1CCN(Cc2cc(-c3nc(-c4cc5c(cc4F)C(=O)NCC5)c(N)nc3F)ccc2C2CCOCC2)CC1] | B | |
| I-133 | 6-(3-amino-5-fluoro-6- (3-((3- methoxypyrrolidin-1- yl)methyl)-4- morpholinophenyl) pyrazin-2-yl)-3,4- dihydroisoquinolin- 1(2H)-one | 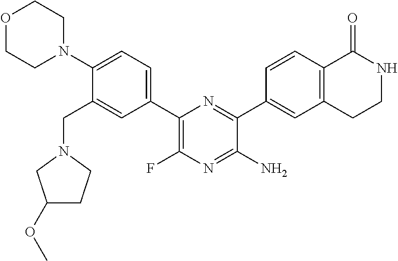 [SMILES: COC1CCN(Cc2cc(-c3nc(-c4ccc5c(c4)CCNC5=O)c(N)nc3F)ccc2N2CCOCC2)C1] | A | D |
| I-134 | 6-(3-amino-5-fluoro-6- (3-((3- methoxypyrrolidin-1- yl)methyl)-4- morpholinophenyl) pyrazin-2-yl)-7-fluoro- 3,4-dihydroisoquinolin- 1(2H)-one | 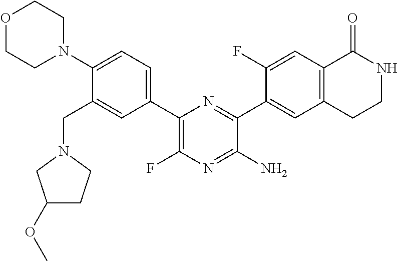 [SMILES: COC1CCN(Cc2cc(-c3nc(-c4cc5c(cc4F)C(=O)NCC5)c(N)nc3F)ccc2N2CCOCC2)C1] | A | |
| I-135 | 6-(3-amino-5-fluoro-6- (4-((1S,5R)-3-methyl-3- azabicyclo[3.1.0]hexan- 1-yl)phenyl)pyrazin-2- yl)-4,4,8-trifluoro-3- methyl-3,4- dihydroisoquinolin- 1(2H)-one | 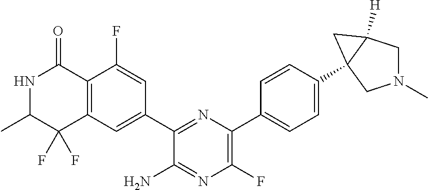 | B | |
| I-136 | 6-(3-amino-6-(4-(4-(4,4- difluorobutyl)piperazin- 1-yl)phenyl)-5- fluoropyrazin-2-yl)-4- fluoroisoquinolin-1(2H)- one | 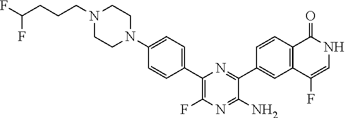 [SMILES: Nc1nc(F)c(-c2ccc(N3CCN(CCCC(F)F)CC3)cc2)nc1-c1ccc2c(=O)[nH]cc(F)c2c1] | A | |
| I-137 | 6-(3-amino-6-(4-(4-(4,4- difluorobutyl)piperazin- 1-yl)phenyl)-5- fluoropyrazin-2-yl)-8- fluoro-3,4- dihydroisoquinolin- 1(2H)-one | 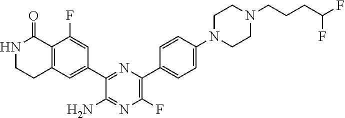 [SMILES: Nc1nc(F)c(-c2ccc(N3CCN(CCCC(F)F)CC3)cc2)nc1-c1cc(F)c2c(c1)CCNC2=O] | A | |
| I-138 | 6-(3-amino-5-fluoro-6- (3-((4- methoxypiperidin-1- yl)methyl)-4- morpholinophenyl) pyrazin-2-yl)-3,4- dihydroisoquinolin- 1(2H)-one | 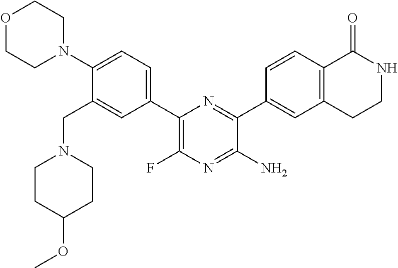 [SMILES: COC1CCN(Cc2cc(-c3nc(-c4ccc5c(c4)CCNC5=O)c(N)nc3F)ccc2N2CCOCC2)CC1] | B | |
| I-139 | 6-(3-amino-6-(4-(4- cyclobutylpiperazin-1- yl)phenyl)-5- fluoropyrazin-2-yl)-4- methylisoquinolin- 1(2H)-one | 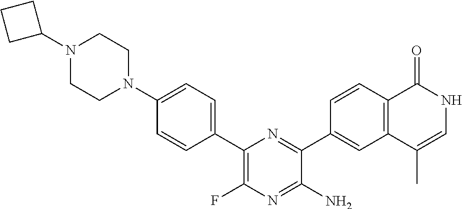 [SMILES: Cc1c[nH]c(=O)c2ccc(-c3nc(-c4ccc(N5CCN(C6CCC6)CC5)cc4)c(F)nc3N)cc12] | A | |
| I-140 | 6-(3-amino-5-fluoro-6- (4-(4- isopropylpiperazin-1- yl)phenyl)pyrazin-2-yl)- 4,7-difluoroisoquinolin- 1(2H)-one | 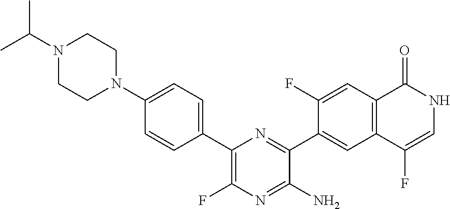 [SMILES: CC(C)N1CCN(c2ccc(-c3nc(-c4cc5c(F)c[nH]c(=O)c5cc4F)c(N)nc3F)cc2)CC1] | A | |
| I-141 | 6-(3-amino-6-(4-(4- (cyclopropylmethyl) piperazin-1-yl)phenyl)-5- fluoropyrazin-2-yl)-4,7- difluoroisoquinolin- 1(2H)-one | 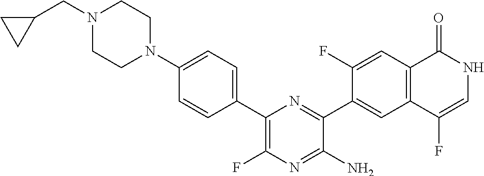 [SMILES: Nc1nc(F)c(-c2ccc(N3CCN(CC4CC4)CC3)cc2)nc1-c1cc2c(F)c[nH]c(=O)c2cc1F] | A | |
| I-142 | 6-(3-amino-6-(3- ((dimethylamino)methyl)- 4-morpholinophenyl)- 5-fluoropyrazin-2-yl)- 4,7-difluoroisoquinolin- 1(2H)-one | 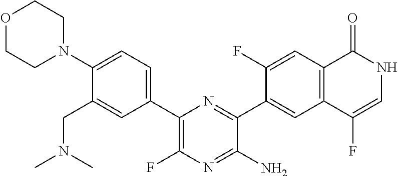 [SMILES: CN(C)Cc1cc(-c2nc(-c3cc4c(F)c[nH]c(=O)c4cc3F)c(N)nc2F)ccc1N1CCOCC1] | A | D |
| I-143 | 6-(3-amino-6-(3- ((dimethylamino)methyl)- 4-(tetrahydro-2H- pyran-4-yl)phenyl)-5- fluoropyrazin-2-yl)-4,7- difluoroisoquinolin- 1(2H)-one | 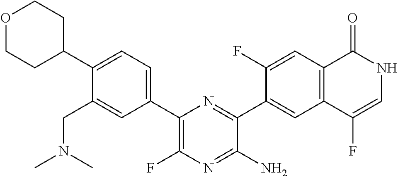 [SMILES: CN(C)Cc1cc(-c2nc(-c3cc4c(F)c[nH]c(=O)c4cc3F)c(N)nc2F)ccc1C1CCOCC1] | A | |
| I-144 | 6-(3-amino-6-(3- (azetidin-1-ylmethyl)-4- morpholinophenyl)-5- fluoropyrazin-2-yl)-3,4- dihydroisoquinolin- 1(2H)-one | 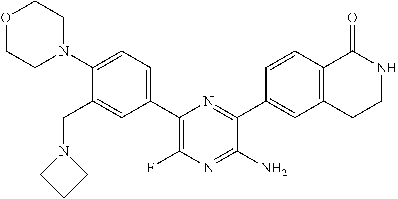 [SMILES: Nc1nc(F)c(-c2ccc(N3CCOCC3)c(CN3CCC3)c2)nc1-c1ccc2c(c1)CCNC2=O] | A | |
| I-145 | 6-(3-amino-6-(3- (azetidin-1-ylmethyl)-4- morpholinophenyl)-5- fluoropyrazin-2-yl)-7- fluoro-3,4- dihydroisoquinolin- 1(2H)-one | 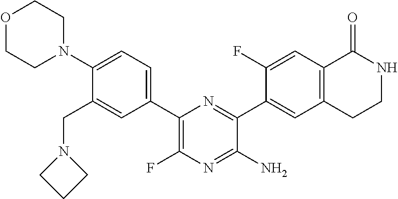 [SMILES: Nc1nc(F)c(-c2ccc(N3CCOCC3)c(CN3CCC3)c2)nc1-c1cc2c(cc1F)C(=O)NCC2] | A | D |
| I-146 | 6-(3-amino-6-(3- (azetidin-1-ylmethyl)-4- morpholinophenyl)-5- fluoropyrazin-2-yl)-8- fluoro-3,4- dihydroisoquinolin- 1(2H)-one | 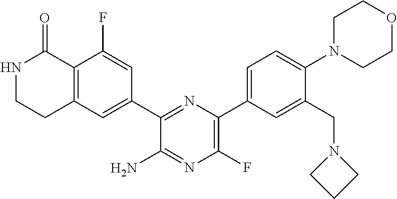 [SMILES: Nc1nc(F)c(-c2ccc(N3CCOCC3)c(CN3CCC3)c2)nc1-c1cc(F)c2c(c1)CCNC2=O] | A | D |
| I-147 | 6-(3-amino-5-fluoro-6- (3-(pyrrolidin-1- ylmethyl)-4-(tetrahydro- 2H-pyran-4- yl)phenyl)pyrazin-2-yl)- 3,4-dihydroisoquinolin- 1(2H)-one | 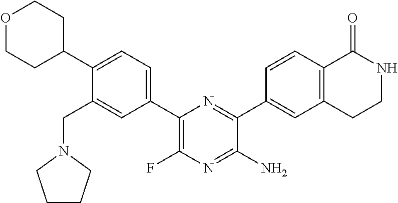 [SMILES: Nc1nc(F)c(-c2ccc(C3CCOCC3)c(CN3CCCC3)c2)nc1-c1ccc2c(c1)CCNC2=O] | A | |
| I-148 | 6-(3-amino-5-fluoro-6- (3-(pyrrolidin-1- ylmethyl)-4-(tetrahydro- 2H-pyran-4- yl)phenyl)pyrazin-2-yl)- 7-fluoro-3,4- dihydroisoquinolin- 1(2H)-one | 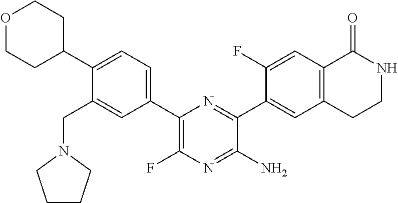 [SMILES: Nc1nc(F)c(-c2ccc(C3CCOCC3)c(CN3CCCC3)c2)nc1-c1cc2c(cc1F)C(=O)NCC2] | A | D |
| I-149 | 6-(3-amino-5-fluoro-6- (3-(pyrrolidin-1- ylmethyl)-4-(tetrahydro- 2H-pyran-4- yl)phenyl)pyrazin-2-yl)- 8-fluoro-3,4- dihydroisoquinolin- 1(2H)-one | 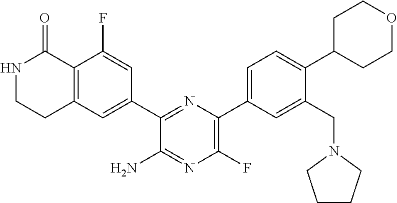 [SMILES: Nc1nc(F)c(-c2ccc(C3CCOCC3)c(CN3CCCC3)c2)nc1-c1cc(F)c2c(c1)CCNC2=O] | A | |
| I-150 | 6-(3-amino-6-(4-(4- cyclobutylpiperazin-1- yl)phenyl)-5- fluoropyrazin-2-yl)- 4,4,8-trifluoro-3-methyl- 3,4-dihydroisoquinolin- 1(2H)-one | 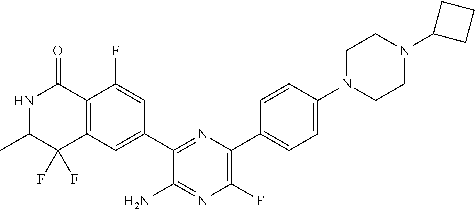 [SMILES: CC1NC(=O)c2c(F)cc(-c3nc(-c4ccc(N5CCN(C6CCC6)CC5)cc4)c(F)nc3N)cc2C1(F)F] | B | |
| I-151 | (R)-6-(3-amino-5- fluoro-6-(3-((3- methoxypyrrolidin-1- yl)methyl)-4- morpholinophenyl) pyrazin-2-yl)-3,4- dihydroisoquinolin- 1(2H)-one | 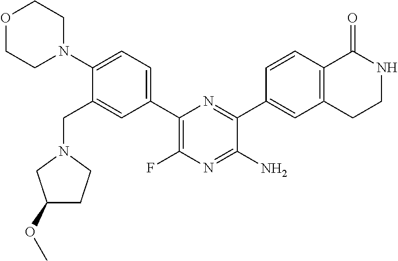 [SMILES: CO[C@@H]1CCN(Cc2cc(-c3nc(-c4ccc5c(c4)CCNC5=O)c(N)nc3F)ccc2N2CCOCC2)C1] | A | |
| I-152 | (R)-6-(3-amino-5- fluoro-6-(3-((3- methoxypyrrolidin-1- yl)methyl)-4- morpholinophenyl) pyrazin-2-yl)- 7-fluoro-3,4- dihydroisoquinolin- 1(2H)-one | 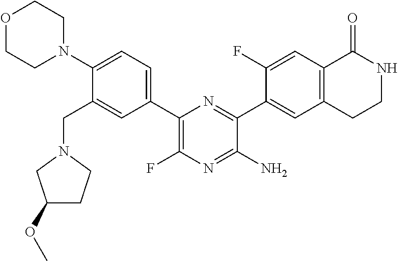 [SMILES: CO[C@@H]1CCN(Cc2cc(-c3nc(-c4cc5c(cc4F)C(=O)NCC5)c(N)nc3F)ccc2N2CCOCC2)C1] | A | |
| I-153 | 6-(3-amino-6-(3- ((dimethylamino)methyl)- 4-morpholinophenyl)- 5-fluoropyrazin-2-yl)-4- chloroisoquinolin- 1(2H)-one | 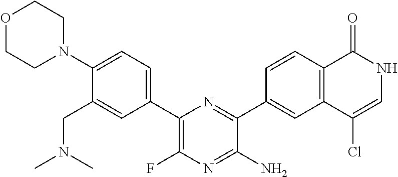 [SMILES: CN(C)Cc1cc(-c2nc(-c3ccc4c(=O)[nH]cc(Cl)c4c3)c(N)nc2F)ccc1N1CCOCC1] | A | |
| I-154 | 6-(3-amino-6-(3- ((dimethylamino)methyl)- 4-(tetrahydro-2H- pyran-4-yl)phenyl)-5- fluoropyrazin-2-yl)-4- chloroisoquinolin- 1(2H)-one | 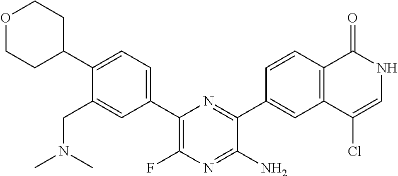 [SMILES: CN(C)Cc1cc(-c2nc(-c3ccc4c(=O)[nH]cc(Cl)c4c3)c(N)nc2F)ccc1C1CCOCC1] | A | D |
| I-155 | (R)-6-(3-amino-5- fluoro-6-(3-((3- methoxypyrrolidin-1- yl)methyl)-4- morpholinophenyl) pyrazin-2-yl)-8-fluoro- 3,4-dihydroisoquinolin- 1(2H)-one | 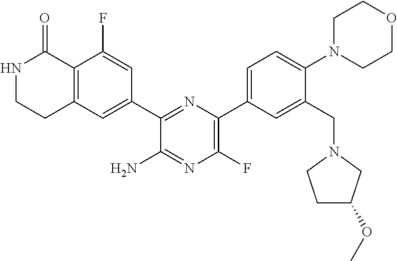 [SMILES: CO[C@@H]1CCN(Cc2cc(-c3nc(-c4cc(F)c5c(c4)CCNC5=O)c(N)nc3F)ccc2N2CCOCC2)C1] | A | |
| I-156 | (S)-6-(3-amino-5-fluoro- 6-(3-((3- methoxypyrrolidin-1- yl)methyl)-4- morpholinophenyl) pyrazin-2-yl)-3,4- dihydroisoquinolin- 1(2H)-one | 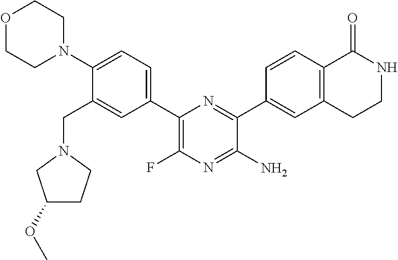 [SMILES: CO[C@H]1CCN(Cc2cc(-c3nc(-c4ccc5c(c4)CCNC5=O)c(N)nc3F)ccc2N2CCOCC2)C1] | A | |
| I-157 | (S)-6-(3-amino-5-fluoro- 6-(3-((3- methoxypyrrolidin-1- yl)methyl)-4- morpholinophenyl) pyrazin-2-yl)- 7-fluoro-3,4- dihydroisoquinolin- 1(2H)-one | 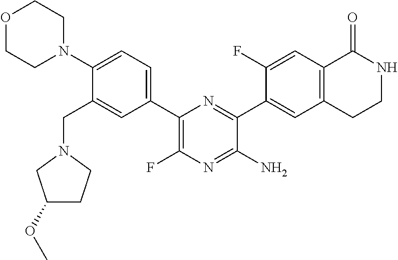 [SMILES: CO[C@H]1CCN(Cc2cc(-c3nc(-c4cc5c(cc4F)C(=O)NCC5)c(N)nc3F)ccc2N2CCOCC2)C1] | A | |
| I-158 | (S)-6-(3-amino-5-fluoro- 6-(3-((3- methoxypyrrolidin-1- yl)methyl)-4- morpholinophenyl) pyrazin-2-yl)- 8-fluoro-3,4- dihydroisoquinolin- 1(2H)-one | 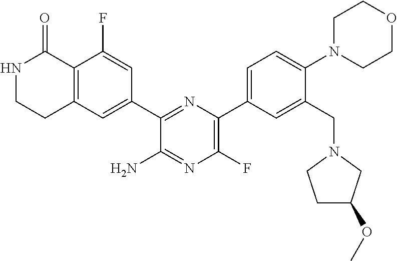 [SMILES: CO[C@H]1CCN(Cc2cc(-c3nc(-c4cc(F)c5c(c4)CCNC5=O)c(N)nc3F)ccc2N2CCOCC2)C1] | A | |
| I-159 | 6-(3-amino-6-(4-(4-(4,4- difluorobutyl)piperazin- 1-yl)phenyl)-5- fluoropyrazin-2-yl)-8- fluoro-3- methylisoquinolin- 1(2H)-one | 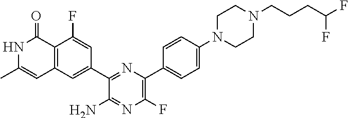 [SMILES: Cc1cc2cc(-c3nc(-c4ccc(N5CCN(CCCC(F)F)CC5)cc4)c(F)nc3N)cc(F)c2c(=O)[nH]1] | A | |
| I-160 | 6-(3-amino-5-fluoro-6- (3-(((2- methoxyethyl)(methyl) amino)methyl)-4- morpholinophenyl) pyrazin-2-yl)-3,4- dihydroisoquinolin- 1(2H)-one | 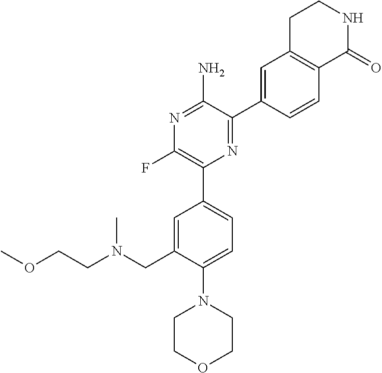 [SMILES: COCCN(C)Cc1cc(-c2nc(-c3ccc4c(c3)CCNC4=O)c(N)nc2F)ccc1N1CCOCC1] | A | |
| I-161 | 6-(3-amino-5-fluoro-6- (3-(((2- methoxyethyl)(methyl) amino)methyl)-4- morpholinophenyl) pyrazin-2-yl)- 7-fluoro-3,4- dihydroisoquinolin- 1(2H)-one | 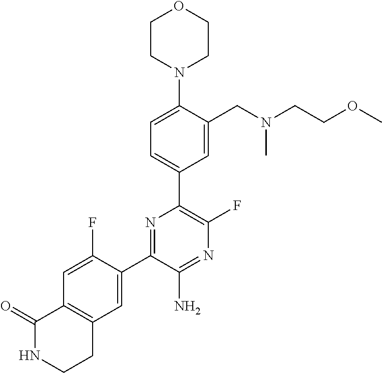 [SMILES: COCCN(C)Cc1cc(-c2nc(-c3cc4c(cc3F)C(=O)NCC4)c(N)nc2F)ccc1N1CCOCC1] | A | |
| I-162 | 6-(3-amino-5-fluoro-6- (3-(((2- methoxyethyl)(methyl) amino)methyl)-4- morpholinophenyl) pyrazin-2-yl)-8-fluoro- 3,4-dihydroisoquinolin- 1(2H)-one | 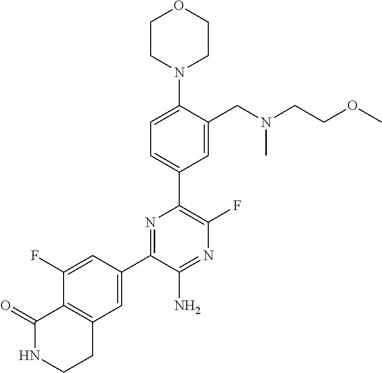 [SMILES: COCCN(C)Cc1cc(-c2nc(-c3cc(F)c4c(c3)CCNC4=O)c(N)nc2F)ccc1N1CCOCC1] | A | |
| I-163 | 6-(3-amino-6-(3- ((ethyl(methyl)amino) methyl)-4-(tetrahydro-2H- pyran-4-yl)phenyl)-5- fluoropyrazin-2-yl)-3,4- dihydroisoquinolin- 1(2H)-one | 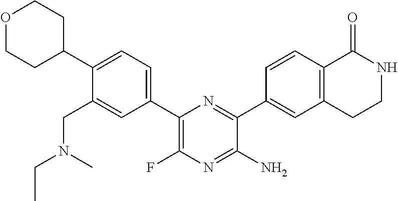 [SMILES: CCN(C)Cc1cc(-c2nc(-c3ccc4c(c3)CCNC4=O)c(N)nc2F)ccc1C1CCOCC1] | A | |
| I-164 | 6-(3-amino-6-(3- ((ethyl(methyl)amino) methyl)-4-(tetrahydro-2H- pyran-4-yl)phenyl)-5- fluoropyrazin-2-yl)-7- fluoro-3,4- dihydroisoquinolin- 1(2H)-one | 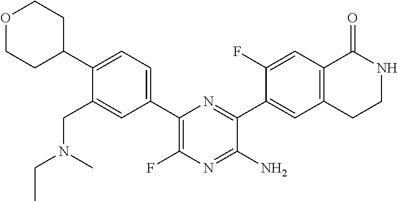 [SMILES: CCN(C)Cc1cc(-c2nc(-c3cc4c(cc3F)C(=O)NCC4)c(N)nc2F)ccc1C1CCOCC1] | A | D |
| I-165 | 6-(3-amino-6-(3- ((ethyl(methyl)amino) methyl)-4-(tetrahydro-2H- pyran-4-yl)phenyl)-5- fluoropyrazin-2-yl)-8- fluoro-3,4- dihydroisoquinolin- 1(2H)-one | 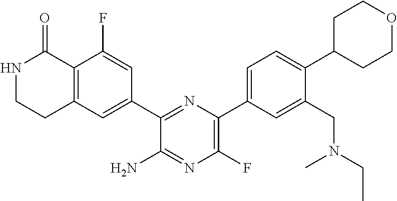 [SMILES: CCN(C)Cc1cc(-c2nc(-c3cc(F)c4c(c3)CCNC4=O)c(N)nc2F)ccc1C1CCOCC1] | A | D |
| I-166 | 6-(3-amino-5-fluoro-6- (4-(4- isopropylpiperazin-1- yl)phenyl)pyrazin-2-yl)- 8-fluoro-4- methylisoquinolin- 1(2H)-one | 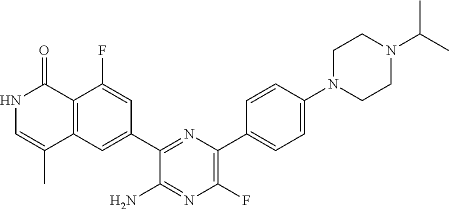 [SMILES: Cc1c[nH]c(=O)c2c(F)cc(-c3nc(-c4ccc(N5CCN(C(C)C)CC5)cc4)c(F)nc3N)cc12] | A | |
| I-167 | 6-(3-amino-6-(3- ((dimethylamino)methyl)- 4-(tetrahydro-2H- pyran-4-yl)phenyl)-5- fluoropyrazin-2-yl)-8- fluoro-4- methylisoquinolin- 1(2H)-one | 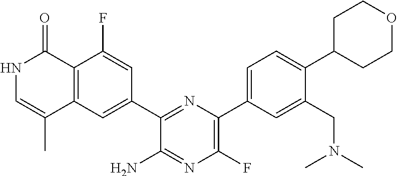 [SMILES: Cc1c[nH]c(=O)c2c(F)cc(-c3nc(-c4ccc(C5CCOCC5)c(CN(C)C)c4)c(F)nc3N)cc12] | A | |
| I-168 | 6-(3-amino-5-fluoro-6- (4-(4- isopropylpiperazin-1- yl)phenyl)pyrazin-2-yl)- 4,7-difluoro-3- methylisoquinolin- 1(2H)-one | 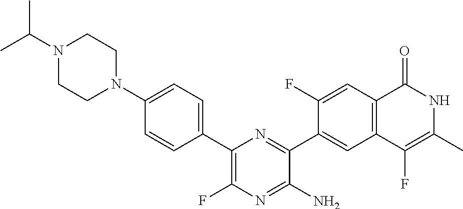 [SMILES: Cc1[nH]c(=O)c2cc(F)c(-c3nc(-c4ccc(N5CCN(C(C)C)CC5)cc4)c(F)nc3N)cc2c1F] | A | |
| I-169 | 6-(3-amino-6-(3- (azetidin-1-ylmethyl)-4- (tetrahydro-2H-pyran-4- yl)phenyl)-5- fluoropyrazin-2-yl)-3,4- dihydroisoquinolin- 1(2H)-one | 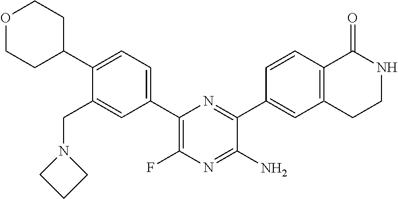 [SMILES: Nc1nc(F)c(-c2ccc(C3CCOCC3)c(CN3CCC3)c2)nc1-c1ccc2c(c1)CCNC2=O] | A | |
| I-170 | 6-(3-amino-6-(3- (azetidin-1-ylmethyl)-4- (tetrahydro-2H-pyran-4- yl)phenyl)-5- fluoropyrazin-2-yl)-7- fluoro-3,4- dihydroisoquinolin- 1(2H)-one | 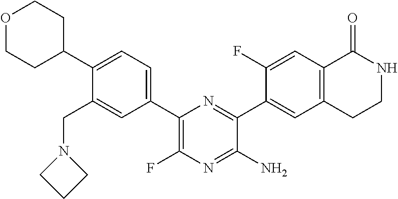 [SMILES: Nc1nc(F)c(-c2ccc(C3CCOCC3)c(CN3CCC3)c2)nc1-c1cc2c(cc1F)C(=O)NCC2] | A | D |
| I-171 | 6-(3-amino-6-(3- (azetidin-1-ylmethyl)-4- (tetrahydro-2H-pyran-4- yl)phenyl)-5- fluoropyrazin-2-yl)-8- fluoro-3,4- dihydroisoquinolin- 1(2H)-one | 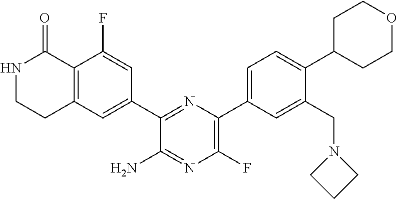 [SMILES: Nc1nc(F)c(-c2ccc(C3CCOCC3)c(CN3CCC3)c2)nc1-c1cc(F)c2c(c1)CCNC2=O] | A | C |
| I-172 | (R)-6-(3-amino-6-(3- ((dimethylamino)methyl)- 4-(2- methylmorpholino) phenyl)-5-fluoropyrazin- 2-yl)-3,4- dihydroisoquinolin- 1(2H)-one | 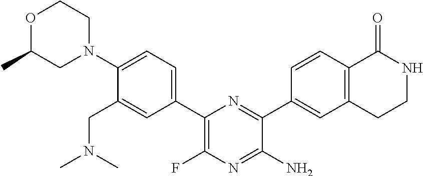 [SMILES: C[C@@H]1CN(c2ccc(-c3nc(-c4ccc5c(c4)CCNC5=O)c(N)nc3F)cc2CN(C)C)CCO1] | A | D |
| I-173 | (R)-6-(3-amino-6-(3- ((dimethylamino)methyl)- 4-(2-methylmorpholino) phenyl)-5- fluoropyrazin-2- yl)-7-fluoro-3,4- dihydroisoquinolin- 1(2H)-one | 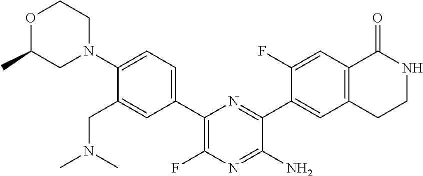 [SMILES: C[C@@H]1CN(c2ccc(-c3nc(-c4cc5c(cc4F)C(=O)NCC5)c(N)nc3F)cc2CN(C)C)CCO1] | A | C |
| I-174 | 6-(3-amino-5-fluoro-6- (3-(((2- methoxyethyl)(methyl) amino)methyl)-4- (tetrahydro-2H-pyran-4- yl)phenyl)pyrazin-2-yl)- 3,4-dihydroisoquinolin- 1(2H)-one | 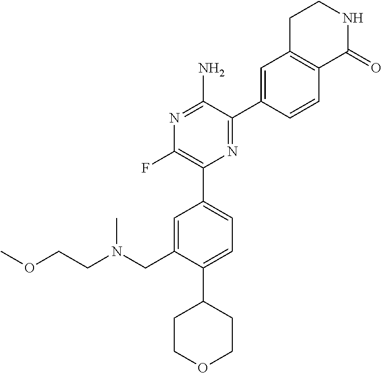 [SMILES: COCCN(C)Cc1cc(-c2nc(-c3ccc4c(c3)CCNC4=O)c(N)nc2F)ccc1C1CCOCC1] | A | |
| I-175 | 6-(3-amino-5-fluoro-6- (3-(((2- methoxyethyl)(methyl) amino)methyl)-4- (tetrahydro-2H-pyran-4- yl)phenyl)pyrazin-2-yl)- 7-fluoro-3,4- dihydroisoquinolin- 1(2H)-one | 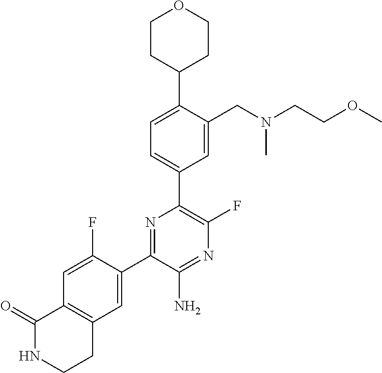 [SMILES: COCCN(C)Cc1cc(-c2nc(-c3cc4c(cc3F)C(=O)NCC4)c(N)nc2F)ccc1C1CCOCC1] | A | |
| I-176 | 6-(3-amino-5-fluoro-6- (3-(((2- methoxyethyl)(methyl) amino)methyl)-4- (tetrahydro-2H-pyran-4- yl)phenyl)pyrazin-2-yl)- 8-fluoro-3,4- dihydroisoquinolin- 1(2H)-one | 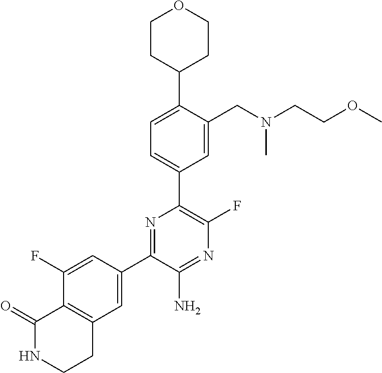 [SMILES: COCCN(C)Cc1cc(-c2nc(-c3cc(F)c4c(c3)CCNC4=O)c(N)nc2F)ccc1C1CCOCC1] | A | |
| I-177 | (R)-6-(3-amino-6-(3- ((dimethylamino)methyl)- 4-(2- methylmorpholino) phenyl)-5-fluoropyrazin- 2-yl)-8-fluoro-3,4- dihydroisoquinolin- 1(2H)-one | 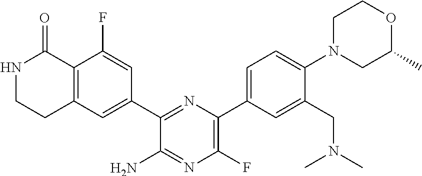 [SMILES: C[C@@H]1CN(c2ccc(-c3nc(-c4cc(F)c5c(c4)CCNC5=O)c(N)nc3F)cc2CN(C)C)CCO1] | A | |
| I-178 | 6-(3-amino-6-(3- ((dimethylamino)methyl)- 4-((tetrahydro-2H- pyran-4- yl)methoxy)phenyl)-5- fluoropyrazin-2-yl)-3,4- dihydroisoquinolin- 1(2H)-one | 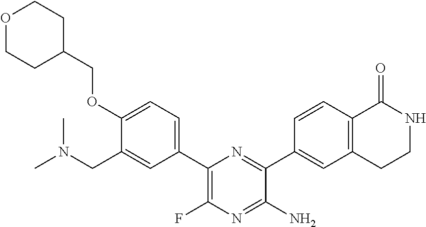 [SMILES: CN(C)Cc1cc(-c2nc(-c3ccc4c(c3)CCNC4=O)c(N)nc2F)ccc1OCC1CCOCC1] | A | |
| I-179 | 6-(3-amino-6-(3- ((dimethylamino)methyl)- 4-((tetrahydro-2H- pyran-4- yl)methoxy)phenyl)-5- fluoropyrazin-2-yl)-7- fluoro-3,4- dihydroisoquinolin- 1(2H)-one | 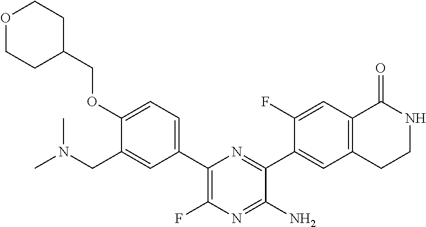 [SMILES: CN(C)Cc1cc(-c2nc(-c3cc4c(cc3F)C(=O)NCC4)c(N)nc2F)ccc1OCC1CCOCC1] | A | |
| I-180 | 6-(3-amino-6-(3- ((dimethylamino)methyl)- 4-((tetrahydro-2H- pyran-4- yl)methoxy)phenyl)-5- fluoropyrazin-2-yl)-8- fluoro-3,4- dihydroisoquinolin- 1(2H)-one | 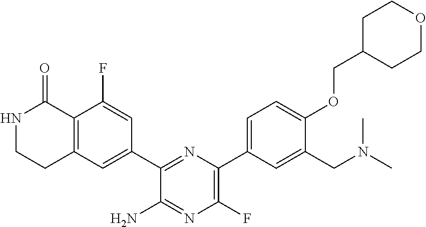 [SMILES: CN(C)Cc1cc(-c2nc(-c3cc(F)c4c(c3)CCNC4=O)c(N)nc2F)ccc1OCC1CCOCC1] | A | |
| I-181 | 6-(3-amino-5-fluoro-6- (4-(tetrahydro-2H- pyran-4- yl)phenyl)pyrazin-2-yl)- 4-methylisoquinolin- 1(2H)-one | 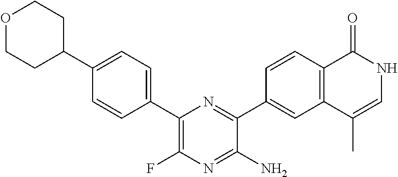 [SMILES: Cc1c[nH]c(=O)c2ccc(-c3nc(-c4ccc(C5CCOCC5)cc4)c(F)nc3N)cc12] | A | |
| I-182 | 6-(3-amino-5-fluoro-6- (4-(4- isopropylpiperazin-1- yl)phenyl)pyrazin-2-yl)- 7-fluoro-3- methylisoquinolin- 1(2H)-one | 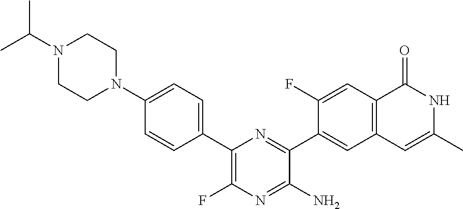 [SMILES: Cc1cc2cc(-c3nc(-c4ccc(N5CCN(C(C)C)CC5)cc4)c(F)nc3N)c(F)cc2c(=O)[nH]1] | A | |
| I-183 | 6-(3-amino-6-(3- ((dimethylamino)methyl)- 4-morpholinophenyl)- 5-fluoropyrazin-2-yl)-7- fluoro-3- methylisoquinolin- 1(2H)-one | 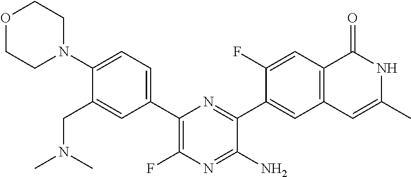 [SMILES: Cc1cc2cc(-c3nc(-c4ccc(N5CCOCC5)c(CN(C)C)c4)c(F)nc3N)c(F)cc2c(=O)[nH]1] | A | |
| I-184 | 6-(3-amino-6-(3- ((dimethylamino)methyl)- 4-(tetrahydro-2H- pyran-4-yl)phenyl)-5- fluoropyrazin-2-yl)-7- fluoro-3- methylisoquinolin- 1(2H)-one | 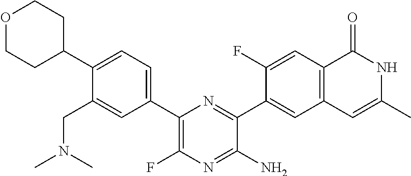 [SMILES: Cc1cc2cc(-c3nc(-c4ccc(C5CCOCC5)c(CN(C)C)c4)c(F)nc3N)c(F)cc2c(=O)[nH]1] | A | |
| I-185 | 6-(3-amino-5-fluoro-6- (4-morpholino-3- (piperidin-1- ylmethyl)phenyl)pyrazin- 2-yl)-3,4- dihydroisoquinolin- 1(2H)-one | 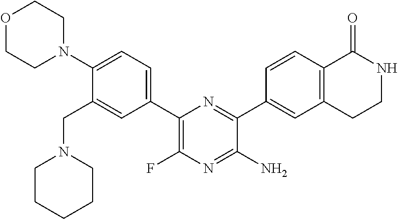 [SMILES: Nc1nc(F)c(-c2ccc(N3CCOCC3)c(CN3CCCCC3)c2)nc1-c1ccc2c(c1)CCNC2=O] | B | |
| I-186 | 6-(3-amino-5-fluoro-6- (4-morpholino-3- (piperidin-1- ylmethyl)phenyl)pyrazin- 2-yl)-7-fluoro-3,4- dihydroisoquinolin- 1(2H)-one | 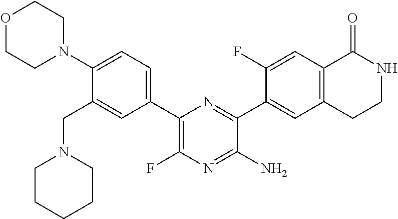 [SMILES: Nc1nc(F)c(-c2ccc(N3CCOCC3)c(CN3CCCCC3)c2)nc1-c1cc2c(cc1F)C(=O)NCC2] | B | |
| I-187 | 6-(3-amino-5-fluoro-6- (4-morpholino-3- (piperidin-1- ylmethyl)phenyl)pyrazin- 2-yl)-8-fluoro-3,4- dihydroisoquinolin- 1(2H)-one | 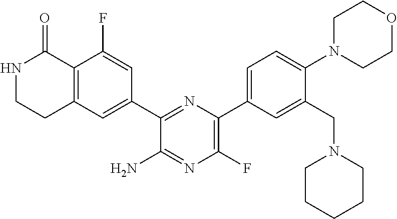 [SMILES: Nc1nc(F)c(-c2ccc(N3CCOCC3)c(CN3CCCCC3)c2)nc1-c1cc(F)c2c(c1)CCNC2=O] | B | |
| I-188 | 6-(3-amino-5-fluoro-6- (3-((3-methoxyazetidin- 1-yl)methyl)-4- morpholinophenyl) pyrazin- 2-yl)-3,4- dihydroisoquinolin- 1(2H)-one | 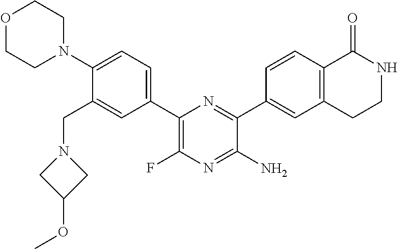 [SMILES: COC1CN(Cc2cc(-c3nc(-c4ccc5c(c4)CCNC5=O)c(N)nc3F)ccc2N2CCOCC2)C1] | A | |
| I-189 | 6-(3-amino-5-fluoro-6- (3-((3-methoxyazetidin- 1-yl)methyl)-4- morpholinophenyl) pyrazin-2-yl)- 7-fluoro-3,4- dihydroisoquinolin- 1(2H)-one | 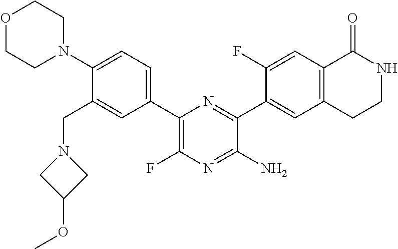 [SMILES: COC1CN(Cc2cc(-c3nc(-c4cc5c(cc4F)C(=O)NCC5)c(N)nc3F)ccc2N2CCOCC2)C1] | A | |
| I-190 | 6-(3-amino-5-fluoro-6- (3-((3-methoxyazetidin- 1-yl)methyl)-4- (tetrahydro-2H-pyran-4- yl)phenyl)pyrazin-2-yl)- 7-fluoro-3,4- dihydroisoquinolin- 1(2H)-one | 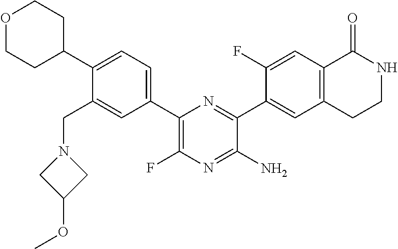 [SMILES: COC1CN(Cc2cc(-c3nc(-c4cc5c(cc4F)C(=O)NCC5)c(N)nc3F)ccc2C2CCOCC2)C1] | A | |
| I-191 | 6-(3-amino-5-fluoro-6- (3-((3-methoxyazetidin- 1-yl)methyl)-4- (tetrahydro-2H-pyran-4- yl)phenyl)pyrazin-2-yl)- 8-fluoro-3,4- dihydroisoquinolin- 1(2H)-one | 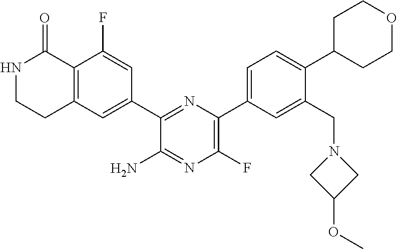 [SMILES: COC1CN(Cc2cc(-c3nc(-c4cc(F)c5c(c4)CCNC5=O)c(N)nc3F)ccc2C2CCOCC2)C1] | A | |
| I-192 | 6-(3-amino-5-fluoro-6- (3-((3-methoxyazetidin- 1-yl)methyl)-4- morpholinophenyl) pyrazin-2-yl)-8-fluoro-3,4- dihydroisoquinolin- 1(2H)-one | 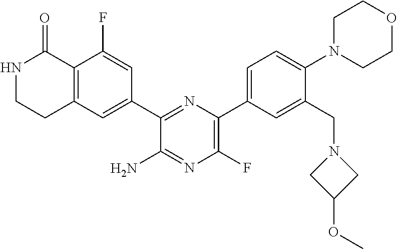 [SMILES: COC1CN(Cc2cc(-c3nc(-c4cc(F)c5c(c4)CCNC5=O)c(N)nc3F)ccc2N2CCOCC2)C1] | A | |
| I-193 | 6-(3-amino-5-fluoro-6- (3-((3-methoxyazetidin- 1-yl)methyl)-4- (tetrahydro-2H-pyran-4- yl)phenyl)pyrazin-2-yl)- 3,4-dihydroisoquinolin- 1(2H)-one | 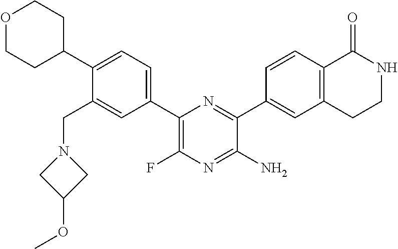 [SMILES: COC1CN(Cc2cc(-c3nc(-c4ccc5c(c4)CCNC5=O)c(N)nc3F)ccc2C2CCOCC2)C1] | A | |
| I-194 | 7-(3-amino-6-(3- ((dimethylamino)methyl)- 4-((2R,6S)-2,6- dimethylmorpholino) phenyl)-5-fluoropyrazin- 2-yl)-2-methylquinazolin- 4(3H)-one | 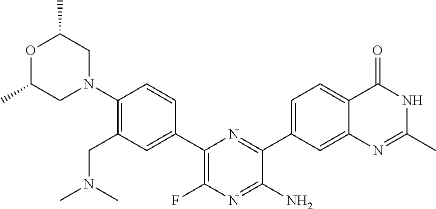 | B | |
| I-195 | 6-(3-amino-6-(4- ((1S,5R)-3-cyclobutyl-3- azabicyclo[3.1.0]hexan- 1-yl)phenyl)-5- fluoropyrazin-2-yl)- 4,4,8-trifluoro-3-methyl- 3,4-dihydroisoquinolin- 1(2H)-one | 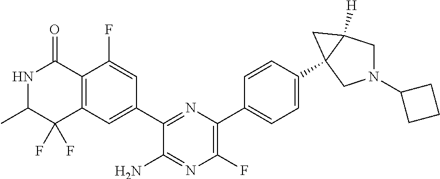 [SMILES: CC1NC(=O)c2c(F)cc(-c3nc(-c4ccc([C@]56C[C@H]5CN(C5CCC5)C6)cc4)c(F)nc3N)cc2C1(F)F] | B | |
| I-196 | 7-(3-amino-6-(3- ((dimethylamino)methyl)- 4-((2R,6S)-2,6- dimethylmorpholino) phenyl)-5-fluoropyrazin-2- yl)-5-fluoro-2- methylquinazolin- 4(3H)-one | 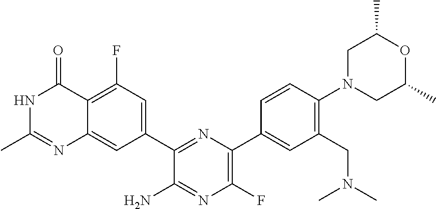 | B | |
| I-197 | 6-(3-amino-6-(3- ((dimethylamino)methyl)- 4-((2R,6S)-2,6- dimethylmorpholino) phenyl)-5- fluoropyrazin-2- yl)-8-fluoro-3,4- dihydroisoquinolin- 1(2H)-one | 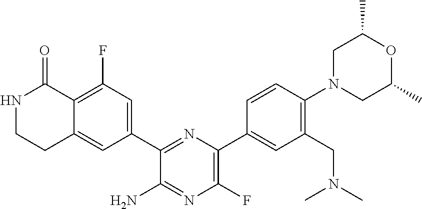 [SMILES: C[C@@H]1CN(c2ccc(-c3nc(-c4cc(F)c5c(c4)CCNC5=O)c(N)nc3F)cc2CN(C)C)C[C@H](C)O1] | A | |
| I-198 | 6-(3-amino-6-(3- ((dimethylamino)methyl)- 4-((2R,6S)-2,6- dimethylmorpholino) phenyl)-5-fluoropyrazin-2- yl)-3,4- dihydroisoquinolin- 1(2H)-one | 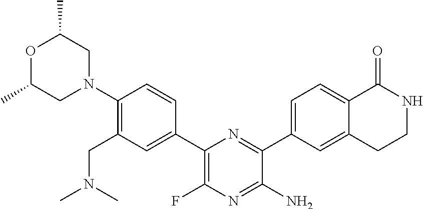 [SMILES: C[C@@H]1CN(c2ccc(-c3nc(-c4ccc5c(c4)CCNC5=O)c(N)nc3F)cc2CN(C)C)C[C@H](C)O1] | A | |
| I-199 | 6-(3-amino-6-(3- (azetidin-1-ylmethyl)-4- morpholinophenyl)-5- fluoropyrazin-2-yl)-4- fluoroisoquinolin-1(2H)- one | 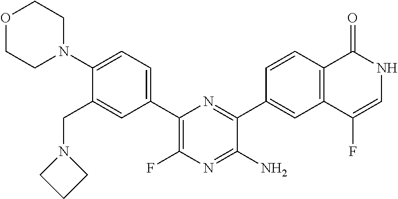 [SMILES: Nc1nc(F)c(-c2ccc(N3CCOCC3)c(CN3CCC3)c2)nc1-c1ccc2c(=O)[nH]cc(F)c2c1] | A | |
| I-200 | 6-(3-amino-5-fluoro-6- (4-morpholino-3- (pyrrolidin-1- ylmethyl)phenyl)pyrazin- 2-yl)-4- fluoroisoquinolin-1(2H)- one | 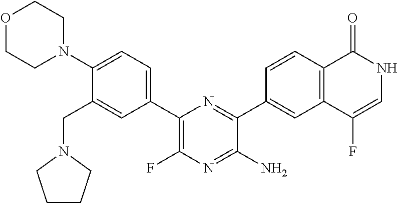 [SMILES: Nc1nc(F)c(-c2ccc(N3CCOCC3)c(CN3CCCC3)c2)nc1-c1ccc2c(=O)[nH]cc(F)c2c1] | A | |
| I-201 | 6-(3-amino-5-fluoro-6- (5-morpholinopyridin-2- yl)pyrazin-2-yl)-3,4- dihydroisoquinolin- 1(2H)-one | 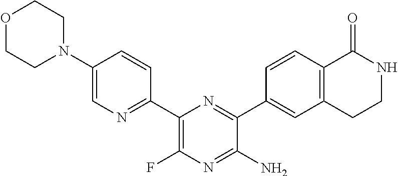 [SMILES: Nc1nc(F)c(-c2ccc(N3CCOCC3)cn2)nc1-c1ccc2c(c1)CCNC2=O] | B | |
| I-202 | 6-(3-amino-6-(3- (azetidin-1-ylmethyl)-4- morpholinophenyl)-5- fluoropyrazin-2-yl)-4- methylisoquinolin- 1(2H)-one | 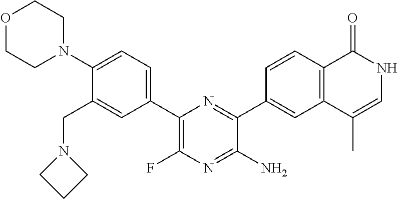 [SMILES: Cc1c[nH]c(=O)c2ccc(-c3nc(-c4ccc(N5CCOCC5)c(CN5CCC5)c4)c(F)nc3N)cc12] | A | |
| I-203 | 6-(3-amino-5-fluoro-6- (4-morpholino-3- (pyrrolidin-1- ylmethyl)phenyl)pyrazin- 2-yl)-4- methylisoquinolin- 1(2H)-one | 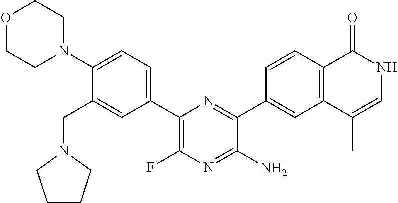 [SMILES: Cc1c[nH]c(=O)c2ccc(-c3nc(-c4ccc(N5CCOCC5)c(CN5CCCC5)c4)c(F)nc3N)cc12] | A | |
| I-204 | 6-(3-amino-6-(3- (azetidin-1-ylmethyl)-4- (tetrahydro-2H-pyran-4- yl)phenyl)-5- fluoropyrazin-2-yl)-4- fluoroisoquinolin-1(2H)- one | 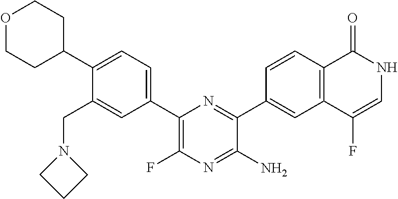 [SMILES: Nc1nc(F)c(-c2ccc(C3CCOCC3)c(CN3CCC3)c2)nc1-c1ccc2c(=O)[nH]cc(F)c2c1] | A | |
| I-205 | 6-(3-amino-5-fluoro-6- (3-(pyrrolidin-1- ylmethyl)-4-(tetrahydro- 2H-pyran-4- yl)phenyl)pyrazin-2-yl)- 4-methylisoquinolin- 1(2H)-one | 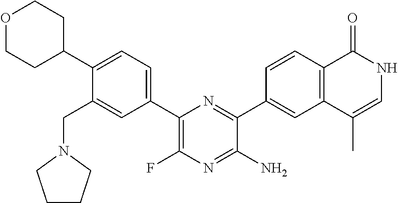 [SMILES: Cc1c[nH]c(=O)c2ccc(-c3nc(-c4ccc(C5CCOCC5)c(CN5CCCC5)c4)c(F)nc3N)cc12] | A | |
| I-206 | 6-(3-amino-6-(3- (azetidin-1-ylmethyl)-4- (tetrahydro-2H-pyran-4- yl)phenyl)-5- fluoropyrazin-2-yl)-4- methylisoquinolin- 1(2H)-one | 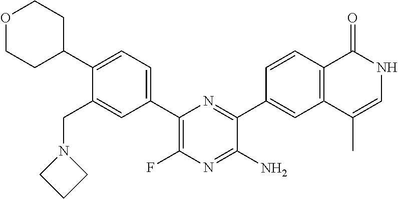 [SMILES: Cc1c[nH]c(=O)c2ccc(-c3nc(-c4ccc(C5CCOCC5)c(CN5CCC5)c4)c(F)nc3N)cc12] | A | |
| I-207 | 6-(3-amino-5-fluoro-6- (3-(pyrrolidin-1- ylmethyl)-4-(tetrahydro- 2H-pyran-4- yl)phenyl)pyrazin-2-yl)- 4-fluoroisoquinolin- 1(2H)-one | 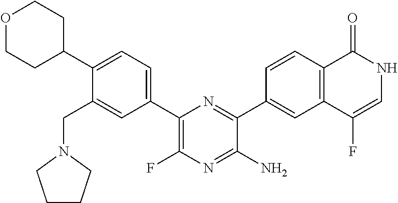 [SMILES: Nc1nc(F)c(-c2ccc(C3CCOCC3)c(CN3CCCC3)c2)nc1-c1ccc2c(=O)[nH]cc(F)c2c1] | A | |
| I-208 | 6-(3-amino-6-(3- (azetidin-1-ylmethyl)-4- ((tetrahydro-2H-pyran- 4-yl)oxy)phenyl)-5- fluoropyrazin-2-yl)-3,4- dihydroisoquinolin- 1(2H)-one | 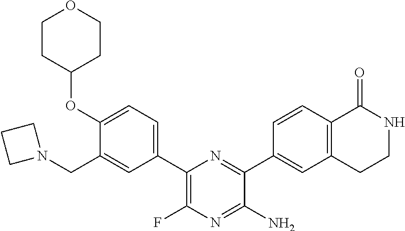 [SMILES: Nc1nc(F)c(-c2ccc(OC3CCOCC3)c(CN3CCC3)c2)nc1-c1ccc2c(c1)CCNC2=O] | B | |
| I-209 | 6-(3-amino-6-(3- (azetidin-1-ylmethyl)-4- ((tetrahydro-2H-pyran- 4-yl)oxy)phenyl)-5- fluoropyrazin-2-yl)-7- fluoro-3,4- dihydroisoquinolin- 1(2H)-one | 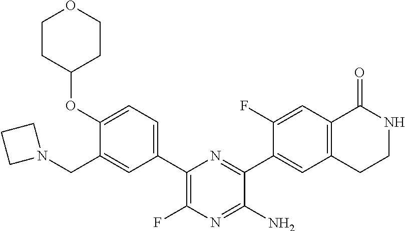 [SMILES: Nc1nc(F)c(-c2ccc(OC3CCOCC3)c(CN3CCC3)c2)nc1-c1cc2c(cc1F)C(=O)NCC2] | B | |
| I-210 | 6-(3-amino-6-(3- (azetidin-1-ylmethyl)-4- ((tetrahydro-2H-pyran- 4-yl)oxy)phenyl)-5- fluoropyrazin-2-yl)-8- fluoro-3,4- dihydroisoquinolin- 1(2H)-one | 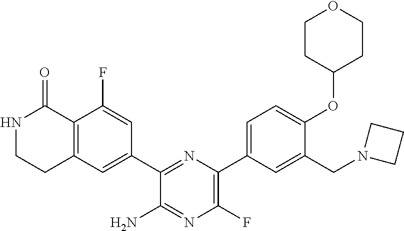 [SMILES: Nc1nc(F)c(-c2ccc(OC3CCOCC3)c(CN3CCC3)c2)nc1-c1cc(F)c2c(c1)CCNC2=O] | B | |
| I-211 | (S)-6-(3-amino-6-(3- ((dimethylamino) methyl)-4-(2- methylmorpholino) phenyl)-5-fluoropyrazin- 2-yl)-3,4- dihydroisoquinolin- 1(2H)-one | 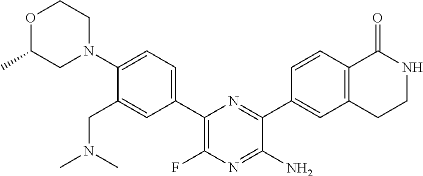 [SMILES: C[C@H]1CN(c2ccc(-c3nc(-c4ccc5c(c4)CCNC5=O)c(N)nc3F)cc2CN(C)C)CCO1] | A | |
| I-212 | (S)-6-(3-amino-6-(3- ((dimethylamino) methyl)-4-(2- methylmorpholino) phenyl)-5-fluoropyrazin- 2-yl)-7-fluoro-3,4- dihydroisoquinolin- 1(2H)-one | 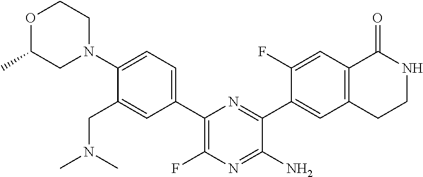 [SMILES: C[C@H]1CN(c2ccc(-c3nc(-c4cc5c(cc4F)C(=O)NCC5)c(N)nc3F)cc2CN(C)C)CCO1] | A | |
| I-213 | (S)-6-(3-amino-6-(3- ((dimethylamino) methyl)-4-(2- methylmorpholino) phenyl)-5-fluoropyrazin- 2-yl)-8-fluoro-3,4- dihydroisoquinolin- 1(2H)-one | 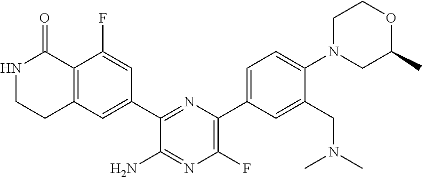 [SMILES: C[C@H]1CN(c2ccc(-c3nc(-c4cc(F)c5c(c4)CCNC5=O)c(N)nc3F)cc2CN(C)C)CCO1] | A | |
| I-214 | (S)-6-(3-amino-6-(3- (azetidin-1- ylmethyl)-4-(2- methylmorpholino) phenyl)-5-fluoropyrazin- 2-yl)-3,4- dihydroisoquinolin- 1(2H)-one | 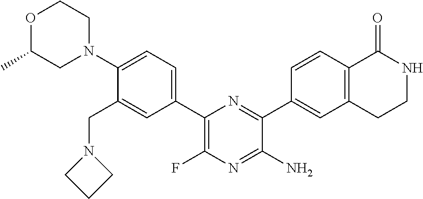 [SMILES: C[C@H]1CN(c2ccc(-c3nc(-c4ccc5c(c4)CCNC5=O)c(N)nc3F)cc2CN2CCC2)CCO1] | A | |
| I-215 | (S)-6-(3-amino-6-(3- (azetidin-1-ylmethyl)-4- (2-methylmorpholino) phenyl)-5- fluoropyrazin-2- yl)-7-fluoro-3,4- dihydroisoquinolin- 1(2H)-one | 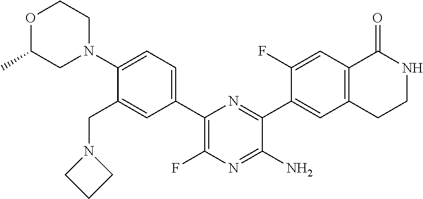 [SMILES: C[C@H]1CN(c2ccc(-c3nc(-c4cc5c(cc4F)C(=O)NCC5)c(N)nc3F)cc2CN2CCC2)CCO1] | A | |
| I-216 | (S)-6-(3-amino-6-(3- (azetidin-1-ylmethyl)-4- (2-methylmorpholino) phenyl)-5- fluoropyrazin-2- yl)-8-fluoro-3,4- dihydroisoquinolin- 1(2H)-one | 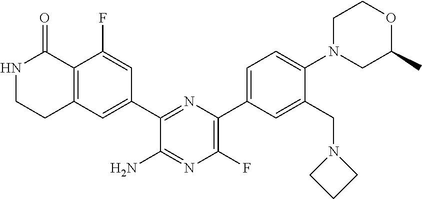 [SMILES: C[C@H]1CN(c2ccc(-c3nc(-c4cc(F)c5c(c4)CCNC5=O)c(N)nc3F)cc2CN2CCC2)CCO1] | B | |
| I-217 | (R)-7-(3-amino-5- fluoro-6-(3-((2- methylpyrrolidin-1- yl)methyl)-4- (tetrahydro-2H-pyran-4- yl)phenyl)pyrazin-2-yl)- 5-fluoro-2- methylquinazolin- 4(3H)-one | 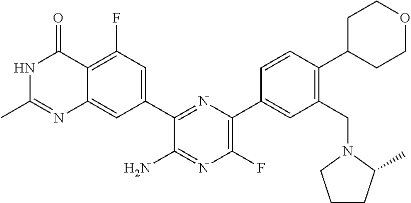 [SMILES: Cc1nc2cc(-c3nc(-c4ccc(C5CCOCC5)c(CN5CCC[C@H]5C)c4)c(F)nc3N)cc(F)c2c(=O)[nH]1] | B | |
| I-218 | (R)-6-(3-amino-5- fluoro-6-(3-((2- methylpyrrolidin-1- yl)methyl)-4- (tetrahydro-2H-pyran-4- yl)phenyl)pyrazin-2-yl)- 8-fluoro-3,4- dihydroisoquinolin- 1(2H)-one | 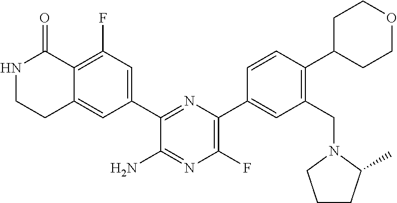 [SMILES: C[C@@H]1CCCN1Cc1cc(-c2nc(-c3cc(F)c4c(c3)CCNC4=O)c(N)nc2F)ccc1C1CCOCC1] | B | |
| I-219 | (S)-7-(3-amino-5-fluoro- 6-(3-((2- methylpyrrolidin-1- yl)methyl)-4- (tetrahydro-2H-pyran-4- yl)phenyl)pyrazin-2-yl)- 5-fluoro-2- methylquinazolin- 4(3H)-one | 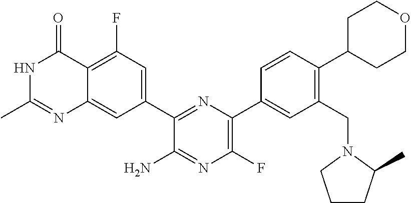 [SMILES: Cc1nc2cc(-c3nc(-c4ccc(C5CCOCC5)c(CN5CCC[C@@H]5C)c4)c(F)nc3N)cc(F)c2c(=O)[nH]1] | C | |
| I-220 | (S)-6-(3-amino-5-fluoro- 6-(3-((2- methylpyrrolidin-1- yl)methyl)-4- (tetrahydro-2H-pyran-4- yl)phenyl)pyrazin-2-yl)- 8-fluoro-3,4- dihydroisoquinolin- 1(2H)-one | 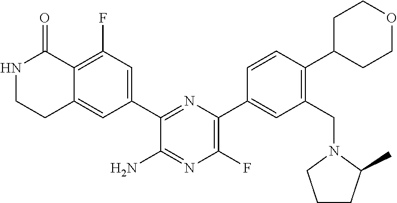 [SMILES: C[C@H]1CCCN1Cc1cc(-c2nc(-c3cc(F)c4c(c3)CCNC4=O)c(N)nc2F)ccc1C1CCOCC1] | B | |
| I-221 | 6-(3-amino-5-fluoro-6- (3-methyl-4-(tetrahydro- 2H-pyran-4- yl)phenyl)pyrazin-2-yl)- 8-fluoro-3,4- dihydroisoquinolin- 1(2H)-one | 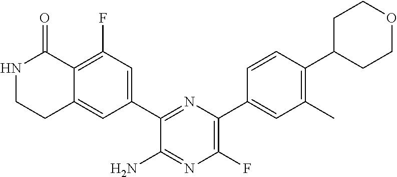 [SMILES: Cc1cc(-c2nc(-c3cc(F)c4c(c3)CCNC4=O)c(N)nc2F)ccc1C1CCOCC1] | A | |
| I-222 | 6-(3-amino-5-fluoro-6- (3-methyl-4-(tetrahydro- 2H-pyran-4- yl)phenyl)pyrazin-2-yl)- 7-fluoro-3,4- dihydroisoquinolin- 1(2H)-one | 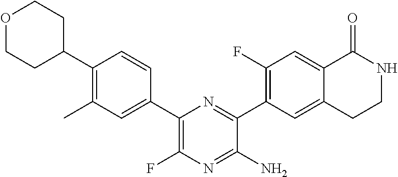 [SMILES: Cc1cc(-c2nc(-c3cc4c(cc3F)C(=O)NCC4)c(N)nc2F)ccc1C1CCOCC1] | A | |
| I-223 | 6-(3-amino-6-(3- ((dimethylamino)methyl)- 4-(tetrahydro-2H- pyran-4-yl)phenyl)-5- fluoropyrazin-2-yl)-4,4- difluoro-3,4- dihydroisoquinolin- 1(2H)-one | 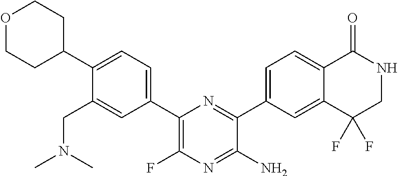 [SMILES: CN(C)Cc1cc(-c2nc(-c3ccc4c(c3)C(F)(F)CNC4=O)c(N)nc2F)ccc1C1CCOCC1] | A | D |
| I-224 | 6-(3-amino-6-(3- ((ethyl(methyl)amino) methyl)-4-(tetrahydro-2H- pyran-4-yl)phenyl)-5- fluoropyrazin-2-yl)-4,4- difluoro-3,4- dihydroisoquinolin- 1(2H)-one | 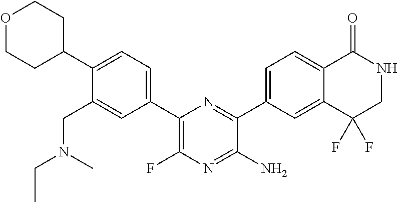 [SMILES: CCN(C)Cc1cc(-c2nc(-c3ccc4c(c3)C(F)(F)CNC4=O)c(N)nc2F)ccc1C1CCOCC1] | A | |
| I-225 | 6-(3-amino-6-(4-(3,6- dihydro-2H-pyran-4-yl)- 3- ((ethyl(methyl)amino) methyl)phenyl)-5- fluoropyrazin-2-yl)-4,4- difluoro-3,4- dihydroisoquinolin- 1(2H)-one | 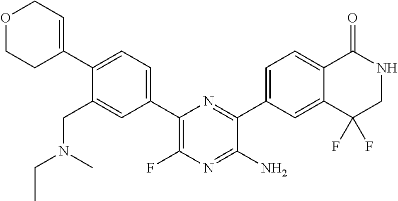 [SMILES: CCN(C)Cc1cc(-c2nc(-c3ccc4c(c3)C(F)(F)CNC4=O)c(N)nc2F)ccc1C1=CCOCC1] | B | |
| I-226 | (R)-6-(3-amino-5- fluoro-6-(3-((2- methylpyrrolidin-1- yl)methyl)-4- (tetrahydro-2H-pyran-4- yl)phenyl)pyrazin-2-yl)- 4-fluoroisoquinolin- 1(2H)-one | 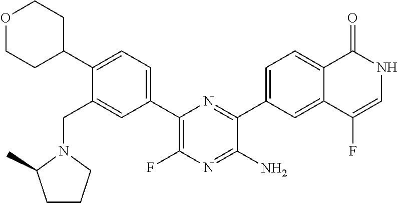 [SMILES: C[C@@H]1CCCN1Cc1cc(-c2nc(-c3ccc4c(=O)[nH]cc(F)c4c3)c(N)nc2F)ccc1C1CCOCC1] | A | |
| I-227 | (R)-6-(3-amino-5- fluoro-6-(3-((2- methylpyrrolidin-1- yl)methyl)-4- (tetrahydro-2H-pyran-4- yl)phenyl)pyrazin-2-yl)- 4-chloroisoquinolin- 1(2H)-one | 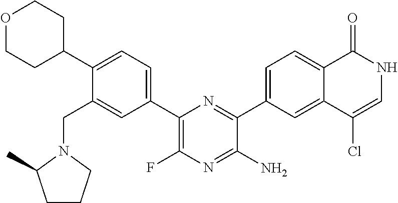 [SMILES: C[C@@H]1CCCN1Cc1cc(-c2nc(-c3ccc4c(=O)[nH]cc(Cl)c4c3)c(N)nc2F)ccc1C1CCOCC1] | B | |
| I-228 | (S)-6-(3-amino-5-fluoro- 6-(3-((2- methylpyrrolidin-1- yl)methyl)-4- (tetrahydro-2H-pyran-4- yl)phenyl)pyrazin-2-yl)- 4-fluoroisoquinolin- 1(2H)-one | 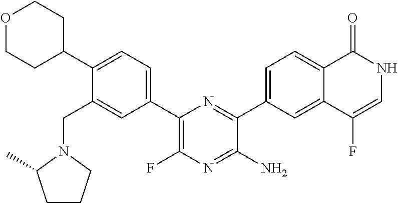 [SMILES: C[C@H]1CCCN1Cc1cc(-c2nc(-c3ccc4c(=O)[nH]cc(F)c4c3)c(N)nc2F)ccc1C1CCOCC1] | A | |
| I-229 | (S)-6-(3-amino-5-fluoro- 6-(3-((2- methylpyrrolidin-1- yl)methyl)-4- (tetrahydro-2H-pyran-4- yl)phenyl)pyrazin-2-yl)- 4-chloroisoquinolin- 1(2H)-one | 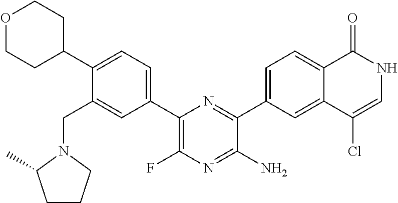 [SMILES: C[C@H]1CCCN1Cc1cc(-c2nc(-c3ccc4c(=O)[nH]cc(Cl)c4c3)c(N)nc2F)ccc1C1CCOCC1] | B | |
| I-230 | 6-(3-amino-6-(3- ((dimethylamino)methyl)- 4-((2R,6S)-2,6- dimethylmorpholino) phenyl)-5-fluoropyrazin-2- yl)-4-fluoroisoquinolin- 1(2H)-one | 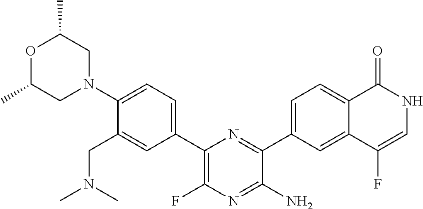 [SMILES: C[C@@H]1CN(c2ccc(-c3nc(-c4ccc5c(=O)[nH]cc(F)c5c4)c(N)nc3F)cc2CN(C)C)C[C@H](C)O1] | A | |
| I-231 | 6-(3-amino-6-(3- ((dimethylamino)methyl)- 4-((2R,6S)-2,6- dimethylmorpholino) phenyl)-5-fluoropyrazin-2- yl)-4-chloroisoquinolin- 1(2H)-one | 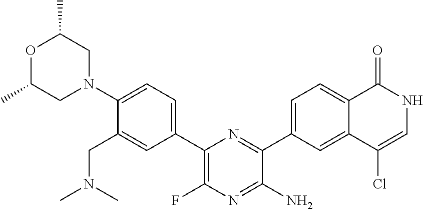 [SMILES: C[C@@H]1CN(c2ccc(-c3nc(-c4ccc5c(=O)[nH]cc(Cl)c5c4)c(N)nc3F)cc2CN(C)C)C[C@H](C)O1] | A | |
| I-232 | (R)-6-(3-amino-5- fluoro-6-(3-((2- methylpyrrolidin-1- yl)methyl)-4- (tetrahydro-2H-pyran-4- yl)phenyl)pyrazin-2-yl)- 4,4-difluoro-3,4- dihydroisoquinolin- 1(2H)-one | 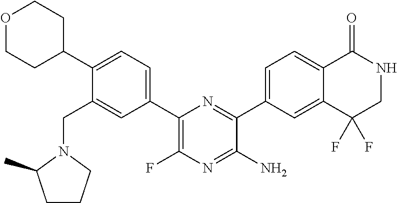 [SMILES: C[C@@H]1CCCN1Cc1cc(-c2nc(-c3ccc4c(c3)C(F)(F)CNC4=O)c(N)nc2F)ccc1C1CCOCC1] | A | |
| I-233 | (S)-6-(3-amino-5-fluoro- 6-(3-((2- methylpyrrolidin-1- yl)methyl)-4- (tetrahydro-2H-pyran-4- yl)phenyl)pyrazin-2-yl)- 4,4-difluoro-3,4- dihydroisoquinolin- 1(2H)-one | 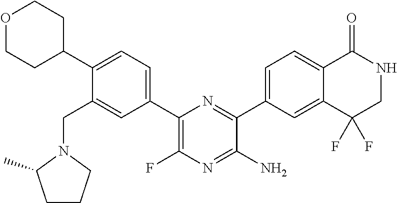 [SMILES: C[C@H]1CCCN1Cc1cc(-c2nc(-c3ccc4c(c3)C(F)(F)CNC4=O)c(N)nc2F)ccc1C1CCOCC1] | A | |
| I-234 | 6-(3-amino-5-fluoro-6- (4-(4- isopropylpiperazin-1- yl)phenyl)pyrazin-2-yl)- 7-fluoro-4- methylisoquinolin- 1(2H)-one | 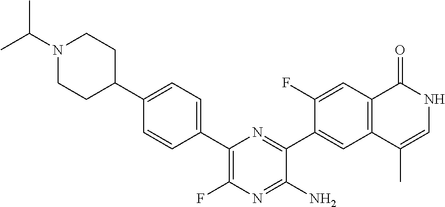 [SMILES: Cc1c[nH]c(=O)c2cc(F)c(-c3nc(-c4ccc(C5CCN(C(C)C)CC5)cc4)c(F)nc3N)cc12] | A | |
| I-235 | 6-(3-amino-6-(3- ((dimethylamino)methyl)- 4-(tetrahydro-2H- pyran-4-yl)phenyl)-5- fluoropyrazin-2-yl)-7- fluoro-4- methylisoquinolin- 1(2H)-one | 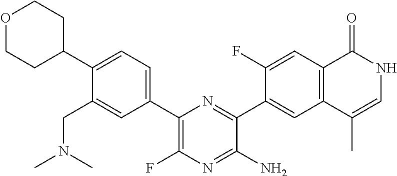 [SMILES: Cc1c[nH]c(=O)c2cc(F)c(-c3nc(-c4ccc(C5CCOCC5)c(CN(C)C)c4)c(F)nc3N)cc12] | A | |
| I-236 | 6-(3-amino-6-(5- ((dimethylamino)methyl)- 6-morpholinopyridin- 3-yl)-5-fluoropyrazin-2- yl)-4-methylisoquinolin- 1(2H)-one | 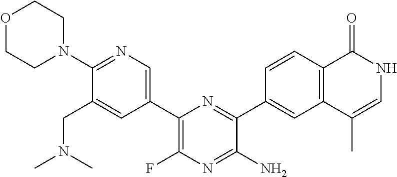 [SMILES: Cc1c[nH]c(=O)c2ccc(-c3nc(-c4cnc(N5CCOCC5)c(CN(C)C)c4)c(F)nc3N)cc12] | B | |
| I-237 | 6-(3-amino-6-(5- ((dimethylamino)methyl)- 6-morpholinopyridin- 3-yl)-5-fluoropyrazin-2- yl)-3,4- dihydroisoquinolin- 1(2H)-one | 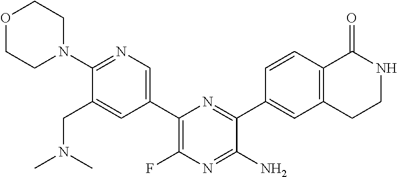 [SMILES: CN(C)Cc1cc(-c2nc(-c3ccc4c(c3)CCNC4=O)c(N)nc2F)cnc1N1CCOCC1] | B | |
| I-238 | 6-(3-amino-6-(3- ((ethyl(methyl)amino) methyl)-4-(tetrahydro-2H- pyran-4-yl)phenyl)-5- fluoropyrazin-2-yl)-4- fluoroisoquinolin-1(2H)- one | 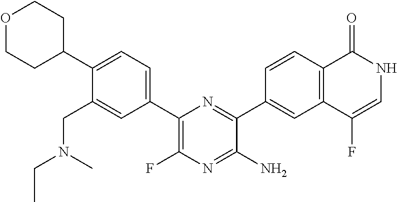 [SMILES: CCN(C)Cc1cc(-c2nc(-c3ccc4c(=O)[nH]cc(F)c4c3)c(N)nc2F)ccc1C1CCOCC1] | A | D |
| I-239 | 6-(3-amino-6-(3- ((ethyl(methyl)amino) methyl)-4-(tetrahydro-2H- pyran-4-yl)phenyl)-5- fluoropyrazin-2-yl)-4- methylisoquinolin- 1(2H)-one | 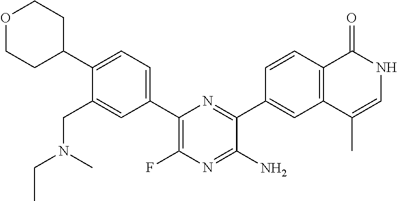 [SMILES: CCN(C)Cc1cc(-c2nc(-c3ccc4c(=O)[nH]cc(C)c4c3)c(N)nc2F)ccc1C1CCOCC1] | A | |
| I-240 | 6-(3-amino-6-(3- ((dimethylamino)methyl)- 2-fluoro-4- morpholinophenyl)-5- fluoropyrazin-2-yl)-3,4- dihydroisoquinolin- 1(2H)-one | 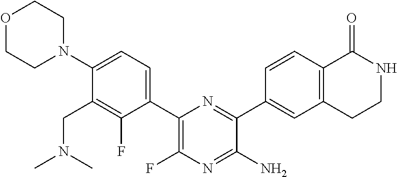 [SMILES: CN(C)Cc1c(N2CCOCC2)ccc(-c2nc(-c3ccc4c(c3)CCNC4=O)c(N)nc2F)c1F] | B | |
| I-241 | 6-(3-amino-6-(3- ((dimethylamino)methyl)- 2-fluoro-4- morpholinophenyl)-5- fluoropyrazin-2-yl)-7- fluoro-3,4- dihydroisoquinolin- 1(2H)-one | 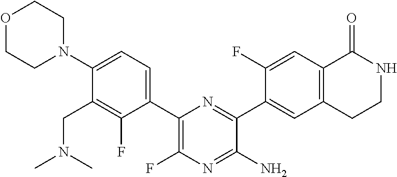 [SMILES: CN(C)Cc1c(N2CCOCC2)ccc(-c2nc(-c3cc4c(cc3F)C(=O)NCC4)c(N)nc2F)c1F] | B | |
| I-242 | 6-(3-amino-6-(3- ((dimethylamino)methyl)- 2-fluoro-4- morpholinophenyl)-5- fluoropyrazin-2-yl)-8- fluoro-3,4- dihydroisoquinolin- 1(2H)-one | 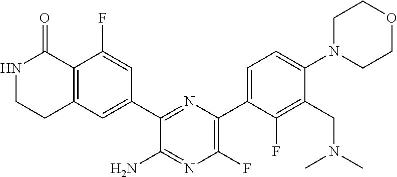 [SMILES: CN(C)Cc1c(N2CCOCC2)ccc(-c2nc(-c3cc(F)c4c(c3)CCNC4=O)c(N)nc2F)c1F] | B | |
| I-243 | 6-(3-amino-6-(3- (azetidin-1-ylmethyl)-2- fluoro-4- morpholinophenyl)-5- fluoropyrazin-2-yl)-3,4- dihydroisoquinolin- 1(2H)-one | 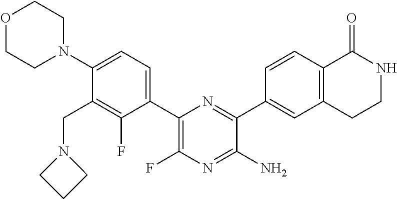 [SMILES: Nc1nc(F)c(-c2ccc(N3CCOCC3)c(CN3CCC3)c2F)nc1-c1ccc2c(c1)CCNC2=O] | B | |
| I-244 | 6-(3-amino-6-(3- (azetidin-1-ylmethyl)-2- fluoro-4- morpholinophenyl)-5- fluoropyrazin-2-yl)-7- fluoro-3,4- dihydroisoquinolin- 1(2H)-one | 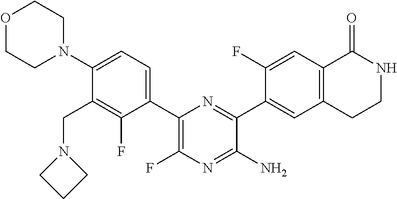 [SMILES: Nc1nc(F)c(-c2ccc(N3CCOCC3)c(CN3CCC3)c2F)nc1-c1cc2c(cc1F)C(=O)NCC2] | B | |
| I-245 | 6-(3-amino-6-(3- (azetidin-1-ylmethyl)-2- fluoro-4- morpholinophenyl)-5- fluoropyrazin-2-yl)-8- fluoro-3,4- dihydroisoquinolin- 1(2H)-one | 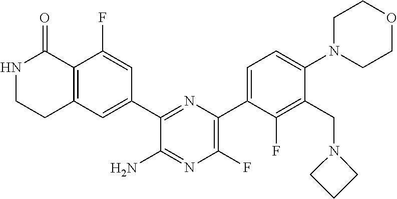 [SMILES: Nc1nc(F)c(-c2ccc(N3CCOCC3)c(CN3CCC3)c2F)nc1-c1cc(F)c2c(c1)CCNC2=O] | B | |
| I-246 | 6-(3-amino-6-(3- ((ethyl(methyl)amino)m ethyl)-2-fluoro-4- morpholinophenyl)-5- fluoropyrazin-2-yl)-7- fluoro-3,4- dihydroisoquinolin- 1(2H)-one | 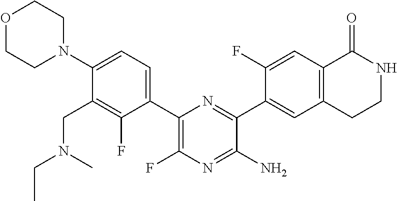 [SMILES: CCN(C)Cc1c(N2CCOCC2)ccc(-c2nc(-c3cc4c(cc3F)C(=O)NCC4)c(N)nc2F)c1F] | B | |
| I-247 | 6-(3-amino-6-(3- ((ethyl(methyl)amino) methyl)-2-fluoro-4- morpholinophenyl)-5- fluoropyrazin-2-yl)-8- fluoro-3,4- dihydroisoquinolin- 1(2H)-one | 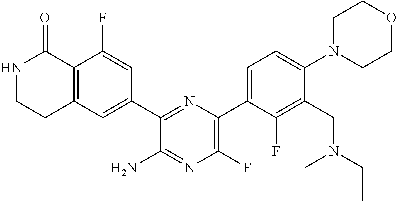 [SMILES: CCN(C)Cc1c(N2CCOCC2)ccc(-c2nc(-c3cc(F)c4c(c3)CCNC4=O)c(N)nc2F)c1F] | B | |
| I-248 | 6-(3-amino-6-(3- ((ethyl(methyl)amino) methyl)-2-fluoro-4- morpholinophenyl)-5- fluoropyrazin-2-yl)-3,4- dihydroisoquinolin- 1(2H)-one | 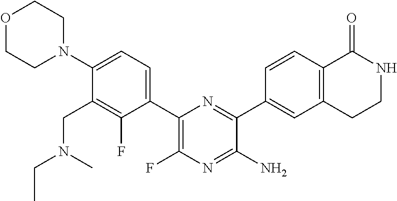 [SMILES: CCN(C)Cc1c(N2CCOCC2)ccc(-c2nc(-c3ccc4c(c3)CCNC4=O)c(N)nc2F)c1F] | B | |
| I-249 | 6-(3-amino-6-(3- ((dimethylamino)methyl)- 4-methoxyphenyl)-5- fluoropyrazin-2-yl)-7- fluoro-3,4- dihydroisoquinolin- 1(2H)-one | 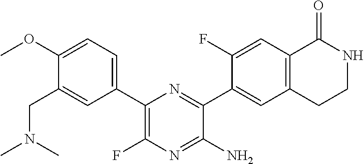 [SMILES: COc1ccc(-c2nc(-c3cc4c(cc3F)C(=O)NCC4)c(N)nc2F)cc1CN(C)C] | A | |
| I-250 | 6-(3-amino-6-(3- ((dimethylamino)methyl)- 4-methoxyphenyl)-5- fluoropyrazin-2-yl)-3,4- dihydroisoquinolin- 1(2H)-one | 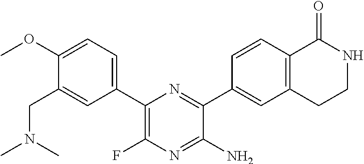 [SMILES: COc1ccc(-c2nc(-c3ccc4c(c3)CCNC4=O)c(N)nc2F)cc1CN(C)C] | B | |
| I-251 | (S)-6-(3-amino-6-(3- (dimethylamino)-2,3- dihydrobenzofuran-5- yl)-5-fluoropyrazin-2- yl)-4-fluoroisoquinolin- 1(2H)-one | 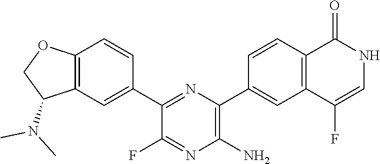 [SMILES: CN(C)[C@@H]1COc2ccc(-c3nc(-c4ccc5c(=O)[nH]cc(F)c5c4)c(N)nc3F)cc21] | A | |
| I-252 | (S)-6-(3-amino-6-(3- (dimethylamino)-2,3- dihydrobenzofuran-5- yl)-5-fluoropyrazin-2- yl)-4-chloroisoquinolin- 1(2H)-one | 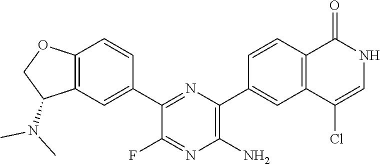 [SMILES: CN(C)[C@@H]1COc2ccc(-c3nc(-c4ccc5c(=O)[nH]cc(Cl)c5c4)c(N)nc3F)cc21] | A | |
| I-253 | (S)-6-(3-amino-6-(3- (dimethylamino)-2,3- dihydrobenzofuran-5- yl)-5-fluoropyrazin-2- yl)-4,8- difluoroisoquinolin- 1(2H)-one | 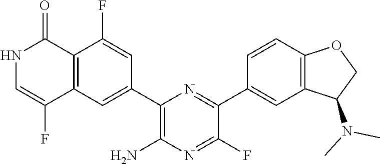 [SMILES: CN(C)[C@@H]1COc2ccc(-c3nc(-c4cc(F)c5c(=O)[nH]cc(F)c5c4)c(N)nc3F)cc21] | A | |
| I-254 | (S)-6-(3-amino-6-(3- (dimethylamino)-2,3- dihydrobenzofuran-5- yl)-5-fluoropyrazin-2- yl)-8-fluoro-3,4- dihydroisoquinolin- 1(2H)-one | 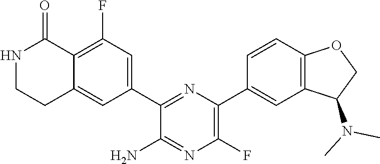 [SMILES: CN(C)[C@@H]1COc2ccc(-c3nc(-c4cc(F)c5c(c4)CCNC5=O)c(N)nc3F)cc21] | B | |
| I-255 | (S)-6-(3-amino-6-(3- (dimethylamino)-2,3- dihydrobenzofuran-5- yl)-5-fluoropyrazin-2- yl)-7-fluoro-3,4- dihydroisoquinolin- 1(2H)-one | 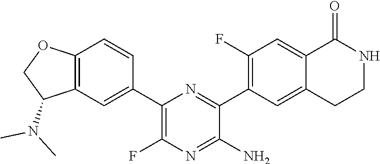 [SMILES: CN(C)[C@@H]1COc2ccc(-c3nc(-c4cc5c(cc4F)C(=O)NCC5)c(N)nc3F)cc21] | B | |
| I-256 | (S)-6-(3-amino-6-(3- (dimethylamino)-2,3- dihydrobenzofuran-5- yl)-5-fluoropyrazin-2- yl)-4,4-difluoro-3,4- dihydroisoquinolin- 1(2H)-one | 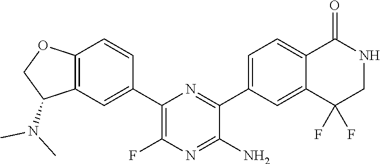 [SMILES: CN(C)[C@@H]1COc2ccc(-c3nc(-c4ccc5c(c4)C(F)(F)CNC5=O)c(N)nc3F)cc21] | A | |
| I-257 | 6-(3-amino-6-(3- ((dimethylamino)methyl)- 4-((2R,6S)-2,6- dimethylmorpholino) phenyl)-5-fluoropyrazin-2- yl)-4,4-difluoro-3,4- dihydroisoquinolin- 1(2H)-one | 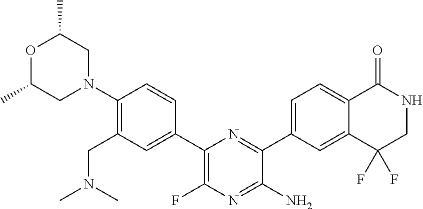 [SMILES: C[C@@H]1CN(c2ccc(-c3nc(-c4ccc5c(c4)C(F)(F)CNC5=O)c(N)nc3F)cc2CN(C)C)C[C@H](C)O1] | A | D |
| I-258 | 6-(3-amino-6-(5- ((dimethylamino)methyl)- 2-fluoro-4- morpholinophenyl)-5- fluoropyrazin-2-yl)-3,4- dihydroisoquinolin- 1(2H)-one | 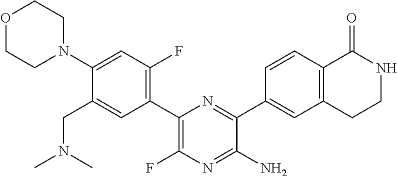 [SMILES: CN(C)Cc1cc(-c2nc(-c3ccc4c(c3)CCNC4=O)c(N)nc2F)c(F)cc1N1CCOCC1] | C | |
| I-259 | 6-(3-amino-6-(5- ((dimethylamino)methyl)- 2-fluoro-4- morpholinophenyl)-5- fluoropyrazin-2-yl)-4- fluoroisoquinolin-1(2H)- one | 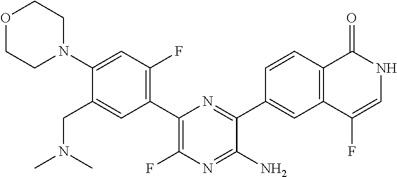 [SMILES: CN(C)Cc1cc(-c2nc(-c3ccc4c(=O)[nH]cc(F)c4c3)c(N)nc2F)c(F)cc1N1CCOCC1] | B | |
| I-260 | 7′-(3-amino-5-fluoro-6- (4-(4- isopropylpiperazin-1- yl)phenyl)pyrazin-2-yl)- 2′, 3′-dihydro-4′H- spiro[cyclopropane- 1, 1′-isoquinolin]-4′-one | 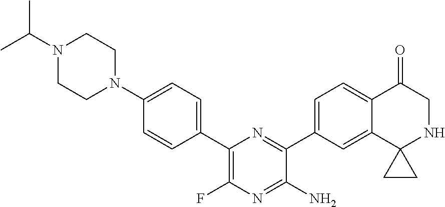 [SMILES: CC(C)N1CCN(c2ccc(-c3nc(-c4ccc5c(c4)C4(CC4)NCC5=O)c(N)nc3F)cc2)CC1] | C | |
| I-261 | 6-(3-amino-6-(5- ((dimethylamino)methyl)- 2-fluoro-4- morpholinophenyl)-5- fluoropyrazin-2-yl)-4- methylisoquinolin- 1(2H)-one | 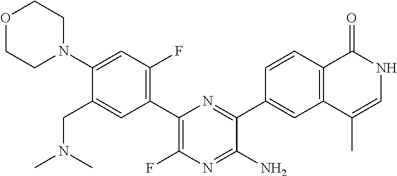 [SMILES: Cc1c[nH]c(=O)c2ccc(-c3nc(-c4cc(CN(C)C)c(N5CCOCC5)cc4F)c(F)nc3N)cc12] | C | |
| I-262 | 6-(3-amino-6-(5- ((dimethylamino) methyl)-2,3-difluoro-4- morpholinophenyl)-5- fluoropyrazin-2-yl)-3,4- dihydroisoquinolin- 1(2H)-one | 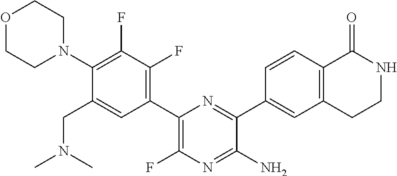 [SMILES: CN(C)Cc1cc(-c2nc(-c3ccc4c(c3)CCNC4=O)c(N)nc2F)c(F)c(F)c1N1CCOCC1] | C | |
| I-263 | 6-(3-amino-6-(5- ((dimethylamino)methyl)- 2,3-difluoro-4- morpholinophenyl)-5- fluoropyrazin-2-yl)-4- fluoroisoquinolin-1(2H)- one | 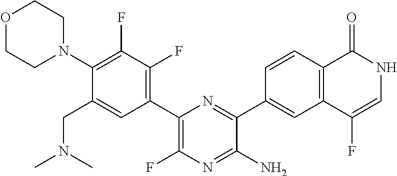 [SMILES: CN(C)Cc1cc(-c2nc(-c3ccc4c(=O)[nH]cc(F)c4c3)c(N)nc2F)c(F)c(F)c1N1CCOCC1] | B | |
| I-264 | 6-(3-amino-6-(5- ((dimethylamino)methyl)- 2,3-difluoro-4- morpholinophenyl)-5- fluoropyrazin-2-yl)-4- methylisoquinolin- 1(2H)-one | 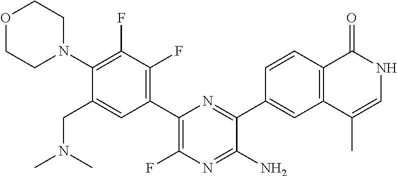 [SMILES: Cc1c[nH]c(=O)c2ccc(-c3nc(-c4cc(CN(C)C)c(N5CCOCC5)c(F)c4F)c(F)nc3N)cc12] | C | |
| I-265 | 6-(3-amino-6-(3- ((dimethylamino)methyl)- 5-fluoro-4- morpholinophenyl)-5- fluoropyrazin-2-yl)-4- fluoroisoquinolin-1(2H)- one | 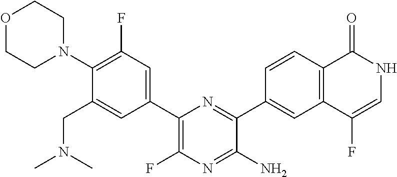 [SMILES: CN(C)Cc1cc(-c2nc(-c3ccc4c(=O)[nH]cc(F)c4c3)c(N)nc2F)cc(F)c1N1CCOCC1] | A | |
| I-266 | 6-(3-amino-6-(3- ((dimethylamino)methyl)- 5-fluoro-4- morpholinophenyl)-5- fluoropyrazin-2-yl)-4- methylisoquinolin- 1(2H)-one | 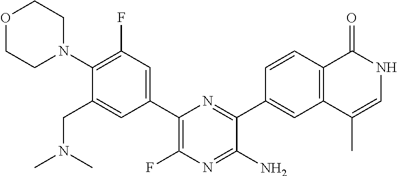 [SMILES: Cc1c[nH]c(=O)c2ccc(-c3nc(-c4cc(F)c(N5CCOCC5)c(CN(C)C)c4)c(F)nc3N)cc12] | A | |
| I-267 | 6-(3-amino-6-(3- ((dimethylamino)methyl)- 4-methoxyphenyl)-5- fluoropyrazin-2-yl)-4- fluoroisoquinolin-1(2H)- one | 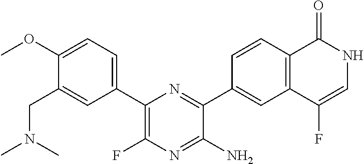 [SMILES: COc1ccc(-c2nc(-c3ccc4c(=O)[nH]cc(F)c4c3)c(N)nc2F)cc1CN(C)C] | A | D |
| I-268 | 6-(3-amino-6-(8- ((dimethylamino)methyl) chroman-6-yl)-5- fluoropyrazin-2-yl)-4- fluoroisoquinolin-1(2H)- one | 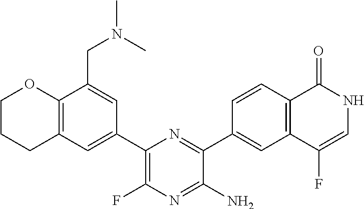 [SMILES: CN(C)Cc1cc(-c2nc(-c3ccc4c(=O)[nH]cc(F)c4c3)c(N)nc2F)cc2c1OCCC2] | A | |
| I-269 | 6-(3-amino-6-(8- ((dimethylamino)methyl)- 2,3- dihydrobenzo[b][1,4] dioxin-6-yl)-5- fluoropyrazin-2-yl)-4- fluoroisoquinolin-1(2H)- one | 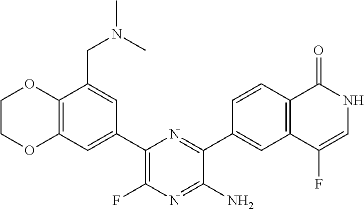 [SMILES: CN(C)Cc1cc(-c2nc(-c3ccc4c(=O)[nH]cc(F)c4c3)c(N)nc2F)cc2c1OCCO2] | A | |
| I-270 | 6-(3-amino-6-(4- (difluoromethoxy)-3- ((dimethylamino)methyl) phenyl)-5- fluoropyrazin-2-yl)-4- fluoroisoquinolin-1(2H)- one | 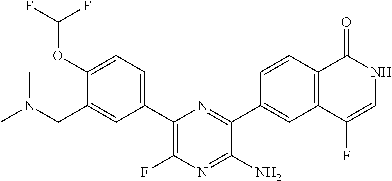 [SMILES: CN(C)Cc1cc(-c2nc(-c3ccc4c(=O)[nH]cc(F)c4c3)c(N)nc2F)ccc1OC(F)F] | A | |
| I-271 | 6-(3-amino-6-(3-(2- aminopropan-2-yl)-4- fluorophenyl)-5- fluoropyrazin-2-yl)-3,4- dihydroisoquinolin- 1(2H)-one | 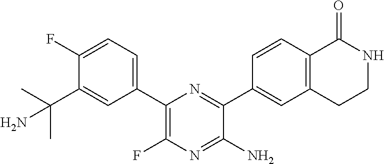 [SMILES: CC(C)(N)c1cc(-c2nc(-c3ccc4c(c3)CCNC4=O)c(N)nc2F)ccc1F] | C | |
| I-272 | 6-(3-amino-6-(3- ((dimethylamino)methyl)- 4-fluorophenyl)-5- fluoropyrazin-2-yl)-3,4- dihydroisoquinolin- 1(2H)-one | 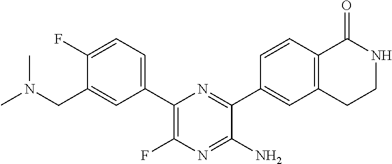 [SMILES: CN(C)Cc1cc(-c2nc(-c3ccc4c(c3)CCNC4=O)c(N)nc2F)ccc1F] | B | |
| I-273 | 6-(3-amino-6-(3- ((dimethylamino)methyl)- 4-fluorophenyl)-5- fluoropyrazin-2-yl)-7- fluoro-3,4- dihydroisoquinolin- 1(2H)-one | 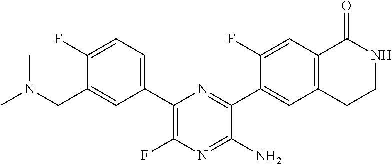 [SMILES: CN(C)Cc1cc(-c2nc(-c3cc4c(cc3F)C(=O)NCC4)c(N)nc2F)ccc1F] | B | |
| I-274 | 6-(3-amino-6-(3- ((dimethylamino)methyl)- 4-fluorophenyl)-5- fluoropyrazin-2-yl)-8- fluoro-3,4- dihydroisoquinolin- 1(2H)-one | 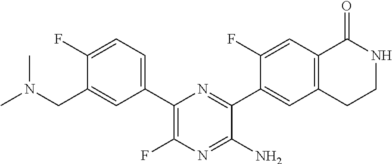 [SMILES: CN(C)Cc1cc(-c2nc(-c3cc4c(cc3F)C(=O)NCC4)c(N)nc2F)ccc1F] | C | |
| I-275 | 6-(3-amino-6-(3- ((dimethylamino)methyl)- 4-fluorophenyl)-5- fluoropyrazin-2-yl)-4- methylisoquinolin- 1(2H)-one | 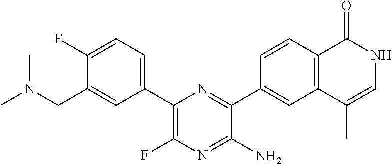 [SMILES: Cc1c[nH]c(=O)c2ccc(-c3nc(-c4ccc(F)c(CN(C)C)c4)c(F)nc3N)cc12] | B | |
| I-276 | 6-(3-amino-6-(3- ((dimethylamino)methyl)- 4-fluorophenyl)-5- fluoropyrazin-2-yl)-4- fluoroisoquinolin-1(2H)- one | 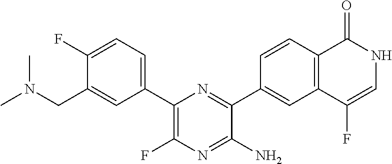 [SMILES: CN(C)Cc1cc(-c2nc(-c3ccc4c(=O)[nH]cc(F)c4c3)c(N)nc2F)ccc1F] | B | |
| I-277 | 6-(3-amino-6-(3- ((dimethylamino)methyl) phenyl)-5- fluoropyrazin-2-yl)-3,4- dihydroisoquinolin- 1(2H)-one | 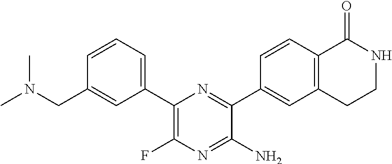 [SMILES: CN(C)Cc1cccc(-c2nc(-c3ccc4c(c3)CCNC4=O)c(N)nc2F)c1] | B | |
| I-278 | 6-(3-amino-6-(3- ((dimethylamino)methyl) phenyl)-5- fluoropyrazin-2-yl)-7- fluoro-3,4- dihydroisoquinolin- 1(2H)-one | 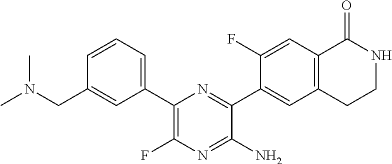 [SMILES: CN(C)Cc1cccc(-c2nc(-c3cc4c(cc3F)C(=O)NCC4)c(N)nc2F)c1] | B | |
| I-279 | 6-(3-amino-6-(3- ((dimethylamino)methyl) phenyl)-5- fluoropyrazin-2-yl)-8- fluoro-3,4- dihydroisoquinolin- 1(2H)-one | 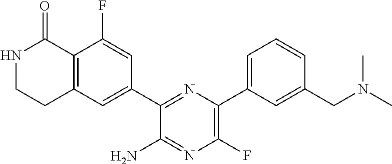 [SMILES: CN(C)Cc1cccc(-c2nc(-c3cc(F)c4c(c3)CCNC4=O)c(N)nc2F)c1] | B | |
| I-280 | 6-(3-amino-6-(3- ((dimethylamino)methyl) phenyl)-5- fluoropyrazin-2-yl)-4- methylisoquinolin- 1(2H)-one | 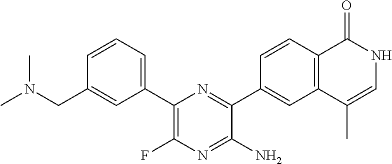 [SMILES: Cc1c[nH]c(=O)c2ccc(-c3nc(-c4cccc(CN(C)C)c4)c(F)nc3N)cc12] | B | |
| I-281 | 6-(3-amino-6-(3- ((dimethylamino)methyl) phenyl)-5- fluoropyrazin-2-yl)-4- fluoroisoquinolin-1(2H)- one | 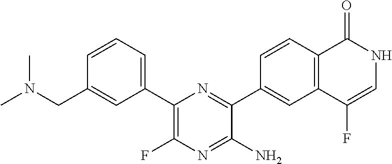 [SMILES: CN(C)Cc1cccc(-c2nc(-c3ccc4c(=O)[nH]cc(F)c4c3)c(N)nc2F)c1] | B | |
| I-282 | 6-(3-amino-6-(4-(3,6- dihydro-2H-pyran-4-yl)- 3- ((ethyl(methyl)amino) methyl)phenyl)-5- fluoropyrazin-2-yl)-4- fluoroisoquinolin-1(2H)- one | 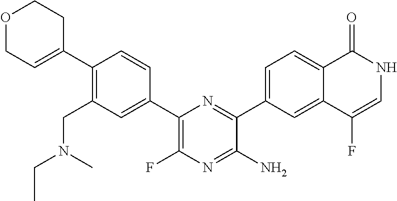 [SMILES: CCN(C)Cc1cc(-c2nc(-c3ccc4c(=O)[nH]cc(F)c4c3)c(N)nc2F)ccc1C1=CCOCC1] | B | |
| I-283 | (S)-6-(3-amino-6-(3- (dimethylamino)-2,3- dihydrobenzofuran-5- yl)-5-fluoropyrazin-2- yl)isoquinolin-1(2H)- one | 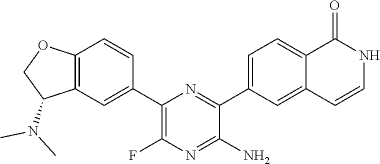 [SMILES: CN(C)[C@@H]1COc2ccc(-c3nc(-c4ccc5c(=O)[nH]ccc5c4)c(N)nc3F)cc21] | B | |
| I-284 | (S)-6-(3-amino-6-(3- (dimethylamino)-2,3- dihydrobenzofuran-5- yl)-5-fluoropyrazin-2- yl)-4-methylisoquinolin- 1(2H)-one | 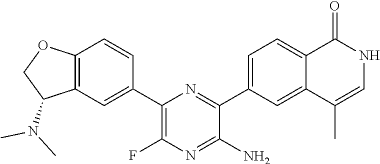 [SMILES: Cc1c[nH]c(=O)c2ccc(-c3nc(-c4ccc5c(c4)[C@H](N(C)C)CO5)c(F)nc3N)cc12] | B | |
| I-285 | (R)-6-(3-amino-6-(4- (dimethylamino)chroman- 6-yl)-5-fluoropyrazin- 2-yl)-4- methylisoquinolin- 1(2H)-one | 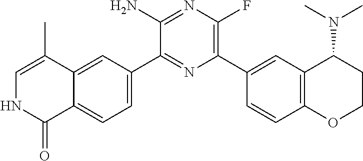 [SMILES: Cc1c[nH]c(=O)c2ccc(-c3nc(-c4ccc5c(c4)[C@H](N(C)C)CCO5)c(F)nc3N)cc12] | A | |
| I-286 | (R)-6-(3-amino-6-(4- (dimethylamino)chroman- 6-yl)-5-fluoropyrazin- 2-yl)isoquinolin-1(2H)- one | 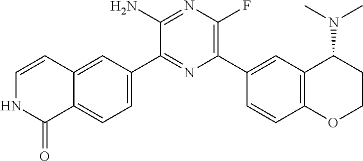 [SMILES: CN(C)[C@@H]1CCOc2ccc(-c3nc(-c4ccc5c(=O)[nH]ccc5c4)c(N)nc3F)cc21] | B | |
| I-287 | (R)-6-(3-amino-6-(4- (dimethylamino)chroman- 6-yl)-5-fluoropyrazin- 2-yl)-3,4- dihydroisoquinolin- 1(2H)-one | 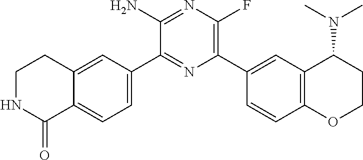 [SMILES: CN(C)[C@@H]1CCOc2ccc(-c3nc(-c4ccc5c(c4)CCNC5=O)c(N)nc3F)cc21] | A | |
| I-288 | (R)-6-(3-amino-6-(4- (dimethylamino)chroman- 6-yl)-5-fluoropyrazin- 2-yl)-7-fluoro-3,4- dihydroisoquinolin- 1(2H)-one | 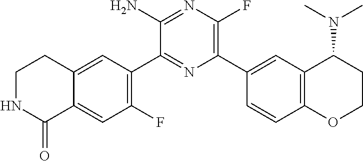 [SMILES: CN(C)[C@@H]1CCOc2ccc(-c3nc(-c4cc5c(cc4F)C(=O)NCC5)c(N)nc3F)cc21] | B | |
| I-289 | (R)-6-(3-amino-6-(4- (dimethylamino)chroman- 6-yl)-5-fluoropyrazin- 2-yl)-8-fluoro-3,4- dihydroisoquinolin- 1(2H)-one | 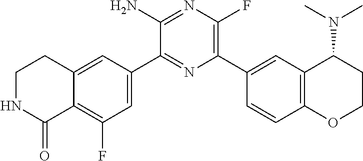 [SMILES: CN(C)[C@@H]1CCOc2ccc(-c3nc(-c4cc(F)c5c(c4)CCNC5=O)c(N)nc3F)cc21] | B | |
| I-290 | 6-(3-amino-6-(3-(1- (dimethylamino)ethyl)- 4-methoxyphenyl)-5- fluoropyrazin-2-yl)-3,4- dihydroisoquinolin- 1(2H)-one | 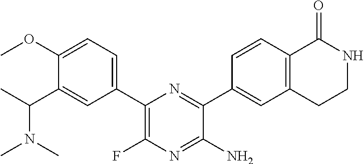 [SMILES: COc1ccc(-c2nc(-c3ccc4c(c3)CCNC4=O)c(N)nc2F)cc1C(C)N(C)C] | A | |
| I-291 | 6-(3-amino-6-(3-(1- (dimethylamino)ethyl)- 4-methoxyphenyl)-5- fluoropyrazin-2-yl)-4- fluoroisoquinolin-1(2H)- one | 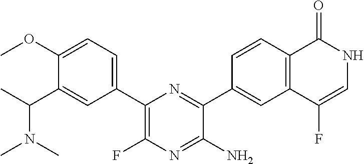 [SMILES: COc1ccc(-c2nc(-c3ccc4c(=O)[nH]cc(F)c4c3)c(N)nc2F)cc1C(C)N(C)C] | A | |
| I-292 | 5-(5-amino-3-fluoro-6- (1-oxo-1,2,3,4- tetrahydroisoquinolin-6- yl)pyrazin-2-yl)-2- methoxybenzonitrile | 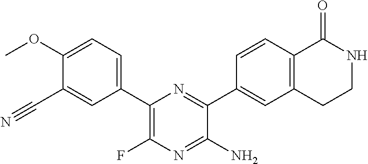 [SMILES: COc1ccc(-c2nc(-c3ccc4c(c3)CCNC4=O)c(N)nc2F)cc1C#N] | B | |
| I-293 | 6-(3-amino-6-(3- ((dimethylamino) methyl)-4- (isopropylsulfonyl) phenyl)-5- fluoropyrazin-2- yl)-4-fluoroisoquinolin- 1(2H)-one | 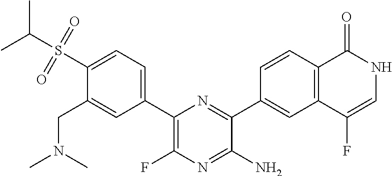 [SMILES: CC(C)S(=O)(=O)c1ccc(-c2nc(-c3ccc4c(=O)[nH]cc(F)c4c3)c(N)nc2F)cc1CN(C)C] | B | D |
| I-294 | 6-(3-amino-6-(2,5- dimethyl-1,2,3,4- tetrahydroisoquinolin-7- yl)-5-fluoropyrazin-2- yl)-3,4- dihydroisoquinolin- 1(2H)-one | 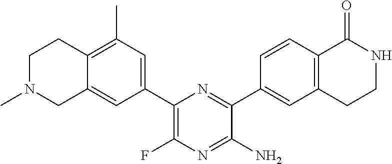 [SMILES: Cc1cc(-c2nc(-c3ccc4c(c3)CCNC4=O)c(N)nc2F)cc2c1CCN(C)C2] | A | |
| I-295 | 6-(3-amino-6-(2,5- dimethyl-1,2,3,4- tetrahydroisoquinolin-7- yl)-5-fluoropyrazin-2- yl)-4-methylisoquinolin- 1(2H)-one | 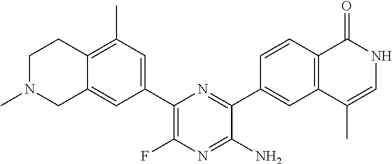 [SMILES: Cc1cc(-c2nc(-c3ccc4c(=O)[nH]cc(C)c4c3)c(N)nc2F)cc2c1CCN(C)C2] | B | |
| I-296 | 6-(3-amino-6-(2,5- dimethyl-1,2,3,4- tetrahydroisoquinolin-7- yl)-5-fluoropyrazin-2- yl)-4-fluoroisoquinolin- 1(2H)-one | 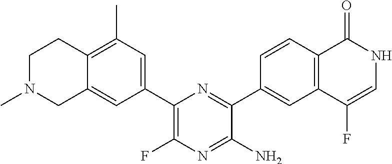 [SMILES: Cc1cc(-c2nc(-c3ccc4c(=O)[nH]cc(F)c4c3)c(N)nc2F)cc2c1CCN(C)C2] | A | |
| I-297 | 6-(3-amino-6-(3- ((ethyl(methyl)amino) methyl)-4-methoxy-5- methylphenyl)-5- fluoropyrazin-2-yl)-3,4- dihydroisoquinolin- 1(2H)-one | 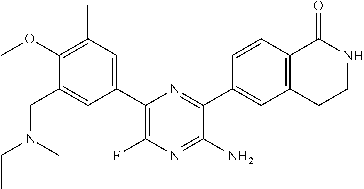 [SMILES: CCN(C)Cc1cc(-c2nc(-c3ccc4c(c3)CCNC4=O)c(N)nc2F)cc(C)c1OC] | B | |
| I-298 | 6-(3-amino-6-(3- ((ethyl(methyl)amino) methyl)-4-methoxy-5- methylphenyl)-5- fluoropyrazin-2-yl)-4- fluoroisoquinolin-1(2H)- one | 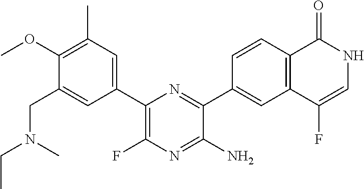 [SMILES: CCN(C)Cc1cc(-c2nc(-c3ccc4c(=O)[nH]cc(F)c4c3)c(N)nc2F)cc(C)c1OC] | B | |
| I-299 | 6-(3-amino-6-(3- ((ethyl(methyl)amino) methyl)-4-methoxy-5- methylphenyl)-5- fluoropyrazin-2-yl)-4- methylisoquinolin- 1(2H)-one | 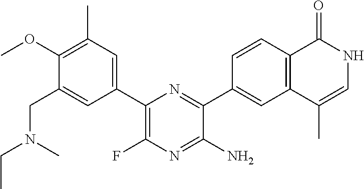 [SMILES: CCN(C)Cc1cc(-c2nc(-c3ccc4c(=O)[nH]cc(C)c4c3)c(N)nc2F)cc(C)c1OC] | B | |
| I-300 | 6-(3-amino-6-(3- ((ethyl(methyl)amino) methyl)-4- methoxyphenyl)-5- fluoropyrazin-2-yl)-4- fluoroisoquinolin-1(2H)- one | 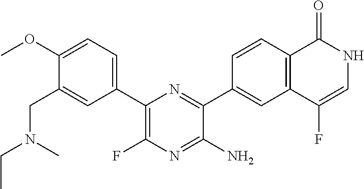 [SMILES: CCN(C)Cc1cc(-c2nc(-c3ccc4c(=O)[nH]cc(F)c4c3)c(N)nc2F)ccc1OC] | B | |
| I-301 | 6-(3-amino-6-(3- ((ethyl(methyl)amino) methyl)-4- methoxyphenyl)-5- fluoropyrazin-2-yl)-4- methylisoquinolin- 1(2H)-one | 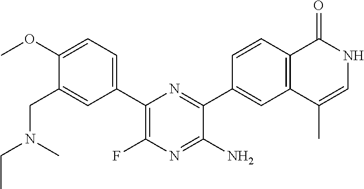 [SMILES: CCN(C)Cc1cc(-c2nc(-c3ccc4c(=O)[nH]cc(C)c4c3)c(N)nc2F)ccc1OC] | B | |
| I-302 | 6-(3-amino-6-(3-(1- (dimethylamino)ethyl)- 4-hydroxyphenyl)-5- fluoropyrazin-2-yl)-4- fluoroisoquinolin-1(2H)- one | 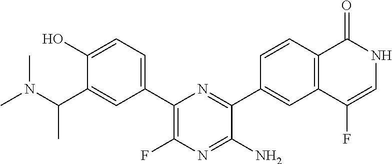 [SMILES: CC(c1cc(-c2nc(-c3ccc4c(=O)[nH]cc(F)c4c3)c(N)nc2F)ccc1O)N(C)C] | A | |
| I-303 | 6-(3-amino-6-(3- ((ethyl(methyl)amino) methyl)-4-methoxy-5- methylphenyl)-5- fluoropyrazin-2-yl)-7- fluoro-3,4- dihydroisoquinolin- 1(2H)-one | 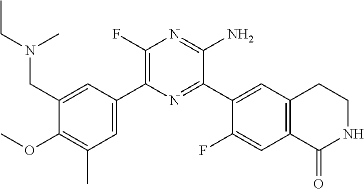 [SMILES: CCN(C)Cc1cc(-c2nc(-c3cc4c(cc3F)C(=O)NCC4)c(N)nc2F)cc(C)c1OC] | A | |
| I-304 | 6-(3-amino-6-(3-(1- (dimethylamino)ethyl)- 4-hydroxyphenyl)-5- fluoropyrazin-2-yl)-4- methylisoquinolin- 1(2H)-one | 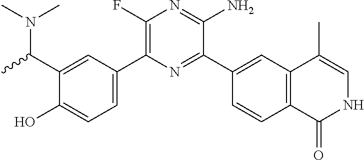 [SMILES: Cc1c[nH]c(=O)c2ccc(-c3nc(-c4ccc(O)c(C(C)N(C)C)c4)c(F)nc3N)cc12] | A | |
| I-305 | 6-(3-amino-6-(3- ((dimethylamino)methyl)- 4-hydroxyphenyl)-5- fluoropyrazin-2-yl)-4- fluoroisoquinolin-1(2H)- one | 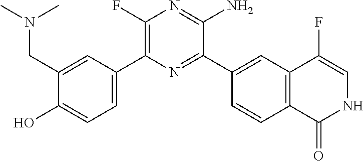 [SMILES: CN(C)Cc1cc(-c2nc(-c3ccc4c(=O)[nH]cc(F)c4c3)c(N)nc2F)ccc1O] | A | |
| I-306 | 6-(3-amino-6-(3- ((dimethylamino)methyl)- 4-hydroxyphenyl)-5- fluoropyrazin-2-yl)-4- methylisoquinolin- 1(2H)-one | 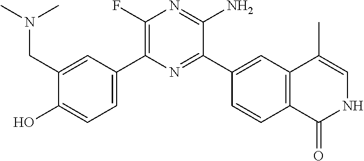 [SMILES: Cc1c[nH]c(=O)c2ccc(-c3nc(-c4ccc(O)c(CN(C)C)c4)c(F)nc3N)cc12] | B | |

*A: 0.1-10 nM; B: 11-100 nM; C: 101-1000 nM; D > 1000 nM

TABLE 3
Comparative Compounds

| Comparative | | |
|---|---|---|
| Compound I.D. | IUPAC chemical name | Structure |
| Comparator 1 | 6-(3-amino-6-(4- morpholinophenyl)pyrazin-2-yl)-3,4- dihydroisoquinolin-1(2H)-one | 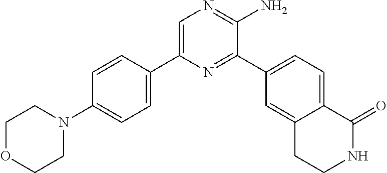 [SMILES: Nc1ncc(-c2ccc(N3CCOCC3)cc2)nc1-c1ccc2c(c1)CCNC2=O] |

TABLE 4
LcK IC₅₀/HPK IC₅₀ selectivity

| Structure | Selectivity |
|---|---|
| 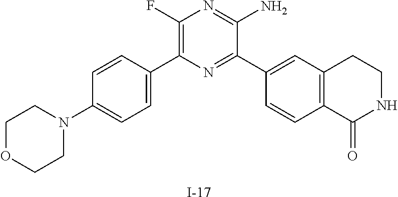 | >1666 |
| 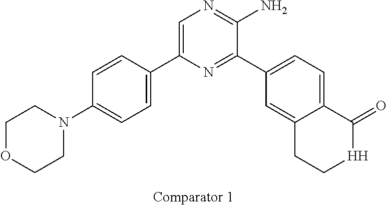 | 31 |

1. A compound of Formula (I) or an enantiomer thereof, or a pharmaceutically acceptable salt, solvate and/or prodrug thereof, 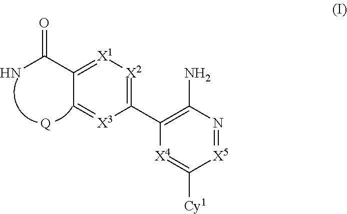
wherein:
X¹ is selected from N and CR¹;
X² and X³ are independently selected from N and CR²;
one of X⁴ and X⁵ is N and the other is CR³;
Q is C₁₋₄alkylene optionally interrupted by a heteroatom selected from O, S, S(O), SO₂, and NR⁴ and/or optionally substituted with one or more of R⁵ and/or optionally disubstituted on one carbon with R^{5a} and R^{5b}; provided that when Q comprises the heteromoiety the heteromoiety is not separated from the ring amide NH by methylene; or
Q is C₂₋₄alkenylene optionally substituted with one or more of R^{5c}; or
Q is optionally selected from C═N and N═C, and is optionally substituted with R^{5c};
R¹ is selected from H, halo, OR⁶, NR⁷R⁸, C₁₋₆alkyleneNR⁷R⁸ and C₁₋₆alkyl;
R² is selected from H, halo and C₁₋₆alkyl;
R³ is halo;
R⁴ is selected from H and C₁₋₆alkyl;
each R⁵ is independently selected from ═O, halo, C₁₋₆alkyl, C₃₋₆cycloalkyl, C₃₋₆heterocycloalkyl, C₁₋₆alkyleneC₃₋₆cycloalkyl, C₁₋₆alkyleneC₃₋₆heterocycloalkyl, OH, OC₁₋₆alkyl, NR⁹R¹⁰ and C₁₋₆alkyleneNR⁹R¹⁰;
R^{5a} and R^{5b} are joined to form, together with the carbon atom therebetween, a 3- to 6-membered, saturated or unsaturated ring optionally containing one heteromoiety selected from N, NH, NC₁₋₆alkyl, O, S, S(O), and SO₂ and optionally substituted with one or more of halo and C₁₋₆alkyl;
each R^{5c} is independently selected from halo, C₁₋₆alkyl, C₃₋₆cycloalkyl, C₃₋₆heterocycloalkyl, C₁₋₆alkyleneC₃₋₆cycloalkyl, C₁₋₆alkyleneC₃₋₆heterocycloalkyl, OH, OC₁₋₆alkyl NR⁹R¹⁰, and C₁₋₆alkyleneNR⁹R¹⁰;
R⁶ is selected from H and C₁₋₆alkyl;
R⁷, R⁸, R⁹ and R¹⁰ are each independently selected from H and C₁₋₆alkyl; or
R⁷ and R⁸ or R⁹ and R¹⁰ are joined to form, together with the nitrogen atom therebetween, a 3- to 7-membered saturated or unsaturated ring, optionally containing one additional heteromoiety selected from N, NH, NC₁₋₆alkyl, O, S, SO, and SO₂ and optionally substituted with one or more of halo and C₁₋₆alkyl;
Cy¹ is C₆₋₂₀aryl or C₅₋₂₀heteroaryl, and Cy¹ is unsubstituted or is substituted with one or more of R¹¹, or
Cy¹ is substituted with Z-Cy², or
Cy¹ is substituted with Z-Cy² and one or more of R¹¹;
each R¹¹ is independently selected from halo, ═O, CN, NO₂, C₁₋₆alkyl, C₂₋₆alkenyl, C₂₋₆alkynyl, OR¹², C(O)R¹², CO₂R¹², P(O)R¹²R¹³, P(O)(OR¹²)(OR¹³), SR¹², S(O)R¹², SO₂R¹², S(O)(═NR¹³)R¹², SO₂NR¹²R¹³, SiR¹⁴R^{14a}R^{14b}, C₁₋₆alkyleneOR¹², OC₁₋₆alkyleneOR¹², C₁₋₆alkyleneNR^{12a}R^{13a}, OC₁₋₆alkyleneNR¹²R¹³, NR^{13a}C₁₋₆alkyleneNR¹²R¹³, NR^{13a}C₁₋₆alkyleneOR¹², NR^{12a}R^{13a}, NR^{13a}COR¹², NR^{13a}CO₂R¹², NR^{13a}SO₂R¹², C₃₋₇cycloalkyl, C₃₋₇heterocycloalkyl, C₁₋₆alkyleneC₃₋₇cycloalkyl and C₁₋₆alkyleneC₃₋₇heterocycloalkyl, the latter four groups being optionally substituted with one or more of R¹⁵;
R¹² is selected from H, C₁₋₆alkyl, C₂₋₆alkenyl, C₂₋₆alkynyl, C₁₋₆alkyleneC₃₋₁₀cycloalkyl, C₁₋₆alkyleneC₃₋₁₀heterocycloalkyl, C₁₋₆alkyleneOR¹⁶, and C₁₋₆alkyleneNR^{16a}R^{16b}, and alkyl, alkenyl, alkynyl, alkylene, heterocycloalkyl and cycloalkyl groups of R¹² are optionally substituted with one or more of R¹⁷;
R¹³ is selected from H and C₁₋₆alkyl; or
R¹² and R¹³ are joined to form, together with the atom(s) therebetween, a 4- to 6-membered saturated or unsaturated ring, optionally containing one additional heteromoiety selected from N, NR¹⁸, O, S, SO, and SO₂, and optionally substituted with one or more of R¹⁷;
R^{12a} is selected from H, C₁₋₆alkyl, C₂₋₆alkenyl, C₂₋₆alkynyl, C₁₋₆alkyleneC₃₋₁₀cycloalkyl, C₁₋₆alkyleneC₃₋₁₀heterocycloalkyl, C₁₋₆alkyleneOR¹⁶, and C₁₋₆alkyleneNR^{16a}R^{16b}, and all alkyl, alkenyl, alkynyl, alkylene, heterocycloalkyl and cycloalkyl groups of R^{12a} are optionally substituted with one or more of R¹⁷;
R^{13a} is selected from H and C₁₋₆alkyl;
R¹⁴, R^{14a}, and R^{14b} are independently selected from OR¹⁹, C₁₋₆alkyl, C₃₋₁₁cycloalkyl, C₃₋₁₀heterocycloalkyl, C₁₋₆alkyleneC₃₋₁₀cycloalkyl, and C₁₋₆alkyleneC₃₋₁₀heterocycloalkyl;
each R¹⁵ is independently selected from halo, OH, C₁₋₆alkyl, OC₁₋₆alkyl, CN and NR^{15a}R^{15b};
R^{15a} and R^{15b} are each independently selected from H and C₁₋₆alkyl;
R¹⁶, R^{16a} and R^{16b} are each independently selected from H and C₁₋₆alkyl;
each R¹⁷ is independently selected from halo, C₁₋₆alkyl, CN and NR^{17a}R^{17b};
R^{17a} and R^{17b} are each independently selected from H and C₁₋₆alkyl;
R¹⁸ is selected from H and C₁₋₆alkyl;
R¹⁹ is selected from H, C₁₋₆alkyl, C₃₋₁₁cycloalkyl, C₃₋₁₀heterocycloalkyl, C₁₋₆alkyleneC₃₋₁₀cycloalkyl, C₁₋₆alkyleneC₃₋₁₀heterocycloalkyl C₁₋₆alkyleneOR²⁰, and C₁₋₆alkyleneNR²⁰R²¹;
R²⁰ and R²¹ are independently selected from H and C₁₋₆alkyl;
Z is absent, or is selected from C₁₋₆alkylene, O, C(O), CO₂, S, S(O), SO₂, S(O)(═NR^{13b}), NR^{13b}, C₁₋₆alkyleneO, C₁₋₆alkyleneC(O), C₁₋₆alkyleneCO₂, C₁₋₆alkyleneS, C₁₋₆alkyleneS(O), C₁₋₆alkyleneSO₂, C₁₋₆alkyleneS(O)(═NR^{13b}), C₁₋₆alkyleneNR^{13b}, OC₁₋₆alkylene, C(O)C₁₋₆alkylene, CO₂C₁₋₆alkylene, SC₁₋₆alkylene, S(O)C₁₋₆alkylene, SO₂C₁₋₆alkylene, S(O)(═NR^{13b})C₁₋₆alkylene and NR^{13b}C₁₋₆alkylene;
R^{13b} is selected from H and C₁₋₆alkyl;
Cy² is selected from C₃₋₁₄cycloalkyl and C₃₋₁₄heterocycloalkyl, and Cy² is unsubstituted or substituted with one or more of R²²;
each R²² is independently selected from halo, ═O, CN, OH, C₁₋₆alkyl, C₂₋₆alkenyl, C₂₋₆alkynyl, C₃₋₁₀cycloalkyl, C₃₋₁₀heterocycloalkyl, C₆₋₁₁aryl, C₅₋₁₄heteroaryl, C₁₋₆alkyleneC₃₋₁₀cycloalkyl, C₁₋₆alkyleneC₃₋₁₀heterocycloalkyl, C₁₋₆alkyleneC₆₋₁₁aryl, C₁₋₆alkyleneC₅₋₁₄heteroaryl, OC₁₋₆alkyl, OC₂₋₆alkenyl, OC₂₋₆alkynyl, C₁₋₆alkyleneOR²³, OC₁₋₆alkyleneOR²³, C₁₋₆alkyleneNR²⁴R²⁵, OC₁₋₆alkyleneNR²⁴R²⁵, SC₁₋₆alkyl, SC₂₋₆alkenyl, SC₂₋₆alkynyl, C(O)C₁₋₆alkyl, C(O)C₂₋₆alkenyl, C(O)C₂₋₆alkynyl, C(O)C₃₋₁₀cycloalkyl, C(O)C₃₋₁₀heterocycloalkyl, C(O)C₆₋₁₁aryl, C(O)C₅₋₁₄heteroaryl, C(O)C₁₋₆alkyleneC₃₋₁₀cycloalkyl, C(O)C₁₋₆alkyleneC₃₋₁₀heterocycloalkyl, C(O)C₁₋₆alkyleneC₆₋₁₁aryl, C(O)C₁₋₆alkyleneC₅₋₁₄heteroaryl, C(O)C₁₋₆alkylenylOR²³, C(O)C₁₋₆alkyleneNR²⁴R²⁵, C(O)C₁₋₆alkyleneOC₁₋₆alkyleneNR²⁴R²⁵, C(O)NR²⁴R²⁵, CO₂C₁₋₆alkyl, CO₂C₂₋₆alkenyl, CO₂C₂₋₆alkynyl, CO₂C₁₋₆alkyleneOR²³, CO₂C₁₋₆alkyleneOC₁₋₆alkyleneNR²⁴R²⁵, NR²⁴R²⁵, NR²⁶C₃₋₁₀cycloalkyl, NR²⁶C₃₋₁₀heterocycloalkyl, NR²⁶C₆₋₁₁aryl, NR²⁶C₅₋₁₄heteroaryl, NR²⁶C₁₋₆alkyleneOR²³, NR²⁶C₁₋₆alkyleneNR²⁴R²⁵, NR²⁶C₁₋₆alkyleneC₃₋₁₀cycloalkyl, NR²⁶C₁₋₆alkyleneC₃₋₁₀heterocycloalkyl, NR²⁶C₁₋₆alkyleneC₆₋₁₁aryl, NR²⁶C₁₋₆alkyleneC₅₋₁₄heteroaryl, NR²⁶SO₂C₁₋₆alkyl, SO₂C₁₋₆alkyl, SO₂C₂₋₆alkenyl, SO₂C₂₋₆alkynyl, and SO₂NR²⁴R²¹, and alkyl, alkenyl, alkynyl, alkylene, aryl, heteroaryl, heterocycloalkyl and cycloalkyl groups of R²² are optionally substituted with one or more of R²⁷;
R^{22b} and R^{22c} are each independently selected from H and C₁₋₆alkyl;
R²³ is selected from H, C₁₋₆alkyl, C₁₋₆alkyleneOC₁₋₆alkyl, C₃₋₁₁cycloalkyl, C₃₋₁₀heterocycloalkyl, C₆₋₁₁aryl, C₅₋₁₄heteroaryl, C₂₋₆alkenyl, C₂₋₆alkynyl, C₁₋₆alkyleneC₃₋₁₀cycloalkyl, C₁₋₆alkyleneC₃₋₁₀heterocycloalkyl, C₁₋₆alkyleneC₆₋₁₁aryl, and C₁₋₆alkyleneC₅₋₁₄heteroaryl;
R²⁴ is selected from H and C₁₋₆alkyl;
R²⁵ is selected from H, C₁₋₆alkyl, C₁₋₆alkyleneOC₁₋₆alkyl, C₃₋₁₀cycloalkyl, C₃₋₁₀heterocycloalkyl, C₆₋₁₁aryl, C₅₋₁₄heteroaryl, C₁₋₆alkyleneC₃₋₁₀cycloalkyl, C₁₋₆alkyleneC₃₋₁₀heterocycloalkyl, C₁₋₆alkyleneC₆₋₁₁aryl, and C₁₋₆alkyleneC₅₋₁₄heteroaryl, or
R²⁴ and R²⁵ are joined to form, together with the nitrogen atom therebetween, a 4- to 6-membered saturated or unsaturated ring, optionally containing one additional heteromoiety selected from N, NR²⁸, O, S, S(O), and SO₂, and optionally substituted with one or more of halo and C₁₋₆alkyl;
R²⁶ is selected from H and C₁₋₆alkyl;
each R²⁷ is independently selected from halo, C₁₋₆alkyl, CN and NR^{27a}R^{27b}; and
R^{27a}, R^{27b} and R²⁸ are each independently selected from H and C₁₋₆alkyl;
wherein all available hydrogen atoms are optionally substituted with a fluorine atom.

2. The compound of claim 1, wherein X¹ is N or wherein X¹ is CR¹ and R¹ is selected from H, F, Cl and C₁₋₄alkyl, OR⁶, NR⁷R⁸, C₁₋₄alkyleneNR⁷R⁸ and C₁₋₄alkyl, wherein all available hydrogen atoms are optionally substituted with a fluorine atom.

3. (canceled)

4. (canceled)

5. The compound of claim 1, wherein Q is C₁₋₃alkylene optionally substituted with one to three of R⁵, each R⁵ is independently selected from ═O, F, Cl, C₁₋₄alkyl, C₃₋₆cycloalkyl, C₃₋₆heterocycloalkyl, C₁₋₄alkyleneC₃₋₆cycloalkyl, C₁₋₄alkyleneC₃₋₆heterocycloalkyl, OH, OC₁₋₄alkyl, NR⁹R¹⁰ and C₁₋₄alkyleneNR⁹R¹⁰, and R⁹ and R¹⁰ are each independently selected from H and C₁₋₄alkyl, or
Q is C₁₋₃alkylene optionally interrupted by NR⁴, wherein R⁴ is selected from H and C₁₋₄alkyl; or
Q is C₁₋₃alkylene and optionally disubstituted on one carbon atom with R^{5a} and R^{5b} and R^{5a} and R^{5b} are joined to form, together with the nitrogen atom therebetween, a 3- to 6-membered saturated or unsaturated ring optionally containing one additional heteromoiety selected from N, NH, NC₁₋₆alkyl, O, S, S(O), and SO₂ and optionally substituted with one or more of halo and C₁₋₄alkyl, or
Q is C₂₋₄alkenylene optionally substituted with one or two of R^{5c}, and each R^{5c} is independently selected from F, C₁₋₄alkyl, C₃₋₆cycloalkyl, C₃₋₆heterocycloalkyl, C₁₋₄alkyleneC₃₋₆cycloalkyl, C₁₋₄alkyleneC₃₋₆heterocycloalkyl, OH, OC₁₋₆alkyl, NR⁹R¹⁰, and C₁₋₄alkyleneNR⁹R¹⁰, or
Q is selected from C═N and N═C and is optionally substituted with R^{5c}, each R^{5c} is independently selected from F, C₁₋₄alkyl, C₃₋₆cycloalkyl, C₃₋₆heterocycloalkyl, C₁₋₄alkyleneC₃₋₆cycloalkyl, C₁₋₄alkyleneC₃₋₆heterocycloalkyl, OH, OC₁₋₆alkyl, NR⁹R¹⁰ and C₁₋₄alkyleneNR⁹R¹⁰, and R⁹ and R¹⁰ are each independently selected from H and C₁₋₄alkyl, and
wherein all available hydrogen atoms are optionally substituted with a fluorine atom.

6.-18. (canceled)

19. The compound of claim 1, wherein one of X² and X³ is N and the other is CR², or
both X² and X³ are CR² and each R² is independently selected from H and F.

20.-24. (canceled)

25. The compound of claim 1, wherein X⁴ is N and X⁵ is CR³.

26. The compound of claim 1, wherein X⁴ is N and X⁵ is CR³ and R³ is F.

27. The compound of claim 1, wherein Cy¹ is phenyl, and Cy¹ is unsubstituted, or is substituted with one or more of R⁹, or is substituted with Z-Cy², or is substituted with Z-Cy² and one or more of R¹¹, or
Cy¹ is a bicyclic C₉₋₁₁aryl wherein the aryl is fused to a heterocycloalkyl, and Cy¹ is unsubstituted, or is substituted with one or more of R¹¹, or is substituted with Z-Cy², or is substituted with Z-Cy² and one or more of R¹¹, or
Cy¹ is a tricyclic C₁₁₋₁₇aryl wherein the aryl group is fused and/or spiro fused to one or two heterocycloalkyl groups, and Cy¹ is unsubstituted, or is substituted with one or more of R¹¹ or is substituted with Z-Cy², or is substituted with Z-Cy² and one or more of R¹¹.

28.-34. (canceled)

35. The compound of claim 1, wherein Cy¹ is C₅₋₆heteroaryl, and Cy¹ is unsubstituted, or is substituted with one or more of R¹¹, or is substituted with Z-Cy², or is substituted with Z-Cy² and one or more of R¹¹, or
Cy¹ is bicyclic C₉₋₁₁heteroaryl, and Cy¹ is unsubstituted, or is substituted with one or more of R¹¹, or is substituted with Z-Cy², or is substituted with Z-Cy² and one or more of R¹¹.

36.-40. (canceled)

41. The compound of claim 1, wherein each R¹¹ is independently selected from F, Cl, ═O, CN, NO₂, C₁₋₄alkyl, OR¹², C(O)R¹², CO₂R¹², P(O)R¹²R¹³, P(O)(OR¹²)(OR¹³), SR¹², SOR¹², SO₂R¹², S(O)(═NR¹³)R¹², SO₂NR¹²R¹³, SiR¹⁴R^{14a}R^{14b}, C₁₋₆₄alkyleneOR¹², OC₁₋₆alkyleneOR¹², C₁₋₆alkyleneNR^{12a}R^{13a}, OC₁₋₄alkyleneNR¹²R¹³, NR^{13a}C₁₋₄alkyleneNR¹²R¹³, NR^{13a}C₁₋₄alkyleneOR¹², NR^{12a}R^{13a}, NR^{13a}COR¹², NR^{13a}CO₂R¹², and NR^{13a}SO₂R¹², and NR^{13a}SO₂R¹², C₃₋₇cycloalkyl, C₃₋₇heterocycloalkyl, C₁₋₄alkyleneC₃₋₇cycloalkyl and C₁₋₄alkyleneC₃₋₆heterocycloalkyl, the latter four groups being optionally substituted with one to three of R¹⁵,
each R¹⁵ is independently selected from F, Cl, CN, OH, C₁₋₄alkyl, OC₁₋₄alkyl and NR^{15a}R^{15b}, and R^{15a} and R^{15b} are each independently selected from H and C₁₋₄alkyl,
wherein all available hydrogen atoms in the C₁₋₄alkyl group are optionally substituted with a fluorine atom.

42.-68. (canceled)

69. The compound of claim 1, wherein each R¹⁷ is independently selected from F, Cl, CN, C₁₋₄alkyl and NR^{17a}R^{17b}, and
R^{17a} and R^{17b} are independently selected from H and C₁₋₄alkyl,
wherein all available hydrogen atoms in the C₁₋₄alkyl group are optionally substituted with a fluorine atom.

70. (canceled)

71. The compound of claim 41, wherein one of R¹⁴, R^{14a} and R^{14b} is OR¹⁹ and the other two of R¹⁴, R^{14a} and R^{14b} are independently selected from C₁₋₄alkyl, C₃₋₁₁cycloalkyl, C₃₋₁₀heterocycloalkyl, C₁₋₄alkyleneC₃₋₁₀cycloalkyl, and C₁₋₄alkyleneC₃₋₁₀heterocycloalkyl wherein all available hydrogen atoms optionally substituted with a fluorine atom,
R¹⁹ is selected from H, C₁₋₄alkyl, C₃₋₁₁cycloalkyl, C₃₋₁₀heterocycloalkyl, C₁₋₄alkyleneC₃₋₁₀cycloalkyl, C₁₋₄alkyleneC₃₋₁₀heterocycloalkyl C₁₋₄alkyleneOR²⁰, and C₁₋₄alkyleneNR²⁰R²¹, and
R²⁰ and R²¹ are independently selected from H and C₁₋₄alkyl,
wherein all available hydrogen atoms in the C₁₋₄alkyl group are optionally substituted with a fluorine atom.

72.-97. (canceled)

98. The compound of claim 1, wherein Z is absent, or Z is selected from C₁₋₄alkylene, O, C(O), and SO₂ wherein all available hydrogen atoms are optionally substituted with a fluorine atom.

99.-102. (canceled)

103. The compound of claim 1, wherein Z is selected from C₁₋₄alkyleneO, C₁₋₄alkyleneC(O), C₁₋₆alkyleneCO₂, C₁₋₄alkyleneS, C₁₋₄alkyleneS(O), C₁₋₄alkyleneSO₂, C₁₋₄alkyleneNR^{13b}, OC₁₋₄alkylene, C(O)C₁₋₄alkylene, CO₂C₁₋₄alkylene, SC₁₋₄alkylene, S(O)C₁₋₄alkylene, SO₂C₁₋₄alkylene, and NR^{13b}C₁₋₆alkylene wherein all available hydrogen atoms are optionally substituted with a fluorine atom.

104. (canceled)

105. (canceled)

106. The compound of claim 1, wherein Cy² is C₃₋₁₁cycloalkyl and Cy² is unsubstituted or substituted with one or more of R²², or
Cy² is a monocyclic C₃₋₇heterocycloalkyl and Cy² is unsubstituted or substituted with one or more of R²², or
Cy² is a bicyclic heterocycle and Cy² is unsubstituted or substituted with one or more of R²².

107.-121. (canceled)

122. The compound of claim 106, wherein each R²² is independently selected from F, Cl, CN, OH, NO₂, C₁₋₄alkyl, C₃₋₁₀cycloalkyl, C₃₋₁₀heterocycloalkyl, C₆₋₁₁aryl, C₅₋₁₄heteroaryl, C₁₋₂alkyleneC₃₋₁₀cycloalkyl, C₁₋₂alkyleneC₃₋₁₀heterocycloalkyl, C₁₋₄alkyleneC₆₋₁₁aryl, C₁₋₄alkyleneC₅₋₁₄heteroaryl, OC₁₋₄alkyl, C₁₋₄alkyleneOR²¹, C(O)C₁₋₄alkyl, C(O)C₃₋₁₀cycloalkyl, C(O)C₃₋₁₀heterocycloalkyl, C(O)C₁₋₄alkyleneC₃₋₁₀cycloalkyl, C(O)C₁₋₄alkyleneC₃₋₁₀heterocycloalkyl, C(O)C₁₋₄alkyleneOR²³, C(O)C₁₋₄alkyleneNR²⁴R²⁵, C(O)C₁₋₄alkyleneOC₁₋₄alkyleneNR²⁴R²⁵, CO₂C₁₋₆alkyl, CO₂C₁₋₄alkyleneOR²³, NR²⁴R²⁵, NR²⁶C₃₋₁₀cycloalkyl, NR²⁶C₃₋₁₀heterocycloalkyl, NR²⁶C₁₋₂alkyleneC₃₋₁₀cycloalkyl, NR²⁶C₁₋₂alkyleneC₃₋₁₀heterocycloalkyl, NR²⁶C₁₋₄alkyleneOR²³, NR²⁶SO₂C₁₋₄alkyl, and SO₂C₁₋₆alkyl, and alkyl, alkenyl, alkynyl, alkylene, aryl, heteroaryl, heterocycloalkyl and cycloalkyl groups of R²² are optionally substituted with one to three of R²⁷ wherein all available hydrogen atoms are optionally substituted with a fluorine atom, and
R²⁷ is selected from F, Cl, CN, C₁₋₄alkyl and NR^{27a}R^{27b}, and R^{27a} and R^{27b} are independently selected from H and C₁₋₄alkyl, wherein all available hydrogen atoms in the C₁₋₄alkyl group are optionally substituted with a fluorine atom, or
R²⁷ is selected from H, C₁₋₄alkyl, C₁₋₄alkyleneOC₁₋₆alkyl, C₃₋₁₁cycloalkyl, C₃₋₁₀heterocycloalkyl, C₂₋₄alkenyl, C₂₋₄alkynyl, C₁₋₄alkyleneC₃₋₁₀cycloalkyl, and C₁₋₄alkyleneC₃₋₁₀heterocycloalkyl wherein all available hydrogen atoms are optionally substituted with a fluorine atom wherein all available hydrogen atoms are optionally substituted with a fluorine atom.

123.-125. (canceled)

126. The compound of claim 122, wherein R²⁴ is selected from H and C₁₋₄alkyl and R²⁵ is selected from H, C₁₋₄alkyl, C₁₋₄alkyleneOC₁₋₄alkyl, C₃₋₁₀cycloalkyl, C₃₋₁₀heterocycloalkyl, C₁₋₄alkyleneC₃₋₁₀cycloalkyl, and C₁₋₄alkyleneC₃₋₁₀heterocycloalkyl wherein all available hydrogen atoms are optionally substituted with a fluorine atom, or
R²⁴ and R²⁵ are joined to form, together with the nitrogen atom therebetween, a 4- to 6-membered saturated or unsaturated ring, optionally containing one additional heteromoiety selected from NR²⁸, O and S, and optionally substituted with one to three of halo and C₁₋₆alkyl and C₁₋₆alkyl; and
R²⁸ is selected from H and C₁₋₄alkyl;
wherein all available hydrogen atoms in the C₁₋₄alkyl group are optionally substituted with a fluorine atom.

127.-131. (canceled)

132. The compound of claim 1, wherein the compound of Formula (I) is selected from the compounds listed in Table 1,
or a pharmaceutically acceptable salt, solvate and/or prodrug thereof.

133. (canceled)

134. (canceled)

135. (canceled)

136. A method of treating cancer comprising administering a therapeutically effective amount of one or more compounds of any one of claim 1 to a subject in need thereof.

137. (canceled)

138. A method of inhibiting proliferative activity in a cell, comprising administering an effective amount of one or more compounds of claim 1 to the cell.

139. (canceled)

140. (canceled)

141. A pharmaceutical composition comprising one or more compounds of claim 1, or a pharmaceutically acceptable salt, and/or solvate thereof, and a pharmaceutically acceptable carrier and/or diluent.

142. (canceled)
